(19) United States
(12) Patent Application Publication
(10) Pub. No.: US 2023/0146395 A1
(43) Pub. Date: May 11, 2023

(54) Substituted Pyrimidines and Uses Thereof

(71) Applicant: Verge Analytics, Inc., South San Francisco, CA (US)

(72) Inventors: Mark D. Rosen, San Diego, CA (US); Robert A. Galemmo, Jr., San Francisco, CA (US); Weiling Liang, Palo Alto, CA (US); Irene Y. Choi, Pacifica, CA (US); Brian Kopec, San Francisco, CA (US); Jane Rhodes, Belmont, MA (US)

(73) Assignee: Verge Analytics, Inc., South San Francisco, CA (US)

(21) Appl. No.: 17/910,082

(22) PCT Filed: Mar. 8, 2021
(86) PCT No.: PCT/US2021/021369

§ 371 Date: Sep. 8, 2022

Related U.S. Application Data
(60) Provisional application No. 62/987,289, filed on Mar. 9, 2020.
(60) Provisional application No. 63/093,059, filed on Oct. 16, 2020.

(51) Int. Cl. C07D 491/048

(52) U.S. Cl. CPC C07D 491/048; C07B 2200/05

(57) ABSTRACT

The present disclosure provides compounds that are inhibitors of PIKfyve and/or PI3 kinases, and are therefore useful for the treatment of neurological diseases treatable by inhibition of PIKfyve and/or PI3 kinases. Also provided are pharmaceutical compositions containing such compounds, and methods of treatment of neurological diseases using such compounds.

### CROSS-REFERENCE TO RELATED APPLICATIONS

This application claims priority to U.S. Provisional Application No. 62/987,289, filed on Mar. 9, 2020, and U.S. Provisional Application No. 63/093,059, filed on Oct. 16, 2020, the disclosures of each of which are hereby incorporated by reference in their entireties.

### FIELD OF INVENTION

The present disclosure provides compounds that are phosphoinositide kinase inhibitors, in particular FYVE-type finger-containing phosphoinositide kinase (“PIKfyve”) inhibitors and are therefore useful for the treatment of central nervous system diseases. Also provided are pharmaceutical compositions containing such compounds and processes for preparing such compounds.

### BACKGROUND

Phosphoinositide kinases (PIKs) catalyze the phosphorylation of phosphatidylinositol, which is a component of eukaryotic cell membranes, and related phospholipids called phosphoinositides. Phosphoinositides are involved in the regulation of diverse cellular processes, including cellular proliferation, survival, cytoskeletal organization, vesicle trafficking, glucose transport, and platelet function. Fruman et al., “Phosphoinositide Kinases,” Ann. Review. Biochem. 1998, 67, 481-507. Phosphorylated derivatives of phosphatidylinositol regulate cytoskeletal functions, membrane trafficking, and receptor signaling by recruiting protein complexes to cell and endosomal membranes.

FYVE-type finger-containing phosphoinositide kinase (PIKfyve; also known as phosphatidylinositol-3-phosphate 5-kinase type III or PIPKIII) is a ubiquitously expressed PIK with both lipid and protein kinase activity. In its capacity as a lipid kinase, the enzyme phosphorylates the D-5 position in endosomal phosphatidylinositol and phosphatidylinositol-3-phosphate (PI3P) to generate the corresponding 5-phosphate phospholipid analogs. Shisheva et al., Cell Biol. Int. 2008, 32(6), 591. PI3P is found in cell membranes with roles in protein trafficking, protein degradation, and autophagy. Nascimbeni et al., FEBS J. 2017, 284, 1267-1278. PIKfyve regulates endomembrane homeostasis and plays a role in the biogenesis of endosome carrier vesicles from early endosomes. The enlarged endosome/lysosome structure was observed in cells expressing PIKfyve dominant negative or siRNA. Ikonomov et al., J. Biol. Chem. 2001, 276(28), 26141-26147; Rutherford et al., J. Cell Sci. 2006, 119, 3944-3957. Inhibition of PIKfyve activity increases levels of PI3P, stimulating autophagy and improving motor neuron health. Phosphorylated inositides produced by PIKfyve are localized in various cellular membranes and organelles, consistent with the various PIKfyve functions of endolysosomal transport, endomembrane homeostasis, and biogenesis of endosome carrier vesicles (ECV)/multivesicular bodies (MVB) from early endosomes. Further, PIKfyve is required for endocytic-vacuolar pathway and nuclear migration. Thus, PIKfyve helps maintain proper morphology of the endosome and lysosome.

In mammalian cells, PI3P levels are regulated by the reciprocal activities of PIKfyve and the phosphatase FIG. 4 phosphoinositide 5-phosphatase (FIG. 4). Zolov et al., “In vivo, Pikfyve generates PI(3,5)P2, which serves as both a signaling lipid and the major precursor for PI5P,” Proc. Natl. Acad. Sci. USA 2012, 109(43), 17472-17477. Normally, FIG. 4 is localized on the cytoplasmic surface of endolysosomal vesicles in a complex. Inhibition of PIKfyve would mimic overexpression of FIG. 4, thereby increasing levels of PI3P, stimulating autophagy, and improving motor neuron health. Numerous diseases are correlated with FIG. 4 deficiencies, such as deleterious FIG. 4 mutations or diminished FIG. 4 function, and are therefore suitable as target diseases for treatment with PIKfyve inhibitors, including amyotrophic lateral sclerosis (ALS), primary lateral sclerosis (PLS), Charcot-Marie-Tooth (including type 4J (CMT4J)), and Yunis-Varon syndrome. Mutations in PIKfyve are associated with corneal fleck dystrophy, an autosomal dominant disorder characterized by numerous white flecks in all layers of the corneal stroma.

Exemplary diseases associated with FIG. 4 deficiencies are amyotrophic lateral sclerosis (ALS), primary lateral sclerosis (PLS), Charcot-Marie-Tooth (including type 4J (CMT4J)), Yunis-Varon syndrome, polymicrogyria (including polymicrogyria with seizures), temporo-occipital polymicrogyria, Pick's disease, Parkinson's disease, Parkinson's disease with Lewy bodies, dementia with Lewy bodies, Lewy body disease, frontotemporal dementia, diseases of neuronal nuclear inclusions of polyglutamine and intranuclear inclusion bodies, disease of Marinesco and Hirano bodies, Alzheimer's disease, neurodegeneration, spongiform neurodegeneration, autophagy, peripheral neuropathy, leukoencephalopathy, motor neuropathy, sensory neuropathy. Bharadwaj et al., Hum. Mol. Genet. 2016, 25(4), 682-692.

PIKfyve inhibitors are useful in a range of neurological disorders, such as tauopathies (including but not limited to Alzheimer's disease, progressive supranuclear palsy, corticobasal syndrome, frontotemporal dementias, and chronic traumatic encephalopathy), traumatic brain injury (TBI), cerebral ischemia, ALS, frontotemporal dementia (FTD), Guillain-Barré Syndrome, chronic inflammatory demyelinating polyneuropathy, multiple sclerosis, CMT, lysosomal storage diseases (including but not limited to Fabry's disorder, Gaucher's disorder, Niemann Pick C, Tay-Sachs, and Mucolipidosis type IV), as well as several types of neuropathies. Other therapeutic targets for intervention with PIKfyve inhibitors include Huntington's disease and psychiatric disorders (such as ADHD, schizophrenia, mood disorders including but not limited to major depressive disorder, bipolar disorder I, and bipolar disorder II). Gardiner et al., “Prevalence of carriers of intermediate and pathological polyglutamine disease-associated alleles among large population-based cohorts,” JAMA Neurol. 2019, 76(6), 650-656; PCT Publ. No. WO2016/210372; US Publ. No. US2018/0161335.

In some aspects, the compounds described herein inhibit PI3K, including various isoforms of PI3K such as PI3Kα, β, δ, and/or γ. PI3K, also known as phosphoinositide 3-kinase or phosphatidylinositol 3-kinase, is a family of enzymes involved in cellular functions such as cell growth, proliferation, differentiation, motility, survival, and intracellular trafficking. PI3K inhibitors are useful as potential therapeutics in a range of disease states including, for example, central nervous system diseases.

### SUMMARY

In a first aspect, this disclosure is directed to a compound of Formula (I):

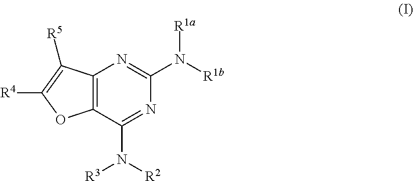

wherein:
R^{1a} and R^{1b} taken together with the nitrogen to which they are attached form:

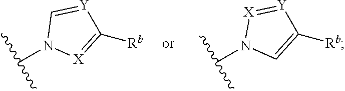

wherein X and Y are independently N or CR^{a};
   wherein R^{a} is H or C₁₋₄alkyl; and
   R^{b} is phenyl, monocyclic cycloalkyl, monocyclic heterocyclyl, monocyclic heterocycloalkyl, or monocyclic heteroaryl, each optionally substituted with one, two, or three R^{d} substituents;
or R^{1a} is H or C₁₋₄alkyl; and R^{1b} is a heteroaryl optionally substituted with R^{c};
   wherein R^{c} is C₁₋₄alkyl, phenyl, —C₁₋₄alkyl-phenyl, monocyclic cycloalkyl, —C₁₋₄alkyl-(monocyclic cycloalkyl), monocyclic heterocyclyl, monocyclic heterocycloalkyl, monocyclic heteroaryl, or —C₁₋₄alkyl-(monocyclic heteroaryl), wherein each alkyl, phenyl, cycloalkyl, heterocyclyl, heterocycloalkyl or heteroaryl is optionally substituted with one, two, or three R^{d} substituents;
   wherein each R^{d} substituent is independently C₁₋₄alkyl, C₁₋₄alkenyl, C₁₋₄alkynyl, —O—C₁₋₄alkyl, halo, cyano, nitro, azido, C₁₋₄haloalkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, ═NOR^{g}, —NR^{g}S(═O)₁₋₂R^{h}, —NR^{g}S(═O)₁₋₂NR^{g}R^{h}, ═NSO₂R^{g}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, or —S(═O)₁₋₂NR^{g}R^{h};
      wherein R^{g} and R^{h} are each independently H or C₁₋₄alkyl;
each of R² and R³ is independently chosen from H, C₁₋₄alkyl, cycloalkyl, C₁₋₄alkylcycloalkyl, heterocyclyl, heterocycloalkyl, and heteroaryl each optionally substituted with one, two, or three R^{j} substituents; or R² and R³ taken together with the nitrogen to which they are attached form a heterocyclyl, optionally substituted with one, two, three, or four R^{j} substituents, or further wherein any of the hydrogens bonded to carbon atoms are optionally replaced by deuterium;
   wherein each Rⱼ substituent is independently C₁₋₄alkyl, —OH, —NR^{k}R^{l}, halo, C₁₋₄haloalkyl, —O—, C₁₋₄alkyl, or —O—C₁₋₄-haloalkyl;
      where R^{k} and R^{l} are each independently H or C₁₋₄alkyl;
R⁴ is H, halo, —C(O)OH, C₁₋₄alkylNR^{x}R^{y}, or —C(O)NR^{x}R^{y}, or is a cycloalkyl, heterocyclyl, heterocycloalkyl, phenyl or heteroaryl, wherein each cycloalkyl, heterocyclyl, heterocycloalkyl, phenyl or heteroaryl is optionally substituted with one, two, or three R^{z} substituents;
   wherein R^{x} is H or C₁₋₄alkyl and R^{y} is H, C₁₋₄alkyl, —O—C₁₋₄alkyl, —SO₂—R^{r}, C₁₋₄alkyl-SO₂—R^{r} monocyclic cycloalkyl, —C₁₋₄alkyl(monocyclic cycloalkyl), monocyclic heterocyclyl, or monocyclic heterocycloalkyl, each optionally substituted with one, two, or three R^{c} substituents;
   or R^{x} and R^{y} taken together with the nitrogen to which they are attached form a monocyclic heterocyclyl, optionally substituted with C₁₋₄alkyl or —OC₁₋₄alkyl; and
   each R^{z} substituent is independently C₁₋₄alkyl, halo, —NR^{p}R^{q}, —C(O)NR^{p}R^{q}, —OH, or —OC₁₋₄alkyl, wherein each alkyl is optionally substituted with —NR^{m}Rⁿ;
      wherein R^{m} and Rⁿ are each independently H, C₁₋₄alkyl, C(O)C₁₋₂alkyl, C(O)C₁₋₂haloalkyl, C(O)C₁₋₂alkenyl, or R^{m} and Rⁿ taken together with the nitrogen to which they are attached form a monocyclic heterocycloalkyl, optionally substituted with one or two R^{o} substituents;
   wherein each R^{o} substituent is independently C₁₋₄alkyl, —OH, —OC₁₋₄alkyl, halo, cyano, methylsulfonyl, —NR^{p}R^{q}, or —C(O)NR^{p}R^{q};
      wherein R^{p} and R^{q} are each independently H, C₁₋₄alkyl, C₁₋₄alkylNH₂, C₁₋₄alkylNH(C₁₋₄alkyl), or C₁₋₄alkylN(C₁₋₄alkyl)₂;
   wherein each R^{r} substituent is independently C₁₋₄alkyl or NR^{p}R^{q}; and
R⁵ is H, C₁₋₄alkyl, halo, —OH, or —OC₁₋₄alkyl;
   or a pharmaceutically acceptable salt or prodrug thereof.

In a second aspect, this disclosure is directed to a pharmaceutical composition comprising a compound of Formula (I) (or any of the embodiments thereof described herein), or a pharmaceutically acceptable salt or prodrug thereof, and a pharmaceutically acceptable excipient.

In a third aspect, this disclosure is directed to a method of inhibiting PIKfyve and/or a PI3 kinase in a subject in need thereof comprising administering to the subject an effective amount of a compound of Formula (I) (or any of the embodiments thereof described herein), or a pharmaceutically acceptable salt or prodrug thereof, or a pharmaceutical composition comprising a compound of Formula (I) (or any of the embodiments thereof described herein), or a pharmaceutically acceptable salt or prodrug thereof.

In a fourth aspect, this disclosure is directed to a method of treating a neurological disease treatable by inhibition of PIKfyve and/or a PI3 kinase activity in a subject in need thereof comprising administering to the subject in need thereof a compound of Formula (I) (or any of the embodiments thereof described herein), or a pharmaceutically acceptable salt or prodrug thereof, or a pharmaceutical composition comprising a compound of Formula (I) (or any of the embodiments thereof described herein), or a pharmaceutically acceptable salt or prodrug thereof.

In a fourth aspect, the disclosure is directed a compound of Formula (I) (and any embodiments thereof described herein), or a pharmaceutically acceptable salt or prodrug thereof, for use as a medicament. In one embodiment, the use of the compound of Formula (I) and/or a pharmaceutically acceptable salt or prodrug thereof is for treating a disease treatable by inhibition of PIKfyve and/or a PI3 kinase or associated with PIKfyve and/or PI3 kinase activity.

In a fifth aspect is the use of a compound of Formula (I) (or any of the embodiments thereof described herein), or a pharmaceutically acceptable salt or prodrug thereof, in the manufacture of a medicament for treating a disease in a mammal in which PIKfyve or PI3K contributes to the pathology and/or symptoms of the disease.

### DETAILED DESCRIPTION

Unless otherwise stated, the following terms used in the specification and claims are defined for the purposes of this disclosure and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkylsulfonyl” means a —SO₂R radical where R is alkyl as defined above, e.g., methylsulfonyl, ethylsulfonyl, and the like.

“Amino” means a —NH₂.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with an alkoxy group, (in one embodiment one or two alkoxy groups), as defined above, e.g., 2-methoxyethyl, 1-, 2-, or 3-methoxypropyl, 2-ethoxyethyl, and the like.

“Alkoxycarbonyl” means a —C(O)OR radical where R is alkyl as defined above, e.g., methoxycarbonyl, ethoxycarbonyl, and the like.

“Acyl” means a —COR radical where R is alkyl, haloalkyl, or cycloalkyl, e.g., acetyl, propionyl, cyclopropylcarbonyl, and the like. When R is alkyl, the radical is also referred to herein as alkylcarbonyl.

“Cycloalkyl” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like.

“Carboxy” means —COOH.

“Halo” means fluoro, chloro, bromo, or iodo; in one embodiment fluoro or chloro.

“Haloalkyl” means alkyl radical as defined above, which is substituted with one or one to five halogen atoms (in one embodiment fluorine or chlorine,) including those substituted with different halogens, e.g., —CH₂Cl, —CF₃, —CHF₂, —CH₂CF₃, —CF₂CF₃, —CF(CH₃)₂, and the like. In C_{x-y}-haloalkyl, “C_{x-y}” means the number of carbon atoms in the alkyl group ranges from x to y. When the alkyl is substituted with only fluoro, it can be referred to in this disclosure as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it can be referred to in this disclosure as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl. Further examples include, but are not limited to, 2-hydroxyethyl, 2,3-dihydroxypropyl, and 1-(hydroxymethyl)-2-hydroxyethyl.

“Heterocyclyl” means a saturated or unsaturated monovalent monocyclic or bi-cyclic group (fused bi-cyclic or bridged bi-cyclic) of 4 to 10 ring atoms in which one or two ring atoms are heteroatom selected from N, O, and S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, oxetanyl, pyrrolidino, piperidino, homopiperidino, 2-oxopyrrolidinyl, 2-oxopiperidinyl, morpholino, piperazino, tetrahydropyranyl, thiomorpholino, hexahydropyrrolo[1,2-a]pyrazin-6(2H)-one-yl, tetrahydro-1H-oxazolo[3,4-a]pyrazin-3(5H)-one-yl, 5,6,7,8-tetrahydro-[1,2,4]triazolo[4,3-a]pyrazine-yl, 3-oxa-8-azabicyclo[3.2.1]octane-yl, and the like. When the heterocyclyl ring is unsaturated it can contain one or two ring double bonds provided that the ring is not aromatic.

“Heterocyclylalkyl” and “heterocycloalkyl” mean an -(alkylene)-R radical where R is heterocyclyl ring as defined above e.g., tetraydrofuranylmethyl, piperazinylmethyl, morpholinylethyl, and the like.

“Heterocycloamino” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C provided that at least one of the ring atoms is N. Additionally, one or two ring carbon atoms in the heterocycloamino ring can optionally be replaced by a —CO— group. When the heterocycloamino ring is unsaturated it can contain one or two ring double bonds provided that the ring is not aromatic.

“Heterocycloaminoalkyl” means a -(alkylene)-R radical where R is heterocycloamino as described above.

“Heteroaryl” means a monovalent monocyclic or bicyclic aromatic radical of 5 to 10 ring atoms where one or more, (in one embodiment one, two, or three), ring atoms are heteroatom selected from N, O, and S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, pyrazolopyridinyl, indazolyl, furopyrimidinyl, and the like.

“Mammal” as used herein means domesticated animals (such as dogs, cats, and horses), and humans. In one embodiment, mammal is a human.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Oxo” means an ═(O) group and “carbonyl” means a >C(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

“Treating” or “treatment” of a disease includes:

(1) preventing the disease, e.g., causing the clinical symptoms of the disease not to develop in a mammal that may be exposed to or predisposed to the disease but does not yet experience or display symptoms of the disease;

(2) inhibiting the disease, e.g., arresting or reducing the development of the disease or its clinical symptoms; or

(3) relieving the disease, e.g., causing regression of the disease or its clinical symptoms.

A “therapeutically effective amount” means the amount of a compound of Formula (I) (or any of the embodiments thereof described herein), that, when administered to a mammal for treating a disease, is sufficient to treat the disease. The “therapeutically effective amount” will vary depending on the compound, the disease and its severity and the age, weight, etc., of the mammal to be treated.

The compounds described herein may in some cases exist as diastereomers, enantiomers, or other stereoisomeric forms. All chiral, diastereomeric, racemic forms, as individual forms and mixtures thereof, are within the scope of this disclosure, unless the specific stereochemistry or isomeric form is specifically indicated. Compounds of the present disclosure containing an asymmetrically substituted atom may be isolated in optically active, optically enriched, optically pure, or racemic forms. It is well known in the art how to prepare optically active forms, such as by resolution of materials. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

Certain compounds of Formula (I) (or any of the embodiments thereof described herein) and/or a pharmaceutically acceptable salt or prodrug thereof can exist as tautomers and/or geometric isomers. All possible tautomers and cis and trans isomers, as individual forms and mixtures thereof, are within the scope of this disclosure. For example, pyrazole tautomers as shown below are equivalent structures. The depiction of one such structure is intended to encompass both structures.

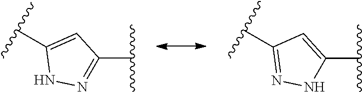 [SMILES: *c1cc(*)[nH]n1 ; *c1cc(*)[nH]n1]

Additionally, as used herein the term alkyl includes all the possible isomeric forms of said alkyl group albeit only a few examples are set forth. Furthermore, when the cyclic groups such as heteroaryl, heterocyclyl are substituted, they include all the positional isomers.

Pharmaceutically acceptable salts of the compounds of Formula (I) (or any of the embodiments thereof described herein) are within the scope of this disclosure. In addition, the compounds described herein include hydrates and solvates of the compounds or pharmaceutically acceptable salts thereof.

The present disclosure also includes the prodrugs of compounds of Formula (I) (or any of the embodiments thereof described herein) and/or a pharmaceutically acceptable salt or prodrug thereof. The term prodrug is intended to represent covalently bonded carriers, which are capable of releasing the active ingredient of Formula (I) (or any of the embodiments thereof described herein) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) (or any of the embodiments thereof described herein) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) or phosphonates (e.g., —OP(═O)(OH)₂) of hydroxy or amino functional groups in compounds of Formula (I)), amides (e.g., trifluoroacetylamino, acetylamino, and the like), and the like. Prodrugs of compounds of Formula (I) (or any of the embodiments thereof described herein) and/or a pharmaceutically acceptable salt or prodrug thereof are also within the scope of this disclosure.

The present disclosure also includes polymorphic forms (amorphous as well as crystalline) and deuterated forms of compounds of Formula (I) (or any of the embodiments thereof described herein) and/or a pharmaceutically acceptable salt or prodrug thereof.

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Rccent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

In one aspect is a compound of Formula (I):

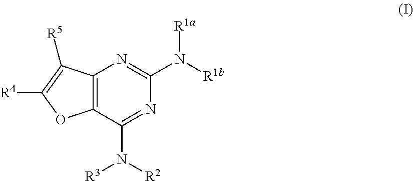

wherein:
R^{1a} and R^{1b} taken together with the nitrogen to which they are attached form:

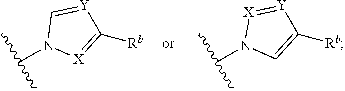

wherein X and Y are independently N or CR^{a};
   wherein R^{a} is H or C₁₋₄alkyl; and
   R^{b} is phenyl, monocyclic cycloalkyl, monocyclic heterocyclyl, monocyclic heterocycloalkyl, or monocyclic heteroaryl, each optionally substituted with one, two, or three R^{d} substituents;
or R^{1a} is H or C₁₋₄alkyl; and R^{1b} is a heteroaryl optionally substituted with R^{c};
   wherein R^{c} is C₁₋₄alkyl, phenyl, —C₁₋₄alkyl-phenyl, monocyclic cycloalkyl, —C₁₋₄alkyl-(monocyclic cycloalkyl), monocyclic heterocyclyl, monocyclic heterocycloalkyl, monocyclic heteroaryl, or —C₁₋₄alkyl-(monocyclic heteroaryl), wherein each alkyl, phenyl, cycloalkyl, heterocyclyl, heterocycloalkyl or heteroaryl is optionally substituted with one, two, or three R^{d} substituents;
   wherein each R^{d} substituent is independently C₁₋₄alkyl, C₁₋₄alkenyl, C₁₋₄alkynyl, —O—C₁₋₄alkyl, halo, cyano, nitro, azido, C₁₋₄haloalkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, ═NOR^{g}, —NR^{g}S(═O)₁₋₂R^{h}, —NR^{g}S(═O)₁₋₂NR^{g}R^{h}, ═NSO₂R^{g}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, or —S(═O)₁₋₂NR^{g}R^{h};
      wherein R^{g} and R^{h} are each independently H or C₁₋₄alkyl;
each of R² and R³ is independently chosen from H, C₁₋₄alkyl, cycloalkyl, C₁₋₄alkylcycloalkyl, heterocyclyl, heterocycloalkyl, and heteroaryl, each optionally substituted with one, two, or three R^{j} substituents; or R² and R³ taken together with the nitrogen to which they are attached form a heterocyclyl, optionally substituted with one, two, three, or four R^{j} substituents, or further wherein any of the hydrogens bonded to carbon atoms are optionally replaced by deuterium;
   wherein each R^{j} substituent is independently C₁₋₄alkyl, —OH, —NR^{k}R^{l}, halo, C₁₋₄haloalkyl, —O—C₁₋₄alkyl, or —O—C₁₋₄-haloalkyl;
      where R^{k} and R^{l} are each independently H or C₁₋₄alkyl;
R⁴ is H, halo, —C(O)OH, C₁₋₄alkylNR^{x}R^{y}, or —C(O)NR^{x}R^{y}, or is a cycloalkyl, heterocyclyl, heterocycloalkyl, phenyl or heteroaryl, wherein each cycloalkyl, heterocyclyl, heterocycloalkyl, phenyl or heteroaryl is optionally substituted with one, two, or three R^{z} substituents;
   wherein R^{x} is H or C₁₋₄alkyl and R^{y} is H, C₁₋₄alkyl, —O—C₁₋₄alkyl, —SO₂—R^{r}, C₁₋₄alkyl-SO₂—R^{r} monocyclic cycloalkyl, —C₁₋₄alkyl(monocyclic cycloalkyl), monocyclic heterocyclyl, or monocyclic heterocycloalkyl, each optionally substituted with one, two, or three R^{o} substituents;
   or R^{x} and R^{y} taken together with the nitrogen to which they are attached form a monocyclic heterocyclyl, optionally substituted with C₁₋₄alkyl or —OC₁₋₄alkyl; and
   each R^{z} substituent is independently C₁₋₄alkyl, halo, —NR^{p}R^{q}, —C(O)NR^{p}R^{q}, —OH, or —OC₁₋₄alkyl, wherein each alkyl is optionally substituted with —NR^{m}Rⁿ;
      wherein R^{m} and Rⁿ are each independently H, C₁₋₄alkyl, C(O)C₁₋₂alkyl, C(O)C₁₋₂haloalkyl, C(O)C₁₋₂alkenyl, or R^{m} and Rⁿ taken together with the nitrogen to which they are attached form a monocyclic heterocycloalkyl, optionally substituted with one or two R^{o} substituents;
   wherein each R^{o} substituent is independently C₁₋₄alkyl, —OH, —OC₁₋₄alkyl, halo, cyano, methylsulfonyl, —NR^{p}R^{q}, or —C(O)NR^{p}R^{q};
      wherein R^{p} and R^{q} are each independently H, C₁₋₄alkyl, C₁₋₄alkylNH2, C₁₋₄alkylNH(C₁₋₄alkyl), or C₁₋₄alkylN(C₁₋₄alkyl)₂;
   wherein each R^{r} substituent is independently C₁₋₄alkyl or NR^{P}R^{q}; and
R⁵ is H, C₁₋₄alkyl, halo, —OH, or —OC₁₋₄alkyl;
   or a pharmaceutically acceptable salt or prodrug thereof.

In some embodiments, R^{1a} and R^{1b} are taken together with the nitrogen to which they are attached to form

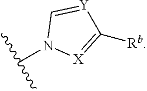

In some embodiments, R^{1a} and R^{1b} are taken together with the nitrogen to which they are attached to form

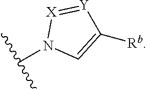

In some embodiments, X is N and Y is CR^{a}. In some embodiments, X is CR^{a} and Y is N. In some embodiments, X is N and Y is N. In some embodiments, R^{a} is H, methyl, ethyl, propyl, isopropyl, butyl, isobutyl, sec-butyl, or tert-butyl. In some embodiments, R^{a} is H or methyl. In some embodiments, R^{a} is H.

In some embodiments, R^{b} is optionally substituted phenyl. In some embodiments, R^{b} is optionally substituted monocyclic heteroaryl. In some embodiments, R^{b} is optionally substituted pyrrolyl, imidazolyl, pyrazolyl, triazolyl, tetrazolyl, furanyl, oxazolyl, isoxazolyl, thiazolyl, isothiazolyl, pyridinyl, pyrimidinyl, pyrazinyl, or pyridazinyl. In some embodiments, R^{b} is optionally substituted pyridinyl or pyrimidinyl. In some embodiments, R^{b} is optionally substituted pyridinyl. In some embodiments, R^{b} is phenyl. In some embodiments, R^{b} is o-, m-, or p-tolyl. In some embodiments, R^{b} is optionally substituted with one or two R^{d} substituents. In some embodiments, R^{b} is optionally substituted with one R^{d} substituent. In some embodiments, R^{b} is methylpyridinyl, phenyl, tolyl, chlorophenyl, bromophenyl, or methoxyphenyl.

In some embodiments, R^{1a} is H or C₁₋₄alkyl; and R^{1b} is a 5-membered N-containing heteroaryl optionally substituted with R^{c}. In some embodiments, R^{1a} is H. In some embodiments, R^{1a} is C₁₋₄alkyl. In some embodiments, R^{1a} is methyl.

In some embodiments, R^{1b} is pyrrolyl, pyrazolyl, imidazolyl, triazolyl, oxazolyl, isoxazolyl, thiazolyl, isothiazolyl, oxadiazolyl, thiadiazolyl, pyrazolopyridinyl, or indazolyl, each optionally substituted with R^{c}. In some embodiments, R^{1b} is pyrazolyl, imidazolyl, oxazolyl, oxadiazolyl or isoxazolyl, each optionally substituted with R^{c}. In some embodiments, R^{1b} is pyrazolyl, optionally substituted with R^{c}. In some embodiments, R^{1b} is

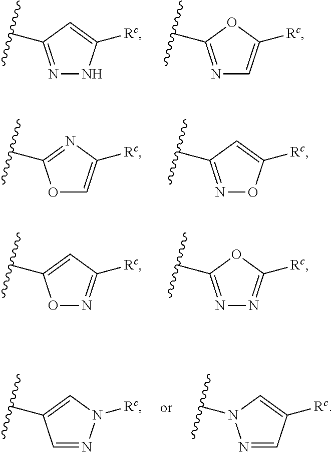

In some embodiments, R^{1b} is

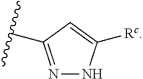

In some embodiments, R^{c} is optionally substituted C₁₋₄alkyl. In some embodiments, R^{c} is methyl, ethyl, isopropyl, or trifluoromethyl. In some embodiments, R^{c} is optionally substituted phenyl. In some embodiments, R^{c} is phenyl or o-, m-, p-tolyl, fluorophenyl, methoxyphenyl, or trifluoromethoxyphenyl. In some embodiments, R^{c} is phenyl. In some embodiments, R⁰ is optionally substituted monocyclic cycloalkyl. In some embodiments, R^{c} is optionally substituted cyclopropyl, cyclobutyl, cyclopentyl, or cyclohexyl. In some embodiments, R^{c} is optionally substituted cyclopropyl. In some embodiments, R^{c} is optionally substituted monocyclic heterocycloalkyl. In some embodiments, R^{c} is optionally substituted cyclopropylmethyl, cyclobutylmethyl, cyclopentylmethyl, or cyclohexylmethyl. In some embodiments, R^{c} is optionally substituted monocyclic heterocyclyl. In some embodiments, R^{c} is optionally substituted pyrrolidinyl, tetrahydrofuranyl, piperidinyl, morpholinyl, or piperazinyl. In some embodiments, R^{c} is optionally substituted monocyclic heteroaryl. In some embodiments, R^{c} is optionally substituted pyrrolyl, imidazolyl, pyrazolyl, triazolyl, tetrazolyl, furanyl, thiophenyl, oxazolyl, isoxazolyl, thiazolyl, isothiazolyl, pyridinyl, pyrimidinyl, pyrazinyl, or pyridazinyl. In some embodiments, R^{c} is optionally substituted pyrazole, thiophenyl, imidazolyl, pyridinyl, or pyrimidinyl. In some embodiments, R^{c} is optionally substituted pyrazolyl. In some embodiments, R^{c} is optionally substituted pyridinyl. In some embodiments, R^{c} is methylpyridinyl. In some embodiments, R^{c} is optionally substituted —C₁₋₄alkyl-phenyl, —C₁₋₄alkyl-(monocyclic cycloalkyl), monocyclic heterocycloalkyl, or —C₁₋₄alkyl-(monocyclic heteroaryl). In some embodiments, R^{c} is optionally substituted benzyl, —CH₂-(monocyclic cycloalkyl), —CH₂-(monocyclic heterocycloalkyl), or —CH₂-(monocyclic heteroaryl). In some embodiments, R^{c} is optionally substituted benzyl or —CH₂-(monocyclic cycloalkyl), such as —CH₂-cyclopropyl. In some embodiments, each R^{c} is optionally substituted with one or two R^{d} substituents.

In some embodiments, each R^{d} substituent is independently C₁₋₄alkyl, C₁₋₄alkenyl, C₁₋₄alkynyl, —O—C₁₋₄alkyl, halo, cyano, nitro, azido, C₁₋₄haloalkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, ═NOR^{g}, —NR^{g}S(═O)₁₋₂R^{h}, —NR^{g}S(═O)₁₋₂NR^{g}R^{h}, ═NSO₂R^{g}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, or —S(═O)₁₋₂NR^{g}R^{h}. In some embodiments, each R^{d} substituent is independently C₁₋₄alkyl, —O—C₁₋₄alkyl, C₁₋₄haloalkyl, or halo. In some embodiments, each R^{d} substituent is independently methyl, ethyl, isopropyl, —CF₃, —OCH₃, —OCF₃, or fluoro.

In some embodiments, R^{g} and R^{h} are each independently H or methyl.

In some embodiments, each of R² and R³ are independently selected from H, pyrrolidinyl, piperidinyl, piperazinyl, and imidazolyl, wherein each pyrrolidinyl, piperidinyl, piperazinyl, and imidazolyl is optionally substituted with one R^{j} substituent.

In some embodiments, R² and R³ taken together with the nitrogen to which they are attached form pyrrolidinyl, piperidinyl, piperazinyl, imidazolyl, morpholino, thiomorpholino, or thiomorpholino-1,1-dioxide, each optionally substituted with one, two, three, or four R^{j} substituents. In some embodiments, R² and R³ taken together with the nitrogen to which they are attached morpholino, imidazolyl, or piperazinyl, optionally substituted with one, two, three, or four R^{j} substituents. In some embodiments, R² and R³ taken together with the nitrogen to which they are attached form 2,2,6,6-tetrafluoro-morpholino morpholino-3-one, morpholino-3-one, piperazinyl-2-one, piperazinyl-3-one, thiomorpholino-1,1-dioxide.

In some embodiments, each R^{j} substituent is independently methyl, oxo, hydroxy, —OCH₃, NH₂, halo, —CF₃, or —OCF₃.

In some embodiments, R² and R³ taken together with the nitrogen to which they are attached form morpholino in which 1 to 8 hydrogens are replaced with deuterium.

In some embodiments, R^{k} and R^{l} are each independently H or methyl.

In some embodiments, R⁴ is H. In some embodiments, R⁴ is chloro.

In some embodiments, R⁴ is optionally substituted phenyl. In some embodiments, R⁴ is optionally substituted heteroaryl. In some embodiments, R⁴ is optionally substituted monocyclic heteroaryl. In some embodiments, R⁴ is optionally substituted pyrrolyl, imidazolyl, pyrazolyl, triazolyl, tetrazolyl, furanyl, oxazolyl, isoxazolyl, thiazolyl, isothiazolyl, pyridinyl, pyrimidinyl, pyrazinyl, or pyridazinyl. In some embodiments, R⁴ is optionally substituted pyridinyl or pyrimidinyl.

In some embodiments, R⁴ is

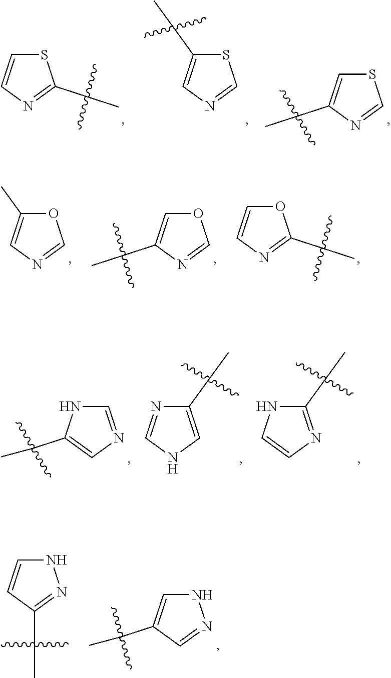

each optionally substituted with 1 or 2 R^{z} groups.

In some embodiments, R⁴ is optionally substituted pyridinyl. In some embodiments, R⁴ is pyridinyl. In some embodiments, R⁴ is 4-pyridyl, 3-pyridyl, or 2-pyridyl. In some embodiments, R⁴ is 4-pyridyl. In some embodiments, R⁴ is optionally substituted with one or two R^{z} substituents. In some embodiments, R⁴ is phenyl or pyridyl, each optionally substituted with one or two substituents selected from C₁₋₄alkyl, —CF₃, fluoro, chloro, —OCH₃, and —OCF₃.

In some embodiments, R⁴ is heterocyclyl, optionally substituted with one or two R^{z} substituents. In some embodiments, R⁴ is pyrrolidinyl, piperidinyl, piperazinyl, morpholino, or thiomorpholino, optionally substituted with one or two R^{z} substituents. In some embodiments, R⁴ is pyrrolidinyl, or piperazinyl, optionally substituted with one C₁₋₄alkyl. In some embodiments, R⁴ is optionally substituted pyrazolyl. In some embodiments, R⁴ is optionally substituted with one or two R^{z} substituents. In some embodiments, R⁴ is optionally substituted with one R^{z} substituent. In some embodiments, R⁴ is 3-methyl-1H-pyrazol-5-yl, 3-methylisothiazol-5-yl, 2-methyl-1H-imidazol-5-yl, 1-methyl-pyrazol-4-yl, 1-methylpyrazol-3-yl, 1-((1-acetamido)-eth-2-yl)-5-methyl-pyrazol-3-yl, 1-((1-chloromethylamido)-eth-2-yl)-5-methyl-pyrazol-3-yl, 1-((1-acrylamido)-eth-2-yl)-5-methyl-pyrazol-3-yl, thiazol-2-yl, pyrazol-4-yl, pyrazol-1-yl, oxazol-2-yl, or 3-(1-N,N-dimethyl-eth-2-yl)-4-methyl-pyrazol-1-yl.

In some embodiments, R⁴ is heterocycloalkyl, optionally substituted with one or two R^{z} substituents. In some embodiments, R⁴ is pyrrolidinylmethyl, piperidinylmethyl, piperazinylmethyl, morpholinomethyl, or thiomorpholinomethyl, optionally substituted with one or two R^{z} substituents.

In some embodiments, R⁴ is C₁₋₄alkylNR^{x}R^{y}. In some embodiments, R⁴ is CH₂NR^{x}R^{y}. In some embodiments, R⁴ is —C(O)NR^{x}R^{y}.

In some embodiments, R^{x} is H. In some embodiments, R^{x} is methyl or ethyl, optionally substituted with one, two, or three R^{o} substituents. In some embodiments, R^{x} is methyl.

In some embodiments, R^{y} is H, methyl, ethyl, methyoxy, or methoxyethyl. In some embodiments, R^{y} is H. In some embodiments, R^{y} is C₁₋₄alkyl, optionally substituted with one, two, or three R^{o} substituents. In some embodiments, R^{y} is methyl, ethyl, propyl, or isopropyl, each optionally substituted with one, two, or three R^{o} substituents. In some embodiments, R^{y} is methyl, ethyl, or methoxyethyl. In some embodiments, R^{y} is methoxy. In some embodiments, R^{y} is —SO₂—R^{r} or C₁₋₄alkyl-SO₂—R^{r}. R^{y} is —SO₂—R^{r}, C₁₋₄alkyl-SO₂—R^{r}; and R^{r} is CH₃ or NH₂, NHCH₃, or N(CH₃)₂. In some embodiments, R^{y} is —SO₂-methyl, C₂₋₄alkyl-SO₂—N(CH₃)₂. In some embodiments, R^{y} is —SO₂-methyl. In some embodiments, R^{y} is monocyclic cycloalkyl or —C₁₋₂alkyl(monocyclic cycloalkyl), each optionally substituted with one, two, or three R^{o} substituents. In some embodiments, R^{y} is monocyclic cycloalkyl, optionally substituted with one, two, or three R^{o} substituents. In some embodiments, R^{y} is cyclopropyl, cyclobutyl, cyclopentyl, or cyclohexyl, each optionally substituted with one, two, or three R^{c} substituents. In some embodiments, R^{y} is cyclopropyl. In some embodiments, R^{y} is cyclopropyl, cyclobutyl, cyclopentyl, cyclopropylmethyl, 1-cyclopropylethyl, 2-cyclopropylethyl, cyclobutylmethyl, or cyclopentylmethyl. In some embodiments, R^{y} is monocyclic heterocycloalkyl, optionally substituted with one, two, or three R^{o} substituents. In some embodiments, R^{y} is optionally substituted azetidinyl, oxetanyl, pyrrolidinyl, piperidinyl, morpholinyl, piperazinyl, azepanyl, azocanyl, tetrahydrofuranyl, or tetrahydropyranyl, optionally substituted with methyl. In some embodiments, R^{y} is optionally substituted azetidinyl, pyrrolidinyl, piperidinyl, morpholinyl, or piperazinyl. In some embodiments, R^{y} is monocyclic heterocycloalkyl, optionally substituted with one, two, or three R^{o} substituents. In some embodiments, R^{y} is optionally substituted azetidinylmethyl, oxetanylmethyl, pyrrolidinylmethyl, piperidinylmethyl, morpholinylmethyl, or piperazinylmethyl, optionally substituted with methyl.

In some embodiments, R^{x} and R^{y} is H and the other is —CH₃. In some embodiments, both of R^{x} and R^{y} is H. In some embodiments, both of R^{x} and R^{y} is —CH₃.

In some embodiments, R^{x} and R^{y} taken together with the nitrogen to which they are attached form a monocyclic heterocyclyl, optionally substituted with C₁₋₄alkyl. In some embodiments, R^{x} and R^{y} are taken together with the nitrogen to which they are attached to form azetidinyl, pyrrolidinyl, piperidinyl, piperazinyl, morpholinyl, or thiomorpholino, each optionally substituted with methyl.

In some embodiments, each R^{z} is independently C₁₋₄alkyl, halo, —OH, or —OC₁₋₄alkyl, wherein each alkyl is optionally substituted with —NR^{m}Rⁿ. In some embodiments, each R^{z} is independently —CH₃, —OH, halo, or —OCH₃. In some embodiments, R^{z} is C₂₋₃alkyl substituted with —NR^{m}Rⁿ. In some embodiments, R^{z} is C₂₋₄alkyl substituted with —NR^{m}Rⁿ or OCH₃. In some embodiments, each R^{z} substituent is independently —NR^{p}R^{q}, —C(O)NR^{p}R^{q}.

In some embodiments, each R^{z} substituent is methyl, ethyl, isopropyl, —CF₃, fluoro, chloro, —OCH₃, —OCF₃, methylamino, ethylamino, propylamino, butylamino, aminomethyl, aminoethyl, aminopropyl, aminobutyl, dimethylamino, dimethylaminomethyl, dimethylaminoethyl, dimethylaminopropyl, dimethylaminobutyl, —C(O)methylamino, —C(O)ethylamino, —C(O)propylamino, —C(O)butylamino, —C(O)dimethylamino, —C(O)dimethylaminomethyl, —C(O)dimethylaminoethyl, —C(O)dimethylaminopropyl, or —C(O)dimethylaminobutyl.

In some embodiments, R^{m} and Rⁿ are each independently H, C₁₋₄alkyl, C(O)CH₃, C(O)CH₂Cl, or C(O)CH₂CH₂. In some embodiments, R^{m} and Rⁿ are each H. In some embodiments, R^{m} and Rⁿ are each methyl. In some embodiments, R^{m} and Rⁿ taken together with the nitrogen to which they are attached form a monocyclic heterocyclyl, optionally substituted with one or two R^{o} substituents. In some embodiments, R^{m} and Rⁿ taken together with the nitrogen to which they are attached form pyrrolidinyl, piperidinyl, piperazinyl, morpholinyl, thiomorpholino, or thiomorpholino-1,1-dioxide, each optionally substituted with one or two R^{o} substituents. In some embodiments, R^{m} and Rⁿ taken together with the nitrogen to which they are attached form pyrrolidinyl, piperidinyl, piperazinyl, or morpholino, each optionally substituted with one or two R^{o} substituents. In some embodiments, R^{m} and Rⁿ taken together with the nitrogen to which they are attached form pyrrolidinyl, piperidinyl, piperazinyl, or morpholinyl, each optionally substituted with methyl.

In some embodiments, each R^{o} substituent is C₁₋₄alkyl. In some embodiments, each R^{o} substituent is —NR^{p}R^{q}. In some embodiments, R^{p} and R^{q} are each independently H or methyl.

In some embodiments, R^{p} and R^{q} are each independently H, methyl, C₁₋₄alkylNH₂, C₁₋₄alkylNHCH₃, or C₁₋₄alkylN(CH₃)₂.

In some embodiments, R⁵ is H, methyl, ethyl, chloro, bromo, fluoro, —OH, or —OCH₃. In some embodiments, R⁵ is H.

In some embodiments, the compound of Formula (I) or the pharmaceutically acceptable salt thereof is a compound of Formula (II):

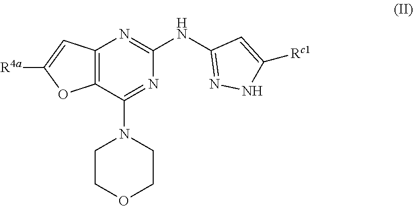

wherein
R^{c1} is phenyl or pyridyl, each optionally substituted with one or two substituents selected from C₁₋₄alkyl, —CF₃, fluoro, chloro, —OCH₃, and —OCF₃; and
R^{4a} is C₁₋₄alkylNR^{x}R^{y} or C(O)NR^{x}R^{y} wherein R^{x} and R^{y} are as defined herein; or phenyl, pyrazolyl, or pyridyl, each optionally substituted with one or two R^{z} groups;
   or a pharmaceutically acceptable salt or prodrug thereof.

In some embodiments, the compound of Formula (I) or the pharmaceutically acceptable salt thereof is a compound of Formula (III):

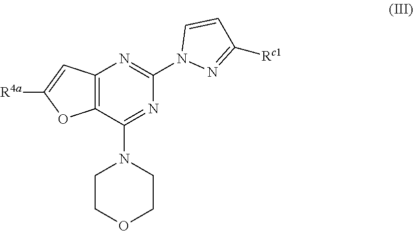

wherein
R^{c1} is phenyl or pyridyl, each optionally substituted with one or two substituents selected from C₁₋₄alkyl, —CF₃, fluoro, chloro, —OCH₃, and —OCF₃; and
R^{4a} is C₁₋₄alkylNR^{x}R^{y} or —C(O)NR^{x}R^{y} wherein R^{x} and R^{y} are as defined herein; or phenyl, pyrazolyl, or pyridyl, each optionally substituted with one or two R^{z} groups;
   or a pharmaceutically acceptable salt or prodrug thereof.

In some embodiments, the compound of Formula (I) or the pharmaceutically acceptable salt thereof is a compound of Formula (IV):

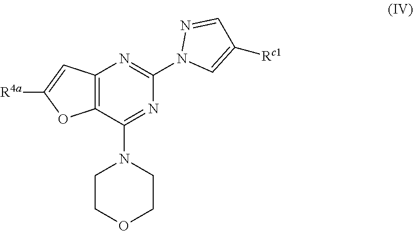

wherein
R^{c1} is phenyl or pyridyl, each optionally substituted with one or two substituents selected from C₁₋₄alkyl, —CF₃, fluoro, chloro, —OCH₃, and —OCF₃; and
R^{4a} is C₁₋₄alkylNR^{x}R^{y} or —C(O)NR^{x}R^{y} wherein R^{x} and R^{y} are as defined herein; or phenyl, pyrazolyl, or pyridyl, each optionally substituted with one or two R^{z} groups;
   or a pharmaceutically acceptable salt or prodrug thereof.

In some embodiments, R^{c1} is phenyl or pyridyl, each optionally substituted with methyl, —CF₃, Cl, Br, or OCH₃. In some embodiments, R^{c1} is phenyl or m-tolyl. In some embodiments, R^{c1} is pyridyl. In some embodiments, R^{c1} is 4-pyridyl.

In some embodiments, R^{4a} is phenyl or pyridyl, each optionally substituted with methyl or —CF₃. In some embodiments, R^{4a} is phenyl. In some embodiments, R^{4a} is tolyl. In some embodiments, R^{4a} is m-tolyl. In some embodiments, R^{4a} is pyridyl. In some embodiments, R^{4a} is 4-pyridyl.

In some embodiments, R^{4a} is pyrazolyl optionally substituted with one or two R^{z} groups.

In some embodiments, each R^{z} is independently methyl, ethyl, isopropyl, —CF₃, fluoro, chloro, —OCH₃, —OCF₃, methylamino, ethylamino, propylamino, butylamino, aminomethyl, aminoethyl, aminopropyl, aminobutyl, dimethylamino, dimethylaminomethyl, dimethylaminoethyl, dimethylaminopropyl, dimethylaminobutyl, —C(O)methylamino, —C(O)ethylamino, —C(O)propylamino, —C(O)butylamino, —C(O)dimethylamino, —C(O)dimethylaminomethyl, —C(O)dimethylaminoethyl, —C(O)dimethylaminopropyl, or —C(O)dimethylaminobutyl.

In some embodiments, R^{4a} is 3-methyl-1H-pyrazol-5-yl, 3-methylisothiazol-5-yl, 2-methyl-1H-imidazol-5-yl, 1-methyl-pyrazol-4-yl, 1-methylpyrazol-3-yl, 1-((1-acetamido)-eth-2-yl)-5-methyl-pyrazol-3-yl, 1-((1-chloromethylamido)-eth-2-yl)-5-methyl-pyrazol-3-yl, 1-((1-acrylamido)-eth-2-yl)-5-methyl-pyrazol-3-yl, thiazol-2-yl, pyrazol-4-yl, pyrazol-1-yl, oxazol-2-yl, or 3-(1-N,N-dimethyl-eth-2-yl)-4-methyl-pyrazol-1-yl.

In some embodiments, R^{4a} is —C(O)NR^{x}R^{y} wherein R^{x} is H or C₁₋₄alkyl and R^{y} is H, C₁₋₄alkyl, —O—C₁₋₄alkyl, —SO₂—R^{r}, C₁₋₄alkyl-SO₂—R^{r} monocyclic cycloalkyl, —C₁₋₄alkyl(monocyclic cycloalkyl), monocyclic heterocyclyl, or monocyclic heterocycloalkyl, each optionally substituted with one, two, or three R^{o} substituents; and R^{r} and R^{o} are as defined herein. In some embodiments, R^{4a} is —C(O)NR^{x}R^{y} wherein R^{x} is H or methyl; and R^{y} is H, methyl, ethyl, butyl, isopropyl, methoxy, —SO₂-methyl, C₂₋₄alkyl-SO₂-methyl, C₂₋₄alkyl-SO₂—N(CH₃)₂, cyclopropyl, cyclobutyl, cyclopentyl, cyclopropylmethyl, 1-cyclopropylethyl, 2-cyclopropylethyl, cyclobutylmethyl, cyclopentylmethyl, azetidinyl, oxetanyl, pyrrolidinyl, piperidinyl, morpholinyl, piperazinyl, azepanyl, azocanyl, tetrahydrofuranyl, tetrahydropyranyl, substituted azetidinylmethyl, oxetanylmethyl, pyrrolidinylmethyl, piperidinylmethyl, morpholinylmethyl, or piperazinylmethyl, each optionally substituted with one, two, or three methyl, methoxy, fluoro or amino groups.

In some embodiments, the compound of Formula (I) or the pharmaceutically acceptable salt thereof is a compound of

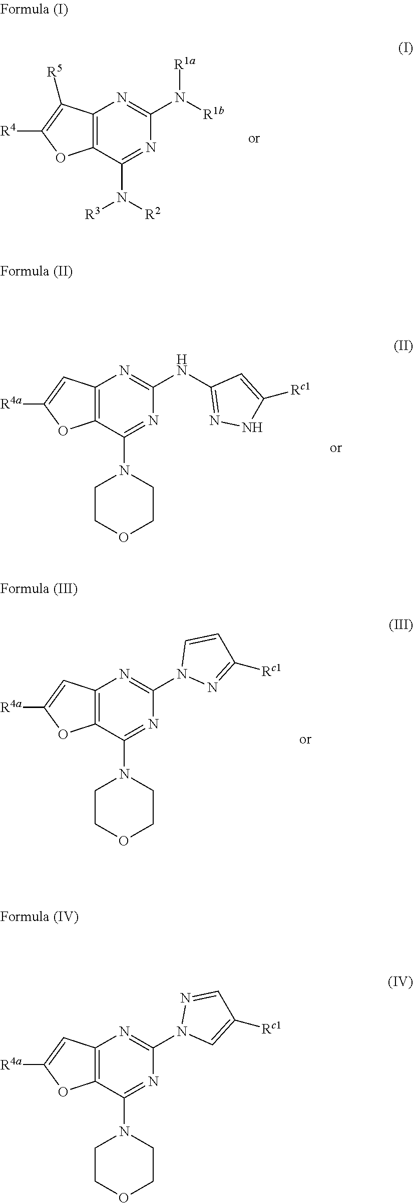

as defined herein, wherein one or more hydrogen atoms attached to carbon atoms of the compound are replaced by deuterium atoms.

In some embodiments, one or more hydrogen atoms attached to carbon atoms of R¹, R², R³, R⁴, R⁵, R^{1a}, R^{1b}, R^{1c} or R^{4a} are replaced by deuterium atoms.

In some embodiments, one or more hydrogen atoms attached to carbon atoms of R^{a}, R^{b}, R^{c}, R^{d}, R^{g}, R^{h}, R^{j}, R^{k}, R^{l}, R^{m}, Rⁿ, R^{o}, R^{p}, R^{q}, R^{r}, R^{x}, R^{y}, or R^{z} are replaced by deuterium atoms. In some embodiments, one or more R^{a}, R^{b}, R^{c}, R^{d}, R^{g}, R^{h}, R^{j}, R^{k}, R^{l}, R^{m}, Rⁿ, R^{o}, R^{p}, R^{q}, R^{r}, R^{x}, R^{y}, or R^{z} group is a C₁₋₄alkyl group wherein one or more hydrogen atoms attached to carbon atoms are replaced by deuterium atoms. In some embodiments, one or more R^{a}, R^{b}, R^{c}, R^{d}, R^{g}, R^{h}, R^{j}, R^{k}, R^{l}, R^{m}, Rⁿ, R^{o}, R^{p}, R^{q}, R^{r}, R^{x}, R^{y}, or R^{z} group is a methyl group wherein one or more hydrogen atoms attached to the carbon atom are replaced by deuterium atoms. In some embodiments, one or more R^{a}, R^{b}, R^{c}, R^{d}, R^{g}, R^{h}, R^{j}, R^{k}, R^{l}, R^{m}, Rⁿ, R^{o}, R^{p}, R^{q}, R^{r}, R^{x}, R^{y}, or R^{z} group is —CD₃.

In some embodiments, the compound of Formula (I)-(IV) comprises a -D in place of at least one —H, or a —CD₃ substituent in place of at least one CH₃.

In some embodiments, the compound is a compound selected from those of Table 1:

TABLE1

| Compound | | |
|---|---|---|
| # | Name | Structure |
| 1 | 4-morpholino-N-(5-phenyl-1H-pyrazol- 3-yl)-6-(4-pyridyl)furo[3,2-d]pyrimidin- 2-amine hydrogen chloride | 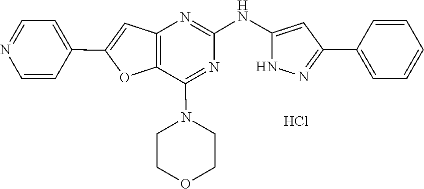 [SMILES: c1ccc(-c2cc(Nc3nc(N4CCOCC4)c4oc(-c5ccncc5)cc4n3)[nH]n2)cc1 ; Cl] |
| 2 | 4-morpholino-N-[5-(o-tolyl)-1H- pyrazol-3-yl]-6-(4-pyridyl)furo[3,2- d]pyrimidin-2-amine, hydrogen chloride | 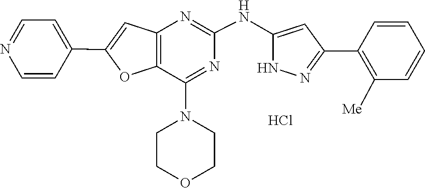 [SMILES: Cc1ccccc1-c1cc(Nc2nc(N3CCOCC3)c3oc(-c4ccncc4)cc3n2)[nH]n1 ; Cl] |
| 3 | 4-morpholino-N-[5-(m-tolyl)-1H- pyrazol-3-yl]-6-(4-pyridyl)furo[3,2- d]pyrimidin-2-amine, hydrogen chloride | 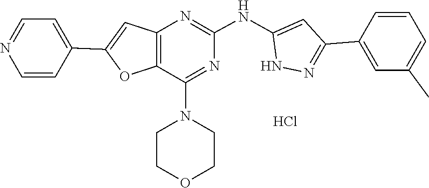 [SMILES: Cc1cccc(-c2cc(Nc3nc(N4CCOCC4)c4oc(-c5ccncc5)cc4n3)[nH]n2)c1 ; Cl] |
| 4 | 4-morpholino-N-[5-(p-tolyl)-1H- pyrazol-3-yl]-6-(4-pyridyl)furo[3,2- d]pyrimidin-2-amine, hydrogen chloride | 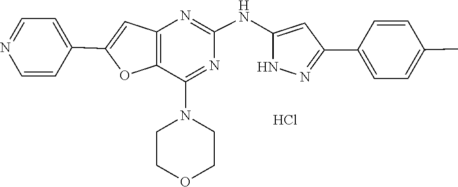 [SMILES: Cc1ccc(-c2cc(Nc3nc(N4CCOCC4)c4oc(-c5ccncc5)cc4n3)[nH]n2)cc1 ; Cl] |
| 5 | N-(5-methyl-1H-pyrazol-3-yl)-4- morpholino-6-(4-pyridyl)furo[3,2- d]pyrimidin-2-amine, hydrogen chloride | 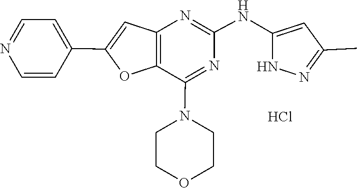 [SMILES: Cc1cc(Nc2nc(N3CCOCC3)c3oc(-c4ccncc4)cc3n2)[nH]n1 ; Cl] |
| 6 | 4-morpholino-6-(4-pyridyl)-N-[5-(4- pyridyl)-1H-pyrazol-3-yl]furo[3,2- d]pyrimidin-2-amine, hydrogen chloride | 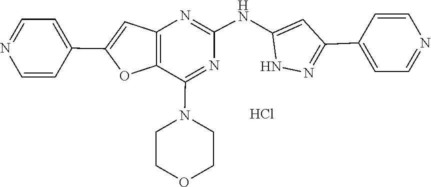 [SMILES: c1cc(-c2cc(Nc3nc(N4CCOCC4)c4oc(-c5ccncc5)cc4n3)[nH]n2)ccn1 ; Cl] |
| 7 | N-[5-(3-fluorophenyl)-1H-pyrazol-3-yl]- 4-morpholino-furo[3,2-d]pyrimidin-2- amine, hydrogen chloride | 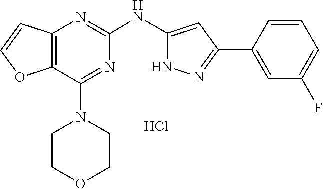 [SMILES: Fc1cccc(-c2cc(Nc3nc(N4CCOCC4)c4occc4n3)[nH]n2)c1 ; Cl] |
| 8 | 4-morpholino-N-[5-(m-tolyl)-1H- pyrazol-3-yl]furo[3,2-d]pyrimidin-2- amine, hydrogen chloride | 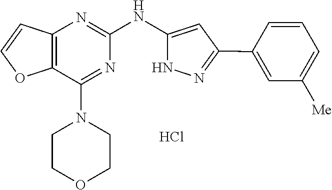 [SMILES: Cc1cccc(-c2cc(Nc3nc(N4CCOCC4)c4occc4n3)[nH]n2)c1 ; Cl] |
| 9 | N-(5-methyl-1H-pyrazol-3-yl)-4- morpholino-furo[3,2-d]pyrimidin-2- amine, hydrogen chloride | 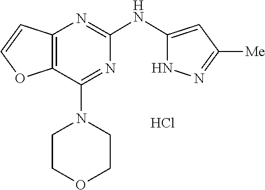 [SMILES: Cc1cc(Nc2nc(N3CCOCC3)c3occc3n2)[nH]n1 ; Cl] |
| 10 | 4-morpholino-N-[5-(4-pyridyl)-1H- pyrazol-3-yl]furo[3,2-d]pyrimidin-2- amine, hydrogen chloride | 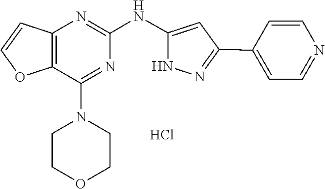 [SMILES: c1cc(-c2cc(Nc3nc(N4CCOCC4)c4occc4n3)[nH]n2)ccn1 ; Cl] |
| 11 | 6-chloro-4-morpholino-N-(5-phenyl-1H- pyrazol-3-yl)furo[3,2-d]pyrimidin-2- amine, hydrogen chloride | 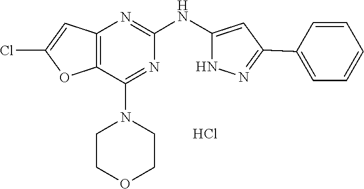 [SMILES: Clc1cc2nc(Nc3cc(-c4ccccc4)n[nH]3)nc(N3CCOCC3)c2o1 ; Cl] |
| 12 | 4-morpholino-6-(morpholinomethyl)-2- ((3-phenyl-1H-pyrazol-5- yl)methyl)furo[3,2-d]pyrimidine, hydrogen chloride | 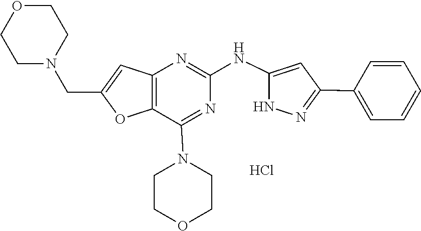 [SMILES: c1ccc(-c2cc(Nc3nc(N4CCOCC4)c4oc(CN5CCOCC5)cc4n3)[nH]n2)cc1 ; Cl] |
| 13 | 4-morpholino-2-((3-phenyl-1H-pyrazol- 5-yl)methyl)-6-(piperidin-1- ylmethyl)furo[3,2-d]pyrimidrne, hydrogen chloride | 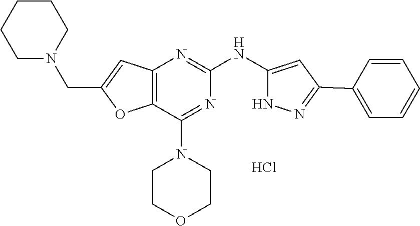 [SMILES: c1ccc(-c2cc(Nc3nc(N4CCOCC4)c4oc(CN5CCCCC5)cc4n3)[nH]n2)cc1 ; Cl] |
| 14 | N,N-dimethyl-1-(4-morpholino-2-((3- phenyl-1H-pyrazol-5- yl)methyl)furo[3,2-d]pyrimidin-6- yl)methanamine, hydrogen chloride | 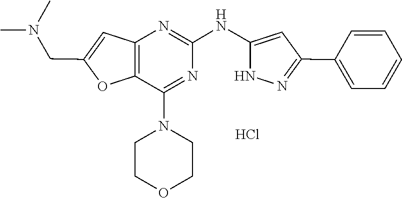 [SMILES: CN(C)Cc1cc2nc(Nc3cc(-c4ccccc4)n[nH]3)nc(N3CCOCC3)c2o1 ; Cl] |
| 15 | 6-((4-methylpiperazin-1-yl)methyl)-4- morpholino-2-((3-phenyl-1H-pyrazol-5- yl)methyl)furo[3,2-d]pyrimidine, hydrogen chloride | 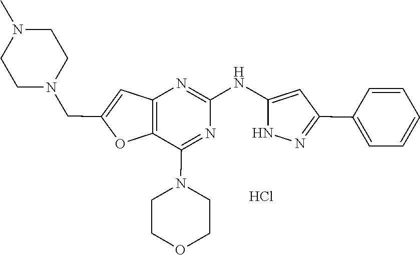 [SMILES: CN1CCN(Cc2cc3nc(Nc4cc(-c5ccccc5)n[nH]4)nc(N4CCOCC4)c3o2)CC1 ; Cl] |
| 16 | 4-morpholino-2-((3-phenyl-1H-pyrazol- 5-yl)methyl)-6-(pyrrolidin-1- ylmethyl)furo[3,2-d]pyrimidine, hydrogen chloride | 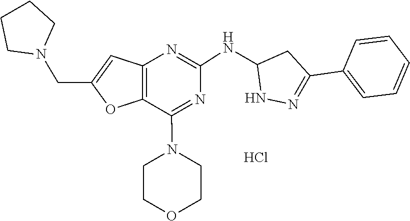 [SMILES: c1ccc(C2=NNC(Nc3nc(N4CCOCC4)c4oc(CN5CCCC5)cc4n3)C2)cc1 ; Cl] |
| 17 | 2-methoxy-N-methyl-N-((4-morpholino- 2-((3-phenyl-1H-pyrazol-5- yl)methyl)furo[3,2-d]pyrimidin-6- yl)methyl)ethan-1-amine hydrogen chloride | 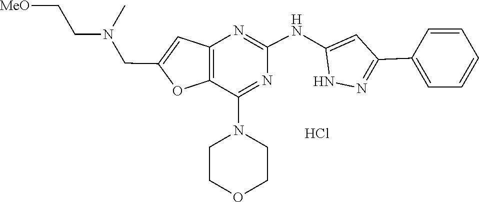 [SMILES: COCCN(C)Cc1cc2nc(Nc3cc(-c4ccccc4)n[nH]3)nc(N3CCOCC3)c2o1 ; Cl] |
| 18 | 4-morpholino-6-(4-pyridyl)-N-[5-(3- pyridyl)-1H-pyrazol-3-yl]furo[3,2- d]pyrimidin-2-amine, hydrogen chloride | 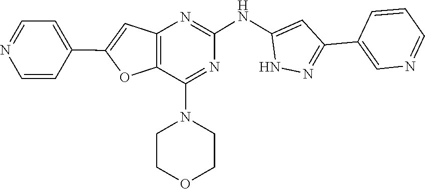 [SMILES: c1cncc(-c2cc(Nc3nc(N4CCOCC4)c4oc(-c5ccncc5)cc4n3)[nH]n2)c1] |
| 19 | 4-morpholino-6-(4-pyridyl)-N-[5-(2- pyridyl)-1H-pyrazol-3-yl]furo[3,2- d]pyrimidin-2-amine, hydrogen chloride | 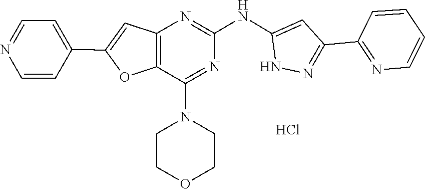 [SMILES: c1ccc(-c2cc(Nc3nc(N4CCOCC4)c4oc(-c5ccncc5)cc4n3)[nH]n2)nc1 ; Cl] |
| 20 | N-[5-(6-methyl-2-pyridyl)-1H-pyrazol- 3-yl]-4-morpholino-6-(4- pyridyl)furo[3,2-d]pyrimidin-2-amine, hydrogen chloride | 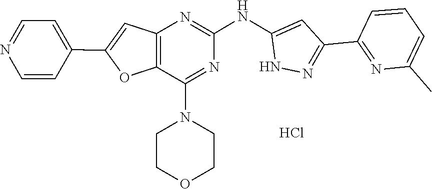 [SMILES: Cc1cccc(-c2cc(Nc3nc(N4CCOCC4)c4oc(-c5ccncc5)cc4n3)[nH]n2)n1 ; Cl] |
| 21 | N-[5-(4-methyl-2-pyridyl)-1H-pyrazol- 3-yl]-4-morpholino-6-(4- pyridyl)furo[3,2-d]pyrimidin-2-amine, hydrogen chloride | 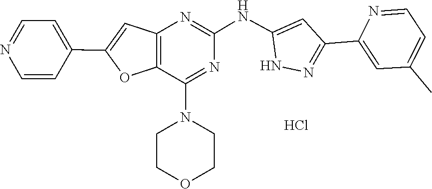 [SMILES: Cc1ccnc(-c2cc(Nc3nc(N4CCOCC4)c4oc(-c5ccncc5)cc4n3)[nH]n2)c1 ; Cl] |
| 22 | N-[5-(2-methyl-4-pyridyl)-1H-pyrazol- 3-yl]-4-morpholino-6-(4- pyridyl)furo[3,2-d]pyrimidin-2-amine, hydrogen chloride | 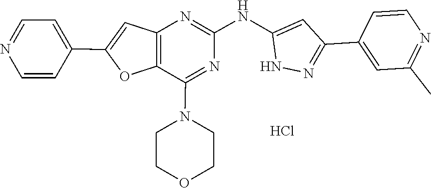 [SMILES: Cc1cc(-c2cc(Nc3nc(N4CCOCC4)c4oc(-c5ccncc5)cc4n3)[nH]n2)ccn1 ; Cl] |
| 23 | N-[5-(5-methyl-3-pyridyl)-1H-pyrazol- 3-yl]-4-morpholino-6-(4- pyridyl)furo[3,2-d]pyrimidin-2-amine, hydrogen chloride | 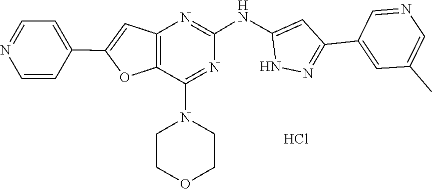 [SMILES: Cc1cncc(-c2cc(Nc3nc(N4CCOCC4)c4oc(-c5ccncc5)cc4n3)[nH]n2)c1 ; Cl] |
| 24 | N-[5-(3-methoxyphenyl)-1H-pyrazol-3- yl]-4-morpholino-6-(4-pyridyl)furo[3,2- d]pyrimidin-2-amine, hydrogen chloride | 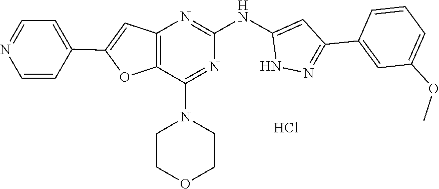 [SMILES: COc1cccc(-c2cc(Nc3nc(N4CCOCC4)c4oc(-c5ccncc5)cc4n3)[nH]n2)c1 ; Cl] |
| 25 | 4-morpholino-6-(4-pyridyl)-N-[5-[3- (trifluoromethoxy)phenyl]-1H-pyrazol- 3-yl]furo[3,2-d]pyrimidin-2-amine, hydrogen chloride | 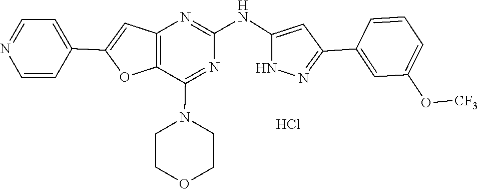 [SMILES: FC(F)(F)Oc1cccc(-c2cc(Nc3nc(N4CCOCC4)c4oc(-c5ccncc5)cc4n3)[nH]n2)c1 ; Cl] |
| 26 | 4-morpholino-N-(5-phenyl-1H-pyrazol- 3-yl)-6-(2-pyridyl)furo[3,2-d]pyrimidin- 2-amine, hydrogen chloride | 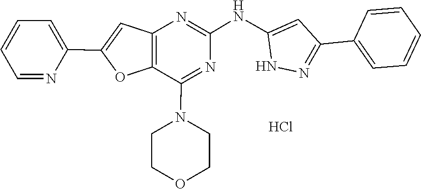 [SMILES: c1ccc(-c2cc(Nc3nc(N4CCOCC4)c4oc(-c5ccccn5)cc4n3)[nH]n2)cc1 ; Cl] |
| 27 | 4-morpholino-N-[5-(m-tolyl)-1H- pyrazol-3-yl]-6-(2-pyridyl)furo[3,2- d]pyrimidin-2-amine, hydrogen chloride | 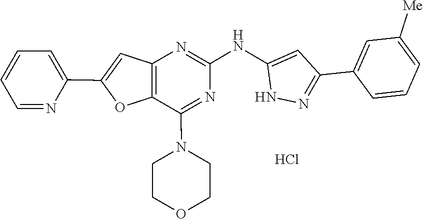 [SMILES: Cc1cccc(-c2cc(Nc3nc(N4CCOCC4)c4oc(-c5ccccn5)cc4n3)[nH]n2)c1 ; Cl] |
| 28 | 4-morpholino-6-(2-pyridyl)-N-[5-(4- pyridyl)-1H-pyrazol-3-yl]furo[3,2- d]pyrimidin-2-amine, hydrogen chloride | 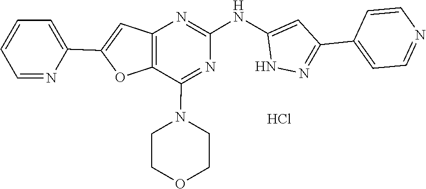 [SMILES: c1ccc(-c2cc3nc(Nc4cc(-c5ccncc5)n[nH]4)nc(N4CCOCC4)c3o2)nc1 ; Cl] |
| 29 | 4-morpholino-N-(5-phenyl-1H-pyrazol- 3-yl)-6-(3-pyridyl)furo[3,2-d]pyrimidin- 2-amine, hydrogen chloride | 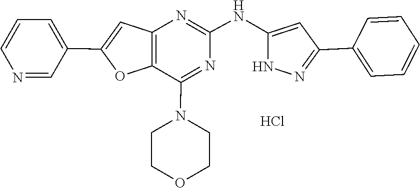 [SMILES: c1ccc(-c2cc(Nc3nc(N4CCOCC4)c4oc(-c5cccnc5)cc4n3)[nH]n2)cc1 ; Cl] |
| 30 | 4-morpholino-N-[5-(m-tolyl)-1H- pyrazol-3-yl]-6-(3-pyridyl)furo[3,2- d]pyrimidin-2-amine, hydrogen chloride | 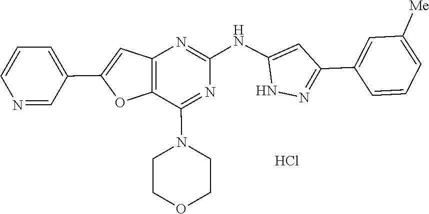 [SMILES: Cc1cccc(-c2cc(Nc3nc(N4CCOCC4)c4oc(-c5cccnc5)cc4n3)[nH]n2)c1 ; Cl] |
| 31 | 4-morpholino-6-(3-pyridyl)-N-[5-(4- pyridyl)-1H-pyrazol-3-yl]furo[3,2- d]pyrimidin-2-amine, hydrogen chloride | 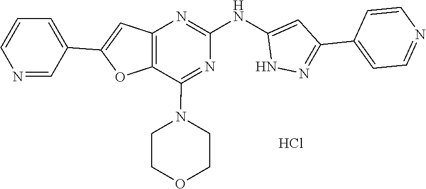 [SMILES: c1cncc(-c2cc3nc(Nc4cc(-c5ccncc5)n[nH]4)nc(N4CCOCC4)c3o2)c1 ; Cl] |
| 32 | 6-(1-methylpiperidin-4-yl)-4- morpholino-2-((3-phenyl-1H-pyrazol-5- yl)methyl)furo[3,2-d]pyrimidine, hydrogen chloride | 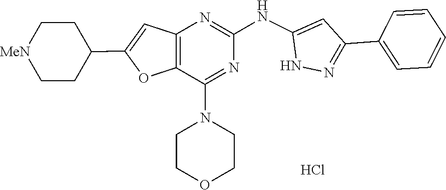 [SMILES: CN1CCC(c2cc3nc(Nc4cc(-c5ccccc5)n[nH]4)nc(N4CCOCC4)c3o2)CC1 ; Cl] |
| 33 | 6-(1-methylpiperidin-4-yl)-4- morpholino-2-((3-(m-tolyl)-1H-pyrazol- 5-yl)methyl)furo[3,2-d]pyrimidine, hydrogen chloride | 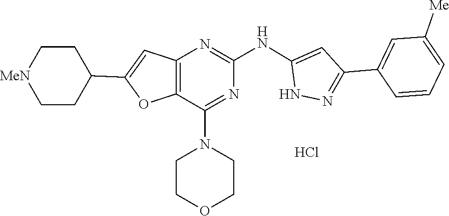 [SMILES: Cc1cccc(-c2cc(Nc3nc(N4CCOCC4)c4oc(C5CCN(C)CC5)cc4n3)[nH]n2)c1 ; Cl] |
| 34 | 6-(1-methylpiperidin-4-yl)-4- morpholino-2-((3-(pyridin-4-yl)-1H- pyrazol-5-yl)methyl)furo[3,2- d]pyrimidine, hydrogen chloride | 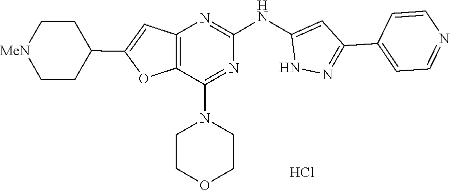 [SMILES: CN1CCC(c2cc3nc(Nc4cc(-c5ccncc5)n[nH]4)nc(N4CCOCC4)c3o2)CC1 ; Cl] |
| 35 | 6-(1-methylpiperidin-3-yl)-4- morpholino-2-((3-phenyl-1H-pyrazol-5- yl)methyl)furo[3,2-d]pyrimidine, hydrogen chloride | 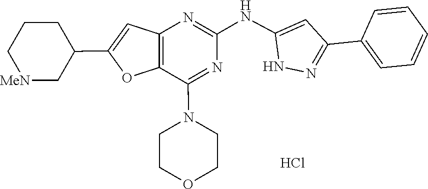 [SMILES: CN1CCCC(c2cc3nc(Nc4cc(-c5ccccc5)n[nH]4)nc(N4CCOCC4)c3o2)C1 ; Cl] |
| 36 | 6-(1-methylpiperidin-3-yl)-4- morpholino-2-((3-(m-tolyl)-1H-pyrazol- 5-yl)methyl)furo[3,2-d]pyrimidine, hydrogen chloride | 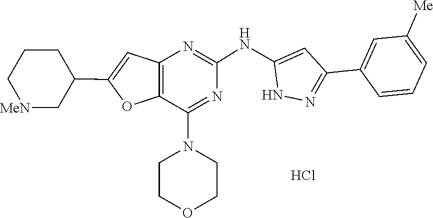 [SMILES: Cc1cccc(-c2cc(Nc3nc(N4CCOCC4)c4oc(C5CCCN(C)C5)cc4n3)[nH]n2)c1 ; Cl] |
| 37 | 6-(1-methylpiperidin-3-yl)-4- morpholino-2-((3-(pyridin-4-yl)-1H- pyrazol-5-yl)methyl)furo[3,2- d]pyrimidine, hydrogen chloride | 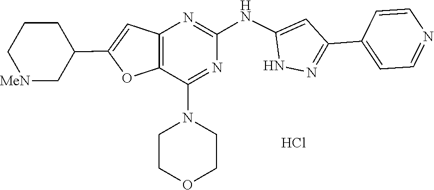 [SMILES: CN1CCCC(c2cc3nc(Nc4cc(-c5ccncc5)n[nH]4)nc(N4CCOCC4)c3o2)C1 ; Cl] |
| 38 | 6-(1-methylpyrrolidin-3-yl)-4- morpholino-2-((3-phenyl-1H-pyrazol-5- yl)methyl)furo[3,2-d]pyrimidine, hydrogen chloride | 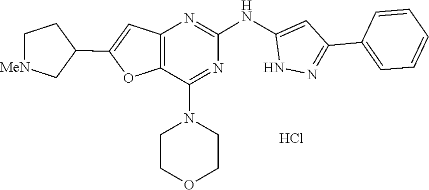 [SMILES: CN1CCC(c2cc3nc(Nc4cc(-c5ccccc5)n[nH]4)nc(N4CCOCC4)c3o2)C1 ; Cl] |
| 39 | 6-(1-methylpyrrolidin-3-yl)-4- morpholino-2-((3-(m-tolyl)-1H-pyrazol- 5-yl)methyl)furo[3,2-d]pyrimidine, hydrogen chloride | 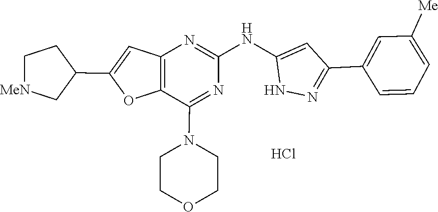 [SMILES: Cc1cccc(-c2cc(Nc3nc(N4CCOCC4)c4oc(C5CCN(C)C5)cc4n3)[nH]n2)c1 ; Cl] |
| 40 | 6-(1-methylpyrrolidin-3-yl)-4- morpholino-2-((3-(pyridin-4-yl)-1H- pyrazol-5-yl)methyl)furo[3,2- d]pyrimidine, hydrogen chloride | 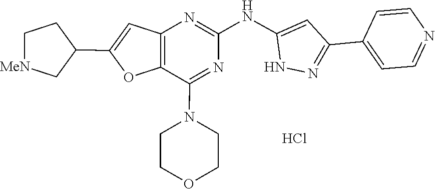 [SMILES: CN1CCC(c2cc3nc(Nc4cc(-c5ccncc5)n[nH]4)nc(N4CCOCC4)c3o2)C1 ; Cl] |
| 41 | 4-morpholino-2-(4-phenylpyrazol-1-yl)- 6-(4-pyridyl)furo[3,2-d]pyrimidine, hydrogen chloride | 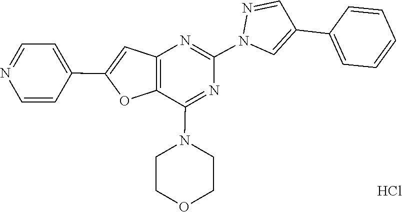 [SMILES: c1ccc(-c2cnn(-c3nc(N4CCOCC4)c4oc(-c5ccncc5)cc4n3)c2)cc1 ; Cl] |
| 42 | 4-morpholino-2-[4-(m-tolyl)pyrazol-1- yl]-6-(4-pyridyl)furo[3,2-d]pyrimidine, hydrogen chloride | 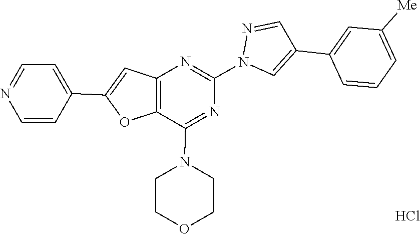 [SMILES: Cc1cccc(-c2cnn(-c3nc(N4CCOCC4)c4oc(-c5ccncc5)cc4n3)c2)c1 ; Cl] |
| 43 | 4-morpholino-N-(5-phenylisoxazol-3- yl)-6-(4-pyridyl)furo[3,2-d]pyrimidin-2- amine, hydrogen chloride | 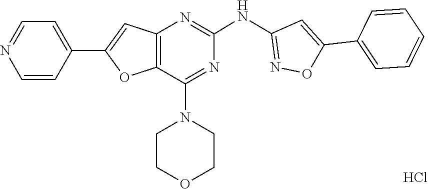 [SMILES: c1ccc(-c2cc(Nc3nc(N4CCOCC4)c4oc(-c5ccncc5)cc4n3)no2)cc1 ; Cl] |
| 44 | 4-morpholino-N-(3-phenylisoxazol-5- yl)-6-(4-pyridyl)furo[3,2-d]pyrimidin-2- amine, hydrogen chloride | 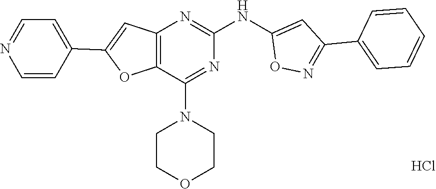 [SMILES: c1ccc(-c2cc(Nc3nc(N4CCOCC4)c4oc(-c5ccncc5)cc4n3)on2)cc1 ; Cl] |
| 45 | 4-morpholino-N-(4-phenyloxazol-2-yl)- 6-(4-pyridyl)furo[3,2-d]pyrimidin-2- amine, hydrogen chloride | 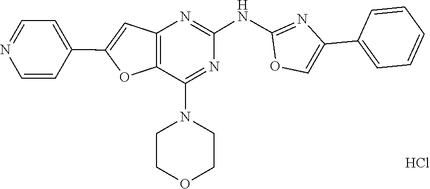 [SMILES: c1ccc(-c2coc(Nc3nc(N4CCOCC4)c4oc(-c5ccncc5)cc4n3)n2)cc1 ; Cl] |
| 46 | 4-morpholino-N-(5-phenyloxazol-2-yl)- 6-(4-pyridyl)furo[3,2-d]pyrimidin-2- amine, hydrogen chloride | 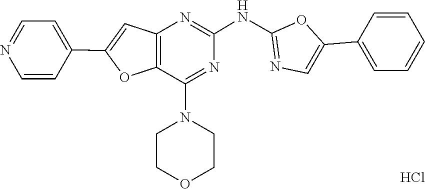 [SMILES: c1ccc(-c2cnc(Nc3nc(N4CCOCC4)c4oc(-c5ccncc5)cc4n3)o2)cc1 ; Cl] |
| 47 | 4-morpholino-N-(5-phenyl-1,3,4- oxadiazol-2-yl)-6-(4-pyridyl)furo[3,2- d]pyrimidin-2-amine, hydrogen chloride | 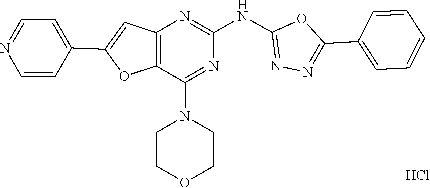 [SMILES: c1ccc(-c2nnc(Nc3nc(N4CCOCC4)c4oc(-c5ccncc5)cc4n3)o2)cc1 ; Cl] |
| 48 | 4-morpholino-N-(1-phenylpyrazol-3-yl)- 6-(4-pyridyl)furo[3,2-d]pyrimidin-2- amine, hydrogen chloride | 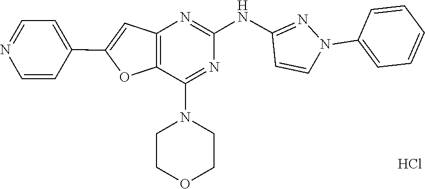 [SMILES: c1ccc(-n2ccc(Nc3nc(N4CCOCC4)c4oc(-c5ccncc5)cc4n3)n2)cc1 ; Cl] |
| 49 | 4-morpholino-N-(1-phenylpyrazol-4-yl)- 6-(4-pyridyl)furo[3,2-d]pyrimidin-2- amine, hydrogen chloride | 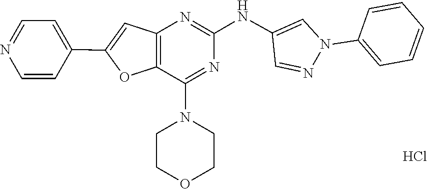 [SMILES: c1ccc(-n2cc(Nc3nc(N4CCOCC4)c4oc(-c5ccncc5)cc4n3)cn2)cc1 ; Cl] |
| 50 | N-(1H-indazol-3-yl)-4-morpholino-6-(4- pyridyl)furo[3,2-d]pyrimidin-2-amine, hydrogen chloride | 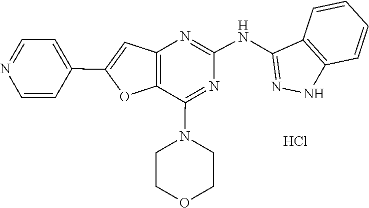 [SMILES: c1ccc2c(Nc3nc(N4CCOCC4)c4oc(-c5ccncc5)cc4n3)n[nH]c2c1 ; Cl] |
| 51 | 4-morpholino-N-pyrazolo[1,5-a]pyridin- 2-yl-6-(4-pyridyl)furo[3,2-d]pyrimidin- 2-amine, hydrogen chloride | 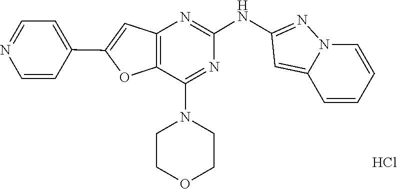 [SMILES: c1ccn2nc(Nc3nc(N4CCOCC4)c4oc(-c5ccncc5)cc4n3)cc2c1 ; Cl] |
| 52 | 4-morpholino-2-[(5-phenyl-1H-pyrazol- 3-yl)amino]furo[3,2-d]pyrimidine-6- carboxamide, hydrogen chloride | 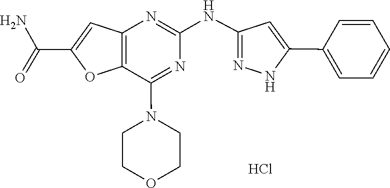 [SMILES: NC(=O)c1cc2nc(Nc3cc(-c4ccccc4)[nH]n3)nc(N3CCOCC3)c2o1 ; Cl] |
| 53 | azetidin-1-yl-[4-morpholino-2-[(5- phenyl-1H-pyrazol-3-yl)amino]furo[3,2- d]pyrimidin-6-yl]methanone, hydrogen chloride | 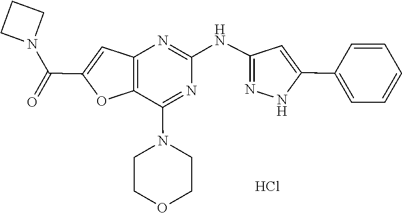 [SMILES: O=C(c1cc2nc(Nc3cc(-c4ccccc4)[nH]n3)nc(N3CCOCC3)c2o1)N1CCC1 ; Cl] |
| 54 | [4-morpholino-2-[(5-phenyl-1H- pyrazol-3-yl)amino]furo[3,2- d]pyrimidin-6-yl]-pyrrolidin-1-yl- methanone, hydrogen chloride | 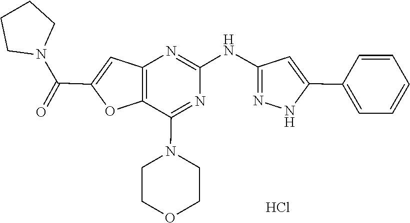 [SMILES: O=C(c1cc2nc(Nc3cc(-c4ccccc4)[nH]n3)nc(N3CCOCC3)c2o1)N1CCCC1 ; Cl] |
| 55 | [4-morpholino-2-[(5-phenyl-1H- pyrazol-3-yl)amino]furo[3,2- d]pyrimidin-6-yl]-(1- piperidyl) methanone, hydrogen chloride | 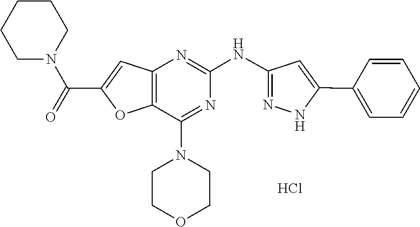 [SMILES: O=C(c1cc2nc(Nc3cc(-c4ccccc4)[nH]n3)nc(N3CCOCC3)c2o1)N1CCCCC1 ; Cl] |
| 56 | N-ethyl-4-morphohno-2-[(5-phenyl-1H- pyrazol-3-yl)amino]furo[3,2- d]pyrimidine-6-carboxamide, hydrogen chloride | 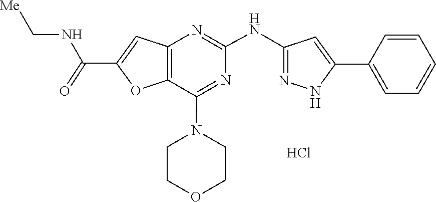 [SMILES: CCNC(=O)c1cc2nc(Nc3cc(-c4ccccc4)[nH]n3)nc(N3CCOCC3)c2o1 ; Cl] |
| 57 | N-ethyl-N-methyl-4-morpholino-2-[(5- phenyl-1H-pyrazol-3-yl)amino]furo[3,2- d]pyrimidine-6-carboxamide, hydrogen chloride | 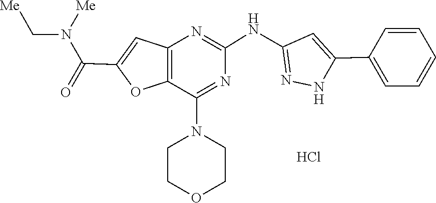 [SMILES: CCN(C)C(=O)c1cc2nc(Nc3cc(-c4ccccc4)[nH]n3)nc(N3CCOCC3)c2o1 ; Cl] |
| 58 | N-methoxy-4-morpholino-2-[(5-phenyl- 1H-pyrazol-3-yl)amino]furo[3,2- d]pyrimidine-6-carboxamide, hydrogen chloride | 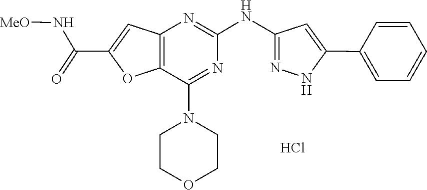 [SMILES: CONC(=O)c1cc2nc(Nc3cc(-c4ccccc4)[nH]n3)nc(N3CCOCC3)c2o1 ; Cl] |
| 59 | morpholino-2-[(5-phenyl-1H-pyrazol-3- yl)amino]furo[3,2-d]pyrimidine-6- carboxamide, hydrogen chloride | 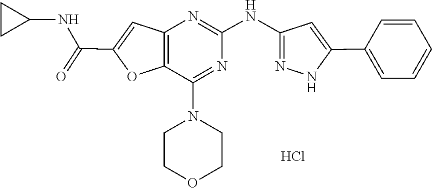 [SMILES: O=C(NC1CC1)c1cc2nc(Nc3cc(-c4ccccc4)[nH]n3)nc(N3CCOCC3)c2o1 ; Cl] |
| 60 | N-cyclopropyl-N-methyl-4-morpholino- 2-[(5-phenyl-1H-pyrazol-3- yl)amino]furo[3,2-d]pyrimidine-6- carboxamide, hydrogen chloride | 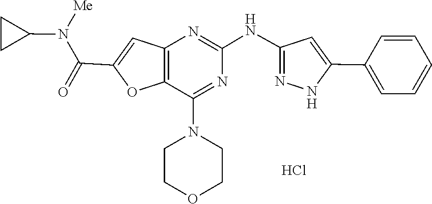 [SMILES: CN(C(=O)c1cc2nc(Nc3cc(-c4ccccc4)[nH]n3)nc(N3CCOCC3)c2o1)C1CC1 ; Cl] |
| 61 | N-(cyclopropylmethyl)-4-morpholino-2- [(5-phenyl-1H-pyrazol-3- yl)amino]furo[3,2-d]pyrimidine-6- carboxamide, hydrogen chloride | 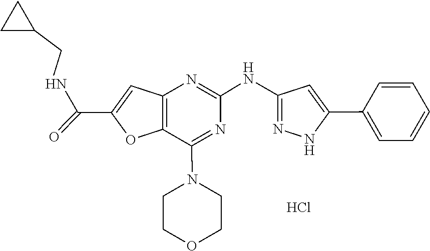 [SMILES: O=C(NCC1CC1)c1cc2nc(Nc3cc(-c4ccccc4)[nH]n3)nc(N3CCOCC3)c2o1 ; Cl] |
| 62 | N-(cyclopropylmethyl)-N-methyl-4- morpholino-2-[(5-phenyl-1H-pyrazol-3- yl)amino]furo[3,2-d]pyrimidine-6- carboxamide, hydrogen chloride | 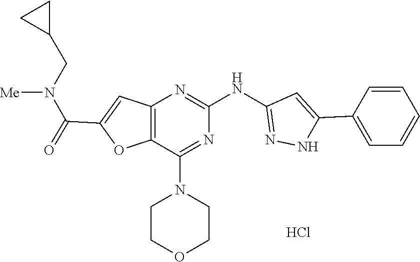 [SMILES: CN(CC1CC1)C(=O)c1cc2nc(Nc3cc(-c4ccccc4)[nH]n3)nc(N3CCOCC3)c2o1 ; Cl] |
| 63 | 4-morphohno-2-[(5-phenyl-1H-pyrazol- 3-yl)amino]-N-[(1S)-1- cyclopropylethyl]furo[3,2-d]pyrimidine- 6-carboxamide, hydrogen chloride | 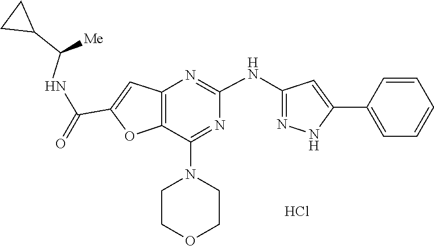 [SMILES: C[C@@H](NC(=O)c1cc2nc(Nc3cc(-c4ccccc4)[nH]n3)nc(N3CCOCC3)c2o1)C1CC1 ; Cl] |
| 64 | 4-morphohno-2-[(5-phenyl-1H-pyrazol- 3-yl)amino]-N-[(1R)-1- cyclopropylethyl]furo[3,2-d]pyrimidine- 6-carboxamide, hydrogen chloride | 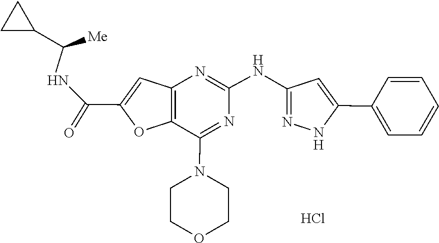 [SMILES: C[C@@H](NC(=O)c1cc2nc(Nc3cc(-c4ccccc4)[nH]n3)nc(N3CCOCC3)c2o1)C1CC1 ; Cl] |
| 65 | N-methylsulfonyl-4-morpholino-2-[(5- phenyl-1H-pyrazol-3-yl)amino]furo[3,2- d]pyrimidine-6-carboxamide, hydrogen chloride | 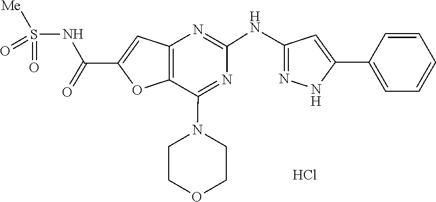 [SMILES: CS(=O)(=O)NC(=O)c1cc2nc(Nc3cc(-c4ccccc4)[nH]n3)nc(N3CCOCC3)c2o1 ; Cl] |
| 66 | N-(cyclopropylmethyl)-N-methyl-4- morpholino-2-(4-phenyl-1H-pyrazol-1- yl)furo[3,2-d]pyrimidine-6-carboxamide | 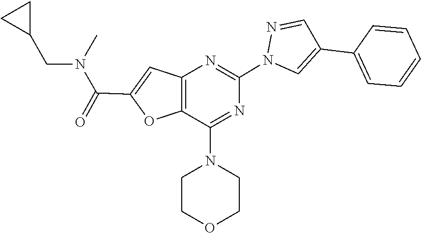 [SMILES: CN(CC1CC1)C(=O)c1cc2nc(-n3cc(-c4ccccc4)cn3)nc(N3CCOCC3)c2o1] |
| 67 | azetidin-1-yl(4-morpholino-2-(4-phenyl- 1H-pyrazol-1-yl)furo[3,2-d]pyrimidin-6- yl)methanone | 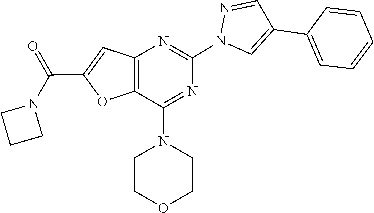 [SMILES: O=C(c1cc2nc(-n3cc(-c4ccccc4)cn3)nc(N3CCOCC3)c2o1)N1CCC1] |
| 68 | N-(5-cyclopropyl-1H-pyrazol-3-yl)-4- morpholino-6-(pyridin-4-yl)furo[3,2- d]pyrimidin-2-amine, hydrogen chloride | 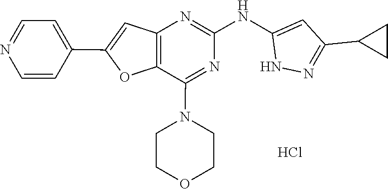 [SMILES: c1cc(-c2cc3nc(Nc4cc(C5CC5)n[nH]4)nc(N4CCOCC4)c3o2)ccn1 ; Cl] |
| 69 | N-(5-(cyclopropylmethyl)-1H-pyrazol- 3-yl)-4-morpholino-6-(pyridin-4- yl)furo[3,2-d]pyrimidin-2-amine, hydrogen chloride | 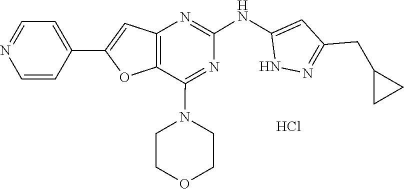 [SMILES: c1cc(-c2cc3nc(Nc4cc(CC5CC5)n[nH]4)nc(N4CCOCC4)c3o2)ccn1 ; Cl] |
| 70 | N-(5-cyclobutyl-1H-pyrazol-3-yl)-4- morpholino-6-(pyridin-4-yl)furo[3,2- d]pyrimidin-2-amine, hydrogen chloride | 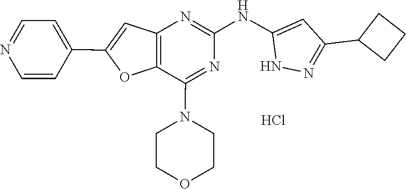 [SMILES: c1cc(-c2cc3nc(Nc4cc(C5CCC5)n[nH]4)nc(N4CCOCC4)c3o2)ccn1 ; Cl] |
| 71 | 2-((3-(cyclobutylmethyl)-1H-pyrazol-5- yl)methyl)-4-morpholino-6-(pyridin-4- yl)furo[3,2-d]pyrimidine, hydrogen chloride | 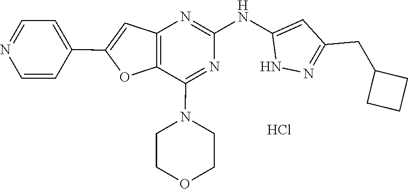 [SMILES: c1cc(-c2cc3nc(Nc4cc(CC5CCC5)n[nH]4)nc(N4CCOCC4)c3o2)ccn1 ; Cl] |
| 72 | 4-morpholino-6-(pyridin-4-yl)-N-(5- (trifluoromethyl)-1H-pyrazol-3- yl)furo[3,2-d]pyrimidin-2-amine, hydrogen chloride | 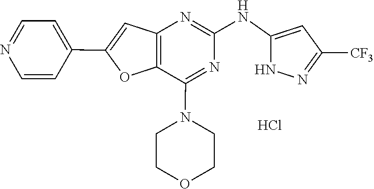 [SMILES: FC(F)(F)c1cc(Nc2nc(N3CCOCC3)c3oc(-c4ccncc4)cc3n2)[nH]n1 ; Cl] |
| 73 | N-(5-benzyl-1H-pyrazol-3-yl)-4- morpholino-6-(pyridin-4-yl)furo[3,2- d]pyrimidin-2-amine, hydrogen chloride | 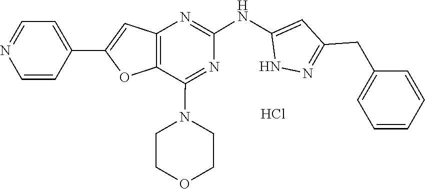 [SMILES: c1ccc(Cc2cc(Nc3nc(N4CCOCC4)c4oc(-c5ccncc5)cc4n3)[nH]n2)cc1 ; Cl] |
| 74 | N-(5-cyclopentyl-1H-pyrazol-3-yl)-4- morpholino-6-(pyridin-4-yl)furo[3,2- d]pyrimidin-2-amine, hydrogen chloride | 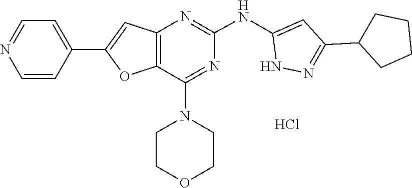 [SMILES: c1cc(-c2cc3nc(Nc4cc(C5CCCC5)n[nH]4)nc(N4CCOCC4)c3o2)ccn1 ; Cl] |
| 75 | 4-morpholino-6-(pyridin-4-yl)-N-(5- (thiophen-2-yl)-1H-pyrazol-3- yl)furo[3,2-d]pyrimidin-2-amine, hydrogen chloride | 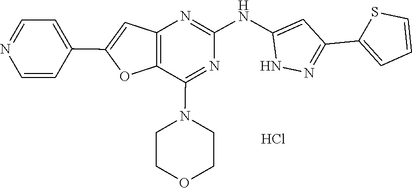 [SMILES: c1csc(-c2cc(Nc3nc(N4CCOCC4)c4oc(-c5ccncc5)cc4n3)[nH]n2)c1 ; Cl] |
| 76 | 4-morpholino-6-(pyridin-4-yl)-N-(5- (thiophen-3-yl)-1H-pyrazol-3- yl)furo[3,2-d]pyrimidin-2-amine, hydrogen chloride | 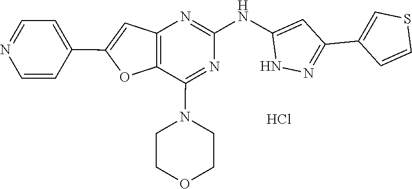 [SMILES: c1cc(-c2cc3nc(Nc4cc(-c5ccsc5)n[nH]4)nc(N4CCOCC4)c3o2)ccn1 ; Cl] |
| 77 | N-(5-isopropyl-1H-pyrazol-3-yl)-4- morpholino-6-(pyridin-4-yl)furo[3,2- d]pyrimidin-2-amine, hydrogen chloride | 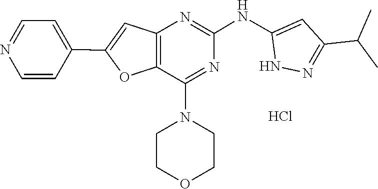 [SMILES: CC(C)c1cc(Nc2nc(N3CCOCC3)c3oc(-c4ccncc4)cc3n2)[nH]n1 ; Cl] |
| 78 | 2-((5-cyclopropyl-1H-pyrazol-3- yl)amino)-N-methyl-4- morpholinofuro[3,2-d]pyrimidine-6- carboxamide, hydrogen chloride | 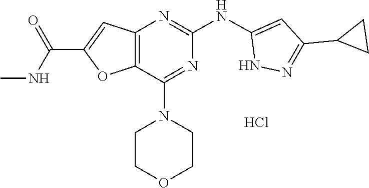 [SMILES: CNC(=O)c1cc2nc(Nc3cc(C4CC4)n[nH]3)nc(N3CCOCC3)c2o1 ; Cl] |
| 79 | 2-((5-(cyclopropylmethyl)-1H-pyrazol- 3-yl)amino)-N-methyl-4- morpholinofuro[3,2-d]pyrimidine-6- carboxamide, hydrogen chloride | 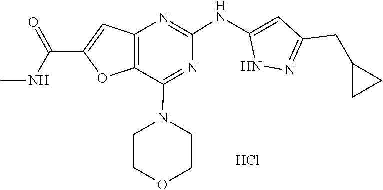 [SMILES: CNC(=O)c1cc2nc(Nc3cc(CC4CC4)n[nH]3)nc(N3CCOCC3)c2o1 ; Cl] |
| 80 | 2-((5-cyclobutyl-1H-pyrazol-3- yl)amino)-N-methyl-4- morpholinofuro[3,2-d]pyrimidine-6- carboxamide, hydrogen chloride | 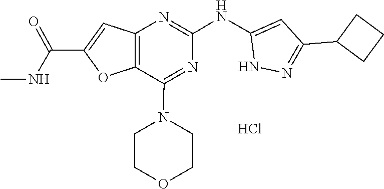 [SMILES: CNC(=O)c1cc2nc(Nc3cc(C4CCC4)n[nH]3)nc(N3CCOCC3)c2o1 ; Cl] |
| 81 | N-methyl-4-morpholino-2-((5- (trifluoromethyl)-1H-pyrazol-3- yl)amino)furo[3,2-d]pyrimidine-6- carboxamide, hydrogen chloride | 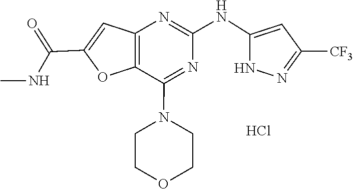 [SMILES: CNC(=O)c1cc2nc(Nc3cc(C(F)(F)F)n[nH]3)nc(N3CCOCC3)c2o1 ; Cl] |
| 82 | 4-morpholino-N-[5-(4-pyridyl)-1H- pyrazol-3-yl]furo[3,2-d]pyrimidin-2- amine, hydrogen chloride | 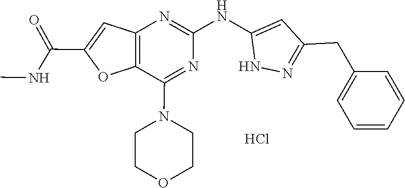 [SMILES: CNC(=O)c1cc2nc(Nc3cc(Cc4ccccc4)n[nH]3)nc(N3CCOCC3)c2o1 ; Cl] |
| 83 | 2-((5-cyclopentyl-1H-pyrazol-3- yl)amino)-N-methyl-4- morpholinofuro[3,2-d]pyrimidine-6- carboxamide, hydrogen chloride | 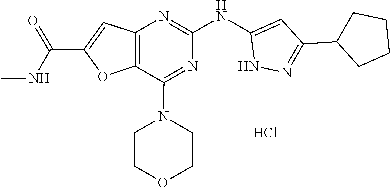 [SMILES: CNC(=O)c1cc2nc(Nc3cc(C4CCCC4)n[nH]3)nc(N3CCOCC3)c2o1 ; Cl] |
| 84 | 2-((5-(cyclopentylmethyl)-1H-pyrazol- 3-yl)amino)-N-methyl-4- morpholinofuro[3,2-d]pyrimidine-6- carboxamide, hydrogen chloride | 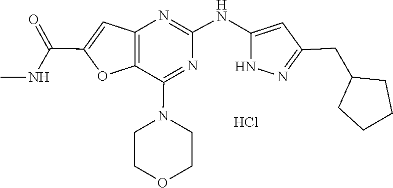 [SMILES: CNC(=O)c1cc2nc(Nc3cc(CC4CCCC4)n[nH]3)nc(N3CCOCC3)c2o1 ; Cl] |
| 85 | N-methyl-4-morpholino-2-((5- (thiophen-2-yl)-1H-pyrazol-3- yl)amino)furo[3,2-d]pyrimidine-6- carboxamide, hydrogen chloride | 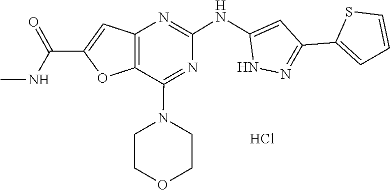 [SMILES: CNC(=O)c1cc2nc(Nc3cc(-c4cccs4)n[nH]3)nc(N3CCOCC3)c2o1 ; Cl] |
| 86 | N-methyl-4-morpholino-2-((5- (thiophen-3-yl)-1H-pyrazol-3- yl)amino)furo[3,2-d]pyrimidine-6- carboxamide, hydrogen chloride | 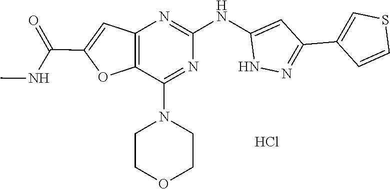 [SMILES: CNC(=O)c1cc2nc(Nc3cc(-c4ccsc4)n[nH]3)nc(N3CCOCC3)c2o1 ; Cl] |
| 87 | 2-((5-ethyl-1H-pyrazol-3-yl)amino)-N- methyl-4-morpholinofuro[3,2- d]pyrimidine-6-carboxamide, hydrogen chloride | 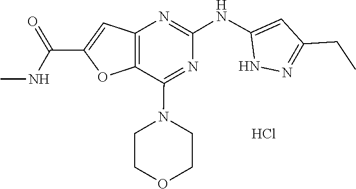 [SMILES: CCc1cc(Nc2nc(N3CCOCC3)c3oc(C(=O)NC)cc3n2)[nH]n1 ; Cl] |
| 88 | 2-((5-isopropyl-1H-pyrazol-3- yl)amino)-N-methyl-4- morpholinofuro[3,2-d]pyrimidine-6- carboxamide, hydrogen chloride | 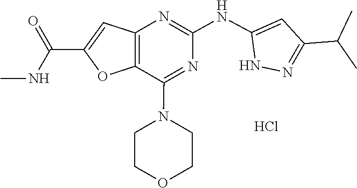 [SMILES: CNC(=O)c1cc2nc(Nc3cc(C(C)C)n[nH]3)nc(N3CCOCC3)c2o1 ; Cl] |
| 89 | 4-morpholino-6-(pyridin-4-yl)-2-(3-(m- tolyl)-1H-pyrazol-1-yl)furo[3,2- d]pyrimidine | 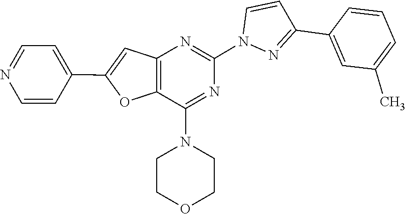 [SMILES: Cc1cccc(-c2ccn(-c3nc(N4CCOCC4)c4oc(-c5ccncc5)cc4n3)n2)c1] |
| 90 | 4-morpholino-6-(pyridin-3-yl)-2-(3-(m- tolyl)-1H-pyrazol-1-yl)furo[3,2- d]pyrimidine | 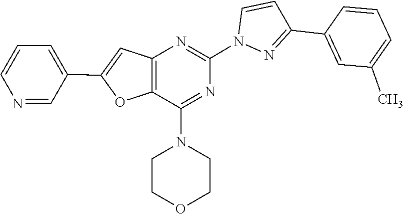 [SMILES: Cc1cccc(-c2ccn(-c3nc(N4CCOCC4)c4oc(-c5cccnc5)cc4n3)n2)c1] |
| 91 | 4-morpholino-6-(pyridin-2-yl)-2-(3-(m- tolyl)-1H-pyrazol-1-yl)furo[3,2- d]pyrimidine | 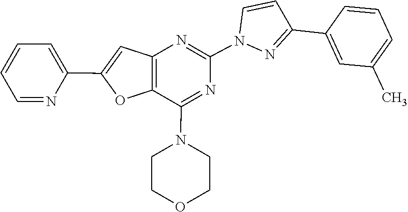 [SMILES: Cc1cccc(-c2ccn(-c3nc(N4CCOCC4)c4oc(-c5ccccn5)cc4n3)n2)c1] |
| 92 | N-methyl-4-morpholino-2-(3-(/w-tolyl)- 1H-pyrazol-1-yl)furo[3,2-d]pyrimidine- 6-carboxamide | 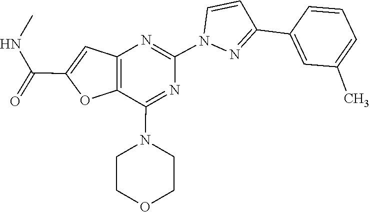 [SMILES: CNC(=O)c1cc2nc(-n3ccc(-c4cccc(C)c4)n3)nc(N3CCOCC3)c2o1] |
| 93 | H-(2-methoxyethyl)-4-morpholino-2-(3- (m-tolyl)-1H-pyrazol-1-yl)furo[3,2- d]pyrimidine-6-carboxamide | 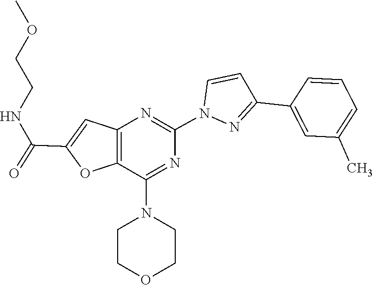 [SMILES: COCCNC(=O)c1cc2nc(-n3ccc(-c4cccc(C)c4)n3)nc(N3CCOCC3)c2o1] |
| 94 | 4-morpholino-2-(3-(m-tolyl)-1H- pyrazol-1-yl)furo[3,2-d]pyrimidine-6- carboxamide | 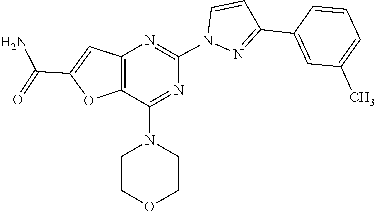 [SMILES: Cc1cccc(-c2ccn(-c3nc(N4CCOCC4)c4oc(C(N)=O)cc4n3)n2)c1] |
| 95 | N-ethyl-4-morpholino-2-(3-(m-tolyl)- 1H-pyrazol-1-yl)furo[3,2-d]pyrimidine- 6-carboxamide | 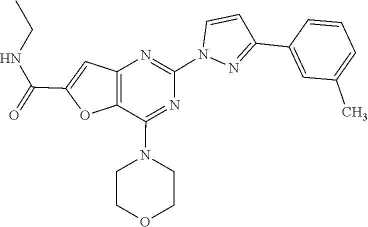 [SMILES: CCNC(=O)c1cc2nc(-n3ccc(-c4cccc(C)c4)n3)nc(N3CCOCC3)c2o1] |
| 96 | N-cyclopropyl-4-morpholino-2-(3-(m- tolyl)-1H-pyrazol-1-yl)furo[3,2- d]pyrimidine-6-carboxamide | 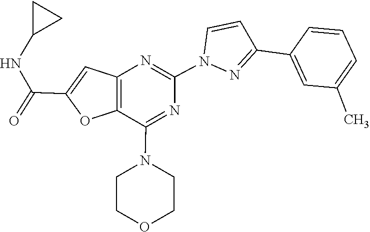 [SMILES: Cc1cccc(-c2ccn(-c3nc(N4CCOCC4)c4oc(C(=O)NC5CC5)cc4n3)n2)c1] |
| 97 | N-(cyclopropylmethyl)-4-morpholino-2- (3-(m-tolyl)-1H-pyrazol-1-yl)furo[3,2- d]pyrimidine-6-carboxamide | 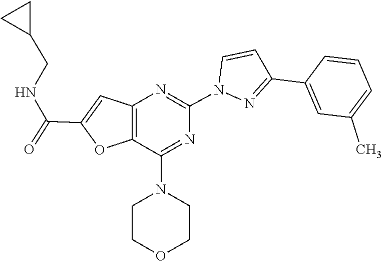 [SMILES: Cc1cccc(-c2ccn(-c3nc(N4CCOCC4)c4oc(C(=O)NCC5CC5)cc4n3)n2)c1] |
| 98 | N-(methylsulfonyl)-4-morpholino-2-(3- (m-tolyl)-1H-pyrazol-1-yl)furo[3,2- d]pyrimidine-6-carboxamide | 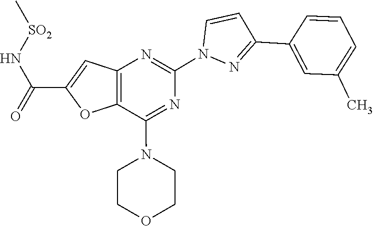 [SMILES: Cc1cccc(-c2ccn(-c3nc(N4CCOCC4)c4oc(C(=O)NS(C)(=O)=O)cc4n3)n2)c1] |
| 99 | N-methoxy-4-morpholino-2-(3-(m- tolyl)-1H-pyrazol-1-yl)furo[3,2- d]pyrimidine-6-carboxamide | 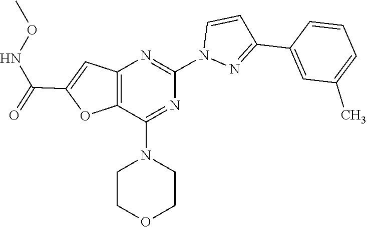 [SMILES: CONC(=O)c1cc2nc(-n3ccc(-c4cccc(C)c4)n3)nc(N3CCOCC3)c2o1] |
| 100 | N-cyclopropyl-4-morpholino-2-(4-(m- tolyl)-1H-pyrazol-1-yl)furo[3,2- d]pyrimidine-6-carboxamide | 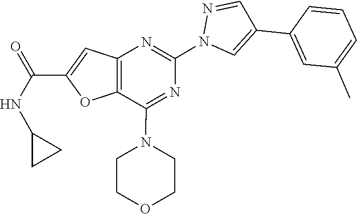 [SMILES: Cc1cccc(-c2cnn(-c3nc(N4CCOCC4)c4oc(C(=O)NC5CC5)cc4n3)c2)c1] |
| 101 | N-ethyl-4-morpholino-2-(4-(m-tolyl)- 1H-pyrazol-1-yl)furo[3,2-d]pyrimidine- 6-carboxamide | 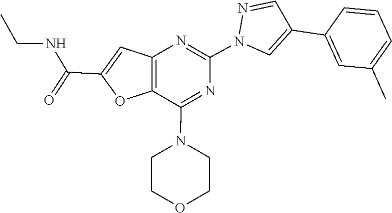 [SMILES: CCNC(=O)c1cc2nc(-n3cc(-c4cccc(C)c4)cn3)nc(N3CCOCC3)c2o1] |
| 102 | 4-morpholino-6-(pyridin-3-yl)-2-(4-(m- tolyl)-1H-pyrazol-1-yl)furo[3,2- d]pyrimidine | 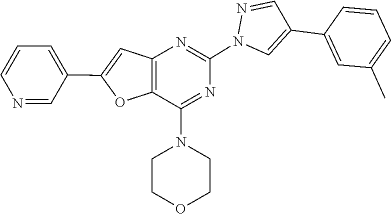 [SMILES: Cc1cccc(-c2cnn(-c3nc(N4CCOCC4)c4oc(-c5cccnc5)cc4n3)c2)c1] |
| 103 | 4-morpholino-6-(pyridin-2-yl)-2-(4-(m- tolyl)-1H-pyrazol-1-yl)furo[3,2- d]pyrimidine | 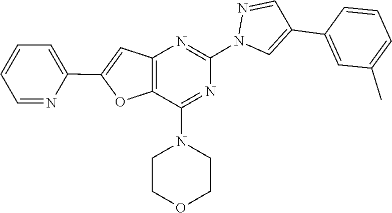 [SMILES: Cc1cccc(-c2cnn(-c3nc(N4CCOCC4)c4oc(-c5ccccn5)cc4n3)c2)c1] |
| 104 | 4-morpholino-N-(oxetan-3-yl)-2-(3-(m- tolyl)-1H-pyrazol-1-yl)furo[3,2- d]pyrimidine-6-carboxamide | 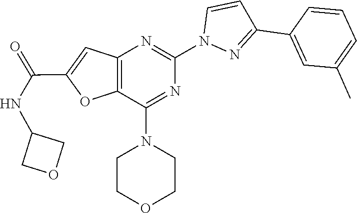 [SMILES: Cc1cccc(-c2ccn(-c3nc(N4CCOCC4)c4oc(C(=O)NC5COC5)cc4n3)n2)c1] |
| 105 | 4-morpholino-N-(oxetan-3-ylmethyl)-2- (3-(m-tolyl)-1H-pyrazol-1-yl)furo[3,2- d]pyrimidine-6-carboxamide | 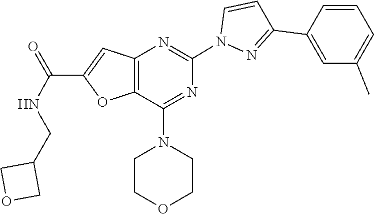 [SMILES: Cc1cccc(-c2ccn(-c3nc(N4CCOCC4)c4oc(C(=O)NCC5COC5)cc4n3)n2)c1] |
| 106 | 4-morpholino-N-(oxetan-3-yl)-2-(4-(m- tolyl)-1H-pyrazol-1-yl)furo[3,2- d]pyrimidine-6-carboxamide | 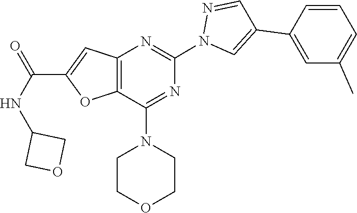 [SMILES: Cc1cccc(-c2cnn(-c3nc(N4CCOCC4)c4oc(C(=O)NC5COC5)cc4n3)c2)c1] |
| 107 | 4-morpholino-N-(oxetan-3-ylmethyl)-2- (4-(m-tolyl)-1H-pyrazol-1-yl)furo[3,2- d]pyrimidine-6-carboxamide | 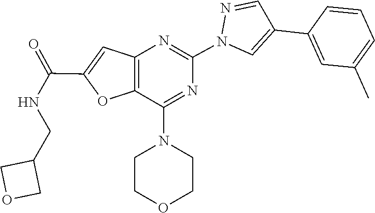 [SMILES: Cc1cccc(-c2cnn(-c3nc(N4CCOCC4)c4oc(C(=O)NCC5COC5)cc4n3)c2)c1] |
| 108 | N-(4-(dimethylamino)butyl)-4- morpholino-2-(3-(m-tolyl)-1H-pyrazol- 1-yl)furo[3,2-d]pyrimidine-6- carboxamide | 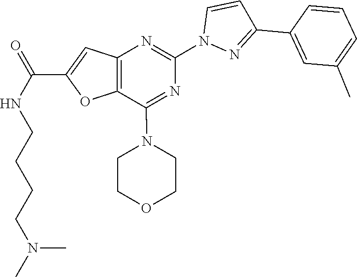 [SMILES: Cc1cccc(-c2ccn(-c3nc(N4CCOCC4)c4oc(C(=O)NCCCCN(C)C)cc4n3)n2)c1] |
| 109 | N-(3-(dimethylamino)propyl)-4- morpholino-2-(3-(m-tolyl)-1H-pyrazol- 1-yl)furo[3,2-d]pyrimidine-6- carboxamide | 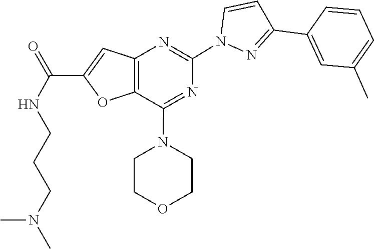 [SMILES: Cc1cccc(-c2ccn(-c3nc(N4CCOCC4)c4oc(C(=O)NCCCN(C)C)cc4n3)n2)c1] |
| 110 | N-(4-(dimethylamino)butyl)-4- morpholino-2-(4-(m-tolyl)-1H-pyrazol- 1-yl)furo[3,2-d]pyrimidine-6- carboxamide | 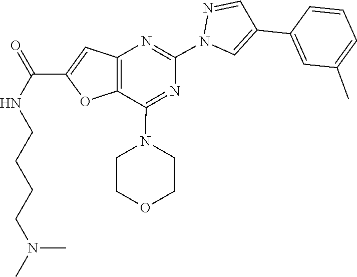 [SMILES: Cc1cccc(-c2cnn(-c3nc(N4CCOCC4)c4oc(C(=O)NCCCCN(C)C)cc4n3)c2)c1] |
| 111 | N-(3-(dimethylamino)propyl)-4- morpholino-2-(4-(m-tolyl)-1H-pyrazol- 1-yl)furo[3,2-d]pyrimidine-6- carboxamide | 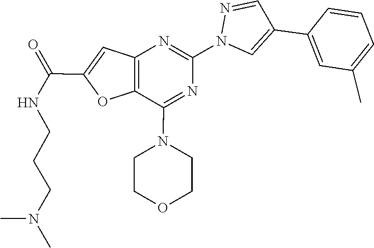 [SMILES: Cc1cccc(-c2cnn(-c3nc(N4CCOCC4)c4oc(C(=O)NCCCN(C)C)cc4n3)c2)c1] |
| 112 | 4-morpholino-6-(pyridin-2-yl)-2-(3-(m- tolyl)-1H-pyrazol-1-yl)-7- (trifluoromethyl)furo[3,2-d]pyrimidine | 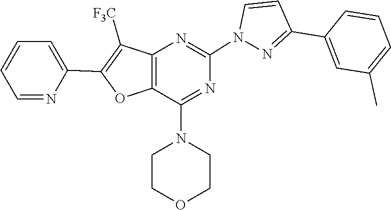 [SMILES: Cc1cccc(-c2ccn(-c3nc(N4CCOCC4)c4oc(-c5ccccn5)c(C(F)(F)F)c4n3)n2)c1] |
| 113 | (S)-N-(1-cyclopropylethyl)-4- morpholino-2-(4-(m-tolyl)-1H-pyrazol- 1-yl)furo[3,2-d]pyrimidine-6- carboxamide | 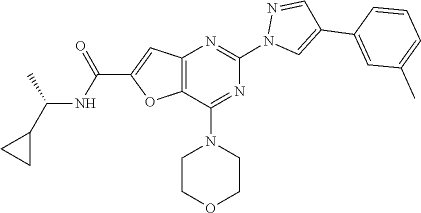 [SMILES: Cc1cccc(-c2cnn(-c3nc(N4CCOCC4)c4oc(C(=O)N[C@@H](C)C5CC5)cc4n3)c2)c1] |
| 114 | (S)-N-(1-cyclopropylethyl)-4- morpholino-2-(4-phenyl-1H-pyrazol-1- yl)furo[3,2-d]pyrimidine-6-carboxamide | 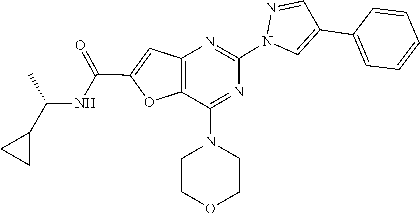 [SMILES: C[C@H](NC(=O)c1cc2nc(-n3cc(-c4ccccc4)cn3)nc(N3CCOCC3)c2o1)C1CC1] |
| 115 | N-(cyclopropylmethyl)-4-morpholino-2- (4-(m-tolyl)-1H-pyrazol-1-yl)furo[3,2- d]pyrimidine-6-carboxamide | 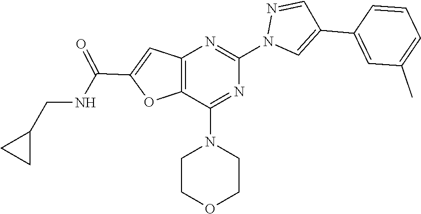 [SMILES: Cc1cccc(-c2cnn(-c3nc(N4CCOCC4)c4oc(C(=O)NCC5CC5)cc4n3)c2)c1] |
| 116 | N-(cyclopropylmethyl)-4-morpholino-2- (4-phenyl-1H-pyrazol-1-yl)furo[3,2- d]pyrimidine-6-carboxamide | 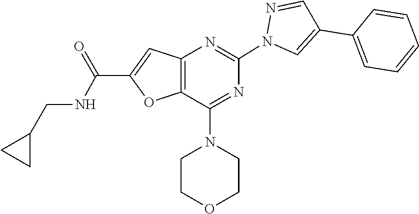 [SMILES: O=C(NCC1CC1)c1cc2nc(-n3cc(-c4ccccc4)cn3)nc(N3CCOCC3)c2o1] |
| 117 | N-(2-methoxyethyl)-4-morpholino-2-(4- (m-tolyl)-1H-pyrazol-1-yl)furo[3,2- d]pyrimidine-6-carboxamide | 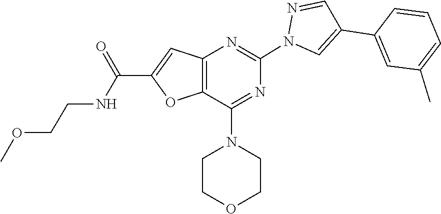 [SMILES: COCCNC(=O)c1cc2nc(-n3cc(-c4cccc(C)c4)cn3)nc(N3CCOCC3)c2o1] |
| 118 | N-(2-methoxyethyl)-4-morpholino-2-(4- phenyl-1H-pyrazol-1-yl)furo[3,2- d]pyrimidine-6-carboxamide | 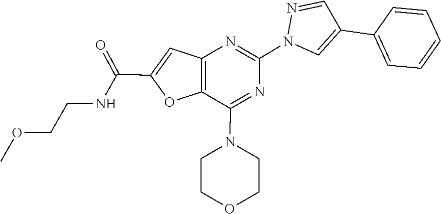 [SMILES: COCCNC(=O)c1cc2nc(-n3cc(-c4ccccc4)cn3)nc(N3CCOCC3)c2o1] |
| 119 | N-(3-(methylsulfonyl)propyl)-4- morpholino-2-(4-(m-tolyl)-1H-pyrazol- 1-yl)furo[3,2-d]pyrimidine-6- carboxamide | 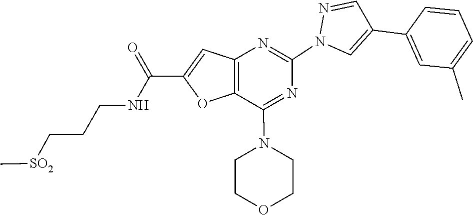 [SMILES: Cc1cccc(-c2cnn(-c3nc(N4CCOCC4)c4oc(C(=O)NCCCS(C)(=O)=O)cc4n3)c2)c1] |
| 120 | N-(3-(N,N-dimethylsulfamoyl)propyl)- 4-morpholino-2-(4-(m-tolyl)-1H- pyrazol-1-yl)furo[3,2-d]pyrimidine-6- carboxamide | 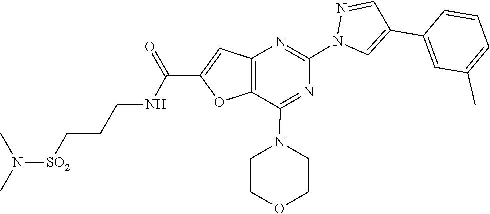 [SMILES: Cc1cccc(-c2cnn(-c3nc(N4CCOCC4)c4oc(C(=O)NCCCS(=O)(=O)N(C)C)cc4n3)c2)c1] |
| 121 | N-(1-methylpiperidin-4-yl)-4- morpholino-2-(4-(m-tolyl)-1H-pyrazol- 1-yl)furo[3,2-d]pyrimidine-6- carboxamide | 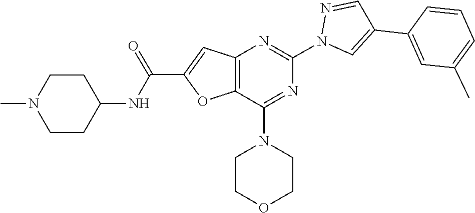 [SMILES: Cc1cccc(-c2cnn(-c3nc(N4CCOCC4)c4oc(C(=O)NC5CCN(C)CC5)cc4n3)c2)c1] |
| 122 | 7-methyl-4-morpholino-6-(pyridin-2- yl)-2-(3-(m-tolyl)-1H-pyrazol-1- yl)furo[3,2-d]pyrimidine | 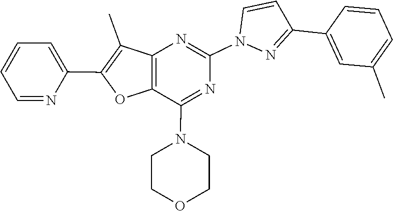 [SMILES: Cc1cccc(-c2ccn(-c3nc(N4CCOCC4)c4oc(-c5ccccn5)c(C)c4n3)n2)c1] |
| 123 | N-(1-methylpiperidin-3-yl)-4- moipholino-2-(4-(m-tolyl)-1H-pyrazol- 1-yl)furo[3,2-d]pyrimidine-6- carboxamide | 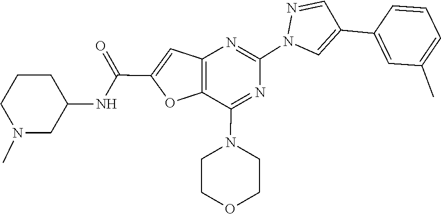 [SMILES: Cc1cccc(-c2cnn(-c3nc(N4CCOCC4)c4oc(C(=O)NC5CCCN(C)C5)cc4n3)c2)c1] |
| 124 | N-(1-methylpyrrolidin-3-yl)-4- morpholino-2-(4-(m-tolyl)-1H-pyrazol- 1-yl)furo[3,2-d]pyrimidine-6- carboxamide | 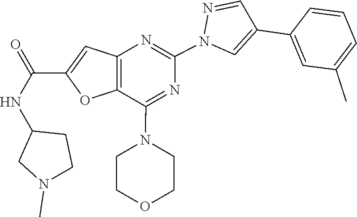 [SMILES: Cc1cccc(-c2cnn(-c3nc(N4CCOCC4)c4oc(C(=O)NC5CCN(C)C5)cc4n3)c2)c1] |
| 125 | 4-morpholino-N-(tetrahydro-2H-pyran- 4-yl)-2-(4-(m-tolyl)-1H-pyrazol-1- yl)furo[3,2-d]pyrimidine-6-carboxamide | 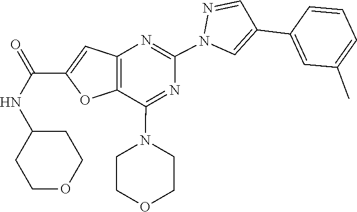 [SMILES: Cc1cccc(-c2cnn(-c3nc(N4CCOCC4)c4oc(C(=O)NC5CCOCC5)cc4n3)c2)c1] |
| 126 | 4-morpholino-N-(tetrahydrofuran-3-yl)- 2-(4-(m-tolyl)-1H-pyrazol-1-yl)furo[3,2- d]pyrimidine-6-carboxamide | 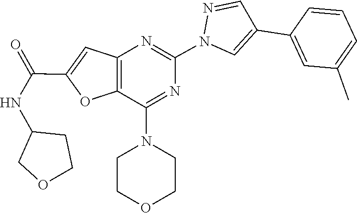 [SMILES: Cc1cccc(-c2cnn(-c3nc(N4CCOCC4)c4oc(C(=O)NC5CCOC5)cc4n3)c2)c1] |
| 127 | N-cyclopropyl-7-methyl-4-morpholino- 2-(4-(m-tolyl)-1H-pyrazol-1-yl)furo[3,2- d]pyrimidine-6-carboxamide | 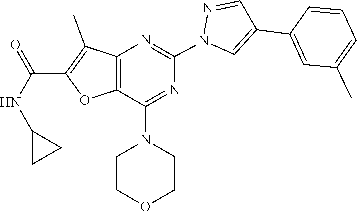 [SMILES: Cc1cccc(-c2cnn(-c3nc(N4CCOCC4)c4oc(C(=O)NC5CC5)c(C)c4n3)c2)c1] |
| 128 | N-(2-(N,N-dimethylsulfamoyl)ethyl)-4- morpholino-2-(4-(m-tolyl)-1H-pyrazol- 1-yl)furo[3,2-d]pyrimidine-6- carboxamide | 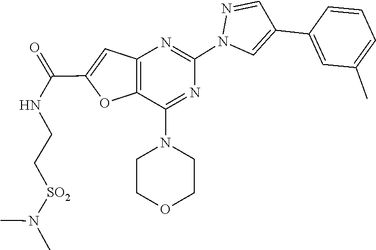 [SMILES: Cc1cccc(-c2cnn(-c3nc(N4CCOCC4)c4oc(C(=O)NCCS(=O)(=O)N(C)C)cc4n3)c2)c1] |
| 129 | N-(1-methylazepan-4-yl)-4-morpholino- 2-(4-(m-tolyl)-1H-pyrazol-1-yl)furo[3,2- d]pyrimidine-6-carboxamide | 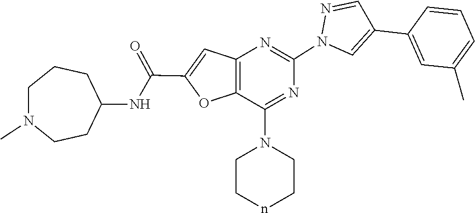 |
| 130 | N-(1-methylazocan-3-yl)-4-morpholino- 2-(4-(m-tolyl)-1H-pyrazol-1-yl)furo[3,2- d]pyrimidine-6-carboxamide | 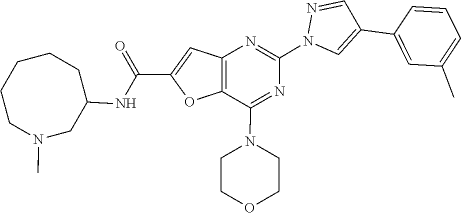 [SMILES: Cc1cccc(-c2cnn(-c3nc(N4CCOCC4)c4oc(C(=O)NC5CCCCCN(C)C5)cc4n3)c2)c1] |
| 131 | N-ethyl-4-morpholino(d₈)-2-(4-(m- tolyl)-1H-pyrazol-1-yl)furo[3,2- d]pyrimidine-6-carboxamide | 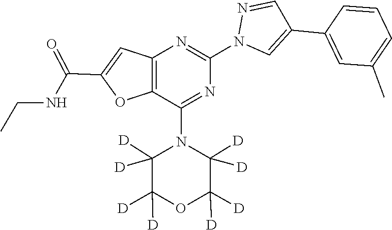 [SMILES: [2H]C1([2H])OC([2H])([2H])C([2H])([2H])N(c2nc(-n3cc(-c4cccc(C)c4)cn3)nc3cc(C(=O)NCC)oc23)C1([2H])[2H]] |
| 132 | 4-morpholino(ds)-6-(pyridin-3-yl)-2-(4- (m-tolyl)-1H-pyrazol-1-yl)furo[3,2- d]pyrimidine | 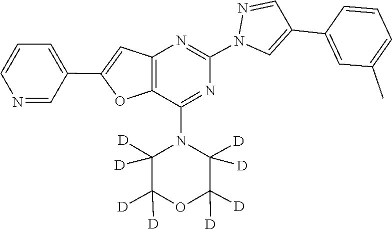 [SMILES: [2H]C1([2H])OC([2H])([2H])C([2H])([2H])N(c2nc(-n3cc(-c4cccc(C)c4)cn3)nc3cc(-c4cccnc4)oc23)C1([2H])[2H]] |
| 133 | 6-(pyridin-3-yl)-4-(2,2,6,6- tetrafluoromorpholino)-2-(4-(m-tolyl)- 1H-pyrazol-1-yl)furo[3,2-d]pyrimidine | 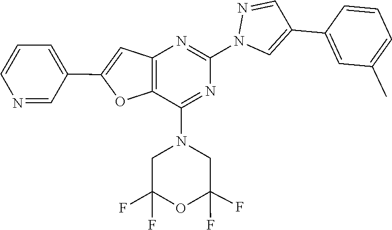 [SMILES: Cc1cccc(-c2cnn(-c3nc(N4CC(F)(F)OC(F)(F)C4)c4oc(-c5cccnc5)cc4n3)c2)c1] |
| 134 | N-ethyl-4-(2,2,6,6- tetrafluoromorpholino)-2-(4-(m-tolyl)- 1H-pyrazol-1-yl)furo[3,2-d]pyrimidine- 6-carboxamide | 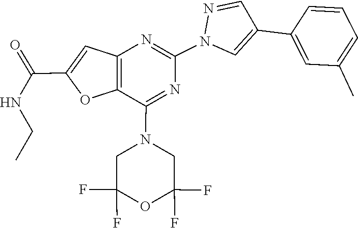 [SMILES: CCNC(=O)c1cc2nc(-n3cc(-c4cccc(C)c4)cn3)nc(N3CC(F)(F)OC(F)(F)C3)c2o1] |
| 135 | morpholino(4-morpholino-2-((5-phenyl- 1H-pyrazol-3-yl)amino)furo[3,2- d]pyrimidin-6-yl)methanone | 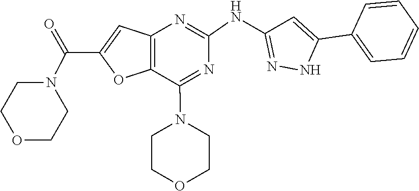 [SMILES: O=C(c1cc2nc(Nc3cc(-c4ccccc4)[nH]n3)nc(N3CCOCC3)c2o1)N1CCOCC1] |
| 136 | (4-methylpiperazin-1-yl)(4-morpholino- 2-((5-phenyl-1H-pyrazol-3- yl)amino)furo[3,2-d]pyrimidin-6- yl)methanone | 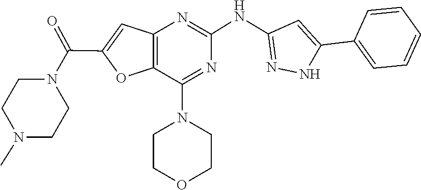 [SMILES: CN1CCN(C(=O)c2cc3nc(Nc4cc(-c5ccccc5)[nH]n4)nc(N4CCOCC4)c3o2)CC1] |
| 137 | N,N-dimethyl-4-morpholino-2-((5- phenyl-1H-pyrazol-3-yl)amino)furo[3,2- d]pyrimidine-6-carboxamide | 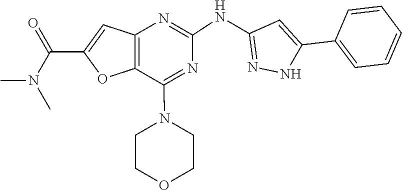 [SMILES: CN(C)C(=O)c1cc2nc(Nc3cc(-c4ccccc4)[nH]n3)nc(N3CCOCC3)c2o1] |
| 138 | N-methyl-4-morpholino-2-((5-phenyl- 1H-pyrazol-3-yl)amino)furo[3,2- d]pyrimidine-6-carboxamide | 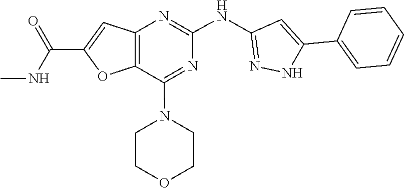 [SMILES: CNC(=O)c1cc2nc(Nc3cc(-c4ccccc4)[nH]n3)nc(N3CCOCC3)c2o1] |
| 139 | N-methoxy-N-methyl-4-morpholino-2- ((5-phenyl-1H-pyrazol-3- yl)amino)furo[3,2-d]pyrimidine-6- carboxamide | 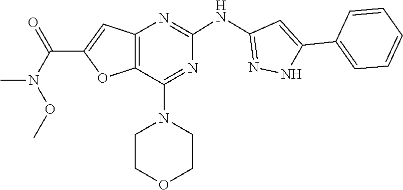 [SMILES: CON(C)C(=O)c1cc2nc(Nc3cc(-c4ccccc4)[nH]n3)nc(N3CCOCC3)c2o1] |
| 140 | N-(2-methoxyethyl)-4-morpholino-2- ((5-phenyl-1H-pyrazol-3- yl)amino)furo[3,2-d]pyrimidine-6- carboxamide | 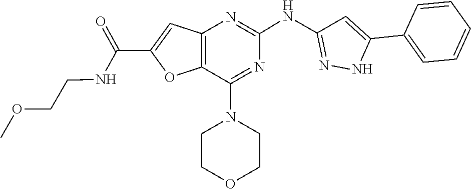 [SMILES: COCCNC(=O)c1cc2nc(Nc3cc(-c4ccccc4)[nH]n3)nc(N3CCOCC3)c2o1] |
| 141 | 4-morphohno-2-((5-phenyl-1H-pyrazol- 3-yl)amino)furo[3,2-d]pyrimidine-6- carboxylic acid | 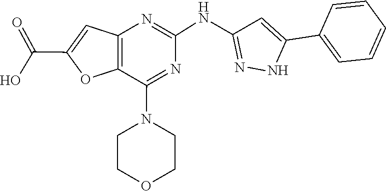 [SMILES: O=C(O)c1cc2nc(Nc3cc(-c4ccccc4)[nH]n3)nc(N3CCOCC3)c2o1] |
| 142 | 4-morpholino-N-(5-phenyl-1H-pyrazol- 3-yl)furo[3,2-d]pyrimidin-2-amine | 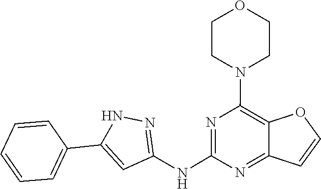 [SMILES: c1ccc(-c2cc(Nc3nc(N4CCOCC4)c4occc4n3)n[nH]2)cc1] |
| 143 | 4-morpholino-2-[4-(m-tolyl)pyrazol-1- yl]-6-pyrimidin-4-yl-furo[3,2- d]pyrimidine | 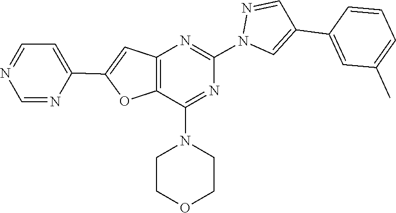 [SMILES: Cc1cccc(-c2cnn(-c3nc(N4CCOCC4)c4oc(-c5ccncn5)cc4n3)c2)c1] |
| 144 | 6-(3-methyl-1H-pyrazol-5-yl)-4- morpholino-2-[4-(m-tolyl)pyrazol-1- yl]furo[3,2-d]pyrimidine | 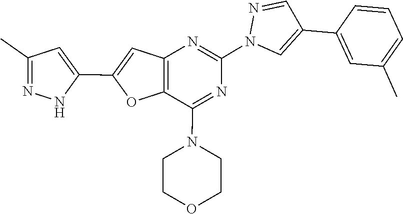 [SMILES: Cc1cccc(-c2cnn(-c3nc(N4CCOCC4)c4oc(-c5cc(C)n[nH]5)cc4n3)c2)c1] |
| 145 | N,N-dimethyl-2-[3-methyl-5-[4- morpholino-2-[4-(m-tolyl)pyrazol-1- yl]furo[3,2-d]pyrimidin-6-yl]pyrazol-1- yl]ethanamine | 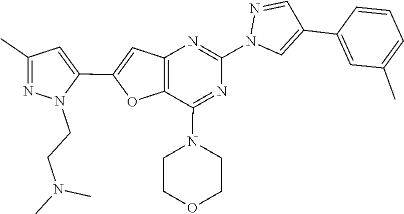 [SMILES: Cc1cccc(-c2cnn(-c3nc(N4CCOCC4)c4oc(-c5cc(C)nn5CCN(C)C)cc4n3)c2)c1] |
| 146 | N,N-dimethyl-2-[5-methyl-3-[4- morpholino-2-[4-(m-tolyl)pyrazol-1- yl]furo[3,2-d]pyrimidin-6-yl]pyrazol-1- yl]ethanamine | 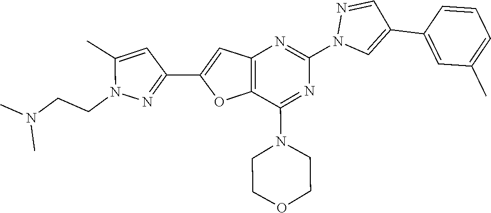 [SMILES: Cc1cccc(-c2cnn(-c3nc(N4CCOCC4)c4oc(-c5cc(C)n(CCN(C)C)n5)cc4n3)c2)c1] |
| 147 | N-cyclopropyl-4-morpholino-2-[4-(m- tolyl)pyrazol-1-yl]-7- (trifluoromethyl)furo[3,2-d]pyrimidine- 6-carboxamide | 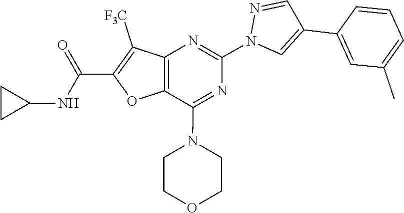 [SMILES: Cc1cccc(-c2cnn(-c3nc(N4CCOCC4)c4oc(C(=O)NC5CC5)c(C(F)(F)F)c4n3)c2)c1] |
| 148 | 4-[2-[3-(m-tolyl)pyrazol-1-yl]-6-(2- pyridyl)furo[3,2-d]pyrimidin-4- yl]morpholin-3-one | 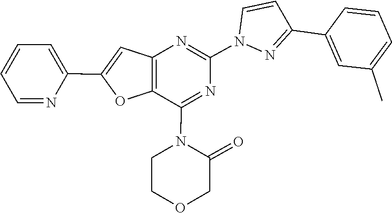 [SMILES: Cc1cccc(-c2ccn(-c3nc(N4CCOCC4=O)c4oc(-c5ccccn5)cc4n3)n2)c1] |
| 149 | N-ethyl-2-[4-(m-tolyl)pyrazol-1-yl]-4- (3-oxomorpholin-4-yl)furo[3,2- d]pyrimidine-6-carboxamide | 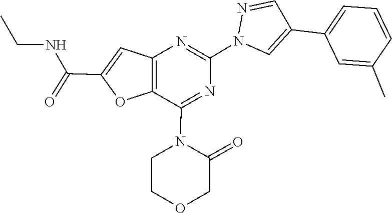 [SMILES: CCNC(=O)c1cc2nc(-n3cc(-c4cccc(C)c4)cn3)nc(N3CCOCC3=O)c2o1] |
| 150 | N-(2-methylsulfonylethyl)-4- morpholino-2-(4-phenylpyrazol-1- yl)furo[3,2-d]pyrimidine-6-carboxamide | 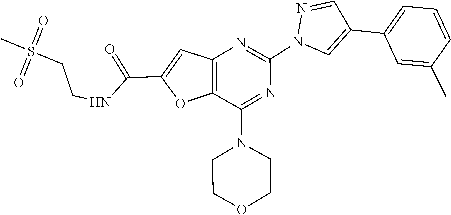 [SMILES: Cc1cccc(-c2cnn(-c3nc(N4CCOCC4)c4oc(C(=O)NCCS(C)(=O)=O)cc4n3)c2)c1] |
| 151 | N-(1-methyl-4-piperidyl)-4-morpholino- 2-(3-phenylpyrazol-1-yl)furo[3,2- d]pyrimidine-6-carboxamide | 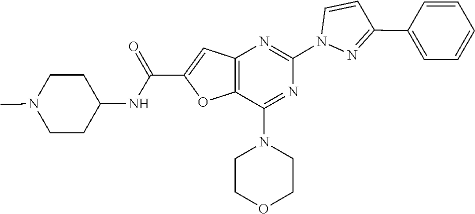 [SMILES: CN1CCC(NC(=O)c2cc3nc(-n4ccc(-c5ccccc5)n4)nc(N4CCOCC4)c3o2)CC1] |
| 152 | N-ethyl-4-morpholino-2-(3-phenyl-1H- pyrazol-1-yl)furo[3,2-d]pyrimidine-6- carboxamide | 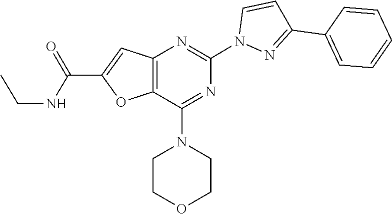 [SMILES: CCNC(=O)c1cc2nc(-n3ccc(-c4ccccc4)n3)nc(N3CCOCC3)c2o1] |
| 153 | N-cyclopropyl-4-morpholino-2-(3- phenylpyrazol-1-yl)furo[3,2- d]pyrimidine-6-carboxamide | 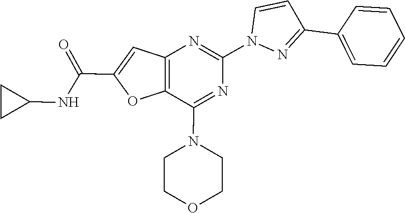 [SMILES: O=C(NC1CC1)c1cc2nc(-n3ccc(-c4ccccc4)n3)nc(N3CCOCC3)c2o1] |
| 154 | N-ethyl-4-morpholino-2-(4- phenylpyrazol-1-yl)furo[3,2- d]pyrimidine-6-carboxamide | 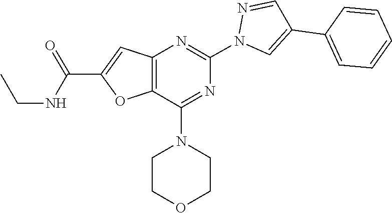 [SMILES: CCNC(=O)c1cc2nc(-n3cc(-c4ccccc4)cn3)nc(N3CCOCC3)c2o1] |
| 155 | 4-morpholino-2-(3-phenylpyrazol-1-yl)- 6-(2-pyridyl)furo[3,2-d]pyrimidine | 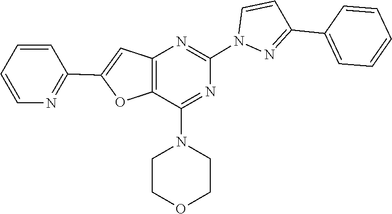 [SMILES: c1ccc(-c2ccn(-c3nc(N4CCOCC4)c4oc(-c5ccccn5)cc4n3)n2)cc1] |
| 156 | 4-morpholino-2-(3-phenylpyrazol-1-yl)- 6-(3-pyridyl)furo[3,2-d]pyrimidine | 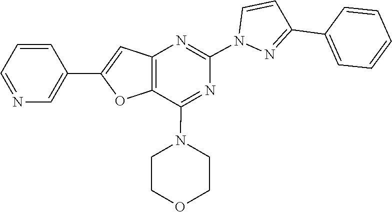 [SMILES: c1ccc(-c2ccn(-c3nc(N4CCOCC4)c4oc(-c5cccnc5)cc4n3)n2)cc1] |
| 157 | N-(1-methyl-4-piperidyl)-4-morpholino- 2-(4-phenylpyrazol-1-yl)furo[3,2- d]pyrimidine-6-carboxamide | 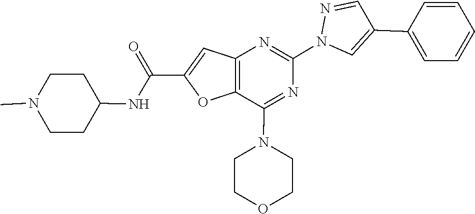 [SMILES: CN1CCC(NC(=O)c2cc3nc(-n4cc(-c5ccccc5)cn4)nc(N4CCOCC4)c3o2)CC1] |
| 158 | 4-morpholino-2-(3-phenylpyrazol-1-yl)- 6-(4-pyridyl)furo[3,2-d]pyrimidine | 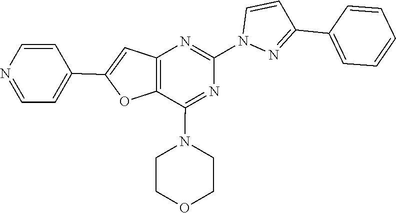 [SMILES: c1ccc(-c2ccn(-c3nc(N4CCOCC4)c4oc(-c5ccncc5)cc4n3)n2)cc1] |
| 159 | 4-morpholino-N-(oxetan-3-ylmethyl)-2- (3-phenylpyrazol-1-yl)furo[3,2- d]pyrimidine-6-carboxamide | 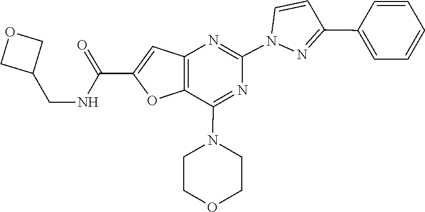 [SMILES: O=C(NCC1COC1)c1cc2nc(-n3ccc(-c4ccccc4)n3)nc(N3CCOCC3)c2o1] |
| 160 | 4-morpholino-2-(4-phenylpyrazol-1-yl)- 6-(3-pyridyl)furo[3,2-d]pyrimidine | 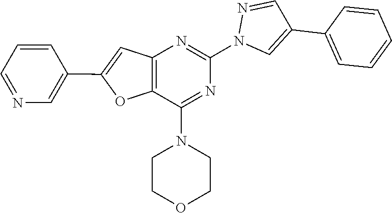 [SMILES: c1ccc(-c2cnn(-c3nc(N4CCOCC4)c4oc(-c5cccnc5)cc4n3)c2)cc1] |
| 161 | 4-morpholino-2-(4-phenylpyrazol-1-yl)- 6-(2-pyridyl)furo[3,2-d]pyrimidine | 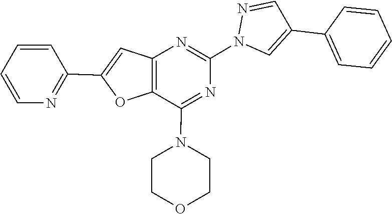 [SMILES: c1ccc(-c2cnn(-c3nc(N4CCOCC4)c4oc(-c5ccccn5)cc4n3)c2)cc1] |
| 162 | 6-(3-methyl-1H-pyrazol-5-yl)-4- morpholino-2-(3-phenylpyrazol-1- yl)furo[3,2-d]pyrimidine | 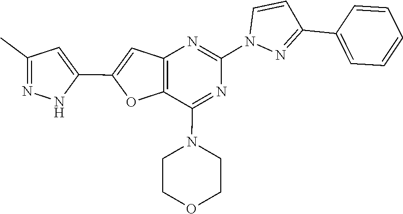 [SMILES: Cc1cc(-c2cc3nc(-n4ccc(-c5ccccc5)n4)nc(N4CCOCC4)c3o2)[nH]n1] |
| 163 | 6-(3-methyl-1H-pyrazol-5-yl)-4- morpholino-2-(4-phenylpyrazol-1- yl)furo[3,2-d]pyrimidine | 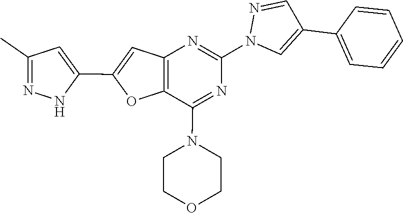 [SMILES: Cc1cc(-c2cc3nc(-n4cc(-c5ccccc5)cn4)nc(N4CCOCC4)c3o2)[nH]n1] |
| 164 | 4-morpholino-N-(oxetan-3-ylmethyl)-2- (4-phenylpyrazol-1-yl)furo[3,2- d]pyrimidine-6-carboxamide | 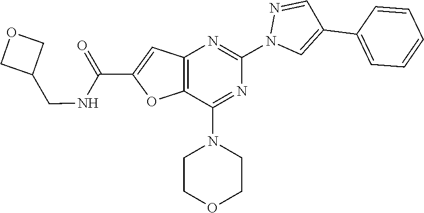 [SMILES: O=C(NCC1COC1)c1cc2nc(-n3cc(-c4ccccc4)cn3)nc(N3CCOCC3)c2o1] |
| 165 | N-cyclopropyl-4-morpholino-2-(4- phenylpyrazol-1-yl)furo[3,2- d]pyrimidine-6-carboxamide | 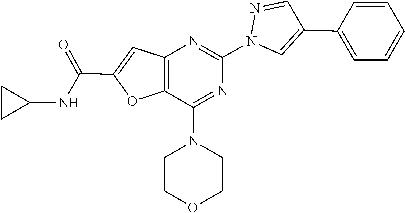 [SMILES: O=C(NC1CC1)c1cc2nc(-n3cc(-c4ccccc4)cn3)nc(N3CCOCC3)c2o1] |
| 166 | N-(cyclopropylmethyl)-4-morpholino-2- (3-phenylpyrazol-1-yl)furo[3,2- d]pyrimidine-6-carboxamide | 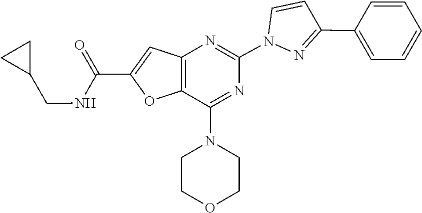 [SMILES: O=C(NCC1CC1)c1cc2nc(-n3ccc(-c4ccccc4)n3)nc(N3CCOCC3)c2o1] |
| 167 | N-[4-(dimethylamino)-1-methyl-butyl]- 4-morpholino-2-(4-phenylpyrazol-1- yl)furo[3,2-d]pyrimidine-6-carboxamide | 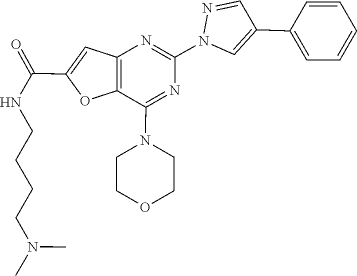 [SMILES: CN(C)CCCCNC(=O)c1cc2nc(-n3cc(-c4ccccc4)cn3)nc(N3CCOCC3)c2o1] |
| 168 | N-(3-(dimethylamino)propyl)-4- morpholino-2-(4-phenyl-1H-pyrazol-1- yl)furo[3,2-d]pyrimidine-6-carboxamide | 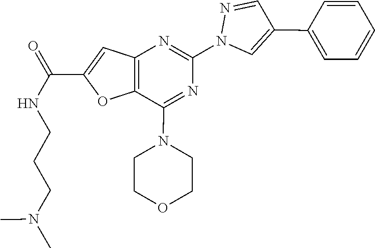 [SMILES: CN(C)CCCNC(=O)c1cc2nc(-n3cc(-c4ccccc4)cn3)nc(N3CCOCC3)c2o1] |
| 169 | N-(1-methyl-3-piperidyl)-4-morpholino- 2-(4-phenylpyrazol-1-yl)furo[3,2- d]pyrimidine-6-carboxamide | 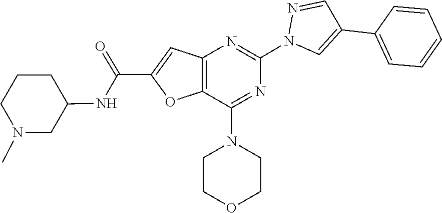 [SMILES: CN1CCCC(NC(=O)c2cc3nc(-n4cc(-c5ccccc5)cn4)nc(N4CCOCC4)c3o2)C1] |
| 170 | N-(1-methylpyrrolidin-3-yl)-4- morpholino-2-(4-phenylpyrazol-1- yl)furo[3,2-d]pyrimidine-6-carboxamide | 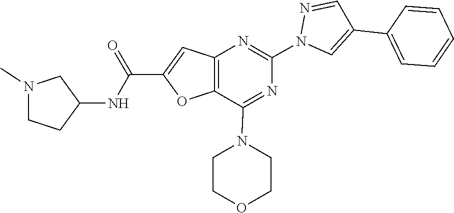 [SMILES: CN1CCC(NC(=O)c2cc3nc(-n4cc(-c5ccccc5)cn4)nc(N4CCOCC4)c3o2)C1] |
| 171 | 4-morpholino-2-(4-phenylpyrazol-1-yl)- N-tetrahydropyran-4-yl-furo[3,2- d]pyrimidine-6-carboxamide | 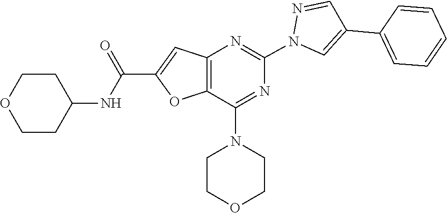 [SMILES: O=C(NC1CCOCC1)c1cc2nc(-n3cc(-c4ccccc4)cn3)nc(N3CCOCC3)c2o1] |
| 172 | 4-morpholino-2-(4-phenylpyrazol-1-yl)- N-tetrahydrofuran-3-yl-furo[3,2- d]pyrimidine-6-carboxamide | 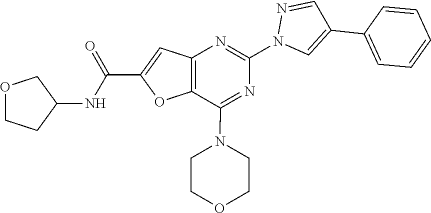 [SMILES: O=C(NC1CCOC1)c1cc2nc(-n3cc(-c4ccccc4)cn3)nc(N3CCOCC3)c2o1] |
| 173 | 6-(3-methyl-1H-pyrazol-5-yl)-4- morpholino-2-[3-(m-lolyl)pyrazol-1- yl]furo[3,2-d]pyrimidine | 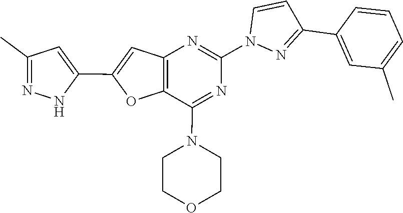 [SMILES: Cc1cccc(-c2ccn(-c3nc(N4CCOCC4)c4oc(-c5cc(C)n[nH]5)cc4n3)n2)c1] |
| 174 | 4-morpholino-N-(oxetan-3-yl)-2-(4- phenylpyrazol-1-yl)furo[3,2- d]pyrimidine-6-carboxamide | 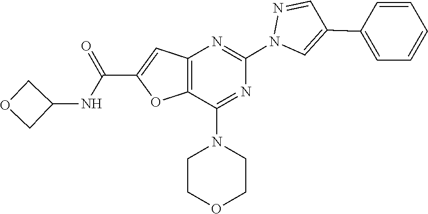 [SMILES: O=C(NC1COC1)c1cc2nc(-n3cc(-c4ccccc4)cn3)nc(N3CCOCC3)c2o1] |
| 175 | 4-morpholino-N-(oxetan-3-yl)-2-(3- phenylpyrazol-1-yl)furo[3,2- d]pyrimidine-6-carboxamide | 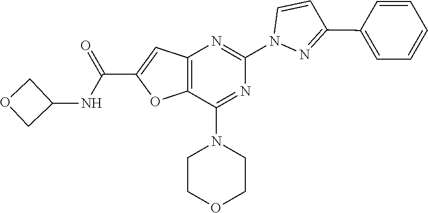 [SMILES: O=C(NC1COC1)c1cc2nc(-n3ccc(-c4ccccc4)n3)nc(N3CCOCC3)c2o1] |
| 176 | N-(1-methylazepan-4-yl)-4-morpholino- 2-(4-phenylpyrazol-1-yl)furo[3,2- d]pyrimidine-6-carboxamide | 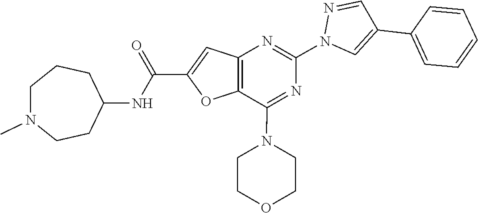 [SMILES: CN1CCCC(NC(=O)c2cc3nc(-n4cc(-c5ccccc5)cn4)nc(N4CCOCC4)c3o2)CC1] |
| 177 | N,N-dimethyl-2-[5-methyl-3-[4- morpholino-2-(3-phenylpyrazol-1- yl)furo[3,2-d]pyrimidin-6-yl]pyrazol-1- yl]ethanamine | 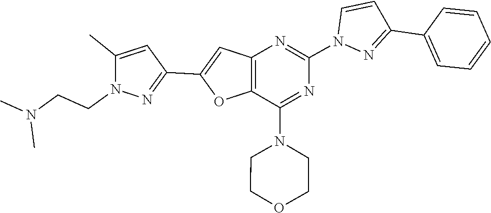 [SMILES: Cc1cc(-c2cc3nc(-n4ccc(-c5ccccc5)n4)nc(N4CCOCC4)c3o2)nn1CCN(C)C] |
| 178 | N,N-dimethyl-2-[5-methyl-3-[4- morpholino-2-[3-(m-tolyl)pyrazol-1- yl]furo[3,2-d]pyrimidin-6-yl]pyrazol-1- yl] ethanamine | 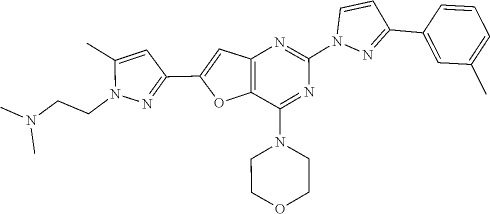 [SMILES: Cc1cccc(-c2ccn(-c3nc(N4CCOCC4)c4oc(-c5cc(C)n(CCN(C)C)n5)cc4n3)n2)c1] |
| 179 | 7-methyl-4-morpholino-2-(3- phenylpyrazol-1-yl)-6-(2- pyridyl)furo[3,2-d]pyrimidine | 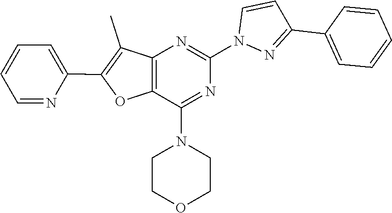 [SMILES: Cc1c(-c2ccccn2)oc2c(N3CCOCC3)nc(-n3ccc(-c4ccccc4)n3)nc12] |
| 180 | 7-methyl-4-morpholino-2-(3- phenylpyrazol-1-yl)-6-(3- pyridyl)furo[3,2-d]pyrimidine | 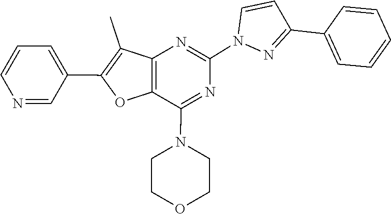 [SMILES: Cc1c(-c2cccnc2)oc2c(N3CCOCC3)nc(-n3ccc(-c4ccccc4)n3)nc12] |
| 181 | 7-methyl-4-morpholino-2-(3- phenylpyrazol-1-yl)-6-(4- pyridyl)furo[3,2-d]pyrimidine | 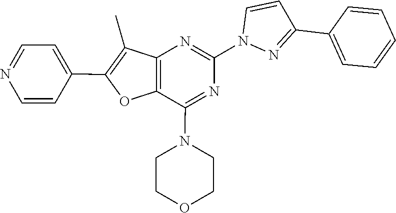 [SMILES: Cc1c(-c2ccncc2)oc2c(N3CCOCC3)nc(-n3ccc(-c4ccccc4)n3)nc12] |
| 182 | 7-methyl-6-(3-methyl-1H-pyrazol-5-yl)- 4-morpholino-2-(3-phenylpyrazol-1- yl)furo[3,2-d]pyrimidine | 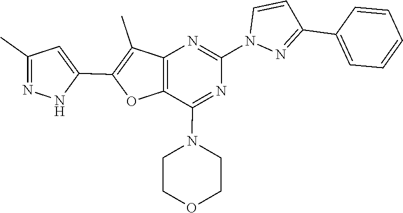 [SMILES: Cc1cc(-c2oc3c(N4CCOCC4)nc(-n4ccc(-c5ccccc5)n4)nc3c2C)[nH]n1] |
| 183 | N,N-dimethyl-2-[5-methyl-3-[4- morpholino-2-(4-phenylpyrazol-1- yl)furo[3,2-d]pyrimidin-6-yl]pyrazol-1- yl]ethanamine | 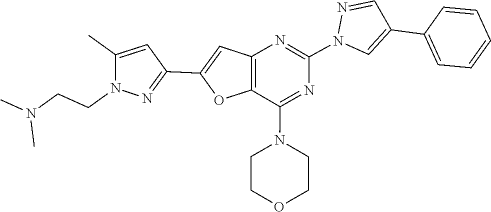 [SMILES: Cc1cc(-c2cc3nc(-n4cc(-c5ccccc5)cn4)nc(N4CCOCC4)c3o2)nn1CCN(C)C] |
| 184 | N-cyclopropyl-7-methyl-4-morpholino- 2-(3-phenylpyrazol-1-yl)furo[3,2- d]pyrimidine-6-carboxamide | 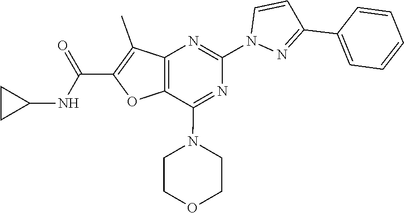 [SMILES: Cc1c(C(=O)NC2CC2)oc2c(N3CCOCC3)nc(-n3ccc(-c4ccccc4)n3)nc12] |
| 185 | 2-[3-(3-chlorophenyl)pyrazol-1-yl]-4- morpholino-6-(2-pyridyl)furo[3,2- d]pyrimidine | 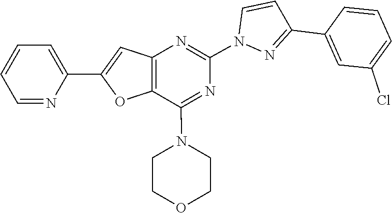 [SMILES: Clc1cccc(-c2ccn(-c3nc(N4CCOCC4)c4oc(-c5ccccn5)cc4n3)n2)c1] |
| 186 | 2-[3-(3-bromophenyl)pyrazol-1-yl]-4- morpholino-6-(2-pyridyl)furo[3,2- d]pyrimidine | 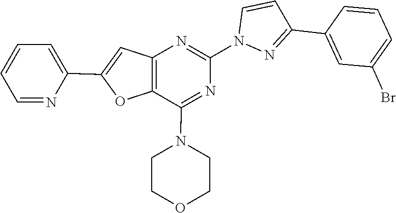 [SMILES: Brc1cccc(-c2ccn(-c3nc(N4CCOCC4)c4oc(-c5ccccn5)cc4n3)n2)c1] |
| 187 | 2-[3-(6-methyl-2-pyridyl)pyrazol-1-yl]- 4-morpholino-6-(2-pyridyl)furo[3,2- d]pyrimidine | 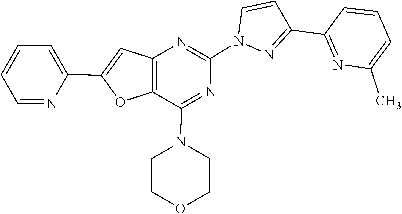 [SMILES: Cc1cccc(-c2ccn(-c3nc(N4CCOCC4)c4oc(-c5ccccn5)cc4n3)n2)n1] |
| 188 | 2-[3-(2-methyl-4-pyridyl)pyrazol-1-yl]- 4-morpholino-6-(2-pyridyl)furo[3,2- d]pyrimidine | 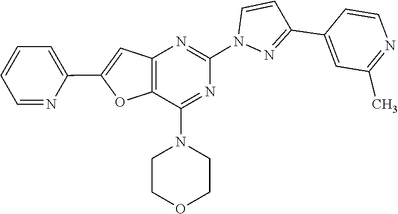 [SMILES: Cc1cc(-c2ccn(-c3nc(N4CCOCC4)c4oc(-c5ccccn5)cc4n3)n2)ccn1] |
| 189 | 6-(1-methylpyrazol-3-yl)-4-morpholino- 2-[3-(m-tolyl)pyrazol-1-yl]furo[3,2- d]pyrimidine | 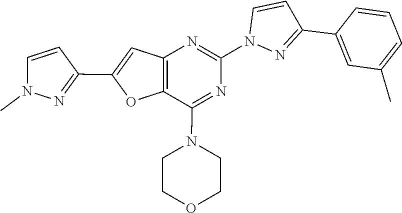 [SMILES: Cc1cccc(-c2ccn(-c3nc(N4CCOCC4)c4oc(-c5ccn(C)n5)cc4n3)n2)c1] |
| 190 | 6-(1-methylpyrazol-3-yl)-4-morpholino- 2-(4-phenylpyrazol-1-yl)furo[3,2- d]pyrimidine | 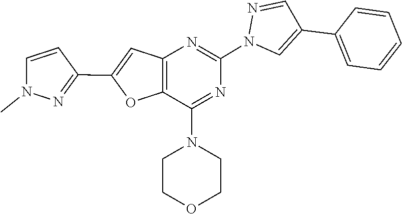 [SMILES: Cn1ccc(-c2cc3nc(-n4cc(-c5ccccc5)cn4)nc(N4CCOCC4)c3o2)n1] |
| 191 | 6-(1-methylpyrazol-3-yl)-4-morpholino- 2-(3-phenylpyrazol-1-yl)furo[3,2- d]pyrimidine | 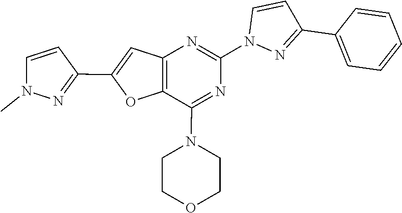 [SMILES: Cn1ccc(-c2cc3nc(-n4ccc(-c5ccccc5)n4)nc(N4CCOCC4)c3o2)n1] |
| 192 | 2-[4-(3-chlorophenyl)pyrazol-1-yl]-6-(1- methylpyrazol-3-yl)-4-morpholino- furo[3,2-d]pyrimidine | 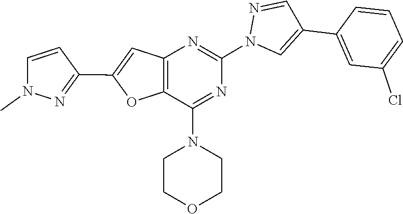 [SMILES: Cn1ccc(-c2cc3nc(-n4cc(-c5cccc(Cl)c5)cn4)nc(N4CCOCC4)c3o2)n1] |
| 193 | 2-[3-(3-chlorophenyl)pyrazol-1-yl]-6-(1- methylpyrazol-3-yl)-4-morpholino- furo[3,2-d]pyrimidine | 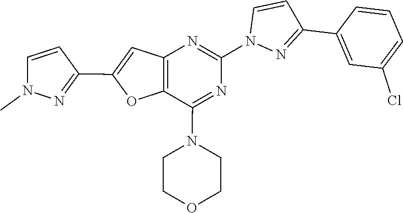 [SMILES: Cn1ccc(-c2cc3nc(-n4ccc(-c5cccc(Cl)c5)n4)nc(N4CCOCC4)c3o2)n1] |
| 194 | 2-(4-bromopyrazol-1-yl)-6-(1- methylpyrazol-3-yl)-4-morpholino- furo[3,2-d]pyrimidine | 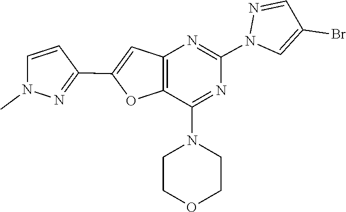 [SMILES: Cn1ccc(-c2cc3nc(-n4cc(Br)cn4)nc(N4CCOCC4)c3o2)n1] |
| 195 | 2-(3-bromopyrazol-1-yl)-6-(1- methylpyrazol-3-yl)-4-morpholino- furo[3,2-d]pyrimidine | 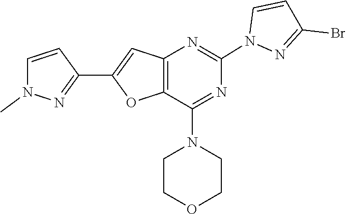 [SMILES: Cn1ccc(-c2cc3nc(-n4ccc(Br)n4)nc(N4CCOCC4)c3o2)n1] |
| 196 | N-[2-[5-methyl-3-[4-morpholino-2-[4- (m-tolyl)pyrazol-1-yl]furo[3,2- d]pyrimidin-6-yl]pyrazol-1- yl]ethyl]prop-2-enamide | 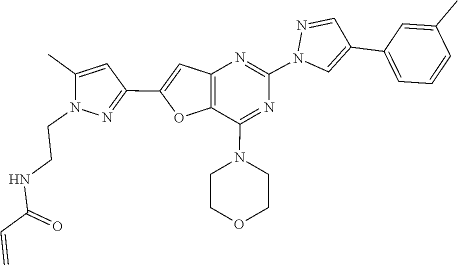 [SMILES: C=CC(=O)NCCn1nc(-c2cc3nc(-n4cc(-c5cccc(C)c5)cn4)nc(N4CCOCC4)c3o2)cc1C] |
| 197 | N-[2-[5-methyl-3-[4-morpholino-2-[4- (m-tolyl)pyrazol-1-yl]furo[3,2- d]pyrimidin-6-yl]pyrazol-1- yl]ethyl]acetamide | 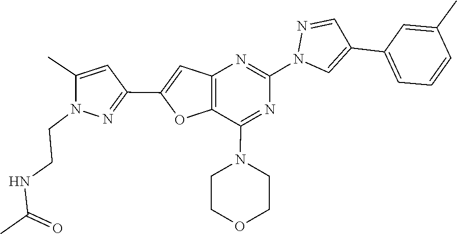 [SMILES: CC(=O)NCCn1nc(-c2cc3nc(-n4cc(-c5cccc(C)c5)cn4)nc(N4CCOCC4)c3o2)cc1C] |
| 198 | 2-chloro-N-[2-[5-methyl-3-[4- morpholino-2-[4-(m-tolyl)pyrazol-1- yl]furo[3,2-d]pyrimidin-6-yl]pyrazol-1- yl]ethyl]acetamide | 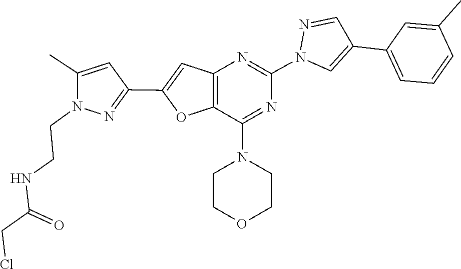 [SMILES: Cc1cccc(-c2cnn(-c3nc(N4CCOCC4)c4oc(-c5cc(C)n(CCNC(=O)CCl)n5)cc4n3)c2)c1] |
| 199 | 2-[3-(3-methoxyphenyl)pyrazol-1-yl]-4- morpholino-6-(2-pyridyl)furo[3,2- d]pyrimidine | 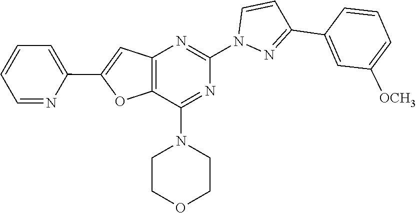 [SMILES: COc1cccc(-c2ccn(-c3nc(N4CCOCC4)c4oc(-c5ccccn5)cc4n3)n2)c1] |
| 200 | 2-[3-(5-methyl-3-pyridyl)pyrazol-1-yl]- 4-morpholino-6-(2-pyridyl)furo[3,2- d]pyrimidine | 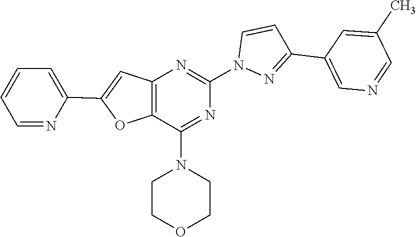 [SMILES: Cc1cncc(-c2ccn(-c3nc(N4CCOCC4)c4oc(-c5ccccn5)cc4n3)n2)c1] |
| 201 | 6-(1-methylpyrazol-3-yl)-4-morpholino- 2-[4-(m-tolyl)pyrazol-1-yl]furo[3,2- d]pyrimidine | 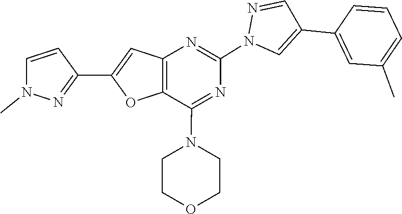 [SMILES: Cc1cccc(-c2cnn(-c3nc(N4CCOCC4)c4oc(-c5ccn(C)n5)cc4n3)c2)c1] |
| 202 | 1-[2-[5-methyl-3-[4-morpholino-2-[4- (m-tolyl)pyrazol-1-yl]furo[3,2- d]pyrimidin-6-yl]pyrazol-1- yl]ethyl]pyrrole-2,5-dione | 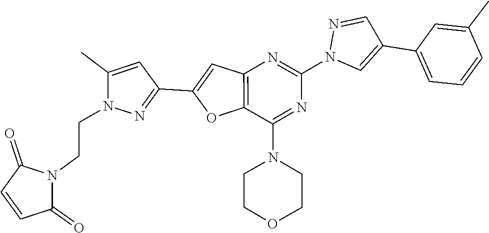 [SMILES: Cc1cccc(-c2cnn(-c3nc(N4CCOCC4)c4oc(-c5cc(C)n(CCN6C(=O)C=CC6=O)n5)cc4n3)c2)c1] |
| 203 | 4-morpholino-2-[3-(m-tolyl)pyrazol-1- yl]-6-thiazol-2-yl-furo[3,2-d]pyrimidine | 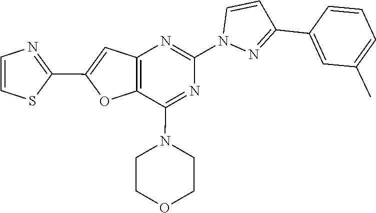 [SMILES: Cc1cccc(-c2ccn(-c3nc(N4CCOCC4)c4oc(-c5nccs5)cc4n3)n2)c1] |
| 204 | 6-(3-methylisoxazol-5-yl)-4- morpholino-2-[3-(m-tolyl)pyrazol-1- yl]furo[3,2-d]pyrimidine | 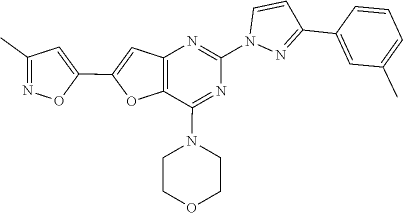 [SMILES: Cc1cccc(-c2ccn(-c3nc(N4CCOCC4)c4oc(-c5cc(C)no5)cc4n3)n2)c1] |
| 205 | 6-(1-methylpyrazol-4-yl)-4-morpholino- 2-[3-(m-tolyl)pyrazol-1-yl]furo[3,2- d]pyrimidine | 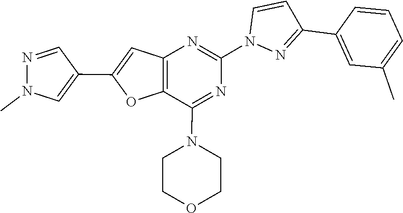 [SMILES: Cc1cccc(-c2ccn(-c3nc(N4CCOCC4)c4oc(-c5cnn(C)c5)cc4n3)n2)c1] |
| 206 | 6-(3-methylisoxazol-5-yl)-4- morpholino-2-(3-phenylpyrazol-1- yl)furo[3,2-d]pyrimidine | 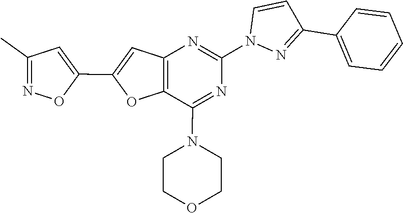 [SMILES: Cc1cc(-c2cc3nc(-n4ccc(-c5ccccc5)n4)nc(N4CCOCC4)c3o2)on1] |
| 207 | 4-morpholino-2-(3-phenylpyrazol-1-yl)- 6-thiazol-2-yl-furo[3,2-d]pyrimidine | 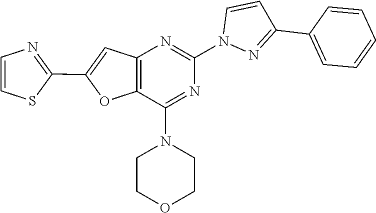 [SMILES: c1ccc(-c2ccn(-c3nc(N4CCOCC4)c4oc(-c5nccs5)cc4n3)n2)cc1] |
| 208 | 6-(1-methylpyrazol-4-yl)-4-morpholino- 2-(3-phenylpyrazol-1-yl)furo[3,2- d]pyrimidine | 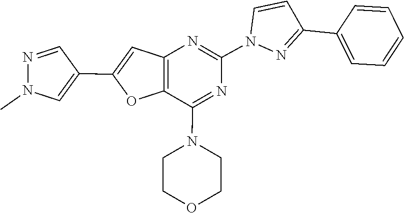 [SMILES: Cn1cc(-c2cc3nc(-n4ccc(-c5ccccc5)n4)nc(N4CCOCC4)c3o2)cn1] |
| 209 | 4-morpholino-2-(3-phenylpyrazol-1-yl)- 6-(1H-pyrazol-4-yl)furo[3,2- d]pyrimidine | 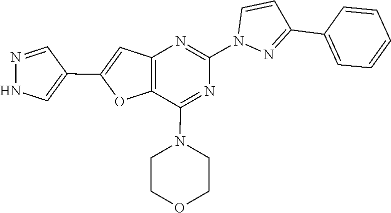 [SMILES: c1ccc(-c2ccn(-c3nc(N4CCOCC4)c4oc(-c5cn[nH]c5)cc4n3)n2)cc1] |
| 210 | 6-(3-methylisothiazol-5-yl)-4- morpholino-2-[3-(m-tolyl)pyrazol-1- yl]furo[3,2-d]pyrimidine | 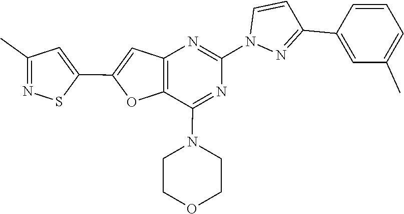 [SMILES: Cc1cccc(-c2ccn(-c3nc(N4CCOCC4)c4oc(-c5cc(C)ns5)cc4n3)n2)c1] |
| 211 | 6-(3-methylisothiazol-5-yl)-4- morpholino-2-(3-phenylpyrazol-1- yl)furo[3,2-d]pyrimidine | 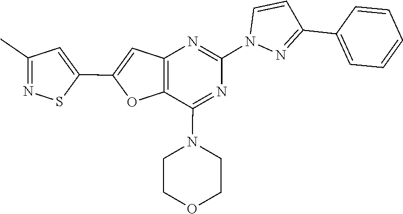 [SMILES: Cc1cc(-c2cc3nc(-n4ccc(-c5ccccc5)n4)nc(N4CCOCC4)c3o2)sn1] |
| 212 | 6-(2-methyl-1H-imidazol-5-yl)-4- morpholino-2-[3-(m-tolyl)pyrazol-1- yl]furo[3,2-d]pyrimidine | 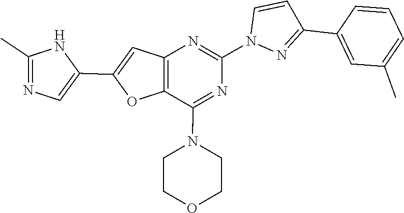 [SMILES: Cc1cccc(-c2ccn(-c3nc(N4CCOCC4)c4oc(-c5cnc(C)[nH]5)cc4n3)n2)c1] |
| 213 | 6-(2-methyl-1H-imidazol-5-yl)-4- morpholino-2-(3-phenylpyrazol-1- yl)furo[3,2-d]pyrimidine | 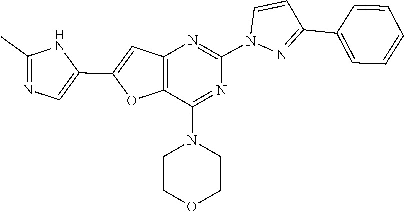 [SMILES: Cc1ncc(-c2cc3nc(-n4ccc(-c5ccccc5)n4)nc(N4CCOCC4)c3o2)[nH]1] |
| 214 | 4-morpholino-6-oxazol-2-yl-2-(3- phenylpyrazol-1-yl)furo[3,2- d]pyrimidine | 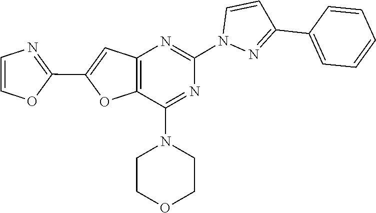 [SMILES: c1ccc(-c2ccn(-c3nc(N4CCOCC4)c4oc(-c5ncco5)cc4n3)n2)cc1] |
| 215 | 6-(1-methylpyrazol-3-yl)-4-morpholino- 2-[4-[3- (trideuteriomethyl)phenyl]pyrazol-1- yl]furo[3,2-d]pyrimidine | 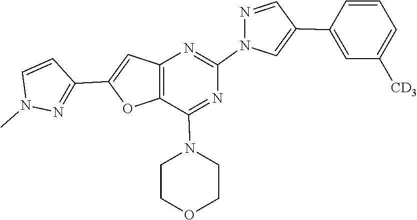 [SMILES: [2H]C([2H])([2H])c1cccc(-c2cnn(-c3nc(N4CCOCC4)c4oc(-c5ccn(C)n5)cc4n3)c2)c1] |
| 216 | 6-(1-methylpyrazol-3-yl)-4-morpholino- 2-[3-[3- (trideuteriomethyl)phenyl]pyrazol-1- yl]furo[3,2-d]pyrimidine | 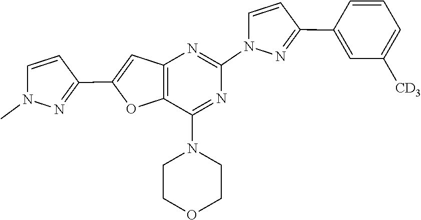 [SMILES: [2H]C([2H])([2H])c1cccc(-c2ccn(-c3nc(N4CCOCC4)c4oc(-c5ccn(C)n5)cc4n3)n2)c1] |
| 217 | 4-morpholino-6-(2-pyridyl)-2-[3-[3- (trideuteriomethyl)phenyl]pyrazol-1- yl]furo[3,2-d]pyrimidine | 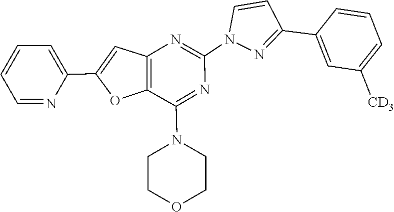 [SMILES: [2H]C([2H])([2H])c1cccc(-c2ccn(-c3nc(N4CCOCC4)c4oc(-c5ccccn5)cc4n3)n2)c1] |
| 218 | 4-morpholino-2-[3-(m-tolyl)pyrazol-1- yl]-6-oxazol-2-yl-furo[3,2-d]pyrimidine | 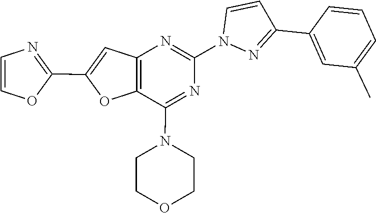 [SMILES: Cc1cccc(-c2ccn(-c3nc(N4CCOCC4)c4oc(-c5ncco5)cc4n3)n2)c1] |
| 219 | 4-morpholino-2-[3-(m-tolyl)pyrazol-1- yl]-6-(1H-pyrazol-4-yl)furo[3,2- d]pyrimidine | 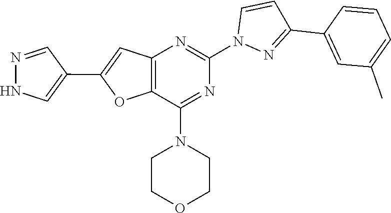 [SMILES: Cc1cccc(-c2ccn(-c3nc(N4CCOCC4)c4oc(-c5cn[nH]c5)cc4n3)n2)c1] |
| 220 | 2-[3-(3-bromophenyl)pyrazol-1-yl]-4- morpholino-N-tetrahydropyran-4-yl- furo[3,2-d]pyrimidine-6-carboxamide | 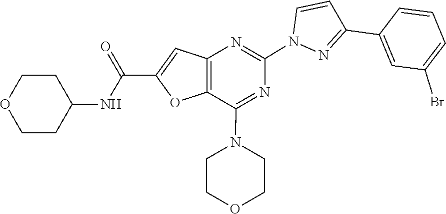 [SMILES: O=C(NC1CCOCC1)c1cc2nc(-n3ccc(-c4cccc(Br)c4)n3)nc(N3CCOCC3)c2o1] |
| 221 | 2-[3-(3-methoxyphenyl)pyrazol-1-yl]-4- morpholino-N-tetrahydropyran-4-yl- furo[3,2-d]pyrimidine-6-carboxamide | 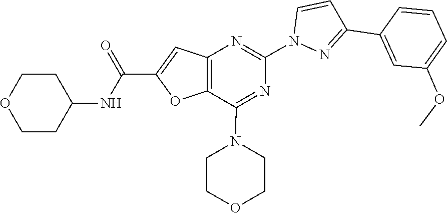 [SMILES: COc1cccc(-c2ccn(-c3nc(N4CCOCC4)c4oc(C(=O)NC5CCOCC5)cc4n3)n2)c1] |
| 222 | 4-morpholino-N-tetrahydropyran-4-yl-2- [4-[3-(trideuteriomethyl)phenyl]pyrazol- 1-yl]furo[3,2-d]pyrimidine-6- carboxamide | 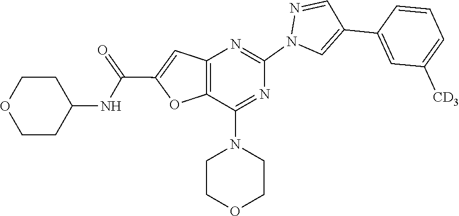 [SMILES: [2H]C([2H])([2H])c1cccc(-c2cnn(-c3nc(N4CCOCC4)c4oc(C(=O)NC5CCOCC5)cc4n3)c2)c1] |
| 223 | 2-[4-(3-methoxyphenyl)pyrazol-1-yl]-4- morpholino-N-tetrahydropyran-4-yl- furo[3,2-d]pyrimidine-6-carboxamrde | 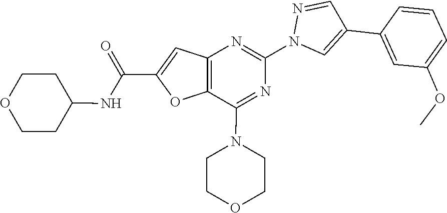 [SMILES: COc1cccc(-c2cnn(-c3nc(N4CCOCC4)c4oc(C(=O)NC5CCOCC5)cc4n3)c2)c1] |
| 224 | N,N-dimethyl-2-(1-methyl-3-(4- morpholino-2-(3-(m-tolyl)-1H-pyrazol- 1-yl)furo[3,2-d]pyrimidin-6-yl)-1H- pyrazol-5-yl)ethan-1-amine | 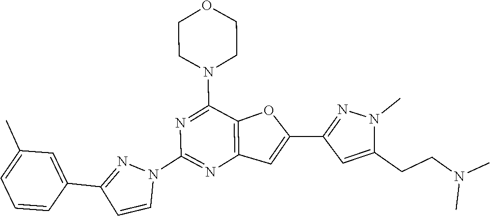 [SMILES: Cc1cccc(-c2ccn(-c3nc(N4CCOCC4)c4oc(-c5cc(CCN(C)C)n(C)n5)cc4n3)n2)c1] |
| 225 | 2-(4-(3-bromophenyl)-1H-pyrazol-1-yl)- 4-morpholino-N-(tetrahydro-2H-pyran- 4-yl)furo[3,2-d]pyrimidine-6- carboxamide | 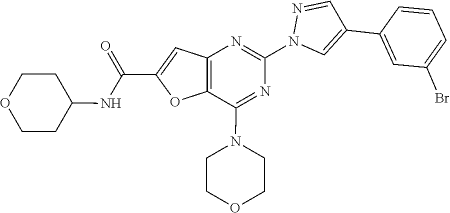 [SMILES: O=C(NC1CCOCC1)c1cc2nc(-n3cc(-c4cccc(Br)c4)cn3)nc(N3CCOCC3)c2o1] |
| 226 | 4-inorpholino-2-(4-phcnyl-1H-pyrazol- 1-yl)furo[3,2-d]pyrimidine | 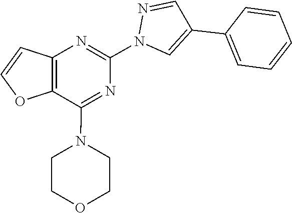 [SMILES: c1ccc(-c2cnn(-c3nc(N4CCOCC4)c4occc4n3)c2)cc1] |
| 227 | morpholino(4-morpholino-2-(3-(m- tolyl)-1H-pyrazol-1-yl)furo[3,2- d]pyrimidin-6-yl)methanone | 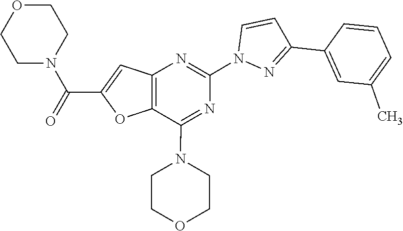 [SMILES: Cc1cccc(-c2ccn(-c3nc(N4CCOCC4)c4oc(C(=O)N5CCOCC5)cc4n3)n2)c1] |
| 228 | (4-methylpiperazin-1-yl)(4-morpholino- 2-(3-(m-tolyl)-1H-pyrazol-1-yl)furo[3,2- d]pyrimidin-6-yl)methanone | 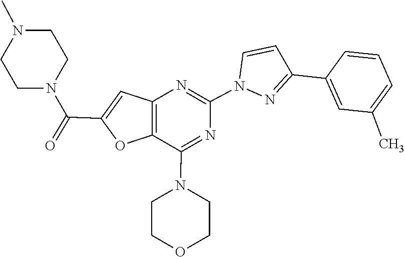 [SMILES: Cc1cccc(-c2ccn(-c3nc(N4CCOCC4)c4oc(C(=O)N5CCN(C)CC5)cc4n3)n2)c1] |
| 229 | N,N-dimethyl-4-morpholino-2-(3-(m- tolyl)-1H-pyrazol-1-yl)furo[3,2- d]pyrimidine-6-carboxamide | 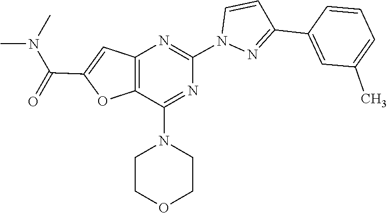 [SMILES: Cc1cccc(-c2ccn(-c3nc(N4CCOCC4)c4oc(C(=O)N(C)C)cc4n3)n2)c1] |
| 230 | N-methoxy-N-methyl-4-inorpholino-2- (3-(m-tolyl)-1H-pyrazol-1-yl)furo[3 2- d]pyrimidine-6-carboxamide | 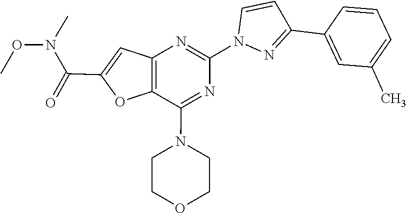 [SMILES: CON(C)C(=O)c1cc2nc(-n3ccc(-c4cccc(C)c4)n3)nc(N3CCOCC3)c2o1] |
| 231 | (4-morpholino-2-(3-(m-tolyl)-1H- pyrazol-1-yl)furo[3,2-d]pyrimidin-6- yl)(piperidin-1-yl)methanone | 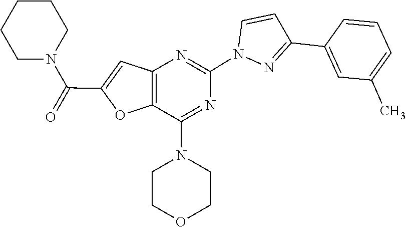 [SMILES: Cc1cccc(-c2ccn(-c3nc(N4CCOCC4)c4oc(C(=O)N5CCCCC5)cc4n3)n2)c1] |
| 232 | azetidin-1-yl(4-morpholino-2-(3-(m- tolyl)-1H-pyrazol-1-yl)furo[3,2- d]pyrimidin-6-yl)methanone | 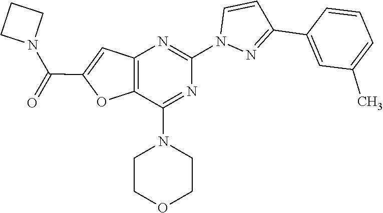 [SMILES: Cc1cccc(-c2ccn(-c3nc(N4CCOCC4)c4oc(C(=O)N5CCC5)cc4n3)n2)c1] |
| 233 | (4-morpholino-2-(3-(m-tolyl)-1H- pyrazol-1-yl)furo[3,2-d]pyrimidin-6- yl)(pyrrolidin-1-yl)methanone | 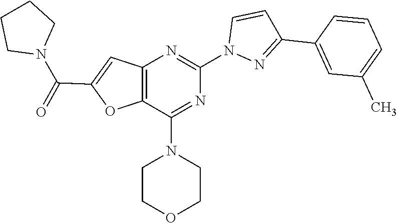 [SMILES: Cc1cccc(-c2ccn(-c3nc(N4CCOCC4)c4oc(C(=O)N5CCCC5)cc4n3)n2)c1] |
| 234 | N-ethyl-N-methyl-4-morpholino-2-(3- (m-tolyl)-1H-pyrazol-1-yl)furo[3,2- d]pyrimidine-6-carboxamide | 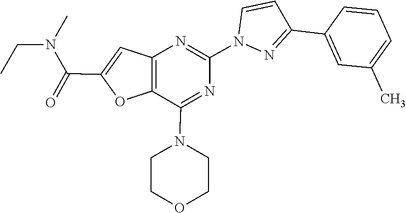 [SMILES: CCN(C)C(=O)c1cc2nc(-n3ccc(-c4cccc(C)c4)n3)nc(N3CCOCC3)c2o1] |
| 235 | N-cyclopropyl-N-methyl-4-morpholino- 2-(3-(m-tolyl)-1H-pyrazol-1-yl)furo[3,2- d]pyrimidine-6-carboxamide | 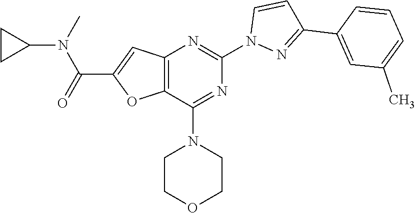 [SMILES: Cc1cccc(-c2ccn(-c3nc(N4CCOCC4)c4oc(C(=O)N(C)C5CC5)cc4n3)n2)c1] |
| 236 | N-(cyclopropylmethyl)-N-methyl-4- morpholino-2-(3-(m-tolyl)-1H-pyrazol- 1-yl)furo[3,2-d]pyrimidine-6- carboxamide | 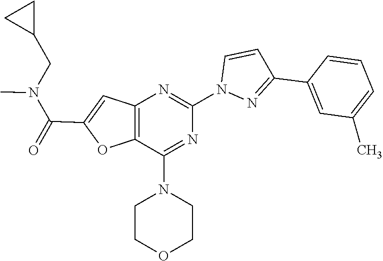 [SMILES: Cc1cccc(-c2ccn(-c3nc(N4CCOCC4)c4oc(C(=O)N(C)CC5CC5)cc4n3)n2)c1] |
| 237 | 4-morpholino-6-(pyrimidin-4-yl)-2-(4- (m-tolyl)-1H-pyrazol-1-yl)furo[3,2- d]pyrimidine | 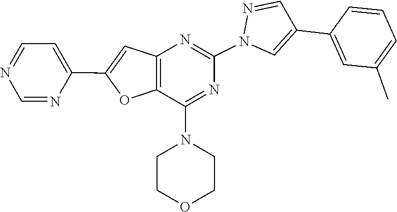 [SMILES: Cc1cccc(-c2cnn(-c3nc(N4CCOCC4)c4oc(-c5ccncn5)cc4n3)c2)c1] |
| 238 | 6-(3-methyl-1H-pyrazol-5-yl)-4- morpholino-2-(4-(m-tolyl)-1H-pyrazol- 1-yl)furo[3,2-d]pyrimidine | 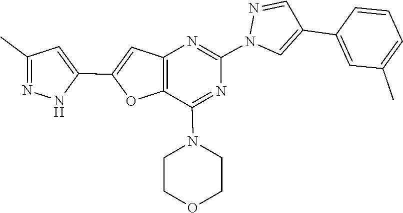 [SMILES: Cc1cccc(-c2cnn(-c3nc(N4CCOCC4)c4oc(-c5cc(C)n[nH]5)cc4n3)c2)c1] |
| 239 | N,N-dimethyl-2-(3-methyl-5-(4- morpholino-2-(4-(m-tolyl)-1H-pyrazol- 1-yl)furo[3,2-d]pyrimidin-6-yl)-1H- pyrazol-1-yl)ethan-1-amine | 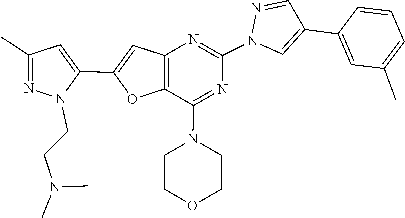 [SMILES: Cc1cccc(-c2cnn(-c3nc(N4CCOCC4)c4oc(-c5cc(C)nn5CCN(C)C)cc4n3)c2)c1] |
| 240 | N,N-dimethyl-2-(5-methyl-3-(4- morpholino-2-(4-(m-tolyl)-1H-pyrazol- 1-yl)furo[3,2-d]pyrimidin-6-yl)-1H- pyrazol-1-yl)ethan-1-amine | 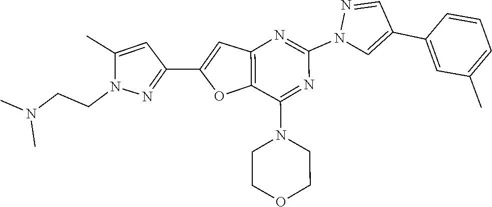 [SMILES: Cc1cccc(-c2cnn(-c3nc(N4CCOCC4)c4oc(-c5cc(C)n(CCN(C)C)n5)cc4n3)c2)c1] |
| 241 | N,N-dimethyl-1-(6-(4-morpholino-2-(4- (m-tolyl)-1H-pyrazol-1-yl)furo[3,2- d]pyrimidin-6-yl)pyridin-3- yl)methanamine | 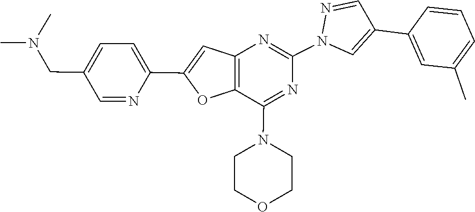 [SMILES: Cc1cccc(-c2cnn(-c3nc(N4CCOCC4)c4oc(-c5ccc(CN(C)C)cn5)cc4n3)c2)c1] |
| 242 | N-(2-(dimethylamino)ethyl)-6-(4- morpholino-2-(4-(m-tolyl)-1H-pyrazol- 1-yl)furo[3,2-d]pyrimidin-6- yl)nicotinamide | 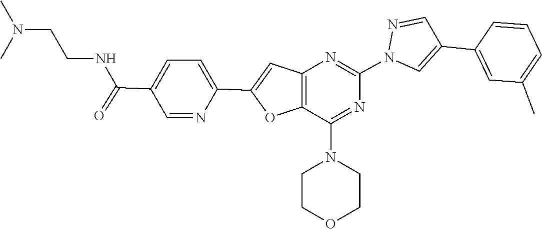 [SMILES: Cc1cccc(-c2cnn(-c3nc(N4CCOCC4)c4oc(-c5ccc(C(=O)NCCN(C)C)cn5)cc4n3)c2)c1] |
| 243 | N1,N1-dimethyl-N2-(6-(4-morpholino- 2-(4-(m-tolyl)-1H-pyrazol-1-yl)furo[3,2- d]pyrimidin-6-yl)pyridin-2-yl)ethane- 1,2-diamine | 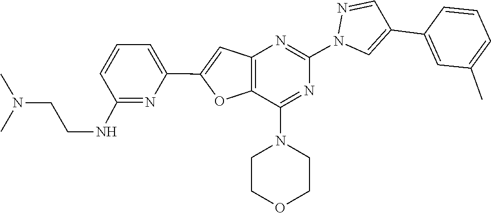 [SMILES: Cc1cccc(-c2cnn(-c3nc(N4CCOCC4)c4oc(-c5cccc(NCCN(C)C)n5)cc4n3)c2)c1] |
| 244 | \V-dimctliv1-2-(5-(4-inorplioliiio-2-(4- (m-tolyl)-l //-pyrazol-1-yl)furo[3,2- dlpyrimidin-6-yl)-1H-pyrazol-3- yl)ethan-1-amine | 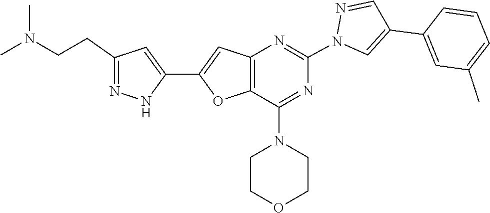 [SMILES: Cc1cccc(-c2cnn(-c3nc(N4CCOCC4)c4oc(-c5cc(CCN(C)C)n[nH]5)cc4n3)c2)c1] |
| 245 | N-ethyl-4-(3-oxomorpholino)-2-(4-(m- tolyl)-1H-pyrazol-1-yl)furo[3,2- d]pyrimidine-6-carboxamide | 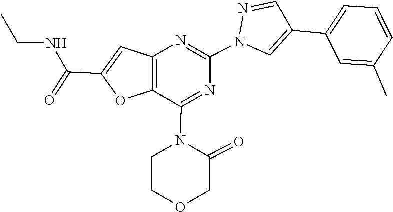 [SMILES: CCNC(=O)c1cc2nc(-n3cc(-c4cccc(C)c4)cn3)nc(N3CCOCC3=O)c2o1] |
| 246 | N-ethyl-4-(3-oxopiperazin-1-yl)-2-(4- (m-tolyl)-1H-pyrazol-1-yl)furo[3,2- d]pyrimidine-6-carboxamide | 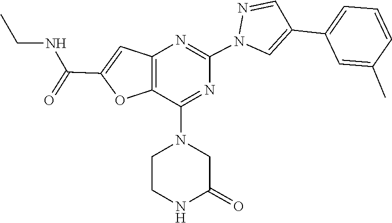 [SMILES: CCNC(=O)c1cc2nc(-n3cc(-c4cccc(C)c4)cn3)nc(N3CCNC(=O)C3)c2o1] |
| 247 | N-(1H-pyrazol-4-yl)-6-(pyridin-3-yl)-2- (4-(m-tolyl)-1H-pyrazol-1-yl)furo[3,2- d]pyrimidin-4-amine | 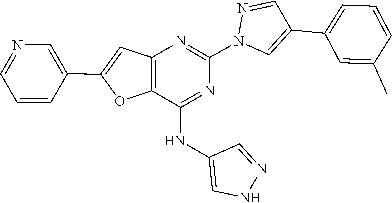 [SMILES: Cc1cccc(-c2cnn(-c3nc(Nc4cn[nH]c4)c4oc(-c5cccnc5)cc4n3)c2)c1] |
| 248 | N-(1H-pyrazol-3-yl)-6-(pyridin-3-yl)-2- (4-(m-tolyl)-1H-pyrazol-1-yl)furo[3,2- d]pyrimidin-4-amine | 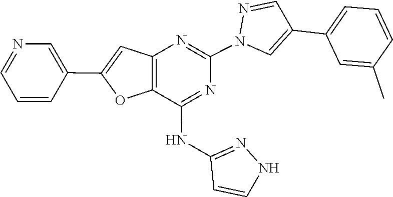 [SMILES: Cc1cccc(-c2cnn(-c3nc(Nc4cc[nH]n4)c4oc(-c5cccnc5)cc4n3)c2)c1] |
| 249 | 1-(6-(pyridin-3-yl)-2-(4-(m-tolyl)-1H- pyrazol-1-yl)furo[3,2-d]pyrimidin-4-yl)- 1H-pyrazol-4-amine | 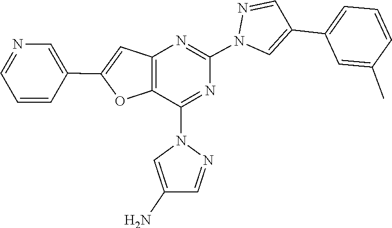 [SMILES: Cc1cccc(-c2cnn(-c3nc(-n4cc(N)cn4)c4oc(-c5cccnc5)cc4n3)c2)c1] |
| 250 | 1-(6-(pyridin-3-yl)-2-(4-(m-tolyl)-1H- pyrazol-1-yl)furo[3,2-d]pyrimidin-4-yl)- 1H-pyrazol-3-amine | 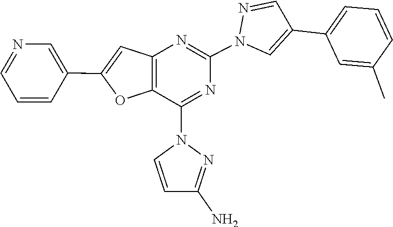 [SMILES: Cc1cccc(-c2cnn(-c3nc(-n4ccc(N)n4)c4oc(-c5cccnc5)cc4n3)c2)c1] |
| 251 | N-(1H-imidazol-4-yl)-6-(pyridin-3-yl)- 2-(4-(m-tolyl)-1H-pyrazol-1-yl)furo[3,2- d]pyrimidin-4-amine | 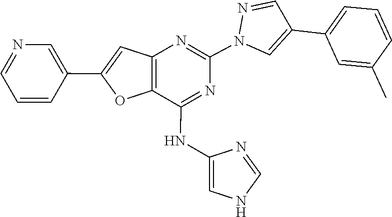 [SMILES: Cc1cccc(-c2cnn(-c3nc(Nc4c[nH]cn4)c4oc(-c5cccnc5)cc4n3)c2)c1] |
| 252 | 1-(6-(pyridin-3-yl)-2-(4-(m-tolyl)-1H- pyrazol-1-yl)furo[3,2-d]pyrimidin-4-yl)- 1H-imidazol-4-amine | 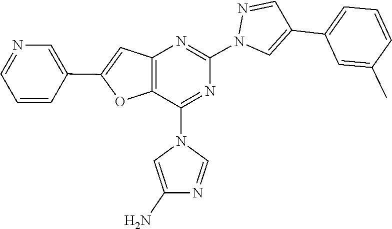 [SMILES: Cc1cccc(-c2cnn(-c3nc(-n4cnc(N)c4)c4oc(-c5cccnc5)cc4n3)c2)c1] |
| 253 | N,N-dimethyl-2-(1-methyl-3-(4- morpholino-2-(4-phenyl-1H-pyrazol-1- yl)furo[3,2-d]pyrimidin-6-yl)-1H- pyrazol-5-yl)ethan-1-amine | 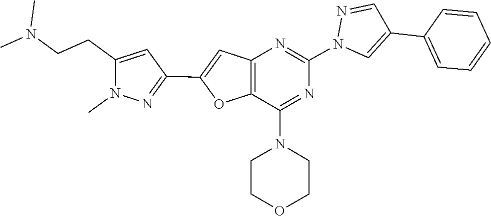 [SMILES: CN(C)CCc1cc(-c2cc3nc(-n4cc(-c5ccccc5)cn4)nc(N4CCOCC4)c3o2)nn1C] |
| 254 | N,N-dimethyl-2-(1-methyl-3-(4- morpholino-2-(3-phenyl-1H-pyrazol-1- yl)furo[3,2-d]pyrimidin-6-yl)-1H- pyrazol-5-yl)ethan-1-amine | 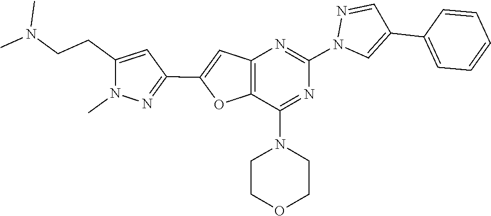 [SMILES: CN(C)CCc1cc(-c2cc3nc(-n4cc(-c5ccccc5)cn4)nc(N4CCOCC4)c3o2)nn1C] |
| 255 | 6-(1H-imidazol-2-yl)-4-morpholino-2- (3-(m-tolyl)-1H-pyrazol-1-yl)furo[3,2- d]pyrimidine | 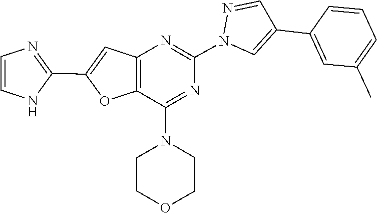 [SMILES: Cc1cccc(-c2cnn(-c3nc(N4CCOCC4)c4oc(-c5ncc[nH]5)cc4n3)c2)c1] |
| 256 | 6-(1H-imidazol-2-yl)-4-morpholino-2- (3-phenyl-1H-pyrazol-1-yl)furo[3,2- d]pyrimidine | 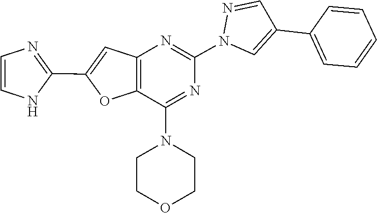 [SMILES: c1ccc(-c2cnn(-c3nc(N4CCOCC4)c4oc(-c5ncc[nH]5)cc4n3)c2)cc1] |
| 257 | N,N-dimethyl-2-(1-methyl-3-(4- morpholino-2-(4-(m-tolyl)-1H-pyrazol- 1-yl)furo[3,2-d]pyrimidin-6-yl)-1H- pyrazol-5-yl)ethan-1-amine | 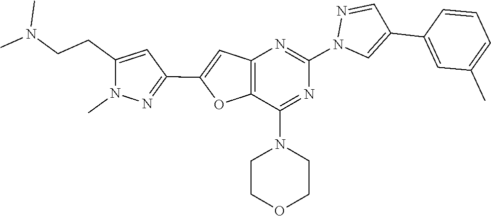 [SMILES: Cc1cccc(-c2cnn(-c3nc(N4CCOCC4)c4oc(-c5cc(CCN(C)C)n(C)n5)cc4n3)c2)c1] |

and pharmaceutically acceptable salts thereof.

### Methods of Treating, Administration, and Pharmaceutical Compositions

In general, the compounds of this disclosure will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. In one embodiment, the dosage level will be about 0.1 to about 250 mg/kg per day. In another embodiment the dosage level will be about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day. For oral administration, the compositions may be provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this disclosure, i.e., the active ingredient, will depend upon numerous factors such as the severity of the disease to be treated, the age and relative health of the subject, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

Pharmaceutical compositions can be formulated using one or more pharmaceutically acceptable carriers comprising excipients and auxiliaries. The formulation can be modified depending upon the route of administration chosen. The pharmaceutical compositions can also include the compounds described herein in a free base form or a pharmaceutically acceptable salt or prodrug form.

Methods for formulation of the pharmaceutical compositions can include formulating any of the compounds described herein with one or more inert, pharmaceutically acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically acceptable additives. Alternatively, the compositions described herein can be lyophilized or in powder form for re-constitution with a suitable vehicle, e.g., sterile pyrogen-free water, before use. The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The pharmaceutical compositions and formulations can be sterilized. Sterilization can be accomplished by filtration through sterile filtration.

The pharmaceutical compositions described herein can be formulated for administration as an injection. Non-limiting examples of formulations for injection can include a sterile suspension, solution, or emulsion in oily or aqueous vehicles. Suitable oily vehicles can include, but are not limited to, lipophilic solvents or vehicles such as fatty oils, synthetic fatty acid esters, or liposomes. Aqueous injection suspensions can contain substances which increase the viscosity of the suspension. The suspension can also contain suitable stabilizers. Injections can be formulated for bolus injection or continuous infusion.

For parenteral administration, the compounds can be formulated in a unit dosage injectable form (e.g., solution, suspension, emulsion) in association with a pharmaceutically acceptable parenteral vehicle. Such vehicles can be inherently nontoxic, and non-therapeutic. A vehicle can be water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release matrices can include polyesters, hydrogels (e.g., poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations of the compositions described herein can be prepared for storage by mixing a compound with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Compounds of the present disclosure may be used in methods of treating in combination with one or more other combination agents (e.g., one, two, or three other drugs) that are used in the prevention, treatment, control, amelioration, or reduction of risk of the diseases or conditions for which compounds of the present disclosure are useful. In some embodiments, the combination of the drugs together are safer or more effective than either drug alone. In some embodiments the compound disclosed herein and the one or more combination agents have complementary activities that do not adversely affect each other. Such molecules can be present in combination in amounts that are effective for the purpose intended. Such other drug(s) may be administered, by a route and in an amount commonly used therefore, contemporaneously or sequentially with a compound of the present disclosure. When a compound of the present disclosure is used contemporaneously with one or more other drugs, in some embodiments, the agents are administered together in a single pharmaceutical composition in unit dosage form. Accordingly, the pharmaceutical compositions of the present disclosure also include those that contain one or more other active ingredients, in addition to a compound of the present disclosure. The weight ratio of the compound of the present disclosure to the second active agent may be varied and will depend upon the effective dose of each ingredient. Generally, an effective dose of each will be used. In some embodiments, combination therapy includes therapies in which the compound of the present disclosure and one or more other drugs are administered separately, and in some cases, the two or more agents are administered on different, overlapping schedules. It is also contemplated that when used in combination with one or more other active ingredients, the compounds of the present disclosure and the other active ingredients may be used in lower doses than when each is used singly. For example, the combination agent is an anticancer agent, such as an alkylating agent, a corticosteroid, a platinum drug, a purine analog, an anti-metabolite, or particular agents such as cyclophosphamide, chlorambucil, bendamustine, prednisone, dexamethasone, carboplatin, cisplatin, cladribine, fludarabine, capecitabine, gemcitabine, methotrexate, pralatrexate, bleomycin, doxorubicin, vincristine, or rituximab. In some embodiments, the combination agent is a drug for reduction of symptoms of ALS. In some embodiments, the combination agent is selected from an NAD supplement (such as nicotinamide riboside, offered under the trade names Basis® or Tru Niagen®), vitamin B₁₂ (oral or injection), glycopyrrolate, atropine, scopolamine, baclofen, tizanidine, mexiletine, an SSRI, a benzodiazepine, Neudexta, riluzole, and edaravone, and combinations thereof.

The compounds, pharmaceutical compositions, and methods of the present disclosure can be useful for treating a subject such as, but not limited to, a mammal, a human, a non-human mammal, a domesticated animal (e.g., laboratory animals, household pets, or livestock), a non-domesticated animal (e.g., wildlife), a dog, a cat, a rodent, a mouse, a hamster, a cow, a bird, a chicken, a fish, a pig, a horse, a goat, a sheep, or a rabbit. In preferred embodiments, compounds, pharmaceutical compositions, and methods of the present disclosure are used for treating a human.

The compounds, pharmaceutical compositions, and methods described herein can be useful as a therapeutic, for example a treatment that can be administered to a subject in need thereof. A therapeutic effect can be obtained in a subject by reduction, suppression, remission, or eradication of a disease state, including, but not limited to, a symptom thereof. A therapeutic effect in a subject having a disease or condition, or pre-disposed to have or is beginning to have the disease or condition, can be obtained by a reduction, a suppression, a prevention, a remission, or an eradication of the condition or disease, or pre-condition or pre-disease state.

In practicing the methods described herein, therapeutically effective amounts of the compounds or pharmaceutical compositions described herein can be administered to a subject in need thereof, often for treating and/or preventing a condition or progression thereof. A pharmaceutical composition can affect the physiology of the subject, such as the immune system, inflammatory response, or other physiologic affect. A therapeutically effective amount can vary widely depending on the severity of the disease, the age and relative health of the subject, the potency of the compounds used, and other factors.

Treat and/or treating can refer to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treat can be used herein to refer to a method that results in some level of treatment or amelioration of the disease or condition and can contemplate a range of results directed to that end, including but not restricted to prevention of the condition entirely.

Prevent, preventing, and the like can refer to the prevention of the disease or condition in the patient. For example, if an individual at risk of contracting a disease is treated with the methods of the present disclosure and does not later contract the disease, then the disease has been prevented, at least over a period of time, in that individual.

A therapeutically effective amount can be the amount of a compound or pharmaceutical composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques.

The compounds or pharmaceutical compositions described herein that can be used in therapy can be formulated and dosages established in a fashion consistent with good medical practice taking into account the disorder to be treated, the condition of the individual patient, the site of delivery of the compound or pharmaceutical composition, the method of administration and other factors known to practitioners. The compounds or pharmaceutical compositions can be prepared according to the description of preparation described herein.

One of ordinary skill in the art would understand that the amount, duration, and frequency of administration of a pharmaceutical composition or compound described herein to a subject in need thereof depends on several factors including, for example but not limited to, the health of the subject, the specific disease or condition of the patient, the grade or level of a specific disease or condition of the patient, the additional therapeutics the subject is being or has been administered, and the like.

The methods, compounds, and pharmaceutical compositions described herein can be for administration to a subject in need thereof. Often, administration of the compounds or pharmaceutical compositions can include routes of administration, non-limiting examples of administration routes include intravenous, intraarterial, subcutaneous, subdural, intramuscular, intracranial, intrasternal, intratumoral, or intraperitoneally. Additionally, a pharmaceutical composition or compound can be administered to a subject by additional routes of administration, for example, by inhalation, oral, dermal, intranasal, or intrathecal administration.

Pharmaceutical compositions or compounds of the present disclosure can be administered to a subject in need thereof in a first administration, and in one or more additional administrations. The one or more additional administrations can be administered to the subject in need thereof minutes, hours, days, weeks, or months following the first administration. Any one of the additional administrations can be administered to the subject in need thereof less than 21 days, or less than 14 days, less than 10 days, less than 7 days, less than 4 days or less than 1 day after the first administration. The one or more administrations can occur more than once per day, more than once per week, or more than once per month. The compounds or pharmaceutical compositions can be administered to the subject in need thereof in cycles of 21 days, 14 days, 10 days, 7 days, 4 days, or daily over a period of one to seven days.

The compounds, pharmaceutical compositions, and methods provided herein can be useful for the treatment of a plurality of diseases or conditions or preventing a disease or a condition in a subject, or other therapeutic applications for subjects in need thereof. In one aspect, the disclosure relates to a method of inhibiting PIKfyve and/or a PI3 kinase in a subject in need thereof comprising administering to the subject an effective amount of a compound. In one aspect, the disclosure relates to a method for treating a neurological disease mediated by PIKfyve activity and/or a PI3 kinase activity in a subject in need thereof, comprising administering an effective amount of a compound or a pharmaceutical composition as described herein to the subject. In some aspects, the disease is a neurological disease. In some embodiments, the disease is associated with a FIG. 4 deficiency. In some embodiments is a method for treating a subject with a neurological disease or disorder associated with PIKfyve and/or PI3 kinase activity, comprising administering to the subject an effective amount of a compound or pharmaceutical composition as described herein.

In some embodiments, the neurological disease is amyotrophic lateral sclerosis (ALS), primary lateral sclerosis (PLS), Charcot-Marie-Tooth (CMT; including type 4J (CMT4J)), and Yunis-Varon syndrome, autophagy, polymicrogyria (including polymicrogyria with seizures), temporo-occipital polymicrogyria, Pick's disease, Parkinson's disease, Parkinson's disease with Lewy bodies, dementia with Lewy bodies, Lewy body disease, frontotemporal dementia, diseases of neuronal nuclear inclusions of polyglutamine and intranuclear inclusion bodies, disease of Marinesco and Hirano bodies, tauopathy, Alzheimer's disease, neurodegeneration, spongiform neurodegeneration, peripheral neuropathy, leukoencephalopathy, inclusion body disease, progressive supranuclear palsy, corticobasal syndrome, chronic traumatic encephalopathy, traumatic brain injury (TBI), cerebral ischemia, Guillain-Barre Syndrome, chronic inflammatory demyelinating polyneuropathy, multiple sclerosis, a lysosomal storage disease, Fabry's disorder, Gaucher's disorder, Niemann Pick C disease, Tay-Sachs disease, and Mucolipidosis type IV, neuropathy, Huntington's disease, a psychiatric disorder, ADHD, schizophrenia, a mood disorder, major depressive disorder, depression, bipolar disorder I, or bipolar disorder II.

In some embodiments, the neurological disease is ALS, FTD, Alzheimer's disease, Parkinson's disease, Huntington's disease, or CMT. In some embodiments, the neurological disease is ALS.

In some embodiments, the neurological disease is a tauopathy such as Alzheimer's disease, progressive supranuclear palsy, corticobasal syndrome, frontotemporal dementia, or chronic traumatic encephalopathy.

In some embodiments, the neurological disease is a lysosomal storage disease such as Fabry's disorder, Gaucher's disorder, Niemann Pick C disease, Tay-Sachs disease, or Mucolipidosis type IV.

In some embodiments, the neurological disease is a psychiatric disorder such as ADHD, schizophrenia, or mood disorders such as major depressive disorder, depression, bipolar disorder I, or bipolar disorder II.

In some aspects is a method of treating a disease mediated by PI3K activity in a subject in need thereof, comprising administering an effective amount of a compound or a pharmaceutical composition as described herein to the subject. In some embodiments, the PI3K is a PI3K isoform, such as PI3Kα, β, δ, and/or γ. In some embodiments, the disease is a neurological disease.

The disclosure further provides any compounds disclosed herein for use in a method of treatment of the human or animal body by therapy. Therapy may be by any mechanism disclosed herein, such as inhibiting, reducing, or reducing progression of the diseases disclosed herein. The disclosure further provides any compound disclosed herein for prevention or treatment of any condition disclosed herein. The disclosure also provides any compound or pharmaceutical composition thereof disclosed herein for obtaining any clinical outcome disclosed herein for any condition disclosed herein. The disclosure also provides use of any compound disclosed herein in the manufacture of a medicament for preventing or treating any disease or condition disclosed herein.

### EXAMPLES

The following preparations of compounds of Formula (I) and intermediates are given to enable those skilled in the art to more clearly understand and to practice the present disclosure. They should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Rcagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., or from about 0° C. to about 125° C. or at about room (or ambient) temperature, e.g., about 20° C.

Compounds of Formula (I) and subformulae and species described herein, including those where the substituent groups as defined herein, can be prepared as illustrated and described below.

Unless otherwise noted, all reagents were used without further purification. ¹H NMR spectra were obtained in CDCl₃, DMSO-d₆, or CD₃OD at room temperature on a Bruker 300 MHz instrument. When more than one conformer was detected, the chemical shifts for the most abundant one is reported. Chemical shifts of ¹H NMR spectra were recorded in parts per million (ppm) on the δ scale from an internal standard of residual solvent. Splitting patterns are designed as s, singlet; d, doublet; t, triplet; q, quartet; m, multiplet; br, broad. LC-MS conditions were as described below:

LCMS Column: Agilent Zorbax XDB C18 4.6×50 mm, 3.5 μm
a. Mobile phase: Solvent A: Water (with 0.1% formic acid); Solvent B: MeOH
b. Flow rate: 1.0 mL/min,
c. Run time: 2 min gradient (20%-90% B), then 3 min @90% B,
d. Temperature: 30° C.

HPLC Column: Agilent SB-C18 4.6×150 mm, 3.5 μm
a. Mobile phase: Solvent A: water (with 0.02% TFA); Solvent B: MeOH
b. Flow rate: 1.0 mL/min,
c. Run time: 0.5 min @10% B, 9.5 min gradient (10%-90% B), then 10 min @90% B,
d. Temperature: 30° C.

Preparative LC Column: Phenomenex Luna 5 u 100 A, 21.2×250 mm, 5 μm
a. Mobile phase: Solvent A: Water
   i. Solvent B: MeOH
b. Flow rate: 10 mL/min,
c. Run time: 1 min @20% B, 30 min gradient (20%-80% B), then 10 min @90% B,
d. Temperature: Ambient

The following abbreviations are used in the text: PE=petroleum ether, EA or AcOH=acetic acid, EtOAc=ethyl acetate, DMSO=dimethyl sulfoxide, DMF=N, N-dimethylacetamide, MeOH=methanol, i-PrOH=isopropyl alcohol, MTBE=Methyl tert-butyl ether, DCM=dichloromethane, Et₃N or TEA=triethylamine, DIPEA=Diisopropylethylamine, DIEA=N,N-Diisopropylethylamine, TFA=trifluoroacetic acid, TLC=thin layer chromatography, (BPin)₂=Bis(pinacolato)diboron, HFIP=1,1,1,3,3,3-hexafluoropropan-2-ol, DIBAL-H=Diisobutylaluminum hydride, Mel=Iodomethane, n-Hex=n-Hexane, DCE=1,2-Dichloroethane, TBSCl=tert-Butyldimethylsilyl chloride, Tf₂O=Trifluoromethanesulfonic anhydride, n-BuLi=n-Butyllithium, DMAP=4-Dimethylaminopyridine, KOAc=Potassium acetate, NaOAc=Sodium acetate, TFAA=Trifluoroacetic anhydride, m-CPBA=meta-Chloroperoxybenzoic acid, DME=1,2-Dimethoxyethane, PS-TPP=polymer supported triphenylphosphine, MSA=methanesulfonic acid, SEMCI=2-(Trimethylsilyl)ethoxymethyl chloride, Et₂O=diethylether, THF=tetrahydrofuran, NIS=N-Iodosuccinimide, LDA=Lithium diisopropylamide, EDCl=1-Ethyl-3-(3-dimethylaminopropyl)carbodiimide, TMSCF₃=Trifluoromethyltrimethylsilane, Xantphos=4,5-Bis(diphenylphosphino)-9,9-dimethylxanthene, h and hr=hour, rt=room temperature, Ph=phenyl, dppf=1,1′-Bis(diphenylphosphino)-ferrocene, dba=dibenzylideneacetone,

### Example 1: Compound 1 Using General Synthetic Route 1

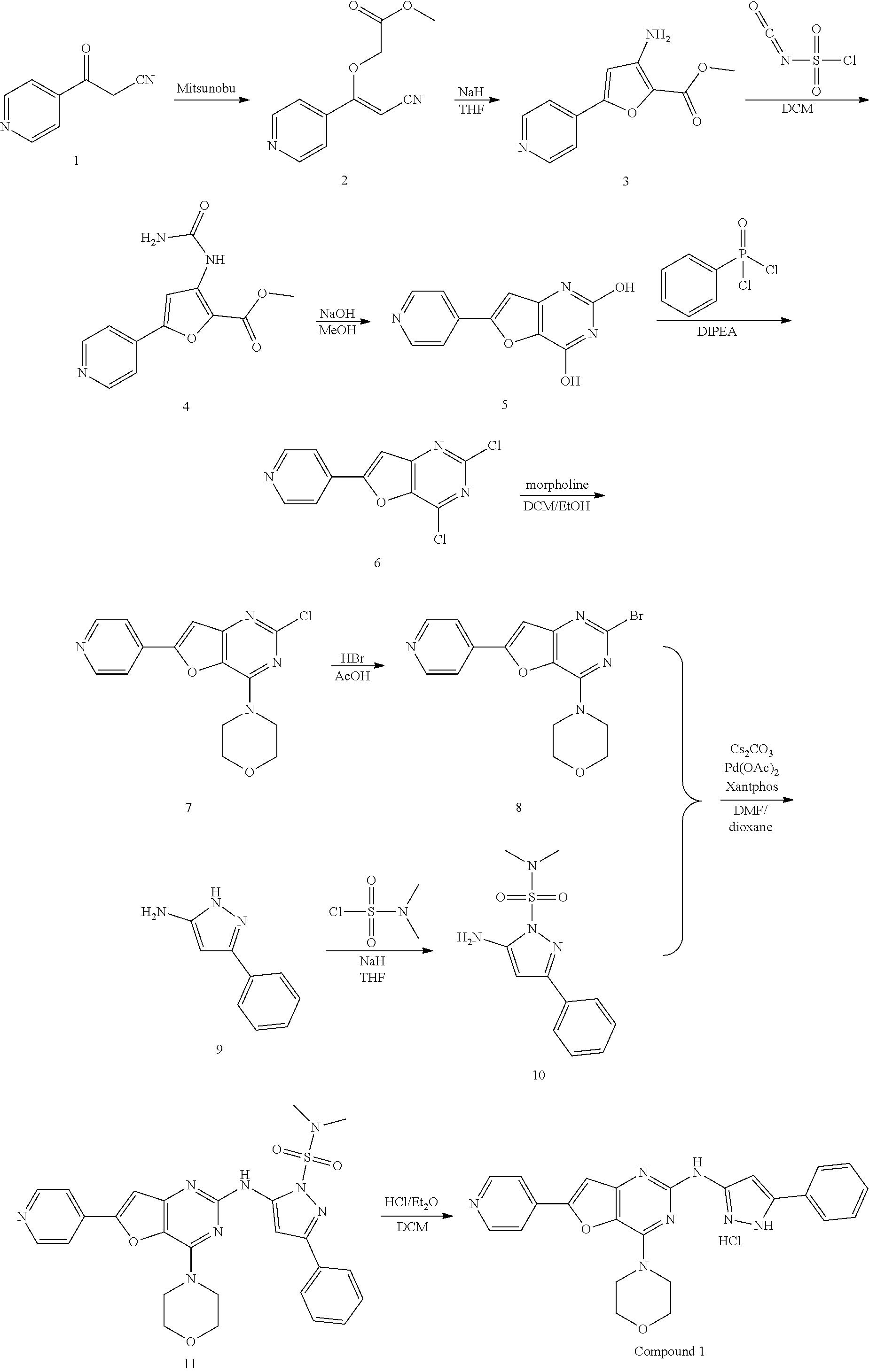

### 1.1) Synthesis of (Z)-methyl 2-((2-cyano-1-(pyridin-4-yl)vinyl)oxy)acetate

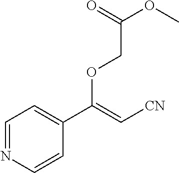 [SMILES: COC(=O)CO/C(=C\C#N)c1ccncc1]

To a solution of triphenylphosphine (3.5 g, 13.4 mmol) in dry THF was added diethyl azodicarboxylate (DEAD) (2.3 g, 13.4 mmol), 3-oxo-3-(pyridin-4-yl)propanenitrile (1.5 g, 10.3 mmol) and methyl 2-hydroxyacetate (1.2 g, 13.4 mmol) under N₂. The reaction was stirred at ambient temperature overnight. The reaction mixture was concentrated directly and purified by silica gel column chromatography with a gradient elution of 1% MeOH/DCM to 2% MeOH/DCM to provide 4.4 g of impure (Z)-methyl2-((2-cyano-1-(pyridin-4-yl)vinyl)oxy)acetate containing triphenylphosphine oxide as a yellow solid. LC-MS (ESI+): m/z 219 (MH⁺).

### 1.2) Synthesis of methyl 3-amino-5-(pyridin-4-yl)furan-2-carboxylate

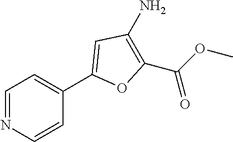 [SMILES: COC(=O)c1oc(-c2ccncc2)cc1N]

To a solution of impure (Z)-methyl2-((2-cyano-1-(pyridin-4-yl)vinyl)oxy)acetate (4.6 g, 21.1 mmol) in dry THE at 0° C. was added NaH (1.5 g, 31.6 mmol). The reaction was warmed to room temperature and stirred for 2 h. The reaction mixture was quenched with a saturated NH₄Cl solution and the pH was adjusted to 3 using 2 N HCl aqueous solutions. The aqueous solution was extracted with ethyl acetate (3×50 mL) to remove some impurities. To the remaining aqueous solution was added a saturated Na₂CO₃ solution to adjust the pH to 11. The resulting aqueous solution was extracted with DCM/MeOH (10/1, 3×60 mL). The combined organic phase was dried over anhydrous Na₂SO₄, filtrated and concentrated to provide 1.35 g of crude methyl 3-amino-5-(pyridin-4-yl)furan-2-carboxylate as an oil. The crude product was used directly for the next step without further purification. LC-MS (ESI+): m/z 219 (MH⁺). ¹HNMR (300 MHz, CDCl₃) δ 8.66 (dd, J=4.5, 1.5 Hz, 2H), 7.58 (dd, J=4.8, 1.2 Hz, 2H), 6.58 (s, 1H), 4.67 (brs, 2H), 3.93 (s, 3H).

### 1.3) Synthesis of methyl 5-(pyridin-4-yl)-3-ureidofuran-2-carboxylate

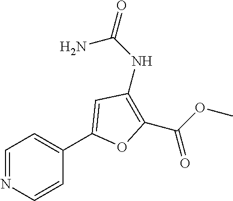 [SMILES: COC(=O)c1oc(-c2ccncc2)cc1NC(N)=O]

To a solution of methyl 3-amino-5-(pyridin-4-yl)furan-2-carboxylate (1.94 g, 8.9 mmol) in DCM (40 mL) at −78° C. under N₂ was added chlorosulfonyl isocyante (3.79 g, 26.7 mmol) dropwise. After addition, the reaction was warmed to room temperature and stirred for 1 h. After removal of DCM by evaporation, the resulting residue was treated with 6 N HCl (10 mL) aqueous solutions. The mixture was heated to reflux for 30 min. The completion of the reaction was monitored by thin layer chromatography (TLC). The reaction was cooled to room temperature and the pH was adjusted to 9 using a saturated NaHCO₃ solution. A large amount of solid was precipitated. After filtration, the filter cake was washed with water and dried to provide 2.7 g of crude methyl5-(pyridin-4-yl)-3-ureidofuran-2-carboxylate as a yellow solid. LC-MS (ESI+): m/z 262 (MH⁺). ¹HNMR (300 MHz, CD₃OD) δ 8.61 (dd, J=4.8, 1.5 Hz, 2H), 7.89 (s, 1H), 7.78 (dd, J=5.1, 1.5 Hz, 2H), 3.95 (s, 3H).

### 1.4) Synthesis of 6-(pyridin-4-yl)furo[3,2-d]pyrimidine-2,4-diol

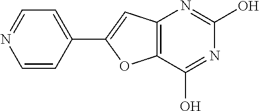 [SMILES: Oc1nc(O)c2oc(-c3ccncc3)cc2n1]

To a solution of crude methyl 5-(pyridin-4-yl)-3-ureidofuran-2-carboxylate (2.7 g, 10.3 mmol) in MeOH (40 mL) was added 1.5 N NaOH (15 mL). The reaction was heated to reflux for 1.5 h. The completion of the reaction was monitored by TLC. The solvent MeOH was removed by evaporation. To the resulting residue were added 6 N HCl solutions until the pH was adjusted to 2. A large amount of solid was precipitated. After filtration, the filter cake was washed with water and dried to provide crude 2.1 g of 6-(pyridin-4-yl)furo[3,2-d]pyrimidine-2,4-diol as a yellow solid. LC-MS (ESI+): m/z 230 (MH⁺). ¹HNMR (300 MHz, DMSO-d₆) δ 11.61 (s, 1H), 11.37 (s, 1H), 8.89 (d, J=6.6 Hz, 2H), 8.24 (d, J=6.3 Hz, 2H), 7.63 (s, 1H).

### 1.5) Synthesis of 2,4-dichloro-6-(pyridin-4-yl)furo[3,2-d]pyrimidine

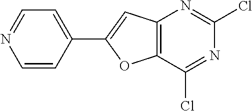 [SMILES: Clc1nc(Cl)c2oc(-c3ccncc3)cc2n1]

To a solution of 6-(pyridin-4-yl)furo[3,2-d]pyrimidine-2,4-diol (1.5 g, 6.54 mmol) in phenylphosphonic dichloride (30 mL) was added DIPEA (8.43 g, 65.4 mmol). The reaction was heated to 120° C. overnight. After the reaction mixture was cooled to room temperature, a saturated NaHCO₃ solution was added to adjust the pH to 8. The aqueous solution was extracted with EtOAc (3×50 mL). The combined organic phase was dried over anhydrous Na₂SO₄, filtrated and concentrated. The resulting residue was purified by silica gel column chromatography with a gradient elution of 50% EtOAc/PE to 75% EtOAc/PE to provide 2,4-dichloro-6-(pyridin-4-yl)furo[3,2-d]pyrimidine (1.6 g, 6.9 mmol) as a yellow solid. LC-MS (ESI+): m/z 266/268 (MH⁺). ¹HNMR (300 MHz, CDCl₃) δ 8.85 (dd, J=4.8, 1.5 Hz, 2H), 7.82 (dd, J=4.5, 1.5 Hz, 2H), 7.38 (s, 1H).

### 1.6) Synthesis of 2-chloro-4-morpholino-6-(pyridin-4-yl)furo[3,2-d]pyrimidine

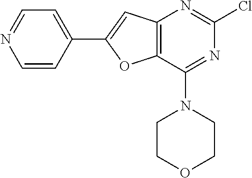 [SMILES: Clc1nc(N2CCOCC2)c2oc(-c3ccncc3)cc2n1]

To a solution of 2,4-dichloro-6-(pyridin-4-yl)furo[3,2-d]pyrimidine (1.6 g, 6.9 mmol) in DCM/EtOH (1/3, 120 mL) was added morpholine (0.91 g, 10.5 mmol) and K₂CO₃ (1.91 g, 14 mmol). The reaction was stirred at room temperature for 2 h. The reaction mixture was concentrated directly and purified by silica gel column chromatography with a gradient elution of 2% MeOH/DCM to 3% MeOH/DCM to provide 2-chloro-4-morpholino-6-(pyridin-4-yl) furo[3,2-d]pyrimidine (770 mg, 2.43 mmol) as a yellow solid. LC-MS (ESI+): m/z 317/319 (MH⁺). ¹HNMR (300 MHz, DMSO-d₆) δ 8.76 (d, J=6.0 Hz, 2H), 7.97 (d, J=6.0 Hz, 2H), 7.82 (s, 1H), 4.06-3.97 (m, 4H), 3.82-3.75 (m, 4H).

### 1.6) Synthesis of 2-bromo-4-morpholino-6-(pyridin-4-yl)furo[3,2-d]pyrimidine

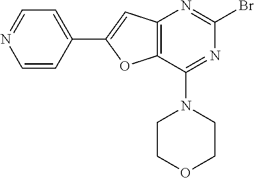 [SMILES: Brc1nc(N2CCOCC2)c2oc(-c3ccncc3)cc2n1]

A solution of 2,4-dichloro-6-(pyridin-4-yl)furo[3,2-d]pyrimidine (770 mg, 2.43 mmol) in HBr/AcOH (33 wt. % in Acetic acid, 10 mL) was heated to refluxed for 3.5 h. The completion of the reaction was monitored by LC-MS. The reaction mixture was quenched with a saturated NaHCO₃ solution and the pH was adjusted to 8. The aqueous solution was extracted with DCM/MeOH (15/1, 3×50 mL). The combined organic phase was dried over anhydrous Na₂SO₄, filtrated and concentrated under reduced pressure to provide 740 mg of crude 2-bromo-4-morpholino-6-(pyridin-4-yl)furo[3,2-d]pyrimidine as a brown solid. The crude product was used directly for the next step without further purification. LC-MS (ESI+): m/z 362/364 (MH⁺). ¹HNMR (300 MHz, DMSO-d₆) δ 8.76 (d, J=4.5 Hz, 2H), 7.98 (d, J=4.5 Hz, 2H), 7.82 (s, 1H), 4.02-3.95 (m, 4H), 3.83-3.76 (m, 4H).

### 1.7) Synthesis of 5-amino-N,N-dimethyl-3-phenyl-1H-pyrazole-1-sulfonamide

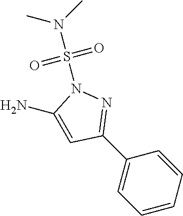 [SMILES: CN(C)S(=O)(=O)n1nc(-c2ccccc2)cc1N]

To a solution of 3-phenyl-1H-pyrazol-5-amine (300 mg, 1.88 mmol) in THF (5 mL) at 0° C. was added NaH (100 mg, 2.82 mmol). After stirring at 0° C. for 1 h, to the solution was added dimethylsulfamoyl chloride (315 mg, 2.20 mmol). The completion of the reaction was monitored by TLC. The reaction mixture was quenched with a saturated NH₄Cl solution. The aqueous solution was extracted with ethyl acetate (3×50 mL). The combined organic phase was dried over anhydrous Na₂SO₄, filtrated and concentrated under reduced pressure. The resulting residue was purified by silica gel column chromatography with a gradient elution of 20% EtOAc/PE to 33% EtOAc/PE to provide 5-amino-N,N-dimethyl-3-phenyl-1H-pyrazole-1-sulfonamide (300 mg, 1.13 mmol). LC-MS: m/z 267 (MH⁺). ¹HNMR (300 MHz, CDCl₃) δ 7.79-7.76 (m, 2H), 7.42-7.35 (m, 3H), 5.75 (s, 1H), 4.84 (s, 2H), 3.03 (s, 6H).

### 1.8) Synthesis of N,N-dimethyl-5-((4-morpholino-6-(pyridin-4-yl)furo[3,2-d]pyrimidin-2-yl)amino)-3-phenyl-1H-pyrazole-1-sulfonamide

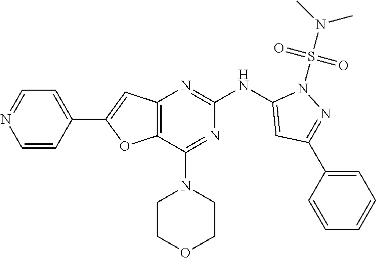 [SMILES: CN(C)S(=O)(=O)n1nc(-c2ccccc2)cc1Nc1nc(N2CCOCC2)c2oc(-c3ccncc3)cc2n1]

A suspension of 2-bromo-4-morpholino-6-(pyridin-4-yl)furo[3,2-d]pyrimidine (500 mg, 1.4 mmol), 3-amino-N,N-dimethyl-5-phenyl-1H-pyrazole-1-sulfonamide (554 mg, 2.1 mmol), Cs₂CO₃ (906 mg, 2.8 mmol), Pd(OAc)₂ (30 mg, 0.1 mmol) and Xantphos (80 mg, 0.1 mmol) in DMF/1,4-dioxane (1/7, 16 mL) was heated to 100° C. for 40 min under microwave conditions. The reaction mixture was concentrated directly and purified by silica gel column chromatography with a gradient elution of 1% MeOH/DCM to 2% MeOH/DCM to provide N,N-dimethyl-5-((4-morpholino-6-(pyridin-4-yl)furo[3,2-d]pyrimidin-2-yl)amino)-3-phenyl-1H-pyrazole-1-sulfonamide (140 mg, 0.26 mmol) as a white solid. LC-MS (ESI+): m/z 547 (MH⁺). ¹HNMR (300 MHz, CDCl₃) δ 8.76-8.71 (m, 3H), 7.91 (d, J=6.9 Hz, 2H), 7.64 (d, J=5.7 Hz, 2H), 7.47-7.39 (m, 3H), 7.28-7.22 (m, 2H), 4.14-4.08 (m, 4H), 3.94-3.87 (m, 4H), 3.07 (s, 6H).

### 1.9) Synthesis of 4-morpholino-N-(3-phenyl-1H-pyrazol-5-yl)-6-(pyridin-4-yl)furo[3,2-d]pyrimidin-2-amine hydrochloride

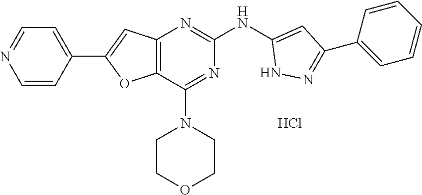 [SMILES: c1ccc(-c2cc(Nc3nc(N4CCOCC4)c4oc(-c5ccncc5)cc4n3)[nH]n2)cc1 ; Cl]

To a solution of N,N-dimethyl-5-((4-morpholino-6-(pyridin-4-yl)furo[3,2-d]pyrimidin-2-yl)amino)-3-phenyl-1H-pyrazole-1-sulfonamide (140 mg, 0.26 mmol) in DCM (4 mL) was added HCl/Et₂O (2 mL). The reaction mixture was stirred at room temperature for 2 h. After concentration and slurry in MeOH/Et₂O (1/20, 2 mL), 4-morpholino-N-(3-phenyl-1H-pyrazol-5-yl)-6-(pyridin-4-yl)furo[3,2-d] pyrimidin-2-amine hydrochloride (Compound 1, 112 mg, 0.22 mmol) was obtained as a yellow solid. LC-MS (ESI+): m/z 440 (MH⁺). ¹HNMR (300 MHz, CD₃OD) δ 8.97 (d, J=6.6 Hz, 2H), 8.55 (d, J=6.6 Hz, 2H), 8.05 (s, 1H), 7.37 (d, J=6.9 Hz, 2H), 7.66-7.40 (m, 3H), 6.44 (s, 1H), 4.35-4.27 (m, 4H), 4.01-3.92 (m, 4H).

### Example 2: Compound 10 Using General Synthetic Route 2

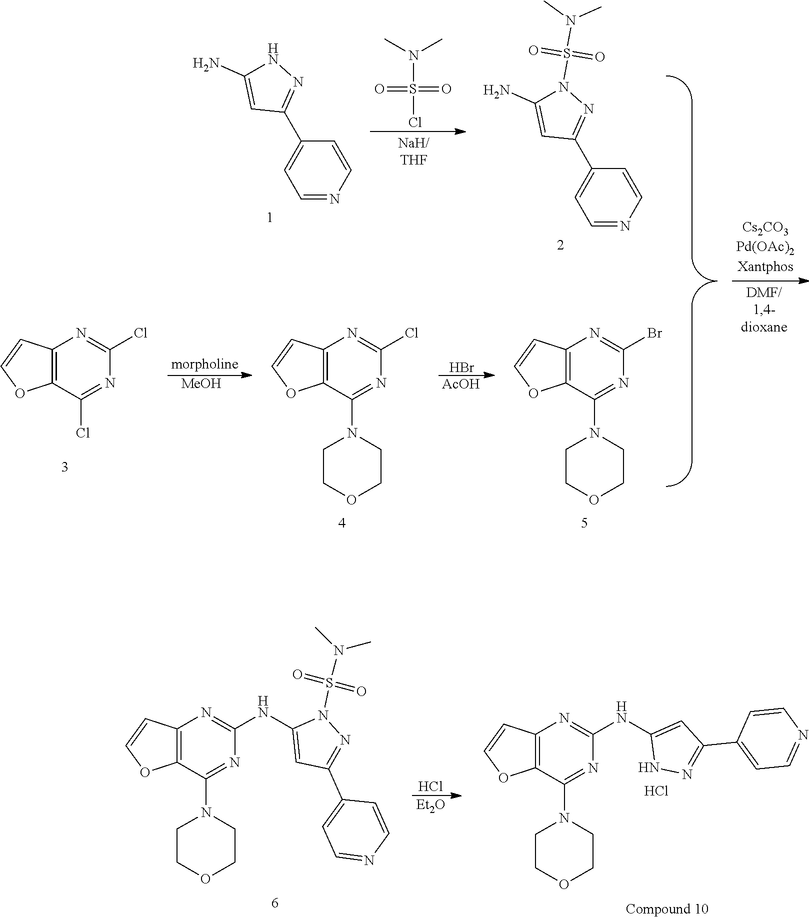

### 1.1) Synthesis of S-amino-N,N-dimethyl-3-(pyridin-4-yl)-1H-pyrazole-1-sulfonamide

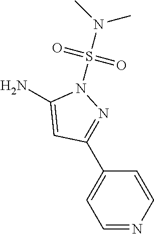 [SMILES: CN(C)S(=O)(=O)n1nc(-c2ccncc2)cc1N]

To a solution of 3-(pyridin-4-yl)-1H-pyrazol-5-amine (500 mg, 3.12 mmol) in THE (5 mL) at 0° C. was added NaH (374 mg, 9.36 mmol). After stirred at 0° C. for 1 h, to the solution was added dimethylsulfamoyl chloride (536 mg, 3.75 mmol). The completion of the reaction was monitored by TLC. The reaction mixture was quenched with a saturated NH₄Cl solution. The aqueous solution was extracted with ethyl acetate (3×50 mL). The combined organic phase was dried over anhydrous Na₂SO₄, filtrated and concentrated under reduced pressure. The resulting residue was purified by silica gel column chromatography with a gradient elution of 20% EtOAc/PE to 33% EtOAc/PE to provide 5-amino-N,N-dimethyl-3-(pyridin-4-yl)-1H-pyrazole-1-sulfonamide (94 mg, 0.35 mmol). LC-MS: m/z 268 (MH⁺). ¹HNMR (300 MHz, DMSO-d₆) δ 8.50 (d, J=6.0 Hz, 2H), 7.60 (dd, J=4.5, 1.2 Hz, 2H), 6.04 (s, 2H), 5.79 (s, 1H), 2.81 (s, 6H).

### 1.2) Synthesis of 2-chloro-4-morpholinofuro[3,2-d]pyrimidine

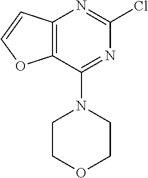 [SMILES: Clc1nc(N2CCOCC2)c2occc2n1]

To a solution of 2,4-dichlorofuro[3,2-d]pyrimidine (6.46 g, 34.2 mmol mmol) in methanol (100 mL) was added morpholine (5.95 g, 68.4 mmol). The reaction was stirred at room temperature for 30 min. The completion of the reaction was monitored by TLC. The reaction mixture was concentrated directly and the resulting residue was purified by silica gel column chromatography with a gradient elution of 10% EtOAc/PE to 20% EtOAc/PE to provide 2-chloro-4-morpholinofuro [3,2-d]pyrimidine (7.5 g, 40.1 mmol) as a white solid. LC-MS (ESI+): m/z 240/242 (MH⁺). ¹HNMR (300 MHz, CDCl₃) δ 7.74 (d, J=1.8 Hz, 1H), 6.79 (d, J=2.1 Hz, 1H), 4.05-4.02 (m, 4H), 3.85-3.82 (m, 4H).

### 1.3) Synthesis of 2-bromo-4-morpholinofuro[3,2-d]pyrimidine

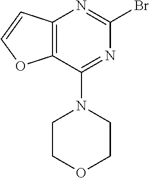 [SMILES: Brc1nc(N2CCOCC2)c2occc2n1]

A solution of 2-chloro-4-morpholinofuro[3,2-d]pyrimidine (180 mg, 0.75 mmol) in HBr/AcOH (33 wt. % in Acetic acid, 3 mL) was heated to reflux for 3.5 h. The completion of the reaction was monitored by LC-MS. The reaction mixture was quenched with a saturated NaHCO₃ solution and the pH was adjusted to 8. The aqueous solution was extracted with DCM/MeOH (15/1, 3×20 mL). The combined organic phase was dried over anhydrous Na₂SO₄, filtrated and concentrated under reduced pressure to provide 175 mg of crude 2-bromo-4-morpholino-furo[3,2-d]pyrimidine as a yellow solid. The crude product was used directly for the next step without further purification. LC-MS (ESI+): m/z 284/286 (MH⁺). ¹HNMR (300 MHz, CDCl₃) δ 7.72 (d, J=2.1 Hz, 1H), 6.79 (d, J=2.1 Hz, 1H), 4.04-4.01 (m, 4H), 3.85-3.81 (m, 4H).

### 1.4) Synthesis of N,N-dimethyl-5-((4-morpholinofuro[3,2-d]pyrimidin-2-yl)amino)-3-(pyridin-4-yl)-1H-pyrazole-1-sulfonamide

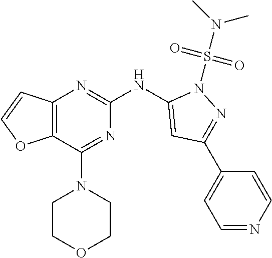 [SMILES: CN(C)S(=O)(=O)n1nc(-c2ccncc2)cc1Nc1nc(N2CCOCC2)c2occc2n1]

A suspension of 2-bromo-4-morpholinofuro[3,2-d]pyrimidine (48 mg, 0.17 mmol), 5-amino-N,N-dimethyl-3-(pyridin-4-yl)-1H-pyrazole-1-sulfonamide (54 mg, 0.20 mmol), Cs₂CO₃ (126 mg, 0.39 mmol), Pd(OAc)₂ (4 mg, 0.017 mmol) and Xantphos (10 mg, 0.017 mmol) in DMF/1,4-dioxane (1/7, 3 mL) was heated to 100° C. for 40 min under microwave conditions. The reaction mixture was concentrated directly and purified by silica gel column chromatography with a gradient elution of 1% MeOH/DCM to 5% MeOH/DCM to provide N,N-dimethyl-5-((4-morpholinofuro[3,2-d]pyrimidin-2-yl)amino)-3-(pyridin-4-yl)-1H-pyrazole-1-sulfonamide (27 mg, 0.06 mmol) as a yellow solid. LC-MS (ESI+): m/z 471 (MH⁺). ¹HNMR (300 MHz, CDCl₃) δ 8.69-8.67 (m, 3H), 7.77 (d, J=6.0 Hz, 2H), 7.70 (d, J=2.1 Hz, 1H), 7.35 (s, 1H), 6.79 (d, J=2.1 Hz, 1H), 4.05-4.02 (m, 4H), 3.87-3.84 (m, 4H), 3.08 (s, 6H).

### 1.5) Synthesis of 4-morpholino-N-(3-(pyridin-4-yl)-1H-pyrazol-5-yl)furo[3,2-d]pyrimidin-2-amine hydrochloride

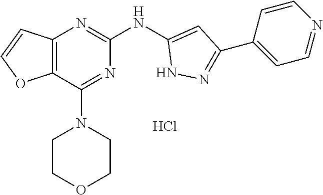 [SMILES: c1cc(-c2cc(Nc3nc(N4CCOCC4)c4occc4n3)[nH]n2)ccn1 ; Cl]

To a solution of N,N-dimethyl-5-((4-morpholinofuro[3,2-d]pyrimidin-2-yl)amino)-3-(pyridin-4-yl)-1H-pyrazole-1-sulfonamide (27 mg, 0.06 mmol) in DCM (4 mL) was added HCl/Et₂O (2 mL). The reaction mixture was stirred at room temperature for 2 h. After concentration and slurry in MeOH/Et₂O (1/20, 2 mL), 4-morpholino-N-(3-(pyridin-4-yl)-1H-pyrazol-5-yl)furo[3,2-d]pyrimidin-2-amine hydrochloride (Compound 10, 21.2 mg, 0.042 mmol) was obtained as a yellow solid. LC-MS (ESI+): m/z 364 (MH⁺). ¹HNMR (300 MHz, CD₃OD) δ 8.90 (d, J=6.9 Hz, 2H), 8.42 (d, J=6.9 Hz, 2H), 8.17 (d, J=2.1 Hz, 1H), 7.06-7.04 (m, 2H), 4.22-4.10 (m, 4H), 3.92-3.86 (m, 4H).

### Example 3: Compound 11 Using General Synthetic Route 3

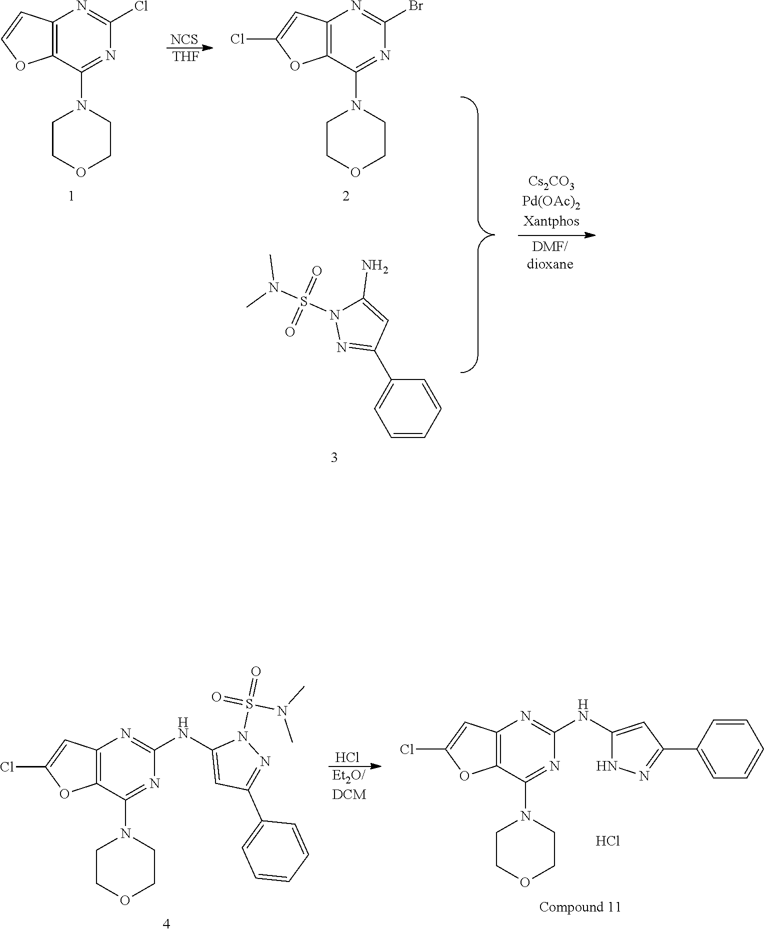

### 1.1) Synthesis of 2-bromo-6-chloro-4-morpholinofuro[3,2-d]pyrimidine

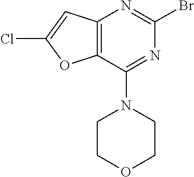 [SMILES: Clc1cc2nc(Br)nc(N3CCOCC3)c2o1]

To a solution of 2-bromo-4-morpholinofuro[3,2-d]pyrimidine (1.3 g, 4.59 mmol) in dry THF (4 mL) at −78° C. was added LDA (7.5 mL, 14.7 mmol) dropwise. After addition, the solution was stirred at that temperature for 1 h. Then to the solution was added NCS (733 mg, 5.5 mmol). The completion of the reaction was monitored by TLC. The reaction mixture was quenched with water (30 mL). The aqueous solution was extracted with EtOAc (3×30 mL). The combined organic phase was dried over anhydrous Na₂SO₄, filtrated and concentrated under reduced pressure. The residue was purified by silica gel column chromatography with a gradient elution of 10% EtOAc/PE to 25% EtOAc/PE to provide 2-bromo-6-chloro-4-morpholinofuro[3,2-d]pyrimidine (550 mg, 1.73 mmol) as a light yellow solid. LC-MS (ESI+): m/z 318/320 (MH⁺). ¹HNMR (300 MHz, CD₃OD) δ6.77 (s, 1H), 3.99-3.95 (m, 4H), 3.82-3.79 (m, 4H).

### 1.2) Synthesis of 3-((6-chloro-4-morpholinofuro[3,2-d]pyrimidin-2-yl)amino)-N,N-dimethyl-5-phenyl-1H-pyrazole-1-sulfonamide

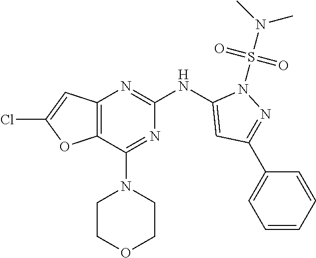 [SMILES: CN(C)S(=O)(=O)n1nc(-c2ccccc2)cc1Nc1nc(N2CCOCC2)c2oc(Cl)cc2n1]

A suspension of 2-bromo-6-chloro-4-morpholinofuro[3,2-d]pyrimidine (3×50 mg, 0.16 mmol), 3-amino-N,N-dimethyl-5-phenyl-1H-pyrazole-1-sulfonamide (42 mg, 0.16 mmol), Cs₂CO₃ (118 mg, 0.36 mmol), Pd(OAc)₂ (3.5 mg, 0.016 mmol) and Xantphos (9 mg, 0.015 mmol) in DMF/1,4-dioxane (1/7, 3 mL) was heated to 80° C. for 40 min under microwave conditions. The reaction mixture was concentrated directly and purified by silica gel column chromatography with a gradient elution of 10% EtOAc/PE to 35% EtOAc/PE to provide the impure product. After further preparative HPLC purification, 26 mg of pure 3-((6-chloro-4-morpholinofuro [3,2-d]pyrimidin-2-yl)amino)-N,N-dimethyl-5-phenyl-1H-pyrazole-1-sulfonamide was obtained. LC-MS (ESI+): m/z 504/506 (MH⁺). ¹HNMR (300 MHz, CDCl₃) δ 8.67 (s, 1H), 7.89 (dd, J=8.1, 1.5 Hz, 2H), 7.46-7.39 (m, 3H), 7.21 (s, 1H), 6.60 (s, 1H), 3.99-3.96 (m, 4H), 3.87-3.84 (m, 4H), 3.06 (s, 6H).

### 1.3) Synthesis of 6-chloro-4-morpholino-N-(5-phenyl-1H-pyrazol-3-yl)furo[3,2-d]pyrimidin-2-amine hydrochloride

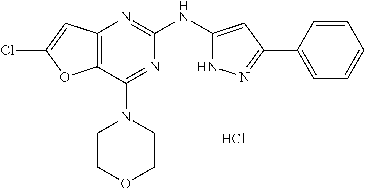 [SMILES: Clc1cc2nc(Nc3cc(-c4ccccc4)n[nH]3)nc(N3CCOCC3)c2o1 ; Cl]

To a solution of 3-((6-chloro-4-morpholinofuro[3,2-d]pyrimidin-2-yl)amino)-N,N-dimethyl-5-phenyl-1H-pyrazole-1-sulfonamide (26 mg, 0.05 mmol) in DCM (4 mL) was added HCl/Et₂O (2 mL). The reaction mixture was stirred at room temperature for 2 h. After concentration and slurry in MeOH/Et₂O (1/20, 2 mL), 6-chloro-4-morpholino-N-(5-phenyl-1H-pyrazol-3-yl)furo[3,2-d]pyrimidin-2-amine hydrochloride (Compound 11, 18.5 mg, 0.043 mmol) was obtained. LC-MS (ESI+): m/z 397/399 (MH⁺). ¹HNMR (300 MHz, CD₃OD) δ 7.87-7.69 (m, 2H), 7.51-7.39 (m, 3H), 7.09 (s, 1H), 6.41 (s, 1H), 4.17-4.12 (m, 4H), 3.89-3.84 (m, 4H).

### Example 4: Compound 12 Using General Synthetic Route 4

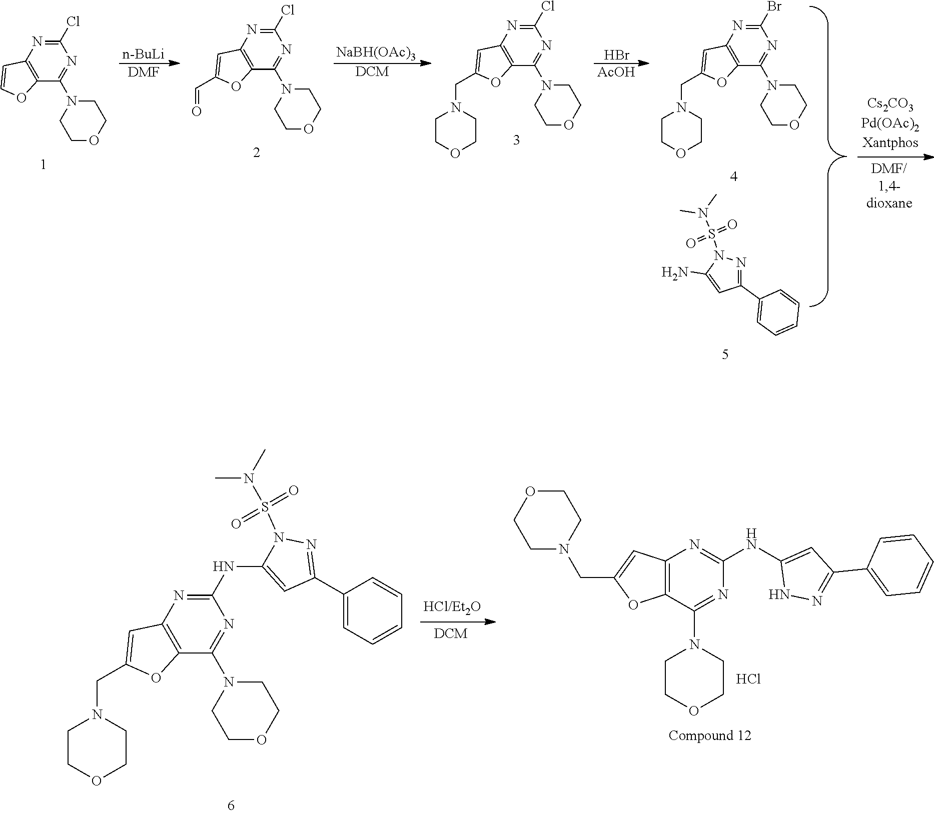

### 1.1) Synthesis of 2-chloro-4-morpholinofuro[3,2-d]pyrimidine-6-carbaldehyde

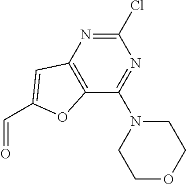 [SMILES: O=Cc1cc2nc(Cl)nc(N3CCOCC3)c2o1]

To a solution of 2-chloro-4-morpholinofuro[3,2-d]pyrimidine (50 mg, 0.21 mmol) in THE at −78° C. under N₂ was added n-BuLi (0.1 mL, 0.25 mmol). The mixture was stirred at that temperature for 15 min and then DMF (90 mg, 1.23 mmol) was added. The solution was allowed to warm to room temperature for 10 min. The completion of the reaction was monitored by TLC. The reaction was quenched with water and the aqueous solution was extracted with EtOAc (3×10 mL). The combined organic phase was dried over anhydrous Na₂SO₄, filtrated and concentrated under reduced pressure. The resulting residue was purified by silica gel column chromatography with a gradient elution of 10% EtOAc/PE to 35% EtOAc/PE to provide 2-chloro-4-morpholinofuro[3,2-d]pyrimidine-6-carbaldehyde (26 mg, 0.097 mmol) as a white solid. LC-MS (ESI+): m/z 268/270 (MH⁺). ¹HNMR (300 MHz, CDCl₃) δ 9.91 (s, 1H), 7.48 (s, 1H), 4.15-4.10 (m, 4H), 3.88-3.85 (m, 4H).

### 1.2) Synthesis of 2-chloro-4-morpholino-6-(morpholinomethyl)furo[3,2-d]pyrimidine

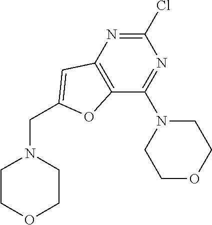 [SMILES: Clc1nc(N2CCOCC2)c2oc(CN3CCOCC3)cc2n1]

A solution of 2-chloro-4-morpholinofuro[3,2-d]pyrimidine-6-carbaldehyde (150 mg, 0.56 mmol) and morpholine (58 mg, 0.67 mmol) in DCM was stirred at room temperature for 15 min. To the solution was added sodium triacetoxyborohydride (356 mg, 1.68 mmol). The mixture was stirred at room temperature for 3 h. The completion of the reaction was monitored by TLC. The reaction mixture was quenched with a saturated NaHCO₃ solution and the pH was adjusted to 8. The aqueous solution was extracted with DCM/MeOH (15/1, 3×20 mL). The combined organic phase was dried over anhydrous Na₂SO₄, filtrated and concentrated under reduced pressure. The resulting residue was purified by silica gel column chromatography with a gradient elution of 30% EtOAc/PE to EtOAc to provide 2-chloro-4-morpholino-6-(morpholinomethyl)furo[3,2-d]pyrimidine (120 mg, 0.35 mmol). LC-MS (ESI+): m/z 339/341 (MH⁺). ¹HNMR (300 MHz, CDCl₃) δ 6.63 (s, 1H), 4.13-3.92 (m, 4H), 3.85-3.82 (m, 4H), 3.74-3.71 (m, 4H), 3.63 (s, 2H), 2.56-2.53 (m, 4H).

### 1.3) Synthesis of 2-bromo-4-morpholino-6-(morpholinomethyl)furo[3,2-d]pyrimidine

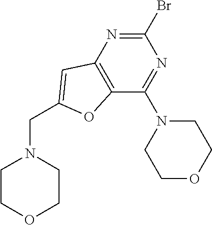 [SMILES: Brc1nc(N2CCOCC2)c2oc(CN3CCOCC3)cc2n1]

A solution of 2-chloro-4-morpholino-6-(morpholinomethyl)furo[3,2-d]pyrimidine (120 mg, 0.35 mmol) in HBr/AcOH (33 wt. % in Acetic acid, 5 mL) was heated to refluxed for 3.5 h. The completion of the reaction was monitored by LC-MS. The reaction mixture was quenched with a saturated NaHCO₃ solution and the pH was adjusted to 8. The aqueous solution was extracted with DCM/MeOH (15/1, 3×20 mL). The combined organic phase was dried over anhydrous Na₂SO₄, filtrated and concentrated under reduced pressure to provide 80 mg of crude 2-bromo-4-morpholino-6-(morpholinomethyl)furo[3,2-d]pyrimidine as a brown solid. The crude product was used directly for the next step without further purification. LC-MS (ESI+): m/z 383/385 (MH⁺). ¹HNMR (300 MHz, CD₃OD) δ 6.71 (s, 1H), 4.03-4.00 (m, 4H), 3.83-3.81 (m, 4H), 3.75 (s, 2H), 3.72-3.68 (m, 4H), 2.57-2.54 (m, 4H).

### 1.4) Synthesis of N,N-dimethyl-5-((4-morpholino-6-(morpholinomethyl)furo[3,2-d]pyrimidin-2-yl)amino)-3-phenyl-1H-pyrazole-1-sulfonamide

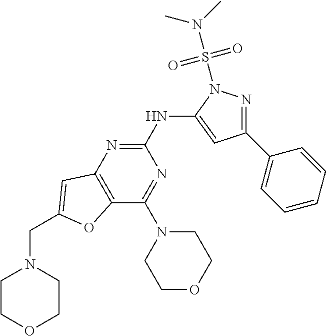 [SMILES: CN(C)S(=O)(=O)n1nc(-c2ccccc2)cc1Nc1nc(N2CCOCC2)c2oc(CN3CCOCC3)cc2n1]

A suspension of 2-bromo-4-morpholino-6-(morpholinomethyl)furo[3,2-d]pyrimidine (60 mg, 0.16 mmol), 5-amino-N,N-dimethyl-3-phenyl-1H-pyrazole-1-sulfonamide (50 mg, 0.19 mmol), Cs₂CO₃ (120 mg, 0.37 mmol), Pd(OAc)₂ (3 mg, 0.01 mmol) and Xantphos (6 mg, 0.01 mmol) in DMF/1,4-dioxane (1/7, 8 mL) was heated to 90° C. for 30 min under microwave condition. The reaction mixture was concentrated directly and purified by silica gel column chromatography with a gradient elution of 1% MeOH/DCM to 5% MeOH/DCM to provide N,N-dimethyl-5-((4-morpholino-6-(morpholinomethyl)furo[3,2-d]pyrimidin-2-yl)amino)-3-phenyl-1H-pyrazole-1-sul-fonamide (23 mg, 0.04 mmol) as a yellow solid. LC-MS (ESI+): m/z 569 (MH⁺). ¹HNMR (300 MHz, CDCl₃) δ 8.65 (s, 1H), 7.89 (d, J=6.9 Hz, 2H), 7.45-7.38 (m, 3H), 7.25 (s, 1H), 6.63 (s, 1H), 4.02-4.00 (m, 4H), 3.87-3.84 (m, 4H), 3.75-3.73 (m, 4H), 3.67 (s, 2H), 3.06 (s, 6H), 2.58-2.55 (m, 4H).

### 1.5) Synthesis of 4-morpholino-6-(morpholinomethyl)-N-(3-phenyl-1H-pyrazol-5-yl)furo[3,2-d]pyrimidin-2-amine hydrochloride

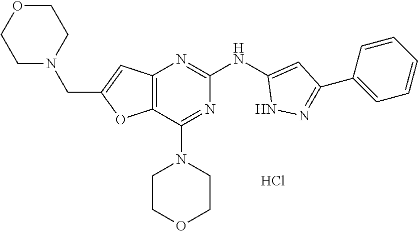 [SMILES: c1ccc(-c2cc(Nc3nc(N4CCOCC4)c4oc(CN5CCOCC5)cc4n3)[nH]n2)cc1 ; Cl]

To a solution of N,N-dimethyl-5-((4-morpholino-6-(morpholinomethyl)furo[3,2-d]pyrimidin-2-yl)amino)-3-phenyl-1H-pyrazole-1-sulfonamide (23 mg, 0.04 mmol) in DCM (4 mL) was added HCl/Et₂O (2 mL). The reaction mixture was stirred at room temperature for 2 h. After concentration and slurry in MeOH/Et₂O (1/20, 2 mL), 4-morpholino-6-(morpholinomethyl)-N-(3-phenyl-1H-pyrazol-5-yl)furo[3,2-d]pyrimidin-2-amine hydrochloride (Compound 12, 21.2 mg, 0.042 mmol) was obtained as a yellow solid. LC-MS (ESI+): m/z 462 (MH⁺). ¹HNMR (300 MHz, CD₃OD) δ 7.79-7.70 (m, 2H), 7.52-7.42 (m, 3H), 7.39 (s, 1H), 6.42 (s, 1H), 4.67 (s, 2H), 4.32-4.12 (m, 4H), 4.10-3.86 (m, 8H), 3.55-3.42 (m, 4H).

### Example 5: Compound 22 Using General Synthetic Route 5

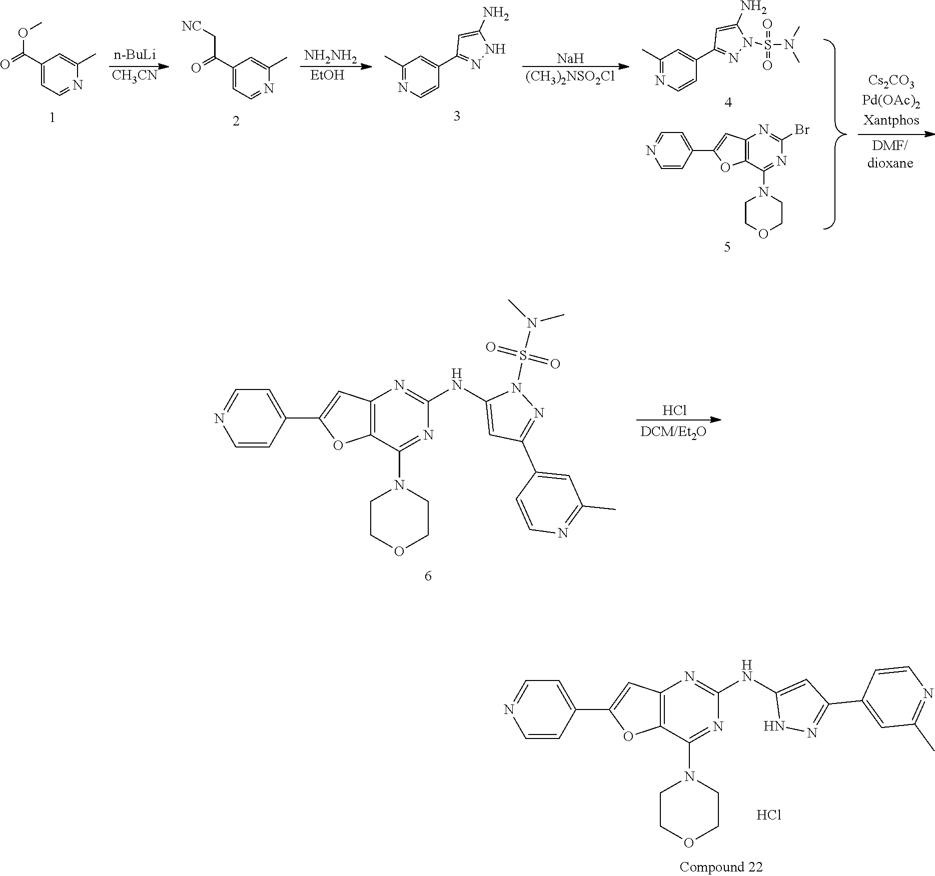

### 1.1) Synthesis of 3-(2-methylpyridin-4-yl)-3-oxopropanenitrile

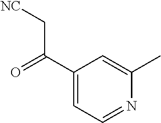

To a solution of acetonitrile (1.63 g, 39.7 mmol) in anhydrous THF (40 mL) at −70° C. under N₂ was added n-BuLi (15.9 mL, 39.7 mmol) dropwise. After addition, a solution of methyl 2-methylisonicotinate (2.0 g, 13.2 mmol) in THF (10 mL) was added to the above solution over 10 min. The reaction mixture was stirred at that temperature for 2 h. The completion of the reaction was monitored by TLC. The reaction was quenched with AcOH (6.9 mL) and the solution was concentrated directly under reduced pressure. The residue was purified by silica gel column chromatography with a gradient elution of 1% MeOH/DCM to 5% MeOH/DCM to provide 3-(2-methylpyridin-4-yl)-3-oxopropanenitrile (1.15 g, 7.2 mmol) as a yellow solid. LC-MS (ESI+): m/z 161 (MH⁺). ¹HNMR (300 MHz, CDCl₃) (8.77 (d, J=5.4 Hz, 1H), 7.58 (s, 1H), 7.51 (d, J=5.1 Hz, 1H), 4.08 (s, 2H), 2.69 (s, 3H).

### 1.2) Synthesis of 3-(2-methylpyridin-4-yl)-1H-pyrazol-5-amine

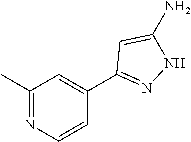

To a solution of 3-(2-methylpyridin-4-yl)-3-oxopropanenitrile (1.15 g, 7.2 mmol) in EtOH (40 mL) was added NH₂NH₂.H₂O (0.54 g, 10.8 mmol). The mixture was heated to reflux overnight. The completion of the reaction was monitored by TLC. The reaction mixture was concentrated directly and purified by silica gel column chromatography with a gradient elution of 1% MeOH/DCM to 5% MeOH/DCM to provide 3-(2-methylpyridin-4-yl)-1H-pyrazol-5-amine (0.8 g, 4.6 mmol) as a yellow oil. LC-MS (ESI+): m/z 175 (MH⁺). ¹HNMR (300 MHz, CDCl₃) δ 8.47 (d, J=5.4 Hz, 1H), 7.33 (s, 1H), 7.27 (d, J=5.1 Hz, 1H), 6.00 (s, 1H), 4.70 (brs, 2H), 2.55 (s, 3H).

### 1.3) Synthesis of 5-amino-N,N-dimethyl-3-(2-methylpyridin-4-yl)-1H-pyrazole-1-sulfonamide

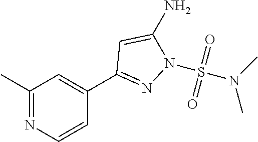

To a solution of 3-(2-methylpyridin-4-yl)-1H-pyrazol-5-amine (800 mg, 4.6 mmol) in THF (30 mL) at 0° C. was added NaH (413 mg, 6.9 mmol). After stirred at 0° C. for 1 h, to the reaction solution was added dimethylsulfamoyl chloride (854 mg, 5.98 mmol). The completion of the reaction was monitored by TLC. The reaction mixture was quenched with a saturated NH₄Cl solution. The aqueous solution was extracted with DCM/MeOH (15/1, 3×50 mL). The combined organic phase was dried over anhydrous Na₂SO₄, filtrated and concentrated under reduced pressure. The resulting residue was purified by silica gel column chromatography with a gradient elution of 50% EtOAc/PE to 66% EtOAc/PE to provide 5-amino-N,N-dimethyl-3-(2-methylpyridin-4-yl)-1H-pyrazole-1-sulfonamide (220 mg, 0.78 mmol). LC-MS: m/z 282 (MH⁺). ¹HNMR (300 MHz, CDCl₃) δ 8.51 (d, J=5.1 Hz, 1H), 7.52 (s, 1H), 7.43 (d, J=5.4 Hz, 1H), 5.78 (s, 1H), 4.91 (brs, 2H), 3.05 (s, 6H), 2.60 (s, 3H).

### 1.4) Synthesis of N,N-dimethyl-3-(2-methylpyridin-4-yl)-5-((4-morpholino-6-(pyridin-4-yl)furo[3,2-d]pyrimidin-2-yl)amino)-1H-pyrazole-1-sulfonamide

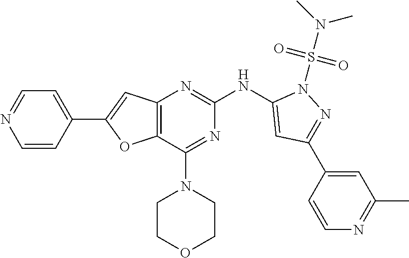 [SMILES: Cc1cc(-c2cc(Nc3nc(N4CCOCC4)c4oc(-c5ccncc5)cc4n3)n(S(=O)(=O)N(C)C)n2)ccn1]

A suspension of 2-bromo-4-morpholino-6-(pyridin-4-yl)furo[3,2-d]pyrimidine (2×50 mg, 0.14 mmol), 5-amino-N,N-dimethyl-3-(2-methylpyridin-4-yl)-1H-pyrazole-1-sulfonamide (46.8 mg, 0.17 mmol), Cs₂CO₃ (90.6 mg, 0.28 mmol), Pd(OAc)₂ (3 mg, 0.014 mmol) and Xantphos (6 mg, 0.014 mmol) in DMF/1,4-dioxane (1/7, 4 mL) was heated to 100° C. for 30 min under microwave condition. The reaction mixture was concentrated directly and purified by silica gel column chromatography with a gradient elution of 1% MeOH/DCM to 5% MeOH/DCM to provide impure N,N-dimethyl-3-(2-methylpyridin-4-yl)-5-((4-morpholino-6-(pyridin-4-yl)furo [3,2-d]pyrimidin-2-yl)amino)-1H-pyrazole-1-sulfonamide (23.2 mg, 0.04 mmol) as a yellow solid. LC-MS (ESI+): m/z 562 (MH⁺). ¹HNMR (300 MHz, CDCl₃) β 8.76 (d, J=6.0 Hz, 2H), 8.71 (s, 1H), 8.57 (d, J=5.4 Hz, 1H), 7.66-7.61 (m, 3H), 7.58 (d, J=4.2 Hz, 1H), 7.35 (s, 1H), 4.10-4.08 (m, 4H), 3.93-3.90 (m, 4H), 3.09 (s, 6H), 2.65 (s, 3H).

### 1.5) Synthesis of N-(3-(2-methylpyridin-4-yl)-1H-pyrazol-5-yl)-4-morpholino-6-(pyridin-4-yl)furo[3,2-d]pyrimidin-2-amine hydrochloride

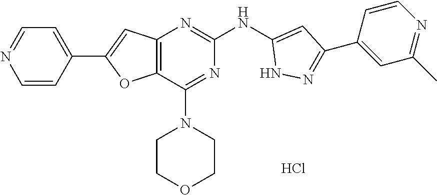 [SMILES: Cc1cc(-c2cc(Nc3nc(N4CCOCC4)c4oc(-c5ccncc5)cc4n3)[nH]n2)ccn1 ; Cl]

To a solution of impure N,N-dimethyl-3-(2-methylpyridin-4-yl)-5-((4-morpholino-6-(pyridin-4-yl) furo[3,2-d]pyrimidin-2-yl)amino)-1H-pyrazole-1-sulfonamide (23 mg, 0.04 mmol) in DCM (4 mL) was added HCl/Et₂O (2 mL). The reaction mixture was stirred at room temperature for 2 h. After concentration and slurry in MeOH/Et₂O (1/20, 2 mL), N-(3-(2-methylpyridin-4-yl)-1H-pyrazol-5-yl)-4-morpholino-6-(pyridin-4-yl)furo[3,2-d]pyrimidin-2-amine hydrochloride (Compound 22, 20.8 mg, 0.04 mmol) was obtained as a yellow solid. LC-MS (ESI+): m/z 455 (MH⁺). ¹HNMR (300 MHz, CD₃OD) δ 8.94 (d, J=6.6 Hz, 2H), 8.74 (d, J=6.6 Hz, 1H), 8.51 (d, J=6.9 Hz, 2H), 8.30 (s, 1H), 8.24 (d, J=6.3 Hz, 1H), 8.02 (s, 1H), 6.99 (s, 1H), 4.32-4.28 (m, 4H), 3.95-3.94 (m, 4H), 2.85 (s, 3H).

### Example 6: Compound 28 Using General Synthetic Route 6

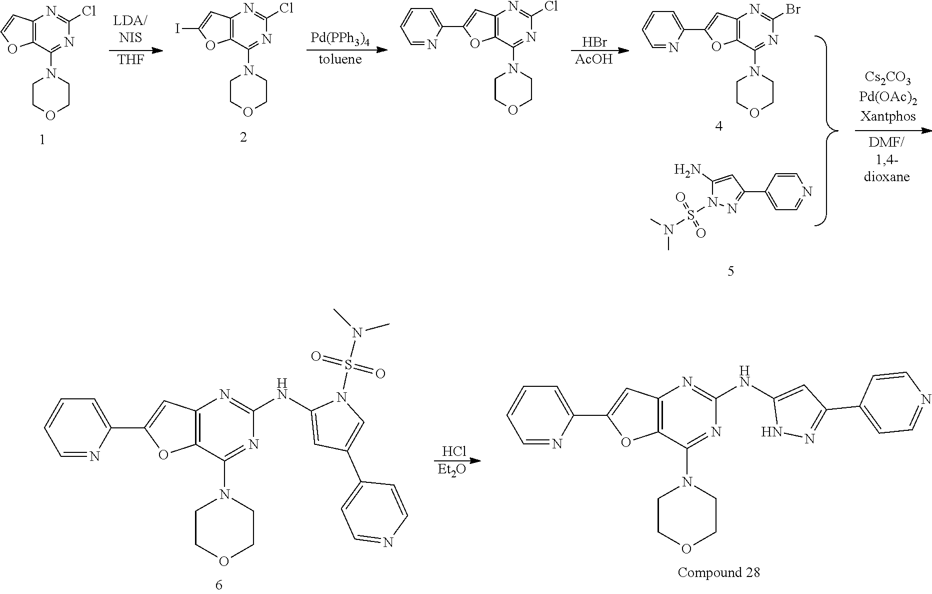

### 1.1) Synthesis of 2-chloro-6-iodo-4-morpholinofuro[3,2-d]pyrimidine

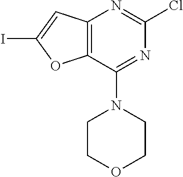 [SMILES: Clc1nc(N2CCOCC2)c2oc(I)cc2n1]

To a solution of 2-chloro-4-morpholinofuro[3,2-d]pyrimidine (2.0 g, 0.83 mmol) in THF (30 mL) at −78° C. under N₂ was added LDA (1.33 mL, 2M, 2.66 mmol). After stirred at −78° C. for 1 h, to the solution was added NIS (2.25 g, 1.0 mmol) in THF (10 mL). The completion of the reaction was monitored by TLC. The reaction mixture was quenched with water (50 mL). The aqueous solution was extracted with DCM (3×50 mL). The combined organic phase was dried over anhydrous Na₂SO₄, filtrated and concentrated under reduced pressure. The resulting residue was purified by silica gel column chromatography with a gradient elution of 5% EtOAc/PE to 10% EtOAc/PE to provide 2-chloro-6-iodo-4-morpholinofuro[3,2-d]pyrimidine (1.6 g, 4.4 mmol) as yellow solid. LC-MS (ESI+): m/z 366/368 (MH⁺). ¹HNMR (300 MHz, CDCl₃) δ 6.97 (s, 1H), 4.01-3.98 (m, 4H), 3.85-3.82 (m, 4H).

### 1.2) Synthesis of 2-chloro-4-morpholino-6-(pyridin-2-yl)furo[3,2-d]pyrimidine

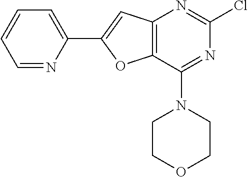 [SMILES: Clc1nc(N2CCOCC2)c2oc(-c3ccccn3)cc2n1]

A solution of 2-chloro-6-iodo-4-morpholinofuro[3,2-d]pyrimidine (1 g, 2.7 mmol), 2-(tributylstannyl)pyridine (1.2 g, 3.3 mmol) and Pd(PPh₃)₄ (155 mg, 0.14 mmol) in toluene (5 mL) was heated to 90° C. overnight. The completion was monitored by TLC. The reaction mixture was diluted with water and extracted with DCM/MeOH (15/1, 3×50 mL). The combined organic phase was dried over anhydrous Na₂SO₄, filtrated and concentrated under reduced pressure. The resulting residue was purified by silica gel column chromatography with a gradient elution of 1% MeOH/DCM to 5% MeOH/DCM to provide 2-chloro-4-morpholino-6-(pyridin-2-yl)furo[3,2-d]pyrimidine (352 mg, 1.11 mmol) as a yellow solid. LC-MS (ESI+): m/z 317/319 (MH⁺).

### 1.3) Synthesis of 2-bromo-4-morpholino-6-(pyridin-2-yl)furo[3,2-d]pyrimidine

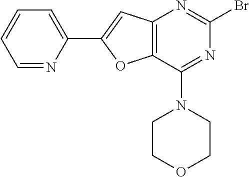 [SMILES: Brc1nc(N2CCOCC2)c2oc(-c3ccccn3)cc2n1]

A solution of 2-chloro-4-morpholino-6-(pyridin-2-yl)furo[3,2-d]pyrimidine (350 mg, 1.11 mmol) in HBr/AcOH (33 wt. % in Acetic acid, 5 mL) was heated to reflux for 3.5 h. The completion of the reaction was monitored by LC-MS. The reaction mixture was quenched with a saturated NaHCO₃ solution and the pH was adjusted to 8. The aqueous solution was extracted with DCM/MeOH (15/1, 3×20 mL). The combined organic phase was dried over anhydrous Na₂SO₄, filtrated and concentrated under reduced pressure to provide 290 mg of crude 2-bromo-4-morpholino-6-(pyridin-2-yl)furo[3,2-d]pyrimidine as a yellow solid. The crude product was used directly for the next step without further purification. LC-MS (ESI+): m/z 361/363 (MH⁺).

### 1.4) Synthesis of N,N-dimethyl-5-((4-morpholino-6-(pyridin-2-yl)furo[3,2-d]pyrimidin-2-yl)amino)-3-(pyridin-4-yl)-1H-pyrazole-1-sulfonamide

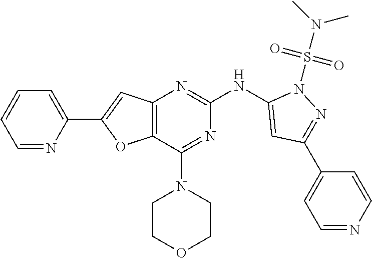 [SMILES: CN(C)S(=O)(=O)n1nc(-c2ccncc2)cc1Nc1nc(N2CCOCC2)c2oc(-c3ccccn3)cc2n1]

A suspension of 2-bromo-4-morpholino-6-(pyridin-2-yl)furo[3,2-d]pyrimidine (2×40 mg, 0.13 mmol), 3-amino-N,N-dimethyl-5-(pyridin-4-yl)-1H-pyrazole-1-sulfonamide (41 mg, 0.15 mmol), Cs₂CO₃ (95 mg, 0.29 mmol), Pd(OAc)₂ (3 mg, 0.013 mmol) and Xantphos (7 mg, 0.013 mmol) in DMF/1,4-dioxane (1/7, 4 mL) was heated to 90° C. for 30 min under microwave conditions. The reaction mixture was concentrated directly and purified by silica gel column chromatography with a gradient elution of 1% MeOH/DCM to 5% MeOH/DCM to provide N,N-dimethyl-5-((4-morpholino-6-(pyridin-2-yl)furo[3,2-d]pyrimidin-2-yl)amino)-3-(pyridin-4-yl)-1H-pyrazole-1-sulfonamide (35.6 mg, 0.065 mmol) as a yellow solid. LC-MS (ESI+): m/z 548 (MH⁺). ¹HNMR (300 MHz, CDCl₃) δ 8.70-8.68 (m, 3H), 7.81-7.77 (m, 4H), 7.41-7.39 (m, 2H), 7.36-7.26 (m, 1H), 4.11-4.09 (m, 4H), 3.92-3.91 (m, 4H), 3.09 (s, 6H).

### 1.5) Synthesis of Compound 28, 4-morpholino-6-(pyridin-2-yl)-N-(3-(pyridin-4-yl)-s1H-pyrazol-5-yl)furo[3,2-d]pyrimidin-2-aminehydrochloride

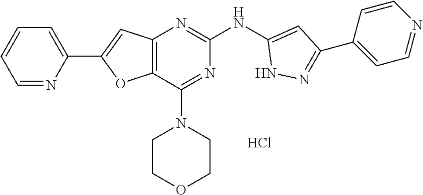 [SMILES: c1ccc(-c2cc3nc(Nc4cc(-c5ccncc5)n[nH]4)nc(N4CCOCC4)c3o2)nc1 ; Cl]

To a solution of N,N-dimethyl-5-((4-morpholino-6-(pyridin-2-yl)furo[3,2-d]pyrimidin-2-yl)amino)-3-(pyridin-4-yl)-1H-pyrazole-1-sulfonamide (35.6 mg, 0.065 mmol) in DCM (4 mL) was added HCl/Et₂O (2 mL). The reaction mixture was stirred at room temperature for 2 h. After concentration and slurry in MeOH/Et₂O (1/20, 2 mL), 4-morpholino-6-(pyridin-2-yl)-N-(3-(pyridin-4-yl)-1H-pyrazol-5-yl) furo[3,2-d] pyrimidin-2-aminehydrochloride (Compound 28, 28.8 mg, 0.06 mmol) was obtained as a yellow solid. LC-MS (ESI+): m/z 441 (MH⁺). ¹HNMR (300 MHz, CD₃OD) δ 8.92 (d, J=6.3 Hz, 2H), 8.75 (d, J=4.8 Hz, 1H), 8.45 (d, J=6.6 Hz, 2H), 8.17 (d, J=7.2 Hz, 1H), 8.12-8.07 (m, 1H), 7.65 (s, 1H), 7.62-7.58 (m, 1H), 7.07 (s, 1H), 4.47-4.13 (m, 4H), 3.97-3.85 (m, 4H).

### Example 7: Compound 34 Using General Synthetic Route 7

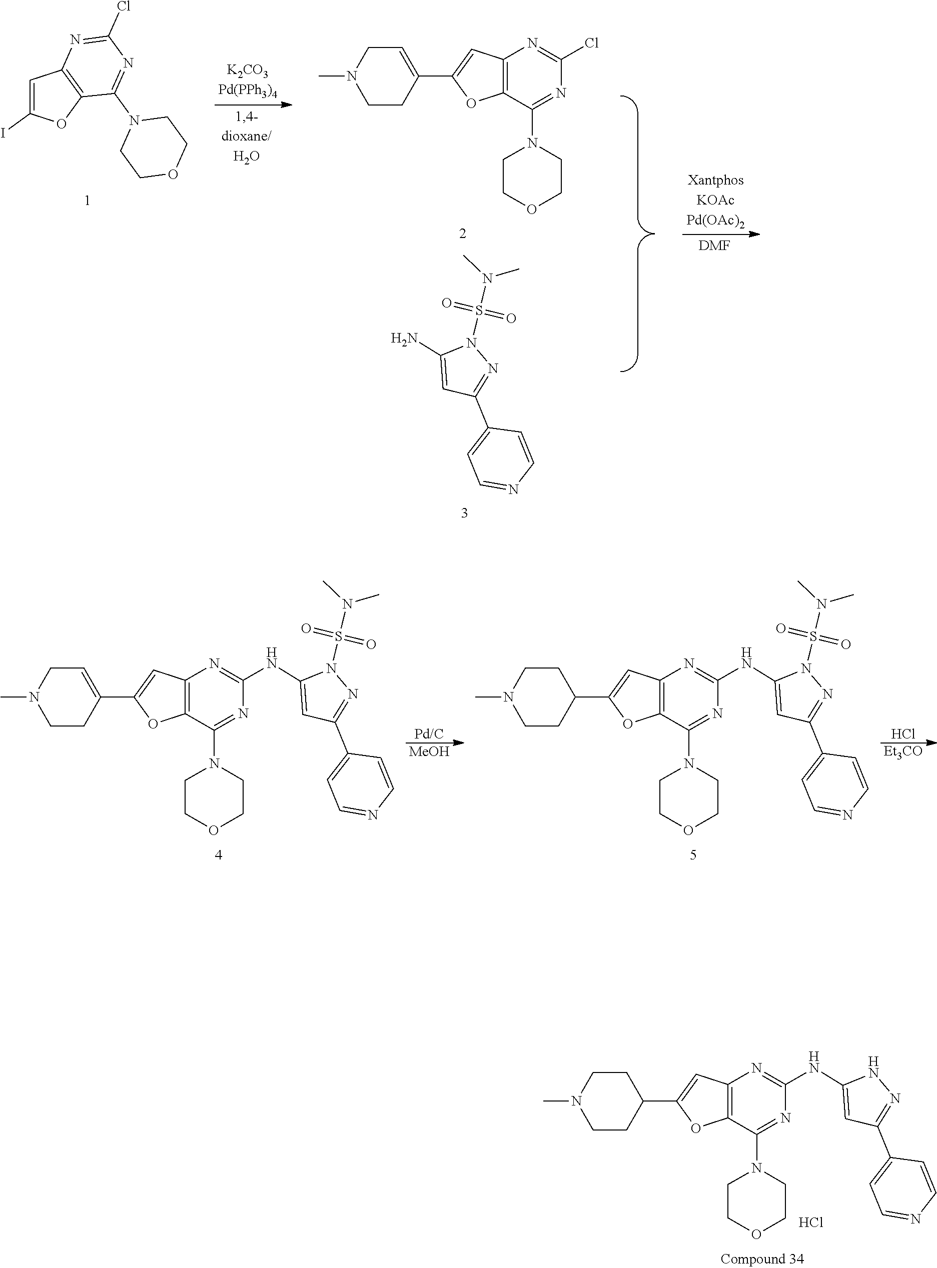

### 1.1) Synthesis of 2-chloro-6-(1-methyl-1,2,3,6-tetrahydropyridin-4-yl)-4-morpholinofuro[3,2-d]pyrimidine

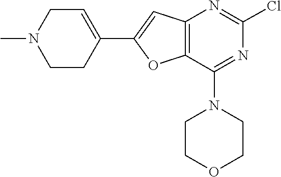 [SMILES: CN1CC=C(c2cc3nc(Cl)nc(N4CCOCC4)c3o2)CC1]

A suspension of 2-chloro-6-iodo-4-morpholinofuro[3,2-d]pyrimidine (400 mg, 1.1 mmol), 1-methyl-4-(4,4,5,5-tetramethyl-1,3,2-dioxaborolan-2-yl)-1,2,3,6-tetrahydropyridine (244 mg, 1.1 mmol), K₂CO₃ (454 mg, 3.29 mmol) and Pd(PPh₃)₄ (127 mg, 0.011 mmol) in 1,4-dioxane/H₂O (8/1, 40 mL) was heated to 50° C. for 2 h under N₂. The completion was monitored by TLC. The reaction was diluted with water and extracted with DCM/MeOH (15/1, 3×30 mL). The combined organic phase was dried over Na₂SO₄, filtrated and concentrated under reduced pressure. The residue was purified by silica gel column chromatography with a gradient elution of 1% MeOH/DCM to 3% MeOH/DCM to provide 2-chloro-6-(1-methyl-1,2,3,6-tetrahydropyridin-4-yl)-4-morpholinofuro[3,2-d]pyrimidine (270 mg, 0.81 mmol) as a yellow solid. LC-MS (ESI+): m/z 335/337 (MH⁺). ¹HNMR (300 MHz, CDCl₃) δ 6.52 (s, 1H), 6.52-6.47 (m, 1H), 4.03-4.00 (m, 4H), 3.86-3.83 (m, 4H), 3.19-3.18 (m, 2H), 2.70-2.66 (m, 2H), 2.58-2.54 (m, 2H), 2.43 (s, 3H).

### 1.2) Synthesis of N,N-dimethyl-5-((6-(1-methyl-1,2,3,6-tetrahydropyridin-4-yl)-4-morpholinofuro[3,2-d]pyrimidin-2-yl)amino)-3-(pyridin-4-yl)-1H-pyrazole-1-sulfonamide

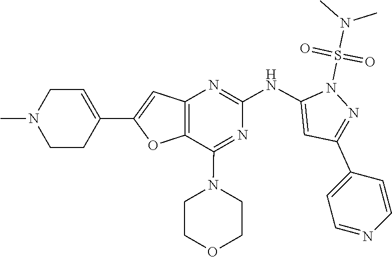 [SMILES: CN1CC=C(c2cc3nc(Nc4cc(-c5ccncc5)nn4S(=O)(=O)N(C)C)nc(N4CCOCC4)c3o2)CC1]

A suspension of 2-chloro-6-(1-methyl-1,2,3,6-tetrahydropyridin-4-yl)-4-morpholinofuro [3,2-d]pyrimidine (120 mg, 0.60 mmol), 5-amino-N,N-dimethyl-3-(pyridin-4-yl)-1H-pyrazole-1-sulfonamide (176 mg, 0.18 mmol), KOAc (176 mg, 1.80 mmol), Pd(OAc)₂ (14.8 mg, 0.066 mmol) and Xantphos (80 mg, 0.18 mmol) in DMF (20 mL) was heated to 80° C. for 3 h. The reaction mixture was diluted with water and extracted with DCM/MeOH (15/1, 3×30 mL). The combined organic phase was dried over anhydrous Na₂SO₄, filtrated and concentrated under reduced pressure. The residue was purified by silica gel column chromatography with a gradient elution of 1% MeOH/DCM to 10% MeOH/DCM to provide N,N-dimethyl-5-((6-(1-methyl-1,2,3,6-tetrahydropyridin-4-yl)-4-morpholinofuro[3,2-d]pyrimidin-2-yl)amino)-3-(pyridin-4-yl)-1H-pyrazole-1-sulfonamide (120 mg, 0.21 mmol) as a yellow solid. LC-MS (ESI+): m/z 566 (MH⁺). ¹HNMR (300 MHz, CDCl₃) δ 8.68 (d, J=6.0 Hz, 2H), 7.77 (d, J=6.0 Hz, 2H), 7.32 (s, 1H), 6.58 (s, 1H), 6.47-6.42 (m, 1H), 4.03-4.00 (m, 4H), 3.88-3.85 (m, 4H), 3.30-3.29 (m, 2H), 3.07 (s, 6H), 2.83-2.79 (m, 2H), 2.62-2.58 (m, 2H), 2.50 (s, 3H).

### 1.3) Synthesis of N,N-dimethyl-5-((6-(1-methylpiperidin-4-yl)-4-morpholinofuro[3,2-d]pyrimidin-2-yl)amino)-3-(pyridin-4-yl)-1H-pyrazole-1-sulfonamide

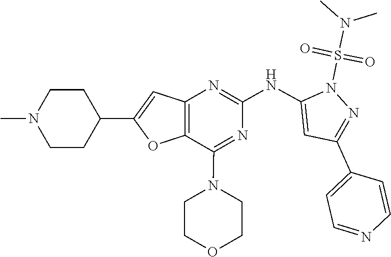 [SMILES: CN1CCC(c2cc3nc(Nc4cc(-c5ccncc5)nn4S(=O)(=O)N(C)C)nc(N4CCOCC4)c3o2)CC1]

To a solution of N,N-dimethyl-5-((6-(1-methyl-1,2,3,6-tetrahydropyridin-4-yl)-4-morpholinofuro [3,2-d]pyrimidin-2-yl)amino)-3-(pyridin-4-yl)-1H-pyrazole-1-sulfonamide (120 mg, 0.21 mmol) in DCM/MeOH (1/1, 10 mL) was added Pd/C under H₂ (balloon). The reaction mixture was stirred at room temperature for 3 h. The completion of the reaction was monitored by LC-MS. After filtration, the filtrate was concentrated directly and purified by silica gel column chromatography with a gradient elution of 1% MeOH/DCM to 10% MeOH/DCM to provide N,N-dimethyl-5-((6-(1-methylpiperidin-4-yl)-4-morpholinofuro[3,2-d]pyrimidin-2-yl)amino)-3-(pyridin-4-yl)-1H-pyrazole-1-sulfonamide (71 mg, 0.13 mmol) as a white solid. LC-MS (ESI+): m/z 568 (MH⁺). ¹HNMR (300 MHz, CD₃OD) δ 8.62 (d, J=6.3 Hz, 2H), 7.87 (d, J=6.0 Hz, 2H), 7.29 (s, 1H), 6.54 (s, 1H), 4.03-4.00 (m, 4H), 3.86-3.83 (m, 4H), 3.40-3.34 (m, 2H), 3.06 (s, 6H), 2.82-2.79 (m, 2H), 2.67 (s, 3H), 2.30-2.26 (m, 2H), 1.97-1.95 (m, 2H).

### 1.4) Synthesis of 6-(1-methylpiperidin-4-yl)-4-morpholino-N-(3-(pyridin-4-yl)-1H-pyrazol-5-yl)furo[3,2-d]pyrimidin-2-amine hydrochloride

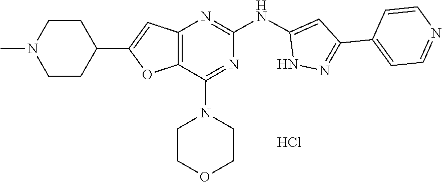 [SMILES: CN1CCC(c2cc3nc(Nc4cc(-c5ccncc5)n[nH]4)nc(N4CCOCC4)c3o2)CC1 ; Cl]

To a solution of N,N-dimethyl-5-((6-(1-methylpiperidin-4-yl)-4-morpholinofuro[3,2-d]pyrimidin-2-yl)amino)-3-(pyridin-4-yl)-1H-pyrazole-1-sulfonamide (71 mg, 0.13 mmol) in DCM (4 mL) was added HCl/Et₂O (2 mL). The reaction mixture was stirred at room temperature for 2 h. After concentration and slurry in MeOH/Et₂O (1/20, 2 mL), 6-(1-methylpiperidin-4-yl)-4-morpholino-N-(3-(pyridin-4-yl)-1H-pyrazol-5-yl)furo[3,2-d]pyrimidin-2-amine hydrochloride (Compound 34, 55 mg, 0.11 mmol) was obtained as a yellow solid. LC-MS (ESI+): m/z 461 (MH⁺). ¹HNMR (300 MHz, CD₃OD) δ 8.90 (d, J=6.6 Hz, 2H), 8.42 (d, J=6.6 Hz, 2H), 7.03 (s, 1H), 6.88 (s, 1H), 4.25-4.10 (m, 4H), 3.96-3.85 (m, 4H), 3.69-3.64 (m, 2H), 3.30-3.17 (m, 3H), 2.93 (s, 3H), 2.48-2.39 (m, 2H), 2.16-2.02 (m, 2H).

### Example 8: Compound 35 Using General Synthetic Route 8

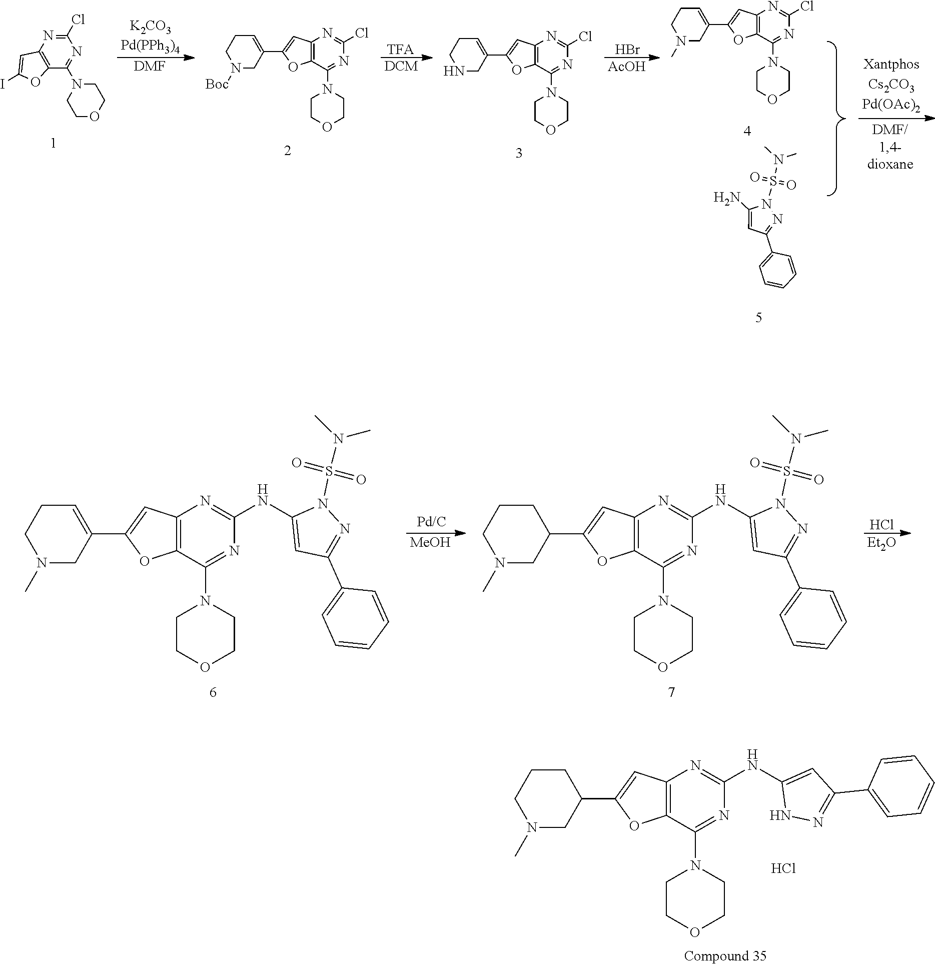

### 1.1) Synthesis of tert-butyl 3-(2-chloro-4-morpholinofuro[3,2-d]pyrimidin-6 yl)(5,6-dihydropyridine-1(2H)-carboxylate

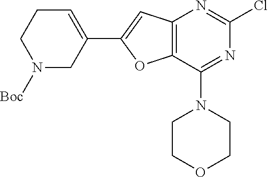 [SMILES: CC(C)(C)OC(=O)N1CCC=C(c2cc3nc(Cl)nc(N4CCOCC4)c3o2)C1]

A suspension of 2-chloro-6-iodo-4-morpholinofuro[3,2-d]pyrimidine (400 mg, 1.1 mmol), tert-butyl3-(4,4,5,5-tetramethyl-1,3,2-dioxaborolan-2-yl)-5,6-dihydropyridine-1(2H)-carboxylate (339 mg, 1.1 mmol), K₂CO₃ (454 mg, 3.29 mmol) and Pd(PPh₃)₄ (127 mg, 0.011 mmol) in 1,4-dioxane/H₂O (8/1, 40 mL) was heated to 90° C. for 3 h under N₂. The completion of the reaction was monitored by TLC. The reaction was diluted with water and extracted with EtOAc (3×30 mL). The combined organic phase was dried over anhydrous Na₂SO₄, filtrated and concentrated under reduced pressure. The residue was purified by silica gel column chromatography with a gradient elution of 10% EtOAc/PE to 33% EtOAc/PE to provide tert-butyl 3-(2-chloro-4-morpholinofuro[3,2-d]pyrimidin-6-yl)-5,6-dihydropyridine-1(2H)-carboxylate (155 mg, 0.37 mmol) as a yellow solid. LC-MS (ESI+): m/z 421/423 (MH⁺). ¹HNMR (300 MHz, CDCl₃) δ 6.68-6.62 (m, 1H), 6.55 (s, 1H), 4.25 (brs, 2H), 4.03-4.00 (m, 4H), 3.86-3.83 (m, 4H), 3.60-3.56 (m, 2H), 2.42-2.38 (m, 2H), 1.50 (s, 9H).

### 1.2) Synthesis of 2-chloro-4-morpholino-6-(1,2,5,6-tetrahydropyridin-3-yl)furo[3,2-d]pyrimidine

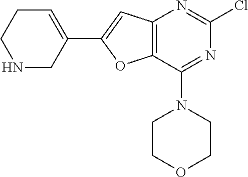 [SMILES: Clc1nc(N2CCOCC2)c2oc(C3=CCCNC3)cc2n1]

To a solution of tert-butyl3-(2-chloro-4-morpholinofuro[3,2-d]pyrimidin-6-yl)-5,6-dihydropyridine-1(2H)-carboxylate (600 mg, 1.43 mmol) in DCM (15 mL) was added TFA (3 mL). The reaction mixture was stirred at room temperature for 2 h. The completion of the reaction was monitored by TLC. The reaction was quenched with Na₂CO₃ and extracted with MeOH/DCM (1/15, 3×40 mL). The combined organic phase was dried over anhydrous Na₂SO₄, filtrated and concentrated under reduced pressure. The residue was slurried in MeOH/Et₂O (1/20, 10 mL) to provide 2-chloro-4-morpholino-6-(1,2,5,6-tetrahydropyridin-3-yl)furo[3,2-d]pyrimidine (385 mg, 1.20 mmol) as a white solid. LC-MS (ESI+): m/z 321/323 (MH⁺). ¹HNMR (300 MHz, CD₃OD) δ 6.94-6.91 (m, 1H), 6.82 (s, 1H), 4.25-4.05 (m, 6H), 3.90-3.83 (m, 4H), 3.59-3.54 (m, 2H), 2.87-2.70 (m, 2H).

### 1.3) Synthesis of 2-chloro-6-(1-methyl-1,2,5,6-tetrahydropyridin-3-yl)-4-morpholinofuro[3,2-d]pyrimidine

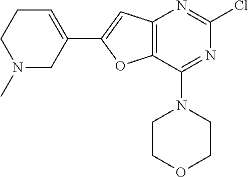 [SMILES: CN1CCC=C(c2cc3nc(Cl)nc(N4CCOCC4)c3o2)C1]

A solution of 2-chloro-4-morpholino-6-(1,2,5,6-tetrahydropyridin-3-yl)furo[3,2-d]pyrimidine (385 mg, 1.2 mmol), formaldehyde solution (488 mg, 37%, 6.02 mmol) and CH₃COOH (one drop) in DCM was stirred at room temperature for 30 min. To the solution was added sodium triacetoxyborohydride (1.27 g, 6.02 mmol). The reaction mixture was stirred at room temperature for 3 h. The completion of the reaction was monitored by TLC. The reaction mixture was quenched with a saturated NaHCO₃ solution and the pH was adjusted to 8. The aqueous solution was extracted with DCM (3×40 mL). The combined organic phase was dried over anhydrous Na₂SO₄, filtrated and concentrated under reduced pressure. The resulting residue was purified by silica gel column chromatography with a gradient elution of 1% MeOH/DCM to 3% MeOH/DCM to provide 2-chloro-6-(1-methyl-1,2,5,6-tetrahydropyridin-3-yl)-4-morpholinofuro[3,2-d]pyrimidine (375 mg, 1.12 mmol) as a brown solid. LC-MS (ESI+): m/z 335/337 (MH⁺). ¹HNMR (300 MHz, CDCl₃)⁶ 6.61-6.58 (m, 1H), 6.46 (s, 1H), 4.03-4.00 (m, 4H), 3.86-3.83 (m, 4H), 3.25-3.24 (m, 2H), 2.63-2.59 (m, 2H), 2.48-2.45 (m, 5H).

### 1.4) Synthesis of N,N-dimethyl-5-((6-(1-methyl-1,2,5,6-tetrahydropyridin-3-yl)-4-morpholinofuro[3,2-d]pyrimidin-2-yl)amino)-3-phenyl-1H-pyrazole-1-sulfonamide

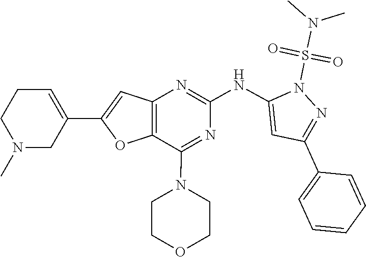 [SMILES: CN1CCC=C(c2cc3nc(Nc4cc(-c5ccccc5)nn4S(=O)(=O)N(C)C)nc(N4CCOCC4)c3o2)C1]

A suspension of 2-bromo-4-morpholino-6-(pyridin-4-yl)furo[3,2-d]pyrimidine (2×20 mg, 0.06 mmol), 5-amino-N,N-dimethyl-3-phenyl-1H-pyrazole-1-sulfonamide (19 mg, 0.07 mmol), Cs₂CO₃ (49 mg, 0.15 mmol), Pd(OAc)₂ (1 mg, 0.006 mmol) and Xantphos (3 mg, 0.006 mmol) in DMF/1,4-dioxane (1/7, 2 mL) was heated to 100° C. for 25 min under microwave conditions. The reaction mixture was concentrated directly and purified by silica gel column chromatography with a gradient elution of 1% MeOH/DCM to 2% MeOH/DCM to provide N,N-dimethyl-5-((6-(1-methyl-1,2,5,6-tetrahydropyridin-3-yl)-4-morpholinofuro[3,2-d]pyrimidin-2-yl)amino)-3-phenyl-1H-pyrazole-1-sulfonamide (50 mg, 0.089 mmol) as a brown solid. LC-MS (ESI+): m/z 565 (MH⁺). ¹HNMR (300 MHz, CDCl₃) δ 8.64 (s, 1H), 7.89 (d, J=6.6 Hz, 2H), 7.45-7.37 (m, 4H), 6.60-6.57 (m, 1H), 6.50 (s, 1H), 4.03-4.00 (m, 4H), 3.88-3.85 (m, 4H), 3.30-3.27 (m, 2H), 3.05 (s, 6H), 2.62-2.60 (m, 2H), 2.50-2.40 (m, 5H).

### 1.5) Synthesis of N,N-dimethyl-5-((6-(1-methylpiperidin-3-yl)-4-morpholinofuro [3,2-d]pyrimidin-2-yl)amino)-3-phenyl-1H-pyrazole-1-sulfonamide

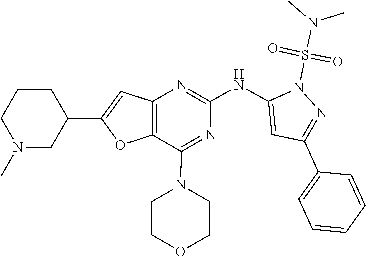 [SMILES: CN1CCCC(c2cc3nc(Nc4cc(-c5ccccc5)nn4S(=O)(=O)N(C)C)nc(N4CCOCC4)c3o2)C1]

To a solution of N,N-dimethyl-5-((6-(1-methyl-1,2,5,6-tetrahydropyridin-3-yl)-4-morpholinofuro [3,2-d]pyrimidin-2-yl)amino)-3-phenyl-1H-pyrazole-1-sulfonamide (50 mg, 0.089 mmol) in DCM/MeOH (1/1, 4 mL) was added Pd/C under H₂ (balloon). The reaction mixture was stirred at room temperature for 3 h. The completion of the reaction was monitored by LC-MS. After filtration, the filtrate was concentrated directly and purified by silica gel column chromatography with a gradient elution of 1% MeOH/DCM to 2% MeOH/DCM to provide N,N-dimethyl-5-((6-(1-methylpiperidin-3-yl)-4-morpholinofuro[3,2-d]pyrimidin-2-yl)amino)-3-phenyl-1H-pyrazole-1-sulfonamide (28 mg, 0.049 mmol) as a yellow solid. LC-MS (ESI+): m/z 567 (MH⁺). ¹HNMR (300 MHz, CDCl₃) δ 8.63 (s, 1H), 7.89 (dd, J=8.1, 1.5 Hz, 2H), 7.46-7.37 (m, 3H), 7.24 (s, 1H), 6.44 (s, 1H), 4.01-3.98 (m, 4H), 3.87-3.84 (m, 4H), 3.09-3.03 (m, 8H), 2.90-2.80 (m, 1H), 2.35 (s, 3H), 2.13-2.02 (m, 3H), 1.82-1.79 (m, 2H), 1.51-1.46 (m, 1H).

### 1.6) Synthesis of Compound 35, 6-(1-methylpiperidin-3-yl)-4-morpholino-N-(3-phenyl-1H-pyrazol-5-yl)furo[3,2-d]pyrimidin-2-amine hydrochloride

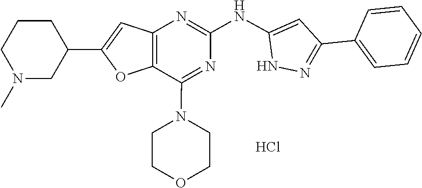 [SMILES: CN1CCCC(c2cc3nc(Nc4cc(-c5ccccc5)n[nH]4)nc(N4CCOCC4)c3o2)C1 ; Cl]

To a solution of N,N-dimethyl-5-((6-(1-methylpiperidin-3-yl)-4-morpholinofuro[3,2-d]pyrimidin-2-yl)amino)-3-phenyl-1H-pyrazole-1-sulfonamide (28 mg, 0.049 mmol) in DCM (4 mL) was added HCl/Et₂O (2 mL). The reaction mixture was stirred at room temperature for 2 h. After concentration and slurry in MeOH/Et₂O (1/20, 2 mL), 6-(1-methylpiperidin-3-yl)-4-morpholino-N-(3-phenyl-1H-pyrazol-5-yl)furo[3,2-d]pyrimidin-2-amine hydrochloride (Compound 35, 22 mg, 0.044 mmol) was obtained as a white solid. LC-MS (ESI+): m/z 460 (MH⁺). ¹HNMR (300 MHz, DMSO-d₆) δ 11.24 (s, 1H), 10.97 (s, 1H), 7.80 (d, J=7.5 Hz, 2H), 7.52-7.47 (m, 2H), 7.43-7.38 (m, 1H), 6.93 (s, 1H), 6.60 (s, 1H), 4.20-4.08 (m, 4H), 3.90-3.82 (m, 4H), 3.72-3.61 (m, 1H), 3.53-3.39 (m, 2H), 3.22-3.14 (m, 1H), 3.06-2.94 (m, 1H), 2.78 (s, 3H), 2.19-2.08 (m, 1H), 2.03-1.97 (m, 2H), 1.69-1.59 (m, 1H).

### Example 9: Compound 39 Using General Synthetic Route 9

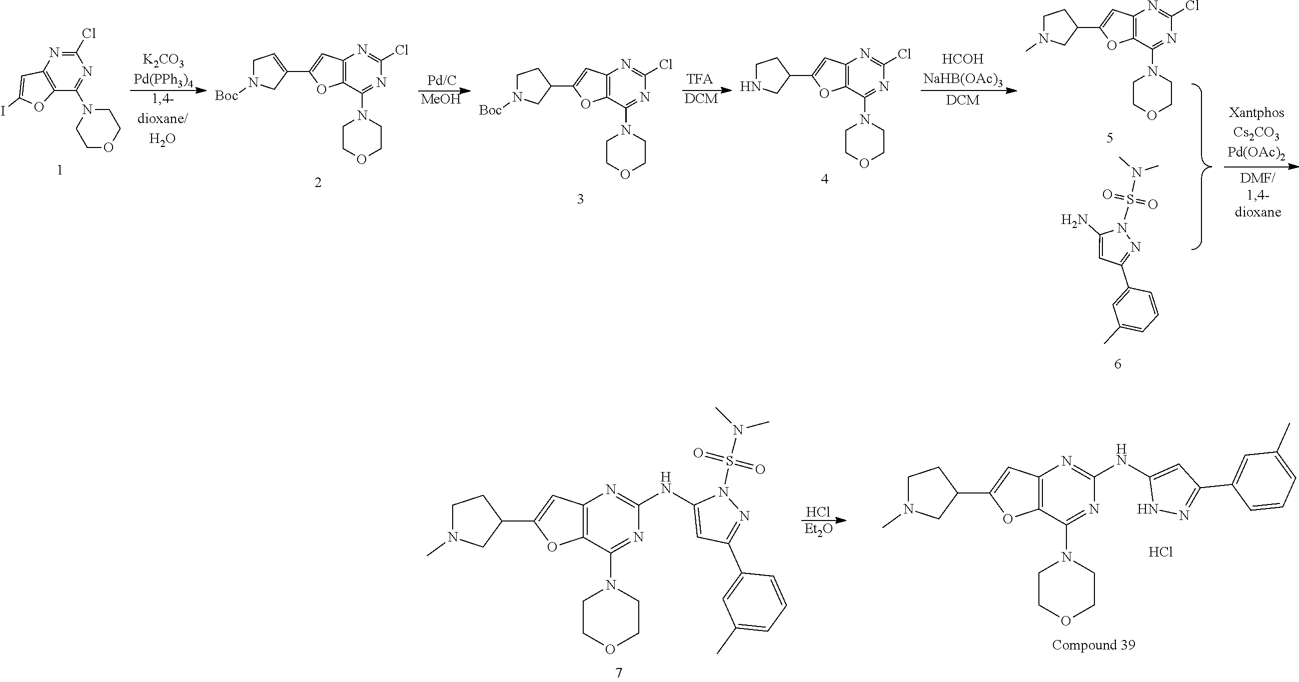

### 1.1) Synthesis of tert-butyl 3-(2-chloro-4-morpholinofuro[3,2-d]pyrimidin-6-yl)-2,5-dihydro-1H-pyrrole-1-carboxylate

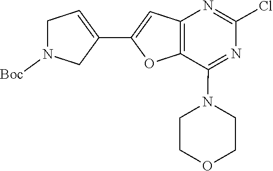 [SMILES: CC(C)(C)OC(=O)N1CC=C(c2cc3nc(Cl)nc(N4CCOCC4)c3o2)C1]

A suspension of 2-chloro-6-iodo-4-morpholinofuro[3,2-d]pyrimidine (800 mg, 2.2 mmol), tert-butyl 3-(4,4,5,5-tetramethyl-1,3,2-dioxaborolan-2-yl)-2,5-dihydro-1H-pyrrole-1-carboxylate (649 mg, 2.2 mmol), K₂CO₃ (911 mg, 6.6 mmol) and Pd(PPh₃)₄ (254 mg, 0.022 mmol) in 1,4-dioxane/H₂O (2/1, 60 mL) was heated to 90° C. for 3 h under N₂. The completion of the reaction was monitored by TLC. The reaction was diluted with water and extracted with EtOAc (3×30 mL). The combined organic phase was dried over anhydrous Na₂SO₄, filtrated and concentrated under reduced pressure. The residue was purified by silica gel column chromatography with a gradient elution of 10% EtOAc/PE to 50% EtOAc/PE to provide tert-butyl 3-(2-chloro-4-morpholinofuro[3,2-d]pyrimidin-6-yl)-2,5-dihydro-1H-pyrrole-1-carboxylate (400 mg, 0.99 mmol) as a yellow solid. LC-MS (ESI+): m/z 407/409 (MH⁺). ¹HNMR (300 MHz, CDCl₃) δ 6.60 (s, 0.51H), 6.50 (s, 0.51H), 6.40 (s, 0.51H), 6.35 (s, 0.5H), 4.53-4.35 (m, 4H), 4.05-4.01 (m, 4H), 3.86-3.82 (m, 4H), 1.51 (s, 9H).

### 1.2) Synthesis of tert-butyl 3-(2-chloro-4-morpholinofuro[3,2-d]pyrimidin-6-yl)pyrrolidine-1-carboxylate

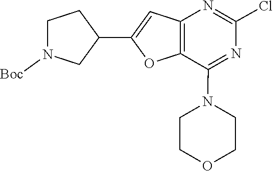 [SMILES: CC(C)(C)OC(=O)N1CCC(c2cc3nc(Cl)nc(N4CCOCC4)c3o2)C1]

To a solution of tert-butyl 3-(2-chloro-4-morpholinofuro[3,2-d]pyrimidin-6-yl)-2,5-dihydro-1H-pyrrole-1-carboxylate (50 mg, 0.089 mmol) in DCM/MeOH (1/1, 20 mL) was added Pd/C under H₂ (balloon). The reaction mixture was stirred at room temperature overnight. The completion of the reaction was monitored by LC-MS. After filtration, the filtrate was concentrated directly and purified by silica gel column chromatography with a gradient elution of 10% EtOAc/PE to 33% EtOAc/PE to provide tert-butyl 3-(2-chloro-4-morpholinofuro[3,2-d]pyrimidin-6-yl)pyrrolidine-1-carboxylate (300 mg, 0.74 mmol) as a white solid. LC-MS (ESI+): m/z 409/411 (MH⁺).

### 1.3) Synthesis of 2-chloro-4-morpholino-6-(pyrrolidin-3-yl)furo[3,2-d]pyrimidine

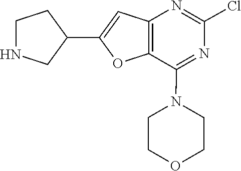 [SMILES: Clc1nc(N2CCOCC2)c2oc(C3CCNC3)cc2n1]

To a solution of tert-butyl 3-(2-chloro-4-morpholinofuro[3,2-d]pyrimidin-6-yl)-5,6-dihydropyridine-1(2H)-carboxylate (300 mg, 0.74 mmol) in DCM (15 mL) was added TFA (3 mL). The reaction mixture was stirred at room temperature for 2 h. The completion of the reaction was monitored by TLC. The solution was quenched with Na₂CO₃ and the pH was adjusted to 8. A large amount of solid was precipitated. After filtration, 2-chloro-4-morpholino-6-(pyrrolidin-3-yl)furo[3,2-d]pyrimidine (200 mg, 0.65 mmol) was obtained as a white solid. LC-MS (ESI+): m/z 309/311 (MH⁺).

### 1.4) Synthesis of 2-chloro-6-(1-methylpyrrolidin-3-yl)-4-morpholinofuro[3,2-d]pyrimidine

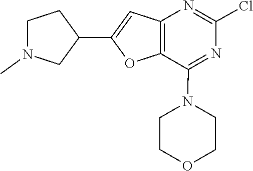 [SMILES: CN1CCC(c2cc3nc(Cl)nc(N4CCOCC4)c3o2)C1]

A solution of 2-chloro-4-morpholino-6-(pyrrolidin-3-yl)furo[3,2-d]pyrimidine (200 mg, 0.65 mmol), formaldehyde solution (264 mg, 37%, 3.25 mmol) and CH₃COOH (one drop) in DCM was stirred at room temperature for 30 min. Then to the reaction was added sodium triacetoxyborohydride (690 mg, 6.02 mmol). The reaction mixture was stirred at room temperature for 3 h. The completion of the reaction was monitored by TLC. The reaction mixture was quenched with a saturated NaHCO₃ solution and the pH was adjusted to 8. The aqueous solution was extracted with DCM (3×40 mL). The combined organic phase was dried over anhydrous Na₂SO₄, filtrated and concentrated under reduced pressure. The resulting residue was purified by silica gel column chromatography with a gradient elution of 1% MeOH/DCM to 5% MeOH/DCM to provide 2-chloro-6-(1-methylpyrrolidin-3-yl)-4-morpholinofuro[3,2-d]pyrimidine (180 mg, 0.56 mmol) as a white solid. LC-MS (ESI+): m/z 323/325 (MH⁺). ¹HNMR (300 MHz, CDCl₃) δ6.80 (s, 1H), 3.92-3.89 (m, 4H), 3.75-3.74 (m, 4H), 3.69-3.66 (m, 1H), 3.12-3.05 (m, 1H), 2.90-2.81 (m, 3H), 2.50 (s, 3H), 2.37-2.30 (m, 1H), 2.09-2.01 (m, 1H).

### 1.5) Synthesis of N,N-dimethyl-5-((6-(1-methylpyrrolidin-3-yl)-4-morpholinofuro[3,2-d]pyrimidin-2-yl)amino)-3-(m-tolyl)-1H-pyrazole-1-sulfonamide

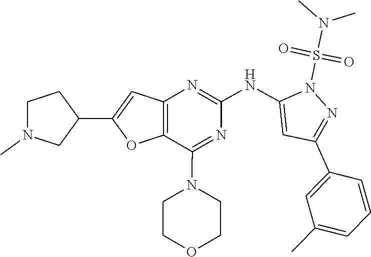 [SMILES: Cc1cccc(-c2cc(Nc3nc(N4CCOCC4)c4oc(C5CCN(C)C5)cc4n3)n(S(=O)(=O)N(C)C)n2)c1]

A suspension of 2-chloro-6-(1-methylpyrrolidin-3-yl)-4-morpholinofuro[3,2-d]pyrimidine (40 mg, 0.12 mmol), 5-amino-N,N-dimethyl-3-(m-tolyl)-1H-pyrazole-1-sulfonamide (45 mg, 0.16 mmol), Cs₂CO₃ (100 mg, 0.31 mmol), Pd(OAc)₂ (3 mg, 0.012 mmol) and Xantphos (7 mg, 0.012 mmol) in DMF/1,4-dioxane (1/7, 5 mL) was heated to 90° C. for 30 min under microwave condition. The reaction mixture was concentrated directly and purified by silica gel column chromatography with a gradient elution of 1% MeOH/DCM to 5% MeOH/DCM to provide N,N-dimethyl-5-((6-(1-methylpyrrolidin-3-yl)-4-morpholinofuro[3,2-d]pyrimidin-2-yl)amino)-3-(m-tolyl)-1H-pyrazole-1-sulfonamide (60 mg, 0.11 mmol). LC-MS (ESI+): m/z 567 (MH⁺). ¹HNMR (300 MHz, CD₃OD) δ7.67-7.62 (m, 2H), 7.35-7.30 (m, 1H), 7.23-7.21 (m, 1H), 7.16 (s, 1H), 6.56 (s, 1H), 4.04-4.01 (m, 4H), 3.87-3.84 (m, 4H), 3.69-3.54 (m, 1H), 3.15-3.05 (m, 2H), 2.90 (s, 6H), 2.85-2.75 (m, 2H), 2.46 (s, 3H), 2.41 (s, 3H), 2.37-2.30 (m, 1H), 2.19-2.10 (m, 1H).

### 1.6) Synthesis of Compound 39, 6-(1-methylpyrrolidin-3-yl)-4-morpholino-N-(3-(m-tolyl)-1H-pyrazol-5-yl)furo[3,2-d]pyrimidin-2-amine hydrochloride

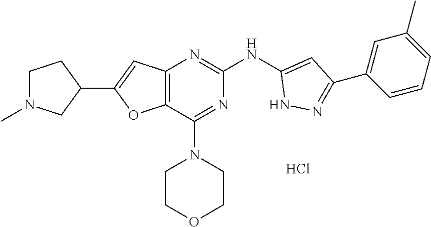 [SMILES: Cc1cccc(-c2cc(Nc3nc(N4CCOCC4)c4oc(C5CCN(C)C5)cc4n3)[nH]n2)c1 ; Cl]

To a solution of N,N-dimethyl-5-((6-(1-methylpyrrolidin-3-yl)-4-morpholinofuro[3,2-d]pyrimidin-2-yl)amino)-3-(m-tolyl)-1H-pyrazole-1-sulfonamide (60 mg, 0.11 mmol) in DCM (4 mL) was added HCl/Et₂O (2 mL). The reaction mixture was stirred at room temperature for 2 h. After concentration, the crude product was purified by preparative HPLC to provide 6-(1-methylpyrrolidin-3-yl)-4-morpholino-N-(3-(m-tolyl)-1H-pyrazol-5-yl)furo[3,2-d]pyrimidin-2-amine hydrochloride (Compound 39, 9 mg, 0.018 mmol) as a yellow solid. LC-MS (ESI+): m/z 460 (MH⁺). ¹HNMR (300 MHz, D₂O) δ 7.17-7.04 (m, 4H), 6.64 (d, J=3.0 Hz, 1H), 6.03 (s, 1H), 6.41 (s, 1H), 3.99-3.94 (m, 1H), 3.92-3.76 (m, 8H), 3.75-3.68 (m, 1H), 3.66-3.42 (m, 1H), 3.28-3.09 (m, 2H), 2.92 (d, J=5.1 Hz, 3H), 2.64-2.44 (m, 1H), 2.32-2.22 (m, 1H), 2.17 (s, 3H).

### Example 10: Compound 42 Using General Synthetic Route 10

### General Procedure 10:

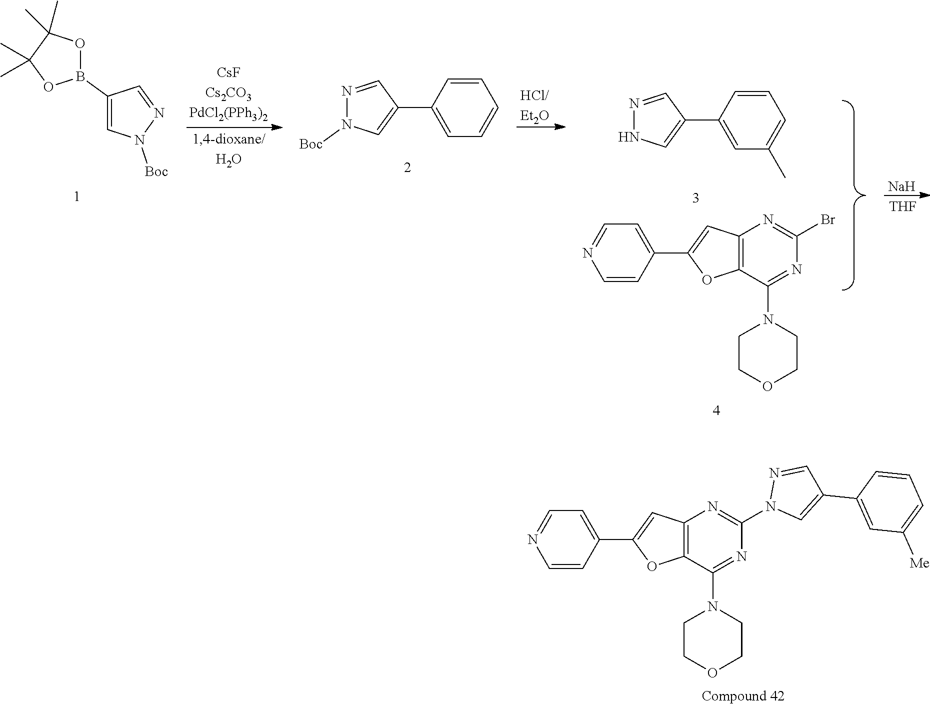

### 1.1) Synthesis of tert-butyl 4-(m-tolyl)-1H-pyrazole-1-carboxylate

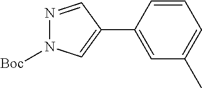 [SMILES: Cc1cccc(-c2cnn(C(=O)OC(C)(C)C)c2)c1]

A solution of tert-butyl 4-(4,4,5,5-tetramethyl-1,3,2-dioxaborolan-2-yl)-1H-pyrazole-1-carboxylate (1.5 g, 5.1 mmol) 1-bromo-3-methylbenzene (872 mg, 5.1 mmol), Cs₂CO₃ (91 mg, 0.28 mmol), PdCl₂(PPh₃)₂ (590 mg, 0.051 mmol) and CsF (1.16 g, 7.65 mmol) in 1,4-dioxane/H₂O (2/1, 30 mL) was heated to 80° C. overnight under N₂. The completion of the reaction was monitored by TLC. The reaction mixture was concentrated directly and purified by silica gel column chromatography with a gradient elution of 1% MeOH/DCM to 5% MeOH/DCM to provide tert-butyl 4-(m-tolyl)-1H-pyrazole-1-carboxylate (300 mg, 1.16 mmol) as a yellow solid. LC-MS (ESI+): m/z 259 (MH⁺). ¹HNMR (300 MHz, CDCl₃) δ 8.29 (s, 1H), 7.99 (s, 1H), 7.34-7.28 (m, 3H), 7.13-7.10 (m, 1H), 2.39 (s, 3H), 1.68 (s, 9H).

### Synthesis of 4-(m-tolyl)-1H-pyrazole hydrochloride

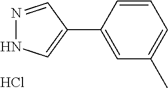

To a solution of tert-butyl 4-(m-tolyl)-1H-pyrazole-1-carboxylate (300 mg, 1.16 mmol) in DCM (8 mL) was added HCl/Et₂O (3 mL). The reaction mixture was stirred at room temperature for 2 h. A large amount of solid was precipitated. After concentration and slurry in MeOH/Et₂O (1/20, 2 mL), 4-(m-tolyl)-1H-pyrazole hydrochloride (153 mg, 0.79 mmol) was obtained as a yellow solid. LC-MS (ESI+): m/z 159 (MH⁺). ¹HNMR (300 MHz, CDCl₃) δ 8.13 (s, 2H), 7.38-7.20 (m, 4H), 2.42 (s, 3H).

### 1.2) Synthesis of Compound 42, 4-morpholino-6-(pyridin-4-yl)-2-(4-(m-tolyl)-1H-pyrazol-1-yl)furo[3,2-d]pyrimidine

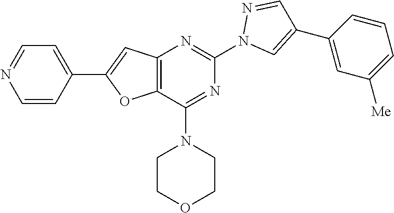 [SMILES: Cc1cccc(-c2cnn(-c3nc(N4CCOCC4)c4oc(-c5ccncc5)cc4n3)c2)c1]

A suspension of 2-bromo-4-morpholino-6-(pyridin-4-yl)furo[3,2-d]pyrimidine (60 mg, 0.17 mmol) and 4-(m-tolyl)-1H-pyrazole hydrochloride (40 mg, 0.20 mmol) in anhydrous THE (10 mL) was added NaH (12 mg, 0.49 mmol). The reaction mixture was stirred at 70° C. overnight. The completion was monitored by TLC. The reaction was quenched with water (10 mL) and the aqueous solution was extracted with DCM/MeOH (10/1, 2×20 mL). The combined organic phase was dried over anhydrous Na₂SO₄, filtrated and concentrated under reduced pressure. The residue was purified by preparative TLC purification to provide 4-morpholino-6-(pyridin-4-yl)-2-(4-(m-tolyl)-1H-pyrazol-1-yl)furo[3,2-d]pyrimidine (Compound 42, 31.2 mg, 0.049 mmol) as a yellow solid. LC-MS (ESI+): m/z 439 (MH⁺). ¹HNMR (300 MHz, CDCl₃) δ 8.80-8.78 (m, 2H), 8.71 (s, 1H), 8.08 (s, 1H), 7.68 (d, J=5.7 Hz, 2H), 7.42 (d, J=7.5 Hz, 2H), 7.34-7.31 (m, 2H), 7.11 (d, J=3.9 Hz, 1H), 4.22-4.16 (m, 4H), 3.98-3.91 (m, 4H), 2.41 (s, 3H).

### Example 11: Compound 43 Using General Synthetic Route 11

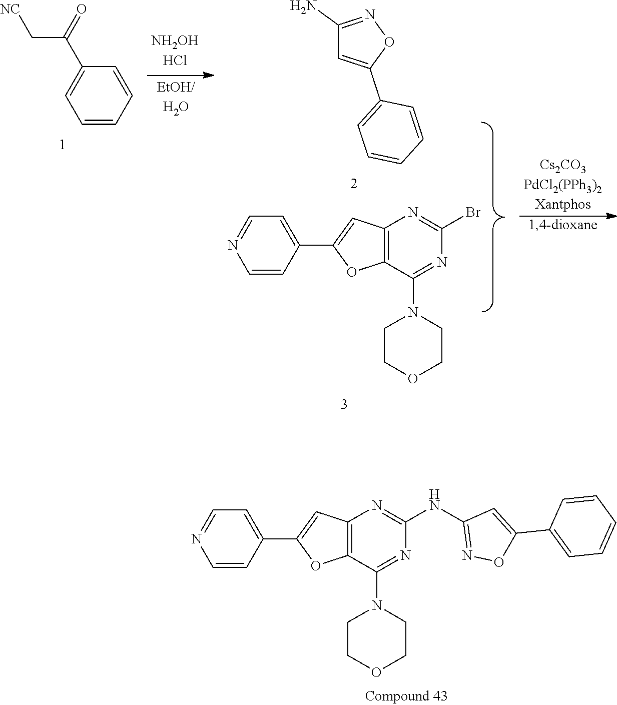 [SMILES: N#CCC(=O)c1ccccc1 ; Nc1cc(-c2ccccc2)on1 ; "Cs2CO3PdCl2(PPh3)2Xantphos" ; Brc1nc(N2CCOCC2)c2oc(-c3ccncc3)cc2n1 ; "1,4-dioxane" ; c1ccc(-c2cc(Nc3nc(N4CCOCC4)c4oc(-c5ccncc5)cc4n3)no2)cc1]

### 1.1) Synthesis of 5-phenylisoxazol-3-amine

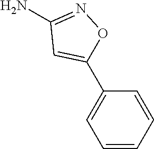 [SMILES: Nc1cc(-c2ccccc2)on1]

A solution of 3-oxo-3-phenylpropanenitrile (1.5 g, 10.3 mmol) in EtOH/H₂O (1/1, 20 mL) was added hydroxylamine hydrochloride (785 mg, 11.3 mmol) and sodium hydroxide (450 mg, 11.3 mmol). The reaction mixture was heated to 80° C. overnight. To the above solution was added conc. HCl aq. (1.3 mL). The resulting mixture was stirred at 80° C. for 2 h. The completion of the reaction was monitored by TLC. The reaction mixture was quenched with a saturated NaHCO₃ solution and the pH was adjusted to 10. The aqueous solution was extracted with EtOAc (3×20 mL). The combined organic phase was dried over anhydrous Na₂SO₄, filtrated and concentrated under reduced pressure. The residue was purified by silica gel column chromatography with a gradient elution of 15% EtOAc/hex to 35% EtOAc/hex to provide 5-phenylisoxazol-3-amine (0.68 g, 1.25 mmol) as a yellow solid. LC-MS (ESI+): m/z 161 (MH⁺). ¹HNMR (300 MHz, CDCl₃) δ 7.72-7.68 (m, 2H), 7.47-7.35 (m, 3H), 6.08 (s, 1H), 4.08 (brs, 2H).

### 1.2) Synthesis of Compound 43, 4-morpholino-N-(5-phenylisoxazol-3-yl)-6-(pyridin-4-yl) furo[3,2-d]pyrimidin-2-amine

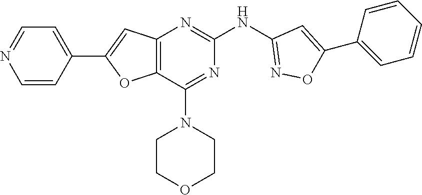 [SMILES: c1ccc(-c2cc(Nc3nc(N4CCOCC4)c4oc(-c5ccncc5)cc4n3)no2)cc1]

A suspension of 2-bromo-4-morpholino-6-(pyridin-4-yl)furo[3,2-d]pyrimidine (50 mg, 0.14 mmol), 5-phenylisoxazol-3-amine (35 mg, 0.21 mmol), Cs₂CO₃ (91 mg, 0.28 mmol), PdCl₂(PPh₃)₂ (10 mg, 0.014 mmol) and Xantphos (24 mg, 0.042 mmol) in 1,4-dioxane (4 mL) was heated to 90° C. for 30 min under microwave condition. The reaction mixture was concentrated directly and purified by silica gel column chromatography with a gradient elution of 1% MeOH/DCM to 5% MeOH/DCM to provide 4-morpholino-N-(5-phenylisoxazol-3-yl)-6-(pyridin-4-yl)furo[3,2-d]pyrimidin-2-amine (Compound 43, 15 mg, 0.034 mmol) as a white solid. LC-MS (ESI+): m/z 441 (MH⁺). ¹HNMR (300 MHz, DMSO-d₆) δ 9.93 (s, 1H), 8.74 (d, J=6.0 Hz, 2H), 7.93 (d, J=6.0 Hz, 2H), 7.83-7.81 (m, 2H), 7.69 (s, 1H), 7.56-7.53 (m, 3H), 7.40 (s, 1H), 4.08-4.02 (m, 4H), 3.85-3.79 (m, 4H).

### Example 12: Compound 44 Using General Synthetic Route 12

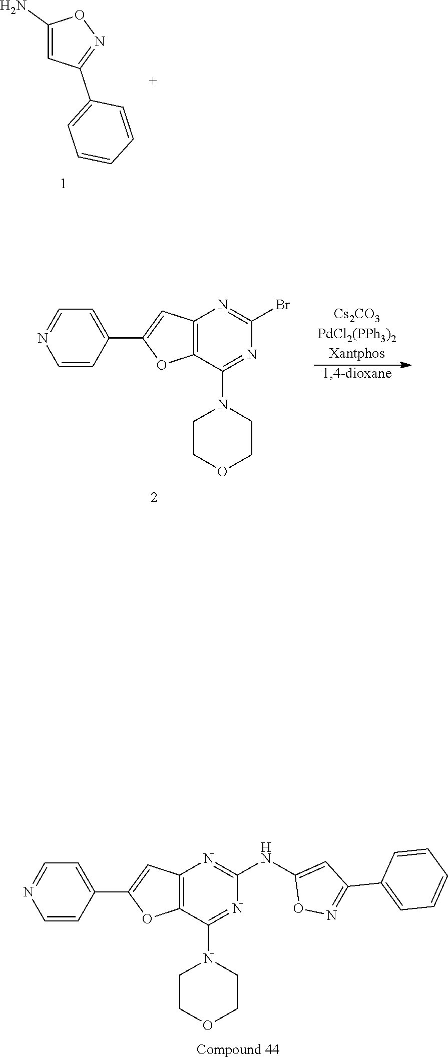 [SMILES: Nc1cc(-c2ccccc2)no1 ; Brc1nc(N2CCOCC2)c2oc(-c3ccncc3)cc2n1 ; "1,4-dioxane" ; "Cs2CO3PdCl2(PPh3)2Xantphos" ; c1ccc(-c2cc(Nc3nc(N4CCOCC4)c4oc(-c5ccncc5)cc4n3)on2)cc1]

### 1.1) Synthesis of Compound 44, 4-morpholino-N-(3-phenylisoxazol-5-yl)-6-(pyridin-4-yl) furo[3,2-d]pyrimidin-2-amine

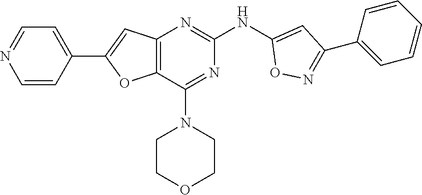 [SMILES: c1ccc(-c2cc(Nc3nc(N4CCOCC4)c4oc(-c5ccncc5)cc4n3)on2)cc1]

A suspension of 2-bromo-4-morpholino-6-(pyridin-4-yl)furo[3,2-d]pyrimidine (76 mg, 0.21 mmol), 3-phenylisoxazol-5-amine (40 mg, 0.25 mmol), Cs₂CO₃ (158 mg, 0.48 mmol), Pd(OAc)₂ (5 mg, 0.021 mmol) and Xantphos (12 mg, 0.021 mmol) in DMF/1,4-dioxane (1/7, 8 mL) was heated to 80° C. for 25 min under microwave condition. The reaction mixture was concentrated directly and purified by silica gel column chromatography with a gradient elution of 1% MeOH/DCM to 5% MeOH/DCM to provide 4-morpholino-N-(3-phenylisoxazol-5-yl)-6-(pyridin-4-yl)furo[3,2-d]pyrimidin-2-amine (Compound 44, 61 mg, 0.14 mmol) as a yellow solid. LC-MS (ESI+): m/z 441 (MH⁺). ¹HNMR (300 MHz, DMSO-d₆) δ 10.86 (s, 1H), 8.75 (d, J=4.5 Hz, 2H), 7.93 (d, J=5.7 Hz, 2H), 7.84-7.81 (m, 2H), 7.75 (s, 1H), 7.53-7.51 (m, 3H), 6.70 (s, 1H), 4.08-4.03 (m, 4H), 3.87-3.82 (m, 4H).

### Example 13: Compound 50 Using General Synthetic Route 13

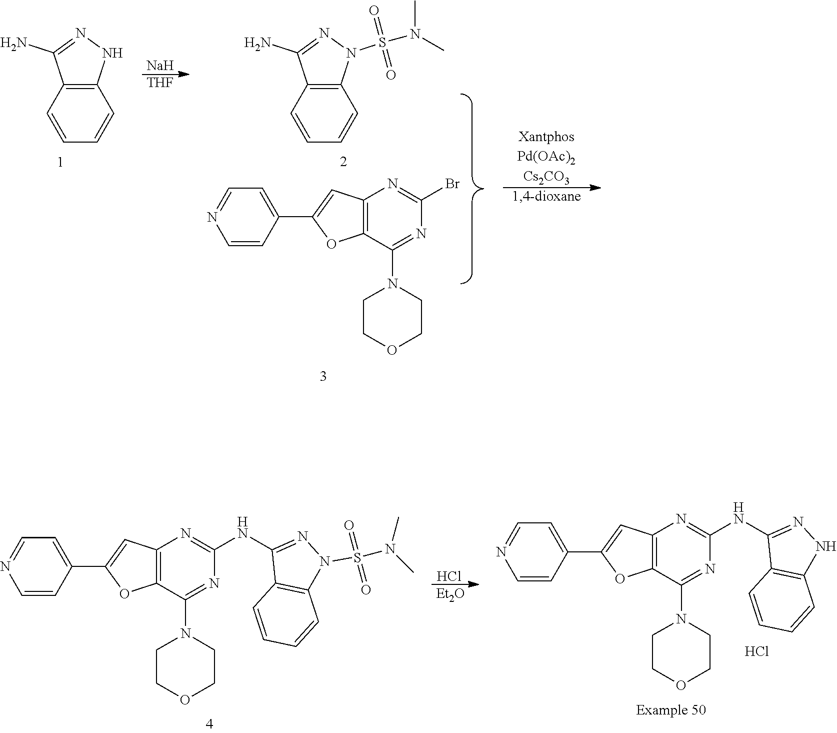

### 1.1) Synthesis of 3-amino-N, N-dimethyl-H-indazole-1-sulfonamide

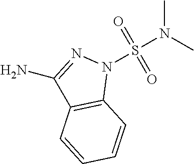 [SMILES: CN(C)S(=O)(=O)n1nc(N)c2ccccc21]

To a solution of 1H-indazol-3-amine (1 g, 7.5 mmol) in THF (30 mL) at 0° C. was added NaH (541 mg, 13.53 mmol). After stirred at 0° C. for 1 h, to the solution was added dimethylsulfamoyl chloride (1.61 g, 11.28 mmol). The completion of the reaction was monitored by TLC. The reaction mixture was quenched with a saturated NH₄Cl solution. The aqueous solution was extracted with DCM/MeOH (15/1, 3×50 mL). The combined organic phase was dried over anhydrous Na₂SO₄, filtrated and concentrated under reduced pressure. The resulting residue was purified by silica gel column chromatography with a gradient elution of 20% EtOAc/PE to 50% EtOAc/PE to provide 3-amino-N,N-dimethyl-1H-indazole-1-sulfonamide (600 mg, 2.5 mmol) as a yellow solid. LC-MS: m/z 241 (MH⁺). ¹HNMR (300 MHz, CDCl₃) δ 7.97 (d, J=8.4 Hz, 1H), 7.55-7.48 (m, 2H), 7.29-7.24 (m, 1H), 4.46 (brs, 2H), 2.92 (s, 6H).

### 1.2) Synthesis of N,N-dimethyl-3-((4-morpholino-6-(pyridin-4-yl)furo[3,2-d]pyrimidin-2-yl) amino)-1H-indazole-1-sulfonamide

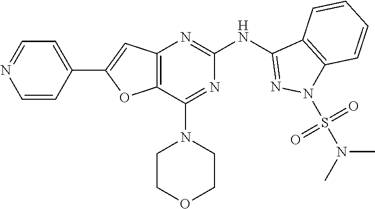 [SMILES: CN(C)S(=O)(=O)n1nc(Nc2nc(N3CCOCC3)c3oc(-c4ccncc4)cc3n2)c2ccccc21]

A suspension of 2-bromo-4-morpholino-6-(pyridin-4-yl)furo[3,2-d]pyrimidine (60 mg, 0.17 mmol), 3-amino-N,N-dimethyl-1H-indazole-1-sulfonamide (48 mg, 0.20 mmol), Cs₂CO₃ (125 mg, 0.38 mmol), Pd(OAc)₂ (4 mg, 0.017 mmol) and Xantphos (10 mg, 0.017 mmol) in DMF/1,4-dioxane (1/7, 4 mL) was heated to 90° C. for 25 min under microwave condition. The reaction mixture was concentrated directly and purified by silica gel column chromatography with a gradient elution of 1% MeOH/DCM to 5% MeOH/DCM to provide N,N-dimethyl-3-((4-morpholino-6-(pyridin-4-yl)furo[3,2-d]pyrimidin-2-yl)amino)-1H-indazole-1-sulfonamide (50 mg, 0.096 mmol) as a white solid. LC-MS (ESI+): m/z 521 (MH⁺). ¹HNMR (300 MHz, CDCl₃) δ 8.74 (d, J=6.0 Hz, 2H), 8.04 (d, J=8.7 Hz, 1H), 7.9 (d, J=8.4 Hz, 1H), 7.65 (d, J=6.0 Hz, 2H), 7.60-7.48 (m, 2H), 7.16 (s, 1H), 4.02-3.97 (m, 4H), 3.83-3.80 (m, 4H), 2.99 (s, 6H).

### 1.3) Synthesis of Compound 50, N-(1H-indazol-3-yl)-4-morpholino-6-(pyridin-4-yl)furo[3,2-d]pyrimidin-2-amine hydrochloride

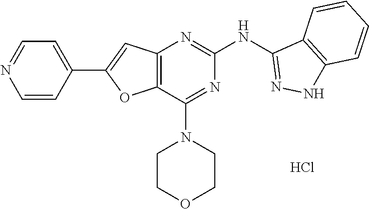 [SMILES: c1ccc2c(Nc3nc(N4CCOCC4)c4oc(-c5ccncc5)cc4n3)n[nH]c2c1 ; Cl]

To a solution of N,N-dimethyl-3-((4-morpholino-6-(pyridin-4-yl)furo[3,2-d]pyrimidin-2-yl)amino)-1H-indazole-1-sulfonamide (50 mg, 0.096 mmol) in DCM (4 mL) was added HCl/Et₂O (2 mL). The reaction mixture was stirred at room temperature for 2 h. After concentration and slurry in MeOH/Et₂O (1/20, 2 mL), N-(1H-indazol-3-yl)-4-morpholino-6-(pyridin-4-yl)furo[3,2-d]pyrimidin-2-amine hydrochloride (Compound 50, 33.8 mg, 0.06 mmol) was obtained as a yellow solid. LC-MS (ESI+): m/z 414 (MH⁺). ¹HNMR (300 MHz, CD₃OD) δ 8.94 (d, J=6.6 Hz, 2H), 8.48 (d, J=5.7 Hz, 2H), 7.99 (d, J=9.0 Hz, 2H), 7.58-7.48 (m, 2H), 7.23 (t, J=7.5 Hz, 1H), 4.44-4.15 (m, 4H), 3.96-3.87 (m, 4H).

### Example 14: Compound 52 Using General Synthetic Route 14

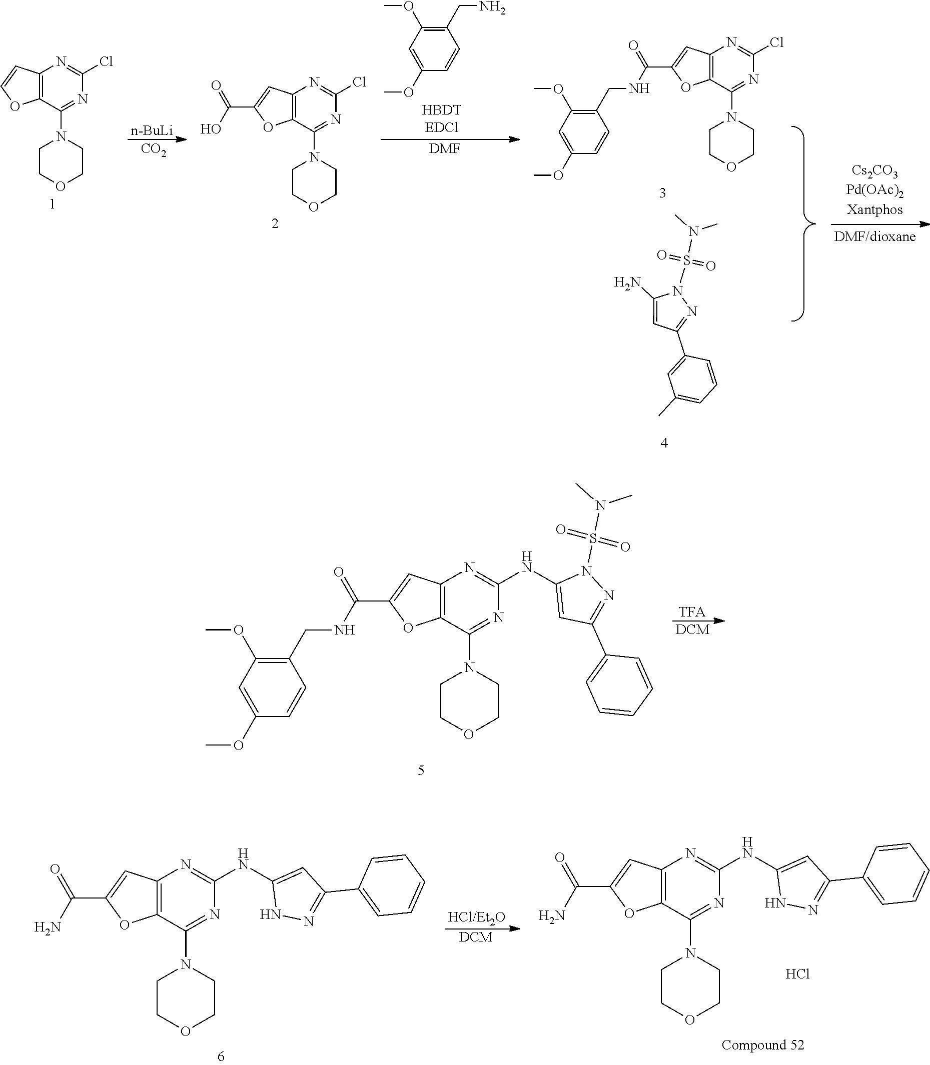

### 1.1) Synthesis of 2-chloro-4-morpholinofuro[3,2-d]pyrimidine-6-carboxylic acid

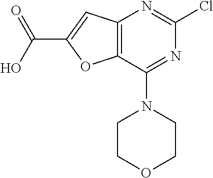 [SMILES: O=C(O)c1cc2nc(Cl)nc(N3CCOCC3)c2o1]

To a solution of 2-chloro-4-morpholinofuro[3,2-d]pyrimidine (2.4 g, 10 mmol) in anhydrous TH (4 mL) at −78° C. under N₂ was added n-BuLi (5.2 mL, 2.5 M, 13 mmol) dropwise. The reaction mixture was stirred at that temperature for 1 h. To the solution was added dry ice (4.4 g, 100 mmol) in one portion. The resulting reaction mixture was stirred at that temperature for 3 h. The completion of the reaction was monitored by TLC. The reaction was quenched with water and the pH was adjusted to 5. The aqueous solution was extracted with DCM (3×80 mL). The combined organic phase was dried over anhydrous Na₂SO₄, filtrated and concentrated under reduced pressure. The resulting residue was slurry in Et₂O to provide 2-chloro-4-morpholinofuro[3,2-d]pyrimidine-6-carboxylic acid (2.92 g, 10.3 mmol) as a yellow solid. LC-MS (ESI+): m/z 284/286 (MH⁺) ¹HNMR (300 MHz, CDCl₃) δ 7.58 (s, 1H), 4.04-3.95 (m, 4H), 3.81-3.76 (m, 4H).

### 1.2) Synthesis of 2-chloro-N-(2,4-dimethoxybenzyl)-4-morpholinofuro[3,2-d]pyrimidine-6-carboxamide

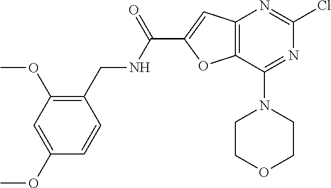 [SMILES: COc1ccc(CNC(=O)c2cc3nc(Cl)nc(N4CCOCC4)c3o2)c(OC)c1]

A solution of 2-chloro-4-morpholinofuro[3,2-d]pyrimidine-6-carboxylic acid (112 mg, 0.41 mmol), (2,4-dimethoxyphenyl)methanamine (68 mg, 0.41 mmol), HBOT (137 mg, 1.02 mmol) and EDCl (195 mg, 1.02 mmol) in DMF was stirred at room temperature overnight. The completion of the reaction was monitored by TLC. The solution was diluted with water (10 mL) and extracted with DCM/MeOH (10/1, 3×30 mL). The combined organic phase was dried over anhydrous Na₂SO₄, filtrated and concentrated under reduced pressure to provide crude 2-chloro-N-(2,4-dimethoxybenzyl)-4-morpholinofuro[3,2-d]pyrimidine-6-carboxamide (140 mg, 0.32 mmol) as a yellow oil. LC-MS (ESI+): m/z 433/435 (MH⁺).

### 1.3) Synthesis of N-(2,4-dimethoxybenzyl)-2-((1-(N,N-dimethylsulfamoyl)-3-phenyl-1H-pyrazol-5-yl)amino)-4-morpholinofuro[3,2-d]pyrimidine-6-carboxamide

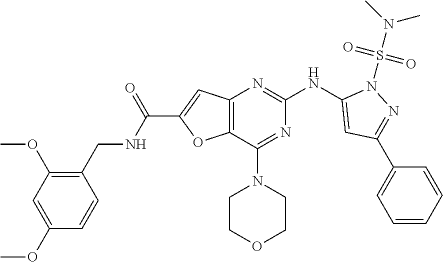 [SMILES: COc1ccc(CNC(=O)c2cc3nc(Nc4cc(-c5ccccc5)nn4S(=O)(=O)N(C)C)nc(N4CCOCC4)c3o2)c(OC)c1]

A suspension of 2-chloro-N-(2,4-dimethoxybenzyl)-4-morpholinofuro[3,2-d]pyrimidine-6-carboxamide (2×50 mg, 0.12 mmol), 5-amino-N,N-dimethyl-3-phenyl-1H-pyrazole-1-sulfonamide (37 mg, 0.14 mmol), Cs₂CO₃ (90 mg, 0.27 mmol), Pd(OAc)₂ (3 mg, 0.012 mmol) and Xantphos (6.5 mg, 0.012 mmol) in DMF/1,4-dioxane (1/7, 4 mL) was heated to 90° C. for 30 min under microwave conditions. The reaction mixture was concentrated directly and purified by silica gel column chromatography with a gradient elution of 1% MeOH/DCM to 5% MeOH/DCM to provide N-(2,4-dimethoxybenzyl)-2-((1-(N,N-dimethylsulfamoyl)-3-phenyl-1H-pyrazol-5-yl)amino)-4-morpholinofuro[3,2-d]pyrimidine-6-carboxamide (43 mg, 0.065 mmol) as a yellow solid. LC-MS (ESI+): m/z 663 (MH⁺).

### 1.4) Synthesis of Compound 52, 4-morpholino-2-((3-phenyl-1H-pyrazol-5-yl)amino)furo[3,2-d]pyrimidine-6-carboxamide

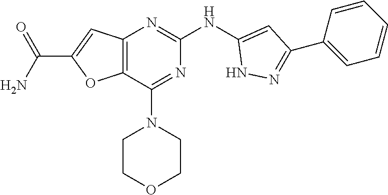 [SMILES: NC(=O)c1cc2nc(Nc3cc(-c4ccccc4)n[nH]3)nc(N3CCOCC3)c2o1]

To a solution of N-(2,4-dimethoxybenzyl)-2-((1-(N,N-dimethylsulfamoyl)-3-phenyl-1H-pyrazol-5-yl)amino)-4-morpholinofuro[3,2-d]pyrimidine-6-carboxamide (43 mg, 0.065 mmol) in DCM (4 mL) was added TFA (2 mL). The reaction mixture was stirred at room temperature overnight. The completion was monitored by LC-MS. The reaction mixture was quenched with a saturated NaHCO₃ solution and the pH was adjusted to 10. The aqueous solution was extracted with DCM/MeOH (15/1, 3×20 mL). The combined organic phase was dried over anhydrous Na₂SO₄, filtrated and concentrated. After concentration and slurry in MeOH, the impure azetidin-1-yl(4-morpholino-2-((3-phenyl-1H-pyrazol-5-yl)amino)furo[3,2-d]pyrimidin-6-yl)methanone (Compound 52, 21 mg, 0.048 mmol) was obtained as a yellow solid. LC-MS (ESI+): m/z 406 (MH⁺).

### 1.5) Synthesis of Compound 52, 4-morpholino-2-((3-phenyl-1H-pyrazol-5-yl)amino)furo[3,2-d]pyrimidine-6-carboxamide hydrochloride

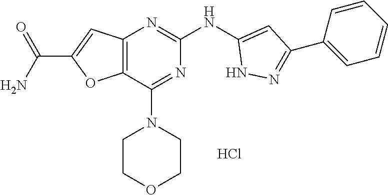 [SMILES: NC(=O)c1cc2nc(Nc3cc(-c4ccccc4)n[nH]3)nc(N3CCOCC3)c2o1 ; Cl]

To a solution of impure azetidin-1-yl(4-morpholino-2-((3-phenyl-1H-pyrazol-5-yl)amino)furo [3,2-d]pyrimidin-6-yl)methanone (21 mg, 0.048 mmol) in DCM (4 mL) was added HCl/Et₂O (2 mL). The reaction mixture was stirred at room temperature for 2 h. After concentration and slurry in MeOH/Et₂O (1/20, 2 mL), 4-morpholino-2-((3-phenyl-1H-pyrazol-5-yl)amino)furo[3,2-d]pyrimidine-6-carboxamide hydrochloride (Compound 52, 17.1 mg, 0.039 mmol) was obtained as a yellow solid. LC-MS (ESI+): m/z 406 (MH⁺). ¹HNMR (300 MHz, CD₃OD) δ 7.74 (d, J=9.9 Hz, 2H), 7.70 (s, 1H), 7.60-7.42 (m, 3H), 6.42 (s, 1H), 4.48-4.14 (m, 4H), 3.91-3.85 (m, 4H).

### Example 15: Compound 53 Using General Synthetic Route 15

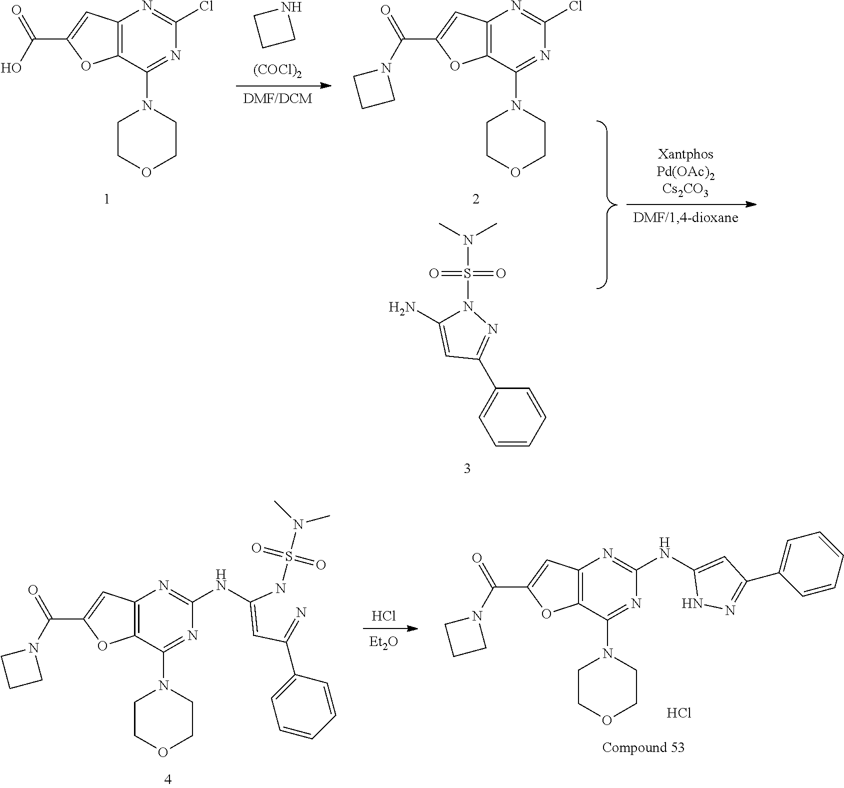

### 1.1) Synthesis of azetidin-1-yl(2-chloro-4-morpholinofuro[3,2-d]pyrimidin-6-yl)methanone

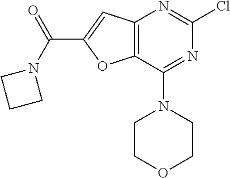 [SMILES: O=C(c1cc2nc(Cl)nc(N3CCOCC3)c2o1)N1CCC1]

A solution of 2-chloro-4-morpholinofuro[3,2-d]pyrimidine-6-carboxylic acid (200 mg, 0.71 mmol) in DCM was added oxalyl dichloride (182 mg, 1.4 mmol) and one drop of DMF. The mixture was stirred at room temperature for 6 h. The solution was concentrated, and the resulting residue was dissolved in DCM (15 mL). To the solution was added azetidine (60 mg, 1.06 mmol) and followed by triethylamine (107 mg, 1.06 mmol). The completion of the reaction was monitored by TLC. The reaction mixture was quenched with water and extracted with DCM (3×20 mL). The combined organic phase was dried over anhydrous Na₂SO₄, filtrated and concentrated under reduced pressure. The residue was purified by silica gel column chromatography with a gradient elution of 1% MeOH/DCM to 5% MeOH/DCM to provide azetidin-1-yl(2-chloro-4-morpholinofuro [3,2-d]pyrimidin-6-yl)methanone (75.8 mg, 0.25 mmol) as a yellow solid. LC-MS (ESI+): m/z 323/325 (MH⁺). ¹HNMR (300 MHz, CDCl₃) δ 7.19 (s, 1H), 4.58-4.52 (m, 2H), 4.35-4.27 (m, 2H), 4.07-4.04 (m, 4H), 3.86-3.83 (m, 4H), 2.52-2.45 (m, 2H).

### 1.2) Synthesis of 5-((6-(azetidine-1-carbonyl)-4-morpholinofuro[3,2-d]pyrimidin-2-yl)amino)-N,N-dimethyl-3-phenyl-1H-pyrazole-1-sulfonamide

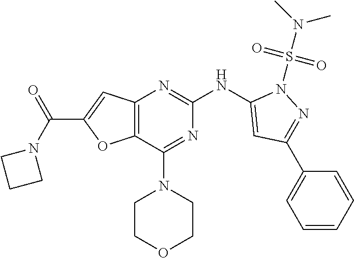 [SMILES: CN(C)S(=O)(=O)n1nc(-c2ccccc2)cc1Nc1nc(N2CCOCC2)c2oc(C(=O)N3CCC3)cc2n1]

A suspension of azetidin-1-yl(2-chloro-4-morpholinofuro[3,2-d]pyrimidin-6-yl)methanone (3×50 mg, 0.47 mmol), 5-amino-N,N-dimethyl-3-phenyl-1H-pyrazole-1-sulfonamide (148 mg, 0.56 mmol), Cs₂CO₃ (344 mg, 1.06 mmol), Pd(OAc)₂ (10 mg, 0.047 mmol) and Xantphos (25 mg, 0.047 mmol) in DMF/1,4-dioxane (7/1, 4 mL) was heated to 50° C. for 50 min under microwave condition. The reaction mixture was concentrated directly and purified by silica gel column chromatography with a gradient elution of 1% MeOH/DCM to 5% MeOH/DCM to provide 5-((6-(azetidine-1-carbonyl)-4-morpholinofuro[3,2-d]pyrimidin-2-yl)amino)-N,N-dimethyl-3-phenyl-1H-pyrazole-1-sulfonamide (143 mg, 0.26 mmol) as a yellow solid. LC-MS (ESI+): m/z 553 (MH⁺). ¹HNMR (300 MHz, CDCl₃) δ 8.70 (s, 1H), 7.91 (d, J=6.9 Hz, 2H), 7.46-7.36 (m, 4H), 7.22 (s, 1H), 4.07-4.04 (m, 4H), 3.87-3.76 (m, 6H), 3.73-3.67 (m, 2H), 3.06 (s, 6H), 2.11-1.96 (m, 2H).

### 1.3) Synthesis of Compound 53, azetidin-1-yl(4-morpholino-2-((3-phenyl-1H-pyrazol-5-yl)amino) furo[3,2-d]pyrimidin-6-yl)methanone hydrochloride

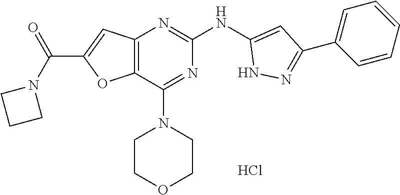 [SMILES: O=C(c1cc2nc(Nc3cc(-c4ccccc4)n[nH]3)nc(N3CCOCC3)c2o1)N1CCC1 ; Cl]

To a solution of 5-((6-(azetidine-1-carbonyl)-4-morpholinofuro[3,2-d]pyrimidin-2-yl)amino)-N,N-dimethyl-3-phenyl-1H-pyrazole-1-sulfonamide (65 mg, 0.11 mmol) in DCM (4 mL) was added HCl/Et₂O (2 mL). The reaction mixture was stirred at room temperature for 2 h. After concentration and slurry in MeOH/Et₂O (1/20, 2 mL), azetidin-1-yl(4-morpholino-2-((3-phenyl-1H-pyrazol-5-yl) amino)furo[3,2-d] pyrimidin-6-yl)methanone hydrochloride (Compound 53, 30.1 mg, 0.06 mmol) was obtained as a yellow solid. LC-MS (ESI+): m/z 446 (MH⁺). ¹HNMR (300 MHz, CD₃OD) δ 7.71 (d, J=7.2 Hz, 2H), 7.64-7.40 (m, 4H), 6.42 (s, 1H), 4.87-4.65 (m, 2H), 4.38-4.13 (m, 6H), 3.91-3.83 (m, 4H), 2.54-2.43 (m, 2H).

### Example 16: Compound 55 Using General Synthetic Route 16

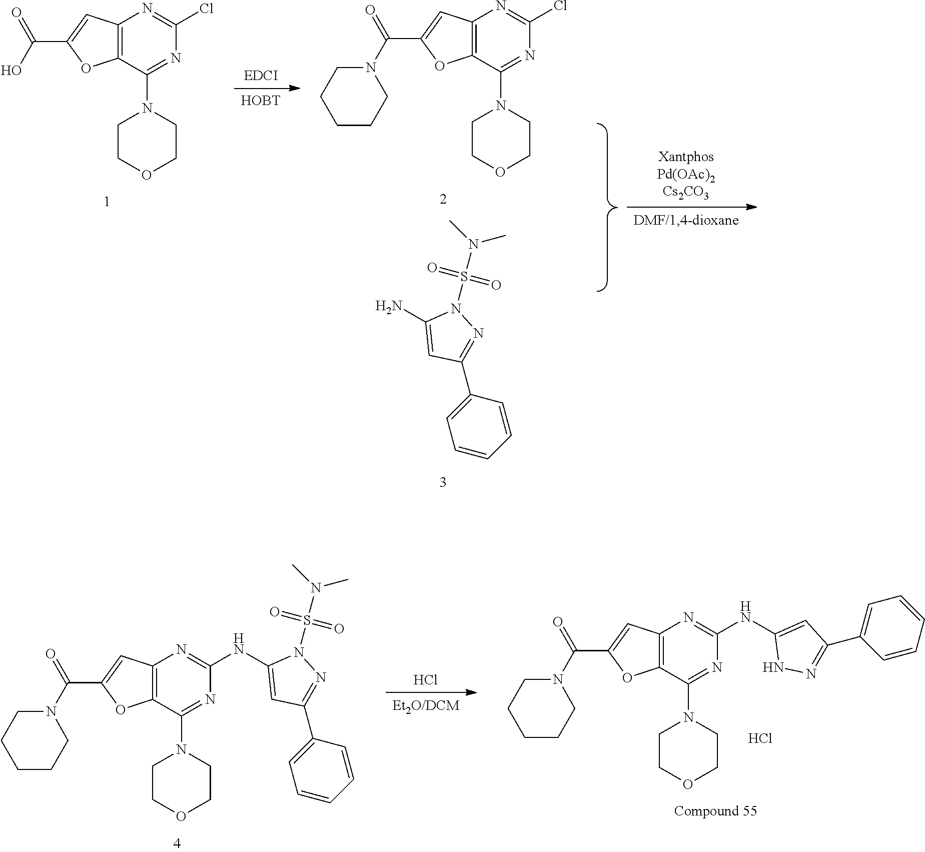

### 1.1) Synthesis of (2-chloro-4-morpholinofuro[3,2-d]pyrimidin-6-yl)(piperidin-1-yl)methanone

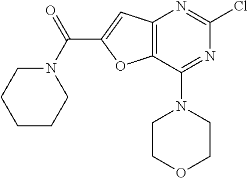 [SMILES: O=C(c1cc2nc(Cl)nc(N3CCOCC3)c2o1)N1CCCCC1]

A solution of 2-chloro-4-morpholinofuro[3,2-d]pyrimidine-6-carboxylic acid (200 mg, 0.71 mmol), piperidine (61 mg, 0.71 mmol), HOBT (245 mg, 1.76 mmol), EDCl (340 mg, 1.76 mmol) in DMF (12 mL) was stirred at room temperature overnight. The completion of the reaction was monitored by TLC. The reaction mixture was diluted with water and extracted with DCM/MeOH (15/1, 3×20 mL). The combined organic phase was dried over anhydrous Na₂SO₄, filtrated and concentrated under reduced pressure. The residue was purified by silica gel column chromatography with a gradient elution of 1% MeOH/DCM to 5% MeOH/DCM to provide (2-chloro-4-morpholinofuro[3,2-d]pyrimidin-6-yl)(piperidin-1-yl)methanone (171 mg, 0.49 mmol) as a white solid. LC-MS (ESI+): m/z 351/353 (MH⁺). ¹HNMR (300 MHz, CDCl₃) δ 6.99 (s, 1H), 4.08-4.03 (m, 4H), 3.85-3.79 (m, 4H), 3.75-3.58 (m, 4H), 1.78-1.66 (m, 6H).

### 1.2) Synthesis of N,N-dimethyl-5-((4-morpholino-6-(piperidine-1-carbonyl)furo[3,2-d]pyrimidin-2-yl)amino)-3-phenyl-1H-pyrazole-1-sulfonamide

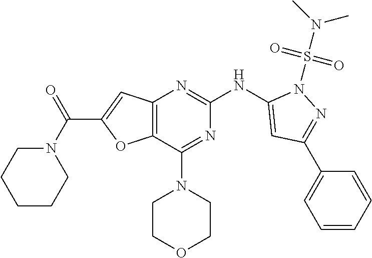 [SMILES: CN(C)S(=O)(=O)n1nc(-c2ccccc2)cc1Nc1nc(N2CCOCC2)c2oc(C(=O)N3CCCCC3)cc2n1]

A suspension of (2-chloro-4-morpholinofuro[3,2-d]pyrimidin-6-yl)(piperidin-1-yl)methanone (100 mg, 0.29 mmol), 5-amino-N,N-dimethyl-3-phenyl-1H-pyrazole-1-sulfonamide (91 mg, 0.34 mmol), Cs₂CO₃ (214 mg, 0.66 mmol), Pd(OAc)₂ (6.4 mg, 0.029 mmol) and Xantphos (16.5 mg, 0.029 mmol) in DMF/1,4-dioxane (7/1, 4 mL) was heated to 90° C. for 45 min under microwave condition. The reaction mixture was concentrated directly and purified by silica gel column chromatography with a gradient elution of 1% MeOH/DCM to 5% MeOH/DCM to provide N,N-dimethyl-5-((4-morpholino-6-(piperidine-1-carbonyl)furo[3,2-d]pyrimidin-2-yl) amino)-3-phenyl-1H-pyrazole-1-sulfonamide (53 mg, 0.09 mmol) as a colorless oil. LC-MS (ESI+): m/z 581 (MH⁺). ¹HNMR (300 MHz, CDCl₃) δ 8.70 (s, 1H), 7.90 (d, J=6.9 Hz, 2H), 7.45-7.34 (m, 4H), 7.02 (s, 1H), 4.04-4.02 (m, 4H), 3.87-3.85 (m, 4H), 3.79-3.70 (m, 4H), 3.06 (s, 6H), 1.76-1.68 (m, 6H).

### 1.3) Synthesis of Compound 55, (4-morpholino-2-((3-phenyl-1H-pyrazol-5-yl)amino)furo[3,2-d]pyrimidin-6-yl)(piperidin-1-yl)methanone hydrochloride

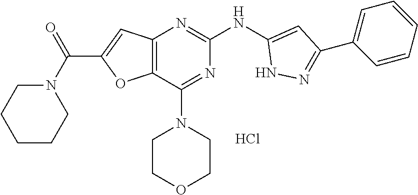 [SMILES: O=C(c1cc2nc(Nc3cc(-c4ccccc4)n[nH]3)nc(N3CCOCC3)c2o1)N1CCCCC1 ; Cl]

To a solution of N,N-dimethyl-5-((4-morpholino-6-(piperidine-1-carbonyl)furo[3,2-d]pyrimidin-2-yl)amino)-3-phenyl-1H-pyrazole-1-sulfonamide (53 mg, 0.09 mmol) in DCM (4 mL) was added HCl/Et₂O (2 mL). The reaction mixture was stirred at room temperature for 2 h. After concentration and slurry in MeOH/Et₂O (1/20, 2 mL), (4-morpholino-2-((3-phenyl-1H-pyrazol-5-yl)amino)furo [3,2-d]pyrimidin-6-yl)(piperidin-1-yl)methanone hydrochloride (Compound 55, 17.6 mg, 0.35 mmol) was obtained as a white solid. LC-MS (ESI+): m/z 474 (MH⁺). ¹HNMR (300 MHz, DMSO-d₆) δ 7.78 (d, J=7.5 Hz, 2H), 7.59-7.45 (m, 2H), 7.40-7.36 (m, 1H), 7.29 (s, 1H), 6.61 (s, 1H), 4.19-4.03 (m, 4H), 3.88-3.79 (m, 4H), 3.59-3.47 (m, 4H), 1.76-1.47 (m, 6H).

### Example 17: Compound 64 Using General Synthetic Route 17

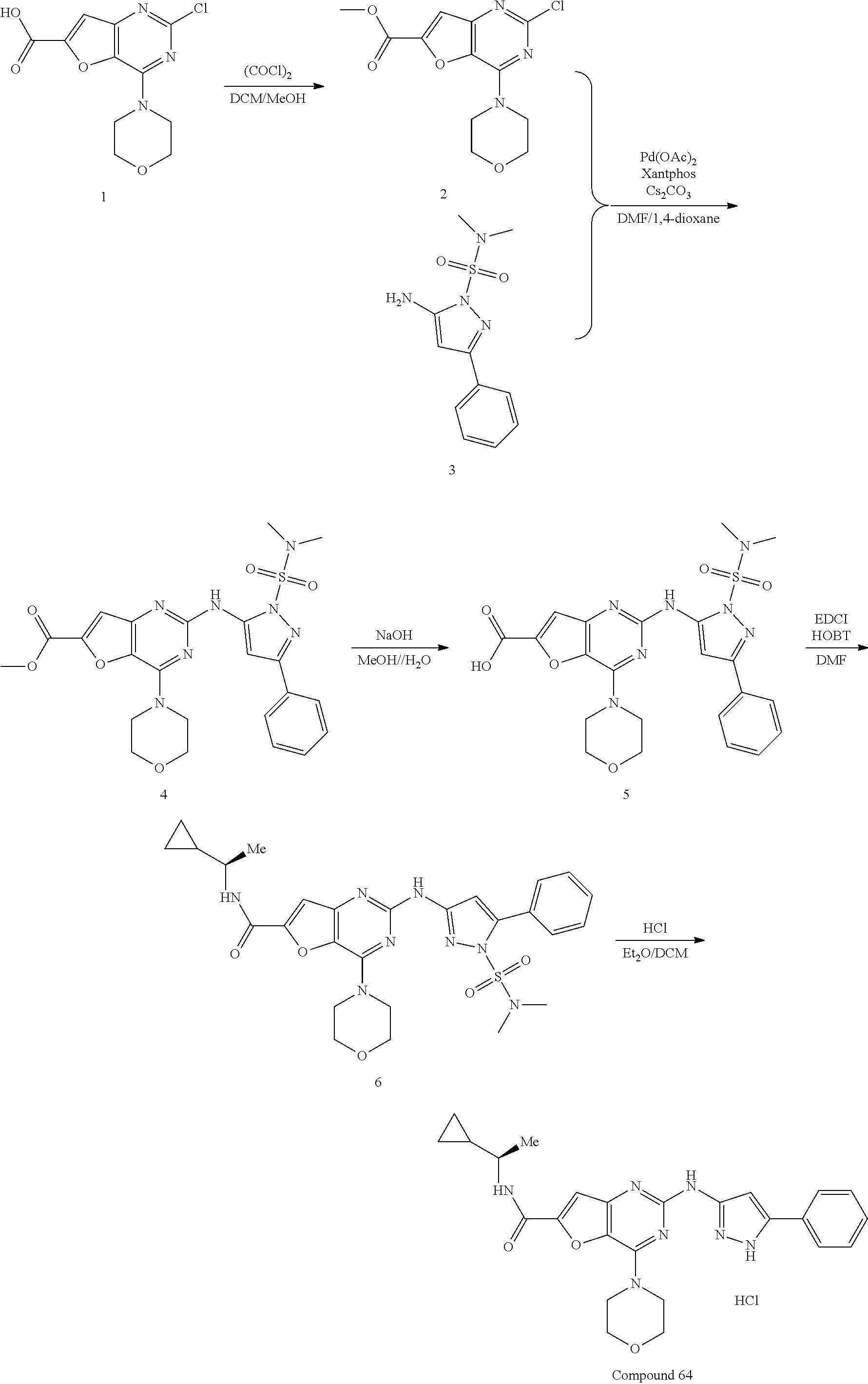

### 1.1) Synthesis of methyl 2-chloro-4-morpholinofuro[3,2-d]pyrimidine-6-carboxylate

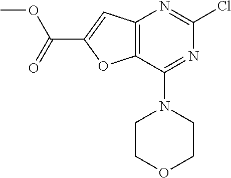 [SMILES: COC(=O)c1cc2nc(Cl)nc(N3CCOCC3)c2o1]

A solution of 2-chloro-4-morpholinofuro[3,2-d]pyrimidine-6-carboxylic acid (1.0 g, 3.5 mmol) in DCM was added oxalyl dichloride (912.0 mg, 7.0 mmol) and one drop of DMF. The mixture was stirred at room temperature for 6 h. The solution was concentrated directly and the residue was dissolved in DCM (15 mL). To the solution was added methanol (80 mL) and followed by triethylamine (1.07 g, 10.5 mmol). The completion of the reaction was monitored by TLC. The reaction was quenched with water (10 ml) and a large amount of yellow solid was precipitated. After filtration, 800 mg of methyl 2-chloro-4-morpholinofuro[3,2-d]pyrimidine-6-carboxylate was obtained. LC-MS (ESI+): m/z 298/300 (MH⁺). ¹HNMR (300 MHz, CDCl₃) δ 7.40 (s, 1H), 4.09-4.01 (m, 4H), 3.99 (s, 3H), 3.87-3.84 (m, 4H).

### 1.2) Synthesis of methyl 2-((1-(N,N-dimethylsulfamoyl)-3-phenyl-1H-pyrazol-5-yl)amino)-4-morpholinofuro[3,2-d]pyrimidine-6-carboxylate

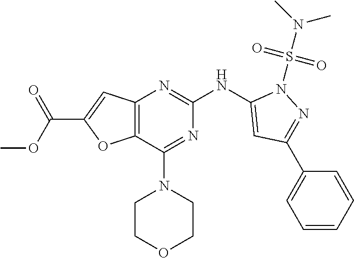 [SMILES: COC(=O)c1cc2nc(Nc3cc(-c4ccccc4)nn3S(=O)(=O)N(C)C)nc(N3CCOCC3)c2o1]

A suspension of methyl 2-chloro-4-morpholinofuro[3,2-d]pyrimidine-6-carboxylate (400 mg, 1.35 mmol), 5-amino-N,N-dimethyl-3-phenyl-1H-pyrazole-1-sulfonamide (430 mg, 1.620 mmol), Cs₂CO₃ (1.1 g, 3.38 mmol), Pd(OAc)₂ (30 mg, 0.135 mmol) and Xantphos (80 mg, 0.135 mmol) in DMF/1,4-dioxane (1/7, 40 mL) was heated to 80° C. for 2 h. The reaction mixture was diluted with water and extracted with DCM (3×20 mL). The combined organic phase was dried over anhydrous Na₂SO₄, filtrated and concentrated under reduced pressure. The residue was purified by silica gel column chromatography with a gradient elution of 50% EtOAc/PE to DCM to provide 2-((1-(N,N-dimethylsulfamoyl)-3-phenyl-1H-pyrazol-5-yl)amino)-4-morpholinofuro[3,2-d]pyrimidine-6-carboxylate (250 mg, 0.47 mmol) as a yellow solid. LC-MS (ESI+): m/z 528 (MH⁺).

### 1.3) Synthesis of 2-((1-(N,N-dimethylsulfamoyl)-3-phenyl-1H-pyrazol-5-yl)amino)-4-morpholinofuro[3,2-d]pyrimidine-6-carboxylic acid

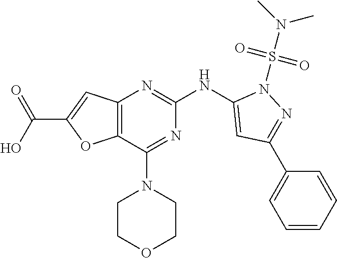 [SMILES: CN(C)S(=O)(=O)n1nc(-c2ccccc2)cc1Nc1nc(N2CCOCC2)c2oc(C(=O)O)cc2n1]

To a solution of 2-((1-(N,N-dimethylsulfamoyl)-3-phenyl-1H-pyrazol-5-yl)amino)-4-morpholinofuro[3,2-d]pyrimidine-6-carboxylate (250 mg, 0.47 mmol) in MeOH/DCM (1/2, 30 mL) was added NaOH aqueous solution (2M, 0.3 mL). The completion of the reaction was monitored by TLC. The reaction mixture was quenched with HCl aqueous solution and adjusted the pH to 5. The aqueous phase was extracted with DCM (3×20 mL). The combined organic phase was dried over anhydrous Na₂SO₄, filtrated and concentrated under reduced pressure. The residue was purified by slurry using Et₂O to provide 2-((1-(N,N-dimethylsulfamoyl)-3-phenyl-1H-pyrazol-5-yl)amino)-4-morpholinofuro[3,2-d]pyrimidine-6-carboxylic acid (200 mg, 0.39 mmol) as a yellow solid. LC-MS (ESI+): m/z 514 (MH⁺).

### 1.4) Synthesis of (R)—N-(1-cyclopropylethyl)-2-((1-(N,N-dimethylsulfamoyl)-3-phenyl-1H-pyrazol-5-yl)amino)-4-morpholinofuro[3,2-d]pyrimidine-6-carboxamide

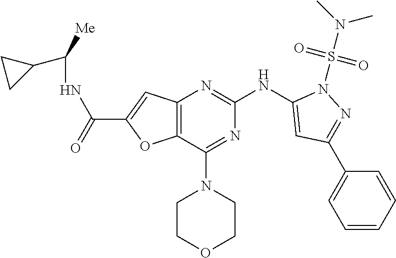 [SMILES: C[C@@H](NC(=O)c1cc2nc(Nc3cc(-c4ccccc4)nn3S(=O)(=O)N(C)C)nc(N3CCOCC3)c2o1)C1CC1]

A solution of 2-((1-(N,N-dimethylsulfamoyl)-3-phenyl-1H-pyrazol-5-yl)amino)-4-morpholinofuro [3,2-d]pyrimidine-6-carboxylic acid (150 mg, 0.29 mmol), (R)-1-cyclopropylethanamine (30 mg, 0.35 mmol), HOBT (99 mg, 0.73 mmol) and EDCl (140 mg, 0.73 mmol) in DMF (3 mL) was stirred at room temperature overnight. The completion of the reaction was monitored by TLC. The reaction was quenched with water (10 ml) and extracted with DCM/MeOH (15/1, 3×20 mL). The combined organic phase was dried over anhydrous Na₂SO₄, filtrated and concentrated under reduced pressure. The residue was purified by silica gel column chromatography with a gradient elution of 1% MeOH/DCM to 5% MeOH/DCM to provide (R)—N-(1-cyclopropylethyl)-2-((1-(N,N-dimethylsulfamoyl)-5-phenyl-1H-pyrazol-3-yl)amino)-4-morpholinofuro[3,2-d]pyrimidine-6-carboxamide (80 mg, 0.14 mmol). LC-MS (ESI+): m/z 581 (MH⁺). ¹HNMR (300 MHz, CDCl₃) (8.72 (s, 1H), 7.90 (d, J=6.6 Hz, 2H), 7.47-7.39 (m, 3H), 7.35 (s, 1H), 6.24 (d, J=8.1 Hz, 1H), 4.10-4.04 (m, 4H), 3.89-3.84 (m, 4H), 3.59-3.56 (m, 1H), 3.07 (s, 6H), 1.36 (d, J=6.6 Hz, 3H), 0.97-0.95 (m, 1H), 0.60-0.52 (m, 2H), 0.47-0.32 (m, 2H).

### 1.5) Synthesis of Compound 64, (R)—N-(1-cyclopropylethyl)-4-morpholino-2-((5-phenyl-1H-pyrazol-3-yl)amino)furo[3,2-d]pyrimidine-6-carboxamide hydrochloride

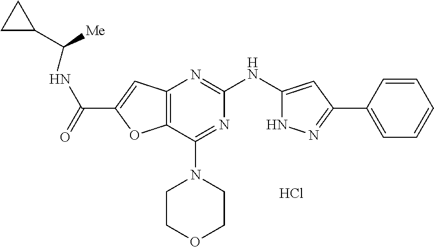 [SMILES: C[C@@H](NC(=O)c1cc2nc(Nc3cc(-c4ccccc4)n[nH]3)nc(N3CCOCC3)c2o1)C1CC1 ; Cl]

To a solution of (R)—N-(1-cyclopropylethyl)-2-((1-(N,N-dimethylsulfamoyl)-5-phenyl-1H-pyrazol-3-yl)amino)-4-morpholinofuro[3,2-d]pyrimidine-6-carboxamide (80 mg, 0.14 mmol) in DCM (4 mL) was added HCl/Et₂O (2 mL). The reaction mixture was stirred at room temperature for 2 h. After concentration and slurry in MeOH/Et₂O (1/20, 2 mL), (R)—N-(1-cyclopropylethyl)-4-morpholino-2-((5-phenyl-1H-pyrazol-3-yl)amino)furo[3,2-d]pyrimidine-6-carboxamide hydrochloride (Compound 64, 30 mg, 0.06 mmol) was obtained as a white solid. LC-MS (ESI+): m/z 474 (MH⁺). ¹HNMR (300 MHz, DMSO-d₆) δ 10.79 (s, 1H), 8.93 (d, J=8.1 Hz, 1H), 7.80 (d, J=7.5 Hz, 2H), 7.59 (s, 1H), 7.54-7.42 (m, 2H), 7.39-7.37 (m, 1H), 6.61 (s, 1H), 4.16-4.05 (m, 4H), 3.86-3.79 (m, 4H), 3.40-3.30 (m, 1H), 1.28 (d, J=6.9 Hz, 3H), 1.11-1.04 (m, 1H), 0.49-0.40 (m, 2H), 0.39-0.25 (m, 2H).

### Example 18: Compound 65 Using General Synthetic Route 18

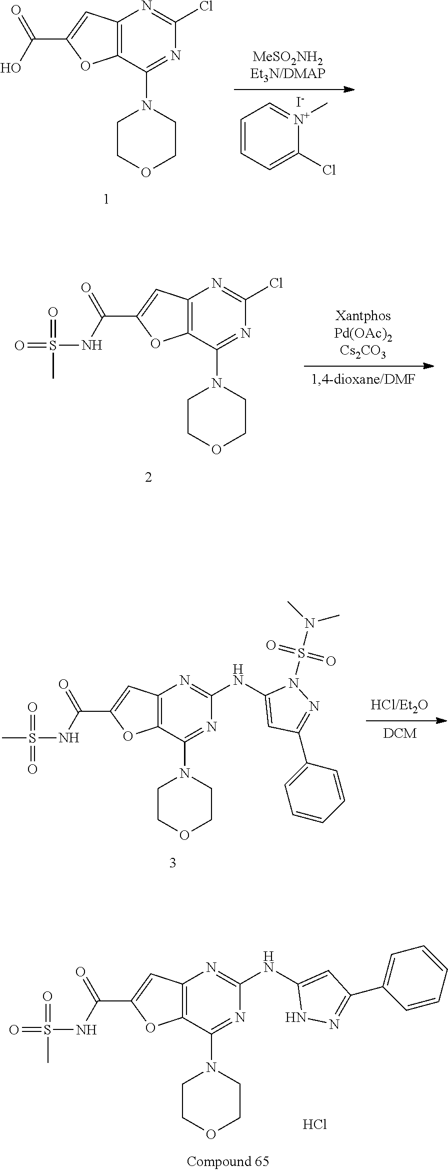

### 1.1) Synthesis of 2-chloro-N-(methylsulfonyl)-4-morpholinofuro[3,2-d]pyrimidine-6-carboxamide

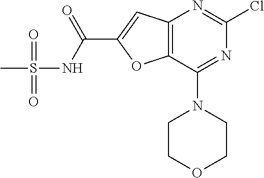 [SMILES: CS(=O)(=O)NC(=O)c1cc2nc(Cl)nc(N3CCOCC3)c2o1]

A solution of 2-chloro-4-morpholinofuro[3,2-d]pyrimidine-6-carboxylic acid (200 mg, 0.71 mmol), methanesulfonamide (134 mg, 1.42 mmol), 2-chloro-1-methylpyridin-1-ium iodide (216 mg, 0.85 mmol), Et₃N (214 mg, 2.12 mmol) and DMAP (4.3 mg, 0.035 mmol) in DCM (25 mL) was stirred at room temperature overnight. The completion of the reaction was monitored by TLC. The reaction was quenched with water (10 ml) and adjusted the pH to 4 using 1 N HCl aqueous solution. The aqueous phase was extracted with DCM/MeOH (15/1, 3×20 mL). The combined organic phase was dried over anhydrous Na₂SO₄, filtrated and concentrated under reduced pressure. The residue was purified by silica gel column chromatography with a gradient elution of 1% MeOH/DCM to 5% MeOH/DCM to provide 2-chloro-N-(methylsulfonyl)-4-morpholinofuro [3,2-d]pyrimidine-6-carboxamide (244 mg, 0.68 mmol) as a yellow solid. LC-MS (ESI+): m/z 361/363 (MH⁺).

### 1.2) Synthesis of 2-((1-(N,N-dimethylsulfamoyl)-3-phenyl-1H-pyrazol-5-yl)amino)-N-(methylsulfonyl)-4-morpholinofuro[3,2-d]pyrimidine-6-carboxamide

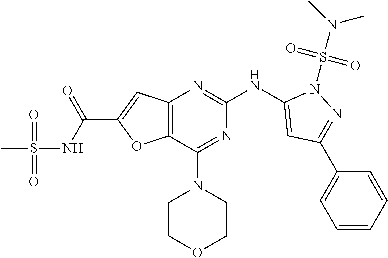 [SMILES: CN(C)S(=O)(=O)n1nc(-c2ccccc2)cc1Nc1nc(N2CCOCC2)c2oc(C(=O)NS(C)(=O)=O)cc2n1]

A suspension of 2-chloro-N-(methylsulfonyl)-4-morpholinofuro[3,2-d]pyrimidine-6-carboxamide (60 mg×3, 0.17 mmol), 5-amino-N,N-dimethyl-3-phenyl-1H-pyrazole-1-sulfonamide (53 mg, 0.2 mmol), Cs₂CO₃ (127 mg, 0.39 mmol), Pd(OAc)₂ (3.8 mg, 0.017 mmol) and Xantphos (9.6 mg, 0.017 mmol) in DMF/1,4-dioxane (7/1, 8 mL) was heated to 100° C. for 40 min under microwave condition. The reaction mixture was concentrated directly and purified by silica gel column chromatography with a gradient elution of 2% MeOH/DCM to 10% MeOH/DCM to provide 2-((1-(N,N-dimethylsulfamoyl)-3-phenyl-1H-pyrazol-5-yl)amino)-N-(methylsulfonyl)-4-morpholinofuro[3,2-d]pyrimidine-6-carboxamide (30 mg, 0.051 mmol) as a white solid. LC-MS (ESI+): m/z 591 (MH⁺). ¹HNMR (300 MHz, CD₃OD) δ 7.86 (d, J=6.9 Hz, 2H), 7.48-7.40 (m, 3H), 7.28 (s, 1H), 7.21 (s, 1H), 4.13-4.07 (m, 4H), 3.89-3.84 (m, 4H), 3.15 (s, 3H), 3.03 (s, 6H).

### 1.3) Synthesis of Compound 65, N-(methylsulfonyl)-4-morpholino-2-((3-phenyl-1H-pyrazol-5-yl) amino)furo[3,2-d]pyrimidine-6-carboxamide hydrochloride

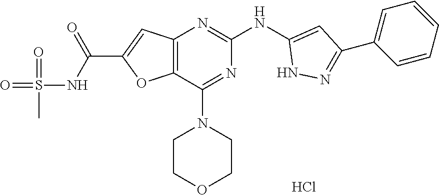 [SMILES: CS(=O)(=O)NC(=O)c1cc2nc(Nc3cc(-c4ccccc4)n[nH]3)nc(N3CCOCC3)c2o1 ; Cl]

To a solution of 2-((1-(N,N-dimethylsulfamoyl)-3-phenyl-1H-pyrazol-5-yl)amino)-N-(methylsulfonyl)-4-morpholinofuro[3,2-d]pyrimidine-6-carboxamide (30 mg, 0.05 mmol) in DCM (4 mL) was added HCl/Et₂O (2 mL). The reaction mixture was stirred at room temperature for 2 h. After concentration and slurry in MeOH/Et₂O (1/20, 2 mL), N-(methylsulfonyl)-4-morpholino-2-((3-phenyl-1H-pyrazol-5-yl)amino)furo[3,2-d]pyrimidine-6-carboxamide hydrochloride (Compound 65, 23.3 mg, 0.082 mmol) was obtained as a white solid. LC-MS (ESI+): m/z 484 (MH⁺). ¹HNMR (300 MHz, CD₃OD) δ 7.75 (s, 1H), 7.72 (d, J=6.9 Hz, 2H), 7.52-7.40 (m, 3H), 6.43 (s, 1H), 4.36-4.18 (m, 4H), 3.91-3.86 (m, 4H), 3.39 (s, 3H).

### Example 19: Compound 66 Using General Synthetic Route 19

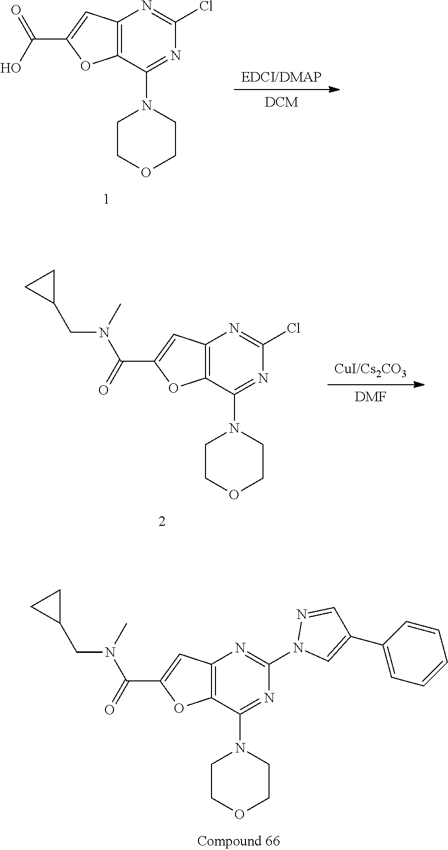

### 1.1) Synthesis of 2-chloro-N-(cyclopropylmethyl)-N-methyl-4-morpholinofuro[3,2-d]pyrimidine-6-carboxamide

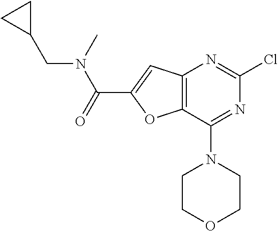 [SMILES: CN(CC1CC1)C(=O)c1cc2nc(Cl)nc(N3CCOCC3)c2o1]

A solution of 2-chloro-4-morpholinofuro[3,2-d]pyrimidine-6-carboxylic acid (270 mg, 0.95 mmol), 1-cyclopropyl-N-methylmethanamine (80 mg, 0.95 mmol), DMAP (292 mg, 2.4 mmol) and EDCl (460 mg, 2.4 mmol) in DCM (20 mL) was stirred at room temperature overnight. The completion of the reaction was monitored by TLC. The reaction mixture was concentrated directly and purified by silica gel column chromatography with a gradient elution of 1% MeOH/DCM to 2% MeOH/DCM to provide 2-chloro-N-(cyclopropylmethyl)-N-methyl-4-morpholinofuro[3,2-d] pyrimidine-6-carboxamide (2 72 mg, 0.78 mmol) as a white solid. LC-MS (ESI+): m/z 351/353 (MH⁺). ¹HNMR (300 MHz, CDCl₃) δ 7.09 (s, 1H), 4.09-4.04 (m, 4H), 3.88-3.82 (m, 4H), 3.48-3.37 (m, 2H), 3.29 (s, 1.5H), 3.20 (s, 1.5H), 1.11-0.98 (m, 1H), 0.65-0.60 (m, 2H), 0.34-0.21 (m, 2H).

### 1.2) Synthesis of Compound 66, N-(cyclopropylmethyl)-N-methyl-4-morpholino-2-(4-phenyl-1H-pyrazol-1-yl)furo[3,2-d]pyrimidine-6-carboxamide

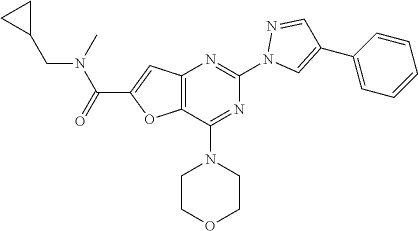 [SMILES: CN(CC1CC1)C(=O)c1cc2nc(-n3cc(-c4ccccc4)cn3)nc(N3CCOCC3)c2o1]

A suspension of 2-chloro-N-(cyclopropylmethyl)-N-methyl-4-morpholinofuro[3,2-d]pyrimidine-6-carboxamide (100 mg, 0.29 mmol), 4-phenyl-1H-pyrazole (41 mg, 0.29 mmol), CuI (11 mg, 0.06 mmol) and Cs₂CO₃ (186 mg, 0.57 mmol) in DMF (10 mL) was stirred at 100° C. overnight. The completion of the reaction was monitored by TLC. The reaction mixture was diluted with water (20 mL) and extracted with DCM/MeOH (15/1, 3×30 mL). The combined organic phase was dried over anhydrous Na₂SO₄, filtrated and concentrated under reduced pressure. The residue was purified by silica gel column chromatography with a gradient elution of 1% MeOH/DCM to 2% MeOH/DCM to provide N-(cyclopropylmethyl)-N-methyl-4-morpholino-2-(4-phenyl-1H-pyrazol-1-yl)furo[3,2-d]pyrimidine-6-carboxamide (Compound 66, 27 mg, 0.059 mmol) as a white solid. LC-MS (ESI+): m/z 459 (MH)). ¹HNMR (300 MHz, DMSO-d₆) δ 9.05 (s, 1H), 8.35 (s, 1H), 7.80 (d, J=7.2 Hz, 2H), 7.44-7.39 (m, 3H), 7.30-7.28 (m, 1H), 4.12-4.06 (m, 4H), 3.85-3.78 (m, 4H), 3.39-3.30 (m, 2H), 3.30 (s, 1.5H), 3.10 (s, 1.5H), 1.10-1.01 (m, 1H), 0.53-0.51 (m, 2H), 0.34-0.15 (m, 2H).

### Example 20: Compound 69 Using General Synthetic Route 20

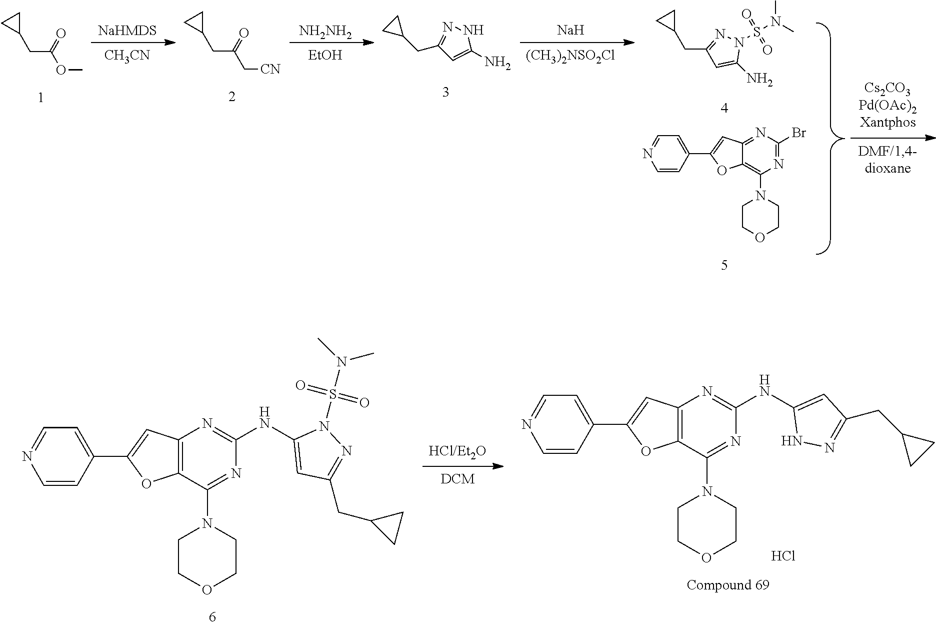

### 1.1) Synthesis of 4-cyclopropyl-3-oxobutanenitrile

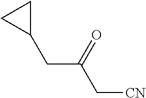

To a solution of acetonitrile (1.08 g, 26.3 mmol) and methyl 2-cyclopropylacetate (2.0 g, 17.5 mmol) in anhydrous THF (40 mL) at 0° C. under N₂ was added NaHDMS (13.2 mL, 26.3 mmol) dropwise. After addition, the solution was stirred at room temperature for 2 h. The completion of the reaction was monitored by TLC. The reaction was quenched with NH₄Cl aqueous solution (20 mL) and extracted with EtOAc (3×30 mL). The combined organic phase was dried over anhydrous Na₂SO₄, filtrated and concentrated under reduced pressure. The resulting residue was purified by silica gel column chromatography with a gradient elution of 10% EtOAc/PE to 33% EtOAc/PE to provide 4-cyclopropyl-3-oxobutanenitrile (1.6 g, 13.1 mmol) as yellow oil. ¹HNMR (300 MHz, CDCl₃) δ 3.34 (s, 2H), 2.29 (d, J=6.9 Hz, 2H), 1.89-1.84 (m, 1H), 0.85-0.79 (m, 2H), 0.06-0.01 (m, 2H).

### 1.2) Synthesis of 3-(cyclopropylmethyl)-1H-pyrazol-5-amine

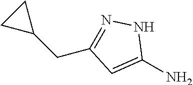

To a solution of 4-cyclopropyl-3-oxobutanenitrile (1.15 g, 7.2 mmol) in EtOH (40 mL) was added NH₂NH₂.H₂O (0.98 g, 19.5 mmol). The reaction mixture was heated to reflux overnight. The completion of the reaction was monitored by TLC. The reaction mixture was concentrated directly and purified by silica gel column chromatography with a gradient elution of 1% MeOH/DCM to 3% MeOH/DCM to provide 3-(cyclopropylmethyl)-1H-pyrazol-5-amine (1.64 g, 0.012 mmol) as a yellow oil. LC-MS (ESI+): m/z 138 (MH⁺). ¹HNMR (300 MHz, CDCl₃) δ 5.51 (m, 1H), 4.41 (brs, 2H), 2.47 (d, J=6.9 Hz, 2H), 1.02-0.90 (m, 1H), 0.59-0.56 (m, 2H), 0.22-0.17 (m, 2H).

### 1.3) Synthesis of 5-amino-3-(cyclopropylmethyl)-N,N-dimethyl-1H-pyrazole-1-sulfonamide

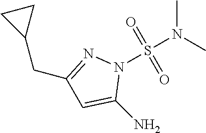 [SMILES: CN(C)S(=O)(=O)n1nc(CC2CC2)cc1N]

To a solution of 3-(cyclopropylmethyl)-1H-pyrazol-5-amine (1.0 g, 7.25 mmol) in THF (30 mL) at 0° C. was added NaH (521 mg, 8.7 mmol). After stirred at 0° C. for 1 h, to the solution was added dimethylsulfamoyl chloride (1.14 g, 7.97 mmol). The completion of the reaction was monitored by TLC. The reaction mixture was quenched with water and extracted with EtOAc (3×50 mL). The combined organic phase was dried over anhydrous Na₂SO₄, filtrated and concentrated under reduced pressure. The resulting residue was purified by silica gel column chromatography with a gradient elution of 10% EtOAc/PE to 33% EtOAc/PE to provide 5-amino-3-(cyclopropylmethyl)-N,N-dimethyl-1H-pyrazole-1-sulfonamide (540 mg, 2.2 mmol). LC-MS: m/z 245 (MH⁺). ¹HNMR (300 MHz, CDCl₃) δ 5.79 (s, 1H), 3.83 (brs, 2H), 2.94 (s, 6H), 2.71 (d, J=6.9 Hz, 2H), 1.08-1.01 (m, 1H), 0.60-0.54 (m, 2H), 0.23-0.19 (m, 2H).

### 1.4) Synthesis of 3-(cyclopropylmethyl)-N,N-dimethyl-5-((4-morpholino-6-(pyridin-4-yl) furo[3,2-d]pyrimidin-2-yl)amino)-1H-pyrazole-1-sulfonamide

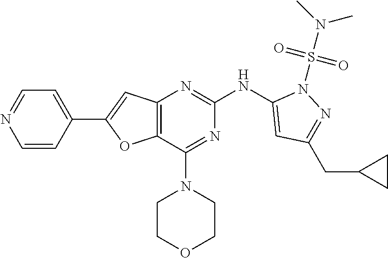 [SMILES: CN(C)S(=O)(=O)n1nc(CC2CC2)cc1Nc1nc(N2CCOCC2)c2oc(-c3ccncc3)cc2n1]

A suspension of 2-bromo-4-morpholino-6-(pyridin-4-yl)furo[3,2-d]pyrimidine (200 mg, 0.56 mmol), 5-amino-3-(cyclopropylmethyl)-N,N-dimethyl-1H-pyrazole-1-sulfonamide (162 mg, 0.67 mmol), Cs₂CO₃ (365 mg, 1.12 mmol), Pd(OAc)₂ (12 mg, 0.056 mmol) and Xantphos (32 mg, 0.056 mmol) in DMF/1,4-dioxane (7/1, 8 mL) was heated to 85° C. for 40 min under microwave condition. The reaction mixture was concentrated directly and purified by silica gel column chromatography with a gradient elution of 1% MeOH/DCM to 5% MeOH/DCM to provide 3-(cyclopropylmethyl)-N,N-dimethyl-5-((4-morpholino-6-(pyridin-4-yl)furo[3,2-d] pyrimidin-2-yl)amino)-1H-pyrazole-1-sulfonamide (47 mg, 0.09 mmol) as a white solid. LC-MS (ESI+): m/z 525 (MH⁺). ¹HNMR (300 MHz, CDCl₃) δ 8.74 (d, J=5.7 Hz, 1H), 8.69 (s, 1H), 7.64 (d, J=6.0 Hz, 1H), 7.18 (s, 1H), 6.85 (s, 1H), 4.12-4.07 (m, 4H), 3.92-3.88 (m, 4H), 2.99 (s, 6H), 2.54 (d, J=7.2 Hz, 2H), 1.11-1.02 (m, 1H), 0.57-0.53 (m, 2H), 0.28-0.23 (m, 2H).

### 1.5) Synthesis of Compound 69, N-(3-(cyclopropylmethyl)-1H-pyrazol-5-yl)-4-morpholino-6-(pyridin-4-yl)furo[3,2-d]pyrimidin-2-amine hydrochloride

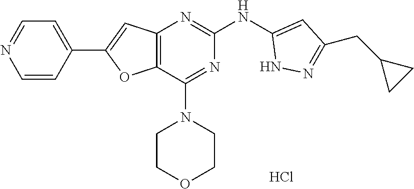 [SMILES: c1cc(-c2cc3nc(Nc4cc(CC5CC5)n[nH]4)nc(N4CCOCC4)c3o2)ccn1 ; Cl]

To a solution of 3-(cyclopropylmethyl)-N,N-dimethyl-5-((4-morpholino-6-(pyridin-4-yl) furo[3,2-d]pyrimidin-2-yl)amino)-1H-pyrazole-1-sulfonamide (47 mg, 0.09 mmol) in DCM (4 mL) was added HCl/Et₂O (2 mL). The reaction mixture was stirred at room temperature for 2 h. After concentration and slurry in MeOH/Et₂O (1/20, 2 mL), N-(3-(cyclopropylmethyl)-1H-pyrazol-5-yl)-4-morpholino-6-(pyridin-4-yl)furo[3,2-d] pyrimidin-2-amine hydrochloride (Compound 69, 32.5 mg, 0.072 mmol) was obtained as a yellow solid. LC-MS (ESI+): m/z 418 (MH⁺). ¹HNMR (300 MHz, CD₃OD) δ 8.98 (d, J=6.9 Hz, 2H), 8.58 (d, J=6.9 Hz, 2H), 8.06 (s, 1H), 6.00 (s, 1H), 4.37-4.27 (m, 4H), 3.94-3.90 (m, 4H), 2.61 (d, J=6.9 Hz, 2H), 1.09-1.02 (m, 1H), 0.62-0.54 (m, 2H), 0.29-0.24 (m, 2H).

### Example 21: Compound 78 Using General Synthetic Route 21

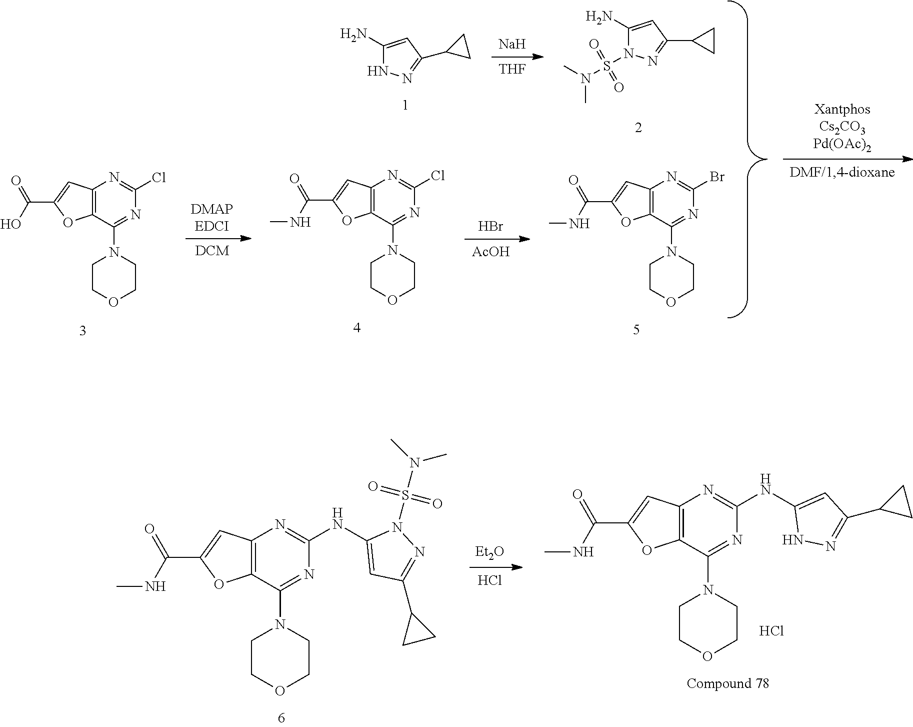

### 1.1) Synthesis of 5-amino-3-cyclopropyl-N,N-dimethyl-1H-pyrazole-1-sulfonamide

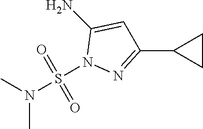

To a solution of 3-cyclopropyl-1H-pyrazol-5-amine (500 mg, 4.06 mmol) in THE (30 mL) at 0° C. was added NaH (243 mg, 6.1 mmol). After stirred at 0° C. for 1 h, to the solution was added dimethylsulfamoyl chloride (755 mg, 5.28 mmol). The completion of the reaction was monitored by TLC. The reaction mixture was quenched with water and extracted with EtOAc (3×50 mL). The combined organic phase was dried over anhydrous Na₂SO₄, filtrated and concentrated under reduced pressure. The resulting residue was purified by silica gel column chromatography with a gradient elution of 10% EtOAc/PE to 25% EtOAc/PE to provide 5-amino-3-cyclopropyl-N,N-dimethyl-1H-pyrazole-1-sulfonamide (372 mg, 1.6 mmol). LC-MS: m/z 231 (MH⁺). ¹HNMR (300 MHz, CDCl₃) δ 5.05 (s, 1H), 4.71 (brs, 2H), 2.96 (s, 6H), 1.84-1.80 (m, 1H), 0.93-0.86 (m, 2H), 0.72-0.67 (m, 2H).

### 1.2) Synthesis of 2-chloro-N-methyl-4-morpholinofuro[3,2-d]pyrimidine-6-carboxamide

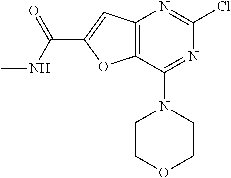 [SMILES: CNC(=O)c1cc2nc(Cl)nc(N3CCOCC3)c2o1]

A solution of 2-chloro-4-morpholinofuro[3,2-d]pyrimidine-6-carboxylic acid (400 mg, 1.4 mmol), methanamine hydrochloride (113 mg, 1.70 mmol), EDCl (678 mg, 3.5 mmol) and DMAP (431 mg, 3.5 mmol) in DCM (3 mL) was stirred at room temperature overnight. The completion of the reaction was monitored by TLC. The reaction mixture was quenched with water (10 ml) and extracted with DCM/MeOH (15/1, 3×20 mL). The combined organic phase was dried over anhydrous Na₂SO₄, filtrated and concentrated under reduced pressure. The residue was purified by silica gel column chromatography with a gradient elution of 1% MeOH/DCM to 5% MeOH/DCM to provide 2-chloro-N-methyl-4-morpholinofuro[3,2-d]pyrimidine-6-carboxamide (230 mg, 0.78 mmol). LC-MS (ESI+): m/z 297/299 (MH⁺). ¹HNMR (300 MHz, CDCl₃) δ 7.35 (s, 1H), 6.34 (brs, 1H), 4.08-4.03 (m, 4H), 3.88-3.83 (m, 4H), 3.07 (d, J=5.1 Hz, 3H).

### 1.3) Synthesis of 2-bromo-N-methyl-4-morpholinofuro[3,2-d]pyrimidine-6-carboxamide

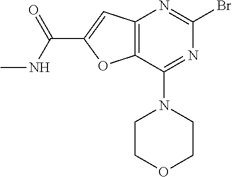 [SMILES: CNC(=O)c1cc2nc(Br)nc(N3CCOCC3)c2o1]

A solution of 2-chloro-N-methyl-4-morpholinofuro[3,2-d]pyrimidine-6-carboxamide (230 mg, 0.78 mmol) in HBr/AcOH (33 wt. % in Acetic acid, 3 mL) was heated to reflux for 3.5 h. The completion of the reaction was monitored by LC-MS. The reaction mixture was quenched with a saturated NaHCO₃ aqueous solution and the pH was adjusted to 8. The aqueous solution was extracted with DCM/MeOH (15/1, 3×20 mL). The combined organic phase was dried over anhydrous Na₂SO₄, filtrated and concentrated under reduced pressure to provide 245 mg of crude 2-bromo-N-methyl-4-morpholinofuro[3,2-d]pyrimidine-6-carboxamide as a brown solid. The crude product was used directly for the next step without further purification. LC-MS (ESI+): m/z 341/343 (MH⁺).

### 1.4) Synthesis of 2-((3-cyclopropyl-1-(N,N-dimethylsulfamoyl)-1H-pyrazol-5-yl)amino)-N-methyl-4-morpholinofuro[3,2-d]pyrimidine-6-carboxamide

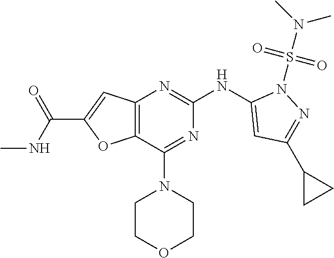 [SMILES: CNC(=O)c1cc2nc(Nc3cc(C4CC4)nn3S(=O)(=O)N(C)C)nc(N3CCOCC3)c2o1]

A solution of 2-bromo-N-methyl-4-morpholinofuro[3,2-d]pyrimidine-6-carboxamide (50 mg×3, 0.15 mmol), 5-amino-3-cyclopropyl-N,N-dimethyl-1H-pyrazole-1-sulfonamide (40 mg, 0.18 mmol), Cs₂CO₃ (110 mg, 0.35 mmol), Pd(OAc)₂ (3.5 mg, 0.015 mmol) and Xantphos (8.5 mg, 0.015 mmol) in DMF/1,4-dioxane (7/1, 4 mL) was heated to 80° C. for 20 min under microwave condition. The reaction mixture was concentrated directly and purified by silica gel column chromatography with a gradient elution of 1% MeOH/DCM to 5% MeOH/DCM to provide 2-((3-cyclopropyl-1-(N,N-dimethylsulfamoyl)-1H-pyrazol-5-yl)amino)-N-methyl-4-morpholinofuro[3,2-d]pyrimidine-6-carboxamide (62 mg, 0.13 mmol) as a white solid. LC-MS (ESI+): m/z 491 (MH⁺). ¹HNMR (300 MHz, CDCl₃) δ8.67 (s, 1H), 7.32 (s, 1H), 6.55 (s, 1H), 6.33 (d, J=5.1 Hz, 1H), 4.03-3.95 (m, 4H), 3.88-3.84 (m, 4H), 3.05 (d, J=5.1 Hz, 3H), 2.89 (s, 6H), 1.95-1.92 (m, 1H), 0.99-0.93 (m, 2H), 0.87-0.84 (m, 2H).

### 1.5) Synthesis of Compound 78, 2-((3-cyclopropyl-1H-pyrazol-5-yl)amino)-N-methyl-4-morpholinofuro[3,2-d]pyrimidine-6-carboxamide hydrochloride

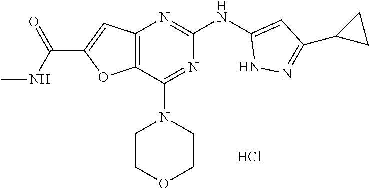 [SMILES: CNC(=O)c1cc2nc(Nc3cc(C4CC4)n[nH]3)nc(N3CCOCC3)c2o1 ; Cl]

To a solution of 2-((3-cyclopropyl-1-(N,N-dimethylsulfamoyl)-1H-pyrazol-5-yl)amino)-N-methyl-4-morpholinofuro[3,2-d]pyrimidine-6-carboxamide (62 mg, 0.13 mmol) in DCM (4 mL) was added HCl/Et₂O (3 mL). The reaction mixture was stirred at room temperature for 2 h. After concentration and slurry in MeOH/Et₂O (1/20, 2 mL), 2-((3-cyclopropyl-1H-pyrazol-5-yl)amino)-N-methyl-4-morpholinofuro[3,2-d]pyrimidine-6-carboxamide hydrochloride (Compound 78, 32.8 mg, 0.078 mmol) was obtained as a white solid. LC-MS (ESI+): m/z 384 (MH⁺). ¹HNMR (300 MHz, CD₃OD) δ 7.51 (s, 1H), 5.73 (s, 1H), 4.30-4.15 (m, 4H), 3.92-3.86 (m, 4H), 2.96 (s, 3H), 1.99-1.91 (m, 1H), 1.19-1.15 (m, 2H), 0.89-0.67 (m, 2H).

The compounds in Table 2 were prepared using methods analogous to those described in the referenced General Synthetic Routes.

TABLE 2

| | | | General | |
|---|---|---|---|---|
| Compound | | | Synthetic | |
| # | Name | Structure | Route | Data |
| 2 | 4-morpholino-N-[5- (o-tolyl)-1H-pyrazol- 3-yl]-6-(4- pyridyl)furo[3,2- d]pyrimidin-2-amine, hydrogen chloride | 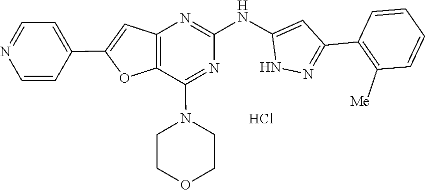 [SMILES: Cc1ccccc1-c1cc(Nc2nc(N3CCOCC3)c3oc(-c4ccncc4)cc3n2)[nH]n1 ; Cl] | 1 | LC-MS (ESI+): m/z 454 (MH⁺). ¹HNMR (300 MHz, CD₃OD) δ 8.98 (d, J = 6.9 Hz, 2H), 8.58 (d, J = 6.9 Hz, 2H), 8.08 (s, 1H), 7.43 (d, J = 6.3 Hz, 1H), 7.39-7.29 (m, 3H), 6.24 (s, 1H), 4.38-4.23 (m, 4H), 3.98-3.92 (m, 4H), 2.42 (s, 3H). |
| 3 | 4-morpholino-N-[5- (m-tolyl)-1H- pyrazol-3-yl]-6-(4- pyridyl)furo[3,2- d]pyrimidin-2-amine, hydrogen chloride | 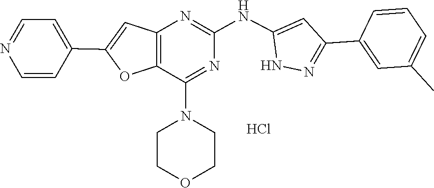 [SMILES: Cc1cccc(-c2cc(Nc3nc(N4CCOCC4)c4oc(-c5ccncc5)cc4n3)[nH]n2)c1 ; Cl] | 1 | LC-MS (ESI+): m/z 454 (MH⁺). ¹HNMR (300 MHz, CD₃OD) δ 8.92 (d, J = 6.3 Hz, 2H), 8.44 (d, J = 5.4 Hz, 2H), 7.98 (s, 1H), 7.60-7.50 (m, 2H), 7.39 (t, J = 7.6 Hz, 1H), 7.27 (d, J = 6.9 Hz, 1H), 6.42 (s, 1H), 4.40-4.22 (m, 4H), 3.94-3.88 (m, 4H), 2.43 (s, 3H). |
| 4 | 4-morpholino-N-[5- (p-tolyl)-1H-pyrazol- 3-yl]-6-(4- pyridyl)furo[3,2- d]pyrimidin-2-amine, hydrogen chloride | 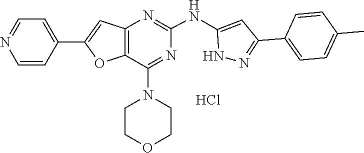 [SMILES: Cc1ccc(-c2cc(Nc3nc(N4CCOCC4)c4oc(-c5ccncc5)cc4n3)[nH]n2)cc1 ; Cl] | 1 | LC-MS (ESI+): m/z 454 (MH⁺). ¹HNMR (300 MHz, CD₃OD) δ 8.96 (d, J = 6.6 Hz, 2H), 8.53 (d, J = 6.9 Hz, 2H), 8.05 (s, 1H), 7.60 (d, J = 8.1 Hz, 2H), 7.31 (d, J = 8.1 Hz, 2H), 6.39 (s, 1H), 4.39-4.18 (m, 4H), |
| | | | | 3.99-3.91 (m, 4H), |
| | | | | 2.39 (s, 3H). |
| 5 | N-(5-methyl-1H- pyrazol-3-yl)-4- morpholino-6-(4- pyridyl)furo[3,2- d]pyrimidin-2-amine, hydrogen chloride | 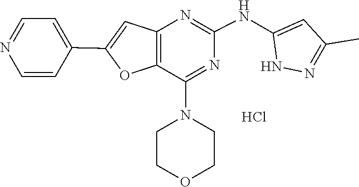 [SMILES: Cc1cc(Nc2nc(N3CCOCC3)c3oc(-c4ccncc4)cc3n2)[nH]n1 ; Cl] | 1 | LC-MS (ESI+): m/z 378 (MH⁺). ¹HNMR (300 MHz, CD₃OD) δ 8.98 (d, J = 6.9 Hz, 2H), 8.61 (d, J = 6.9 Hz, 2H), 8.07 (s, 1H), 5.93 (s, 1H), 4.36-4.24 (m, H), 3.94-3.91 (m, 4H), 2.35 (s, 3H). |
| 6 | 4-morpholino-6-(4- pyridyl)-N-[5-(4- pyridyl)-1H-pyrazol- 3-yl]furo[3,2- d]pyrimidin-2-amine, hydrogen chloride | 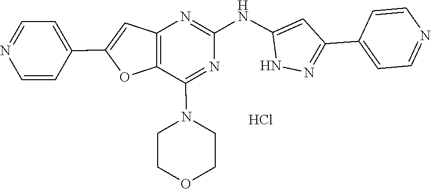 [SMILES: c1cc(-c2cc(Nc3nc(N4CCOCC4)c4oc(-c5ccncc5)cc4n3)[nH]n2)ccn1 ; Cl] | 1 | LC-MS (ESI+): m/z 441 (MH⁺). ¹HNMR (300 MHz, CD₃OD) δ 8.97 (d, J = 6.9 Hz, 2H), 8.91 (d, J = 6.9 Hz, 2H), 8.56 (d, J = 6.6 Hz, 2H), 8.44 (d, J = 6.9 Hz, 2H), 8.05 (s, 1H), 7.06 (s, 1H), 4.29-4.22 (m, 4H), 3.96-3.92 (m, 4H). |
| 7 | N-[5-(3- fluorophenyl)- 1H- pyrazol-3-yl]-4- morpholino-furo[3,2- d]pyrimidin-2-amine, hydrogen chloride | 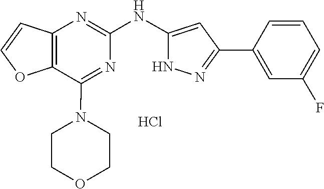 [SMILES: Fc1cccc(-c2cc(Nc3nc(N4CCOCC4)c4occc4n3)[nH]n2)c1 ; Cl] | 2 | LC-MS (ESI+): m/z 381 (MH⁺). ¹HNMR (300 MHz, CD₃OD) δ 8.15 (d, J = 2.1 Hz, 1H), 7.60-7.47 (m, 3H), 7.20-7.14 (m, 1H), 7.07 (d, J = 2.1 Hz, 1H), 6.46 (s, 1H), 4.27-4.13 (m, 4H), 3.89-3.86 (m, 4H). |
| 8 | 4-morpholino-N-[5- (m-tolyl)-1H- pyrazol-3- yl]furo[3,2- d]pyrimidin-2-amine, hydrogen chloride | 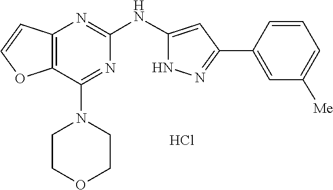 [SMILES: Cc1cccc(-c2cc(Nc3nc(N4CCOCC4)c4occc4n3)[nH]n2)c1 ; Cl] | 2 | LC-MS (ESI+): m/z 377 (MH⁺). ¹HNMR (300 MHz, CD₃OD) δ 8.15 (d, J = 2.4 Hz, 1H), 7.54 (s, 1H), 7.50 (d, J = 8.1 Hz, 1H), 7.36 (t, J = 7.5 Hz, 1H), 7.25 (d, J = 7.5 Hz, 1H), 7.07 (d, J = 2.1 Hz, 1H), 6.4 (s, 1H), 4.22-4.17 (m, 4H), 3.89-3.86 (m, |
| | | | | 4H), 2.42 (s, 3H). |
| 9 | N-(5-methyl-1H- pyrazol-3-yl)-4- morpholino-furo[3,2- d]pyrimidin-2-amine, hydrogen chloride | 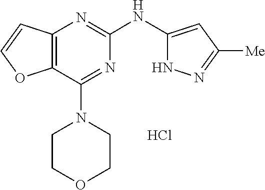 [SMILES: Cc1cc(Nc2nc(N3CCOCC3)c3occc3n2)[nH]n1 ; Cl] | 2 | LC-MS (ESI+): m/z 301 (MH⁺). ¹HNMR (300 MHz, CD₃OD) δ 8.12 (d, J = 2.1 Hz, 1H), 7.05 (d, J = 2.1 Hz, 1H), 5.88 (s, 1H), 4.22-4.12 (m, 4H), 3.92-3.84 (m, 4H), 2.33 (s, 3H). |
| 13 | 4-morpholino-2-((3- phenyl-1H-pyrazol-5- yl)methyl)-6- (piperidin-1- ylmethyl)furo[3,2- d]pyrimidine, hydrogen chloride | 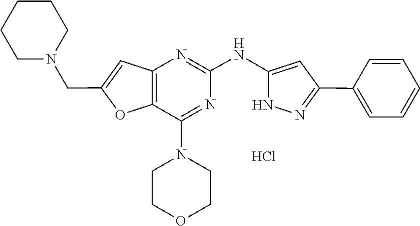 [SMILES: c1ccc(-c2cc(Nc3nc(N4CCOCC4)c4oc(CN5CCCCC5)cc4n3)[nH]n2)cc1 ; Cl] | 4 | LC-MS (ESI+): m/z 460 (MH⁺). ¹HNMR (300 MHz, DMSO-d₆) δ 11.81 (s, 1H), 11.03 (s, 1H), 7.82-7.79 (m, 2H), 7.52-7.47 (m, 2H), 7.41 (d, J = 7.2 Hz, 1H), 7.37 (s, 1H), 6.61 (s, 1H), 4.58-4.56 (m, 2H), 4.22-4.15 (m, 4H), 3.90-3.82 (m, 4H), 3.39-3.71 (m, 2H), 2.95-2.79 (m, 2H), 1.90-1.68 (m, 5H), 1.38-1.34 (m, 1H). |
| 14 | N,N-dimethyl-1-(4- morpholino-2-((3- phenyl-1H-pyrazol-5- yl)methyl)furo[3,2- d]pyrimidin-6- yl)methanamine, hydrogen chloride | 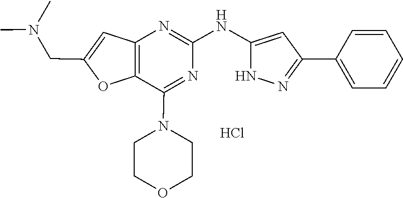 [SMILES: CN(C)Cc1cc2nc(Nc3cc(-c4ccccc4)n[nH]3)nc(N3CCOCC3)c2o1 ; Cl] | 4 | LC-MS (ESI+): m/z 420 (MH⁺). ¹HNMR (300 MHz, CD₃OD) δ 7.71 (d, J = 6.9 Hz, 2H), 7.52-7.43 (m, 3H), 7.36 (s, 1H), 6.42 (s, 1H), 4.68 (s, 2H), 4.25-4.17 (m, 4H), 3.91-3.87 (m, 4H), 2.96 (s, 6H). |
| 15 | 6-(4- methylpiperazin-1- yl)methyl)-4- morpholino-2-((3- phenyl-1H-pyrazol-5- yl)methyl)furo[3,2- d]pyrimidine, hydrogen chloride | 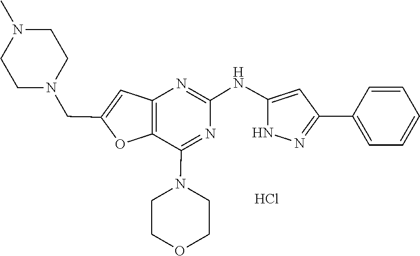 [SMILES: CN1CCN(Cc2cc3nc(Nc4cc(-c5ccccc5)n[nH]4)nc(N4CCOCC4)c3o2)CC1 ; Cl] | 4 | LC-MS (ESI+): m/z 475 (MH⁺). ¹HNMR (300 MHz, CD₃OD) δ 7.71 (d, J = 6.9 Hz, 2H), 7.51-7.40 (m, 3H), 7.09 (s, 1H), 6.41 (s, 1H), 4.22-4.17 (m, 4H), 4.09 (s, 2H), 3.89-3.86 (m, 4H), 3.59-3.45 (m, 4H), 2.99-2.73 (m, 7H). |
| 16 | 4-morpholino-2-((3- phenyl-1H-pyrazol-5- yl)methyl)-6- (pyrrolidin-1- ylmethyl)furo[3,2- d]pyrimidine, hydrogen chloride | 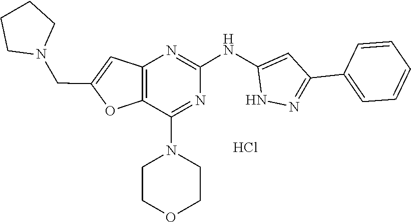 [SMILES: c1ccc(-c2cc(Nc3nc(N4CCOCC4)c4oc(CN5CCCC5)cc4n3)[nH]n2)cc1 ; Cl] | 4 | LC-MS (ESI+): m/z 446 (MH⁺). ¹HNMR (300 MHz, DMSO-d₆) δ 7.81 (d, J = 7.2 Hz, 2H), 7.52-7.47 (m, 2H), 7.43-7.38 (m, 1H), 7.33 (s, 1H), 6.61 (s, 1H), 4.69-4.67 (m, 2H), 4.29-4.15 (m, 4H), 3.92-3.81 (m, 4H), 3.47-3.37 (m, 2H), 3.21-3.10 (m, 2H), 2.02-1.94 (m, 4H). |
| 17 | 2-methoxy-N- methyl-N-((4- morpholino-2-((3- phenyl-1H-pyrazol-5- yl)methyl)furo[3,2- d]pyrimidin-6- yl)methyl)ethan-1- amine hydrogen chloride | 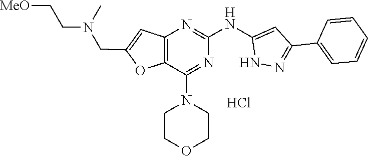 [SMILES: COCCN(C)Cc1cc2nc(Nc3cc(-c4ccccc4)n[nH]3)nc(N3CCOCC3)c2o1 ; Cl] | 4 | LC-MS (ESI+): m/z 464 (MH⁺). ¹HNMR (300 MHz, CD₃OD) δ 7.71 (d, J = 6.9 Hz, 2H), 7.52-7.40 (m, 3H), 7.38 (s, 1H), 6.42 (s, 1H), 4.74 (brs, 2H), 4.29-4.22 (m, 4H), 3.91-3.88 (m, 4H), 3.81-3.78 (m, 2H), |
| | | | | 3.59-3.32 (m, 5H), |
| | | | | 3.07 (s, 3H). |
| 18 | 4-morpholino-6-(4- pyridyl)-N-[5-(3- pyridyl)-1H-pyrazol- 3-yl]furo[3,2- d]pyrimidin-2-amine, hydrogen chloride | 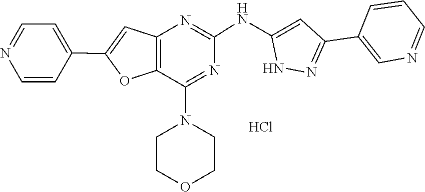 [SMILES: c1cncc(-c2cc(Nc3nc(N4CCOCC4)c4oc(-c5ccncc5)cc4n3)[nH]n2)c1 ; Cl] | 5 | LC-MS (ESI+): m/z 441 (MH⁺). ¹HNMR (300 MHz, CD₃OD) δ 9.26 (d, J = 7.8 Hz, 1H), 8.98 (d, J = 7.5 Hz, 2H), 8.83 (d, 7 = 6.3 Hz, 2H), 8.53 (d, J = 6.6 Hz, 2H), 8.09 (t, J = 6.9 Hz, 1H), 8.04 (s, 1H), 6.79 (s, 1H), 4.66-4.33 (m, 4H), 3.96-3.94 (m, 4H). |
| 19 | 4-morpholino-6-(4- pyridyl)-N-[5-(2- pyridyl)-1H-pyrazol- 3-yl]furo[3,2- d]pyrimidin-2-amine, hydrogen chloride | 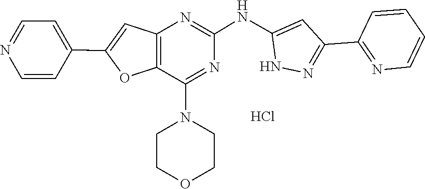 [SMILES: c1ccc(-c2cc(Nc3nc(N4CCOCC4)c4oc(-c5ccncc5)cc4n3)[nH]n2)nc1 ; Cl] | 5 | LC-MS (ESI+): m/z 441 (MH⁺). ¹HNMR (300 MHz, CD₃OD) δ 8.97 (d, J = 6.9 Hz, 2H), 8.70 (d, J = 4.8 Hz, 1H), 8.57 (d, J = 6.9 Hz, 2H), 8.18 (t, J = 7.5 Hz, 1H), 8.09- 8.04 (m, 2H), 7.61 (t, J = 6.0 Hz, 1H), 6.78 (s, 1H), 4.66-4.33 (m, 4H), 3.96-3.94 (m, 4H). |
| 20 | N-[5-(6-methyl-2- pyridyl)-1H-pyrazol- 3-yl]-4-morpholino- 6-(4- pyridyl)furo[3,2- d]pyrimidin-2-amine, hydrogen chloride | 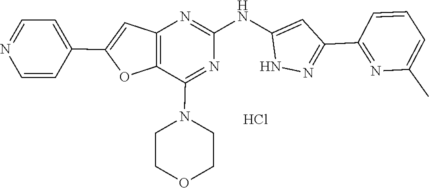 [SMILES: Cc1cccc(-c2cc(Nc3nc(N4CCOCC4)c4oc(-c5ccncc5)cc4n3)[nH]n2)n1 ; Cl] | 5 | LC-MS (ESI+): m/z 455 (MH⁺). ¹HNMR (300 MHz, DMSO-d₆) δ 10.77 (s, 1H), 8.92 (d, J = 6.3 Hz, 2H), 8.31 (d, J = 6.3 Hz, 2H), 8.03 (t, J = 7.8 Hz, 1H), 7.97 (s, 1H), 7.91 (d, J = 7.8 Hz, 1H), 7.44 (d, J = 7.8 Hz, 1H), 6.91 (s, 1H), 4.25-4.14 (m, 4H), |
| | | | | 3.97-3.86 (m, 4H), |
| | | | | 2.64 (s, 3H). |
| 21 | N-[5-(4-methyl-2- pyridyl)-1H-pyrazol- 3-yl]-4-morpholino- 6-(4- pyridyl)fnro[3,2- d]pyrimidin-2-amine, hydrogen chloride | 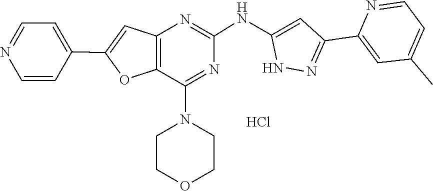 [SMILES: Cc1ccnc(-c2cc(Nc3nc(N4CCOCC4)c4oc(-c5ccncc5)cc4n3)[nH]n2)c1 ; Cl] | 5 | LC-MS (ESI+): m/z 455 (MH⁺). ¹HNMR (300 MHz, CD₃OD) δ 8.94 (d, J = 6.6 Hz, 2H), 8.55-8.50 (m, 3H), 8.06 (s, 1H), 7.96 (s, 1H), 7.60 (d, J = 5.1 Hz, 1H), 6.79 (s, 1H), 4.24-4.20 (m, 4H), 3.93-3.90 (m, 4H), 2.63 (s, 3H). |
| 23 | N-[5-(5-methyl-3- pyridyl)-1H-pyrazol- 3-yl]-4-morpholino- 6-(4- pyridyl)furo[3,2- d]pyrimidin-2-amine, hydrogen chloride | 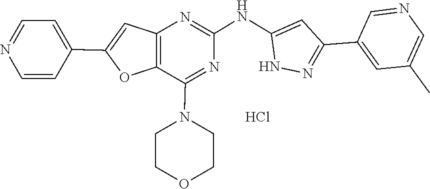 [SMILES: Cc1cncc(-c2cc(Nc3nc(N4CCOCC4)c4oc(-c5ccncc5)cc4n3)[nH]n2)c1 ; Cl] | 5 | LC-MS (ESI+): m/z 455 (MH+). 1HNMR (300 MHz, CD3OD) δ 9.09 (s, 1H), 8.95 (d, J = 6.6 Hz, 2H), 8.76 (s, 1H), 8.72 (s, 1H), 8.51 (d, J = 6.9 Hz, 2H), 8.03 (s, 1H), 6.79 (s, 1H), 4.29-4.18 (m, 4H), 3.94-3.91 (m, 4H), 2.64 (s, 3H). |
| 24 | N-[5-(3- methoxypheny1)-1H- pyrazol-3-yl]-4- morpholino-6-(4- pyridyl)furo[3,2- d]pyrimidin-2-amine, hydrogen chloride | 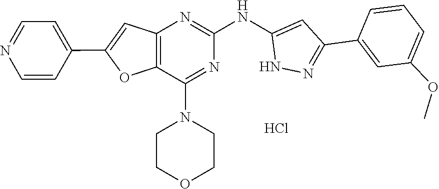 [SMILES: COc1cccc(-c2cc(Nc3nc(N4CCOCC4)c4oc(-c5ccncc5)cc4n3)[nH]n2)c1 ; Cl] | 5 | LC-MS (ESI+): m/z 470 (MH⁺). ¹HNMR (300 MHz, CD₃OD) δ 8.95 (d, J = 6.6 Hz, 2H), 8.52 (d, J = 6.6 Hz, 2H), 8.05 (s, 1H), 7.40 (t, J = 7.8 Hz, 1H), 7.28 (d, J = 7.5 Hz, 2H),6.99 (d, J = 6.6 Hz, 1H), 6.44 (s, 1H), 4.37-4.13 (m, 4H), 4.02-3.92 (m, 4H), 3.87 (s, 3H). |
| 25 | 4-morpholino-6-(4- pyridyl)-N-[5-[3- (trifluoromethoxy) phenyl]-1H-pyrazol-3- yl]furo[3,2- d]pyrimidin-2-amine, hydrogen chloride | 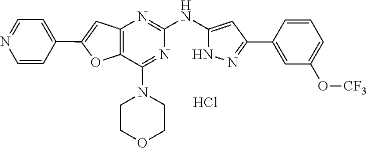 [SMILES: FC(F)(F)Oc1cccc(-c2cc(Nc3nc(N4CCOCC4)c4oc(-c5ccncc5)cc4n3)[nH]n2)c1 ; Cl] | 5 | LC-MS (ESI+): m/z 524 (MH⁺). ¹HNMR (300 MHz, CD₃OD) δ 8.93 (d, J = 6.6 Hz, 2H), 8.45 (d, J = 6.9 Hz, 2H), 7.99 (s, 1H), 7.75 (d, J = 7.2 Hz, 1H), 7.67 (s,1H), 7.61 (t, J = 8.1 Hz, 1H), 7.32 (d, J = 8.7 Hz, |
| | | | | 1H), 6.53 (s,1H), 4.40- |
| | | | | 4.24 (m, 4H), 3.96- |
| | | | | 3.92 (m, 4H). |
| 26 | 4-morpholino-N-(5- phenyl-1H-pyrazol-3- yl)-6-(2- pyridyl)furo[3,2- d]pyrimidin-2-amine, hydrogen chloride | 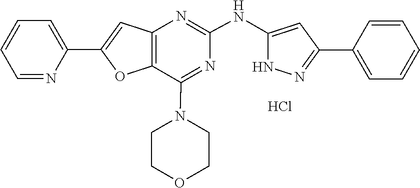 [SMILES: c1ccc(-c2cc(Nc3nc(N4CCOCC4)c4oc(-c5ccccn5)cc4n3)[nH]n2)cc1 ; Cl] | 6 | LC-MS (ESI+): m/z 440 (MH⁺). ¹HNMR (300 MHz, CD₃OD) δ 8.73 (d, J = 4.2 Hz, 1H), 8.09 (d, J = 7.8 Hz, 1H), 8.04-7.99 (m, 1H), 7.72 (d, J = 6.9 Hz, 2H), 7.59 (S,1H), 7.52-7.40 (m, 4H), 6.42 (s,1H), 4.47- 4.19 (m, 4H), 3.96- 3.83 (m, 4H). |
| 27 | 4-morpholino-N-[5- (m-tolyl)-1H- pyrazol-3-yl]-6-(2- pyridyl)furo[3,2- d]pyrimidin-2-amine, hydrogen chloride | 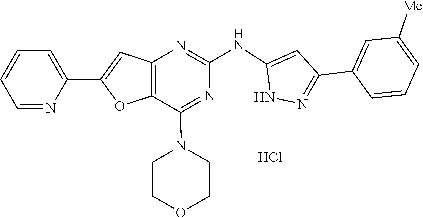 [SMILES: Cc1cccc(-c2cc(Nc3nc(N4CCOCC4)c4oc(-c5ccccn5)cc4n3)[nH]n2)c1 ; Cl] | 6 | LC-MS (ESI+): m/z 454 (MH⁺). ¹HNMR (300 MHz, CD₃OD) δ 8.73 (d, J = 4.5 Hz, 1H), 8.13-8.02 (m, 2H), 7.71 (s, 1H), 7.57-7.49 (m, 3H), 7.37 (t, J = 7.5 Hz, 1H), 7.25 (d, J = 7.2 Hz, 1H),6.4O (s,1H), 4.45-4.12 (m, 4H), 3.97-3.85 (m, 4H), 2.42 (s, 3H). |
| 29 | 4-morpholino-N-(5- phenyl-1H-pyrazol-3- yl)-6-(3- pyridyl)furo[3,2- d]pyrimidin-2-amine, hydrogen chloride | 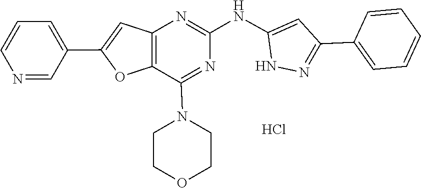 [SMILES: c1ccc(-c2cc(Nc3nc(N4CCOCC4)c4oc(-c5cccnc5)cc4n3)[nH]n2)cc1 ; Cl] | 6 | LC-MS (ESI+): m/z 440 (MH⁺). ¹HNMR (300 MHz, CD₃OD) δ 9.52 (s, 1H), 9.08 (d, J = 8.4 Hz, 1H), 8.92 (d, J = 5.1 Hz, 1H), 8.21- 8.16 (m, 1H), 7.83 (s, 1H), 7.72 (d, J = 6.9 Hz, 2H), 7.54-7.40 (m, 3H), 6.44 (s,1H), 4.40- 4.11 (m, 4H), 3.97- 3.88 (m, 4H). |
| 30 | 4-morpholino-N-[5- (m-tolyl)-1H- pyrazol-3-yl]-6-(3- pyridyl)furo[3,2- d]pyrimidin-2-amine, hydrogen chloride | 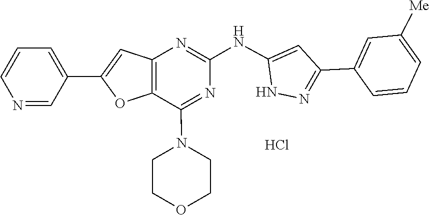 [SMILES: Cc1cccc(-c2cc(Nc3nc(N4CCOCC4)c4oc(-c5cccnc5)cc4n3)[nH]n2)c1 ; Cl] | 6 | LC-MS (ESI+): m/z 454 (MH⁺). ¹HNMR (300 MHz, CD₃OD) δ 9.38 (s, 1H), 8.84 (d, J = 6.0 Hz, 2H), 8.01- 7.97 (m, 1H), 7.73 (s, 1H), 7.55-7.50 (m, 2H), 7.37 (t, J = 7.5 Hz, 1H), 7.25 (d, J = 7.5 Hz, 1H), 6.41 (s,1H), 4.40-4.11 (m, 4H), 3.96-3.85 (m, 4H), 2.42 (s, 3H). |
| 31 | 4-morpholino-6-(3- pyridyl)-N-[5-(4- pyridyl)-1H-pyrazol- 3-yl]furo[3,2- d]pyrimidin-2-amine, hydrogen chloride | 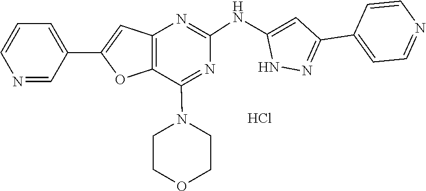 [SMILES: c1cncc(-c2cc3nc(Nc4cc(-c5ccncc5)n[nH]4)nc(N4CCOCC4)c3o2)c1 ; Cl] | 6 | LC-MS (ESI+): m/z 441 (MH⁺). ¹HNMR (300 MHz, CDCl₃) δ 9.08 (s, 1H), 8.67-8.64 (m, 3H), 8.05 (d, J = 7.8, 1H), 7.70 (s, 2H), 7.60 (d, J = 5.1, 1H), 7.44 (d, J = 5.4 Hz, 1H), 7.01 (s, 1H), 6.15 (S,1H), 4.15-4.05 (m, 4H), 3.96-3.89 (m, 4H). |
| 32 | 6-(1-methylpiperidin- 4-yl)-4-morpholino- 2-((3-phenyl-1H- pyrazol-5- yl)methyl)furo[3,2- d]pyrimidine, hydrogen chloride | 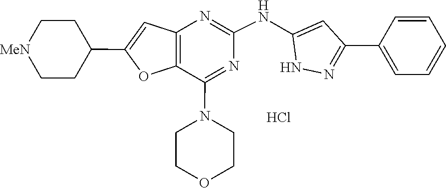 [SMILES: CN1CCC(c2cc3nc(Nc4cc(-c5ccccc5)n[nH]4)nc(N4CCOCC4)c3o2)CC1 ; Cl] | 6 (1.1) and 7 | LC-MS (ESI+): m/z 460 (MH⁺). ¹HNMR (300 MHz, CD₃OD) δ 7.71 (d, J = 6.9 Hz, 2H), 7.51-7.39 (m, 3H), 6.89 (s, 1H), 6.42 (s, 1H), 4.25-4.10 (m, 4H), 3.96-3.89 (m, 4H), 3.69-3.60 (m, 2H), 3.30-3.17(m, 3H), 2.93 (s, 3H), 2.44-2.30 (m, 2H), 2.15-2.02 (m, 2H). |
| 33 | 6-(1-methylpiperidin- 4-yl)-4-morpholino- 2-((3-(m-tolyl)-1H- pyrazol-5- yl)methyl)furo[3,2- d]pyrimidine, hydrogen chloride | 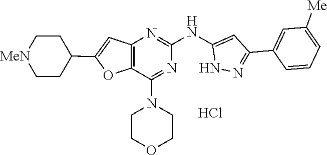 [SMILES: Cc1cccc(-c2cc(Nc3nc(N4CCOCC4)c4oc(C5CCN(C)CC5)cc4n3)[nH]n2)c1 ; Cl] | 6 (1.1) and 7 | LC-MS (ESI+): m/z 474 (MH⁺). ¹HNMR (300 MHz, CD₃OD) δ 7.54 (s, 1H), 7.50 (d, J = 7.5 Hz, 1H), 7.36 (t, J = 7.8 Hz, 1H), 7.25 (d, J = 7.5 Hz, 1H), 6.89 (s, 1H), 6.42 (s, 1H), 4.25-4.10 (m, 4H), 3.90-3.87 (m, |
| | | | | 4H), 3.69-3.59 (m, |
| | | | | 2H), 3.30-3.17(m, |
| | | | | 3H), 2.94 (s, 3H), |
| | | | | 2.44-2.35 (m, 5H), |
| | | | | 2.18-2.02 (m, 2H). |
| 36 | 6-(1-methylpiperidin- 3-yl)-4-morpholino- 2-((3-(m-tolyl)-1H- pyrazol-5- yl)methyl)furo[3,2- d]pyrimidine, hydrogen chloride | 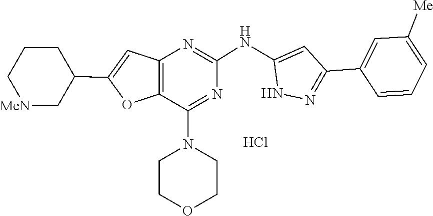 [SMILES: Cc1cccc(-c2cc(Nc3nc(N4CCOCC4)c4oc(C5CCCN(C)C5)cc4n3)[nH]n2)c1 ; Cl] | 6 (1.1) and 8 | LC-MS (ESI+): m/z 474 (MH⁺). ¹HNMR (300 MHz, CD₃OD) δ 7.54 (s, 1H), 7.50 (d, J = 7.5 Hz, 1H), 7.36 (t, J = 7.5 Hz, 1H), 7.25 (d, J = 7.5 Hz, 1H), 6.95 (s, 1H), 6.39 (s, 1H), 4.25-4.10 (m, 4H), 3.96-3.85 (m, 4H), 3.62-3.45 (m, 3H), 3.30-3.21(m, 1H), 3.07-2.98(m, |
| | | | | 1H), 2.97 (s, 3H), 2.42 |
| | | | | (s, 3H), 2.32-2.88 (m, |
| | | | | 1H), 2.19-2.14 (m, |
| | | | | 1H), 2.10-2.02 |
| | | | | (m, 1H), 1.85-1.80 |
| | | | | (m, 1H). |
| 37 | 6-(1-methylpiperidin- 3-yl)-4-morpholino- 2-((3-(pyridin-4-yl)- 1H-pyrazol-5- yl)methyl)furo[3,2- d]pyrimidine, hydrogen chloride | 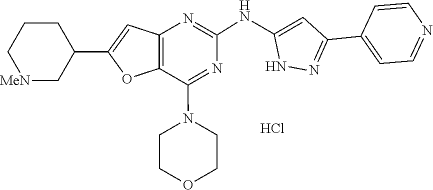 [SMILES: CN1CCCC(c2cc3nc(Nc4cc(-c5ccncc5)n[nH]4)nc(N4CCOCC4)c3o2)C1 ; Cl] | 6 (1.1) and 8 | LC-MS (ESI+): m/z 461 (MH⁺). ¹HNMR (300 MHz, CD₃OD) δ 8.91 (d, J = 6.3 Hz, 2H), 8.45 (d, J = 6.6 Hz, 2H), 7.06 (s, 1H), 6.95 (s, 1H), 4.25-4.10 (m, 4H), 3.96-3.85 (m, 4H), 3.61-3.44 (m, 2H), 3.30-3.21(m, 2H), 3.14-3.06 (m, 1H), 2.97 (s, 3H), |
| | | | | 2.31-2.27 (m, 1H), |
| | | | | 2.17-2.04 (m, 2H), |
| | | | | 1.95-1.76 (m, 1H). |
| 38 | 6-(1- methylpyrrolidin-3- yl)-4-morpholino-2- ((3-phenyl-1H- pyrazol-5- yl)methyl)furo[3,2- d]pyrimidine, hydrogen chloride | 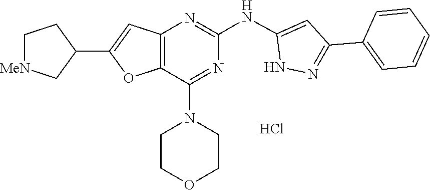 [SMILES: CN1CCC(c2cc3nc(Nc4cc(-c5ccccc5)n[nH]4)nc(N4CCOCC4)c3o2)C1 ; Cl] | 6 (1.1) and 9 | LC-MS (ESI+): m/z 446 (MH⁺). ¹HNMR (300 MHz, CD₃OD) δ 7.71 (d, J = 7.2 Hz, 2H), 7.51-7.39 (m, 3H), 7.03 (s, 1H), 6.41 (s, 1H), 4.30-4.10 (m, 4H), 4.08-4.02 (m, 1H), 3.96-3.88 (m, 4H), 3.66-3.54 (m, 1H), 3.49-3.42 (m, 3H), 3.05 (d, J = 9.6 |
| | | | | Hz, 3H), 2.71-2.40 (m, |
| | | | | 2H). |
| 40 | 6-(1- methylpyrrolidin-3- yl)-4-morpholino-2- ((3-(pyridin-4-yl)- 1H-pyrazol-5- yl)methyl)furo[3,2- d]pyrimidine, hydrogen chloride | 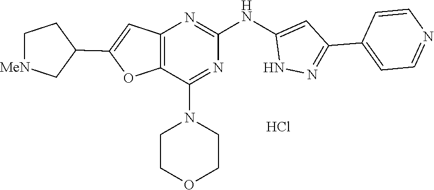 [SMILES: CN1CCC(c2cc3nc(Nc4cc(-c5ccncc5)n[nH]4)nc(N4CCOCC4)c3o2)C1 ; Cl] | 6 (1.1) and 9 | LC-MS (ESI+): m/z 447 (MH⁺). ¹HNMR (300 MHz, D₂O) δ 8.70 (d, J = 6.9 Hz, 2H), 8.20 (d, J = 6.9 Hz, 2H), 6.91 (s, 1H), 6.74 (d, J = 3.3 Hz, 1H), 4.21-4.15 (m, 4H), 3.94-3.72 (m, 6H), 3.61-3.49 (m, 1H), 3.32-3.20 (m, 2H), 2.93 (d, J = 5.7 |
| | | | | Hz, 3H), 2.61-2.23 (m, |
| | | | | 2H). |
| 41 | 4-morpholino-2-(4- phenylpyrazol-1-yl)- 6-(4- pyridyl)furo[3,2- d]pyrimidine, hydrogen chloride | 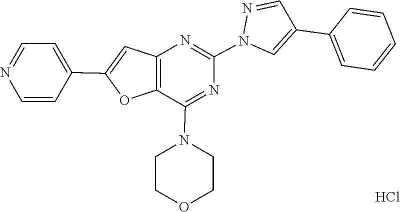 [SMILES: c1ccc(-c2cnn(-c3nc(N4CCOCC4)c4oc(-c5ccncc5)cc4n3)c2)cc1 ; Cl] | 10 | LC-MS (ESI+): m/z 425 (MH⁺). ¹HNMR (300 MHz, CDCl₃) δ 8.77 (d, J = 5.7 Hz, 2H), 8.73 (s, 1H), 8.08 (s, 1H), 7.68 (d, J = 6.0 Hz, 2H), 7.62 (d, J = 6.0 Hz, 2H), 7.41 (t, J = 7.5 Hz, 2H), 7.32- 7.26 (m, 2H), 4.21- 4.16 (m, 4H), 3.97- 3.91 (m, 4H). |
| 45 | 4-morpholino-N-(4- phenyloxazol-2-yl)- 6-(4- pyridyl)furo[3,2- d]pyrimidin-2-amine, hydrogen chloride | 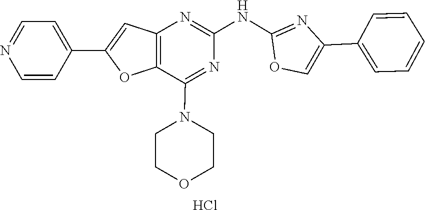 [SMILES: c1ccc(-c2coc(Nc3nc(N4CCOCC4)c4oc(-c5ccncc5)cc4n3)n2)cc1 ; Cl] | 12 | LC-MS (ESI+): m/z 441 (MH⁺). ¹HNMR (300 MHz, DMSO-d₆) δ 10.41 (s, 1H), 8.74 (d, J = 6.3 Hz, 2H), 8.29 (s, 1H), 7.93 (d, J = 6.3 Hz, 2H), 7.75 (d, J = 7.2 Hz, 2H), 7.69 (s, 1H), 7.45-7.40 (m, 2H), 7.32-7.30 (m, 1H), 4.08-4.03 (m, 4H), 3.85-3.78 (m, 4H). |
| 46 | 4-morpholino-N-(5- phenyloxazol-2-yl)- 6-(4- pyridyl)furo[3,2- d]pyrimidin-2-amine, hydrogen chloride | 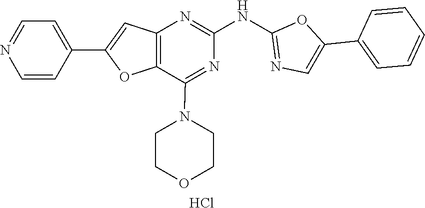 [SMILES: c1ccc(-c2cnc(Nc3nc(N4CCOCC4)c4oc(-c5ccncc5)cc4n3)o2)cc1 ; Cl] | 12 | LC-MS (ESI+): m/z 441 (MH⁺). ¹HNMR (300 MHz, DMSO-d₆) δ 10.47 (s, 1H), 8.74 (d, J = 6.0 Hz, 2H), 7.94 (d, J = 6.0 Hz, 2H), 7.68 (s, 1H), 7.62 (d, J = 7.5 Hz, 2H), 7.51 (s, 1H), 7.48-7.43 (m, 2H), 7.32-7.27 (m, 1H), 4.08-4.03 (m, 4H), 3.83-3.76 (m, 4H). |
| 47 | 4-morpholino-N-(5- phenyl-1,3,4- oxadiazol-2-yl)-6-(4- pyridyl)furo[3,2- d]pyrimidin-2-amine, hydrogen chloride | 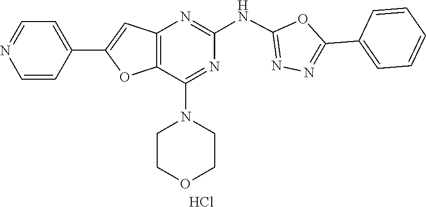 [SMILES: c1ccc(-c2nnc(Nc3nc(N4CCOCC4)c4oc(-c5ccncc5)cc4n3)o2)cc1 ; Cl] | 12 | LC-MS (ESI+): m/z 441 (MH⁺). ¹HNMR (300 MHz, DMSO-d₆) δ 10.97 (s, 1H), 8.74 (d, J = 6.3 Hz, 2H), 7.96-7.90 (m, 4H), 7.12 (s, 1H), 7.61-7.59 (m, 3H), 4.10-4.03 (m, 4H), 3.85-3.78 (m, 4H). |
| 48 | 4-morpholino-N-(1- phenylpyrazol-3-yl)- 6-(4- pyridyl)furo[3,2- d]pyrimidin-2-amine, hydrogen chloride | 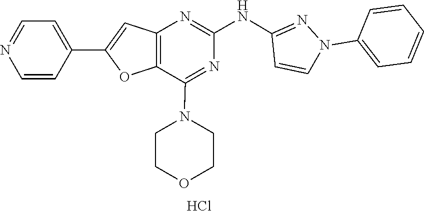 [SMILES: c1ccc(-n2ccc(Nc3nc(N4CCOCC4)c4oc(-c5ccncc5)cc4n3)n2)cc1 ; Cl] | 12 | LC-MS (ESI+): m/z 440 (MH⁺). ¹HNMR (300 MHz, CDCl₃) δ 8.73 (d, J = 6.0 Hz, 2H), 7.86 (d, J = 2.1 Hz, 1H), 7.65-7.61 (m, 4H), 7.46-7.41 (m, 3H), 7.23 (s, 1H), 7.14 (d, J = 2.1 Hz, 1H), 4.10-4.03 (m, 4H), 3.92-3.87 (m, 4H). |
| 49 | 4-morpholino-N-(1- phenylpyrazol-4-yl)- 6-(4- pyridyl)furo[3,2- d]pyrimidin-2-amine, hydrogen chloride | 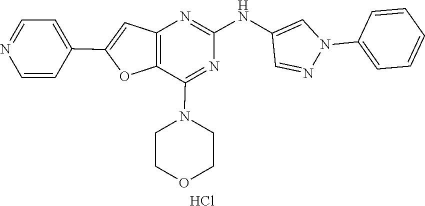 [SMILES: c1ccc(-n2cc(Nc3nc(N4CCOCC4)c4oc(-c5ccncc5)cc4n3)cn2)cc1 ; Cl] | 12 | LC-MS (ESI+): m/z 440 (MH⁺). ¹HNMR (300 MHz, CD₃OD) δ 8.65 (d, J = 6.6 Hz, 2H), 8.45 (s, 1H), 7.89 (d, J = 5.4 Hz, 2H), 7.82 (s, 1H), 7.71 (d, J = 7.8 Hz, 2H), 7.49 (t, J = 7.8 Hz, 2H), 7.37 (s, 1H), 7.30 (t, J = 7.5 Hz, 1H), 4.13-4.05 (m, 4H), 3.92-3.85 (m, 4H). |
| 51 | 4-morpholino-N- pyrazolo[1,5- a]pyridin-2-yl-6-(4- pyridyl)furo[3,2- d]pyrimidin-2-amine, hydrogen chloride | 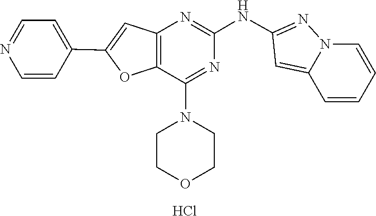 [SMILES: c1ccn2nc(Nc3nc(N4CCOCC4)c4oc(-c5ccncc5)cc4n3)cc2c1 ; Cl] | 12 | LC-MS (ESI+): m/z 414 (MH⁺). ¹HNMR (300 MHz, CDCl₃) δ 8.73 (d, J = 6.0 Hz, 2H), 8.28 (d, J = 7.2 Hz, 1H), 7.64 (d, J = 6.3 Hz, 2H), 7.52 (s, 1H), 7.41 (d, J = 9.0 Hz, 1H), 7.16 (s, 1H), 7.10-7.03 (m, 1H), 7.02 (s, 1H), 6.68-6.63 (m, 1H), 4.11-4.06 (m, 4H), 3.93-3.88 (m, 4H). |
| 54 | [4-morpholino-2-[(5- phenyl-1H-pyrazol-3- yl)amino]furo[3,2- d]pyrimidin-6-yl]- pyrrolidin-1-yl- methanone; hydrogen chloride | 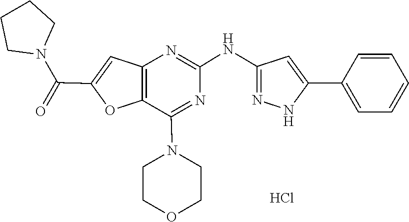 [SMILES: O=C(c1cc2nc(Nc3cc(-c4ccccc4)[nH]n3)nc(N3CCOCC3)c2o1)N1CCCC1 ; Cl] | 14 (1.1) and 15 | LC-MS (ESI+): m/z 460 (MH⁺). ¹HNMR (300 MHz, CD₃OD) δ 7.72 (d, J = 7.2 Hz, 2H), 7.52-7.42 (m, 4H), 6.42 (s, 1H), 4.28-4.20 (m, 4H), 3.90-3.78 (m, 6H), 3.69-3.64 (m, 2H), 2.10-2.03 (m, 2H), 2.01-1.97 (m, 2H). |
| 56 | N-ethyl-4 morpholino-2-[(5- phenyl-1H-pyrazol-3- yl)amino]furo[3,2- d]pyrimidine-6- carboxamide, hydrogen chloride | 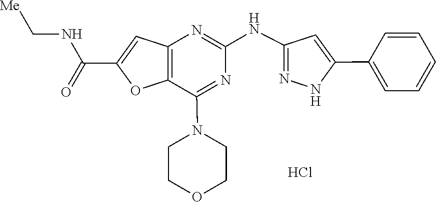 [SMILES: CCNC(=O)c1cc2nc(Nc3cc(-c4ccccc4)[nH]n3)nc(N3CCOCC3)c2o1 ; Cl] | 14 (1.1) and 15 | LC-MS (ESI+): m/z 434 (MH⁺). ¹HNMR (300 MHz, DMSO-d₆) δ 10.88 (s, 1H), 9.06- 9.05 (m, 1H), 7.80 (d, J = 7.5 Hz, 2H), 7.62- 7.42 (m, 3H), 7.40- 7.37 (m, 1H), 6.61 (s, 1H), 4.19-4.13 (m, 4H), 3.83-3.78 (m, 4H), 3.38-3.29 (m, 2H), 1.15 (t, J = 7.2 Hz, 3H). |
| 57 | N-ethyl-N-methyl-4- morpholino-2-[(5- phenyl-1H-pyrazol-3- yl)amino]furo[3,2- d]pyrimidine-6- carboxamide, hydrogen chloride | 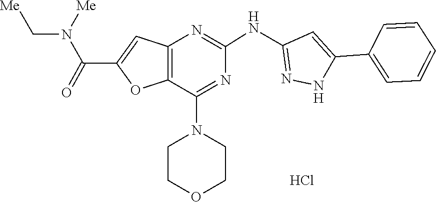 [SMILES: CCN(C)C(=O)c1cc2nc(Nc3cc(-c4ccccc4)[nH]n3)nc(N3CCOCC3)c2o1 ; Cl] | 14 (1.1) and 15 | LC-MS (ESH): m/z 448 (MH+). ¹HNMR (300 MHz, D₂O) δ 7.21-6.90 (m, 6H), 5.72 (s, 1H), 3.86-3.52 (m, 8H), 3.40-3.10 (m, 2H), 2.90 (s, 3H), 1.12-0.91 (m, 3H). |
| 58 | N-methoxy-4- morpholino-2-[(5- phenyl-1H-pyrazol-3- yl)amino]furo[3,2- d]pyrimidine-6- carboxamide, hydrogen chloride | 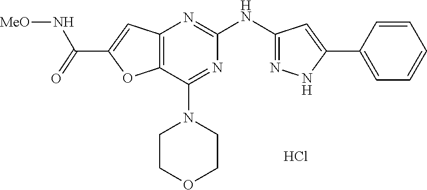 [SMILES: CONC(=O)c1cc2nc(Nc3cc(-c4ccccc4)[nH]n3)nc(N3CCOCC3)c2o1 ; Cl] | 14 (1.1) and 16 | LC-MS (ESI+): m/z 436 (MH⁺). ¹HNMR (300 MHz, CD₃OD) δ 7.71 (d, J = 7.2 Hz, 2H), 7.58 (s, 1H), 7.55-7.40 (m, 3H), 6.42 (s, 1H), 4.30-4.06 (m, 4H), 3.90-3.87 (m, 4H), 3.86 (s, 3H). |
| 59 | morpholino-2-[(5- phenyl-1H-pyrazol-3- yl)amino]furo[3,2- d]pyrimidine-6- carboxamide, hydrogen chloride | 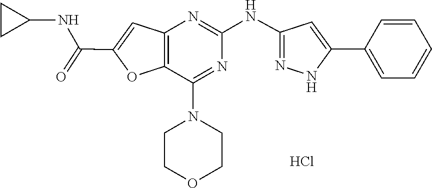 [SMILES: O=C(NC1CC1)c1cc2nc(Nc3cc(-c4ccccc4)[nH]n3)nc(N3CCOCC3)c2o1 ; Cl] | 14 (1.1) and 17 | LC-MS (ESI+): m/z 446 (MH⁺). ¹HNMR (300 MHz, CD₃OD) δ 7.71 (d, J = 7.2 Hz, 2H), 7.65 (s, 1H), 7.55-7.40 (m, 3H), 6.42 (s, 1H), 4.31-4.06 (m, 4H), 3.93-3.85 (m, 4H), 2.92-2.85 (m, 1H), 0.91-0.81 (m, 2H), 0.79-0.68 (m, 2H). |
| 60 | N-cyclopropyl-N- methyl-4- morpholino-2-[(5- phenyl-1H-pyrazol-3- yl)amino]furo[3,2- d]pyrimidine-6- carboxamide, hydrogen chloride | 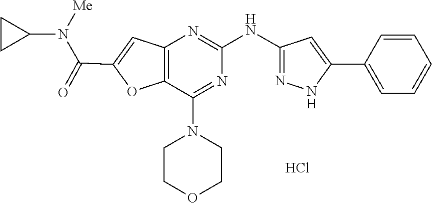 [SMILES: CN(C(=O)c1cc2nc(Nc3cc(-c4ccccc4)[nH]n3)nc(N3CCOCC3)c2o1)C1CC1 ; Cl] | 14 (1.1) and 17 | LC-MS (ESI+): m/z 460 (MH⁺). ¹HNMR (300 MHz, DMSO-d₆) δ 10.75 (s, 1H), 7.81 (d, J = 7.2 Hz, 2H), 7.50-7.45 (m, 3H), 7.40-7.35 (m, 1H), 6.62 (s, 1H), 4.12-4.03 (m, 4H), 3.84-3.76 (m, 4H), 3.20-3.10 (m, 1H), 3.03 (s, 3H), 0.81-0.75 (m, 2H), 0.69-0.61 (m, 2H). |
| 61 | N- (cyclopropylmethyl)- 4-morpholino-2-[(5- phenyl-1H-pyrazol-3- yl)amino]furo[3,2- d]pyrimidine-6- carboxamide, hydrogen chloride | 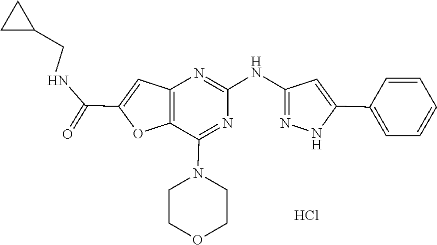 [SMILES: O=C(NCC1CC1)c1cc2nc(Nc3cc(-c4ccccc4)[nH]n3)nc(N3CCOCC3)c2o1 ; Cl] | 14 (1.1) and 17 | LC-MS (ESI+): m/z 460 (MH⁺). ¹HNMR (300 MHz, CD₃OD) δ 7.71 (d, J = 6.9 Hz, 2H), 7.56 (s, 1H), 7.51-7.40 (m, 3H), 6.42 (s, 1H), 4.38-4.09 (m, 4H), 3.96-3.88 (m, 4H), 3.34-3.20 (m, 2H), 1.20-1.13 (m, 1H), 0.58-0.52 (m, 2H), 0.34-0.29 (m, 2H). |
| 62 | N- (cyclopropylmethyl)- N-methyl-4- morpholino-2-[(5- phenyl-1H-pyrazol-3- yl)amino]furo[3,2- d]pyrimidine-6- carboxamide, hydrogen chloride | 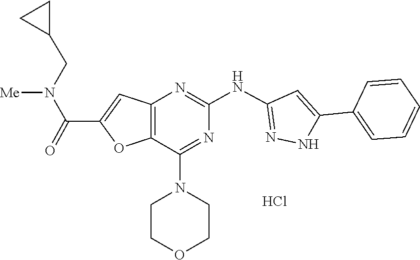 [SMILES: CN(CC1CC1)C(=O)c1cc2nc(Nc3cc(-c4ccccc4)[nH]n3)nc(N3CCOCC3)c2o1 ; Cl] | 14 (1.1) and 17 | LC-MS (ESI+): m/z 474 (MH⁺). ¹HNMR (300 MHz, CD₃OD) δ 7.72 (d, J = 6.9 Hz, 2H), 7.52-7.40 (m, 4H), 6.42 (s, 1H), 4.30-4.09 (m, 4H), 3.91-3.86 (m, 4H), 3.50-3.39 (m,2H), 3.34 (s, 1.5 H), 3.21 (s, 1.5 H), 1.20-1.13 (m, 1H), 0.66-0.58 (m, 2H), 0.38-0.31 (m, 2H). |
| 63 | 4-morpholino-2-[(5- phenyl-1H-pyrazol-3- yl)amino]-N-[(1S)-1- cyclopropylethyl]furo [3,2-d]pyrimidine-6- carboxamide, hydrogen chloride | 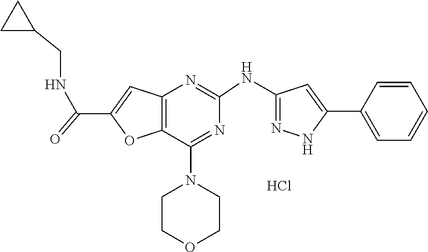 [SMILES: O=C(NCC1CC1)c1cc2nc(Nc3cc(-c4ccccc4)[nH]n3)nc(N3CCOCC3)c2o1 ; Cl] | 14 (1.1) and 17 | LC-MS (ESI+): m/z 474 (MH⁺). ¹HNMR (300 MHz, DMSO-d₆) δ 10.80 (s, 1H), 8.86 (d, J = 8.1 Hz, 1H), 7.79 (d, J = 7.5 Hz, 2H), 7.54-7.42 (m, 3H), 7.39-7.37 (m, 1H), 6.61(s, 1H), 4.16- 4.05 (m, 4H), 3.86- 3.79 (m, 4H), 3.40- 3.30 (m, 1H), 1.28 (d, J = 6.6 Hz, 3H), 1.11- 1.04 (m, 1H), 0.49- 0.41 (m, 2H), 0.39- 0.26 (m, 2H). |
| 67 | azetidin-1-yl(4- morpholino-2-(4- phenyl-1H-pyrazol-1- yl)furo[3,2- d]pyrimidin-6- yl)methanone | 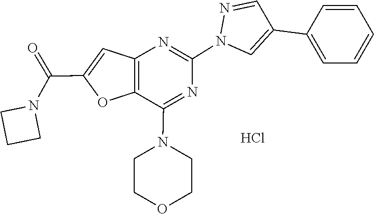 [SMILES: O=C(c1cc2nc(-n3cc(-c4ccccc4)cn3)nc(N3CCOCC3)c2o1)N1CCC1 ; Cl] | 14 (1.1) and 19 | LC-MS (ESI+): m/z 431 (MH⁺). ¹HNMR (300 MHz, CDCl₃) δ 8.72 (s, 1H), 8.09 (s, 1H), 7.61 (d, J = 7.5 Hz, 2H), 7.44-7.39 (m, 2H), 7.33-7.26 (m, 2H), 4.56 (t, J = 7.2 Hz, 2H), 4.28 (t, J = 7.5 Hz, 2H), 4.16-4.10 (m, 4H), 3.92-3.86 (m, 4H), 2.53-3.43 (m, 2H). |
| 68 | N-(5-cyclopropyl- 1H-pyrazol-3-yl)-4- morpholino-6- (pyridin-4- yl)furo[3,2- d]pyrimidin-2-amine, hydrogen chloride | 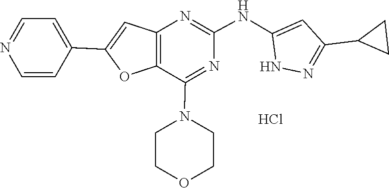 [SMILES: c1cc(-c2cc3nc(Nc4cc(C5CC5)n[nH]4)nc(N4CCOCC4)c3o2)ccn1 ; Cl] | 20 | LC-MS (ESI+): m/z 404 (MH₊). ¹HNMR (300 MHz, CD₃OD) δ 8.96 (d, J = 6.6 Hz, 2H), 8.53 (d, J = 6.3 Hz, 2H), 8.02 (s, 1H), 5.76 (s, 1H), 4.30-4.17 (m, 4H), 3.92-3.86 (m, 4H), 2.02-1.93 (m, 1H), 1 10-1.04 (m, 2H), 0.81-0.76 (m, 2H). |
| 70 | N-(5-cyclobutyl-1H- pyrazol-3-yl)-4- morpholino-6- (pyridin-4- yl)furo[3,2- d]pyrimidin-2-amine, hydrogen chloride | 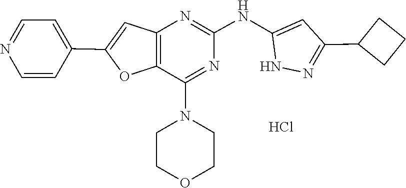 [SMILES: c1cc(-c2cc3nc(Nc4cc(C5CCC5)n[nH]4)nc(N4CCOCC4)c3o2)ccn1 ; Cl] | 20 | LC-MS (ESI+): m/z 418 (MH⁺). ¹HNMR (300 MHz, CD₃OD) δ 8.99 (d, J = 6.6 Hz, 2H), 8.60 (d, J = 6.9 Hz, 2H), 8.07 (s, 1H), 5.99 (s, 1H), 4.37-4.24 (m, 4H), 3.96-3.91 (m, 4H), 3.68-3.53 (m, 1H), 2.47-2.41 (m, 2H), 2.37-2.03 (m, 3H), 2.00-1.97 (m, 1H). |
| 71 | 2-((3- (cyclobutylmethyl)- 1H-pyrazol-5- yl)methyl)-4- morpholino-6- (pyridin-4- yl)furo[3,2- d]pyrimidine, hydrogen chloride | 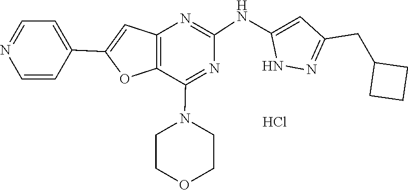 [SMILES: c1cc(-c2cc3nc(Nc4cc(CC5CCC5)n[nH]4)nc(N4CCOCC4)c3o2)ccn1 ; Cl] | 20 | LC-MS (ESI+): m/z 432 (MH⁺). ¹HNMR (300 MHz, CD₃OD) δ 8.96 (d, J = 6.6 Hz, 2H), 8.52 (d, J = 6.3 Hz, 2H), 8.02 (s, 1H), 5.89 (s, 1H), 4.35-4.27 (m, 4H), 3.94-3.89 (m, 4H), 2.79-2.76 (m, 2H), 2.70-2.60 (m, 1H), 2.14-2.12 (m, 2H), 1.97-1.83 (m, |
| | | | | 2H), 1,80-1.74 (m, 2H). |
| 72 | 4-morpholino-6- (pyridin-4-yl)-N-(5- (trifluoromethyl)-1H- pyrazol-3- yl)furo[3,2- d]pyrimidin-2-amine, hydrogen chloride | 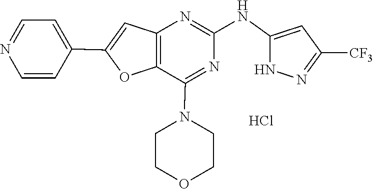 [SMILES: FC(F)(F)c1cc(Nc2nc(N3CCOCC3)c3oc(-c4ccncc4)cc3n2)[nH]n1 ; Cl] | 20 | LC-MS (ESI+): m/z 432 (MH⁺). ¹HNMR (300 MHz, CD₃OD) δ 8.98 (d, J = 6.3 Hz, 2H), 8.58 (d, J = 6.3 Hz, 2H), 8.02 (s, 1H), 6.57 (s, 1H), 4.35-4.20 (m, 4H), 3.96-3.92 (m, 4H). |
| 73 | N-(5-benzyl-1H- pyrazol-3-yl)-4- morpholino-6- (pyridin-4- yl)furo[3,2- d]pyrimidin-2-amine, hydrogen chloride | 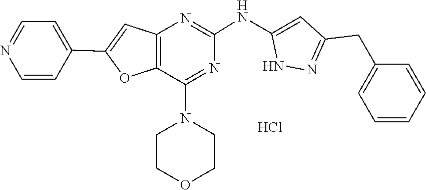 [SMILES: c1ccc(Cc2cc(Nc3nc(N4CCOCC4)c4oc(-c5ccncc5)cc4n3)[nH]n2)cc1 ; Cl] | 20 | LC-MS (ESI+): m/z 454 (MH⁺). ¹HNMR (300 MHz, CD₃OD) δ 8.91 (brs, 2H), 8.58 (brs, 2H), 8.02 (s, 1H), 7.43-7.24 (m, 5H), 5.89 (s, 1H), 4.40-4.17 (m, 4H), 4.05 (s, 2H), 3.92-3.84 (m, 4H). |
| 74 | N-(5-cyclopentyl-1H- pyrazol-3-yl)-4- morpholino-6- (pyridin-4- yl)furo[3,2- d]pyrimidin-2-amine, hydrogen chloride | 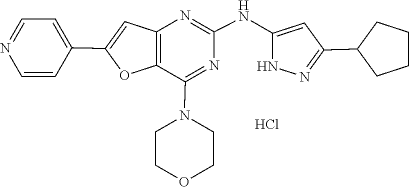 [SMILES: c1cc(-c2cc3nc(Nc4cc(C5CCCC5)n[nH]4)nc(N4CCOCC4)c3o2)ccn1 ; Cl] | 20 | LC-MS (ESI+): m/z 432 (MH⁺). ¹HNMR (300 MHz, DMSO-d₆) δ 11.02 (s, 1H), 8.86 (d, J = 5.7 Hz, 2H), 8.16 (d, J = 4.8 Hz, 2H), 7.90 (s, 1H), 5.98 (s, 1H), 4.18-4.05 (m, 4H), 3.90-3.79 (m, 4H), 3.12-3.08 (m, 1H), 2.10-1.90 (m, 2H), 1.72-1.61 (m, 6H). |
| 75 | 4-morpholino-6- (pyridin-4-yl)-N-(5- (thiophen-2-yl)-1H- pyrazol-3- yl)furo[3,2- d]pyrimidin-2-amine, hydrogen chloride | 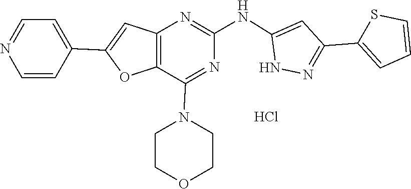 [SMILES: c1csc(-c2cc(Nc3nc(N4CCOCC4)c4oc(-c5ccncc5)cc4n3)[nH]n2)c1 ; Cl] | 20 | LC-MS (ESI+): m/z 446 (MH⁺). ¹HNMR (300 MHz, DMSO-d₆) δ 8.92 (d, J = 6.0 Hz, 2H), 8.29 (d, J = 5.1 Hz, 2H), 7.96 (s, 1H), 7.62 (d, J = 5.1 Hz, 1H), 7.55 (d, J = 3.3 Hz, 1H), 7.17(dd, 7 = 7.8, 3.6 Hz, 1H), 6. 46 (s, 1H), 4.28-4.16 (m, 4H), 3.94-3.86 (m, 4H). |
| 76 | 4-morpholino-6- (pyridin-4-yl)-N-(5- (thiophen-3-yl)- 1H- pyrazol-3- yl)furo[3,2- d]pyrimidin-2-amine, hydrogen chloride | 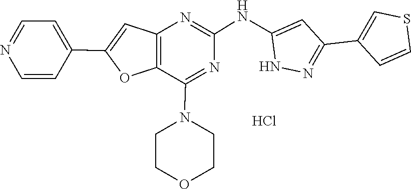 [SMILES: c1cc(-c2cc3nc(Nc4cc(-c5ccsc5)n[nH]4)nc(N4CCOCC4)c3o2)ccn1 ; Cl] | 20 | LC-MS (ESI+): m/z 446 (MH⁺). ¹HNMR (300 MHz, CD₃OD) δ 8.97 (d, J = 6.9 Hz, 2H), 8.67 (d, J = 6.6 Hz, 2H), 8.08 (s, 1H), 7.81 (dd, J = 2.7, 1.2 Hz, 1H), 7.57 (dd, J = 5.1, 2.4 Hz, 1H), 7.45 (dd, J = 4.8, 1.2 Hz, 1H), 6. 35 (s, 1H), 4.40-4.11 (m, 4H), |
| | | | | 3.98-3.91 (m, 4H). |
| 77 | N-(5-isopropyl- 1H- pyrazol-3-yl)-4- morpholino-6- (pyridin-4- yl)furo[3,2- d]pyrimidin-2-amine, hydrogen chloride | 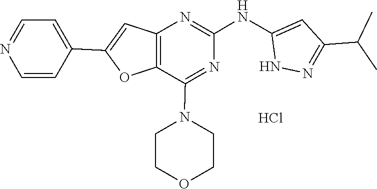 [SMILES: CC(C)c1cc(Nc2nc(N3CCOCC3)c3oc(-c4ccncc4)cc3n2)[nH]n1 ; Cl] | 20 | LC-MS (ESI+): m/z 406 (MH⁺). ¹HNMR (300 MHz, CD₃OD) δ 8.95 (d, J = 6.6 Hz, 2H), 8.52 (d, J = 6.9 Hz, 2H), 8.03 (s, 1H), 5.92 (s, 1H), 4.36-4.23 (m, 4H), 3.96-3.92 (m, 4H), 3.10-3.03 (m, 1H), 1.32 (d, J = 6.9 Hz, 6H). |
| 79 | 2-((5- (cyclopropylmethyl)- 1H-pyrazol-3- yl)amino)-N-methyl- 4-morpholinofuro[3,2- d]pyrimidine-6- carboxamide, hydrogen chloride | 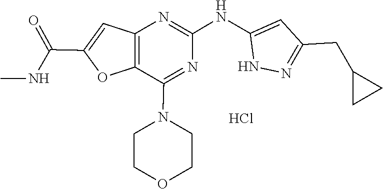 [SMILES: CNC(=O)c1cc2nc(Nc3cc(CC4CC4)n[nH]3)nc(N3CCOCC3)c2o1 ; Cl] | 14 (1.1) and 21 | LC-MS (ESI+): m/z 398 (MH⁺). ¹HNMR (300 MHz, CD₃OD) δ 7.51 (s, 1H), 5.97 (s, 1H), 4.25-4.151 (m, 4H), 3.92-3.86 (m, 4H), 2.96 (s, 3H), 2.59 (d, J = 6.9 Hz, 2H), 1.07-1.02 (m, 1H), 0.61-0.56 (m, 2H), 0.25-0.21 (m, 2H). |
| 80 | 2-((5-cyclobutyl-1H- pyrazol-3-yl)amino)- N-methyl-4- morpholinofuro[3,2- d]pyrimidine-6- carboxamide, hydrogen chloride | 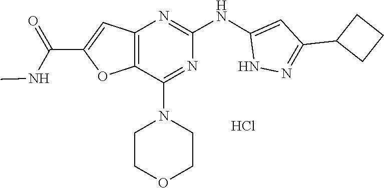 [SMILES: CNC(=O)c1cc2nc(Nc3cc(C4CCC4)n[nH]3)nc(N3CCOCC3)c2o1 ; Cl] | 14 (1.1) and 21 | LC-MS (ESI+): m/z 398 (MH⁺). ¹HNMR (300 MHz, CD₃OD) δ 7.51 (s, 1H), 5.95 (s, 1H), 4.30-4.11 (m, 4H), 3.92-3.86 (m, 4H), 3.65-3.54 (m, 1H), 2.96 (s, 3H), 2.45-2.37 (m, 2H), 2.28-2.05 (m, 3H), 2.02-1.97 (m, 1H). |
| 81 | N-methyl-4- morpholino-2-((5- (trifluoromethyl)-1H- pyrazol-3- yl)amino)furo[3,2- d]pyrimidine-6- carboxamide, hydrogen chloride | 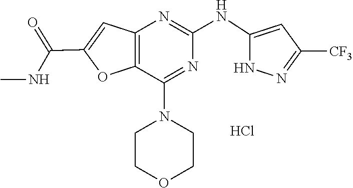 [SMILES: CNC(=O)c1cc2nc(Nc3cc(C(F)(F)F)n[nH]3)nc(N3CCOCC3)c2o1 ; Cl] | 14 (1.1) and 21 | LC-MS (ESI+): m/z 412 (MH⁺). ¹HNMR (300 MHz, CD₃OD) δ 7.44 (s, 1H), 6.43 (s, 1H), 4.23-4.11 (m, 4H), 3.91-3.86 (m, 4H), 3.07 (s, 3H). |
| 82 | 4-morpholino-N-[5- (4-pyridyl)-1H- pyrazol-3- yl]furo[3,2- d]pyrimidin-2-amine, hydrogen chloride | 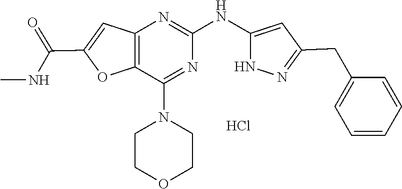 [SMILES: CNC(=O)c1cc2nc(Nc3cc(Cc4ccccc4)n[nH]3)nc(N3CCOCC3)c2o1 ; Cl] | 14 (1.1) and 21 | LC-MS (ESI+): m/z 434 (MH⁺). ¹HNMR (300 MHz, CD₃OD) δ 7.51 (s, 1H), 7.35-7.21 (m, 5H), 5.86 (s, 1H), 4.30-4.11 (m, 4H), 4.03 (s, 2H), 3.88-3.81 (m, 4H), 2.93 (s, 3H). |
| 83 | 2-((5-cyclopentyl- 1H-pyrazol-3- yl)amino)-N-methyl- 4- morpholinofuro[3,2- d]pyrimidine-6- carboxamide, hydrogen chloride | 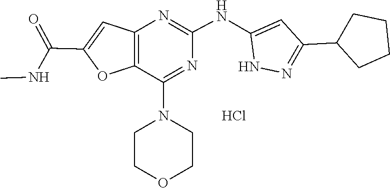 [SMILES: CNC(=O)c1cc2nc(Nc3cc(C4CCCC4)n[nH]3)nc(N3CCOCC3)c2o1 ; Cl] | 14 (1.1) and 21 | LC-MS (ESI+): m/z 412 (MH⁺). ¹HNMR (300 MHz, CD₃OD) δ 7.52 (s, 1H), 5.89 (s, 1H), 4.26-4.15 (m, 4H), 3.88-3.81 (m, 4H), 3.16-3.05 (m, 1H), 2.96 (s, 3H), 2.14-2.08 (m, 2H), 1.82-1.66 (m, 6H). |
| 84 | 2-((5- (cyclopentylmethyl)- 1H-pyrazol-3- yl)amino)-N-methyl- 4- morpholinofuro[3,2- d]pyrimidine-6- carboxamide, hydrogen chloride | 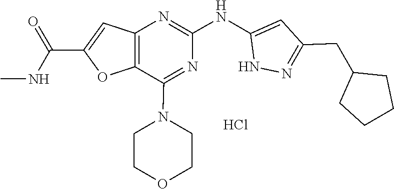 [SMILES: CNC(=O)c1cc2nc(Nc3cc(CC4CCCC4)n[nH]3)nc(N3CCOCC3)c2o1 ; Cl] | 14 (1.1) and 21 | LC-MS (ESI+): m/z 426 (MH⁺). ¹HNMR (300 MHz, DMSO-d₆) δ 7.52 (s, 1H), 5.89 (s, 1H), 4.28-4.15 (m, 4H), 3.90-3.80 (m, 4H), 2.96 (s, 3H), 2.67 (d, J = 7.5 Hz, 2H), 2.23-2.12 (m, 1H), 1.81-1.76 (m, 2H), 1.68-1.60 (m, 4H), 1.30-1.20 (m, 2H). |
| 85 | N-methyl-4- morpholino-2-((5- (thiophen-2-yl)-1H- pyrazol-3- yl)amino)furo[3,2- d]pyrimidine-6- carboxamide, hydrogen chloride | 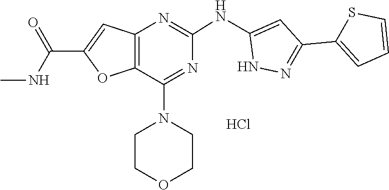 [SMILES: CNC(=O)c1cc2nc(Nc3cc(-c4cccs4)n[nH]3)nc(N3CCOCC3)c2o1 ; Cl] | 14 (1.1) and 21 | LC-MS (ESI+): m/z 426 (MH⁺). ¹HNMR (300 MHz, DMSO-d₆) δ 11.06 (s, 1H), 9.03 (d, J = 6.6 Hz, 1H), 7.64 (d, J = 4.5 Hz, 1H), 7.57 (d, J = 3.3 Hz, 1H), 7.52 (s, 1H), 7.17 (dd, J = 7.8, 3.6 Hz, 1H), 6.42 (s, 1H), 4.20-4.05 (m, 4H), 3.90-3.81 (m, 4H), |
| | | | | 2.84 (d, J = 4.5 Hz, 3H). |
| 86 | N-methyl-4- morpholino-2-((5- (thiophen-3-yl)-1H- pyrazol-3- yl)amino)furo[3,2- d]pyrimidine-6- carboxamide, hydrogen chloride | 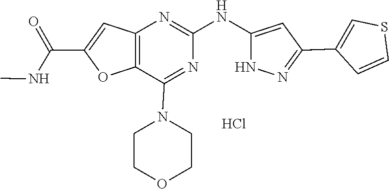 [SMILES: CNC(=O)c1cc2nc(Nc3cc(-c4ccsc4)n[nH]3)nc(N3CCOCC3)c2o1 ; Cl] | 14 (1.1) and 21 | LC-MS (ESI+): m/z 426 (MH⁺). ¹HNMR (300 MHz, CD₃OD) δ 7.79 (s, 1H), 7.63-7.55 (m, 1H), 7.51 (s, 1H), 7.45 (d, J = 4.8 Hz, 1H), 6.34 (s, 1H), 4.30-4.18 (m, 4H), 3.92-3.82 (m, 4H), 2.97 (s, 3H). |
| 87 | 2-((5-ethyl-1H- pyrazol-3-yl)amino)- N-methyl-4- morpholinofuro[3,2- d]pyrimidine-6- carboxamide, hydrogen chloride | 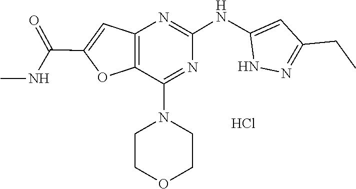 [SMILES: CCc1cc(Nc2nc(N3CCOCC3)c3oc(C(=O)NC)cc3n2)[nH]n1 ; Cl] | 14 (1.1) and 21 | LC-MS (ESI+): m/z 372 (MH⁺). ¹HNMR (300 MHz, CD₃OD) δ 7.51 (s, 1H), 5.90 (s, 1H), 4.28-4.14 (m, 4H), 3.92-3.86 (m, 4H), 2.96 (s, 3H), 2.75 (q, J = 7.5 Hz, 2H), 1.29 (t, J = 7.8 Hz, 3H). |
| 88 | 2-((5-isopropyl-1H- pyrazol-3-yl)amino)- N-methyl-4- morpholinofuro[3,2- d]pyrimidine-6- carboxamide, hydrogen chloride | 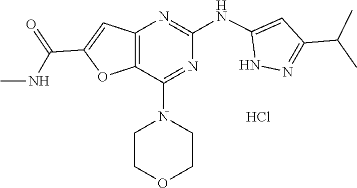 [SMILES: CNC(=O)c1cc2nc(Nc3cc(C(C)C)n[nH]3)nc(N3CCOCC3)c2o1 ; Cl] | 14 (1.1) and 21 | LC-MS (ESI+): m/z 386 (MH⁺). ¹HNMR (300 MHz, CD₃OD) δ 7.52 (s, 1H), 5.90 (s, 1H), 4.28-4.15 (m, 4H), 3.91-3.85 (m, 4H), 3.08-2.99 (m, 1H), 2.96 (s, 3H), 1.31 (d, J = 6.9 Hz, 6H). |

### Example 22: Compound 89 Using General Synthetic Route 22

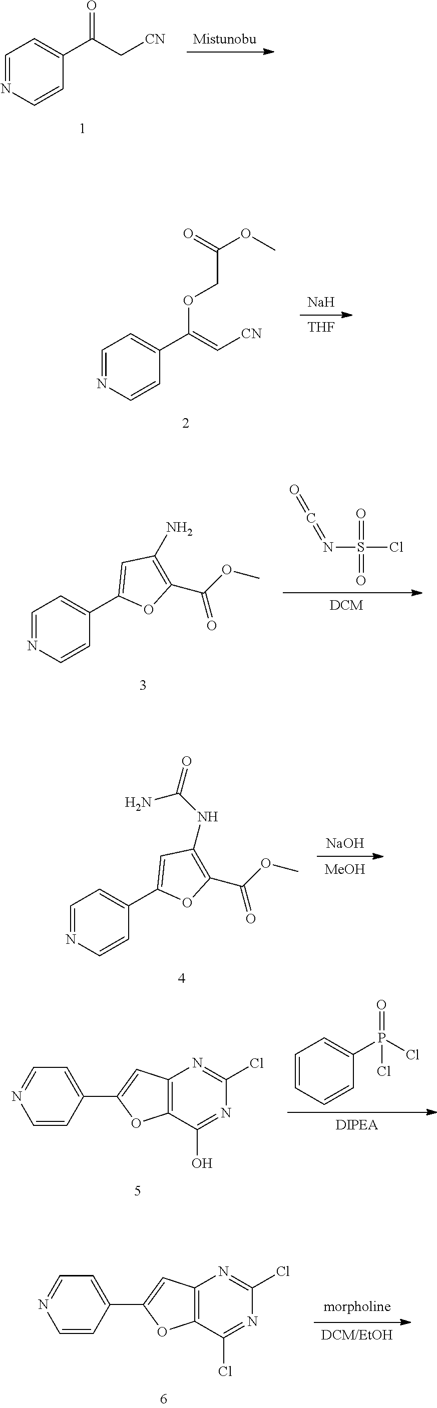

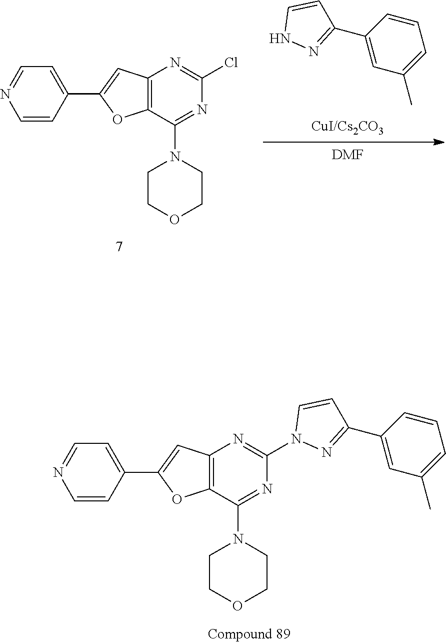

### 1.1) Synthesis of (Z)-methyl 2-((2-cyano-1-(pyridin-4-yl)vinyl)oxy)acetate

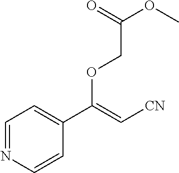 [SMILES: COC(=O)CO/C(=C\C#N)c1ccncc1]

To a solution of triphenylphosphine (3.5 g, 13.4 mmol) in dry tetrahydrofuran (THF) was added Diethyl azodicarboxylate (DEAD) (2.3 g, 13.4 mmol), 3-oxo-3-(pyridin-4-yl)propanenitrile (1.5 g, 10.3 mmol) and methyl 2-hydroxyacetate (1.2 g, 13.4 mmol) under N₂. The reaction was stirred at room temperature overnight. Upon the completion of the reaction as monitored by thin layer chromatography (TLC), the reaction mixture was concentrated directly and purified by silica gel column chromatography with a gradient elution of 1% MeOH/DCM to 2% MeOH/DCM to provide 4.4 g of impure (Z)-methyl2-((2-cyano-1-(pyridin-4-yl)vinyl)oxy)acetate containing triphenylphosphine oxide as a yellow solid. LC-MS (ESI+): m/z 219 (MH⁺).

### 1.2) Synthesis of methyl 3-amino-5-(pyridin-4-yl)furan-2-carboxylate

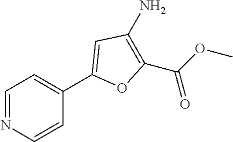 [SMILES: COC(=O)c1oc(-c2ccncc2)cc1N]

To a solution of impure (Z)-methyl2-((2-cyano-1-(pyridin-4-yl)vinyl)oxy)acetate (4.6 g, 21.1 mmol) in dry THE at 0° C. was added NaH (1.5 g, 31.6 mmol). The reaction was warmed to room temperature and stirred at room temperature for 2 h. Upon the completion of the reaction as monitored by TLC, the reaction was quenched with a saturated NH₄C₁ solution and the pH was adjusted to 3 using 2 N HCl aqueous solutions. The aqueous solution was washed with ethyl acetate (3×50 mL) to remove some impurities. To the aqueous solution was added a saturated Na₂CO₃ solution to adjust the pH to 11 and extracted with DCM/MeOH (10/1, 3×60 mL). The combined organic phase was dried over anhydrous Na₂SO₄, filtrated and concentrated to provide 1.35 g of crude methyl 3-amino-5-(pyridin-4-yl)furan-2-carboxylate as an oil. The crude product was used directly for the next step without further purification. LC-MS (ESI+): m/z 219 (MH⁺). ¹HNMR (300 MHz, CDCl₃) δ 8.66 (dd, J=4.5, 1.5 Hz, 2H), 7.58 (dd, J=4.8, 1.2 Hz, 2H), 6.58 (s, 1H), 4.67 (brs, 2H), 3.93 (s, 3H).

### 1.3) Synthesis of methyl 5-(pyridin-4-yl)-3-ureidofuran-2-carboxylate

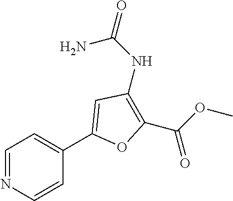 [SMILES: COC(=O)c1oc(-c2ccncc2)cc1NC(N)=O]

To a solution of methyl 3-amino-5-(pyridin-4-yl)furan-2-carboxylate (1.94 g, 8.9 mmol) in DCM (40 mL) at −78° C. under N₂ was added sulfurisocyanatidic chloride (3.79 g, 26.7 mmol) dropwise. After addition, the reaction was warmed to room temperature and stirred at room temperature for 1 h. The organic solvent was removed by evaporation in vacuo. To the resulting residue was added 6 N HCl (10 mL) aqueous solutions. The mixture was heated to reflux for 30 min. Upon the completion of the reaction as monitored by TLC, the reaction was cooled to room temperature and the pH was adjusted to 9 using a saturated NaHCO₃ solution. A large amount of solid was precipitated. After filtration, the filter cake was washed with water and dried to provide 2.7 g of crude methyl5-(pyridin-4-yl)-3-ureidofuran-2-carboxylate as a yellow solid. LC-MS (ESI+): m/z 262 (MH⁺). ¹HNMR (300 MHz, CD₃OD) δ 8.61 (dd, J=4.8, 1.5 Hz, 2H), 7.89 (s, 1H), 7.78 (dd, J=5.1, 1.5 Hz, 2H), 3.95 (s, 3H).

### 1.4) Synthesis of 6-(pyridin-4-yl)furo[3,2-d]pyrimidine-2,4-diol

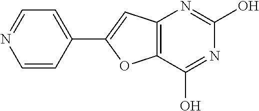 [SMILES: Oc1nc(O)c2oc(-c3ccncc3)cc2n1]

To a solution of crude methyl 5-(pyridin-4-yl)-3-ureidofuran-2-carboxylate (2.7 g, 10.3 mmol) in MeOH (40 mL) was added 1.5 N NaOH (15 mL). The reaction was heated to reflux for 1.5 h. Upon the completion of the reaction as monitored by TLC, the solvent MeOH was removed by evaporation in vacuo. To the resulting residue were added 6 N HCl solutions until the pH to 2. A large amount of solid was precipitated. After filtration, the filter cake was washed with water and dried to provide crude 2.1 g of 6-(pyridin-4-yl)furo[3,2-d]pyrimidine-2,4-diol as a yellow solid. LC-MS (ESI+): m/z 230 (MH⁺). ¹HNMR (300 MHz, DMSO-d₆) δ 11.61 (s, 1H), 11.37 (s, 1H), 8.89 (d, J=6.6 Hz, 2H), 8.24 (d, J=6.3 Hz, 2H), 7.63 (s, 1H).

### 1.5) Synthesis of 2,4-dichloro-6-(pyridin-4-yl)furo[3,2-d]pyrimidine

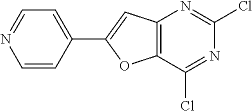 [SMILES: Clc1nc(Cl)c2oc(-c3ccncc3)cc2n1]

To a solution of 6-(pyridin-4-yl)furo[3,2-d]pyrimidine-2,4-diol (1.5 g, 6.54 mmol) in phenylphosphonic dichloride (30 mL) was added DIPEA (8.43 g, 65.4 mmol). The reaction mixture was heated to 120° C. overnight. Upon the completion of the reaction as monitored by TLC, the reaction was cooled to room temperature, a saturated NaHCO₃ solution was added to adjust the pH to 8. The aqueous solution was extracted with EtOAc (3×50 mL). The combined organic phase was dried over anhydrous Na₂SO₄, filtrated and concentrated. The resulting residue was purified by silica gel column chromatography with a gradient elution of 50% EtOAc/PE to 75% EtOAc/PE to provide 2,4-dichloro-6-(pyridin-4-yl) furo[3,2-d]pyrimidine (1.6 g, 6.9 mmol) as a yellow solid. LC-MS (ESI+): m/z 266/268 (MH⁺). ¹HNMR (300 MHz, CDCl₃) δ 8.85 (dd, J=4.8, 1.5 Hz, 2H), 7.82 (dd, J=4.5, 1.5 Hz, 2H), 7.38 (s, 1H).

### 1.6) Synthesis of 2-chloro-4-morpholino-6-(pyridin-4-yl)furo[3,2-d]pyrimidine

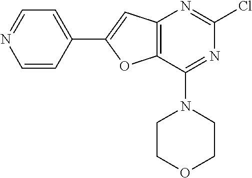 [SMILES: Clc1nc(N2CCOCC2)c2oc(-c3ccncc3)cc2n1]

To a solution of 2,4-dichloro-6-(pyridin-4-yl)furo[3,2-d]pyrimidine (1.6 g, 6.9 mmol) in DCM/EtOH (1/3, 120 mL) was added morpholine (0.91 g, 10.5 mmol) and K₂CO₃ (1.91 g, 14 mmol). The reaction was stirred at room temperature for 2 h. Upon the completion of the reaction as monitored by TLC, the reaction mixture was concentrated directly and purified by silica gel column chromatography with a gradient elution of 2% MeOH/DCM to 3% MeOH/DCM to provide 2-chloro-4-morpholino-6-(pyridin-4-yl)furo[3,2-d]pyrimidine (770 mg, 2.43 mmol) as a yellow solid. LC-MS (ESI+): m/z 317/319 (MH⁺). ¹HNMR (300 MHz, DMSO-d₆) δ 8.76 (d, J=6.0 Hz, 2H), 7.97 (d, J=6.0 Hz, 2H), 7.82 (s, 1H), 4.06-3.97 (m, 4H), 3.82-3.75 (m, 4H).

### 1.7) Synthesis of 4-morpholino-6-(pyridin-4-yl)-2-(3-(m-tolyl)-1H-pyrazol-1-yl)furo [3,2-d]pyrimidine

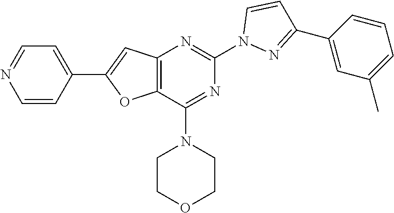 [SMILES: Cc1cccc(-c2ccn(-c3nc(N4CCOCC4)c4oc(-c5ccncc5)cc4n3)n2)c1]

To a solution of 2-chloro-4-morpholino-6-(pyridin-4-yl)furo[3,2-d]pyrimidine (100 mg, 0.32 mmol) in DMF (10 mL) was added 3-(m-tolyl)-1H-pyrazole (55 mg, 0.35 mmol), Cs₂CO₃ (210 mg, 0.64 mmol) and CuI (12 mg, 0.064 mmol). The reaction mixture was stirred at 110° C. overnight. Upon the completion of the reaction as monitored by TLC, the reaction was cooled to room temperature, quenched with water (10 mL) and extracted with EtOAc (3×10 mL). The combined organic phase was dried over anhydrous Na₂SO₄, filtrated and concentrated under reduced pressure. The resulting residue was purified by silica gel column chromatography with a gradient elution of 1% MeOH/DCM to 4% MeOH/DCM to provide 4-morpholino-6-(pyridin-4-yl)-2-(3-(m-tolyl)-1H-pyrazol-1-yl)furo [3,2-d]pyrimidine (28 mg, 0.064 mmol) as a white solid. LC-MS (ESI+): m/z 439 (MH⁺). ¹HNMR (300 MHz, DMSO-d₆) δ 8.78 (brs, 2H), 8.72 (s, 1H), 8.05-8.01 (m, 2H), 7.91 (s, 1H), 7.81 (s, 1H), 7.55 (d, J=7.5 Hz, 1H), 7.36 (t, J=7.5 Hz, 1H), 7.20 (d, J=7.2 Hz, 1H), 7.03 (s, 1H), 4.18-4.12 (m, 4H), 3.90-3.84 (m, 4H), 2.40 (s, 3H).

### Example 23: Compound 90 Using General Synthetic Route 23

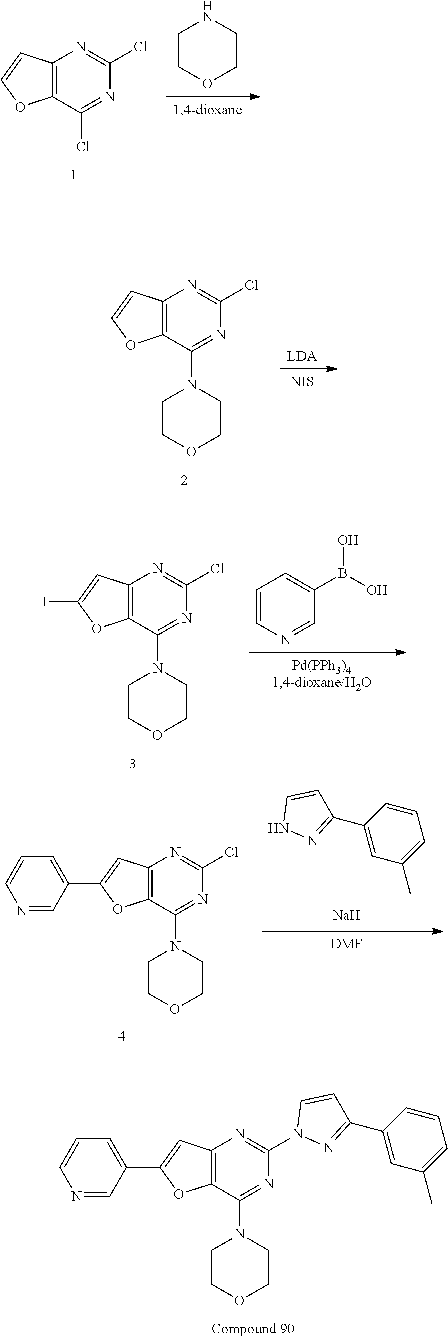

### 1.1) Synthesis of 2-chloro-4-morpholinofuro[3, 2-d]pyrimidine

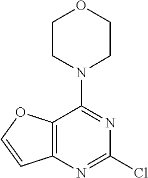 [SMILES: Clc1nc(N2CCOCC2)c2occc2n1]

To a solution of 2,4-dichlorofuro[3,2-d]pyrimidine (6.46 g, 34.2 mmol mmol) in 1,4-dioxane (100 mL) was added morpholine (5.95 g, 68.4 mmol). The reaction was stirred at room temperature for 30 min. Upon the completion of the reaction as monitored by TLC, the reaction mixture was concentrated directly and the resulting residue was purified by silica gel column chromatography with a gradient elution of 10% EtOAc/PE to 20% EtOAc/PE to provide 2-chloro-4-morpholinofuro[3,2-d]pyrimidine (7.5 g, 40.1 mmol) as a white solid. LC-MS (ESI+): m/z 240/242 (MH⁺). HNMR (300 MHz, CDCl₃) δ 7.74 (d, J=1.8 Hz, 1H), 6.79 (d, J=2.1 Hz, 1H), 4.05-4.02 (m, 4H), 3.85-3.82 (m, 4H).

### 1.2) Synthesis of 2-chloro-6-iodo-4-morpholinofuro[3,2-d]pyrimidine

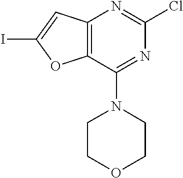 [SMILES: Clc1nc(N2CCOCC2)c2oc(I)cc2n1]

To a solution of 2-chloro-4-morpholinofuro[3,2-d]pyrimidine (2.0 g, 0.83 mmol) in anhydrous THF (30 mL) at −78° C. under N₂ was added LDA (1.33 mL, 2M, 2.66 mmol). After stirred at −78° C. for 1 h, to the solution was added a solution of NIS (2.25 g, 1.0 mmol) in anhydrous THF (10 mL). Upon the completion of the reaction as monitored by TLC, the reaction was quenched with water (50 mL) and extracted with DCM (3×50 mL). The combined organic phase was dried over anhydrous Na₂SO₄, filtrated and concentrated under reduced pressure. The resulting residue was purified by silica gel column chromatography with a gradient elution of 5% EtOAc/PE to 10% EtOAc/PE to provide 2-chloro-6-iodo-4-morpholinofuro[3,2-d]pyrimidine (1.6 g, 4.4 mmol) as yellow solid. LC-MS (ESI+): m/z 366/368 (MH⁺). ¹HNMR (300 MHz, CDCl₃) δ 6.97 (s, 1H), 4.01-3.98 (m, 4H), 3.85-3.82 (m, 4H).

### 1.3) Synthesis of 2-chloro-4-morpholino-6-(pyridin-3-yl)furo[3,2-d]pyrimidine

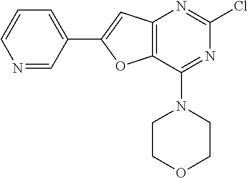 [SMILES: Clc1nc(N2CCOCC2)c2oc(-c3cccnc3)cc2n1]

To a solution of 2-chloro-6-iodo-4-morpholinofuro[3,2-d]pyrimidine (500 mg, 1.37 mmol) in 1,4-dioxane/H₂O (2/1, 30 mL) was added pyridin-3-ylboronic acid (168 mg, 1.37 mmol), K₂CO₃ (567 mg, 4.1 mmol) and Pd(PPh₃)₄ (158 mg, 0.13 mmol). The reaction was stirred at 90° C. for 5 h. Upon the completion of the reaction as monitored by TLC, the reaction solution was concentrated directly and the resulting residue was purified by silica gel column chromatography with a gradient elution of 1% MeOH/DCM to 3% MeOH/DCM to provide 2-chloro-4-morpholino-6-(pyridin-3-yl)furo[3,2-d] pyrimidine (410 mg, 1.29 mmol) as a light yellow solid. LC-MS (ESI+): m/z 317/319 (MH⁺). ¹HNMR (300 MHz, CDCl₃) δ 9.07 (d, J=1.5 Hz, 1H), 8.69-8.68 (m, 1H), 8.06 (d, J=8.1 Hz, 1H), 7.46-7.42 (m, 1H), 7.08 (s, 1H), 4.11-4.08 (m, 4H), 3.90-3.87 (m, 4H).

### 1.4) Synthesis of 4-morpholino-6-(pyridin-3-yl)-2-(3-(m-tolyl)-1H-pyrazol-1-yl) furo[3, 2-d]pyrimidine

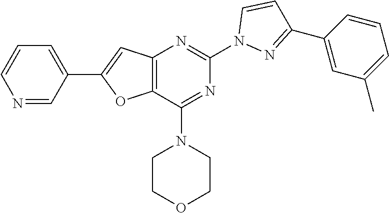 [SMILES: Cc1cccc(-c2ccn(-c3nc(N4CCOCC4)c4oc(-c5cccnc5)cc4n3)n2)c1]

To a solution of 2-chloro-4-morpholino-6-(pyridin-3-yl)furo[3,2-d]pyrimidine (100 mg, 0.32 mmol) in DMF (10 mL) was added 3-(m-tolyl)-1H-pyrazole (60 mg, 0.38 mmol) and NaH (25 mg, 0.63 mmol). The reaction was stirred at 90° C. overnight. Upon the completion of the reaction as monitored by TLC, the reaction was quenched with water (50 mL) and extracted with EtOAc (3×30 mL). The combined organic phase was dried over anhydrous Na₂SO₄, filtrated and concentrated under reduced pressure. The resulting residue was purified by silica gel column chromatography with a gradient elution of 1% MeOH/DCM to 3% MeOH/DCM to provide 4-morpholino-6-(pyridin-3-yl)-2-(3-(m-tolyl)-1H-pyrazol-1-yl)furo[3,2-d]pyrimidine (80 mg, 0.18 mmol) as an off-white solid. LC-MS (ESI+): m/z 439 (MH⁺). ¹HNMR (300 MHz, DMSO-d₆) δ 9.30 (s, 1H), 8.71-8.69 (m, 2H), 8.43 (d, J=7.5 Hz, 1H), 7.81-7.74 (m, 3H), 7.63-7.60 (m, 1H), 7.35 (t, J=7.5 Hz, 1H), 7.20 (d, J=7.8 Hz, 1H), 7.02 (d, J=2.7 Hz, 1H), 4.15-4.11 (m, 4H), 3.90-3.82 (m, 4H), 2.41 (s, 3H).

### Example 24: Compound 91 Using General Synthetic Route 24

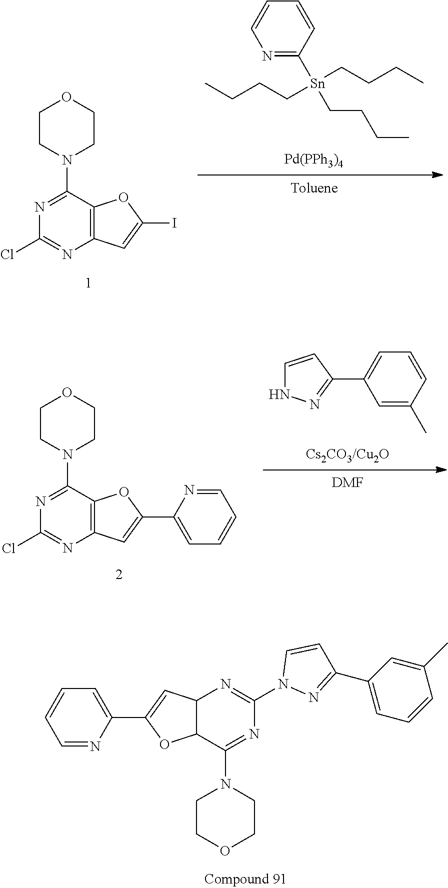

### 1.1) Synthesis of 2-chloro-4-morpholino-6-(pyridin-2-yl)furo[3,2-d]pyrimidine

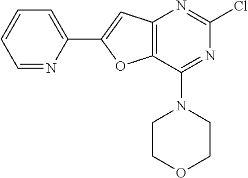 [SMILES: Clc1nc(N2CCOCC2)c2oc(-c3ccccn3)cc2n1]

A solution of 2-chloro-6-iodo-4-morpholinofuro[3,2-d]pyrimidine (1 g, 2.7 mmol), 2-(tributylstannyl)pyridine (1.2 g, 3.3 mmol) and Pd(PPh₃)₄ (155 mg, 0.14 mmol) in toluene (5 mL) was heated to 90° C. overnight. Upon the completion of the reaction as monitored by TLC, the reaction was cooled to room temperature, diluted with water and extracted with DCM/MeOH (15/1, 3×50 mL). The combined organic phase was dried over anhydrous Na₂SO₄, filtrated and concentrated under reduced pressure. The resulting residue was purified by silica gel column chromatography with a gradient elution of 1% MeOH/DCM to 5% MeOH/DCM to provide 2-chloro-4-morpholino-6-(pyridin-2-yl)furo[3,2-d] pyrimidine (352 mg, 1.11 mmol) as a yellow solid. LC-MS (ESI+): m/z 317/319 (MH⁺). ¹HNMR (300 MHz, DMSO-d₆) δ 8.74-8.70 (m, 1H), 8.10 (d, J=8.1 Hz, 1H), 8.00 (d, J=7.5 Hz, 1H), 7.63-7.49 (m, 2H), 4.05-3.97 (m, 4H), 3.82-3.76 (m, 4H).

### 1.2) Synthesis of 4-morpholino-6-(pyridin-2-yl)-2-(3-(m-tolyl)-1H-pyrazol-1-yl) furo[3,2-d]pyrimidine

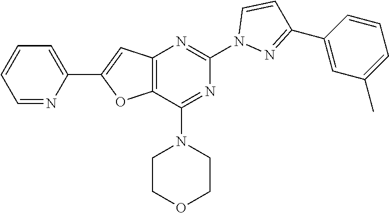 [SMILES: Cc1cccc(-c2ccn(-c3nc(N4CCOCC4)c4oc(-c5ccccn5)cc4n3)n2)c1]

To a solution of 2-chloro-4-morpholino-6-(pyridin-2-yl)furo[3,2-d]pyrimidine (80 mg, 0.25 mmol) in DMF (10 mL) was added 3-(m-tolyl)-1H-pyrazole (48 mg, 0.30 mmol), Cs₂CO₃ (165 mg, 0.51 mmol) and Cu₂O (3.6 mg, 0.025 mmol). The reaction was stirred at 110° C. overnight. Upon the completion of the reaction as monitored by TLC, the reaction was cooled to room temperature, quenched with water (10 mL) and extracted with DCM/MeOH (15/1, 3×20 mL). The combined organic phase was dried over anhydrous Na₂SO₄, filtrated and concentrated under reduced pressure. The resulting residue was purified by preparative TLC to provide 4-morpholino-6-(pyridin-2-yl)-2-(3-(m-tolyl)-1H-pyrazol-1-yl)furo[3,2-d]pyrimidine (39 mg, 0.089 mmol) as a white solid. LC-MS (ESI+): m/z 439 (MH⁺). ¹HNMR (300 MHz, DMSO-d₆) δ 8.76-8.71 (m, 2H), 8.12 (d, J=7.5 Hz, 1H), 8.01 (t, J=7.5 Hz, 1H), 7.80 (s, 1H), 7.75 (d, J=7.2 Hz, 1H), 7.68 (s, 1H), 7.62-7.49 (m, 1H), 7.36 (t, J=7.5 Hz, 1H), 7.20 (d, J=7.2 Hz, 1H), 7.02 (s, 1H), 4.18-4.12 (m, 4H), 3.90-3.84 (m, 4H), 2.40 (s, 3H).

### Example 25: Compound 92 Using General Synthetic Route 25

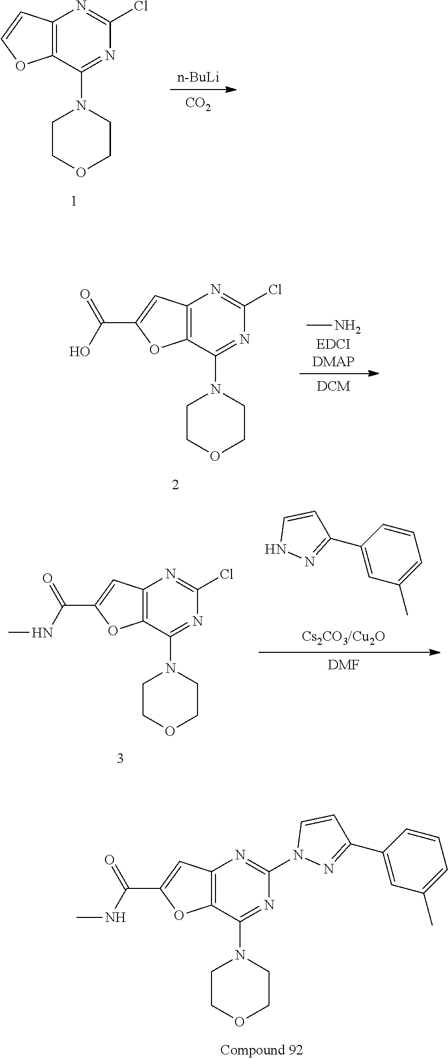

### 1.1) Synthesis of 2-chloro-4-morpholinofuro[3, 2-d]pyrimidine-6-carboxylic acid

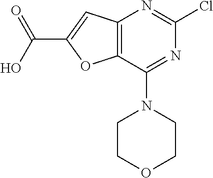 [SMILES: O=C(O)c1cc2nc(Cl)nc(N3CCOCC3)c2o1]

To a solution of 2-chloro-4-morpholinofuro[3,2-d]pyrimidine (2.4 g, 10 mmol) in anhydrous THF (40 mL) at −78° C. under N₂ was added n-BuLi (5.2 mL, 2.5 M, 13 mmol) dropwise. The reaction mixture was stirred at that temperature for 1 h. To the solution was added excessive amount of dry ice in one portion. The resulting reaction mixture was stirred at that temperature for 3 h. Upon the completion of the reaction as monitored by TLC, the reaction was quenched with water and the pH was adjusted to 5 using 1 N HCl aqueous solution. The aqueous solution was extracted with DCM (3×80 mL). The combined organic phase was dried over anhydrous Na₂SO₄, filtrated and concentrated under reduced pressure. The resulting residue was slurry in Et₂O to provide 2-chloro-4-morpholinofuro[3,2-d]pyrimidine-6-carboxylic acid (2.92 g, 10.3 mmol) as a yellow solid. LC-MS (ESI+): m/z 284/286 (MH⁺). ¹HNMR (300 MHz, CDCl₃) δ 7.58 (s, 1H), 4.04-3.95 (m, 4H), 3.78-3.76 (m, 4H).

### 1.2) Synthesis of 2-chloro-N-methyl-4-morpholinofuro[3, 2-d]pyrimidine-6-carboxamide

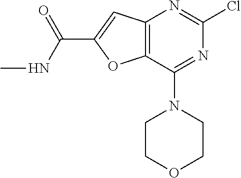 [SMILES: CNC(=O)c1cc2nc(Cl)nc(N3CCOCC3)c2o1]

To a solution of 2-chloro-4-morpholinofuro[3,2-d]pyrimidine-6-carboxylic acid (100 mg, 0.36 mmol) in DCM (10 mL) was added methanamine hydrochloride (13.5 mg, 0.2 mmol), EDCl (86 mg, 0.45 mmol) and DMAP (55 mg, 0.45 mmol). The reaction was stirred at room temperature overnight. Upon the completion of the reaction as monitored by TLC, the reaction was quenched with water (10 mL) and extracted with DCM (3×10 mL). The combined organic phase was dried over anhydrous Na₂SO₄, filtrated and concentrated under reduced pressure. The resulting residue was purified by silica gel column chromatography with a gradient elution of 1% MeOH/DCM to 2% MeOH/DCM to provide 2-chloro-N-methyl-4-morpholinofuro[3,2-d]pyrimidine-6-carboxamide (67 mg, 0.22 mmol) as white solid. LC-MS (ESI+): m/z 297/299 (MH⁺). ¹HNMR (300 MHz, CDCl₃) δ 7.61 (s, 1H), 6.35 (brs, 1H), 4.06-4.03 (m, 4H), 3.88-3.85 (m, 4H), 3.06 (d, J=5.1 Hz, 3H).

### 1.3) Synthesis of N-methyl-4-morpholino-2-(3-(m-tolyl)-1H-pyrazol-1-yl)furo[3,2-d] pyrimidine-6-carboxamide

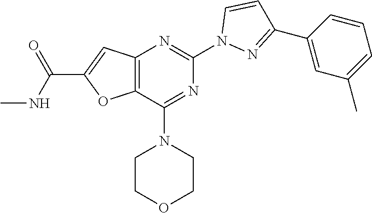 [SMILES: CNC(=O)c1cc2nc(-n3ccc(-c4cccc(C)c4)n3)nc(N3CCOCC3)c2o1]

To a solution of 2-chloro-N-methyl-4-morpholinofuro[3,2-d]pyrimidine-6-carboxamide (60 mg, 0.19 mmol) in DMF (4 mL) was added 3-(m-tolyl)-1H-pyrazole (35 mg, 0.23 mmol), Cs₂CO₃ (123 mg, 0.38 mmol) and CuI (8 mg, 0.038 mmol). The reaction was stirred at 110° C. overnight. Upon the completion of the reaction as monitored by TLC, the reaction was then cooled to room temperature, quenched with water (10 mL) and extracted with EtOAc (3×10 mL). The combined organic phase was dried over anhydrous Na₂SO₄, filtrated and concentrated under reduced pressure. The resulting residue was purified by preparative TLC to provide N-methyl-4-morpholino-2-(3-(m-tolyl)-1H-pyrazol-1-yl) furo[3,2-d]pyrimidine-6-carboxamide (15 mg, 0.036 mmol) as a white solid. LC-MS (ESI+): m/z 419 (MH⁺). ¹HNMR (300 MHz, DMSO-d₆) δ 8.85-8.82 (m, 1H), 8.70 (d, J=2.4 Hz, 1H), 7.79 (s, 1H), 7.74 (d, J=8.1 Hz, 1H), 7.54 (s, 1H), 7.36 (t, J=7.8 Hz, 1H), 7.20 (d, J=7.2 Hz, 1H), 7.02 (d, J=2.4 Hz, 1H), 4.12-4.08 (m, 4H), 3.86-3.82 (m, 4H), 2.86 (d, J=4.5 Hz, 3H), 2.39 (s, 3H).

### Example 26: Compound 98 Using General Synthetic Route 26

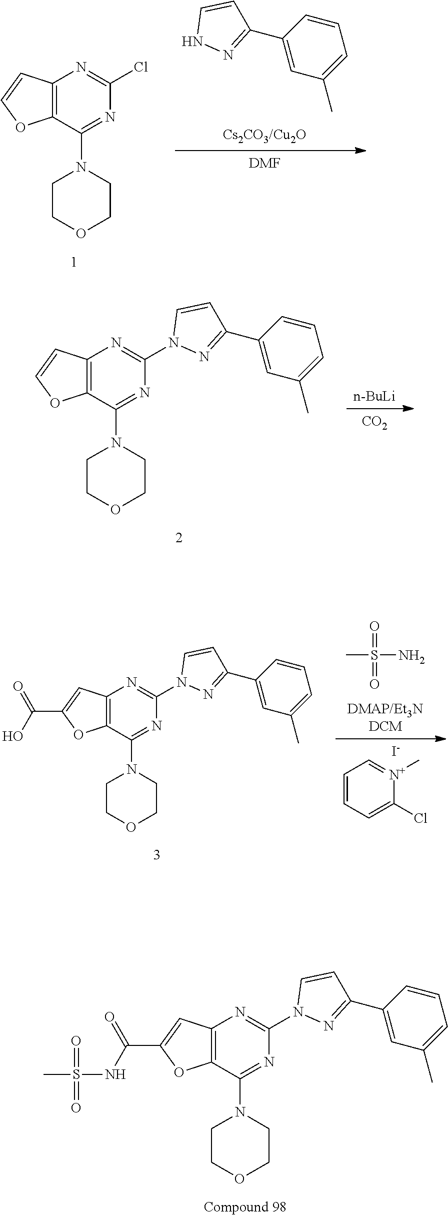

### 1.1) Synthesis of 4-morpholino-2-(3-(m-tolyl)-1H-pyrazol-1-yl)furo[3,2-d]pyrimidine

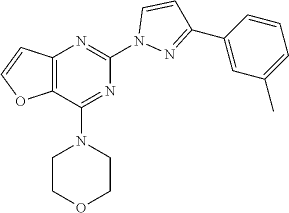 [SMILES: Cc1cccc(-c2ccn(-c3nc(N4CCOCC4)c4occc4n3)n2)c1]

To a solution of 2-chloro-4-morpholinofuro[3,2-d]pyrimidine (300 mg, 1.25 mmol) in DMF (4 mL) was added 3-(m-tolyl)-1H-pyrazole (237 mg, 0.35 mmol), Cs₂CO₃ (815 mg, 2.5 mmol) and CuI (24 mg, 0.125 mmol). The reaction was stirred at 110° C. overnight. Upon the completion of the reaction as monitored by TLC, the reaction was then cooled to room temperature, quenched with water (10 mL) and extracted with EtOAc (3×10 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 4-morpholino-2-(3-(m-tolyl)-1H-pyrazol-1-yl)furo[3,2-d]pyrimidine (269 mg, 0.75 mmol) as a white solid. LC-MS (ESI+): m/z 362 (MH⁺). ¹HNMR (300 MHz, DMSO-d₆) δ 8.75 (d, J=2.7 Hz, 1H), 8.38 (d, J=1.8 Hz, 1H), 7.82 (s, 1H), 7.77 (d, J=7.8 Hz, 1H), 7.36 (t, J=7.8 Hz, 1H), 7.21 (d, J=7.8 Hz, 1H), 7.13 (d, J=1.8 Hz, 1H), 7.07 (d, J=2.7 Hz, 1H), 4.12-4.06 (m, 4H), 3.92-3.86 (m, 4H), 2.40 (s, 3H).

### 1.2) Synthesis of 4-morpholino-2-(3-(m-tolyl)-1H-pyrazol-1-yl)furo[3,2-d]pyrimidine-6-carboxylic acid

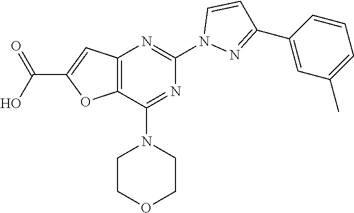 [SMILES: Cc1cccc(-c2ccn(-c3nc(N4CCOCC4)c4oc(C(=O)O)cc4n3)n2)c1]

To a solution of 4-morpholino-2-(3-(m-tolyl)-1H-pyrazol-1-yl)furo[3,2-d]pyrimidine (493 mg, 1.36 mmol) in anhydrous THF (20 mL) at −78° C. under N₂ was added n-BuLi (0.8 mL, 2.5 M, 2.0 mmol) dropwise. The reaction mixture was stirred at that temperature for 1 h. To the solution was added excessive amount of dry ice in one portion. The resulting reaction mixture was stirred at that temperature for 3 h. Upon the completion of the reaction as monitored by TLC, the reaction was quenched with water and the pH was adjusted to 5 using 1 N HCl aqueous solution. The aqueous solution was extracted with DCM/MeOH (15/1, 2×20 mL). The combined organic phase was dried over anhydrous Na₂SO₄, filtrated and concentrated under reduced pressure. The resulting residue was slurry in Et₂O to provide 4-morpholino-2-(3-(m-tolyl)-1H-pyrazol-1-yl)furo [3,2-d]pyrimidine-6-carboxylic acid (361 mg, 0.89 mmol) as a yellow solid. LC-MS (ESI+): m/z 406 (MH⁺). ¹HNMR (300 MHz, DMSO-d₆) δ 14.18 (s, 1H), 8.72 (d, J=2.7 Hz, 1H), 7.79-7.71 (m, 3H), 7.35 (t, J=7.5 Hz, 1H), 7.20 (d, J=7.2 Hz, 1H), 7.03 (d, J=2.4 Hz, 1H), 4.11-4.05 (m, 4H), 3.88-3.82 (m, 4H), 2.39 (s, 3H).

### 1.3) Synthesis of N-(methylsulfonyl)-4-morpholino-2-(3-(m-tolyl)-1H-pyrazol-1-yl)furo[3,2-d]pyrimidine-6-carboxamide

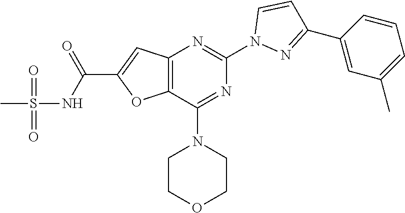 [SMILES: Cc1cccc(-c2ccn(-c3nc(N4CCOCC4)c4oc(C(=O)NS(C)(=O)=O)cc4n3)n2)c1]

To a solution of 4-morpholino-2-(3-(m-tolyl)-1H-pyrazol-1-yl)furo[3,2-d]pyrimidine-6-carboxylic acid (60 mg, 0.15 mmol) in DCM (10 mL) was added methanesulfonamide (28 mg, 0.30 mmol), 2-chloro-1-methylpyridinium iodide (45 mg, 0.18 mmol) and DMAP (1 mg, 0.007 mmol). The reaction was stirred at room temperature overnight. Upon the completion of the reaction as monitored by TLC, the reaction was quenched with water (10 mL) and extracted with DCM (3×10 mL). The combined organic phase was dried over anhydrous Na₂SO₄, filtrated and concentrated under reduced pressure. The resulting residue was purified by preparative TLC to provide N-(methylsulfonyl)-4-morpholino-2-(3-(m-tolyl)-1H-pyrazol-1-yl)furo[3,2-d]pyrimidine-6-carboxamide (14.2 mg, 0.03 mmol) as a yellow solid. LC-MS (ESI+): m/z 483 (MH⁺). ¹HNMR (300 MHz, CD₃OD) δ 8.61 (s, 1H), 7.79-7.69 (m, 2H), 7.61-7.59 (m, 1H), 7.50-7.18 (m, 2H), 6.84-6.80 (m, 1H), 4.25-4.14 (m, 4H), 3.91-3.85 (m, 4H), 3.16 (s, 3H), 2.37 (s, 3H).

### Example 27: Compound 127 Using General Synthetic Route 27

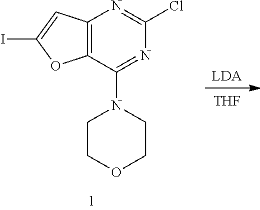 [SMILES: Clc1nc(N2CCOCC2)c2oc(I)cc2n1 ; "LDA" ; "THF"]

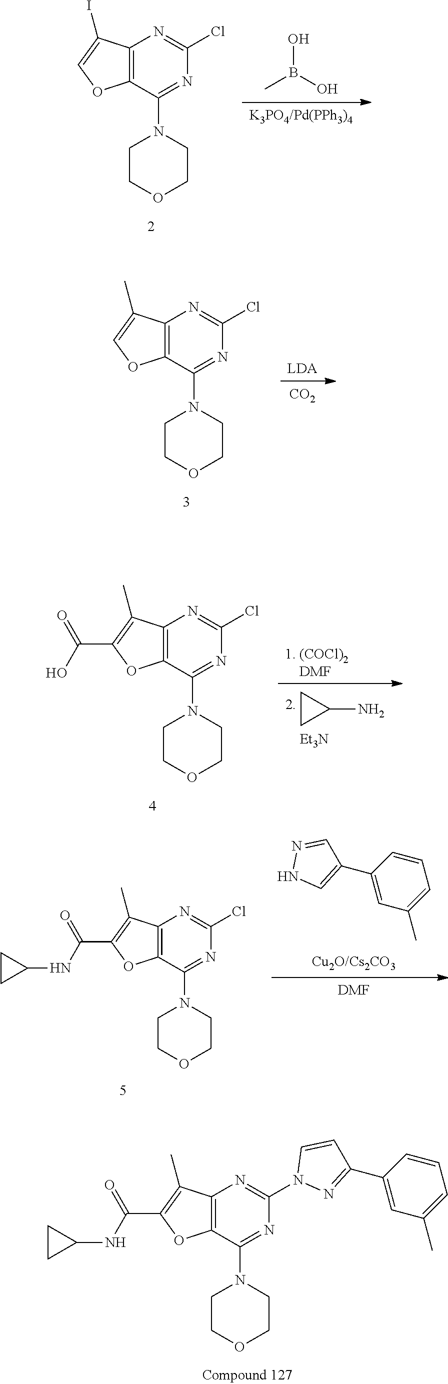

### 1.1) Synthesis of 2-chloro-7-iodo-4-morpholinofuro[3,2-d]pyrimidine

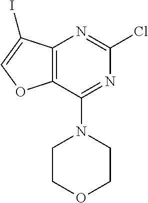 [SMILES: Clc1nc(N2CCOCC2)c2occ(I)c2n1]

To a solution of 2-chloro-6-iodo-4-morpholinofuro[3,2-d]pyrimidine (5.0 g, 13.7 mmol) in anhydrous THF (30 mL) at −78° C. under N₂ was added LDA (14 mL, 2 M, 27.4 mmol). The reaction mixture was stirred at −78° C. for 1 h. Upon the completion of the reaction as monitored by TLC, the reaction was quenched with H₂O (100 mL) and extracted with DCM (3×200 mL). The combined organic phase was dried over anhydrous Na₂SO₄, filtrated and concentrated under reduce pressure. The resulting residue was purified by silica gel column chromatography with a gradient elution of 10% EtOAc/PE to 30% EtOAc/PE to provide 2-chloro-7-iodo-4-morpholinofuro[3,2-d]pyrimidine (2.5 g, 6.85 mmol) as a white solid. LC-MS (ESI+): m/z 366/368 (MH⁺).¹HNMR (300 MHz, CDCl₃) δ 7.77 (s, 1H), 4.04-3.97 (m, 4H), 3.85-3.81 (m, 4H).

### 1.2) Synthesis of 2-chloro-7-methyl-4-morpholinofuro[3,2-d]pyrimidine

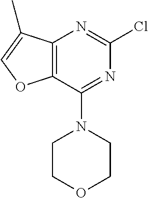 [SMILES: Cc1coc2c(N3CCOCC3)nc(Cl)nc12]

To a solution of 2-chloro-7-iodo-4-morpholinofuro[3,2-d]pyrimidine (50 mg, 0.14 mmol) in DME/H₂O (2/1, 3 mL) was added methylboronic acid (25 mg, 0.42 mmol), K₃PO₄ (44 mg, 0.21 mmol) and Pd(PPh₃)₄ (15 mg, 0.014 mmol). The reaction was stirred at 120° C. for 30 min under Microwave condition. Upon the completion of the reaction as monitored by TLC, the reaction was cooled to room temperature, quenched with water (30 mL) and extracted with EtOAc (2×10 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2-chloro-7-methyl-4-morpholinofuro[3,2-d]pyrimidine (35 mg, 0.14 mmol) as a white solid. LC-MS (ESI+): m/z 254/256 (MH⁺). ¹HNMR (300 MHz, CDCl₃) δ 7.53 (s, 1H), 4.04-3.98 (m, 4H), 3.86-3.82 (m, 4H), 2.22 (s, 3H).

### 1.3) Synthesis of 2-chloro-7-methyl-4-morpholinofuro[3,2-d]pyrimidine-6-carboxylic acid

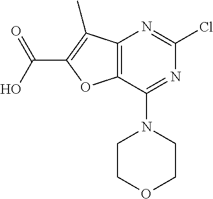 [SMILES: Cc1c(C(=O)O)oc2c(N3CCOCC3)nc(Cl)nc12]

To a solution of 2-chloro-6-iodo-7-methyl-4-morpholinofuro[3,2-d]pyrimidine (90 mg, 0.24 mmol) in anhydrous THF (100 mL) at −78° C. under N₂ was added n-BuLi (0.15 mL, 2.5 M, 0.36 mmol) dropwise. The reaction mixture was stirred at that temperature for 1 h. To the solution was added excessive amount of dry ice in one portion. The resulting reaction mixture was stirred at that temperature for 1 h. Upon the completion of the reaction as monitored by TLC, the reaction was quenched with water and the pH was adjusted to 5 using 1 N HCl aqueous solution. The aqueous solution was extracted with DCM (2×20 mL). The combined organic phase was dried over anhydrous Na₂SO₄, filtrated and concentrated under reduced pressure. The resulting residue was slurry in Et₂O to provide 2-chloro-7-methyl-4-morpholinofuro[3,2-d]pyrimidine-6-carboxylic acid (60 mg, 0.20 mmol) as a yellow solid. LC-MS (ESI+): m/z 298/300 (MH⁺).

### 1.4) Synthesis of 2-chloro-N-cyclopropyl-7-methyl-4-morpholinofuro[3,2-d]pyrimidine-6-carboxamide

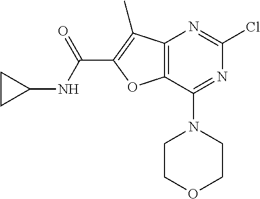 [SMILES: Cc1c(C(=O)NC2CC2)oc2c(N3CCOCC3)nc(Cl)nc12]

To a solution of 2-chloro-7-methyl-4-morpholinofuro[3,2-d]pyrimidine-6-carboxylic acid (60 mg, 0.20 mmol) in DCM (40 mL) was added oxalyl dichloride (52 mg, 0.4 mmol) and DMF (10 mg, 0.13 mmol). The reaction was stirred at room temperature for 2 h. Upon the completion of the reaction as monitored by TLC, the reaction solution was concentrated directly and the resulting residue was dissolved in DCM (20 mL). To the solution was added cyclopropanamine (23 mg, 0.4 mmol), followed by Et₃N (40 mg, 0.4 mmol) dropwise. The completion of the reaction was monitored by TLC. The reaction was quenched with water (20 mL) and extracted with EtOAc (2×10 mL). The combined organic phase was dried over anhydrous Na₂SO₄, filtrated and concentrated under reduced pressure. The resulting residue was purified by silica gel column chromatography with a gradient elution of 20% EtOAc/PE to 50% EtOAc/PE to provide 2-chloro-N-cyclopropyl-7-methyl-4-morpholinofuro[3,2-d]pyrimidine-6-carboxamide (54 mg, 0.16 mmol) as a white solid. LC-MS (ESI+): m/z 337/339 (MH⁺). ¹HNMR (300 MHz, CDCl₃) δ 6.37 (s, 1H), 4.03-3.99 (m, 4H), 3.87-3.84 (m, 4H), 2.88-2.83 (m, 1H), 2.55 (s, 3H), 0.98-0.92 (m, 2H), 0.71-0.65 (m, 2H).

### 1.5) Synthesis of N-cyclopropyl-7-methyl-4-morpholino-2-(4-(m-tolyl)-1H-pyrazol-1-yl)furo[3,2-d]pyrimidine-6-carboxamide

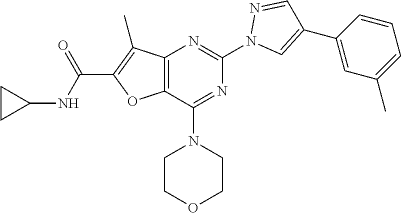 [SMILES: Cc1cccc(-c2cnn(-c3nc(N4CCOCC4)c4oc(C(=O)NC5CC5)c(C)c4n3)c2)c1]

To a solution of 2-chloro-N-cyclopropyl-7-methyl-4-morpholinofuro[3,2-d]pyrimidine-6-carboxamide (54 mg, 0.16 mmol) in DMF (3.0 mL) was added 4-(m-tolyl)-1H-pyrazole (50 mg, 0.32 mmol), Cs₂CO₃ (105 mg, 0.32 mmol) and Cu₂O (2.2 mg, 0.016 mmol). The reaction was stirred at 110° C. overnight. Upon the completion of the reaction as monitored by TLC, the reaction was quenched with water (10 mL) and a large amount of solid was precipitated. After filtration, the filtrate was concentrated directly and purified by flash silica gel column chromatography with a gradient elution of 20% EtOAc/PE to 50% EtOAc/PE to provide N-cyclopropyl-7-methyl-4-morpholino-2-(4-(m-tolyl)-1H-pyrazol-1-yl)furo[3,2-d]pyrimidine-6-carboxamide (25 mg, 0.054 mmol) as white solid. LC-MS (ESI+): m/z 459 (MH⁺). ¹HNMR (300 MHz, CDCl₃) δ 8.74 (s, 1H), 8.08 (s, 1H), 7.43-7.40 (m, 2H), 7.30-7.27 (m, 1H), 7.10 (d, J=6.9 Hz, 1H), 6.45 (s, 1H), 4.13-4.08 (m, 4H), 3.92-3.89 (m, 4H), 2.95-2.86 (m, 1H), 2.65 (s, 3H), 2.55 (s, 3H), 0.97-0.93 (m, 2H), 0.77-0.70 (m, 2H).

### Example 28: Compound 122 Using General Synthetic Route 28

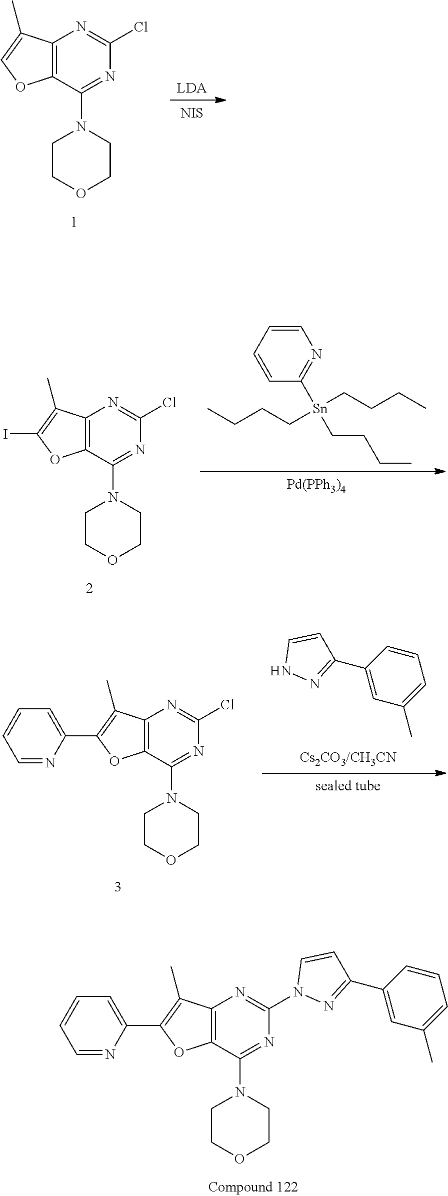

### 1.1) Synthesis of 2-chloro-6-iodo-7-methyl-4-morpholinofuro[3,2-d]pyrimidine

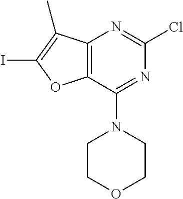 [SMILES: Cc1c(I)oc2c(N3CCOCC3)nc(Cl)nc12]

To a solution of 2-chloro-7-methyl-4-morpholinofuro[3,2-d]pyrimidine (350 mg, 1.38 mmol) in anhydrous THE (30 mL) at −78° C. under N₂ was added LDA (1.4 mL, 2 M, 2.76 mmol). After stirred at −78° C. for 1 h, to the solution was added a solution of NIS (374 mg, 1.66 mmol) in anhydrous THE (5 mL). Upon the completion of the reaction as monitored by TLC, the reaction was quenched with water (50 mL) and extracted with EtOAc (3×20 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2-chloro-6-iodo-7-methyl-4-morpholinofuro[3,2-d]pyrimidine (280 mg, 2.55 mmol) as yellow solid. LC-MS (ESI+): m/z 380/382 (MHI).

### 1.2) Synthesis of 2-chloro-7-methyl-4-morpholino-6-(pyridin-2-yl)furo[3,2-d]pyrimidine

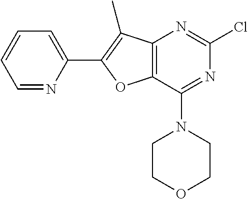 [SMILES: Cc1c(-c2ccccn2)oc2c(N3CCOCC3)nc(Cl)nc12]

A solution of 2-chloro-6-iodo-7-methyl-4-morpholinofuro[3,2-d]pyrimidine (200 mg, 0.53 mmol), 2-(tributylstannyl)pyridine (389 mg, 1.06 mmol) and Pd(PPh₃)₄ (61 mg, 0.053 mmol) in toluene (5 mL) was heated to 90° C. overnight. Upon the completion of the reaction as monitored by TLC, the reaction mixture was diluted with water and extracted with DCM/MeOH (15/1, 3×50 mL). The combined organic phase was dried over anhydrous Na₂SO₄, filtrated and concentrated under reduced pressure. The resulting residue was purified by silica gel column chromatography with a gradient elution of 30% EtOAc/PE to 50% EtOAc/PE to provide 2-chloro-7-methyl-4-morpholino-6-(pyridin-2-yl) furo[3,2-d]pyrimidine (70 mg, 0.21 mmol) as a white solid. LC-MS (ESI+): m/z 331/333 (MH⁺).

### 1.3) Synthesis of 7-methyl-4-morpholino-6-(pyridin-2-yl)-2-(3-(m-tolyl)-1H-pyrazol-1-yl)furo[3,2-d]pyrimidine

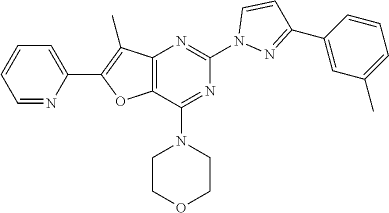 [SMILES: Cc1cccc(-c2ccn(-c3nc(N4CCOCC4)c4oc(-c5ccccn5)c(C)c4n3)n2)c1]

To a solution of 2-chloro-7-methyl-4-morpholino-6-(pyridin-2-yl)furo[3,2-d]pyrimidine (80 mg, 0.24 mmol) in CH₃CN (10 mL) was added 3-(m-tolyl)-1H-pyrazole (77 mg, 0.32 mmol) and Cs₂CO₃ (158 mg, 0.48 mmol). The reaction was stirred at 160° C. in a sealed tube overnight. The reaction mixture was then cooled to room temperature, diluted with water (10 mL) and extracted with DCM/MeOH (15/1, 3×20 mL). The combined organic phase was dried over anhydrous Na₂SO₄, filtrated and concentrated under reduced pressure. The resulting residue was purified by silica gel column chromatography with a gradient elution of 30% EtOAc/PE to 50% EtOAc/PE to provide 7-methyl-4-morpholino-6-(pyridin-2-yl)-2-(3-(m-tolyl)-1H-pyrazol-1-yl)furo [3,2-d]pyrimidine (9.2 mg, 0.02 mmol) as white solid. LC-MS (ESI+): m/z 453 (MH⁺). ¹HNMR (300 MHz, CDCl₃) δ 8.75 (d, J=4.2 Hz, 1H), 8.61 (d, J=2.1 Hz, 1H), 7.90 (s, 1H), 7.85-7.77 (m, 3H), 7.43-7.27 (m, 2H), 7.16 (d, J=7.5 Hz, 1H), 6.78 (s, 1H), 4.23-4.17 (m, 4H), 3.96-3.87 (m, 4H), 2.77 (s, 3H), 2.43 (s, 3H).

### Example 29: Compound 112 Using General Synthetic Route 29

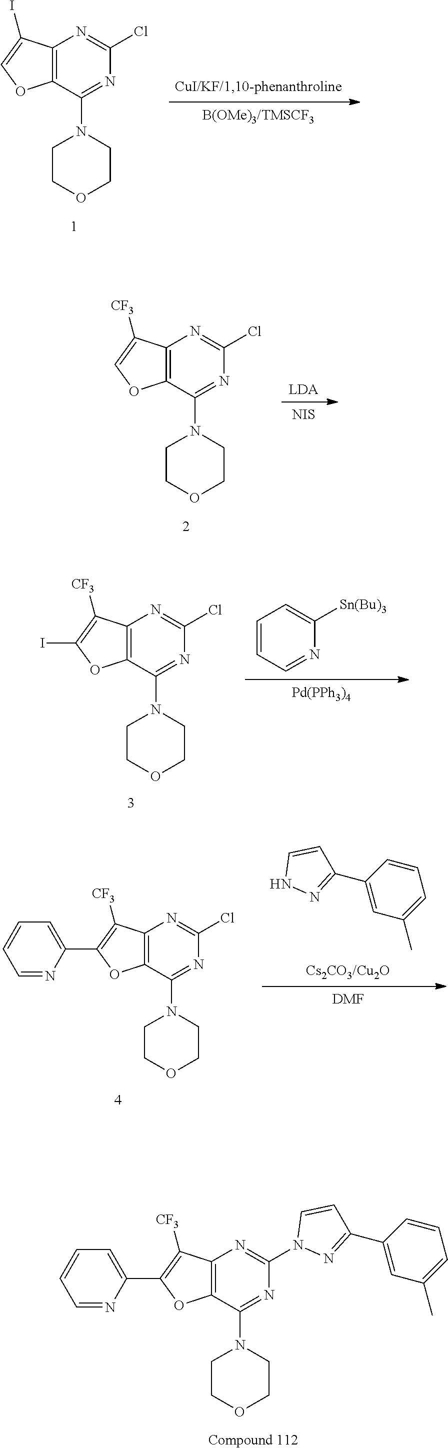

### 1.1) Synthesis of 2-chloro-4-morpholino-7-(trifluoromethyl)furo[3,2-d]pyrimidine

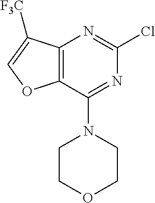 [SMILES: FC(F)(F)c1coc2c(N3CCOCC3)nc(Cl)nc12]

A solid mixture of 2-chloro-7-iodo-4-morpholinofuro[3,2-d]pyrimidine (300 mg, 0.82 mmol), KF (144 mg, 2.47 mmol), CuI (30 mg, 0.16 mmol) and 1,10-phenanthroline (30 mg, 0.16 mmol) in three neck flask was heated to 100° C. under reduced pressure using oil pump for 1 h. After being cooled to room temperature, to the mixture was added anhydrous DMSO (6.0 mL) to form a brown solution. To the solution was added B(OMe)₃ (252 mg, 2.47 mmol) and TMSCF₃ (348 mg, 2.47 mmol) dropwise. The reaction mixture was then heated to 55° C. After stirred at that temperature for 1 h, an additional TMSCF₃ (348 mg, 2.47 mmol) was added dropwise. Upon the completion of the reaction as monitored by TLC, the reaction was quenched with water (10 mL) and extracted with DCM (3×20 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2-chloro-4-morpholino-7-(trifluoromethyl)furo[3,2-d]pyrimidine (98 mg, 0.32 mmol) as a yellow solid. LC-MS (ESI+): m/z 308/310 (MH⁺).

### 1.2) Synthesis of 2-chloro-6-iodo-4-morpholino-7-(trifluoromethyl)furo[3,2-d]pyrimidine

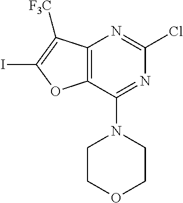 [SMILES: FC(F)(F)c1c(I)oc2c(N3CCOCC3)nc(Cl)nc12]

To a solution of 2-chloro-4-morpholino-7-(trifluoromethyl)furo[3,2-d]pyrimidine (160 mg, 0.52 mmol) in anhydrous THE (10 mL) at −78° C. under N₂ was added LDA (0.39 mL, 2 M, 0.78 mmol). After stirred at −78° C. for 1 h, to the solution was added a solution of NIS (141 mg, 0.63 mmol) in THE (10 mL). Upon the completion of the reaction as monitored by TLC, the reaction was quenched with water (25 mL) and extracted with DCM (3×15 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2-chloro-6-iodo-4-morpholino-7-(trifluoromethyl)furo[3,2-d] pyrimidine (148 mg, 0.34 mmol) as a white solid. LC-MS (ESI+): m/z 434/436 (MH⁺).

### 1.3) Synthesis of 2-chloro-4-morpholino-6-(pyridin-2-yl)-7-(trifluoromethyl)furo[3,2-d]pyrimidine

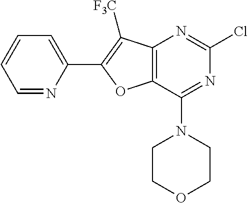 [SMILES: FC(F)(F)c1c(-c2ccccn2)oc2c(N3CCOCC3)nc(Cl)nc12]

A solution of 2-chloro-6-iodo-4-morpholino-7-(trifluoromethyl)furo[3,2-d]pyrimidine (100 mg, 0.23 mmol), 2-(tributylstannyl)pyridine (170 mg, 0.46 mmol) and Pd(PPh₃)₄ (13 mg, 0.023 mmol) in toluene (5 mL) was heated to 90° C. overnight. Upon the completion of the reaction as monitored by TLC, the reaction mixture was diluted with water and extracted with EtOAc (3×20 mL). The combined organic phase was dried over anhydrous Na₂SO₄, filtrated and concentrated under reduced pressure. The resulting residue was purified by silica gel column chromatography with a gradient elution of 20% EtOAc/PE to 40% EtOAc/PE to provide 2-chloro-4-morpholino-6-(pyridin-2-yl)-7-(trifluoromethyl)furo[3,2-d]pyrimidine (55 mg, 0.14 mmol) as a yellow solid. LC-MS (ESI+): m/z 385/387 (MH⁺).

### 1.4) Synthesis of 4-morpholino-6-(pyridin-2-yl)-2-(3-(m-tolyl)-1H-pyrazol-1-yl)-7-(trifluoromethyl)furo[3,2-d]pyrimidine

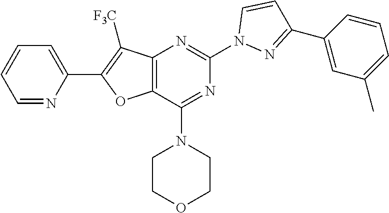 [SMILES: Cc1cccc(-c2ccn(-c3nc(N4CCOCC4)c4oc(-c5ccccn5)c(C(F)(F)F)c4n3)n2)c1]

To a solution of 2-chloro-4-morpholino-6-(pyridin-2-yl)-7-(trifluoromethyl) furo[3,2-d]pyrimidine (55 mg, 0.14 mmol) in DMF (6 mL) was added 3-(m-tolyl)-1H-pyrazole (45 mg, 0.29 mmol), Cs₂CO₃ (92 mg, 0.29 mmol) and Cu₂O (2 mg, 0.014 mmol). The reaction was stirred at 110° C. overnight. The reaction mixture was then diluted with water (10 mL) and extracted with EtOAc (3×20 mL). The combined organic phase was dried over anhydrous Na₂SO₄, filtrated and concentrated under reduced pressure. The resulting residue was purified by silica gel column chromatography with a gradient elution of 20% EtOAc/PE to 50% EtOAc/PE to provide 4-morpholino-6-(pyridin-2-yl)-2-(3-(m-tolyl)-1H-pyrazol-1-yl)-7-(trifluoromethyl)furo[3,2-d]pyrimidine (9.3 mg, 0.018 mmol) as a yellow solid. LC-MS (ESI+): m/z 507 (MH⁺). ¹HNMR (300 MHz, CDCl₃) δ 9.04 (d, J=4.2 Hz, 1H), 8.66 (d, J=2.1 Hz, 1H), 8.28-8.25 (m, 1H), 8.13-8.08 (m, 1H), 7.86-7.78 (m, 3H), 7.36-7.31 (m, 1H), 7.19 (d, J=7.2 Hz, 1H), 6.82 (s, 1H), 4.40-4.31 (m, 4H), 4.02-3.92 (m, 4H), 2.43 (s, 3H).

### Example 30: Compound 134 Using General Synthetic Route 30

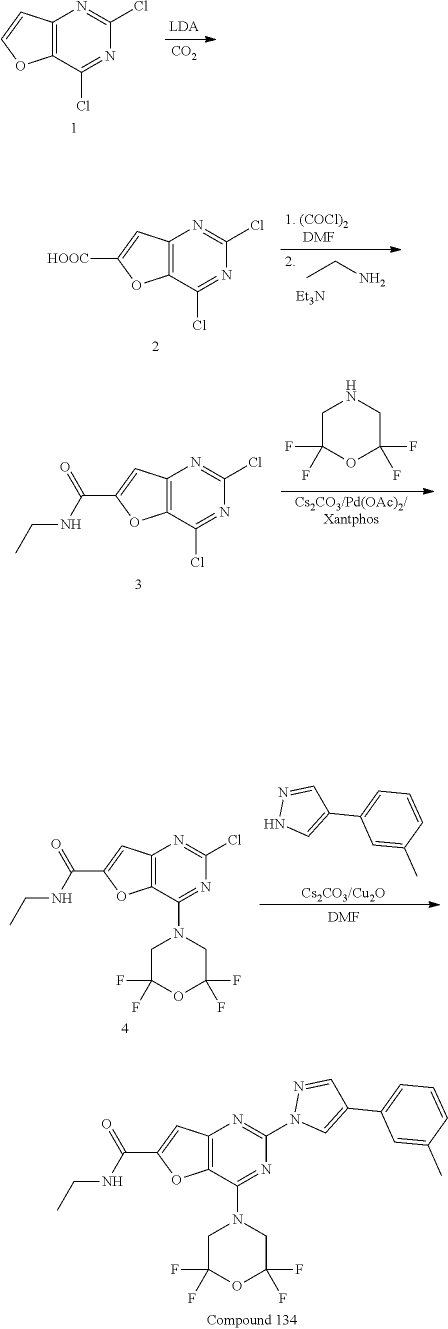

### 1.1) Synthesis of 2,4-dichlorofuro[3,2-d]pyrimidine-6-carboxylic acid

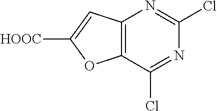 [SMILES: O=C(O)c1cc2nc(Cl)nc(Cl)c2o1]

To a solution of 2,4-dichlorofuro[3,2-d]pyrimidine (1 g, 5.29 mmol) in anhydrous THF (100 mL) at −78° C. under N₂ was added LDA (5.3 mL, 2 M, 10.6 mmol) dropwise. The reaction mixture was stirred at that temperature for 1 h. To the solution was added excessive amount of dry ice in one portion. The resulting reaction mixture was stirred at that temperature for 1 h. Upon the completion of the reaction as monitored by TLC, the reaction was quenched with water and the pH was adjusted to 5 using 1 N HCl aqueous solution. The aqueous solution was extracted with DCM (2×40 mL). The combined organic phase was dried over anhydrous Na₂SO₄, filtrated and concentrated under reduced pressure. The resulting residue was slurried in Et₂O to provide 2,4-dichlorofuro[3,2-d] pyrimidine-6-carboxylic acid (650 mg, 2.8 mmol) as a yellow solid. LC-MS (ESI+): m/z 233/235 (MH⁺).

### 1.2) Synthesis of 2,4-dichloro-N-ethylfuro[3,2-d]pyrimidine-6-carboxamide

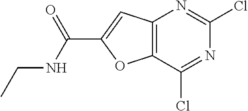 [SMILES: CCNC(=O)c1cc2nc(Cl)nc(Cl)c2o1]

To a solution of 2,4-dichlorofuro[3,2-d]pyrimidine-6-carboxylic acid (519 mg, 2.23 mmol) in DCM (40 mL) was added oxalyl dichloride (566 mg, 4.45 mmol) and DMF (20 mg, 0.27 mmol). The reaction was stirred at room temperature for 2 h. The completion of the reaction was monitored by TLC. The solution was concentrated directly without work-up. The resulting residue was dissolved in DCM (20 mL). To the solution was added ethanamine hydrochloride (218 mg, 2.67 mmol) and followed by Et₃N (450 mg, 4.45 mmol) dropwise. Upon the completion of the reaction as monitored by TLC, the reaction solution was concentrated directly and purified by silica gel column chromatography with a gradient elution of 10% EtOAc/PE to 30% EtOAc/PE to provide 2,4-dichloro-N— ethylfuro[3,2-d]pyrimidine-6-carboxamide (160 mg, 0.62 mmol) as a yellow solid. LC-MS (ESI+): m/z 260/262 (MH⁺). ¹HNMR (300 MHz, CDCl₃) δ 7.59 (s, 1H), 6.76 (brs, 1H), 3.61-3.54 (m, 2H), 1.31 (t, J=7.5 Hz, 3H).

### 1.3) Synthesis of 2-chloro-N-ethyl-4-(2,2,6,6-tetrafluoromorpholino)furo[3,2-d] pyrimidine-6-carboxamide

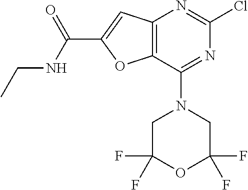 [SMILES: CCNC(=O)c1cc2nc(Cl)nc(N3CC(F)(F)OC(F)(F)C3)c2o1]

To a solution of 2,4-dichloro-N-ethylfuro[3,2-d]pyrimidine-6-carboxamide (200 mg, 0.77 mmol) in 1,4-dioxane/H₂O (2/1, 10 mL) under N₂ was added 2,2,6,6-tetrafluoromorpholine (110 mg, 0.69 mmol), Cs₂CO₃ (276 mg, 0.85 mmol), Pd(OAc)₂ (17 mg, 0.077 mmol) and Xantphos (45 mg, 0.077 mmol). The reaction mixture was stirred at 80° C. for 1 h. Upon the completion of the reaction as monitored by TLC, the reaction was quenched with water (30 mL) and extracted with EtOAc (2×20 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2-chloro-N-ethyl-4-(2,2,6,6-tetrafluoromorpholino)furo[3,2-d]pyrimidine-6-carboxamide (83 mg, 0.22 mmol) as a brown solid. LC-MS (ESI+): m/z 383/385 (MH⁺). ¹HNMR (300 MHz, CDCl₃) δ 7.61 (s, 1H), 6.36 (brs, 1H), 4.53-4.48 (m, 4H), 3.61-3.52 (m, 2H), 1.31 (t, J=7.5 Hz, 3H).

### 1.4) Synthesis of N-ethyl-4-(2,2,6,6-tetrafluoromorpholino)-2-(4-(m-tolyl)-1H-pyrazol-1-yl)furo[3,2-d]pyrimidine-6-carboxamide

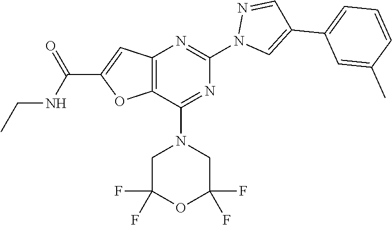 [SMILES: CCNC(=O)c1cc2nc(-n3cc(-c4cccc(C)c4)cn3)nc(N3CC(F)(F)OC(F)(F)C3)c2o1]

To a solution of 2-chloro-6-(pyridin-3-yl)-4-(2,2,6,6-tetrafluoromorpholino)furo [3,2-d]pyrimidine (59 mg, 0.15 mmol) in DMF (1 mL) was added 4-(m-tolyl)-1H-pyrazole (29 mg, 0.19 mmol), CS₂CO₃ (102 mg, 0.31 mmol) and Cu₂O (2 mg, 0.015 mmol). The reaction was stirred at 110° C. for 1 h. The solution was then cooled to room temperature and concentrated directly. The resulting residue was purified by preparative TLC to provide N-ethyl-4-(2,2,6,6-tetrafluoromorpholino)-2-(4-(m-tolyl)-1H-pyrazol-1-yl)furo[3,2-d]pyrimidine-6-carboxamide (11 mg, 0.022 mmol) as a brown solid. LC-MS (ESI+): m/z 505 (MH⁺). ¹HNMR (300 MHz, CDCl₃) δ 8.69 (s, 1H), 8.10 (s, 1H), 7.51 (s, 1H), 7.44-7.41 (m, 2H), 7.34-7.29 (m, 1H), 7.13 (d, J=7.5 Hz, 1H), 6.40-6.38 (m, 1H), 4.63-4.58 (m, 4H), 3.63-3.54 (m, 2H), 2.42 (s, 3H), 1.32 (t, J=7.2 Hz, 3H).

### Example 31: Compound 133 Using General Synthetic Route 31

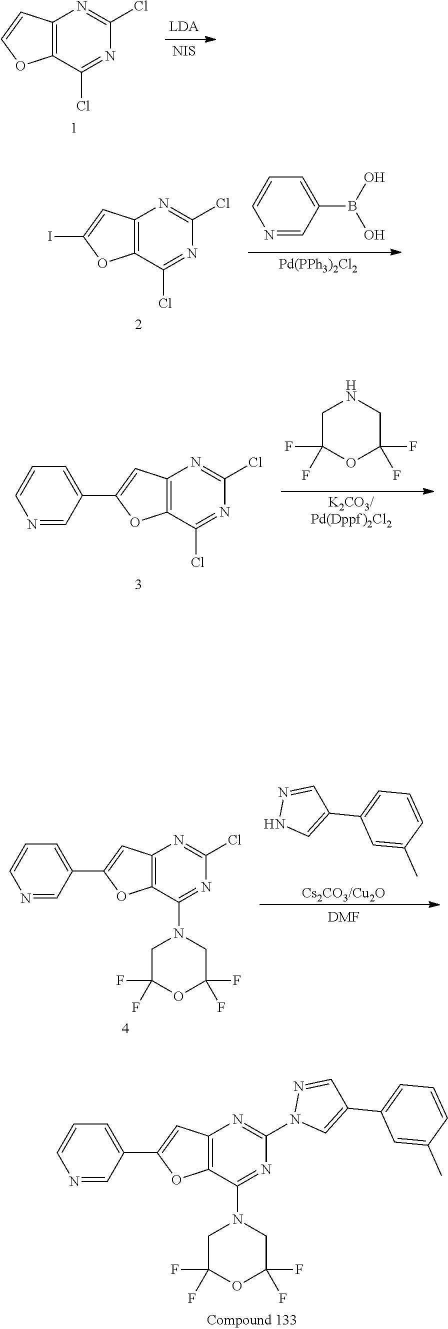

### 1.1) Synthesis of 2,4-dichloro-6-iodofuro[3,2-d]pyrimidine

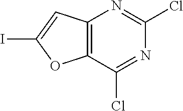 [SMILES: Clc1nc(Cl)c2oc(I)cc2n1]

To a solution of 2,4-dichlorofuro[3,2-d]pyrimidine (1.0 g, 5.32 mmol) in anhydrous THF (30 mL) at −78° C. under N₂ was added n-BuLi (5.33 mL, 2.5M, 13.3 mmol). After stirred at −78° C. for 1 h, to the solution was added a solution of NIS (1.44 g, 6.38 mmol) in anhydrous THF (10 mL). Upon the completion of the reaction as monitored by TLC, the reaction was quenched with water (50 mL) and extracted with DCM (3×50 mL). The combined organic phase was dried over anhydrous Na₂SO₄, filtrated and concentrated under reduced pressure. The resulting residue was purified by silica gel column chromatography with a gradient elution of 5% EtOAc/PE to 10% EtOAc/PE to provide 2,4-dichloro-6-iodofuro[3,2-d]pyrimidine (800 mg, 2.55 mmol) as yellow solid. LC-MS (ESI+): m/z 315/317 (MH⁺). ¹HNMR (300 MHz, CDCl₃) δ 7.21 (s, 1H).

### 1.2) Synthesis of 2,4-dichloro-6-(pyridin-3-yl)furo[3,2-d]pyrimidine

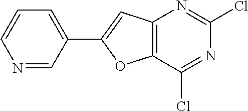 [SMILES: Clc1nc(Cl)c2oc(-c3cccnc3)cc2n1]

To a solution of 2,4-dichloro-6-iodofuro[3,2-d]pyrimidine (500 mg, 1.59 mmol) in 1,4-dioxane/H₂O (2/1, 30 mL) was added pyridin-3-ylboronic acid (156 mg, 1.27 mmol), K₂CO₃ (567 mg, 4.1 mmol) and PdCl₂(dppf) (117 mg, 0.16 mmol). The reaction was stirred at 100° C. for 1 h. Upon the completion of the reaction as monitored by TLC, the reaction was cooled to room temperature, quenched with water (30 mL) and extracted with EtOAc (4×10 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2,4-dichloro-6-(pyridin-3-yl)furo[3,2-d]pyrimidine (295 mg, 1.11 mmol) as a brown solid. LC-MS (ESI+): m/z 266/268 (MH⁺). ¹HNMR (300 MHz, CDCl₃) δ 9.21 (s, 1H), 8.78 (d, J=3.3 Hz, 1H), 8.27 (d, J=8.1 Hz, 1H), 7.74-7.71 (m, 1H), 7.21 (s, 1H).

### 1.3) Synthesis of 2-chloro-6-(pyridin-3-yl)-4-(2,2,6,6-tetrafluoromorpholino)furo [3,2-d]pyrimidine

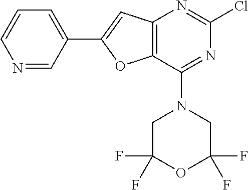 [SMILES: FC1(F)CN(c2nc(Cl)nc3cc(-c4cccnc4)oc23)CC(F)(F)O1]

To a solution of 2,4-dichloro-6-(pyridin-3-yl)furo[3,2-d]pyrimidine (117 mg, 0.43 mmol) in 1,4-dioxane/H₂O (2/1, 10 mL) under N₂ was added 2,2,6,6-tetrafluoromorpholine (63 mg, 0.39 mmol), Cs₂CO₃ (154 mg, 0.47 mmol), Pd(OAc)₂ (9 mg, 0.04 mmol) and Xantphos (27 mg, 0.04 mmol). The reaction was stirred at 80° C. for 1 h. Upon the completion of the reaction as monitored by TLC, the reaction was cooled to room temperature, quenched with water (30 mL) and extracted with DCM (4×10 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2-chloro-6-(pyridin-3-yl)-4-(2,2,6,6-tetrafluoromorpholino)furo[3,2-d]pyrimidine (97 mg, 0.25 mmol) as a brown solid. LC-MS (ESI+): m/z 389/391 (MH⁺). ¹HNMR (300 MHz, CDCl₃) δ 9.11 (s, 1H), 8.74 (d, J=3.9 Hz, 1H), 8.09 (d, J=7.2 Hz, 1H), 7.51-7.47 (m, 1H), 7.16 (s, 1H), 4.58-4.53 (m, 4H).

### 1.4) Synthesis of 6-(pyridin-3-yl)-4-(2,2,6,6-tetrafluoromorpholino)-2-(4-(m-tolyl)-1H-pyrazol-1-yl)furo[3,2-d]pyrimidine

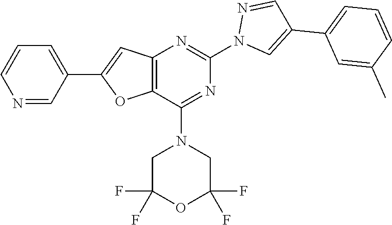 [SMILES: Cc1cccc(-c2cnn(-c3nc(N4CC(F)(F)OC(F)(F)C4)c4oc(-c5cccnc5)cc4n3)c2)c1]

To a solution of 2-chloro-6-(pyridin-3-yl)-4-(2,2,6,6-tetrafluoromorpholino) furo[3,2-d]pyrimidine (87 mg, 0.22 mmol) in DMF (1 mL) was added 4-(m-tolyl)-1H-pyrazole (43 mg, 0.27 mmol), Cs₂CO₃ (147 mg, 0.45 mmol) and Cu₂O (4 mg, 0.02 mmol). The reaction was stirred at 110° C. for 5 h. Upon the completion of the reaction as monitored by TLC, the reaction was cooled to room temperature, quenched with water (10 mL) and extracted with DCM/MeOH (15/1, 3×20 mL). The combined organic phase was dried over anhydrous Na₂SO₄, filtrated and concentrated under reduced pressure. The resulting residue was purified by preparative TLC to provide 6-(pyridin-3-yl)-4-(2,2,6,6-tetrafluoromorpholino)-2-(4-(m-tolyl)-1H-pyrazol-1-yl)furo[3,2-d]pyrimidine (7.5 mg, 0.014 mmol) as a white solid. LC-MS (ESI+): m/z 511 (MH⁺). ¹HNMR (300 MHz, CDCl₃) 9.14 (s, 1H), 8.74-8.71 (m, 2H), 8.13-8.11 (i, 2H), 7.52-7.42 (m, 3H), 7.34-7.32 (m, 2H), 7.13 (d, J=7.5 Hz, 1H), 4.67-4.62 (m, 4H), 2.42 (s, 3H).

Compounds 89 to 142 in Table 3 are made according to the procedures above.

TABLE 3

| | | | General | |
|---|---|---|---|---|
| | | | Synthetic | |
| Compound # | Name | Structure | Route | Data |
| 89 | 4-morpholino-6- (pyridin-4-yl)-2-(3- (m-tolyl)-1H- pyrazol-1- yl)furo[3,2- d]pyrimidine | 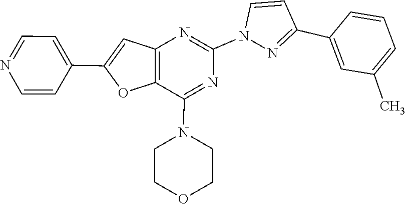 [SMILES: Cc1cccc(-c2ccn(-c3nc(N4CCOCC4)c4oc(-c5ccncc5)cc4n3)n2)c1] | 22 | LC-MS (ESI+): m/z 439 (MH⁺). ¹HNMR (300 MHz, DMSO-d₆) δ 8.78 (brs, 2H), 8.72 (s, 1H), 8.05-8.01 (m, 2H), 7.91 (s, 1H), 7.81 (s, 1H), 7.55 (d, J = 7.5 Hz, 1H), 7.36 (t, J = 7.5 Hz, 1H), 7.20 (d, J = 7.2 Hz, 1H), 7.03 (s, 1H), 4.18-4.12 (m, 4H), 3.90-3.84 (m, 4H), 2.40 (s, 3H). |
| 90 | 4-morpholino-6- (pyridin-3-yl)-2-(3- (m-toly))-1H- pyrazol-1- yl)furo[3,2- d]pyrimidine | 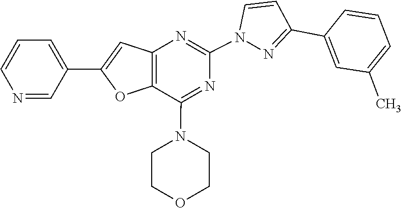 [SMILES: Cc1cccc(-c2ccn(-c3nc(N4CCOCC4)c4oc(-c5cccnc5)cc4n3)n2)c1] | 23 | LC-MS (ESI+): m/z 439 (MH⁺). ¹HNMR (300 MHz, DMSO-d₆) δ 9.30 (s, 1H), 8.71- 8.69 (m, 2H), 8.43 (d, J = 7.5 Hz, 1H), 7.81- 7.74 (m, 3H), 7.63-7.60 (m, 1H), 7.35(1, J = 7.5 Hz, 1H), 7.20 (d, 7 = 7.8 Hz, 1H), 7.02 (d, 7 = 2.7 Hz, 1H), 4.15- 4.11 (m, 4H), 3.90-3.82 (m, 4H), 2.41 (s, 3H). |
| 91 | 4-morpholino-6- (pyridin-2-yl)-2-(3- (m-tolyl)-1H- pyrazol-1- yl)furo[3,2- d]pyrimidine | 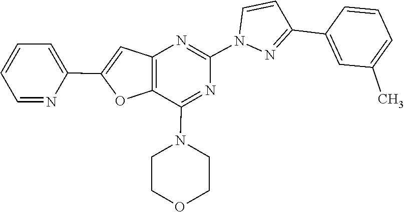 [SMILES: Cc1cccc(-c2ccn(-c3nc(N4CCOCC4)c4oc(-c5ccccn5)cc4n3)n2)c1] | 24 | LC-MS (ESI+): m/z 439 (MH⁺). ¹HNMR (300 MHz, DMSO-d₆) δ 8.76-8.71 (m, 2H), 8.12 (d, J = 7.5 Hz, 1H), 8.01 (t, J = 7.5 Hz, 1H), 7.80 (s, 1H), 7.75 (d, 7 = 7.2 Hz, 1H), 7.68 (s, 1H), 7.62-7.49 (m, 1H), 7.36 (t, 7 = 7.5 Hz, 1H), 7.20 (d, 7 = 7.2 Hz, 1H), 7.02 (s, 1H), 4.18-4.12 (m, 4H), 3.90-3.84 (m, 4H), 2.40 |
| | | | | (s, 3H). |
| 92 | N-methyl-4- morpholino-2-(3- (m-tolyl)-1H- pyrazol-1- yl)furo[3,2- d]pyrimidine-6- carboxamide | 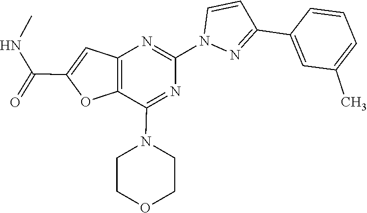 [SMILES: CNC(=O)c1cc2nc(-n3ccc(-c4cccc(C)c4)n3)nc(N3CCOCC3)c2o1] | 25 | LC-MS (ESI+): m/z 419 (MH⁺). ¹HNMR (300 MHz, DMSO-d₆) δ 8.85-8.82 (m, 1H), 8.70 (d, J = 2.4 Hz, 1H), 7.79 (s, 1H), 7.74 (d, J = 8.1 Hz, 1H), 7.54 (s, 1H), 7.36 (t, J = 7.8 Hz, 1H), 7.20 (d, J = 7.2 Hz, 1H), 7.02 (d, J = 2.4 Hz, 1H),4.12- 4.08 (m, 4H), 3.86-3.82 (m, 4H), 2.86 (d, J = 4.5 Hz, 3H), 2.39 (s, 3H). |
| 93 | N-(2- methoxyethyl)-4- morpholino-2-(3- (m-tolyl)-1H- pyrazol-1- yl)furo[3,2- d]pyrimidine-6- carboxamide | 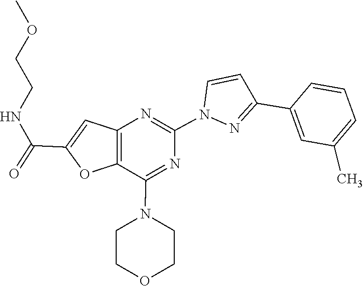 [SMILES: COCCNC(=O)c1cc2nc(-n3ccc(-c4cccc(C)c4)n3)nc(N3CCOCC3)c2o1] | 25 | LC-MS (ESI+): m/z 463 (MH⁺). ¹HNMR (300 MHz, CDCl₃) δ 8.53 (d, J = 2.4 Hz, 1H), 7.89 (s, 1H), 7.74 (d, J = 7.2 Hz, 1H), 7.52 (s, 1H), 7.35-7.29 (m, 1H), 7.16 (d, J = 7.5 Hz, 1H), 6.78-6.77 (m, 2H), 4.14-4.11 (m, 4H), 3.94-3.91 (m, 4H), 3.88-3.86 (m, 2H), 3.72-3.68 (m, 2H), 3.42 (s, 3H), 2.41 (s, 3H). |
| 94 | 4-morpholino-2-(3- (m-tolyl)-1H- pyrazol-1- yl)furo[3,2- d]pyrimidine-6- carboxamide | 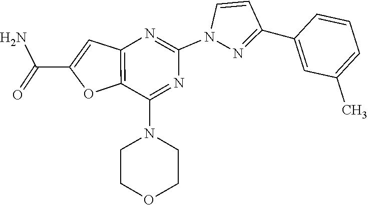 [SMILES: Cc1cccc(-c2ccn(-c3nc(N4CCOCC4)c4oc(C(N)=O)cc4n3)n2)c1] | 25 | LC-MS (ESI+): m/z 405 (MH⁺). ¹HNMR (300 MHz, DMSO-d₆) δ 8.71 (d, J = 2.7 Hz, 1H), 8.39 (s, 1H), 8.01 (s, 1H), 7.79 (s, 1H), 8.74 (d, J = 7.5 Hz, 1H), 7.56 (s, 1H), 7.35 (t, J = 7.5 Hz, 1H), 7.21 (d, J = 7.5 Hz, 1H), 7.02 (d, J = 2.7 Hz, 1H), 4.15-4.08 (m, 4H), 3.85-3.78 (m, 4H), 2.39 (s, 3H). |
| 95 | N-ethyl-4- morpholino-2-(3- (m-tolyl)-1H- pyrazol-1- yl)furo[3,2- d]pyrimidine-6- carboxamide | 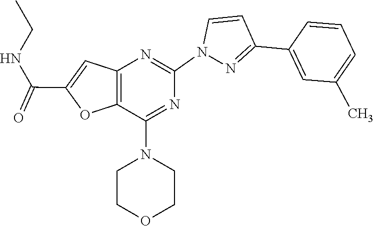 [SMILES: CCNC(=O)c1cc2nc(-n3ccc(-c4cccc(C)c4)n3)nc(N3CCOCC3)c2o1] | 25 | LC-MS (ESI+): m/z 433 (MH⁺). ¹HNMR (300 MHz, DMSO-d₆) δ 8.93-8.89 (m, 1H), 8.71 (d, J = 2.7 Hz, 1H), 7.79 (s, 1H), 7.75 (d, J = 7.5 Hz, 1H), 7.55 (s, 1H), 7.35 (t, J = 7.5 Hz, 1H), 7.20 (d, J = 7.5 Hz, 1H), 7.02 (d, J = 2.7 Hz, 1H),4.16- 4.08 (m, 4H), 3.85-3.78 (m, 4H), 3.43-3.33 (m, 2H), 2.39 (s, 3H), 1.17 (t, J = 6.9 Hz, 3H). |
| 96 | N-cyclopropyl-4- morpholino-2-(3- (m-tolyl)-1H- pyrazol-1- yl)furo[3,2- d]pyrimidine-6- carboxamide | 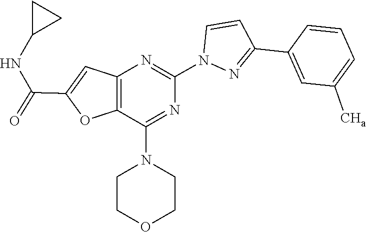 | 25 | LC-MS (ESI+): m/z 445 (MH⁺). ¹HNMR (300 MHz, DMSO-d₆) δ 8.89 (s, 1H), 8.70 (d, J = 2.4 Hz, 1H), 7.79 (s, 1H), 7.74(d, J = 7.2 Hz, 1H), 7.55 (s, 1H), 7.35 (t, J = 7.5 Hz, 1H), 7.20 (d, J = 7.2 Hz, 1H), 7.01 (d, J = 2.1 Hz, 1H), 4.15-4.08 (m, 4H), 3.85-3.80 (m, 4H), 2.87-2.81 (m, 1H), 2.39 (s, 3H), 0.83-0.78 (m, 2H), 0.69-0.64 (m, 2H). |
| 97 | N- (cyclopropylmethy 1)-4-morpholino-2- (3-(m-tolyl)-1H- pyrazol-1- yl)furo[3,2- d]pyrimidine-6- carboxamide | 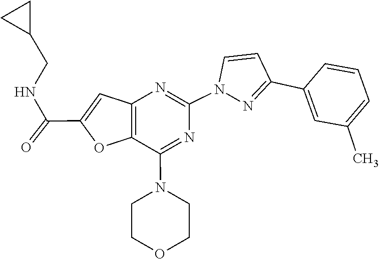 [SMILES: Cc1cccc(-c2ccn(-c3nc(N4CCOCC4)c4oc(C(=O)NCC5CC5)cc4n3)n2)c1] | 25 | LC-MS (ESI+): m/z 459 (MH⁺). ¹HNMR (300 MHz, DMSO-d₆) δ 8.99 (t, J = 5.4 Hz, 1H), 8.71 (d, J = 1.5 Hz, 1H), 7.79 (s, 1H), 7.74 (d, J = 8.1 Hz, 1H), 7.58 (s, 1H), 7.35 (t, J = 7.5 Hz, 1H), 7.20 (d, J = 7.5 Hz, 1H), 7.02 (d, J = 2.4 Hz, 1H), 4.15-4.08 (m, 4H), 3.85-3.80 (m, 4H), 3.21 (t, J = 6.0 Hz, 2H), 2.39 (s, 3H), 1.08-1.09 (m, 1H), 0.48-0.45 (m, 2H), 0.31-0.27 (m, 2H). |
| 98 | N- (methylsulfonyl)- 4-morpholino-2-(3- (m-tolyl)-1H- pyrazol-1- yl)furo[3,2- d]pyrimidine-6- carboxamide | 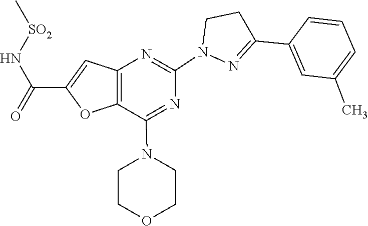 [SMILES: Cc1cccc(C2=NN(c3nc(N4CCOCC4)c4oc(C(=O)NS(C)(=O)=O)cc4n3)CC2)c1] | 26 | LC-MS (ESI+): m/z 483 (MH⁺). ¹HNMR (300 MHz, CD₃OD) δ 8.61 (s, 1H), 7.79-7.69 (m, 2H), 7.61-7.59 (m, 1H), 7.50-7.18 (m, 2H), 6.84-6.80 (m, 1H), 4.25-4.14 (m, 4H), 3.91-3.85 (m, 4H), 3.16 (s, 3H), 2.37 (s, 3H). |
| 99 | N-methoxy-4- morpholino-2-(3- (m-tolyl)-1H- pyrazol-1- yl)fur[3,2- d]pyrimidine-6- carboxamide | 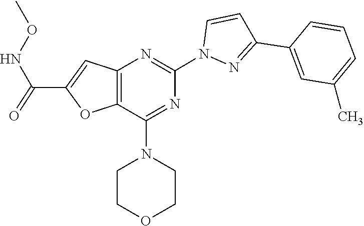 [SMILES: CONC(=O)c1cc2nc(-n3ccc(-c4cccc(C)c4)n3)nc(N3CCOCC3)c2o1] | 26 | LC-MS (ESI+): m/z 435 (MH⁺). ¹HNMR (300 MHz, CDCl₃) δ 9.72 (brs, 1H), 8.50 (d, J = 2.7 Hz, 1H), 7.82 (s, 1H), 7.70 (d, J = 8.1 Hz, 1H), 7.57 (s, 1H), 7.32-7.26 (m, 1H), 7.15 (d, J = 7.5 Hz, 1H), 6.77 (d, 7 = 2.7 Hz, 1H), 4.18-4.10 (m, 4H), 3.94-3.86 (m, 7H), 2.39 (s, 3H). |
| 100 | N-cyclopropyl-4- morpholino-2-(4- (m-tolyl)-1H- pyrazol-1- yl)furo[3,2- d]pyrimidine-6- carboxamide | 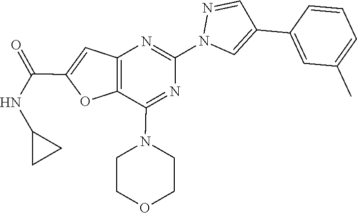 [SMILES: Cc1cccc(-c2cnn(-c3nc(N4CCOCC4)c4oc(C(=O)NC5CC5)cc4n3)c2)c1] | 25 | LC-MS (ESI+): m/z 445 (MH⁺). ¹HNMR (300 MHz, CDCl₃) δ 8.69 (s, 1H), 8.07 (s, 1H), 7.45-7.40 (m, 3H), 7.32-7.26 (m, 1H), 7.10 (d, J = 7.2 Hz, 1H), 6.45 (s, 1H), 4.16-4.11 (m, 4H), 3.97-3.92 (m, 4H), 2.91-2.90 (m, 1H), 2.41 (s, 3H), 0.96-0.94 (m, 2H), 0.75-0.70 (m, 2H). |
| 101 | N-ethyl-4- morpholino-2-(4- (m-tolyl)-1H- pyrazol-1- yl)furo[3,2- d]pyrimidine-6- carboxamide | 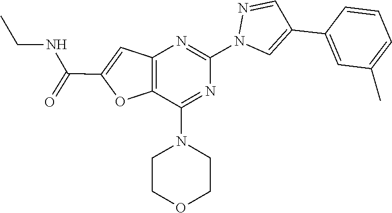 | 25 | LC-MS (ESI+): m/z 433 (MH⁺). ¹HNMR (300 MHz, CDCl₃) δ 8.72 (s, 1H),8.11(s, 1H), 7.43-7.40 (m, 3H), 7.33-7.26 (m, 1H), 7.10 (d, J = 7.5 Hz, 1H), 6.36 (brs, 1H), 4.17- 4.10 (m, 4H), 3.94-3.88 (m, 4H), 3.61-3.49 (m, 2H), 2.41 (s, 3H), 1.31 (t, J = 6.9 Hz, 3H). |
| 102 | 4-morpholino-6- (pyridin-3-yl)-2-(4- (m-tolyl)-1H- pyrazol-1- yl)furo[3,2- d]pyrimidine | 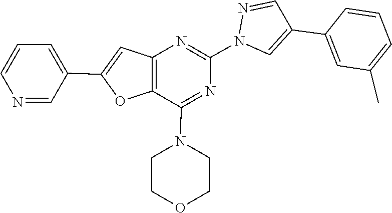 [SMILES: Cc1cccc(-c2cnn(-c3nc(N4CCOCC4)c4oc(-c5cccnc5)cc4n3)c2)c1] | 23 | LC-MS (ESI+): m/z 439 (MH⁺). ¹HNMR (300 MHz, CDCl₃) δ 9.10 (s, 1H), 8.72 (s, 1H), 8.69 (d, J = 3.6 Hz, 1H), 8.11-8.08 (m, 2H), 7.47- 7.41 (m, 3H), 7.33-7.30 (m, 1H), 7.21 (s, 1H), 7.10 (d, J = 7.2 Hz, 1H), 4.23-4.15 (m, 4H), 3.97- 3.89 (m, 4H), 2.41 (s, 3H). |
| 103 | 4-morpholino-6- (pyridin-2-yl)-2-(4- (m-tolyl)-1H- pyrazol-1- yl)fur[3,2- d]pyrimidine | 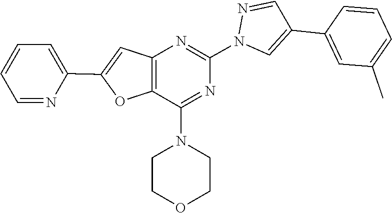 [SMILES: Cc1cccc(-c2cnn(-c3nc(N4CCOCC4)c4oc(-c5ccccn5)cc4n3)c2)c1] | 24 | LC-MS (ESI+): m/z 439 (MH⁺). ¹HNMR (300 MHz, DMSO-d₆) δ 9.03 (s, 1H), 8.76 (d, J = 4.2 Hz, 1H), 8.24 (s, 1,H), 8.16-8.13 (m, 1H), 8.05-8.00 (m, 1H), 7.62-7.50 (m, 4H), 7.31-7.27 (m, 1H), 7.09-7.07 (m, 1H), 4.19-4.12 (m, 4H), 3.87-3.82 (m, 4H), 2.36 (s, 3H). |
| 104 | 4-morpholino-N- (oxetan-3-yl)-2-(3- (m-tolyl)-1H- pyrazol-1- yl)furo[3,2- d]pyrimidine-6- carboxamide | 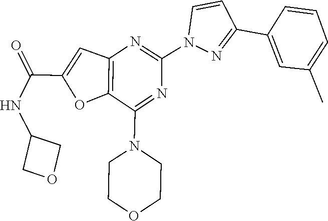 [SMILES: Cc1cccc(-c2ccn(-c3nc(N4CCOCC4)c4oc(C(=O)NC5COC5)cc4n3)n2)c1] | 26 | LC-MS (ESI+): m/z 461 (MH⁺). ¹HNMR (300 MHz, DMSO-d₆) δ 9.48 (d, J = 6.9 Hz, 1H), 8.72 (d, J = 2.4 Hz, 1H), 7.79 (s, 1H), 7.74 (d, J = 7.5 Hz, 1H), 7.64 (s, 1H), 7.36 (t, J = 7.8 Hz, 1H), 7.20 (d, J = 7.5 Hz, 1H), 7.03 (d, J = 2.4 Hz, 1H), 5.11-5.01 (m, 1H), 4.83 (t, J = 6.9 Hz, 2H), 4.66 (t, J = 6.3 Hz, 2H), |
| | | | | 4.15-4.08 (m, 4H), |
| | | | | 3.88-3.82 (m, 4H), |
| | | | | 2.39 (s, 3H). |
| 105 | 4-morpholino-N- (oxetan-3- ylmethyl)-2-(3-(m- tolyl)-1H-pyrazol- 1-yl)furo[3,2- d]pyrimidine-6- carboxamide | 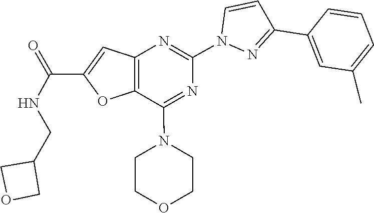 [SMILES: Cc1cccc(-c2ccn(-c3nc(N4CCOCC4)c4oc(C(=O)NCC5COC5)cc4n3)n2)c1] | 26 | LC-MS (ESI+): m/z 475 (MH⁺). ¹HNMR (300 MHz, DMSO-d₆) δ 9.03-8.99 (m, 1H), 8.72 (d, J = 2.4 Hz, 1H), 7.79 (s, 1H), 7.74 (d, J = 7.8 Hz, 1H), 7.58 (s, 1H), 7.36 (t, J = 7.8 Hz, 1H), 7.20 (d, J = 6.9 Hz, 1H), 7.02 (d, J = 2.7 Hz, 1H), 4.66 (t, J = 6.0 Hz, 2H), 4.38 (t, J = 6.0 Hz, 2H), 4.14- 4.08 (m, 4H), 3.86-3.81 |
| | | | | (m, 4H), 3.61 (t, J = 6.3 |
| | | | | Hz, 2H), 3.25-3.16 (m, |
| | | | | 1H), 2.39 (s, 3H). |
| 106 | 4-morpholino-N- (oxetan-3-yl)-2-(4- (m-tolyl)-1H- pyrazol-1- yl)furo[3,2- d]pyrimidine-6- carboxamide | 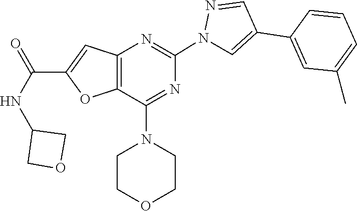 [SMILES: Cc1cccc(-c2cnn(-c3nc(N4CCOCC4)c4oc(C(=O)NC5COC5)cc4n3)c2)c1] | 26 | LC-MS (ESI+): m/z 461 (MH⁺). ¹HNMR (300 MHz, CDCl₃) δ 8.67 (s, 1H), 8.15 (s, 1H), 7.58 (s, 1H), 7.39- 7.32 (m, 2H), 7.29-7.27 (m, 1H), 7.16-7.12 (m, 1H), 5.31-5.23 (m, 1H), 5.03 (t, J = 6.9 Hz, 2H), 4.68 (t, J = 6.3 Hz, 2H), 4.18-4.12 (m, 4H), 3.95-3.89 (m, 4H), 2.41 (s, 3H). |
| 107 | 4-morpholino-N- (oxetan-3- ylmethyl)-2-(4-(m- tolyl)-1H-pyrazol- 1-yl)furo[3,2- d]pyrimidine-6- carboxamide | 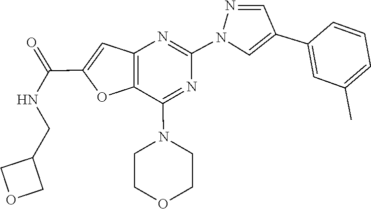 [SMILES: Cc1cccc(-c2cnn(-c3nc(N4CCOCC4)c4oc(C(=O)NCC5COC5)cc4n3)c2)c1] | 25 | LC-MS (ESI+): m/z 475 (MH⁺). ¹HNMR (300 MHz, CDCl₃) δ 8.69 (s, 1H), 8.11 (s, 1H), 7.48 (s, 1H), 7.39- 7.32 (m, 2H), 7.29-7.27 (m, 1H), 7.11 (d, J = 8.1 Hz, 1H), 6.76-6.74 (m, 1H), 4.88 (t, J = 6.9 Hz, 2H), 4.49 (t, J = 6.0 Hz, 2H), 4.16-4.08 (m, 4H), 3.94-3.86 (m, 4H), 3.81 (t, J = 5.7 Hz, 2H), 3.35-3.30 (m, 1H), 2.41 |
| | | | | (s, 3H). |
| 108 | N-(4- (dimethylamino) butyl)-4-morpholino- 2-(3-(m-tolyl)-1H- pyrazol-1- yl)furo[3,2- d]pyrimidine-6- carboxamide | 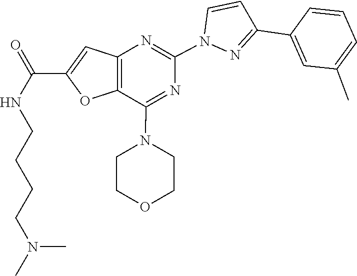 [SMILES: Cc1cccc(-c2ccn(-c3nc(N4CCOCC4)c4oc(C(=O)NCCCCN(C)C)cc4n3)n2)c1] | 25 | LC-MS (ESI+): m/z 504 (MH⁺). ¹HNMR (300 MHz, CDCl₃) δ 8.53 (d, J = 2.4 Hz, 1H), 8.40 (brs, 1H), 7.88 (s, 1H), 7.74 (d, J = 7.2 Hz, 1H), 7.60 (s, 1H), 7.33-7.29 (m, 1H), 7.15 (d, J = 7.5 Hz, 1H), 6.76 (d, J = 2.4 Hz, 1H), 4.25-4.15 (m, 4H), 3.97-3.86 (m, 4H), 3.56-3.51 (m, 2H), 3.11-3.02 (m, 2H), 2.82 (s, 6H), 2.40 (s, 3H), 1.98-1.96 (m, 2H), 1.88-1.85 (m, 2H). |
| 109 | N-(3- (dimethylamino) propyl)-4- morpholino-2-(3- (m-tolyl)-1H- pyrazol-1- yl)furo[3,2- d]pyrimidine-6- carboxamide | 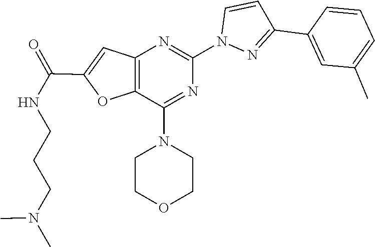 [SMILES: Cc1cccc(-c2ccn(-c3nc(N4CCOCC4)c4oc(C(=O)NCCCN(C)C)cc4n3)n2)c1] | 25 | LC-MS (ESI+): m/z 490 (MH⁺). ¹HNMR (300 MHz, CD₃OD) δ 8.93 (brs, 1H), 8.53 (d, J = 2.7 Hz, 1H), 7.89 (s, 1H), 7.74(d, J = 7.2 Hz, 1H), 7.61 (s, 1H), 7.33-7.31 (m, 1H), 7.15 (d, J = 7.5 Hz, 1H), 6.77 (d, 7 = 2.7 Hz, 1H), 4.20-4.15 (m, 4H), 3.97-3.88 (m, 4H), 3.67-3.64 (m, 2H), 2.91-2.82 (m, 2H), 2.58 (s, 6H), 2.41 (s, 3H), 2.01-1.96 (m, 2H). |
| 110 | N-(4- (dimethylamino) butyl)-4-morpholino- 2-(4-(m-tolyl)-1H- pyrazol-1- yl)furo[3,2- d]pyrimidine-6- carboxamide | 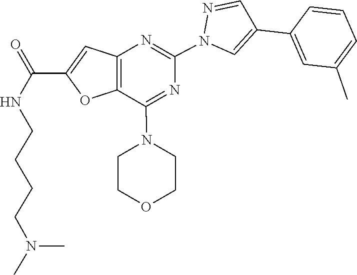 [SMILES: Cc1cccc(-c2cnn(-c3nc(N4CCOCC4)c4oc(C(=O)NCCCCN(C)C)cc4n3)c2)c1] | 25 | LC-MS (ESI+): m/z 504 (MH⁺). ¹HNMR (300 MHz, CDCl₃) δ 8.70 (s, 1H), 8.29 (brs, 1H), 8.06 (s, 1H), 7.51 (s, 1H), 7.42-7.40 (m, 2H), 7.32-7.29 (m, 1H), 7.10 (d, J = 7.5 Hz, 1H), 4.25-4.14 (m, 4H), 3.97-3.86 (m, 4H), 3.54-3.49 (m, 2H), 2.89-2.85 (m, 2H), 2.66 (s, 6H), 2.40 (s, 3H), 1.88-1.85 (m, 4H). |
| 111 | N-(3- (dimethylamino) propyl)-4- morpholino-2-(4- (m-tolyl)-1H- pyrazol-1- yl)furo[3,2- d]pyrimidine-6- carboxamide | 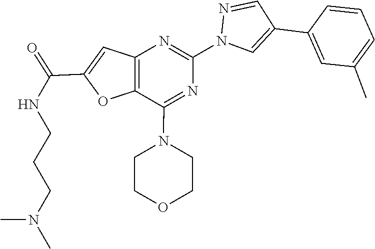 [SMILES: Cc1cccc(-c2cnn(-c3nc(N4CCOCC4)c4oc(C(=O)NCCCN(C)C)cc4n3)c2)c1] | 25 | LC-MS (ESI+): m/z 490 (MH⁺). ¹HNMR (300 MHz, CDCl₃) δ 8.90 (brs, 1H), 8.70 (s, 1H), 8.06 (s, 1H), 7.61- 7.43 (m, 3H), 7.32-7.29 (m, 1H), 7.10 (d, J = 7.5 Hz, 1H), 4.25-4.14 (m, 4H), 3.97-3.88 (m, 4H), 3.69-3.63 (m, 2H), 2.87-2.81 (m, 2H), 2.66 (s, 6H), 2.41 (s, 3H), 2.03-1.99 (m, 2H). |
| 112 | 4-morpholino-6- (pyridin-2-yl)-2-(3- (m-tolyl)-1H- pyrazol-1-yl)-7- (trifluoromethyl) furo[3,2- d]pyrimidine | 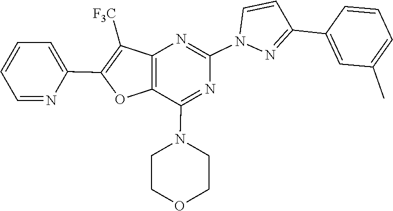 [SMILES: Cc1cccc(-c2ccn(-c3nc(N4CCOCC4)c4oc(-c5ccccn5)c(C(F)(F)F)c4n3)n2)c1] | 29 | LC-MS (ESI+): m/z 507 (MH⁺). ¹HNMR (300 MHz, CDCl₃) δ 9.04 (d, J = 4.2 Hz, 1H), 8.66 (d, J = 2.1 Hz, 1H), 8.28-8.25 (m, 1H), 8.13-8.08 (m, 1H), 7.86-7.78 (m, 3H), 7.36-7.31 (m, 1H), 7.19 (d, J = 7.2 Hz, 1H), 6.82 (s,1H), 4.40-4.31 (m, 4H), 4.02-3.92 (m, 4H), 2.43 (s, 3H). |
| 113 | (S)-N-(1- cyclopropylethyl)- 4-morpholino-2-(4- (m-tolyl)-1H- pyrazol-1- yl)furo[3,2- d]pyrimidine-6- carboxamide | 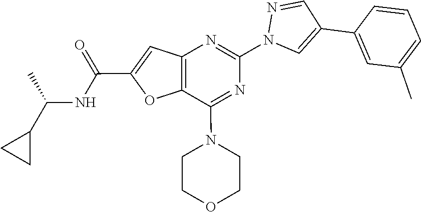 [SMILES: Cc1cccc(-c2cnn(-c3nc(N4CCOCC4)c4oc(C(=O)N[C@@H](C)C5CC5)cc4n3)c2)c1] | 25 | LC-MS (ESI+): m/z 473 (MH⁺). ¹HNMR (300 MHz, CDCl₃) δ 8.78 (s, 1H), 8.05 (s, 1H), 7.44-7.40 (m, 3H), 7.32-7.26 (m, 1H), 7.10 (d, J = 7.5 Hz, 1H), 6.31 (d, J = 7.8 Hz, 1H), 4.18-4.13 (m, 4H), 3.95-3.89 (m, 4H), 3.59-3.57 (m, 2H), 2.41 (s, 3H), 1.36 (d, J = 6.6 Hz, 3H), 1.03-0.96 (m, 1H), 0.63-0.32 (m, 4H). |
| 114 | (S)-N-(1- cyclopropylethyl)- 4-morpholino-2-(4- phenyl-1H- pyrazol-1- yl)furo[3,2- d]pyrimidine-6- carboxamide | 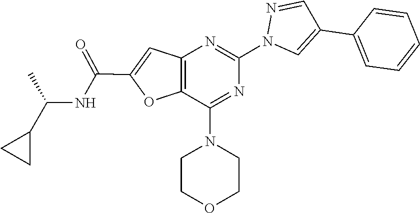 | 25 | LC-MS (ESI+): m/z 459 (MH⁺). ¹HNMR (300 MHz, CDCl₃) δ 8.72 (s, 1H), 8.08 (s, 1H), 7.61 (d, J = 7.5 Hz, 2H), 7.42-7.40 (m, 3H), 7.31-7.30 (m, 1H), 6.33 (d, J = 7.8 Hz, 1H), 4.18-4.13 (m, 4H), 3.97-3.91 (m, 4H), 3.63-3.57 (m, 2H), 1.36 (d, J = 6.6 Hz, 3H), 1.04-0.96 (m, 1H), 0.61-0.32 (m, 4H). |
| 115 | N- (cyclopropylmethyl)- 4-morpholino-2- (4-(m-tolyl)-1H- pyrazol-1- yl)furo[3,2- d]pyrimidine-6- carboxamide | 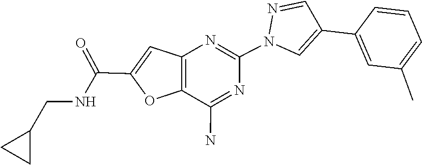 | 25 | LC-MS (ESI+): m/z 459 (MH⁺). ¹HNMR (300 MHz, CDCl₃) δ 8.70 (s, 1H), 8.07 (s, 1H), 7.46-7.40 (m, 3H), 7.32-7.30 (m, 1H), 7.10 (d, J = 7.5 Hz, 1H), 6.51-6.44 (m, 1H), 4.17-4.11 (m, 4H), 3.98-3.92 (m, 4H), 3.37 (t, J = 6.3 Hz, 2H), 2.41 |
| | | | | (s, 3H), 1.16-1.08 (m, |
| | | | | 1H), 0.64-0.55 (m, 2H), |
| | | | | 0.38-0.32 (m, 2H). |
| 116 | N- (cyclopropylmethy 1)-4-morpholino-2- (4-phenyl-1H- pyrazol-1- yl)furo[3,2- d]pyrimidine-6- carboxamide | 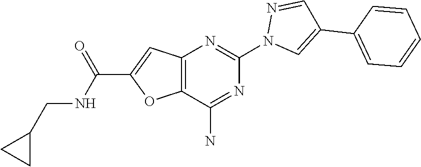 | 25 | LC-MS (ESI+): m/z 445 (MH⁺). ¹HNMR (300 MHz, CDCl₃) δ 8.72 (s, 1H), 8.09 (s, 1H), 7.61 (d, J = 7.2 Hz, 2H), 7.47-7.39 (m, 3H), 7.31-7.30 (m, 1H), 6.51-6.47 (m, 1H), 4.21-4.15 (m, 4H), 3.98-3.91 (m, 4H), 3.36 (t, J = 6.3 Hz, 2H), |
| | | | | 1.16-1.06 (m, 1H), |
| | | | | 0.64-0.57 (m, 2H), |
| | | | | 0.35-0.30 (m, 2H). |
| 117 | N-(2- methoxyethyl)-4- morpholino-2-(4- (m-tolyl)-1H- pyrazol-1- yl)fur[3,2- d]pyrimidine-6- carboxamide | 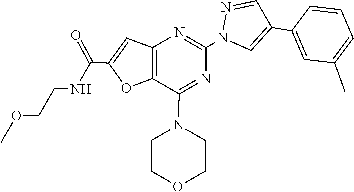 [SMILES: COCCNC(=O)c1cc2nc(-n3cc(-c4cccc(C)c4)cn3)nc(N3CCOCC3)c2o1] | 25 | LC-MS (ESI+): m/z 463 (MH⁺). ¹HNMR (300 MHz, CDCl₃) δ 8.70 (s, 1H), 8.08 (s, 1H), 7.49 (s, 1H), 7.42- 7.40 (m, 2H), 7.32-7.30 (m, 1H), 7.10 (d, J = 7.5 Hz, 1H), 6.85-6.83 (m, 1H), 4.19-4.13 (m, 4H), 3.97-3.91 (m, 4H), 3.72-3.67 (m, 2H), 3.61-3.51 (m, 2H), 3.42 (s, 3H), 2.41 (s, 3H). |
| 118 | N-(2- methoxyethyl)-4- morpholino-2-(4- phenyl-1H- pyrazol-1- yl)furo[3,2- d]pyrimidine-6- carboxamide | 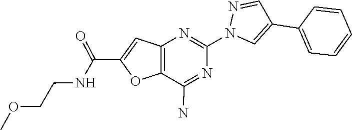 | 25 | LC-MS (ESI+): m/z 449 (MH⁺). ¹HNMR (300 MHz, CDCl₃) δ 8.72 (s, 1H), 8.08 (s, 1H), 7.62-7.60 (m, 2H), 7.49 (s, 1H), 7.44-7.39 (m, 2H), 7.32-7.30 (m, 1H), 6.83-6.74 (m, 1H), 4.23-4.13 (m, 4H), |
| | | | | 3.95-3.88 (m, 4H), |
| | | | | 3.70-3.67 (m, 2H), |
| | | | | 3.61-3.50 (m, 2H), 3.42 |
| | | | | (s, 3H). |
| 119 | N-(3- (methylsulfonyl) propyl)-4- morpholino-2-(4- (m-tolyl)-1H- pyrazol-1- yl)furo[3,2- d]pyrimidine-6- carboxamide | 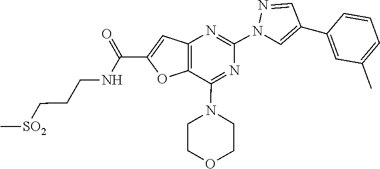 | 25 | LC-MS (ESI+): m/z 525 (MH⁺). ¹HNMR (300 MHz, CDCl₃) δ 8.70 (s, 1H), 8.08 (s, 1H), 7.61-7.40 (m, 4H), 7.32-7.30 (m, 1H), 7.10 (d, J = 7.2 Hz, 1H), 4.23-4.15 (m, 4H), 3.97-3.91 (m, 4H), 3.75-3.68 (m, 2H), 3.27-3.20 (m, 2H), 2.99 |
| | | | | (s, 3H), 2.41 (s, 3H), |
| | | | | 2.30-2.24 (m, 2H). |
| 120 | N-(3-(N,N- dimethylsulfamoyl) propyl)-4- morpholino-2-(4- (m-tolyl)-1H- pyrazol-1- yl)furo[3,2- d]pyrimidine-6- carboxamide | 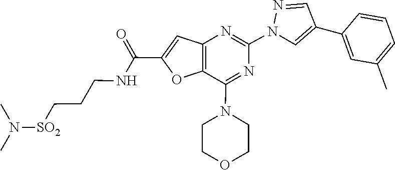 | 25 | LC-MS (ESI+): m/z 554 (MH⁺). ¹HNMR (300 MHz, CDCl₃) δ 8.70 (s, 1H), 8.07 (s, 1H), 7.62-7.58 (m, 1H), 7.43 (s, 1H), 7.42-7.40 (m, 2H), 7.32-7.26 (m, 1H), 7.10 (d, J = 7.5 Hz, 1H), 4.21-4.15 (m, 4H), 3.93-3.87 (m, 4H), 3.74-3.68 (m, 2H), |
| | | | | 3.08-3.04 (m, 2H), 2.96 |
| | | | | (s, 6H), 2.41 (s, 3H), |
| | | | | 2.25-2.21 (m, 2H). |
| 121 | N-(1- methylpiperidin-4- yl)-4-morpholino- 2-(4-(m-tolyl)-1H- pyrazol-1- yl)furo[3,2- d]pyrimidine-6- carboxamide | 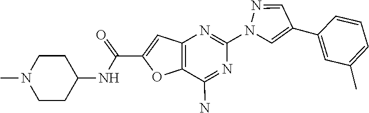 | 25 | LC-MS (ESI+): m/z 502 (MH⁺). ¹HNMR (300 MHz, CDCl₃) δ 8.69 (s, 1H), 8.07 (s, 1H), 7.61 (s, 1H), 7.47- 7.40 (m, 2H), 7.32-7.26 (m, 1H), 7.10 (d, J = 7.5 Hz, 1H), 6.42 (d, J = |
| | | | | 8.1 Hz, 1H), 4.16- |
| | | | | 4.03 (m, 5H), 3.94-3.87 |
| | | | | (m, 4H), 2.98-2.92 (m, |
| | | | | 2H), 2.40 (d, J = 4.5 |
| | | | | Hz, 6H), 2.32-2.24 (m, |
| | | | | 2H), 2.12-2.05 (m, 2H), |
| | | | | 1.76-1.65 (m, 2H). |
| 122 | 7-methyl-4- morpholino-6- (pyridin-2-yl)-2-(3- (m-tolyl)-1H- pyrazol-1- yl)furo[3,2- d]pyrimidine | 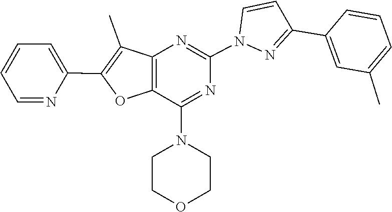 [SMILES: Cc1cccc(-c2ccn(-c3nc(N4CCOCC4)c4oc(-c5ccccn5)c(C)c4n3)n2)c1] | 28 | LC-MS (ESI+): m/z 453 (MH⁺). ¹HNMR (300 MHz, CDCl₃) δ 8.75 (d, J = 4.2 Hz, 1H), 8.61 (d, J = 2.1 Hz, 1H), 7.90 (s, 1H), 7.85-7.77 (m, 3H), 7.43-7.27 (m, 2H), 7.16 (d, J = 7.5 Hz, 1H), 6.78 (s, 1H), 4.23-4.17 (m, 4H), 3.96-3.87 (m, 4H), 2.77 (s, 3H), 2.43 (s, 3H). |
| 123 | N-(1- methylpiperidin-3- yl)-4-morpholino- 2-(4-(m-tolyl)-1H- pyrazol-1- yl)furo[3,2- d]pyrimidine-6- carboxamide | 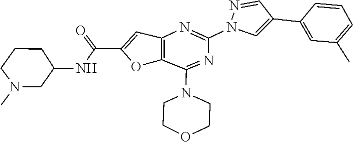 [SMILES: Cc1cccc(-c2cnn(-c3nc(N4CCOCC4)c4oc(C(=O)NC5CCCN(C)C5)cc4n3)c2)c1] | 25 | LC-MS (ESI+): m/z 502 (MH⁺). ¹HNMR (300 MHz, CD₃OD) δ 8.83 (s, 1H), 8.12 (s, 1H), 7.48-7.43 (m, 3H), 7.27 (t, J = 7.5 Hz, 1H), 7.08 (d, J = 7.5 Hz, 1H), 4.22-4.13 (m, 5H), 3.94-3.86 (m, 4H), |
| | | | | 3.07-3.01 (m, 1H), |
| | | | | 2.83-2.78 (m, 1H), 2.39 |
| | | | | (d, J = 4.5 Hz, 6H), |
| | | | | 2.28-2.23 (m, 2H), |
| | | | | 1.97-1.84 (m, 2H), |
| | | | | 1.74-1.70 (m, 1H), |
| | | | | 1.54-1.50 (m, 1H). |
| 124 | N-(1- methylpyrrolidin- 3-yl)-4- morpholino-2-(4- (m-tolyl)-1H- pyrazol-1- yl)furo[3,2- d]pyrimidine-6- carboxamide | 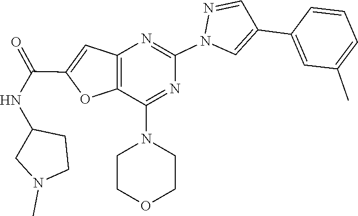 [SMILES: Cc1cccc(-c2cnn(-c3nc(N4CCOCC4)c4oc(C(=O)NC5CCN(C)C5)cc4n3)c2)c1] | 25 | LC-MS (ESI+): m/z 488 (MH⁺). ¹HNMR (300 MHz, CD₃OD) δ 8.88 (s, 1H), 8.14 (s, 1H), 7.51-7.45 (m, 3H), 7.27 (t, J = 7.2 Hz, 1H), 7.10 (d, J = 8.1 Hz, 1H), 4.67-4.61 (m, 1H), 4.25-4.18 (m, 4H), 3.91-3.85 (m, 4H), 3.50-3.26 (m, 3H), 3.13-3.03 (m, 1H), 2.78 (s, 3H), 2.54-2.49 (m, 1H), 2.38 (s, 3H), 2.19- |
| | | | | 2.13 (m, 1H). |
| 125 | 4-morpholino-N- (tetrahydro-2H- pyran-4-yl)-2-(4- (m-tolyl)-1H- pyrazol-1- yl)furo[3,2- d]pyrimidine-6- carboxamide | 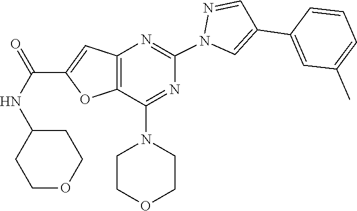 [SMILES: Cc1cccc(-c2cnn(-c3nc(N4CCOCC4)c4oc(C(=O)NC5CCOCC5)cc4n3)c2)c1] | 25 | LC-MS (ESI+): m/z 489 (MH⁺). ¹HNMR (300 MHz, CDCl₃) δ 8.70 (s, 1H), 8.08 (s, 1H), 7.61-7.40 (m, 3H), 7.32-7.26 (m, 1H), 7.10 (d, J = 7.8 Hz, 1H), 6.20 (d, J = 6.6 Hz, 1H), 4.31-4.23 (m, 1H), 4.15-4.11 (m, 4H), 4.05-4.01 (m, 2H), 3.99-3.92 (m, 4H), 3.55 (d, J = 11.4 Hz, 2H), 2.41 (s, 3H), 2.10-2.02 |
| | | | | (m, 2H), 1.74-1.58 (m, 2H). |
| 126 | 4-morpholino-N- (tetrahy drofuran-3- yl)-2-(4-(m-tolyl)- 1H-pyrazol-1- yl)furo[3,2- d]pyrimidine-6- carboxamide | 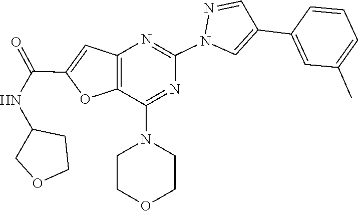 [SMILES: Cc1cccc(-c2cnn(-c3nc(N4CCOCC4)c4oc(C(=O)NC5CCOC5)cc4n3)c2)c1] | 25 | LC-MS (ESI+): m/z 475 (MH⁺). ¹HNMR (300 MHz, CDCl₃) δ 8.69 (s, 1H), 8.07 (s, 1H), 7.47 (s, 1H), 7.42- 7.40 (m, 2H), 7.32-7.26 (m, 1H), 7.10 (d, J = 7.2 Hz, 1H), 6.51 (d, J = 7.2 Hz, 1H), 4.84- 4.77 (m, 1H), 4.18-4.12 (m, 4H), 4.09-4.01 (m, 1H), 3.94-3.80 (m, 7H), 2.48-2.37 (m, 4H), 2.03-1.97 (m, 1H). |
| 127 | N-cyclopropyl-7- methyl-4- morpholino-2-(4- (m-tolyl)-1H- pyrazol-1- yl)furo[3,2- d]pyrimidine-6- carboxamide | 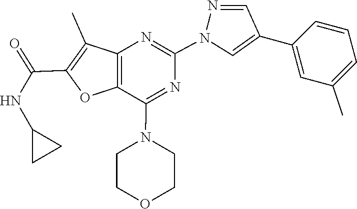 [SMILES: Cc1cccc(-c2cnn(-c3nc(N4CCOCC4)c4oc(C(=O)NC5CC5)c(C)c4n3)c2)c1] | 27 | LC-MS (ESI+): m/z 459 (MH⁺). ¹HNMR (300 MHz, CDCl₃) δ 8.74 (s, 1H), 8.08 (s, 1H), 7.43-7.40 (m, 2H), 7.30-7.27 (m, 1H), 7.10 (d, J = 6.9 Hz, 1H), 6.45 (s, 1H), 4.13-4.08 (m, 4H), 3.92-3.89 (m, 4H), 2.95-2.86 (m, 1H), 2.65 (s, 3H), 2.55 (s, 3H), 0.97-0.93 (m, 2H), 0.77-0.70 (m, 2H). |
| 128 | N-(2-(N,N- dimethylsulfamoyl )ethyl)-4- morpholino-2-(4- (m-tolyl)-1H- pyrazol-1- yl)furo[3,2- d]pyrimidine-6- carboxamide | 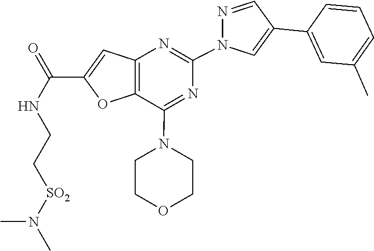 [SMILES: Cc1cccc(-c2cnn(-c3nc(N4CCOCC4)c4oc(C(=O)NCCS(=O)(=O)N(C)C)cc4n3)c2)c1] | 25 | LC-MS (ESI+): m/z 540 (MH⁺). ¹HNMR (300 MHz, CDCl₃) δ 8.70 (s, 1H), 8.07 (s, 1H), 7.69-7.61 (m, 1H), 7.52 (s, 1H), 7.42-7.40 (m, 2H), 7.32-7.30 (m, 1H), 7.10 (d, J = 7.8 Hz, 1H), 4.18-4.13 (m, 4H), 4.05-3.99 (m, 2H), 3.93-3.87 (m, 4H), 3.17-3.13 (m, 2H), 2.93 (s, 6H), 2.41 (s, 3H). |
| 129 | N-(1- methylazepan-4- yl)-4-morpholino- 2-(4-(m-tolyl)-1H- pyrazol-1- yl)furo[3,2- d]pyrimidine-6- carboxamide | 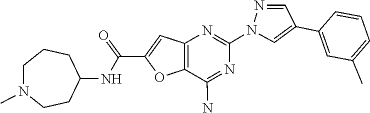 | 25 | LC-MS (ESI+): m/z 516 (MH⁺). ¹HNMR (300 MHz, CDCl₃) δ 8.71 (s, 1H), 8.07 (s, 1H), 7.76 (d, J = 8.4 Hz, 1H), 7.49-7.40 (m, 3H), 7.32-7.26 (m, 1H), 7.10 (d, J = 7.8 Hz, |
| | | | | 1H), 4.71-4.63 (m, 1H), |
| | | | | 4.21-4.13 (m, 4H), |
| | | | | 3.94-3.87 (m, 4H), |
| | | | | 2.86-2.70 (m, 2H), |
| | | | | 2.57-2.47 (m, 2H), 2.41 |
| | | | | (s, 6H), 2.11-1.82 (m, |
| | | | | 3H), 1.75-1.67 (m, 3H). |
| 130 | N-(1- methylazocan-3- yl)-4-morpholino- 2-(4-(m-tolyl)-1H- pyrazol-1- yl)furo[3,2- d]pyrimidine-6- carboxamide | 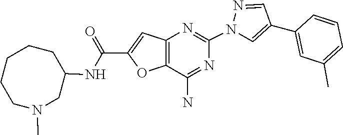 | 25 | LC-MS (ESI+): m/z 516 (MH⁺). ¹HNMR (300 MHz, CDCl₃) δ 8.70 (s, 1H), 8.08 (s, 1H), 7.86 (d, J = 4.5 Hz, 1H), 7.61-7.36 (m, 3H), 7.32-7.26 (m, 1H), 7.10 (d, J = 7.8 Hz, 1H), 4.31-4.13 (m, 5H), |
| | | | | 3.95-3.89 (m, 4H), |
| | | | | 2.89-2.82 (m, 2H), |
| | | | | 2.66-2.57 (m, 1H), |
| | | | | 2.51-2.41 (m, 7H), |
| | | | | 2.11-2.01 (m, 1H), |
| | | | | 1.87-1.81 (m, 1H), |
| | | | | 1.80-1.62 (m, 4H). |
| 131 | N-ethyl-4- morpholino(dg)-2- (4-(m-tolyl)-1H- pyrazol-1- yl)furo[3,2- d]pyrimidine-6- carboxamide | 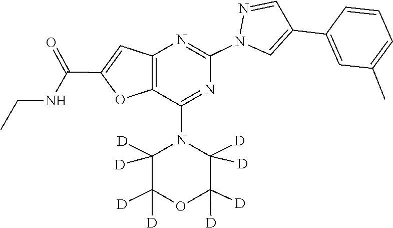 [SMILES: [2H]C1([2H])OC([2H])([2H])C([2H])([2H])N(c2nc(-n3cc(-c4cccc(C)c4)cn3)nc3cc(C(=O)NCC)oc23)C1([2H])[2H]] | 30 | LC-MS (ESI+): m/z 441 (MH⁺). ¹HNMR (300 MHz, DMSO-d₆) δ 8.98 (s, 1H), 8.95- 8.90 (m, 1H), 8.23 (s, 1H), 7.62-7.56 (m, 2H), 7.51 (s, 1H), 7.29 (t, J = 7.5 Hz, 1H), 7.08 (d, J = 7.5 Hz, 1H), 3.44- 3.32 (m, 2H), 2.36 (s, 3H), 1.16 (t, J = 7.2 Hz, 3H). |
| 132 | 4-morpholino (dg)- 6-(pyridin-3-yl)-2- (4-(m-tolyl)-1H- pyrazol-1- yl)furo[3,2- d]pyrimidine | 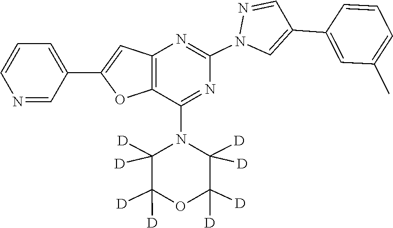 [SMILES: [2H]C1([2H])OC([2H])([2H])C([2H])([2H])N(c2nc(-n3cc(-c4cccc(C)c4)cn3)nc3cc(-c4cccnc4)oc23)C1([2H])[2H]] | 31 | LC-MS (ESI+): m/z 447 (MH⁺). ¹HNMR (300 MHz, DMSO-d₆) δ 9.29 (s, 1H), 9.01 (s, 1H), 8.69 (d, J = 4.2 Hz, 1H), 8.43 (d, J = 7.8 Hz, 1H), 8.23 (s, 1H), 7.80 (s, 1H), 7.62- 7.56 (m, 3H), 7.29 (t, J = 7.5 Hz, 1H), 7.08 (d, J = 7.2 Hz, 1H), 2.36 (s, 3H). |
| 133 | 6-(pyridin-3-yl)-4- (2,2,6,6- tetrafluoromorphol ino)-2-(4-(m- tolyl)-1H-pyrazol- 1-yl)furo[3,2- d]pyrimidine | 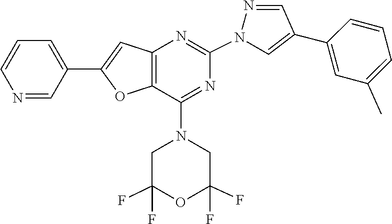 [SMILES: Cc1cccc(-c2cnn(-c3nc(N4CC(F)(F)OC(F)(F)C4)c4oc(-c5cccnc5)cc4n3)c2)c1] | 31 | LC-MS (ESI+): m/z 511 (MH⁺). ¹HNMR (300 MHz, CDCl₃) δ 9.14 (s, 1H), 8.74-8.71 (m, 2H), 8.13-8.11 (m, 2H), 7.52-7.42 (m, 3H), 7.34-7.32 (m, 2H), 7.13 (d, J = 7.5 Hz, 1H), 4.67-4.62 (m, 4H), 2.42 (s, 3H). |
| 134 | N-ethyl-4-(2,2,6,6- tetrafluoromorphol ino)-2-(4-(m- tolyl)-1H-pyrazol- 1-yl)furo[3,2- d]pyrimidine-6- carboxamide | 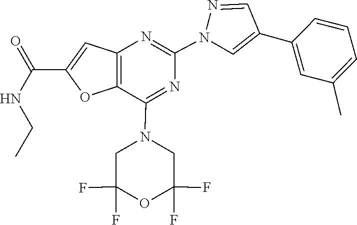 [SMILES: CCNC(=O)c1cc2nc(-n3cc(-c4cccc(C)c4)cn3)nc(N3CC(F)(F)OC(F)(F)C3)c2o1] | 30 | LC-MS (ESI+): m/z 505 (MH⁺). ¹HNMR (300 MHz, CDCl₃) δ 8.69 (s, 1H), 8.10 (s, 1H), 7.51 (s, 1H), 7.44- 7.41 (m, 2H), 7.34-7.29 (m, 1H), 7.13 (d, J = 7.5 Hz, 1H), 6.40-6.38 (m, 1H), 4.63-4.58 (m, 4H), 3.63-3.54 (m, 2H), 2.42 (s, 3H), 1.32 (t, J = 7.2 Hz, 3H). |
| 135 | morpholino(4- morpholino-2-((5- phenyl-1H- pyrazol-3- yl)amino)furo[3,2- d]pyrimidin-6- yl)methanone | 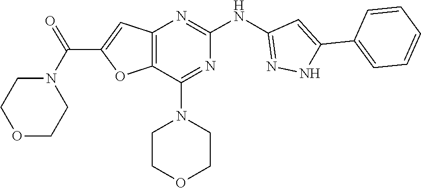 [SMILES: O=C(c1cc2nc(Nc3cc(-c4ccccc4)[nH]n3)nc(N3CCOCC3)c2o1)N1CCOCC1] | 17 | LCMS (M + H)⁺: 476.2; ¹HNMR (300 MHz, DMSO d₆) δ 12.52- 12.38 (1H, broad), 9.66-8.9 (1H, broad), 7.74 (2H, broad), 7.43 (2H, broad), 7.31-6.99 (3H, broad multiplet), 6.2 (1H, broad), 3.8 (8H, dd) and 3.67 (8H, s) ppm. |
| 136 | (4- methylpiperazin-1- yl)(4-morpholino- 2-((5-phenyl-1H- pyrazol-3- yl)amino)furo[3,2- d]pyrimidin-6- yl)methanone | 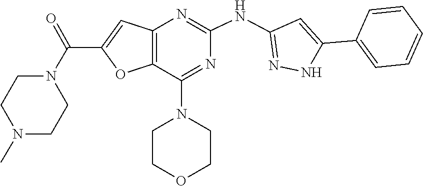 [SMILES: CN1CCN(C(=O)c2cc3nc(Nc4cc(-c5ccccc5)[nH]n4)nc(N4CCOCC4)c3o2)CC1] | 17 | LCMS (M + H)⁺: 489.3; ¹HNMR (300 MHz, DMSO d₆) δ 12.26 (1H, broad), 9.49-8.65 (1H, broad), 7.73-6.95 (7H, multiplet), 6.31 (1H, broad), 3.85 (8H, dd), 2.40 (4H, broad s) and 2.24 (3H, s) ppm. |
| 137 | N,N-dimethyl-4- morpholino-2-((5- phenyl-1H- pyrazol-3- yl)amino)furo[3,2- d]pyrimidine-6- carboxamide | 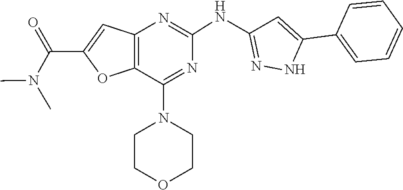 [SMILES: CN(C)C(=O)c1cc2nc(Nc3cc(-c4ccccc4)[nH]n3)nc(N3CCOCC3)c2o1] | 17 | LCMS (M + H)⁺: 434.2; ¹HNMR (300 MHz, DMSO d₆) δ 12.3 (1H, broad), 7.73 (2H, d), 7.42 (2H, t), 7.3 (1H, t), 7.16 (1H, s) and 3.85 (8H, dd) ppm. |
| 138 | N-methyl-4- morpholino-2-((5- phenyl-1H- pyrazol-3- yl)amino)furo[3,2- d]pyrimidine-6- carboxamide | 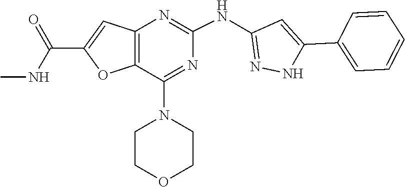 [SMILES: CNC(=O)c1cc2nc(Nc3cc(-c4ccccc4)[nH]n3)nc(N3CCOCC3)c2o1] | 17 | LCMS (M + H)⁺: 420.2; ¹HNMR (300 MHz, DMSO d₆) δ 12.5 (1H, broad), 9.72-9.04 (1H), 8.67 (1H, s), 7.75-7.20 (5H, multiplet), 6.97- 6.27 (1H), 3.85 (8H, dd) and 2.8 (3H, d) ppm. |
| 139 | N-methoxy-N- methyl-4- morpholino-2-((5- phenyl-1H- pyrazol-3- yl)amino)furo[3,2- d]pyrimidine-6- carboxamide | 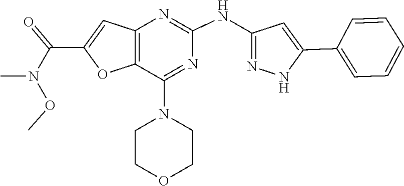 [SMILES: CON(C)C(=O)c1cc2nc(Nc3cc(-c4ccccc4)[nH]n3)nc(N3CCOCC3)c2o1] | 17 | LCMS (M + H)⁺: 450.2; ¹HNMR (300 MHz, CDCl₃) δ 12.33 (1H), 7.75-7.30 (6H, multiplet) 3.85 (8H, dd) 3.80 (3H, s) and 2.90 (s, 3H) ppm. |
| 140 | N-(2- methoxyethyl)-4- morpholino-2-((5- phenyl-1H- pyrazol-3- yl)amino)furo[3,2- d]pyrimidine-6- carboxamide | 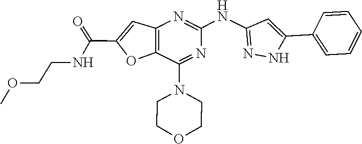 [SMILES: COCCNC(=O)c1cc2nc(Nc3cc(-c4ccccc4)[nH]n3)nc(N3CCOCC3)c2o1] | 17 | LCMS (M + H)⁺: 464.3; ¹HNMR (300 MHz, DMSO d₆) δ 12.29 (1H), 8.51 (1H), 7.75- 7.25 (6H, multiplet), 3.85 (8H, dd), 3.55- 3.50 (4H, multiplet) and 3.47 (3H, s) ppm. |
| 141 | 4-morpholino-2- ((5-phenyl-1H- pyrazol-3- yl)amino)furo[3,2- d]pyrimidine-6- carboxylic acid | 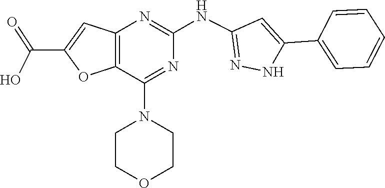 [SMILES: O=C(O)c1cc2nc(Nc3cc(-c4ccccc4)[nH]n3)nc(N3CCOCC3)c2o1] | 17 | LCMS (M + H)⁺: 407.1; ¹HNMR (300 MHz, DMSO d₆) δ 12.5 (1H, broad), 9.3 (1H, broad), 7.72 (2H, d), 7.42 (2H, t), 7.30 (1H, t), 6.77 (1H, s), 6.6 (1H, broad) and 3.84 (8H, dd) ppm. |
| 142 | 4-morpholino-N- (5-phenyl-1H- pyrazol-3- yl)furo[3,2- d]pyrimidin-2- amine | 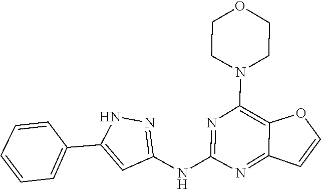 [SMILES: c1ccc(-c2cc(Nc3nc(N4CCOCC4)c4occc4n3)n[nH]2)cc1] | 2 | LCMS (M + H)⁺: 363.1; ¹HNMR (300 MHz, DMSO d₆) δ 11.1 (1H, broad) 8.37 (1H, d), 7.80 (2H, d), 7.49 (2H, t), 7.4 (1H,t), 7.13 (1H), 6.6 (1H), 4.09 (4H, broad), and 3.81 (broad, 4H) ppm. |

### Example 32: Compound 143 Using General Synthetic Route 32

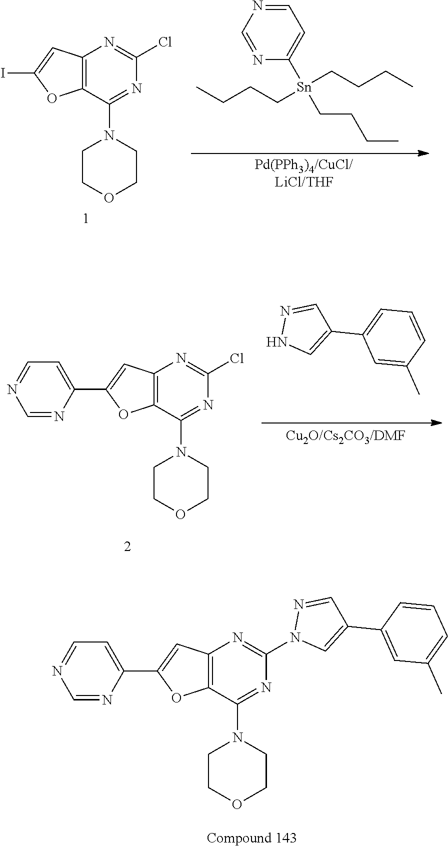

### 1) Synthesis of 2-chloro-4-morpholino-6-(pyrimidin-4-yl)furo[3,2-d]pyrimidine

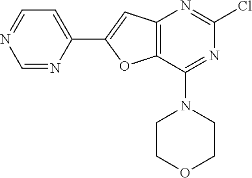 [SMILES: Clc1nc(N2CCOCC2)c2oc(-c3ccncn3)cc2n1]

A suspension of 2-chloro-6-iodo-4-morpholinofuro[3,2-d]pyrimidine (80 mg, 0.22 mmol), 4-(tributylstannyl)pyrimidine (128 mg, 0.34 mmol), LiCl (1 mg, 0.022 mmol) and Pd(PPh₃)₄ (25 mg, 0.022 mmol) in DMF (5 mL) under N₂ was heated to 90° C. for 3 h. The completion of the reaction was monitored by TLC. The reaction mixture was concentrated directly. The resulting residue was purified by silica gel column chromatography with a gradient elution of 1% MeOH/DCM to 2% MeOH/DCM to provide 2-chloro-4-morpholino-6-(pyrimidin-4-yl)furo[3,2-d] pyrimidine (87 mg, 0.27 mmol) as a yellow solid. LC-MS (ESI+): m/z 318/320 (MH⁺).

### 2) Synthesis of 4-morpholino-6-(pyrimidin-4-yl)-2-(4-(m-tolyl)-1H-pyrazol-1-yl)furo[3,2-d]pyrimidine

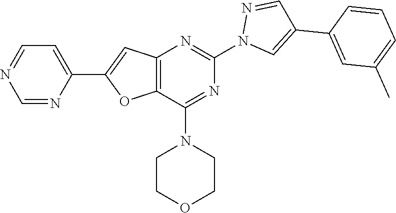 [SMILES: Cc1cccc(-c2cnn(-c3nc(N4CCOCC4)c4oc(-c5ccncn5)cc4n3)c2)c1]

To a solution of 2-chloro-4-morpholino-6-(pyrimidin-4-yl)furo[3,2-d]pyrimidine (73 mg, 0.23 mmol) in DMF (10 mL) was added 4-(m-tolyl)-1H-pyrazole (44 mg, 0.28 mmol), Cs₂CO₃ (151 mg, 0.46 mmol) and Cu₂O (4 mg, 0.023 mmol). The reaction mixture was stirred at 110° C. overnight. The completion of the reaction was monitored by TLC. The reaction mixture was concentrated directly. The resulting residue was purified by silica gel column chromatography with a gradient elution of 2% MeOH/DCM to 10% MeOH/DCM to provide 4-morpholino-6-(pyrimidin-4-yl)-2-(4-(m-tolyl)-1H-pyrazol-1-yl)furo[3,2-d]pyrimidine (Compound 143, 28 mg, 0.064 mmol) as a white solid. LC-MS (ESI+): m/z 440 (MH⁺). ¹HNMR (300 MHz, DMSO-d₆) δ 9.34 (s, 1H), 9.05 (d, J=4.2 Hz, 2H), 8.31-8.14 (m, 2H), 7.87-7.82 (m, 1H), 7.62-7.57 (m, 2H), 7.32-7.27 (m, 1H), 7.10-7.05 (m, 1H), 4.27-4.14 (m, 4H), 3.95-3.85 (m, 4H), 2.36 (s, 3H).

### Example 33: Compound 156 Using General Synthetic Route 33

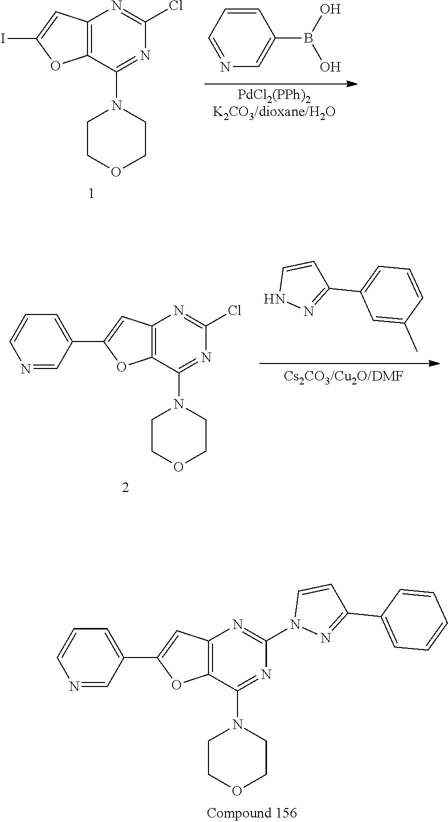

### 1) Synthesis of 2-chloro-4-morpholino-6-(pyridin-3-yl)furo[3,2-d]pyrimidine

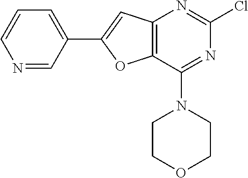 [SMILES: Clc1nc(N2CCOCC2)c2oc(-c3cccnc3)cc2n1]

To a solution of 2-chloro-6-iodo-4-morpholinofuro[3,2-d]pyrimidine (500 mg, 1.37 mmol) in 1,4-dioxane/H₂O (2/1, 30 mL) was added pyridin-3-ylboronic acid (185 mg, 1.51 mmol), K₂CO₃ (378 mg, 2.74 mmol) and PdCl₂(PPh₃)₂ (48 mg, 0.068 mmol) under N₂. The reaction mixture was stirred at 90° C. for 5 h. The completion of the reaction was monitored by TLC. The solution was concentrated directly and the resulting residue was purified by silica gel column chromatography with a gradient elution of 1% MeOH/DCM to 3% MeOH/DCM to provide 2-chloro-4-morpholino-6-(pyridin-3-yl)furo[3,2-d]pyrimidine (410 mg, 1.29 mmol) as a light yellow solid. LC-MS (ESI+): m/z 317/319 (MH⁺). ¹HNMR (300 MHz, CDCl₃) δ 9.07 (s, 1H), 8.69 (d, J=6.9 Hz, 1H), 8.05 (d, J=8.1 Hz, 1H), 7.71-7.40 (m, 1H), 7.08 (s, 1H), 4.15-4.08 (m, 4H), 3.92-3.86 (m, 4H).

### 2) Synthesis of 4-morpholino-2-(3-phenyl-1H-pyrazol-1-yl)-6-(pyridin-3-yl)furo[3,2-d]pyrimidine

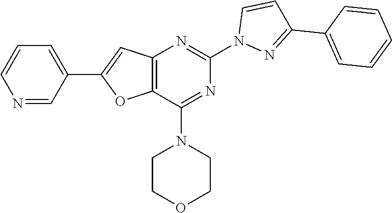 [SMILES: c1ccc(-c2ccn(-c3nc(N4CCOCC4)c4oc(-c5cccnc5)cc4n3)n2)cc1]

To a solution of 2-chloro-4-morpholino-6-(pyridin-3-yl)furo[3,2-d]pyrimidine (120 mg, 0.38 mmol) in DMF (10 mL) was added 3-phenyl-1H-pyrazole (60 mg, 0.42 mmol), Cs₂CO₃ (248 mg, 0.76 mmol) and Cu₂O (6 mg, 0.038 mmol). The reaction was stirred at 110° C. overnight. The completion was monitored by TLC. The reaction mixture was quenched with water (50 mL). The aqueous solution was extracted with DCM (3×30 mL). The combined organic phase was dried over anhydrous Na₂SO₄, filtrated and concentrated under reduce pressure. The resulting residue was purified by silica gel column chromatography with a gradient elution of 1% MeOH/DCM to 3% MeOH/DCM to provide 4-morpholino-2-(3-phenyl-1H-pyrazol-1-yl)-6-(pyridin-3-yl)furo[3,2-d]pyrimidine (114 mg, 0.27 mmol) as an off-white solid. LC-MS (ESI+): m/z 425 (MH⁺). ¹HNMR (300 MHz, CDCl₃) δ 9.11 (s, 1H), 8.69 (d, J=3.9 Hz, 1H), 8.57 (d, J=2.7 Hz, 1H), 8.09 (d, J=8.1 Hz, 1H), 8.01 (d, J=6.9 Hz, 2H), 7.45-7.32 (m, 4H), 7.30-7.26 (m, 1H), 6.79 (d, J=2.7 Hz, 1H), 4.20-4.14 (m, 4H), 3.97-3.92 (m, 4H).

### Example 34: Compounds 145 and 146 Using General Synthetic Route 34

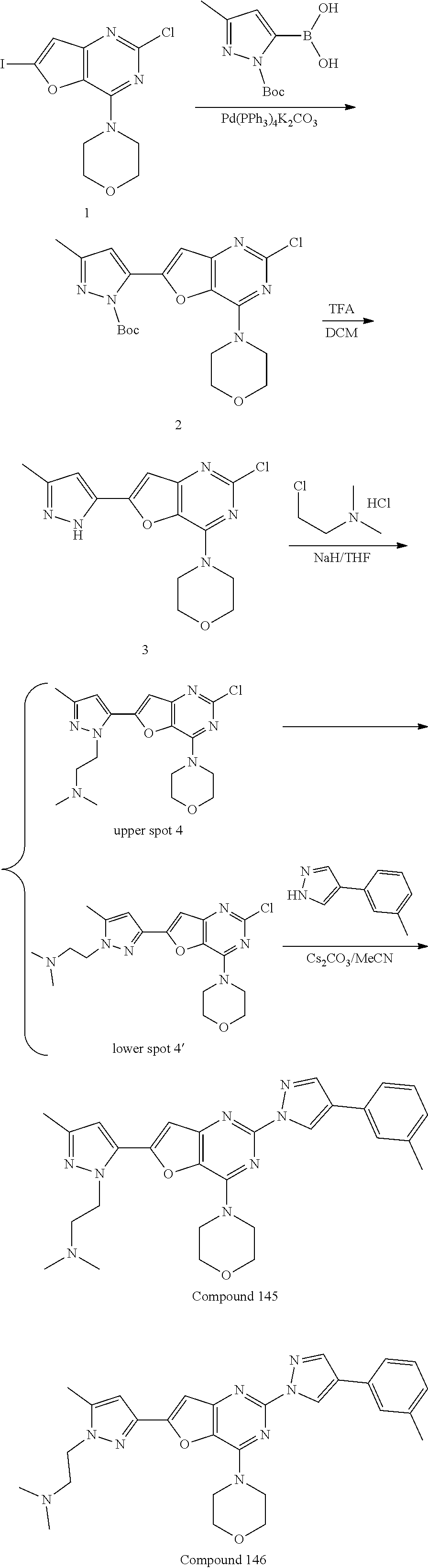

### 1) Synthesis of tert-butyl 5-(2-chloro-4-morpholinofuro[3,2-d]pyrimidin-6-yl)-3-methyl-1H-pyrazole-1-carboxylate

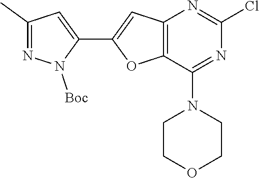 [SMILES: Cc1cc(-c2cc3nc(Cl)nc(N4CCOCC4)c3o2)n(C(=O)OC(C)(C)C)n1]

A suspension of 2-chloro-6-iodo-4-morpholinofuro[3,2-d]pyrimidine (5.72 g, 15.67 mmol), (1-(tert-butoxycarbonyl)-3-methyl-1H-pyrazol-5-yl)boronic acid (3.90 g, 17.24 mmol), Pd(PPh)₂Cl₂ (2.20 g, 3.13 mmol) and CsF (7.15 g, 47.01 mmol) in 1,4-dioxane/H₂O (4/1, 330 mL) under N₂ was heated to 80° C. for 1 h. The completion of the reaction was monitored by TLC. The reaction mixture was concentrated directly and the resulting residue was purified by silica gel column chromatography with a gradient elution of 25% EtOAc/Hex to EtOAc to provide tert-butyl 5-(2-chloro-4-morpholinofuro[3,2-d]pyrimidin-6-yl)-3-methyl-1H-pyrazole-1-carboxylate (10.32 g, 24.57 mmol) as a light yellow solid. LC-MS (ESI+): m/z 420/422 (MH⁺). ¹HNMR (300 MHz, DMSO-d₆) δ 7.31 (s, 1H), 6.94 (s, 1H), 4.01-3.90 (m, 4H), 3.78-3.70 (m, 4H), 2.21 (s, 3H), 1.44 (s, 9H).

### 2) Synthesis of 2-chloro-6-(3-methyl-1H-pyrazol-5-yl)-4-morpholinofuro[3,2-d]pyrimidine

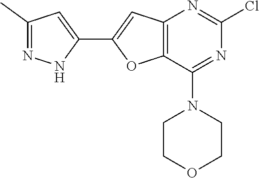 [SMILES: Cc1cc(-c2cc3nc(Cl)nc(N4CCOCC4)c3o2)[nH]n1]

To a solution of tert-butyl 5-(2-chloro-4-morpholinofuro[3,2-d]pyrimidin-6-yl)-3-methyl-1H-pyrazole-1-carboxylate (10.32 g, 24.63 mmol) in DCM (300 mL) was added TFA (30 mL). The mixture was stirred at rt for 2 h. The completion of the reaction was monitored by TLC. The reaction mixture was quenched with saturated NaHCO₃ solution until the pH=8. A large amount of solid was precipitated. After filtration, the filter cake was washed with ether twice to provide crude 4-(5-(benzyloxy)-2-(5-methyl-1H-pyrazol-3-yl)pyrazolo[1,5-a]pyrimidin-7-yl)morpholine (7.96 g, 24.95 mmol) as a light yellow solid. The crude product was used directly for the next step without further purification. LC-MS (ESI+): m/z 320/322 (MH⁺).

### 3) Synthesis of 2-(5-(2-chloro-4-morpholinofuro[3,2-d]pyrimidin-6-yl)-3-methyl-1H-pyrazol-1-yl)-N,N-dimethylethan-1-amine (to Compound 146) and 2-(3-(2-chloro-4-morpholinofuro[3,2-d]pyrimidin-6-yl)-5-methyl-1H-pyrazol-1-yl)-N,N-dimethylethan-1-amine (to Compound 145)

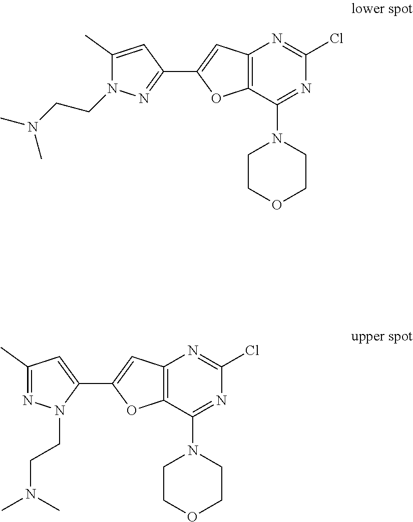 [SMILES: Cc1cc(-c2cc3nc(Cl)nc(N4CCOCC4)c3o2)nn1CCN(C)C ; Cc1cc(-c2cc3nc(Cl)nc(N4CCOCC4)c3o2)n(CCN(C)C)n1]

To a solution of crude 4-(5-(benzyloxy)-2-(5-methyl-1H-pyrazol-3-yl)pyrazolo [1,5-a]pyrimidin-7-yl)morpholine (170 mg, 0.53 mmol) in DMF was added K₂CO₃ (220 mg, 1.60 mmol) and 2-chloro-N,N-dimethylethanamine hydrochloride (115 mg, 0.80 mmol). The mixture was stirred at 50° C. for 2 h. The completion of the reaction was monitored by TLC. The reaction mixture was quenched with water and extracted with EtOAc (3×10 mL). The combined organic phase was dried over anhydrous Na₂SO₄, filtrated and concentrated under reduce pressure. The residue was purified by preparative TLC with an elution of 10% MeOH/DCM to provide 2-(3-(2-chloro-4-morpholinofuro[3,2-d]pyrimidin-6-yl)-5-methyl-1H-pyrazol-1-yl)-N,N-dimethylethanamine (lower spot, 90 mg, 0.23 mmol) and 2-(5-(2-chloro-4-morpholinofuro[3,2-d]pyrimidin-6-yl)-3-methyl-1H-pyrazol-1-yl)-N,N-dimethylethanamine (upper spot, 60 mg, 0.15 mmol).

2-(3-(2-chloro-4-morpholinofuro[3,2-d]pyrimidin-6-yl)-5-methyl-1H-pyrazol-1-yl)-N,N-dimethylethanamine (lower spot): LC-MS (ESI+): m/z 391/393 (MH⁺) ¹HNMR (300 MHz, DMSO-d₆) δ 7.06 (s, 1H), 6.65 (s, 1H), 4.23 (t, J=6.3 Hz, 2H), 3.96-3.88 (m, 4H), 3.82-3.75 (m, 4H), 2.71 (t, J=6.0 Hz, 2H), 2.35 (s, 3H), 2.24 (s, 6H). 2-(5-(2-chloro-4-morpholinofuro[3,2-d]pyrimidin-6-yl)-3-methyl-1H-pyrazol-1-yl)-N,N-dimethylethanamine (upper spot): LC-MS (ESI+): m/z 391/393 (MH⁺) ¹HNMR (300 MHz, DMSO-d₆) δ 7.32 (s, 1H), 6.73 (s, 1H), 4.43 (t, J=6.9 Hz, 2H), 3.97-3.88 (m, 4H), 3.82-3.73 (m, 4H), 2.68 (t, J=6.9 Hz, 2H), 2.21 (s, 3H), 2.18 (s, 6H).

### 4) Synthesis of N,N-dimethyl-2-(5-methyl-3-(4-morpholino-2-(4-(m-tolyl)-1H-pyrazol-1-yl)furo[3,2-d]pyrimidin-6-yl)-1H-pyrazol-1-yl)ethanamine (Compound 146)

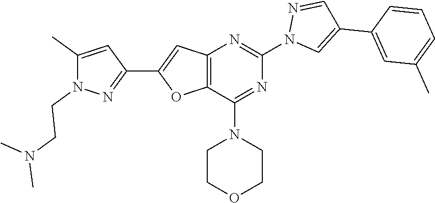 [SMILES: Cc1cccc(-c2cnn(-c3nc(N4CCOCC4)c4oc(-c5cc(C)n(CCN(C)C)n5)cc4n3)c2)c1]

A suspension of 2-(3-(2-chloro-4-morpholinofuro[3,2-d]pyrimidin-6-yl)-5-methyl-1H-pyrazol-1-yl)-N,N-dimethylethanamine (50 mg, 0.13 mmol), 4-(m-tolyl)-1H-pyrazole (24 mg, 0.15 mmol), Cs₂CO₃ (83 mg, 0.26 mmol) and Cu₂O (1.8 mg, 0.01 mmol) in DMF (5 mL) was heated to 110° C. overnight. The completion of the reaction was monitored by TLC. The reaction mixture was concentrated directly and purified by silica gel column chromatography with a gradient elution of 5% MeOH/DCM to 10% MeOH/DCM to provide N,N-dimethyl-2-(5-methyl-3-(4-morpholino-2-(4-(m-tolyl)-1H-pyrazol-1-yl)furo[3,2-d]pyrimidin-6-yl)-1H-pyrazol-1-yl)ethanamine (20 mg, 0.04 mmol) as a white solid. LC-MS (ESI+): m/z 513 (MH⁺). ¹HNMR (300 MHz, CDCl₃) δ 8.72 (s, 1H), 8.07 (s, 1H), 7.61-7.40 (m, 2H), 7.35-7.27 (m, 1H), 7.11-7.06 (m, 2H), 6.44 (s, 1H), 4.40-4.37 (m, 2H), 4.16-4.10 (m, 4H), 3.93-3.88 (m, 4H), 3.10-2.98 (m, 2H), 2.45 (m, 3H), 2.41 (s, 9H).

### 5) Synthesis of N,N-dimethyl-2-[3-methyl-5-[4-morpholino-2-[4-(m-tolyl)pyrazol-1-yl]furo[3,2-d]pyrimidin-6-yl]pyrazol-1-yl]ethanamine (Compound 145)

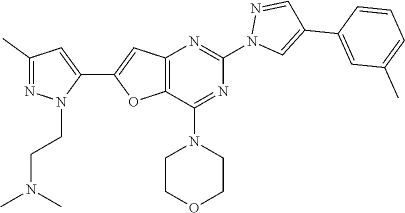 [SMILES: Cc1cccc(-c2cnn(-c3nc(N4CCOCC4)c4oc(-c5cc(C)nn5CCN(C)C)cc4n3)c2)c1]

Compound 145 was prepared by the same method used for Compound 146.

### Example 35: Compound 148 Using General Synthetic Route 35

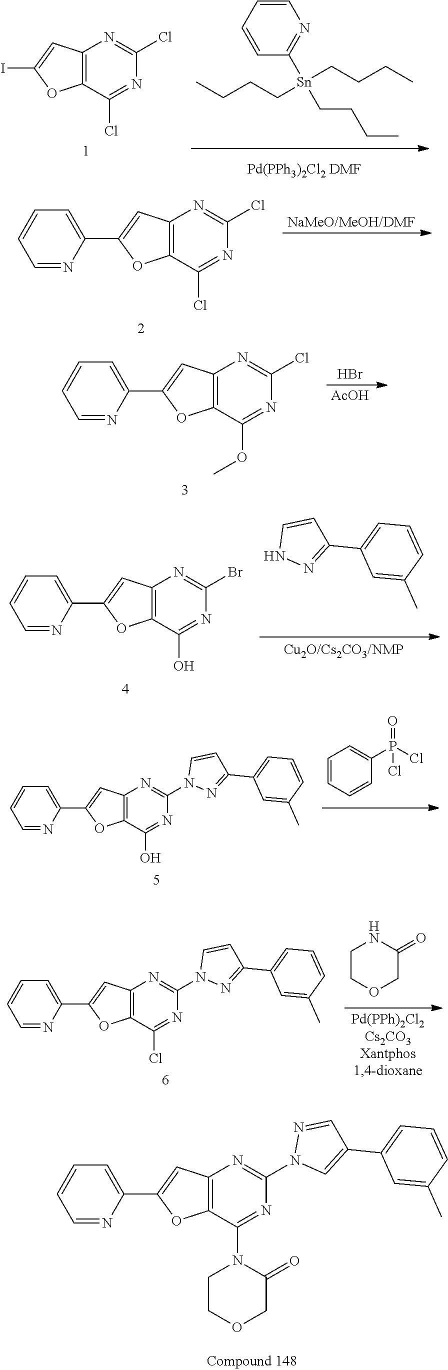

### 1) Synthesis of 2,4-dichloro-6-(pyridin-2-yl)furo[3,2-d]pyrimidine

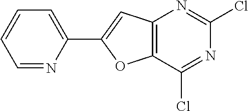 [SMILES: Clc1nc(Cl)c2oc(-c3ccccn3)cc2n1]

To a solution of 2,4-dichloro-6-iodofuro[3,2-d]pyrimidine (2.3 g, 7.3 mmol) in DMF (60 mL) under N₂ was added 2-(tributylstannyl)pyridine (2.7 g, 7.3 mmol), CuI (416 mg, 2.2 mmol) and PdCl₂(dppf) (534 mg, 0.73 mmol). The reaction was stirred at 100° C. for 3 h. The completion of the reaction was monitored by TLC. The reaction mixture was quenched with water (30 mL) and extracted with DCM (4×20 mL). The combined organic phase was dried over anhydrous Na₂SO₄, filtrated and concentrated under reduce pressure. The resulting residue was purified by silica gel column chromatography with a gradient elution of 10% EtOAc/PE to 30% EtOAc/PE to provide 2,4-dichloro-6-(pyridin-2-yl)furo[3,2-d]pyrimidine (1.2 g, 4.53 mmol) as a yellow solid. LC-MS (ESI+): m/z 266/268 (MH⁺). ¹HNMR (300 MHz, CDCl₃) δ 8.78 (d, J=4.2 Hz, 1H), 8.09 (d, J=7.8 Hz, 1H), 7.92 (t, J=7.8 Hz, 1H), 7.56 (s, 1H), 7.47-7.41 (m, 1H).

### 2) Synthesis of 2-chloro-4-methoxy-6-(pyridin-2-yl)furo[3,2-d]pyrimidine

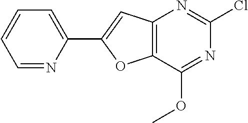 [SMILES: COc1nc(Cl)nc2cc(-c3ccccn3)oc12]

To a solution of 2,4-dichloro-6-(pyridin-2-yl)furo[3,2-d]pyrimidine (500 mg, 1.89 mmol) in DMF/MeOH (1:1, 30 mL) at 0° C. was added sodium methanolate (204 mg, 3.78 mmol). The reaction was stirred at 0° C. for 3 h. The completion of the reaction was monitored by TLC. The reaction mixture was quenched with water (30 mL) and a large amount of solid was precipitated. After filtration, the filter cake was washed with Et₂O to provide 2-chloro-4-methoxy-6-(pyridin-2-yl)furo[3,2-d]pyrimidine (450 mg, 1.72 mmol) as a brown solid. LC-MS (ESI+): m/z 262/264 (MH⁺). ¹HNMR (300 MHz, CDCl₃) ¹HNMR (300 MHz, CDCl₃) δ 8.78 (d, J=4.2 Hz, 1H), 8.09 (d, J=7.8 Hz, 1H), 7.85 (t, J=7.8 Hz, 1H), 7.53 (s, 1H), 7.39-7.35 (m, 1H), 4.23 (s, 3H).

### 3) Synthesis of 2-bromo-6-(pyridin-2-yl)furo[3,2-d]pyrimidin-4-ol

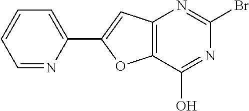 [SMILES: Oc1nc(Br)nc2cc(-c3ccccn3)oc12]

A solution of 2-chloro-4-methoxy-6-(pyridin-2-yl)furo[3,2-d]pyrimidine (200 mg, 0.76 mmol) in HBr/AcOH (33 wt. % in Acetic acid, 10 mL) was heated to refluxed for 2 h. The completion of the reaction was monitored by LC-MS. The reaction mixture was quenched with water (30 mL) and a large amount of solid was precipitated. After filtration, the filter cake was washed with Et₂O to provide 292 mg of crude 2-bromo-6-(pyridin-2-yl)furo[3,2-d]pyrimidin-4-ol as a yellow solid. The crude product was used directly for the next step without further purification. LC-MS (ESI+): m/z 292/294 (MH⁺).

### 4) Synthesis of 6-(pyridin-2-yl)-2-(3-(m-tolyl)-1H-pyrazol-1-yl)furo[3,2-d]pyrimidin-4-ol

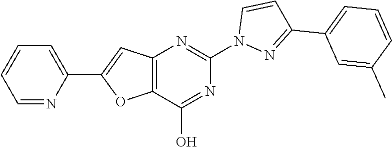 [SMILES: Cc1cccc(-c2ccn(-c3nc(O)c4oc(-c5ccccn5)cc4n3)n2)c1]

A suspension of crude 2-bromo-6-(pyridin-2-yl)furo[3,2-d]pyrimidin-4-ol (200 mg, 0.68 mmol), 3-(m-tolyl)-1H-pyrazole (108 mg, 0.68 mmol), Cs₂CO₃ (447 mg, 1.37 mmol) and Cu₂O (10 mg, 0.068 mmol) in DMF (10 mL) was heated to 110° C. overnight. The completion of the reaction was monitored by TLC. The reaction mixture was concentrated directly and purified by silica gel column chromatography with a gradient elution of 5% MeOH/DCM to 10% MeOH/DCM to provide 6-(pyridin-2-yl)-2-(3-(m-tolyl)-1H-pyrazol-1-yl)furo[3,2-d]pyrimidin-4-ol (100 mg, 0.27 mmol) as a green solid. LC-MS (ESI+): m/z 370 (MH⁺).

### 5) Synthesis of 4-chloro-6-(pyridin-2-yl)-2-(3-(m-tolyl)-1H-pyrazol-1-yl)furo[3,2-d]pyrimidine

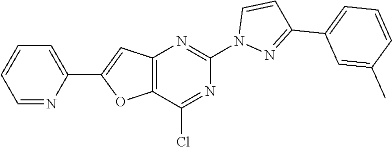 [SMILES: Cc1cccc(-c2ccn(-c3nc(Cl)c4oc(-c5ccccn5)cc4n3)n2)c1]

A solution of 6-(pyridin-2-yl)-2-(3-(m-tolyl)-1H-pyrazol-1-yl)furo[3,2-d]pyrimidin-4-ol (114 mg, 0.31 mmol) in phenylphosphonic dichloride (5 mL) was heated to 120° C. for 2 h. The completion of the reaction was monitored by TLC. The reaction mixture was quenched with water (20 mL) and a large amount of yellow solid was precipitated. After filtration, the filter cake was washed with Et₂O to provide 80 mg of crude 4-chloro-6-(pyridin-2-yl)-2-(3-(m-tolyl)-1H-pyrazol-1-yl)furo[3,2-d]pyrimidine as a yellow solid. The crude product was used directly for the next step without further purification. LC-MS (ESI+): m/z 388/390 (MH⁺).

### 6) Synthesis of 4-(6-(pyridin-2-yl)-2-(4-(m-tolyl)-1H-pyrazol-1-yl)furo[3,2-d]pyrimidin-4-yl)morpholin-3-one

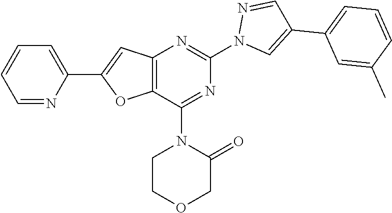 [SMILES: Cc1cccc(-c2cnn(-c3nc(N4CCOCC4=O)c4oc(-c5ccccn5)cc4n3)c2)c1]

A suspension of 4-chloro-6-(pyridin-2-yl)-2-(3-(m-tolyl)-1H-pyrazol-1-yl)furo[3,2-d]pyrimidine (40 mg, 0.1 mmol), morpholin-3-one (12.5 mg, 0.12 mmol), Pd(PPh)₂Cl₂ (7.2 mg, 0.01 mmol), Cs₂CO₃ (78 mg, 0.2 mmol) and Xantphos (12 mg, 0.02 mmol) in 1,4-dioxane (10 mL) under N₂ was heated to 90° C. for 1 h. The completion of the reaction was monitored by TLC. The reaction mixture was quenched with water and extracted with EtOAc (3×10 mL). The combined organic phase was dried over anhydrous Na₂SO₄, filtrated and concentrated under reduced pressure. The resulting residue was purified by preparative TLC with a elution of 10% MeOH/DCM to provide 4-(6-(pyridin-2-yl)-2-(4-(m-tolyl)-1H-pyrazol-1-yl)furo[3,2-d]pyrimidin-4-yl)morpholin-3-one (5 mg, 0.011 mmol) as a white solid. LC-MS (ESI+): m/z 453 (MH⁺). ¹HNMR (300 MHz, CDCl₃) δ 8.75 (d, J=3.9 Hz, 1H), 8.62 (s, 1H), 8.02 (d, J=7.8 Hz, 1H), 7.96-7.89 (m, 2H), 7.76 (d, J=7.5 Hz, 1H), 7.67 (s, 1H), 7.48-7.31 (m, 2H), 7.20-7.15 (m, 1H), 6.82 (s, 1H), 4.53 (s, 2H), 4.32-4.24 (m, 2H), 4.19-4.10 (m, 2H), 2.43 (s, 3H).

### Example 36: Compound 150 Using General Synthetic Route 36

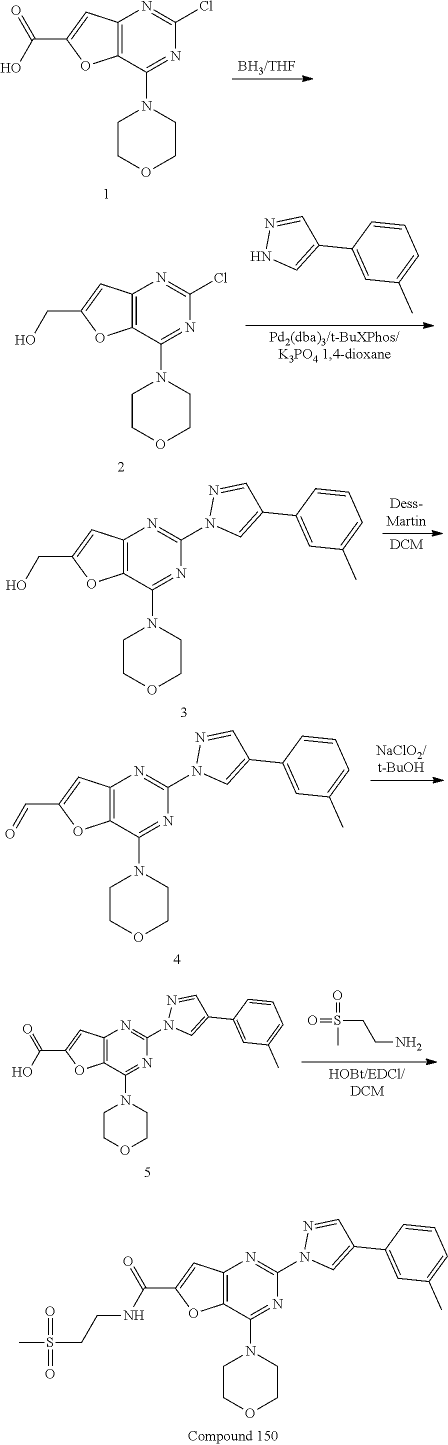

### 1) Synthesis of (2-chloro-4-morpholinofuro[3,2-d]pyrimidin-6-yl)methanol

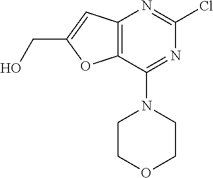 [SMILES: OCc1cc2nc(Cl)nc(N3CCOCC3)c2o1]

To a solution of 2-chloro-4-morpholinofuro[3,2-d]pyrimidine-6-carboxylic acid (1 g, 3.53 mmol) in THF (10 mL) at 0° C. was added BH₃/THF (1 mol/L, 14 mL) dropwise. The reaction mixture was stirred at rt overnight. The completion of the reaction was monitored by TLC. The reaction was quenched with 1N HCl. The mixture was heated under reflux for 2 h. The reaction mixture was concentrated directly and purified by silica gel column chromatography with a gradient elution of 1% MeOH/DCM to 3% MeOH/DCM to provide (2-chloro-4-morpholinofuro[3,2-d]pyrimidin-6-yl)methanol (165 mg, 0.61 mmol) as a white solid. LC-MS (ESI+): m/z 270 (MH⁺).

### 2) Synthesis of (4-morpholino-2-(4-(m-tolyl)-1H-pyrazol-1-yl)furo[3,2-d]pyrimidin-6-yl)methanol

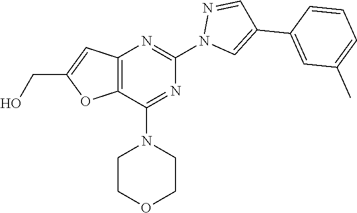 [SMILES: Cc1cccc(-c2cnn(-c3nc(N4CCOCC4)c4oc(CO)cc4n3)c2)c1]

To a solution of (2-chloro-4-morpholinofuro[3,2-d]pyrimidin-6-yl)methanol (250 mg, 0.93 mmol) in 1,4-dioxane (20 mL) was added 4-(m-tolyl)-1H-pyrazole (176 mg, 0.42 mmol), Pd₂(dba)₃ (85 mg, 0.093 mmol), t-Buxphos and K₃PO₄ (900 mg, 3.72 mmol). The reaction mixture was stirred at 90° C. overnight. The completion of the reaction was monitored by TLC. The reaction mixture was quenched with water (50 mL). The aqueous solution was extracted with DCM (3×30 mL). The combined organic phase was dried over anhydrous Na₂SO₄, filtrated and concentrated under reduce pressure. The resulting residue was purified by silica gel column chromatography with a gradient elution of 3% MeOH/DCM to 8% MeOH/DCM to provide (4-morpholino-2-(4-(m-tolyl)-1H-pyrazol-1-yl)furo[3,2-d]pyrimidin-6-yl)methanol (120 mg, 0.31 mmol) as an off-white solid. LC-MS (ESI+): m/z 392 (MH⁺).

### 3) Synthesis of 4-morpholino-2-(4-(m-tolyl)-1H-pyrazol-1-yl)furo[3,2-d]pyrimidine-6-carbaldehyde

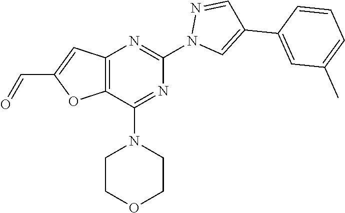 [SMILES: Cc1cccc(-c2cnn(-c3nc(N4CCOCC4)c4oc(C=O)cc4n3)c2)c1]

To a solution of (4-morpholino-2-(4-(m-tolyl)-1H-pyrazol-1-yl)furo[3,2-d]pyrimidin-6-yl) methanol (60 mg, 0.15 mmol) in DCM at rt (10 mL) was added Dess-Martin periodinane (DMP) (120 mg, 0.28 mmol). The reaction was stirred at rt for 2 h. The completion of the reaction was monitored by TLC. The reaction was quenched with saturated NaHCO₃ solution. The aqueous solution was extracted with MeOH/DCM (1/10, 3×10 mL). The combined organic phase was dried over anhydrous Na₂SO₄, filtrated and concentrated under reduce pressure. The residue was purified by silica gel column chromatography with a gradient elution of 1% MeOH/DCM to 3% MeOH/DCM to provide 4-morpholino-2-(4-(m-tolyl)-1H-pyrazol-1-yl)furo[3,2-d]pyrimidine-6-carbaldehyde (32 mg, 0.08 mmol) as a light yellow solid. LC-MS (ESI+): m/z 390 (MH⁺). ¹HNMR (300 MHz, CDCl₃) δ 9.94 (s, 1H), 8.69 (s, 1H), 8.09 (s, 1H), 7.61 (s, 1H), 7.48-7.40 (m, 2H), 7.35-7.26 (m, 2H), 7.11 (d, J=7.8 Hz, 1H), 4.25-4.15 (m, 4H), 3.95-3.85 (m, 4H), 2.41 (s, 3H).

### 4) Synthesis of 4-morpholino-2-(4-(m-tolyl)-1H-pyrazol-1-yl)furo[3,2-d]pyrimidine-6-carboxylic acid

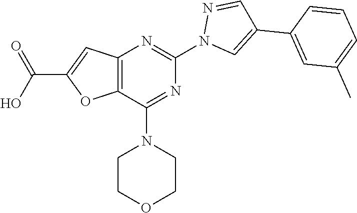 [SMILES: Cc1cccc(-c2cnn(-c3nc(N4CCOCC4)c4oc(C(=O)O)cc4n3)c2)c1]

To a solution of 4-morpholino-2-(4-(m-tolyl)-1H-pyrazol-1-yl)furo[3,2-d]pyrimidine-6-carbaldehyde (32 mg, 0.08 mmol) in i-PrOH/H₂O (4:1, 5 mL) at 0° C. was added NaH₂PO₄.H₂O (64.2 mg, 0.41 mmol) and NaClO₂ (37 mg, 0.41 mmol) in portions. The reaction was stirred at rt for 1 h. The completion of the reaction was monitored by TLC. The reaction was quenched with a 1N HCl solution. The aqueous solution was extracted with MeOH/DCM (1/10, 3×10 mL). The combined organic phase was dried over anhydrous Na₂SO₄, filtrated and concentrated to provide crude 4-morpholino-2-(4-(m-tolyl)-1H-pyrazol-1-yl)furo[3,2-d]pyrimidine-6-carboxylic acid (38 mg, 0.09 mmol) as a white solid. LC-MS (ESI+): m/z 406 (MH⁺)

### 5) Synthesis of N-(2-(methylsulfonyl)ethyl)-4-morpholino-2-(4-(m-tolyl)-1H-pyrazol-1-yl)furo[3,2-d]pyrimidine-6-carboxamide

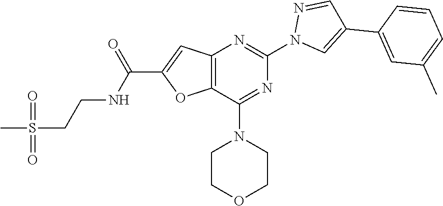 [SMILES: Cc1cccc(-c2cnn(-c3nc(N4CCOCC4)c4oc(C(=O)NCCS(C)(=O)=O)cc4n3)c2)c1]

A solution of 4-morpholino-2-(4-(m-tolyl)-1H-pyrazol-1-yl)furo[3,2-d]pyrimidine-6-carboxylic acid (38 mg, 0.09 mmol), 2-(methylsulfonyl)ethanamine (16 mg, 0.10 mmol), EDCl (37.8 mg, 0.20 mmol) and HOBT (26 mg, 0.20 mmol) in DCM was stirred at rt for 2 h. The completion of the reaction was monitored by TLC. The reaction mixture was concentrated directly and purified by silica gel column chromatography with a gradient elution of 2% MeOH/DCM to 5% MeOH/DCM to provide N-(2-(methylsulfonyl)ethyl)-4-morpholino-2-(4-(m-tolyl)-1H-pyrazol-1-yl) furo[3,2-d]pyrimidine-6-carboxamide (14 mg, 0.027 mmol) as a white solid. LC-MS (ESI+): m/z 511 (MH⁺). ¹HNMR (300 MHz, CDCl₃) δ 8.70 (s, 1H), 8.07 (s, 1H), 7.67-7.62 (m, 1H), 7.53 (s, 1H), 7.48-7.40 (m, 2H), 7.35-7.26 (m, 1H), 7.12-7.09 (m, 1H), 4.15-4.05 (m, 6H), 3.92-3.85 (m, 4H), 3.34-3.07 (m, 2H), 3.04 (s, 3H), 2.42 (s, 3H).

### Example 37: Compound 192 Using General Synthetic Route 37

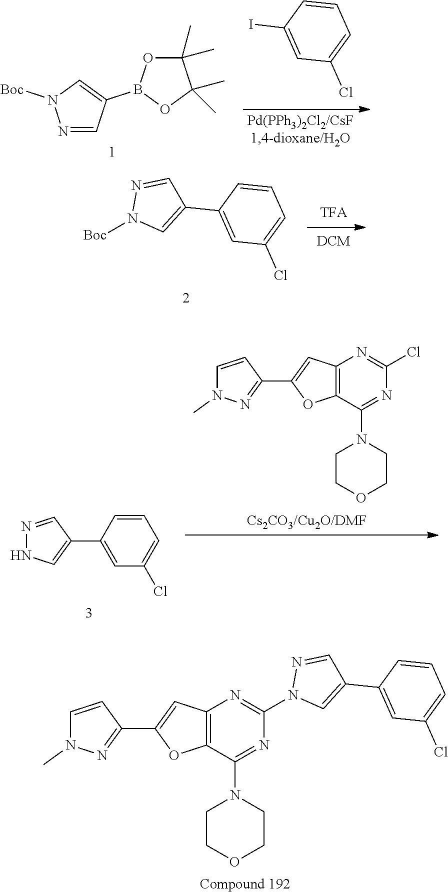

### 1) Synthesis of tert-butyl 4-(3-chlorophenyl)-1H-pyrazole-1-carboxylate

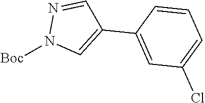 [SMILES: CC(C)(C)OC(=O)n1cc(-c2cccc(Cl)c2)cn1]

To a solution of tert-butyl 4-(4,4,5,5-tetramethyl-1,3,2-dioxaborolan-2-yl)-1H-pyrazole-1-carboxylate (1 g, 4.20 mmol) in 1,4-dioxane/H₂O (10/1, 20 mL) was added 1-chloro-3-iodobenzene (1.24 g, 1.51 mmol), CsF (958 mg, 6.30 mmol) and PdCl₂(PPh₃)₂ (295 mg, 0.42 mmol) under N₂. The reaction was stirred at 80° C. for 2 h. The completion of the reaction was monitored by TLC. The reaction was quenched with water. The aqueous solution was extracted with DCM (3×80 mL). The combined organic phase was dried over anhydrous Na₂SO₄, filtrated and concentrated under reduce pressure. The residue was purified by silica gel column chromatography with a gradient elution of 10% EtOAc/PE to 30% EtOAc/PE to provide tert-butyl 4-(3-chlorophenyl)-1H-pyrazole-1-carboxylate (650 mg, 2.34 mmol) as a light yellow oil. LC-MS (ESI+): m/z 279/281 (MH⁺).

### 2) Synthesis of 4-(3-chlorophenyl)-1H-pyrazole

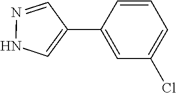 [SMILES: Clc1cccc(-c2cn[nH]c2)c1]

To a solution of tert-butyl 4-(3-chlorophenyl)-1H-pyrazole-1-carboxylate (650 mg, 2.34 mmol) in DCM (10 mL) was added TFA (2 mL). The mixture was stirred at rt for 2 h. The completion of the reaction was monitored by TLC. The reaction mixture was quenched with saturated NaHCO₃ solution until the pH=8. The aqueous solution was extracted with DCM (3×80 mL). The combined organic phase was dried over anhydrous Na₂SO₄, filtrated and concentrated under reduce pressure. The residue was purified by silica gel column chromatography with a gradient elution of 20% EtOAc/PE to 50% EtOAc/PE to provide 4-(3-chlorophenyl)-1H-pyrazole (130 mg, 0.73 mmol) as a light yellow solid. LC-MS (ESI+): m/z 179/181 (MH⁺). ¹HNMR (300 MHz, CDCl₃) δ 7.87 (brs, 2H), 7.46 (s, 1H), 7.39 (d, J=7.8 Hz, 1H), 7.30-7.28 (m, 1H), 7.23-7.20 (m, 1H).

### 3) Synthesis of 2-(4-(3-chlorophenyl)-1H-pyrazol-1-yl)-6-(1-methyl-1H-pyrazol-3-yl)-4-morpholinofuro[3,2-d]pyrimidine

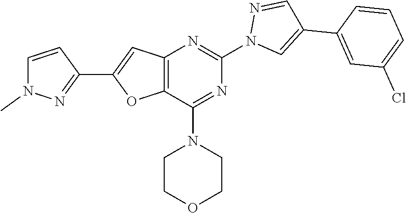 [SMILES: Cn1ccc(-c2cc3nc(-n4cc(-c5cccc(Cl)c5)cn4)nc(N4CCOCC4)c3o2)n1]

To a solution of 2-chloro-6-(1-methyl-1H-pyrazol-3-yl)-4-morpholinofuro[3,2-d]pyrimidine (60 mg, 0.19 mmol) in DMF (10 mL) was added 4-(3-chlorophenyl)-1H-pyrazole (40 mg, 0.19 mmol), Cs₂CO₃ (221 mg, 0.68 mmol) and Cu₂O (5 mg, 0.04 mmol). The reaction was stirred at 110° C. overnight. The completion of the reaction was monitored by TLC. The reaction mixture was quenched with water (50 mL). The aqueous solution was extracted with DCM (3×30 mL). The combined organic phase was dried over anhydrous Na₂SO₄, filtrated and concentrated under reduce pressure. The resulting residue was purified by silica gel column chromatography with a gradient elution of 1% MeOH/DCM to 3% MeOH/DCM to provide 2-(4-(3-chlorophenyl)-1H-pyrazol-1-yl)-6-(1-methyl-1H-pyrazol-3-yl)-4-morpholinofuro[3,2-d]pyrimidine (22 mg, 0.048 mmol) as an off-white solid. LC-MS (ESI+): m/z 462/464 (MH⁺). ¹HNMR (300 MHz, CDCl₃) δ 8.75 (s, 1H), 8.06 (s, 1H), 7.62 (s, 1H), 7.50-7.40 (m, 2H), 7.26-7.20 (m, 2H), 7.11 (s, 1H), 6.66 (d, J=2.4 Hz, 1H), 4.21-4.15 (m, 4H), 4.02 (s, 3H), 3.95-3.89 (m, 4H).

### Example 38: Compound 157 Using General Synthetic Route 38

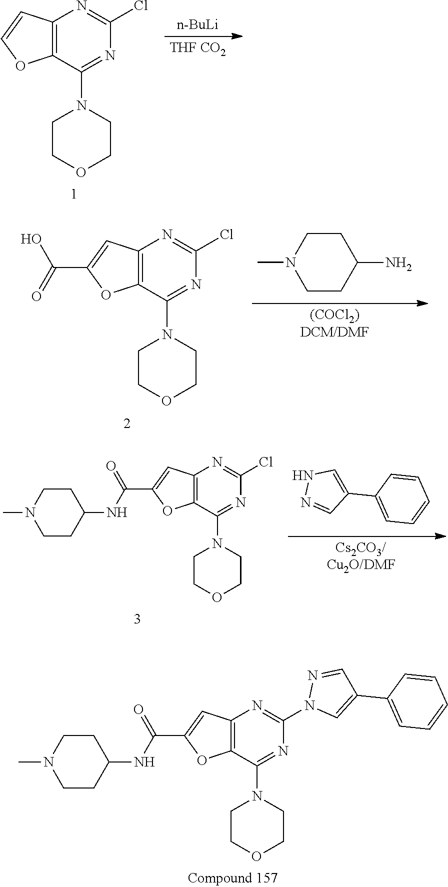

### 1) Synthesis of 2-chloro-4-morpholinofuro[3,2-d]pyrimidine-6-carboxylic acid

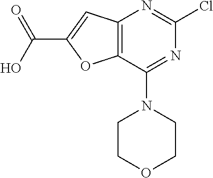 [SMILES: O=C(O)c1cc2nc(Cl)nc(N3CCOCC3)c2o1]

To a solution of 2-chloro-4-morpholinofuro[3,2-d]pyrimidine (2.4 g, 10 mmol) in anhydrous THE (40 mL) at −78° C. under N₂ was added n-BuLi (5.2 mL, 2.5 M, 13 mmol) dropwise. The reaction mixture was stirred at that temperature for 1 h. To the above solution was added dry ice (4.4 g, 100 mmol) in one portion. The resulting reaction mixture was stirred at that temperature for 3 h. The completion of the reaction was monitored by TLC. The reaction was quenched with water and the pH was adjusted to 5 using 1 N HCl solution. The aqueous solution was extracted with DCM (3×80 mL). The combined organic phase was dried over anhydrous Na₂SO₄, filtrated and concentrated under reduce pressure. The resulting residue was slurry in Et₂O to provide 2-chloro-4-morpholinofuro[3,2-d]pyrimidine-6-carboxylic acid (2.92 g, 10.3 mmol) as a yellow solid. LC-MS (ESI+): m/z 284/286 (MW). ¹HNMR (300 MHz, CDCl₃) δ 7.58 (s, 1H), 4.04-3.95 (m, 4H), 3.78-3.76 (m, 4H).

### 2) Synthesis of 2-chloro-N-(1-methylpiperidin-4-yl)-4-morpholinofuro[3,2-d]pyrimidine-6-carboxamide

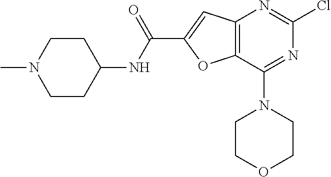 [SMILES: CN1CCC(NC(=O)c2cc3nc(Cl)nc(N4CCOCC4)c3o2)CC1]

To a solution of 2-chloro-4-morpholinofuro[3,2-d]pyrimidine-6-carboxylic acid (400 mg, 1.42 mmol) in DCM was added oxalyl dichloride (360 mg, 2.84 mmol) and one drop of DMF. The mixture was stirred at rt for 2 h. The solution was concentrated and the resulting residue was dissolved in DCM (15 mL). To the solution was added 1-methylpiperidin-4-amine (178 mg, 1.56 mmol) and followed by DIEA (107 mg, 2.84 mmol). The completion of the reaction was monitored by TLC. The reaction mixture was quenched with water and extracted with DCM (3×20 mL). The combined organic phase was dried over anhydrous Na₂SO₄, filtrated and concentrated under reduce pressure. The residue was purified by silica gel column chromatography with a gradient elution of 5% MeOH/DCM to 10% MeOH/DCM to provide 2-chloro-N-(1-methylpiperidin-4-yl)-4-morpholinofuro[3,2-d]pyrimidine-6-carboxamide (320 mg, 0.84 mmol) as a yellow solid. LC-MS (ESI+): m/z 380/382 (MH⁺).

### 3) Synthesis of N-(1-methylpiperidin-4-yl)-4-morpholino-2-(4-phenyl-1H-pyrazol-1-yl)furo[3,2-d]pyrimidine-6-carboxamide

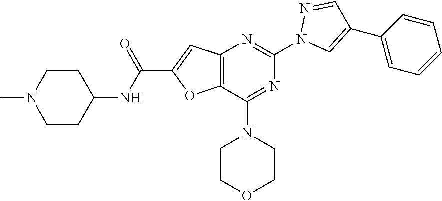 [SMILES: CN1CCC(NC(=O)c2cc3nc(-n4cc(-c5ccccc5)cn4)nc(N4CCOCC4)c3o2)CC1]

To a solution of 2-chloro-N-(1-methylpiperidin-4-yl)-4-morpholinofuro[3,2-d]pyrimidine-6-carboxamide (100 mg, 0.26 mmol) in DMF (4 mL) was added 4-phenyl-1H-pyrazole (42 mg, 0.29 mmol), Cs₂CO₃ (172 mg, 0.53 mmol) and Cu₂O (4 mg, 0.026 mmol). The reaction was stirred at 110° C. overnight. The reaction mixture was quenched with water (10 mL). The aqueous solution was extracted with DCM (3×10 mL). The combined organic phase was dried over anhydrous Na₂SO₄, filtrated and concentrated. The resulting residue was purified by preparative HPLC to provide N-(1-methylpiperidin-4-yl)-4-morpholino-2-(4-phenyl-1H-pyrazol-1-yl)furo[3,2-d]pyrimidine-6-carboxamide (32 mg, 0.065 mmol) as a white solid. LC-MS (ESI+): m/z 488 (MH⁺). ¹HNMR (300 MHz, CDCl₃) δ 8.71 (s, 1H), 8.08 (s, 1H), 7.61 (d, J=7.5 Hz, 2H), 7.44 (s, 1H), 7.38-7.26 (m, 3H), 6.33 (d, J=7.8 Hz, 1H), 4.21-4.03 (m, 5H), 3.94-3.88 (m, 4H), 2.99-2.88 (m, 2H), 2.40 (s, 3H), 2.33-2.22 (m, 2H), 2.19-2.05 (m, 2H), 1.87-1.75 (m, 2H).

### Example 39: Compound 179 Using General Synthetic Route 39

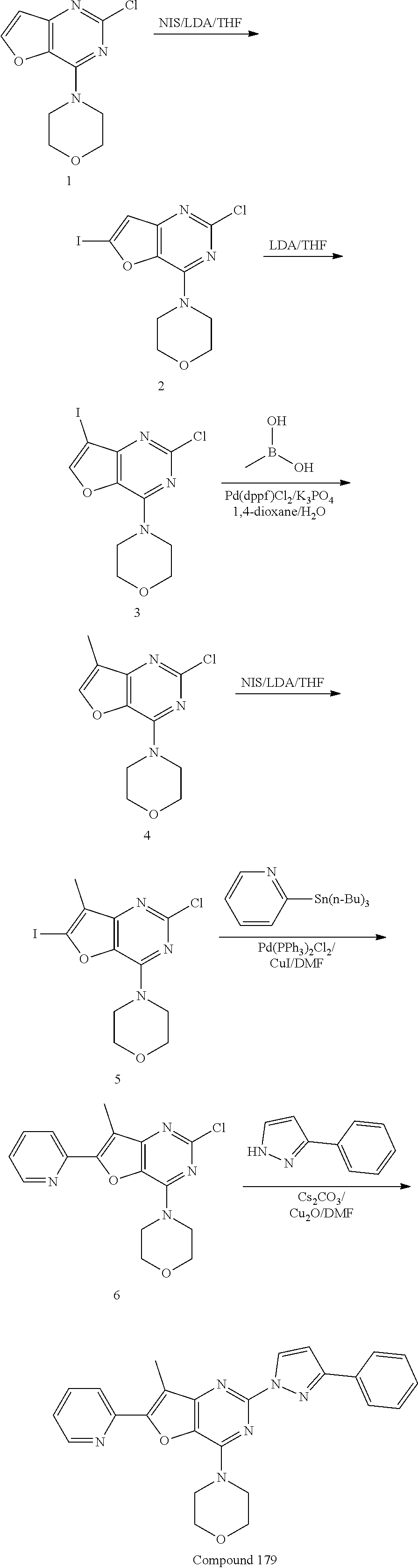

### 1) Synthesis of 2-chloro-6-iodo-4-morpholinofuro[3, 2-d]pyrimidine

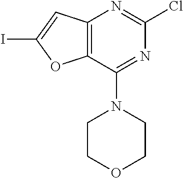 [SMILES: Clc1nc(N2CCOCC2)c2oc(I)cc2n1]

To a solution of 2-chloro-4-morpholinofuro[3,2-d]pyrimidine (2.0 g, 0.83 mmol) in THE (30 mL) at −78° C. under N₂ was added LDA (1.33 mL, 2M, 2.66 mmol). After stirred at −78° C. for 1 h, to the solution was added a solution of NIS (2.25 g, 1.0 mmol) in THF (10 mL). The completion of the reaction was monitored by TLC. The reaction mixture was quenched with water (50 mL). The aqueous solution was extracted with DCM (3×50 mL). The combined organic phase was dried over anhydrous Na₂SO₄, filtrated and concentrated under reduce pressure. The resulting residue was purified by silica gel column chromatography with a gradient elution of 5% EtOAc/PE to 10% EtOAc/PE to provide 2-chloro-6-iodo-4-morpholinofuro[3,2-d]pyrimidine (1.6 g, 4.4 mmol) as a yellow solid. LC-MS (ESI+): m/z 366/368 (MH⁺). ¹HNMR (300 MHz, CDCl₃) δ 6.97 (s, 1H), 4.01-3.98 (m, 4H), 3.85-3.82 (m, 4H).

### 2) Synthesis of 2-chloro-7-iodo-4-morpholinofuro[3,2-d]pyrimidine

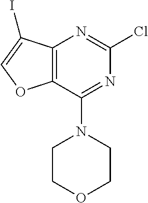 [SMILES: Clc1nc(N2CCOCC2)c2occ(I)c2n1]

To a solution of 2-chloro-6-iodo-4-morpholinofuro[3,2-d]pyrimidine (5.0 g, 13.7 mmol) in THF (30 mL) at −78° C. under N₂ was added LDA (14 mL, 2M, 27.4 mmol). After addition, the reaction mixture was stirred at −78° C. for 1 h. The completion of the reaction was monitored by TLC. The reaction mixture was quenched with H₂O (100 mL). The aqueous solution was extracted with DCM (3×200 mL). The combined organic phase was dried over anhydrous Na₂SO₄, filtrated and concentrated under reduce pressure. The resulting residue was purified by silica gel column chromatography with a gradient elution of 10% EtOAc/PE to 30% EtOAc/PE to provide 2-chloro-7-iodo-4-morpholinofuro[3,2-d]pyrimidine (2.5 g, 6.85 mmol) as a white solid. LC-MS (ESI+): m/z 366/368 (MH⁺). ¹HNMR (300 MHz, CDCl₃) δ 7.77 (s, 1H), 4.04-3.94 (m, 4H), 3.85-3.81 (m, 4H).

### 3) Synthesis of 2-chloro-7-methyl-4-morpholinofuro[3,2-d]pyrimidine

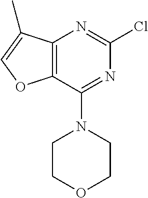 [SMILES: Cc1coc2c(N3CCOCC3)nc(Cl)nc12]

To a solution of 2-chloro-7-iodo-4-morpholinofuro[3,2-d]pyrimidine (500 mg, 0.14 mmol) in DME/H₂O (2/1, 30 mL) was added methylboronic acid (250 mg, 4.2 mmol), K₃PO₄ (440 mg, 2.1 mmol) and Pd(PPh₃)₄ (150 mg, 0.14 mmol). The reaction was stirred at 120° C. for overnight. The completion of the reaction was monitored by TLC. The reaction mixture was quenched with water (50 mL). The aqueous solution was extracted with EtOAc (2×50 mL). The combined organic phase was dried over anhydrous Na₂SO₄, filtrated and concentrated. The resulting residue was purified by silica gel column chromatography with a gradient elution of 10% EtOAc/PE to 30% EtOAc/PE to provide 2-chloro-7-methyl-4-morpholinofuro[3,2-d]pyrimidine (350 mg, 1.4 mmol) as a white solid. LC-MS (ESI+): m/z 254/256 (MH⁺).

### 4) Synthesis of 2-chloro-6-iodo-7-methyl-4-morpholinofuro[3,2-d]pyrimidine

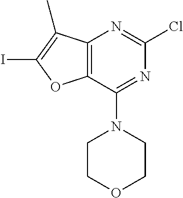 [SMILES: Cc1c(I)oc2c(N3CCOCC3)nc(Cl)nc12]

To a solution of 2-chloro-7-methyl-4-morpholinofuro[3,2-d]pyrimidine (350 mg, 1.38 mmol) in THF (30 mL) at −78° C. under N₂ was added LDA (1.4 mL, 2 M, 2.76 mmol). After stirred at −78° C. for 1 h, to the solution was added a solution of NIS (374 mg, 1.66 mmol) in THF (5 mL). The completion of the reaction was monitored by TLC. The reaction mixture was quenched with water (50 mL). The aqueous solution was extracted with EtOAc (3×20 mL). The combined organic phase was dried over anhydrous Na₂SO₄, filtrated and concentrated under reduce pressure. The resulting residue was purified by silica gel column chromatography with a gradient elution of 10% EtOAc/PE to 30% EtOAc/PE to provide 2-chloro-6-iodo-7-methyl-4-morpholinofuro[3,2-d]pyrimidine (280 mg, 2.55 mmol) as yellow solid. LC-MS (ESI+): m/z 380/382 (MHI).

### 5) Synthesis of 2-chloro-7-methyl-4-morpholino-6-(pyridin-2-yl)furo[3, 2-d]pyrimidine

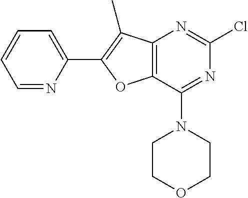 [SMILES: Cc1c(-c2ccccn2)oc2c(N3CCOCC3)nc(Cl)nc12]

A solution of 2-chloro-6-iodo-7-methyl-4-morpholinofuro[3,2-d]pyrimidine (200 mg, 0.53 mmol), 2-(tributylstannyl)pyridine (389 mg, 1.06 mmol) and Pd(PPh₃)₄ (61 mg, 0.053 mmol) in toluene (5 mL) was heated to 90° C. overnight. The completion of the reaction was monitored by TLC. The reaction mixture was diluted with water and extracted with DCM/MeOH (15/1, 3×50 mL). The combined organic phase was dried over anhydrous Na₂SO₄, filtrated and concentrated under reduce pressure. The resulting residue was purified by silica gel column chromatography with a gradient elution of 30% EtOAc/PE to 50% EtOAc/PE to provide 2-chloro-7-methyl-4-morpholino-6-(pyridin-2-yl)furo[3,2-d]pyrimidine (70 mg, 0.21 mmol) as a white solid. LC-MS (ESI+): m/z 331/333 (MH⁺). ¹HNMR (300 MHz, CDCl₃) β 8.67 (d, J=4.8 Hz, 1H), 7.78-7.67 (m, 2H), 7.31-7.25 (m, 1H), 4.06-4.01 (m, 4H), 3.82-3.79 (m, 4H), 2.59 (s, 3H).

### 6) Synthesis of 7-methyl-4-morpholino-2-(3-phenyl-1H-pyrazol-1-yl)-6-(pyridin-2-yl)furo[3,2-d]pyrimidine

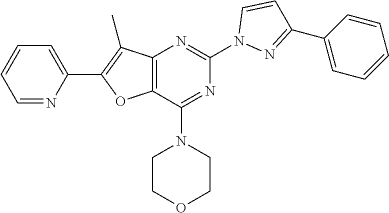 [SMILES: Cc1c(-c2ccccn2)oc2c(N3CCOCC3)nc(-n3ccc(-c4ccccc4)n3)nc12]

To a solution of 2-chloro-7-methyl-4-morpholino-6-(pyridin-2-yl)furo[3,2-d]pyrimidine (80 mg, 0.24 mmol) in DMF (10 mL) was added 3-phenyl-1H-pyrazole (42 mg, 0.29 mmol) and Cs₂CO₃ (160 mg, 0.49 mmol). The reaction was stirred at 100° C. overnight. The completion of the reaction was monitored by TLC. The reaction mixture was quenched with water (10 mL). The aqueous solution was extracted with EtOAc (3×20 mL). The combined organic phase was dried over anhydrous Na₂SO₄, filtrated and concentrated. The resulting residue was purified by silica gel column chromatography with a gradient elution of 2% MeOH/DCM to 5% MeOH/DCM to provide 7-methyl-4-morpholino-2-(3-phenyl-1H-pyrazol-1-yl)-6-(pyridin-2-yl) furo[3,2-d]pyrimidine (28.6 mg, 0.065 mmol) as white solid. LC-MS (ESI+): m/z 439 (MH⁺). ¹HNMR (300 MHz, CDCl₃) δ 8.76 (d, J=1.5 Hz, 1H), 8.62 (d, J=2.7 Hz, 1H), 8.02 (d, J=7.2 Hz, 2H), 7.83-7.81 (m, 2H), 7.46-7.41 (m, 2H), 7.37-7.27 (m, 2H), 6.79 (d, J=2.7 Hz, 1H), 4.21-4.15 (m, 4H), 3.96-3.90 (m, 4H), 2.77 (s, 3H).

### Example 40: Compound 147 Using General Synthetic Route 40

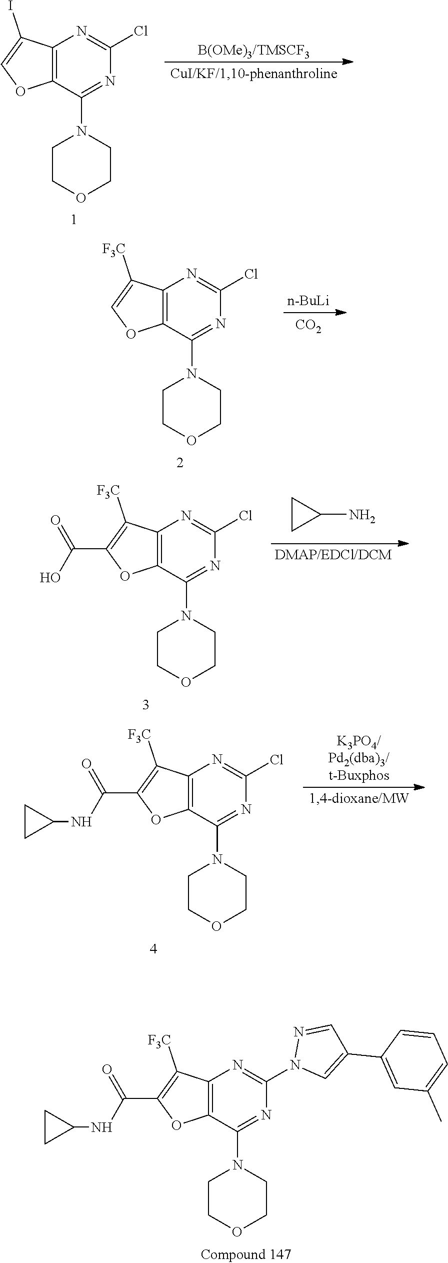

### 1) Synthesis of 2-chloro-4-morpholino-7-(trifluoromethyl)furo[3,2-d]pyrimidine

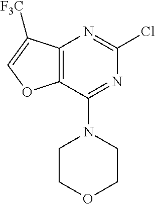 [SMILES: FC(F)(F)c1coc2c(N3CCOCC3)nc(Cl)nc12]

To a solution of 2-chloro-7-iodo-4-morpholinofuro[3,2-d]pyrimidine (300 mg, 0.82 mmol) in anhydrous DMSO (6 mL) was added KF (144 mg, 2.47 mmol), CuI (30 mg, 0.16 mmol), 1,10-phenanthroline (30 mg, 0.16 mmol), B(OMe)₃ (252 mg, 2.47 mmol) and TMSCF₃ (348 mg, 2.47 mmol). The reaction mixture was stirred at 50° C. for 2 h. The reaction mixture was quenched with water (10 mL). The aqueous solution was extracted with DCM (3×20 mL). The combined organic phase was dried over anhydrous Na₂SO₄, filtrated and concentrated under reduce pressure. The resulting residue was purified by silica gel column chromatography with a gradient elution of 10% EtOAc/PE to 30% EtOAc/PE to provide 2-chloro-4-morpholino-7-(trifluoromethyl)furo[3,2-d]pyrimidine (98 mg, 0.32 mmol) as a yellow solid. LC-MS (ESI+): m/z 308/310 (MH⁺).

### 2) Synthesis of 2-chloro-4-morpholino-7-(trifluoromethyl)furo[3,2-d]pyrimidine-6-carboxylic acid

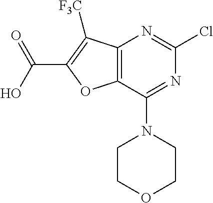 [SMILES: O=C(O)c1oc2c(N3CCOCC3)nc(Cl)nc2c1C(F)(F)F]

To a solution of 2-chloro-4-morpholino-7-(trifluoromethyl)furo[3,2-d]pyrimidine (490 mg, 1.60 mmol) in anhydrous THE (100 mL) at −78° C. under N₂ was added n-BuLi (0.96 mL, 2.5 M, 2.4 mmol) dropwise. The reaction mixture was stirred at that temperature for 1 h. To the above solution was added dry ice (705 mg, 16 mmol) in one portion. The resulting reaction mixture was stirred at that temperature for 1 h. The completion of the reaction was monitored by TLC. The reaction was quenched with water and the pH was adjusted to 5 using 1 N HCl solution. The aqueous solution was extracted with DCM (2×20 mL). The combined organic phase was dried over anhydrous Na₂SO₄, filtrated and concentrated under reduce pressure. The resulting residue was slurry in Et₂O to provide 2-chloro-4-morpholino-7-(trifluoromethyl)furo[3,2-d]pyrimidine-6-carboxylic acid (270 mg, 0.77 mmol) as a brown solid. LC-MS (ESI+): m/z 352/354 (MH⁺).

### 3) Synthesis of 2-chloro-N-cyclopropyl-4-morpholino-7-(trifluoromethyl)furo[3,2-d]pyrimidine-6-carboxamide

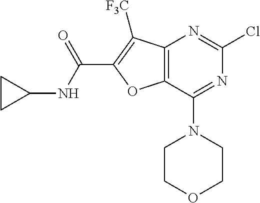 [SMILES: O=C(NC1CC1)c1oc2c(N3CCOCC3)nc(Cl)nc2c1C(F)(F)F]

To a solution of 2-chloro-4-morpholino-7-(trifluoromethyl)furo[3,2-d]pyrimidine-6-carboxylic acid (270 mg, 0.77 mmol) in DCM (10 mL) was added cyclopropanamine (43 mg, 0.77 mmol), EDCl (175 mg, 0.92 mmol) and DMAP (112 mg, 0.92 mmol). The reaction was stirred at rt overnight. The completion of the reaction was monitored by TLC. The reaction was quenched with water (10 mL) and extracted with DCM (3×10 mL). The combined organic phase was dried over anhydrous Na₂SO₄, filtrated and concentrated under reduced pressure. The resulting residue was purified by silica gel column chromatography with a gradient elution of 10% EtOAc/PE to 30% EtOAc/PE to provide 2-chloro-N-cyclopropyl-4-morpholino-7-(trifluoromethyl)furo[3,2-d]pyrimidine-6-carboxamide (60 mg, 0.15 mmol) as white solid. LC-MS (ESI+): m/z 391/393 (MH⁺).

### 4) Synthesis of N-cyclopropyl-4-morpholino-2-(4-(m-tolyl)-1H-pyrazol-1-yl)-7-(trifluoromethyl)furo[3,2-d]pyrimidine-6-carboxamide

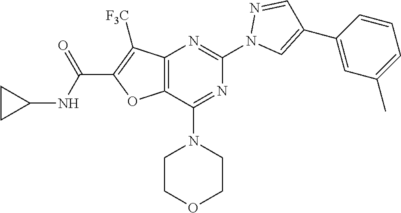 [SMILES: Cc1cccc(-c2cnn(-c3nc(N4CCOCC4)c4oc(C(=O)NC5CC5)c(C(F)(F)F)c4n3)c2)c1]

To a solution of 2-chloro-N-cyclopropyl-4-morpholino-7-(trifluoromethyl)furo[3,2-d]pyrimidine-6-carboxamide (60 mg, 0.15 mmol) in 1,4-dioxane (2 mL) was added 4-(m-tolyl)-1H-pyrazole (30 mg, 0.18 mmol), Pd₂(dba)₃ (15 mg, 0.015 mmol), t-Buxphos (15 mg, 0.015 mmol) and K₃PO₄ (50 mg, 0.63 mmol). The reaction was stirred at 80° C. under microwave for 30 min. The reaction mixture was diluted with water and extracted with DCM (3×20 mL). The combined organic phase was dried over anhydrous Na₂SO₄, filtrated and concentrated under reduce pressure. The resulting residue was purified by silica gel column chromatography with a gradient elution of 1% MeOH/DCM to 2% MeOH/DCM to provide N-cyclopropyl-4-morpholino-2-(4-(m-tolyl)-1H-pyrazol-1-yl)-7-(trifluoromethyl)furo[3,2-d]pyrimidine-6-carboxamide (13 mg, 0.025 mmol) as a yellow solid. LC-MS (ESI+): m/z 513 (MH⁺). ¹HNMR (300 MHz, CDCl₃) δ 8.77 (s, 1H), 8.08 (s, 1H), 7.61-7.42 (m, 2H), 7.35-7.27 (m, 1H), 7.11-7.09 (m, 1H), 6.48 (s, 1H), 4.17-4.12 (m, 4H), 3.92-3.88 (m, 4H), 2.95-2.90 (m, 1H), 2.41 (s, 3H), 0.98-0.93 (m, 2H), 0.75-0.69 (m, 2H).

### Example 41: Compound 204 Using General Synthetic Route 41

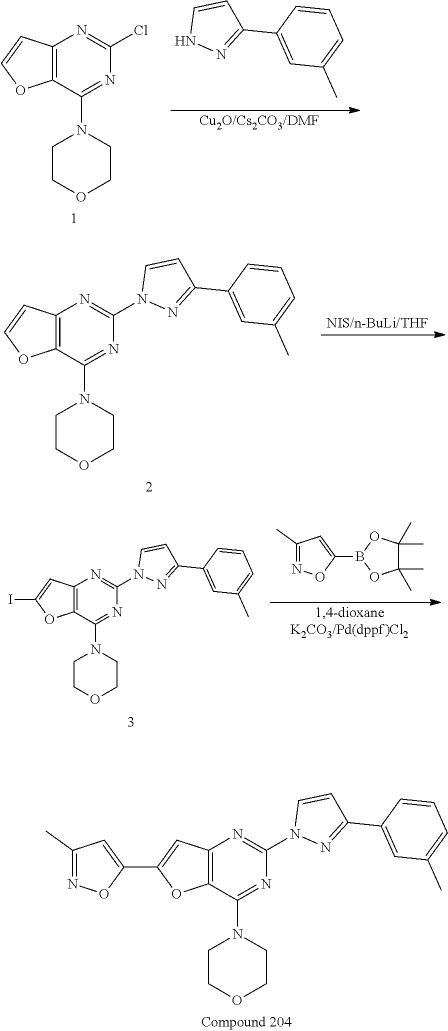

### 1) Synthesis of 4-morpholino-2-(3-(m-tolyl)-1H-pyrazol-1-yl)furo[3,2-d]pyrimidine

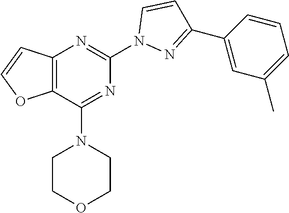 [SMILES: Cc1cccc(-c2ccn(-c3nc(N4CCOCC4)c4occc4n3)n2)c1]

To a solution of 2-chloro-4-morpholinofuro[3,2-d]pyrimidine (300 mg, 1.25 mmol) in DMF (4 mL) was added 3-(m-tolyl)-1H-pyrazole (237 mg, 0.35 mmol), Cs₂CO₃ (815 mg, 2.5 mmol) and CuI (24 mg, 0.125 mmol). The reaction mixture was stirred at 110° C. overnight. The reaction mixture was quenched with water (10 mL). The aqueous solution was extracted with EtOAc (3×10 mL). The combined organic phase was dried over anhydrous Na₂SO₄, filtrated and concentrated under reduce pressure. The resulting residue was purified by silica gel column chromatography with a gradient elution of 10% EtOAc/PE to 30% EtOAc/PE to provide 4-morpholino-2-(3-(m-tolyl)-1H-pyrazol-1-yl)furo[3,2-d]pyrimidine (269 mg, 0.75 mmol) as a white solid. LC-MS (ESI+): m/z 362 (MH⁺). ¹HNMR (300 MHz, DMSO-d₆) δ 8.75 (d, J=2.7 Hz, 1H), 8.38 (d, J=1.8 Hz, 1H), 7.82 (s, 1H), 7.77 (d, J=7.8 Hz, 1H), 7.39-7.34 (m, 1H), 7.21 (d, J=7.8 Hz, 1H), 7.13 (d, J=1.8 Hz, 1H), 7.07 (d, J=2.7 Hz, 1H), 4.08-4.07 (m, 4H), 3.82-3.81 (m, 4H), 2.35 (s, 3H).

### 2) Synthesis of 6-iodo-4-morpholino-2-(3-(m-tolyl)-1H-pyrazol-1-yl)furo[3, 2-d]pyrimidine

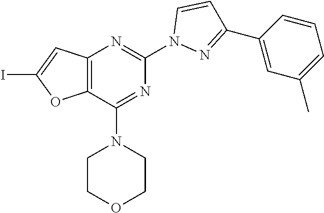 [SMILES: Cc1cccc(-c2ccn(-c3nc(N4CCOCC4)c4oc(I)cc4n3)n2)c1]

To a solution of 4-morpholino-2-(3-(m-tolyl)-1H-pyrazol-1-yl)furo[3,2-d]pyrimidine (180 mg, 0.5 mmol) in anhydrous THE (40 mL) at −78° C. under N₂ was added n-BuLi (0.24 mL, 2.5 M, 0.6 mmol) dropwise. The reaction mixture was stirred at that temperature for 1 h. To the solution was added a solution of NIS (135 mg, 0.6 mmol) in THE (3 mL) dropwise. The resulting reaction mixture was stirred at that temperature for 2 h. The completion of the reaction was monitored by TLC. The reaction was quenched with water. The aqueous solution was extracted with DCM (3×20 mL). The combined organic phase was dried over anhydrous Na₂SO₄, filtrated and concentrated under reduce pressure. The resulting residue was purified by silica gel column chromatography with a gradient elution of 20% EtOAc/PE to 50% EtOAc/PE to provide 6-iodo-4-morpholino-2-(3-(m-tolyl)-1H-pyrazol-1-yl)furo[3,2-d]pyrimidine (70 mg, 0.14 mmol) as a white solid. LC-MS (ESI+): m/z 488 (MH⁺).

### 3) Synthesis of 6-(3-methylisoxazol-5-yl)-4-morpholino-2-(3-(m-tolyl)-1H-pyrazol-1-yl)furo[3,2-d]pyrimidine

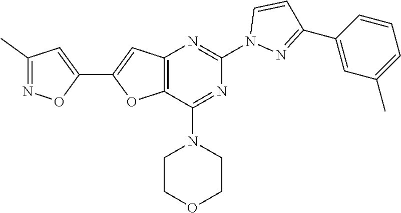 [SMILES: Cc1cccc(-c2ccn(-c3nc(N4CCOCC4)c4oc(-c5cc(C)no5)cc4n3)n2)c1]

A suspension of 6-iodo-4-morpholino-2-(3-(m-tolyl)-1H-pyrazol-1-yl)furo[3,2-d]pyrimidine (70 mg, 0.14 mmol), 3-methyl-5-(4,4,5,5-tetramethyl-1,3,2-dioxaborolan-2-yl)isoxazole (36 mg, 0.172 mmol), K₂CO₃ (60 mg, 0.43 mmol) and Pd(dppf)Cl₂ (11 mg, 0.014 mmol) in 1,4-dioxane/H₂O (8/1, 10 mL) was heated to 90° C. for 2 h under N₂. The completion of the reaction was monitored by TLC. The reaction was concentrated directly. The resulting residue was purified by silica gel column chromatography with a gradient elution of 2% MeOH/DCM to 6% MeOH/DCM to provide 6-(3-methylisoxazol-5-yl)-4-morpholino-2-(3-(m-tolyl)-1H-pyrazol-1-yl)furo[3,2-d]pyrimidine (22 mg, 0.05 mmol) as a light yellow solid. LC-MS (ESI+): m/z 443 (MH⁺). ¹HNMR (300 MHz, CDCl₃) δ 8.54 (d, J=2.7 Hz, 1H), 7.90 (s, 1H), 7.76 (d, J=7.5 Hz, 1H), 7.39-7.26 (m, 2H), 7.17 (d, J=7.8 Hz, 1H), 6.78 (d, J=2.7 Hz, 1H), 6.58 (s, 1H), 4.17-4.13 (m, 4H), 3.95-3.91 (m, 4H), 2.42 (s, 6H).

### Example 42: Compound 212 Using General Synthetic Route 42

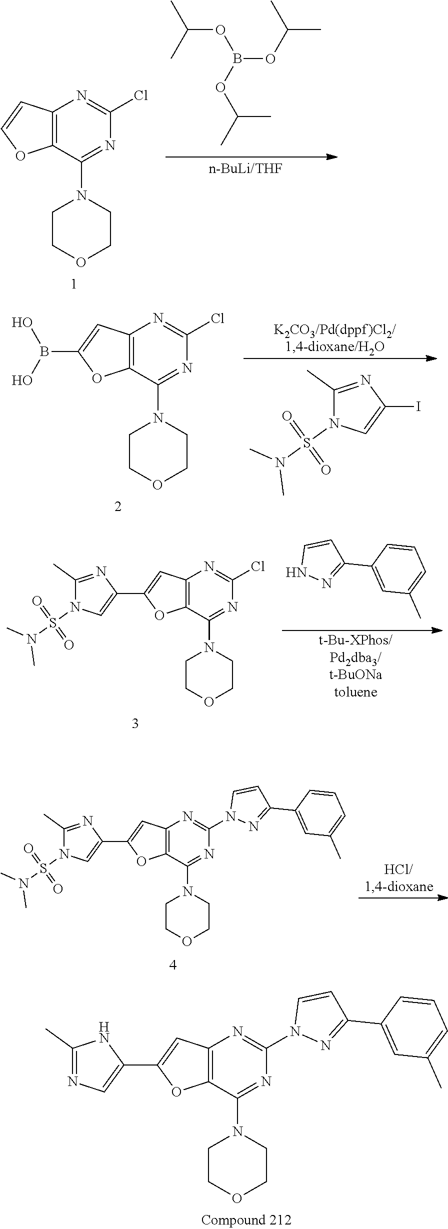

### 1) Synthesis of (2-chloro-4-morpholinofuro[3,2-d]pyrimidin-6-yl)boronic acid

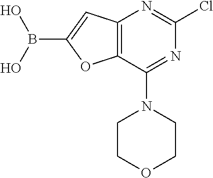 [SMILES: OB(O)c1cc2nc(Cl)nc(N3CCOCC3)c2o1]

To a solution of 2-chloro-4-morpholinofuro[3,2-d]pyrimidine (200 mg, 0.84 mmol) in THF (30 mL) at −78° C. under N₂ was added n-BuLi (0.4 mL, 2.5 M, 1.00 mmol). After stirred at −78° C. for 1 h, to the solution was added a solution of triisopropyl borate (190 mg, 1.00 mmol) in THF (2 mL). The reaction mixture was stirred at rt overnight. The reaction mixture was quenched with water (0.1 mL) and a large amount of white solid was precipitated. After filtration, the filter cake was washed with 4 mL THF and dried in vacuum to provide (2-chloro-4-morpholinofuro[3,2-d]pyrimidin-6-yl)boronic acid (150 mg, 0.53 mmol) as white solid. LC-MS (ESI+): m/z 284/286 (MH⁺). ¹HNMR (300 MHz, CD₃OD) δ 6.58 (s, 1H), 4.12-4.05 (m, 4H), 3.84-3.78 (m, 4H).

### 2) Synthesis of 4-(2-chloro-4-morpholinofuro[3,2-d]pyrimidin-6-yl)-N,N,2-trimethyl-1H-imidazole-1-sulfonamide

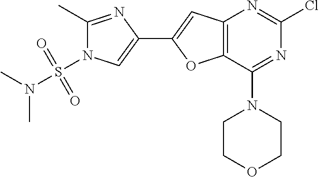 [SMILES: Cc1nc(-c2cc3nc(Cl)nc(N4CCOCC4)c3o2)cn1S(=O)(=O)N(C)C]

A suspension of 4-iodo-N,N,2-trimethyl-1H-imidazole-1-sulfonamide (367 mg, 1.17 mmol), (2-chloro-4-morpholinofuro[3,2-d]pyrimidin-6-yl)boronic acid (300 mg, 1.06 mmol), K₂CO₃ (439 mg, 3.18 mmol) and Pd(dppf)Cl₂ (78 mg, 0.106 mmol) in 1,4-dioxane/H₂O (8/1, 20 mL) was heated to 90° C. for 1 h under N₂. The completion of the reaction was monitored by TLC. The reaction was concentrated directly under reduce pressure. The resulting residue was purified by silica gel column chromatography with a gradient elution of 1% MeOH/DCM to 2% MeOH/DCM to provide 4-(2-chloro-4-morpholinofuro[3,2-d]pyrimidin-6-yl)-N,N,2-trimethyl-1H-imidazole-1-sulfonamide (340 mg, 0.80 mmol) as a yellow solid. LC-MS (ESI+): m/z 427/429 (MH⁺). ¹HNMR (300 MHz, CDCl₃) δ 7.62 (s, 1H), 7.02 (s, 1H), 4.09-4.03 (m, 4H), 3.91-3.84 (m, 4H), 3.04 (s, 6H), 2.69 (s, 3H).

### 3) Synthesis of N,N,2-trimethyl-4-(4-morpholino-2-(3-(m-tolyl)-1H-pyrazol-1-yl)furo[3,2-d]pyrimidin-6-yl)-1H-imidazole-1-sulfonamide

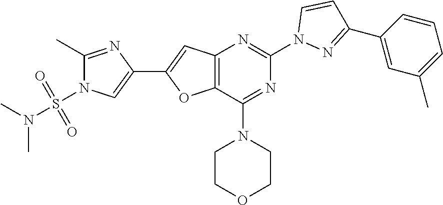 [SMILES: Cc1cccc(-c2ccn(-c3nc(N4CCOCC4)c4oc(-c5cn(S(=O)(=O)N(C)C)c(C)n5)cc4n3)n2)c1]

A suspension of 4-(2-chloro-4-morpholinofuro[3,2-d]pyrimidin-6-yl)-N,N,2-trimethyl-1H-imidazole-1-sulfonamide (110 mg, 0.26 mmol), 3-(m-tolyl)-1H-pyrazole (49 mg, 0.31 mmol), t-BuONa (0.52 mL, 1M, 0.52 mmol), Pd₂(dba)₃ (14.8 mg, 0.066 mmol) and t-BuXphos (80 mg, 0.18 mmol) in toluene (10 mL) under N₂ was heated to 100° C. for 1 h. The completion of the reaction was monitored by TLC. The reaction was concentrated directly. The residue was purified by silica gel column chromatography with a gradient elution of 1% MeOH/DCM to 2% MeOH/DCM to provide to provide 50 mg of impure product. After further slurry in MeOH to provide N,N,2-trimethyl-4-(4-morpholino-2-(3-(m-tolyl)-1H-pyrazol-1-yl)furo[3,2-d] pyrimidin-6-yl)-1H-imidazole-1-sulfonamide (35 mg, 0.064 mmol) as a white solid. LC-MS (EMS+): m/z 549 (MH⁺).

### 4) Synthesis of 6-(2-methyl-1H-imidazol-5-yl)-4-morpholino-2-(3-(m-tolyl)-1H-pyrazol-1-yl)furo[3,2-d]pyrimidine

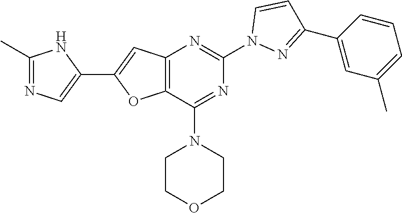 [SMILES: Cc1cccc(-c2ccn(-c3nc(N4CCOCC4)c4oc(-c5cnc(C)[nH]5)cc4n3)n2)c1]

To a solution of N,N,2-trimethyl-4-(4-morpholino-2-(3-(m-tolyl)-1H-pyrazol-1-yl) furo[3,2-d]pyrimidin-6-yl)-1H-imidazole-1-sulfonamide (35 mg, 0.064 mmol) in 1,4-dioxane (4 mL) was added conc. HCl (0.3 mL). The reaction mixture was stirred at 80° C. for 2 h. The completion of the reaction was monitored by TLC. The reaction mixture was quenched with saturated NaHCO₃ solution to pH=8. A large amount of solid was precipitated. After filtration, the filter cake was slurry in Et₂O to provide N,N,2-trimethyl-4-(4-morpholino-2-(3-(m-tolyl)-1H-pyrazol-1-yl)furo[3,2-d]pyrimidin-6-yl)-1H-imidazole-1-sulfonamide (20 mg, 0.045 mmol) as a light yellow solid. LC-MS (ESI+): m/z 442 (MH⁺). ¹HNMR (300 MHz, CDCl₃) δ 8.53 (d, J=2.7 Hz, 1H), 7.82 (s, 1H), 7.70 (d, J=7.5 Hz, 1H), 7.43 (s, 1H), 7.31-7.26 (m, 1H), 7.14 (d, J=7.8 Hz, 1H), 7.04 (s, 1H), 6.76 (d, J=2.7 Hz, 1H), 4.15-4.11 (m, 4H), 3.95-3.91 (m, 4H), 2.51 (s, 3H), 2.41 (s, 3H).

### Example 43: Compound 144 Using General Synthetic Route 43

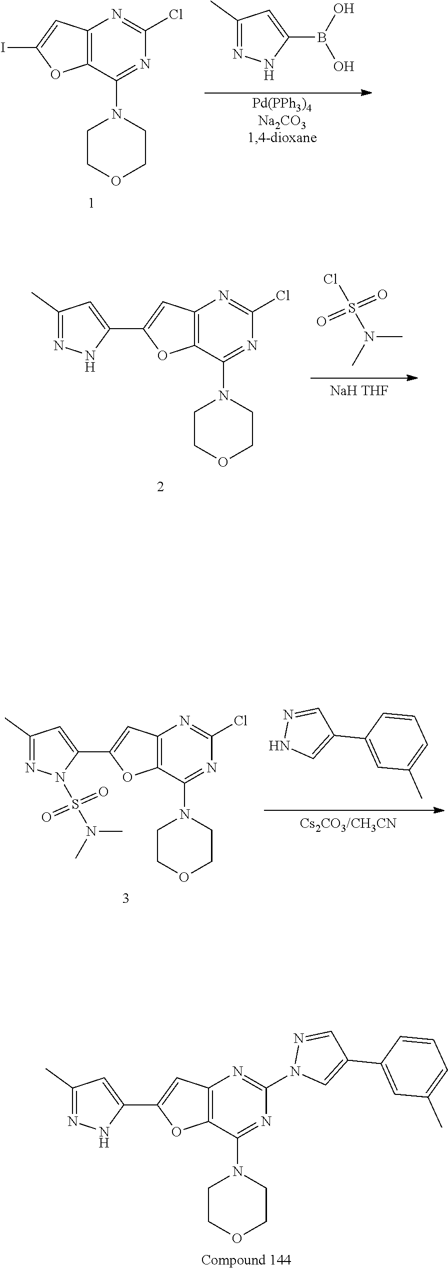

### 1) Synthesis of 2-chloro-6-(3-methyl-1H-pyrazol-5-yl)-4-morpholinofuro[3,2-d]pyrimidine

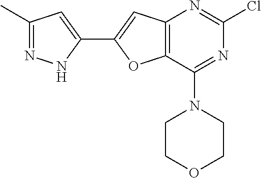 [SMILES: Cc1cc(-c2cc3nc(Cl)nc(N4CCOCC4)c3o2)[nH]n1]

A suspension of 2-chloro-6-iodo-4-morpholinofuro[3,2-d]pyrimidine (400 mg, 1.08 mmol), (3-methyl-1H-pyrazol-5-yl)boronic acid (154 mg, 1.1 mmol), Na₂CO₃ (232 mg, 2.16 mmol) and Pd(PPh₃)₄ (12 mg, 0.01 mmol) in 1,4-dioxane/H₂O (8 mL, 4:1) under N₂ was heated to 50° C. for 2 h. The completion of the reaction was monitored by TLC. The reaction mixture was concentrated directly. The resulting residue was purified by silica gel column chromatography with a gradient elution of 33% EtOAc/PE to 50% EtOAc/PE to provide 2-chloro-6-(3-methyl-1H-pyrazol-5-yl)-4-morpholinofuro[3,2-d]pyrimidine (180 mg, 0.57 mmol) as a yellow solid. LC-MS (ESI+): m/z 320/322 (MH⁺).

### 2) Synthesis of 5-(2-chloro-4-morpholinofuro[3,2-d]pyrimidin-6-yl)-N,N,3-trimethyl-1H-pyrazole-1-sulfonamide

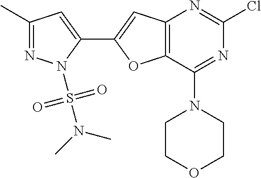 [SMILES: Cc1cc(-c2cc3nc(Cl)nc(N4CCOCC4)c3o2)n(S(=O)(=O)N(C)C)n1]

To a solution of 2-chloro-6-(3-methyl-1H-pyrazol-5-yl)-4-morpholinofuro[3,2-d]pyrimidine (180 mg, 0.57 mmol) in THE at 0° C. was added NaH (27 mg, 0.67 mmol). The mixture was stirred at 0° C. for 0.5 h. To the above mixture at 0° C. was added Dimethylsulfamoyl chloride (104 mg, 0.73 mmol) dropwise. After addition, the reaction mixture was stirred at rt for 3.5 h. The completion of the reaction was monitored by TLC. The reaction mixture was quenched with water (20 mL). The aqueous solution was extracted with EtOAc (3×10 mL). The combined organic phase was dried over anhydrous Na₂SO₄, filtrated and concentrated under reduce pressure. The resulting residual was slurry in Et₂O to provide crude 5-(2-chloro-4-morpholinofuro[3,2-d]pyrimidin-6-yl)-N,N,3-trimethyl-1H-pyrazole-1-sulfonamide (270 mg, 0.63 mmol) as a yellow solid. LC-MS (ESI+): m/z 427/429 (MH⁺). ¹HNMR (300 MHz, CDCl₃) δ 7.06 (s, 1H), 6.49 (s, 1H), 4.13-4.04 (m, 4H), 3.87-3.81 (m, 4H), 3.10 (s, 6H), 2.58 (s, 3H).

### 3) Synthesis of 6-(3-methyl-1H-pyrazol-5-yl)-4-morpholino-2-(4-(m-tolyl)-1H-pyrazol-1-yl)furo[3,2-d]pyrimidine

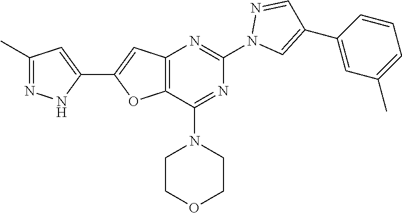 [SMILES: Cc1cccc(-c2cnn(-c3nc(N4CCOCC4)c4oc(-c5cc(C)n[nH]5)cc4n3)c2)c1]

To a solution of 5-(2-chloro-4-morpholinofuro[3,2-d]pyrimidin-6-yl)-N,N,3-trimethyl-1H-pyrazole-1-sulfonamide (90 mg, 0.21 mmol) in CH₃CN (5 mL) was added 4-(m-tolyl)-1H-pyrazole (40 mg, 0.25 mmol) and Cs₂CO₃ (138 mg, 0.42 mmol). The reaction mixture was stirred at 160° C. in a sealed tube overnight. The completion of the reaction was monitored by TLC. The reaction mixture was concentrated directly and purified by preparative TLC with a elution of 10% MeOH/DCM to provide 6-(3-methyl-1H-pyrazol-5-yl)-4-morpholino-2-(4-(m-tolyl)-1H-pyrazol-1-yl)furo[3,2-d]pyrimidine (18 mg, 0.041 mmol) as a yellow solid. LC-MS (ESI+): m/z 442 (MH⁺). ¹HNMR (300 MHz, DMSO-d₆) δ 13.52 (s, 1H), 9.00 (s, 1H), 8.22 (s, 1H), 7.61 (s, 1H), 7.57 (d, J=7.8 Hz, 1H), 7.29 (t, J=7.8 Hz, 1H), 7.15 (s, 1H), 7.08 (d, J=7.8 Hz, 1H), 6.65 (s, 1H), 4.15-4.06 (m, 4H), 3.90-3.80 (m, 4H), 2.36 (s, 3H), 2.32 (s, 3H).

Compounds 143 to 225 in Table 4 are made according to the procedures above.

TABLE 4

| | | | General | |
|---|---|---|---|---|
| Comp’d | | | Synthetic | |
| # | Name | Structure | Route | Data |
| 143 | 4-morpholino-2-[4-(m- tolyl)pyrazol-1-yl]-6- pyrimidin-4-yl- furo[3,2-d]pyrimidine | 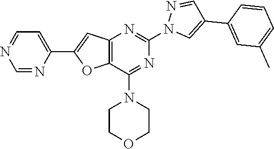 [SMILES: Cc1cccc(-c2cnn(-c3nc(N4CCOCC4)c4oc(-c5ccncn5)cc4n3)c2)c1] | 32 | LC-MS (ESI+): m/z 440 (MH⁺). ¹HNMR (300 MHz, DMSO-d₆) δ 9.34 (s, 1H), 9.05 (d, J = 4.2 Hz, 2H), 8.31-8.14 (m, 2H), 7.87-7.82 (m, 1H), 7.62-7.57 (m, 2H), 7.32- 7.27 (m, 1H), 7.10-7.05 (m, 1H), 4.27-4.14 (m, 4H), 3.95-3.85 (m, 4H), 2.36 (s, 3H). |
| 144 | 6-(3-methyl-1H- pyrazol-5-yl)-4- morpholino-2-[4-(m- tolyl)pyrazol-1- yl]furo[3,2- d]pyrimidine | 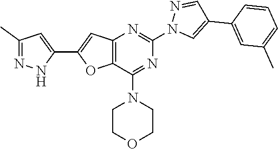 [SMILES: Cc1cccc(-c2cnn(-c3nc(N4CCOCC4)c4oc(-c5cc(C)n[nH]5)cc4n3)c2)c1] | 43 | LC-MS (ESI+): m/z 442 (MH⁺). ¹HNMR (300 MHz, DMSO-d₆) δ 13.52 (s, 1H), 9.00 (s, 1H), 8.22 (s, 1H), 7.61 (s, 1H), 7.57 (d, J = 7.8 Hz, 1H), 7.29 (t, J = 7.8 Hz, 1H), 7.15 (s, 1H), 7.08 (d, J = 7.8 Hz, 1H), 6.65 (s, 1H), 4.15-4.06 (m, 4H), 3.90-3.80 (m, 4H), 2.36 (s, 3H), 2.32 (s, 3H). |
| 145 | N,N-dimethyl-2-[3- methyl-5-[4- morpholino-2-[4-(m- tolyl)pyrazol-1- yl]furo[3,2- d]pyrimidin-6- yl]pyrazol-1- yl]ethanamine | 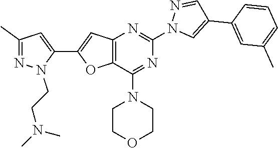 [SMILES: Cc1cccc(-c2cnn(-c3nc(N4CCOCC4)c4oc(-c5cc(C)nn5CCN(C)C)cc4n3)c2)c1] | 34 | LC-MS (ESI+): m/z 513 (MH⁺). ¹HNMR (300 MHz, CDCl₃) δ 8.72 (s, 1H), 8.08 (s, 1H), 7.61-7.41 (m, 2H), 7.35-7.27 (m, 1H), 7.12-7.06 (m, 2H), 6.45 (s, 1H), 4.48-4.43 (m, 2H), 4.15-4.10 (m, 4H), 3.93-3.88 (m, 4H), 2.85-2.80 (m, 2H), 2.41 (m, 3H), 2.32 (s, 3H), 2.29 (s, 6H). |
| 146 | N,N-dimethyl-2-[5- methyl-3-[4- morpholino-2-[4-(m- tolyl)pyrazol-1- yl]furo[3,2- d]pyrimidin-6- yl]pyrazol-1- yl]ethanamine | 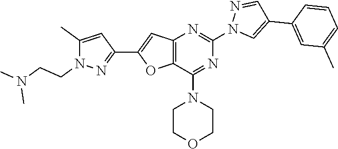 [SMILES: Cc1cccc(-c2cnn(-c3nc(N4CCOCC4)c4oc(-c5cc(C)n(CCN(C)C)n5)cc4n3)c2)c1] | 34 | LC-MS (ESI+): m/z 513 (MH⁺). ¹HNMR (300 MHz, CDCl₃) δ 8.72 (s, 1H), 8.07 (s, 1H), 7.61-7.40 (m, 2H), 7.35-7.27 (m, 1H), 7.11-7.06 (m, 2H), 6.44 (s, 1H), 4.40-4.37 (m, 2H), 4.16-4.10 (m, 4H), 3.93-3.88 (m, 4H), 3.10-2.98 (m, 2H), 2.45 (m, 3H), 2.41 (s, 9H). |
| 147 | N-cyclopropyl-4- morpholino-2-[4-(m- tolyl)pyrazol-1-yl]-7- (trifluoromethyl)furo[3, 2-d]pyrimidine-6- carboxamide | 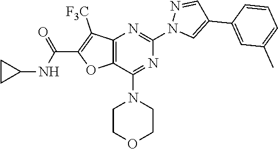 [SMILES: Cc1cccc(-c2cnn(-c3nc(N4CCOCC4)c4oc(C(=O)NC5CC5)c(C(F)(F)F)c4n3)c2)c1] | 40 | LC-MS (ESI+): m/z 513 (MH⁺). ¹HNMR (300 MHz, CDCl₃) δ 8.77 (s, 1H), 8.08 (s, 1H), 7.61-7.42 (m, 2H), 7.35-7.27 (m, 1H), 7.11-7.09 (m, 1H), 6.48 (s, 1H), 4.17-4.12 (m, 4H), 3.92-3.88 (m, 4H), 2.95-2.90 (m, 1H), 2.41 (s, 3H), 0.98-0.93 (m, 2H), 0.75-0.69 (m, 2H). |
| 148 | 4-[2-[3-(m- tolyl)pyrazol-1-yl]-6- (2-pyridyl)furo[3,2- d]pyrimidin-4- yl]morpholin-3-one | 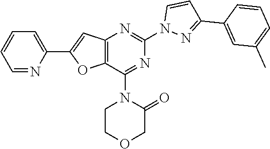 [SMILES: Cc1cccc(-c2ccn(-c3nc(N4CCOCC4=O)c4oc(-c5ccccn5)cc4n3)n2)c1] | 35 | LC-MS (ESI+): m/z 453 (MH⁺). ¹HNMR (300 MHz, CDCl₃) δ 8.75 (d, J = 3.9 Hz, 1H), 8.62 (s, 1H), 8.02 (d, J = 7.8 Hz, 1H), 7.96-7.89 (m, 2H), 7.76 (d, J = 7.5 Hz, 1H), 7.67 (s, 1H), 7.48-7.31 (m, 2H), 7.20-7.15 (m, 1H), 6.82 (s, 1H), 4.53 (s, 2H), 4.32-4.24 (m, 2H), 4.19-4.10 (m, 2H), 2.43 (s, 3H). |
| 149 | N-ethyl-2-[4-(m- tolyl)pyrazol-1-yl]-4- (3-oxomorpholin-4- yl)furo[3,2- d]pyrimidine-6- carboxamide | 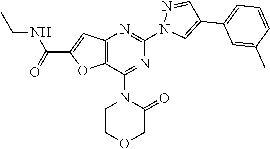 [SMILES: CCNC(=O)c1cc2nc(-n3cc(-c4cccc(C)c4)cn3)nc(N3CCOCC3=O)c2o1] | 35 | LC-MS (ESI+): m/z 447 (MH⁺). ¹HNMR (300 MHz, CDCl₃) δ 8.79 (s, 1H), 8.14 (s, 1H), 7.62 (s, 1H), 7.47-7.41 (m, 2H), 7.34-7.27 (m, 1H), 7.15-7.12 (m, 1H), 6.78 (brs, 1H), 4.51 (s, 2H), 4.34-4.31 (m, 2H), 4.21-4.16 (m, 2H), 3.59-3.49 (m, 2H), 2.42 (s, 3H), 1.32-1.27 (m, 3H). |
| 150 | N-(2- methylsulfonylethyl)-4- morpholino-2-(4- phenylpyrazol-1- yl)furo[3,2- d]pyrimidine-6- carboxamide | 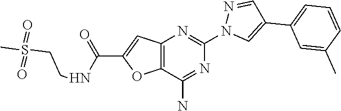 | 36 | LC-MS (ESI+): m/z 511 (MH⁺). ¹HNMR (300 MHz, CDCl₃) δ 8.70 (s, 1H), 8.07 (s, 1H), 7.67-7.62 (m, 1H), 7.53 (s, 1H), 7.48-7.40 (m, 2H), 7.35-7.26 (m, 1H), 7.12-7.09 (m, 1H), 4.15-4.05 (m, 6H), 3.92-3.85 (m, 4H), 3.34-3.07 (m, 2H), 3.04 (s, 3H), 2.42 (s, 3H). |
| 151 | N-(1-methyl-4- piperidyl)-4- morpholino-2-(3- phenylpyrazol-1- yl)furo[3,2- d]pyrimidine-6- carboxamide | 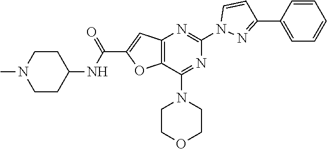 [SMILES: CN1CCC(NC(=O)c2cc3nc(-n4ccc(-c5ccccc5)n4)nc(N4CCOCC4)c3o2)CC1] | 38 | LC-MS (ESI+): m/z 488 (MH⁺). ¹HNMR (300 MHz, CDCl₃) δ 8.54 (d, J = 2.7 Hz, 1H), 7.99 (d, J = 7.2 Hz, 2H), 7.45 (s, 1H), 7.43-7.32 (m, 3H), 6.78 (d, J = 2.7 Hz, 1H), 6.25 (d, J = 8.1 Hz, 1H), 4.16-4.10 (m, 4H), 4.08-4.00 (m, 1H), 3.94-3.87 (m, 4H), 2.90-2.86 (m, 2H), 2.34 (s, 3H), 2.24-2.17 (m, 2H), 2.09-2.00 (m, 2H), 1.67-1.57 (m, 2H). |
| 152 | N-ethyl-4-morpholino- 2-(3-phenyl-1H- pyrazol-1-yl)furo[3,2- d]pyrimidine-6- carboxamide | 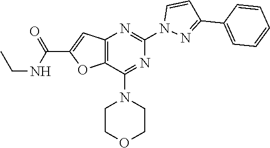 [SMILES: CCNC(=O)c1cc2nc(-n3ccc(-c4ccccc4)n3)nc(N3CCOCC3)c2o1] | 38 | LC-MS (ESI+): m/z419 (MH⁺). ¹HNMR (300 MHz, CDCl₃) δ 8.54 (d, J = 2.7 Hz, 1H), 7.99 (d, J = 7.2 Hz, 2H), 7.61 (s, 1H), 7.50-7.32 (m, 3H), 6.79 (d, J = 2.7 Hz, 1H), 6.46- 6.36 (m, 1H), 4.17-4.12 (m, 4H), 3.95-3.90 (m, 4H), 3.60-3.51 (m, 2H), 1.28 (t, J = 7.5 Hz, 3H). |
| 153 | N-cyclopropyl-4- morpholino-2-(3- phenylpyrazol-1- yl)furo[3,2- d]pyrimidine-6- carboxamide | 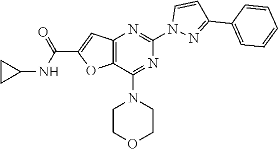 [SMILES: O=C(NC1CC1)c1cc2nc(-n3ccc(-c4ccccc4)n3)nc(N3CCOCC3)c2o1] | 38 | LC-MS (ESI+): m/z 431 (MH⁺). ¹HNMR (300 MHz, CDCl₃) δ 8.53 (d, J = 2.4 Hz, 1H), 7.99 (d, J = 7.2 Hz, 2H), 7.61 (s, 1H), 7.48-7.32 (m, 3H), 6.79 (d, J = 2.7 Hz, 1H), 6.46 (s, 1H), 4.15-4.10 (m, 4H), 3.95-3.90 (m, 4H), 2.92-2.89 (m, 1H), 0.97- 0.90 (m, 2H), 0.74-0.68 (m, 2H). |
| 154 | N-ethyl-4-morpholino- 2-(4-phenylpyrazol-1- yl)furo[3,2- d]pyrimidine-6- carboxamide | 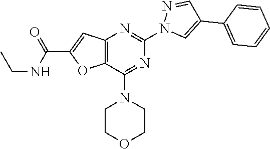 [SMILES: CCNC(=O)c1cc2nc(-n3cc(-c4ccccc4)cn3)nc(N3CCOCC3)c2o1] | 38 | LC-MS (ESI+): m/z 419 (MH⁺). ¹HNMR (300 MHz, DMSO-d₆) δ 9.05 (s, 1H), 8.91-8.89 (m, 1H), 8.25 (s, 1H), 7.79 (d, J = 6.9 Hz, 2H), 7.51 (s, 1H), 7.41 (t, J = 7.5 Hz, 2H), 7.29-7.24 (m, 1H), 4.18-4.12 (m, 4H), 3.90-3.82 (m, 4H), 3.35-3.30 (m, 2 H), 1.14 (t, J = 7.2 Hz, 3H). |
| 155 | 4-morpholino-2-(3- phenylpyrazol-1-yl)-6- (2-pyridyl)furo[3,2- d]pyrimidine | 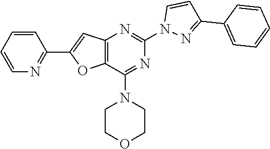 [SMILES: c1ccc(-c2ccn(-c3nc(N4CCOCC4)c4oc(-c5ccccn5)cc4n3)n2)cc1] | 33 | LC-MS (ESI+): m/z 425 (MH⁺). ¹HNMR (300 MHz, DMSO-d₆) δ 8.77-8.73 (m, 2H), 8.14 (d, J = 7.8 Hz, 1H), 8.05-7.96 (m, 3H), 7.63 (s, 1H), 7.54-7.41 (m, 3H), 7.38-7.30 (m, 1H), 7.05 (d, J = 2.4 Hz, 1H), 4.18-4.10 (m, 4H), 3.87-3.80 (m, 4H). |
| 156 | 4-morpholino-2-(3- phenylpyrazol-1-yl)-6- (3-pyridyl)furo[3,2- d]pyrimidine | 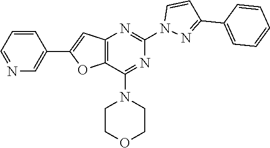 [SMILES: c1ccc(-c2ccn(-c3nc(N4CCOCC4)c4oc(-c5cccnc5)cc4n3)n2)cc1] | 33 | LC-MS (ESI+): m/z 425 (MH⁺). ¹HNMR (300 MHz, CDCl₃) δ 9.11 (s, 1H), 8.69 (d, J = 3.9 Hz, 1H), 8.57 (d, J = 2.7 Hz, 1H), 8.09 (d, J = 8.1Hz, 1H), 8.01 (d, J = 6.9 Hz, 2H), 7.45-7.32 (m, 4H), 7.30-7.26 (m, 1H), 6.79 (d, J = 2.7 Hz, 1H), 4.20- 4.14 (m, 4H), 3.97-3.92 (m, 4H). |
| 157 | N-(1-methyl-4- piperidyl)-4- morpholino-2-(4- phenylpyrazol-1- yl)furo[3,2- d]pyrimidine-6- carboxamide | 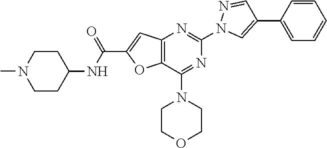 [SMILES: CN1CCC(NC(=O)c2cc3nc(-n4cc(-c5ccccc5)cn4)nc(N4CCOCC4)c3o2)CC1] | 38 | LC-MS (ESI+): m/z 488 (MH⁺). ¹HNMR (300 MHz, CDCl₃) δ 8.71 (s, 1H), 8.08 (s, 1H), 7.61 (d, J = 7.5 Hz, 2H), 7.44 (s, 1H), 7.38-7.26 (m, 3H), 6.33 (d, J = 7.8 Hz, 1H), 4.21- 4.03 (m, 5H), 3.94-3.88 (m, 4H), 2.99-2.88 (m, 2H), 2.40 (s, 3H), 2.33-2.22 (m, 2H), 2.19-2.05 (m, 2H), 1.87-1.75 (m, 2H). |
| 158 | 4-morpholino-2-(3- phenylpyrazol-1-yl)-6- (4-pyridyl)furo[3,2- d]pyrimidine | 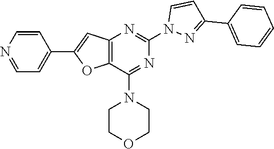 [SMILES: c1ccc(-c2ccn(-c3nc(N4CCOCC4)c4oc(-c5ccncc5)cc4n3)n2)cc1] | 33 | LC-MS (ESI+): m/z 425 (MH⁺). ¹HNMR (300 MHz, CDCl₃) δ 8.78 (d, J = 5.7 Hz, 2H), 8.56 (d, J = 2.4 Hz, 1H), 8.01 (d, J = 7.2 Hz, 2H), 7.71 (d, J = 5.7 Hz, 2H), 7.46-7.31 (m, 4H), 6.80 (d, J = 2.7 Hz, 1H), 4.19-4.13 (m, 4H), 3.97-3.92 (m, 4H). |
| 159 | 4-morpholino-N- (oxetan-3-ylmethyl)-2- (3-phenylpyrazol-1- yl)furo[3,2- d]pyrimidine-6- carboxamide | 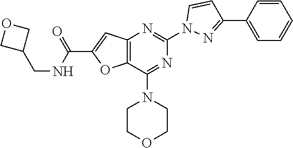 [SMILES: O=C(NCC1COC1)c1cc2nc(-n3ccc(-c4ccccc4)n3)nc(N3CCOCC3)c2o1] | 38 | LC-MS (ESI+): m/z 461 (MH⁺). ¹HNMR (300 MHz, CDCl₃) δ 8.54 (d, J = 2.7 Hz, 1H), 7.99 (d, J = 7.2 Hz, 2H), 7.45 (s, 1H), 7.41-7.31 (m, 3H), 6.79 (d, J = 2.7 Hz, 1H), 6.74- 6.64 (m, 1H), 4.87 (t, J = 6.9 Hz, 2H), 4.48 (t, J = 6.3 Hz, 2H), 4.14- 4.08 (m, 4H), 3.93-3.88 (m, 4H), 3.80 (t, J = 6.9 Hz, 2H), 3.35-3.29 (m, 1H). |
| 160 | 4-morpholino-2-(4- phenylpyrazol-1-yl)-6- (3-pyridyl)furo[3,2- d]pyrimidine | 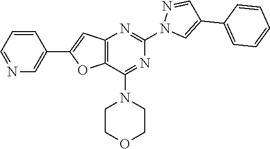 [SMILES: c1ccc(-c2cnn(-c3nc(N4CCOCC4)c4oc(-c5cccnc5)cc4n3)c2)cc1] | 33 | LC-MS (ESI+): m/z 425 (MH⁺). ¹HNMR (300 MHz, CDCl₃) δ 8.86 (s, 1H), 8.10 (d, J = 7.8 Hz, 2H), 7.63 (d, J = 7.8 Hz, 2H), 7.48-7.31 (m, 3H), 7.30-7.23 (m, 4H), 4.20- 4.13 (m, 4H), 3.97-3.91 (m, 4H). |
| 161 | 4-morpholino-2-(4- phenylpyrazol-1-yl)-6- (2-pyridyl)furo[3,2- d]pyrimidine | 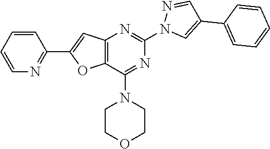 [SMILES: c1ccc(-c2cnn(-c3nc(N4CCOCC4)c4oc(-c5ccccn5)cc4n3)c2)cc1] | 33 | LC-MS (ESI+): m/z 425 (MH⁺). ¹HNMR (300 MHz, DMSO-d₆) δ 9.05 (s, 1H), 8.75 (d, J = 3.9 Hz, 1H), 8.25 (s, 1H), 8.15 (d, J = 8.1 Hz, 1H), 8.05-7.99 (m, 1H), 7.79 (d, J = 7.2 Hz, 2H), 7.54 (s, 1H), 7.53- 7.52 (m, 1H), 7.41 (t, J = 7.5 Hz, 2H), 7.29-7.25 (m, 1H), 4.20-4.10 (m, 4H), 3.90-3.80 (m, 4H). |
| 162 | 6-(3-methyl-1H- pyrazol-5-yl)-4- morpholino-2-(3- phenylpyrazol-1- yl)furo[3,2- d]pyrimidine | 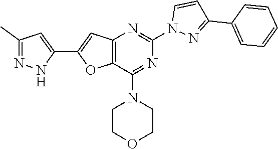 [SMILES: Cc1cc(-c2cc3nc(-n4ccc(-c5ccccc5)n4)nc(N4CCOCC4)c3o2)[nH]n1] | 43 | LC-MS (ESI+): m/z 428 (MH⁺). ¹HNMR (300 MHz, CDCl₃) δ 8.56 (d, J = 2.7 Hz, 1H), 8.00 (d, J = 6.9 Hz, 2H), 7.45-7.31 (m, 4H), 7.13 (s, 1H), 6.77 (d, J = 2.4 Hz, 1H), 6.49 (s, 1H), 4.17-4.14 (m, 4H), 3.93-3.89 (m, 4H), 2.41 (s, 3H). |
| 163 | 6-(3-methyl-1H- pyrazol-5-yl)-4- morpholino-2-(4- phenylpyrazol-1- yl)furo[3,2- d]pyrimidine | 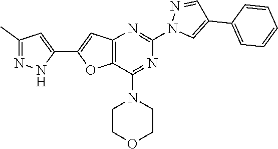 [SMILES: Cc1cc(-c2cc3nc(-n4cc(-c5ccccc5)cn4)nc(N4CCOCC4)c3o2)[nH]n1] | 43 | LC-MS (ESI+): m/z 428 (MH⁺). ¹HNMR (300 MHz, DMSO-d₆) δ 9.04 (s, 1H), 8.24 (s, 1H), 7.78 (d, J = 7.5 Hz, 2H), 7.41 (t, J = 7.5 Hz, 1H), 7.38-7.24 (m, 1H), 7.15 (s, 1H), 6.61 (s, 1H), 4.14-4.08 (m, 4H), 3.91-3.85 (m, 4H), 2.42 (s, 3H). |
| 164 | 4-morpholino-N- (oxetan-3-ylmethyl)-2- (4-phenyipyrazol-1- yl)furo[3,2- d]pyrimidine-6- carboxamide | 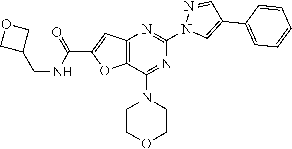 [SMILES: O=C(NCC1COC1)c1cc2nc(-n3cc(-c4ccccc4)cn3)nc(N3CCOCC3)c2o1] | 38 | LC-MS (ESI+): m/z 461 (MH⁺). ¹HNMR (300 MHz, DMSO-d₆) δ 9.05 (s, 1H), 8.99-8.97 (m, 1H), 8.26 (s, 1H), 7.79 (d, J = 7.2 Hz, 2H), 7.54 (s, 1H), 7.41 (t, J = 7.5 Hz, 2H), 7.29-7.24 (m, 1H), 4.67 (t, J = 6.0 Hz, 2H), 4.37 (t, J = 6.0 Hz, 2H), 4.14-4.08 (m, 4H), 3.93-3.85 (m, 4H), 3.60 (t, J = 6.3 Hz, 2H), 3.25- 3.20 (m, 1H). |
| 165 | N-cyclopropyl-4- morpholino-2-(4- phenylpyrazol-1- yl)furo[3,2- d]pyrimidine-6- carboxamide | 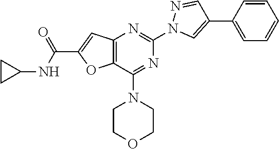 [SMILES: O=C(NC1CC1)c1cc2nc(-n3cc(-c4ccccc4)cn3)nc(N3CCOCC3)c2o1] | 38 | LC-MS (ESI+): m/z 431 (MH⁺). ¹HNMR (300 MHz, DMSO-d₆) δ 9.05 (brs, 1H), 8.90 (d, J = 3.3 Hz, 1H), 8.26 (brs, 1H), 7.79 (d, J = 7.5 Hz, 2H), 7.53 (brs, 1H), 7.41 (t, J = 7.5 Hz, 2H), 7.29-7.24 (m, 1H), 4.14-4.08 (m, 4H), 3.88-3.84 (m, 4H), 2.90-2.78 (m, 1H), 0.85-0.75 (m, 2H), 0.70-0.60 (m, 2H). |
| 166 | N-(cyclopropylmethyl)- 4-morpholino-2-(3- phenylpyrazol-1- yl)furo[3,2- d]pyrimidine-6- carboxamide | 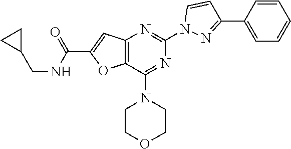 [SMILES: O=C(NCC1CC1)c1cc2nc(-n3ccc(-c4ccccc4)n3)nc(N3CCOCC3)c2o1] | 38 | LC-MS (ESI+): m/z 445 (MH⁺). ¹HNMR (300 MHz, DMSO-d₆) δ 9.05-8.99 (m, 1H), 8.72 (s, 1H), 7.97 (d, J = 7.5 Hz, 2H), 7.58 (s, 1H), 7.50-7.45 (m, 2H), 7.41-7.38 (m, 1H), 7.05 (s, 1H), 4.14-4.08 (m, 4H), 3.90-3.80 (m, 4H), 3.21 (t, J = 6.3 Hz, 2H), 1.10-1.05 (m, 1H), 0.55- 0.45 (m, 2H), 0.30-0.20 (m, 2H). |
| 167 | N-[4-(dimethylamino)- 1-methyl-butyl]-4- morpholino-2-(4- phenylpyrazol-1- yl)furo[3,2- d]pyrimidine-6- carboxamide | 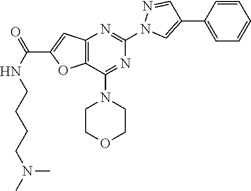 [SMILES: CN(C)CCCCNC(=O)c1cc2nc(-n3cc(-c4ccccc4)cn3)nc(N3CCOCC3)c2o1] | 38 | LC-MS (ESI+): m/z 490 (MH⁺). ¹HNMR (300 MHz, CD₃OD) δ 8.88 (s, 1H), 8.15 (s, 1H), 7.67 (d, J = 7.2 Hz, 2H), 7.45-7.40 (m, 3H), 7.29- 7.24 (m, 1H), 4.24-4.15 (m, 4H), 3.92-3.84 (m, 4H), 3.46-3.38 (m, 2H), 2.43-2.39 (m, 2H), 2.28 (s, 6H), 1.66-1.50 (m, 4H). |
| 168 | N-(3- (dimethylamino) propyl)-4-morpholino- 2-(4-phenyl-1H- pyrazol-1-yl)furo [3,2-d]pyrimidine-6- carboxamide | 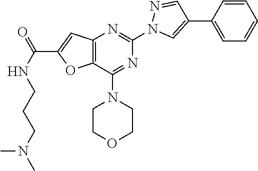 [SMILES: CN(C)CCCNC(=O)c1cc2nc(-n3cc(-c4ccccc4)cn3)nc(N3CCOCC3)c2o1] | 38 | LC-MS (ESI+): m/z 476 (MH⁺). ¹HNMR (300 MHz, CD₃OD) δ 8.88 (s, 1H), 8.16 (s, 1H), 7.68 (d, J = 7.2 Hz, 2H), 7.45-7.38 (m, 3H), 7.30- 7.25 (m, 1H), 4.25-4.15 (m, 4H), 3.93-3.85 (m, 4H), 3.46 (t, J = 6.9 Hz, 2H), 2.46 (t, J = 7.5 Hz, 2H), 2.30 (s, 6H), 1.90-1.80 (m, 2H). |
| 169 | N-(1-methyl-3- piperidyl)-4- morpholino-2-(4- phenylpyrazol-1- yl)furo[3,2- d]pyrimidine-6- carboxamide | 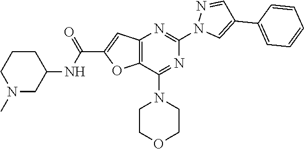 [SMILES: CN1CCCC(NC(=O)c2cc3nc(-n4cc(-c5ccccc5)cn4)nc(N4CCOCC4)c3o2)C1] | 38 | LC-MS (ESI+): m/z 488 (MH⁺). ¹HNMR (300 MHz, DMSO-d₆) δ 9.05 (s, 1H), 8.62 (brs, 1H), 8.26 (s, 1H), 7.79 (d, J = 7.5 Hz, 2H), 7.61 (s, 1H), 7.41 (t, J = 7.5 Hz, 2H), 7.29-7.24 (m, 1H), 4.15-4.10 (m, 4H), 4.05-4.00 (m, 1H), 3.89-3.81 (m, 4H), 2.95-2.70 (m, 2H), 2.25 (s, 3H), 2.11-1.70 (m, 4H), 1.59-1.42 (m, 2H). |
| 170 | N-(1-methylpyrrolidin- 3-yl)-4-morpholino-2- (4-phenylpyrazol-1- yl)furo[3,2- d]pyrimidine-6- carboxamide | 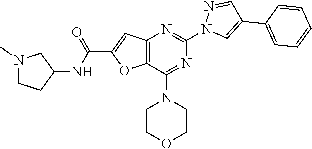 [SMILES: CN1CCC(NC(=O)c2cc3nc(-n4cc(-c5ccccc5)cn4)nc(N4CCOCC4)c3o2)C1] | 38 | LC-MS (ESI+): m/z 474 (MH⁺). ¹HNMR (300 MHz, CDCl₃) δ 8.71 (s, 1H), 8.07 (s, 1H), 7.79 (d, J = 7.5 Hz, 2H), 7.59 (s, 1H), 7.48-7.38 (m, 3H), 7.29-7.26 (m, 1H), 4.77-4.75 (m, 1H), 4.16-4.10 (m, 4H), 3.93- 3.85 (m, 4H), 3.09-3.05 (m, 1H), 2.92-2.88 (m, 1H), 2.53-2.44 (m, 2H), 2.40 (s, 3H), 2.21-2.15 (m, 1H), 1.90-1.82 (m, 1H). |
| 171 | 4-morpholino-2-(4- phenylpyrazol-1-yl)-N- tetrahydropyran-4-yl- furo[3,2-d]pyrimidine- 6-carboxamide | 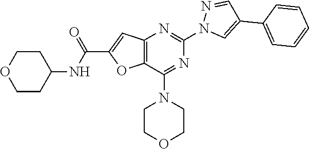 [SMILES: O=C(NC1CCOCC1)c1cc2nc(-n3cc(-c4ccccc4)cn3)nc(N3CCOCC3)c2o1] | 38 | LC-MS (ESI+): m/z 475 (MH⁺). ¹HNMR (300 MHz, DMSO-d₆) δ 9.05 (s, 1H), 8.66 (d, J = 8.1 Hz, 1H), 8.26 (s, 1H), 7.79 (d, J = 7.5 Hz, 2H), 7.57 (s, 1H), 7.41 (t, J = 7.5 Hz, 2H), 7.30-7.25 (m, 1H), 4.16-4.10 (m, 4H), 4.07-4.00 (m, 1H), 3.95-3.88 (m, 2H), 3.85-3.80 (m, 4H), 3.45-3.34 (m, 2H), 1.85- 1.79 (m, 2H), 1.75-1.60 (m, 2H). |
| 172 | 4-morpholino-2-(4- phenylpyrazol-1-yl)-N- tetrahydrofuran-3-yl- furo[3,2-d]pyrimidine- 6-carboxamide | 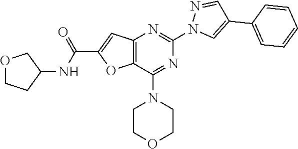 [SMILES: O=C(NC1CCOC1)c1cc2nc(-n3cc(-c4ccccc4)cn3)nc(N3CCOCC3)c2o1] | 38 | LC-MS (ESI+): m/z 461 (MH⁺). ¹HNMR (300 MHz, CDCl₃) δ 8.71 (s, 1H), 8.08 (s, 1H), 7.61 (d, J = 7.2 Hz, 2H), 7.47 (s, 1H), 7.41 (t, J = 7.2 Hz, 2H), 7.29-7.26 (m, 1H), 6.53 (d, J = 7.2 Hz, 1H), 4.79-4.75 (m, 1H), 4.20-4.15 (m, 4H), 4.10-4.00 (m, 1H), 3.93-3.75 (m, 7H), 2.52-2.38 (m, 1H), 2.10-1.90 (m, 1H). |
| 173 | 6-(3-methyl-1H- pyrazol-5-yl)-4- morpholino-2-[3-(m- tolyl)pyrazol-1- yl]furo[3,2- d]pyrimidine | 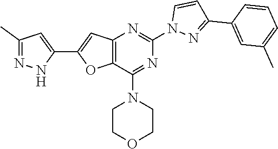 [SMILES: Cc1cccc(-c2ccn(-c3nc(N4CCOCC4)c4oc(-c5cc(C)n[nH]5)cc4n3)n2)c1] | 43 | LC-MS (ESI+): m/z 442 (MH⁺). ¹HNMR (300 MHz, CDCl₃) δ 8.55 (d, J = 2.7 Hz, 1H), 7.90 (s, 1H), 7.77 (d, J = 7.5 Hz, 1H), 7.33-7.26 (m, 2H), 7.19-7.12 (m, 2H), 6.77 (d, J = 2.7 Hz, 1H), 6.50 (s, 1H), 4.20- 4.15 (m, 4H), 3.93-3.85 (m, 4H), 2.42 (s, 6H). |
| 174 | 4-morpholino-N- (oxetan-3-yl)-2-(4- phenylpyrazol-1- yl)furo[3,2- d]pyrimidine-6- carboxamide | 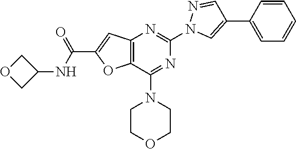 [SMILES: O=C(NC1COC1)c1cc2nc(-n3cc(-c4ccccc4)cn3)nc(N3CCOCC3)c2o1] | 38 | LC-MS (ESI+): m/z 447 (MH⁺). ¹HNMR (300 MHz, DMSO-d₆) δ 9.04 (s, 1H), 8.26 (s, 1H), 7.78 (d, J = 7.2 Hz, 2H), 7.53 (s, 1H), 7.41 (t, J = 7.5 Hz, 2H), 7.30-7.25 (m, 1H), 4.95-4.91 (m, 1H), 4.54-4.49 (m, 1H), 4.40-4.32 (m, 2H), 4.16-4.05 (m, 4H), 3.85-3.80 (m, 4H), 3.62- 3.55 (m, 2H). |
| 175 | 4-morpholino-N- (oxetan-3-yl)-2-(3- phenylpyrazol-1- yl)furo[3,2- d]pyrimidine-6- carboxamide | 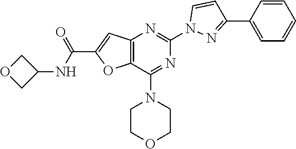 [SMILES: O=C(NC1COC1)c1cc2nc(-n3ccc(-c4ccccc4)n3)nc(N3CCOCC3)c2o1] | 38 | LC-MS (ESI+): m/z 447 (MH⁺). ¹HNMR (300 MHz, DMSO-d₆) δ 8.72 (d, J = 2.7 Hz, 1H), 7.95 (d, J = 7.5 Hz, 2H), 7.58 (s, 1H), 7.47 (t, J = 12 Hz, 2H), 7.40-7.35 (m, 1H), 7.05 (d, J = 2.4 Hz, 1H), 4.93 (t, J = 5.7 Hz, 1H), 4.54-4.41 (m, 1H), 4.40- 4.32 (m, 2H), 4.16-4.05 (m, 4H), 3.85-3.80 (m, 4H), 3.82-3.65 (m, 2H). |
| 176 | N-(1-methylazepan-4- yl)-4-morpholino-2-(4- phenylpyrazol-1- yl)furo[3,2- d]pyrimidine-6- carboxamide | 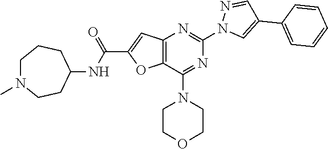 | 38 | LC-MS (ESI+): m/z 502 (MH⁺). ¹HNMR (300 MHz, CDCl₃) δ 8.72 (s, 1H), 8.08 (s, 1H), 7.85-7.75 (m, 1H), 7.61-7.59 (m, 2H), 7.48-7.38 (m, 3H), 7.30-7.27 (m, 1H), 4.70- 4.63 (m, 1H), 4.18-4.12 (m, 4H), 3.92-3.87 (m, 4H), 3.03-2.80 (m, 2H), 2.75-2.55 (m, 2H), 2.50 (s, 3H), 2.20-1.90 (m, 4H), 1.85-1.70 (m, 2H). |
| 177 | N,N-dimethyl-2-[5- methyl-3-[4- morpholino-2-(3- phenylpyrazol-1- yl)furo[3,2- d]pyrimidin-6- yl]pyrazol-1- yl]ethanamine | 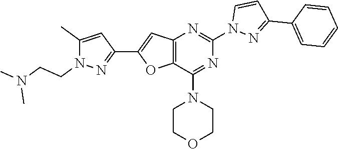 [SMILES: Cc1cc(-c2cc3nc(-n4ccc(-c5ccccc5)n4)nc(N4CCOCC4)c3o2)nn1CCN(C)C] | 34 | LC-MS (ESI+): m/z 499 (MH⁺). ¹HNMR (300 MHz, CDCl₃) δ 8.56 (d, J = 2.7 Hz, 1H), 8.01 (d, J = 6.9 Hz, 2H), 7.48-7.32 (m, 3H), 7.10 (s, 1H), 6.77 (d, J = 2.7 Hz, 1H), 6.43 (s, 1H), 4.31-4.27 (m, 2H), 4.16-4.10 (m, 4H), 3.93-3.87 (m, 4H), 2.95- 2.85 (m, 2H), 2.39 (s, 3H), 2.37 (s, 6H). |
| 178 | N,N-dimethyl-2-[5- methyl-3-[4- morpholino-2-[3-(m- tolyl)pyrazol-1- yl]furo[3,2- d]pyrimidin-6- yl]pyrazol-1- yl]ethanamine | 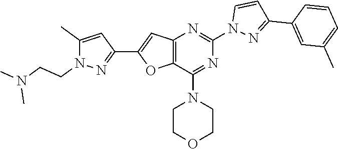 [SMILES: Cc1cccc(-c2ccn(-c3nc(N4CCOCC4)c4oc(-c5cc(C)n(CCN(C)C)n5)cc4n3)n2)c1] | 34 | LC-MS (ESI+): m/z 513 (MH⁺). ¹HNMR (300 MHz, CDCl₃) δ 8.55 (d, J = 2.4 Hz, 1H), 7.90 (s, 1H), 7.76 (d, J = 7.8 Hz, 1H), 7.33-7.26 (m, 1H), 7.16-7.12 (m, 2H), 6.77 (d, J = 1.8 Hz, 1H), 6.46 (s, 1H), 4.77- 4.70 (m, 2H), 4.19-4.10 (m, 4H), 3.93-3.85 (m, 4H), 3.70-3.60 (m, 2H), 2.82 (s, 6H), 2.52 (s, 3H), 2.42 (s, 3H). |
| 179 | 7-methyl-4- morpholino-2-(3- phenylpyrazol-1-yl)-6- (2-pyridyl)furo[3,2- d]pyrimidine | 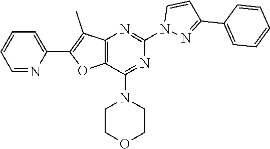 [SMILES: Cc1c(-c2ccccn2)oc2c(N3CCOCC3)nc(-n3ccc(-c4ccccc4)n3)nc12] | 39 | LC-MS (ESI+): m/z 439 (MH⁺). ¹HNMR (300 MHz, CDCl₃) δ 8.76 (d, J = 1.5 Hz, 1H), 8.62 (d, J = 2.7 Hz, 1H), 8.02 (d, J = 7.2 Hz, 2H), 7.83-7.81 (m, 2H), 7.46-7.41 (m, 2H), 7.37-7.27 (m, 2H), 6.79 (d, J = 2.7 Hz, 1H), 4.21-4.15 (m, 4H), 3.96-3.90 (m, 4H), 2.77 (s, 3H). |
| 180 | 7-methyl-4- morpholino-2-(3- phenylpyrazol-1-yl)-6- (3-pyridyl)furo[3,2- d]pyrimidine | 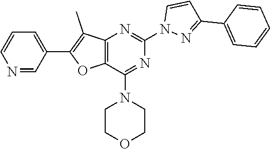 [SMILES: Cc1c(-c2cccnc2)oc2c(N3CCOCC3)nc(-n3ccc(-c4ccccc4)n3)nc12] | 39 | LC-MS (ESI+): m/z 439 (MH⁺). ¹HNMR (300 MHz, CDCl₃) δ 9.10 (s, 1H), 8.67 (d, J = 3.9 Hz, 1H), 8.61 (d, J = 2.7 Hz, 1H), 8.11 (d, J = 8.1Hz, 1H), 8.01 (d, J = 7.2 Hz, 2H), 7.46-7.35 (m, 4H), 6.79 (d, J = 2.7 Hz, 1H), 4.20-4.15 (m, 4H), 3.95- 3.90 (m, 4H), 2.61(s, 3H). |
| 181 | 7-methyl-4- morpholino-2-(3- phenylpyrazol-1-yl)-6- (4-pyridyl)furo[3,2- d]pyrimidine | 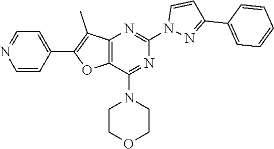 [SMILES: Cc1c(-c2ccncc2)oc2c(N3CCOCC3)nc(-n3ccc(-c4ccccc4)n3)nc12] | 39 | LC-MS (ESI+): m/z 439 (MH⁺). ¹HNMR (300 MHz, CDCl₃) δ 8.77 (dd, J = 1.5, 4.8 Hz, 2H), 8.61 (d, J = 2.4 Hz, 1H), 8.01 (d, J = 7.2 Hz, 2H), 7.69-7.61 (m, 2H), 7.46-7.32 (m, 3H), 6.79 (d, J = 2.7 Hz, 1H), 4.23- 4.17 (m, 4H), 3.97-3.90 (m, 4H), 2.65 (s, 3H). |
| 182 | 7-methyl-6-(3-methyl- 1H-pyrazol-5-yl)-4- morpholino-2-(3- phenylpyrazol-1- yl)furo[3,2- d]pyrimidine | 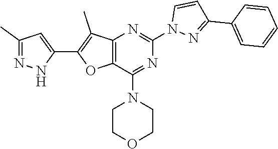 [SMILES: Cc1cc(-c2oc3c(N4CCOCC4)nc(-n4ccc(-c5ccccc5)n4)nc3c2C)[nH]n1] | 43 and 39 | LC-MS (ESI+): m/z 442 (MH⁺). ¹HNMR (300 MHz, CDCl₃) δ 8.60 (d, J = 2.7 Hz, 1H), 8.01 (d, J = 7.2 Hz, 2H), 7.46-7.32 (m, 4H), 6.79 (d, J = 2.7 Hz, 1H), 6.45 (s, 1H), 4.20- 4.15 (m, 4H), 3.93-3.88 (m, 4H), 2.56 (s, 3H), 2.42 (s, 3H). |
| 183 | N,N-dimethyl-2-[5- methyl-3-[4- morpholino-2-(4- phenylpyrazol-1- yl)furo[3,2- d]pyrimidin-6- yl]pyrazol-1- yl]ethanamine | 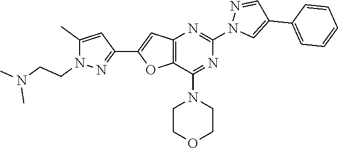 [SMILES: Cc1cc(-c2cc3nc(-n4cc(-c5ccccc5)cn4)nc(N4CCOCC4)c3o2)nn1CCN(C)C] | 34 | LC-MS (ESI+): m/z 499 (MH⁺). ¹HNMR (300 MHz, DMSO-d₆) δ 9.05 (brs, 1H), 8.21 (brs, 1H), 7.79 (d, J = 7.8 Hz, 2H), 7.43-7.38 (m, 2H), 7.29-7.24 (m, 1H), 7.17 (brs, 1H), 6.72 (s, 1H), 4.42-4.30 (m, 2H), 4.13-4.08 (m, 4H), 3.89-3.82 (m, 4H), 3.05-2.95 (m, 2H), 2.50 (s, 6H), 2.42 (s, 3H). |
| 184 | N-cyclopropyl-7- methyl-4-morpholino- 2-(3-phenylpyrazol-1- yl)furo[3,2- d]pyrimidine-6- carboxamide | 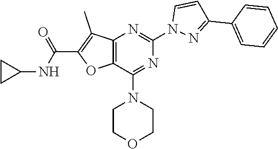 [SMILES: Cc1c(C(=O)NC2CC2)oc2c(N3CCOCC3)nc(-n3ccc(-c4ccccc4)n3)nc12] | 39 and 40 | LC-MS (ESI+): m/z 445 (MH⁺). ¹HNMR (300 MHz, CDCl₃) δ 8.58 (d, J = 2.7 Hz, 1H), 8.00 (d, J = 7.2 Hz, 2H), 7.46-7.32 (m, 3H), 6.79 (d, J = 2.7 Hz, 1H), 6.40 (s, 1H), 4.15- 4.10 (m, 4H), 3.95-3.90 (m, 4H), 2.89-2.86 (m, 1H), 2.67 (s, 3H), 0.95-0.90 (m, 2H), 0.72-0.69 (m, 2H). |
| 185 | 2-[3-(3- chlorophenyl)pyrazol- 1-yl]-4-morpholino-6- (2-pyridyl)furo[3,2- d]pyrimidine | 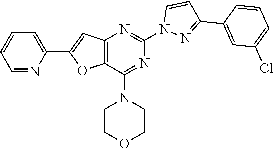 [SMILES: Clc1cccc(-c2ccn(-c3nc(N4CCOCC4)c4oc(-c5ccccn5)cc4n3)n2)c1] | 33 and 37 | LC-MS (ESI+): m/z 459/461 (MH⁺). ¹HNMR (300 MHz, CDCl₃) δ 8.74 (d, J = 4.8 Hz, 1H), 8.58 (d, J = 2.4 Hz, 1H), 8.04 (s, 1H), 7.90-7.83 (m, 3H), 7.56 (s, 1H), 7.39-7.28 (m, 3H), 6.77 (d, J = 2.4 Hz, 1H), 4.21-4.15 (m, 4H), 3.98-3.91 (m, 4H). |
| 186 | 2-[3-(3- bromophenyl)pyrazol- 1-yl]-4-morpholino-6- (2-pyridyl)furo[3,2- d]pyrimidine | 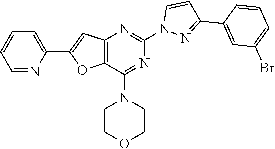 [SMILES: Brc1cccc(-c2ccn(-c3nc(N4CCOCC4)c4oc(-c5ccccn5)cc4n3)n2)c1] | 33 and 37 | LC-MS (ESI+): m/z 503/505 (MH⁺). ¹HNMR (300 MHz, CDCl₃) δ 8.74 (d, J = 4.5 Hz, 1H), 8.58 (s, 1H), 8.19 (s, 1H), 7.94-7.89 (m, 1H), 7.84-7.83 (m, 2H), 7.63 (s, 1H), 7.56-7.46 (m, 1H), 7.35-7.32 (m, 2H), 6.77 (s, 1H), 4.21-4.15 (m, 4H), 3.98-3.91 (m, 4H). |
| 187 | 2-[3-(6-methyl-2- pyridyl)pyrazol-1-yl]- 4-morpholino-6-(2- pyridyl)furo[3,2- d]pyrimidine | 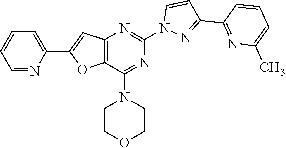 [SMILES: Cc1cccc(-c2ccn(-c3nc(N4CCOCC4)c4oc(-c5ccccn5)cc4n3)n2)n1] | 33 and 37 | LC-MS (ESI+): m/z 440 (MH⁺). ¹HNMR (300 MHz, CDCl₃) δ 8.74 (d, J = 4.8 Hz, 1H), 8.60 (d, J = 2.7 Hz, 1H), 8.14 (d, J = 7.8 Hz, 1H), 7.85-7.83 (m, 2H), 7.68-7.58 (m, 1H), 7.56 (s, 1H), 7.35-7.32 (m, 1H), 7.18 (d, J = 2.7 Hz, 1H), 7.12-7.10 (m, 1H), 4.21-4.15 (m, 4H), 3.99- 3.92 (m, 4H), 2.63 (s, 3H). |
| 188 | 2-[3-(2-methyl-4- pyridyl)pyrazol-1-yl]- 4-morpholino-6-(2- pyridyl)furo[3,2- d]pyrimidine | 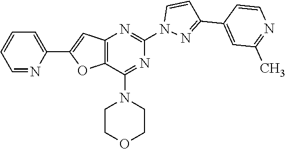 [SMILES: Cc1cc(-c2ccn(-c3nc(N4CCOCC4)c4oc(-c5ccccn5)cc4n3)n2)ccn1] | 33 and 37 | LC-MS (ESI+): m/z 440 (MH⁺). ¹HNMR (300 MHz, CDCl₃) δ 8.74 (d, J = 4.5 Hz, 1H), 8.61 (d, J = 2.4 Hz, 1H), 8.56 (brs, 1H), 7.85-7.80 (m, 3H), 7.67 (brs, 1H), 7.56 (s, 1H), 7.35-7.28 (m, 1H), 6.84 (d, J = 2.7 Hz, 1H), 4.22-4.15 (m, 4H), 3.98- 3.89 (m, 4H), 2.64 (s, 3H). |
| 189 | 6-(1-methylpyrazol-3- yl)-4-morpholino-2-[3- (m-tolyl)pyrazol-1- yl]furo[3,2- d]pyrimidine | 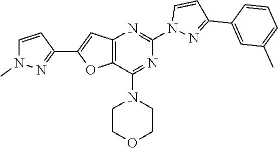 [SMILES: Cc1cccc(-c2ccn(-c3nc(N4CCOCC4)c4oc(-c5ccn(C)n5)cc4n3)n2)c1] | 43 | LC-MS (ESI+): m/z 442 (MH⁺). ¹HNMR (300 MHz, CDCl₃) δ 8.56 (s, 1H), 7.91 (s, 1H), 7.77 (d, J = 7.8 Hz, 1H), 7.41 (s, 1H), 7.33-7.28 (m, 1H), 7.17-7.10 (m, 2H), 6.77 (s, 1H), 6.66 (d, J = 2.1 Hz, 1H), 4.21-4.15 (m, 4H), 4.02 (s, 3H), 3.95-3.89 (m, 4H), 2.42 (s, 3H). |
| 190 | 6-(1-methylpyrazol-3- yl)-4-morpholino-2-(4- phenylpyrazol-1- yl)furo[3,2- d]pyrimidine | 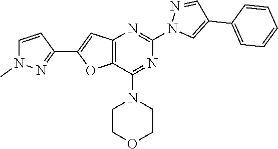 [SMILES: Cn1ccc(-c2cc3nc(-n4cc(-c5ccccc5)cn4)nc(N4CCOCC4)c3o2)n1] | 43 | LC-MS (ESI+): m/z 428 (MH⁺). ¹HNMR (300 MHz, CDCl₃) δ 8.75 (s, 1H), 8.07 (s, 1H), 7.62 (d, J = 7.2 Hz, 2H), 7.46-7.38 (m, 3H), 7.29- 7.28 (m, 1H), 7.11 (s, 1H), 6.66 (d, J = 2.1 Hz, 1H), 4.21-4.15 (m, 4H), 4.02 (s, 3H), 3.96-3.89 (m, 4H). |
| 191 | 6-(1-methylpyrazol-3- yl)-4-morpholino-2-(3- phenylpyrazol-1- yl)furo[3,2- d]pyrimidine | 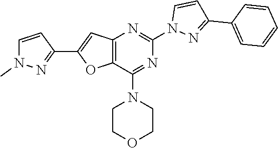 [SMILES: Cn1ccc(-c2cc3nc(-n4ccc(-c5ccccc5)n4)nc(N4CCOCC4)c3o2)n1] | 43 | LC-MS (ESI+): m/z 428 (MH). ¹HNMR (300 MHz, CDCl₃) δ 8.56 (d, J = 2.7 Hz, 1H), 8.02 (d, J = 7.2 Hz, 2H), 7.46-7.38 (m, 3H), 7.29- 7.28 (m, 1H), 7.18 (s, 1H), 6.78 (d, J = 2.7 Hz, 1H), 6.66 (d, J = 2.1 Hz, 1H), 4.19-4.10 (m, 4H), 4.02 (s, 3H), 3.96-3.89 (m, 4H). |
| 192 | 2-[4-(3- chlorophenyl)pyrazol- 1-yl]-6-(1- methylpyrazol-3-yl)-4- morpholino-furo[3,2- d]pyrimidine | 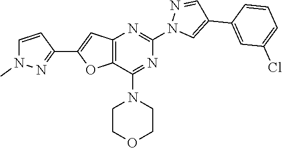 [SMILES: Cn1ccc(-c2cc3nc(-n4cc(-c5cccc(Cl)c5)cn4)nc(N4CCOCC4)c3o2)n1] | 43 and 37 | LC-MS (ESI+): m/z 462/464 (MH⁺). ¹HNMR (300 MHz, CDCl₃) δ 8.75 (s, 1H), 8.06 (s, 1H), 7.62 (s, 1H), 7.50-7.40 (m, 2H), 7.26-7.20 (m, 2H), 7.11 (s, 1H), 6.66 (d, J = 2.4 Hz, 1H), 4.21-4.15 (m, 4H), 4.02 (s, 3H), 3.95-3.89 (m, 4H). |
| 193 | 2-[3-(3- chlorophenyl)pyrazol- 1-yl]-6-(1- methylpyrazol-3-yl)-4- morpholino-furo[3,2- d]pyrimidine | 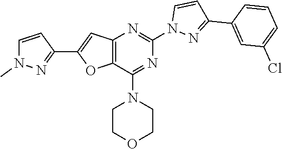 [SMILES: Cn1ccc(-c2cc3nc(-n4ccc(-c5cccc(Cl)c5)n4)nc(N4CCOCC4)c3o2)n1] | 43 and 37 | LC-MS (ESI+): m/z 462/464 (MH⁺). ¹HNMR (300 MHz, CDCl₃) δ 8.61 (d, J = 2.7 Hz, 1H), 8.09 (s, 1H), 7.90-7.87 (m, 1H), 7.45 (d, J = 1.8 Hz, 1H), 7.29-7.28 (m, 2H), 7.15 (s, 1H), 6.76 (s, 1H), 6.66 (d, J = 2.1 Hz, 1H), 4.19-4.10 (m, 4H), 4.02 (s, 3H), 3.96-3.89 (m, 4H). |
| 194 | 2-(4-bromopyrazol-1- yl)-6-(1-methylpyrazol- 3-yl)-4-morpholino- furo[3,2-d]pyrimidine | 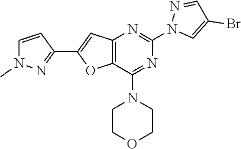 [SMILES: Cn1ccc(-c2cc3nc(-n4cc(Br)cn4)nc(N4CCOCC4)c3o2)n1] | 43 | LC-MS (ESI+): m/z 430/432 (MH⁺). ¹HNMR (300 MHz, DMSO-d₆) δ 8.92 (s, 1H), 7.95-7.89 (m, 2H), 7.18 (s, 1H), 6.89 (d, J = 1.8 Hz, 1H), 4.12-4.06 (m, 4H), 3.95 (s, 3H), 3.85-3.77 (m, 4H). |
| 195 | 2-(3-bromopyrazol-1- yl)-6-(1-methylpyrazol- 3-yl)-4-morpholino- furo[3,2-d]pyrimidine | 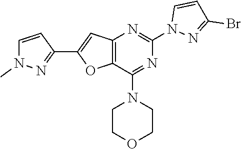 [SMILES: Cn1ccc(-c2cc3nc(-n4ccc(Br)n4)nc(N4CCOCC4)c3o2)n1] | 43 | LC-MS (ESI+): m/z 430/432 (MH⁺). ¹HNMR (300 MHz, CDCl₃) δ 8.52 (s, 1H), 7.46 (s, 1H), 7.11 (s, 1H), 6.56 (s, 1H), 6.45 (s, 1H), 4.18-4.06 (m, 4H), 4.02 (s, 3H), 3.92-3.85 (m, 4H). |
| 196 | N-[2-[5-methyl-3-[4- morpholino-2-[4-(m- tolyl)pyrazol-1- yl]furo[3,2- d]pyrimidin-6- yl]pyrazol-1- yl]ethyl]prop-2- enamide | 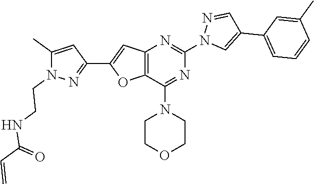 [SMILES: C=CC(=O)NCCn1nc(-c2cc3nc(-n4cc(-c5cccc(C)c5)cn4)nc(N4CCOCC4)c3o2)cc1C] | 34 | LC-MS (ESI+): m/z 539 (MH⁺). ¹HNMR (300 MHz, DMSO-d₆) δ 9.00 (s, 1H), 8.35-8.30 (m, 1H), 8.22 (s, 1H), 7.62-7.51 (m, 2H), 7.29-7.20 (m, 1H), 7.16 (s, 1H), 7.09-7.05 (m, 1H), 6.71 (s, 1H), 6.18-6.05 (m, 2H), 5.63-5.60 (m, 1H), 4.26-4.21 (m, 2H), 4.11-4.05 (m, 4H), 3.85-3.79 (m, 4H), 3.55-3.45 (m, 2H), 2.36 (s, 3H), 2.29 (s, 3H). |
| 197 | N-[2-[5-methyl-3-[4- morpholino-2-[4-(m- tolyl)pyrazol-1- yl]furo[3,2- d]pyrimidin-6- yl]pyrazol-1- yl]ethyl]acetamide | 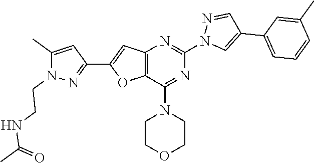 [SMILES: CC(=O)NCCn1nc(-c2cc3nc(-n4cc(-c5cccc(C)c5)cn4)nc(N4CCOCC4)c3o2)cc1C] | 34 | LC-MS (ESI+): m/z 527 (MH⁺). ¹HNMR (300 MHz, DMSO-d₆) δ 9.00 (s, 1H), 8.22 (s, 1H), 8.04-8.00 (m, 1H), 7.62-7.51 (m, 2H), 7.31- 7.27 (m, 1H), 7.16 (s, 1H), 7.09-7.05 (m, 1H), 6.71 (s, 1H), 4.22-4.16 (m, 2H), 4.14-4.05 (m, 4H), 3.85-3.80 (m, 4H), 3.45-3.35 (m, 2H), 2.36 (s, 3H), 2.29 (s, 3H), 1.80 (s, 3H). |
| 198 | 2-chloro-N-[2-[5- methyl-3-[4- morpholino-2-[4-(m- tolyl)pyrazol-1- yl]furo[3,2- d]pyrimidin-6- yl]pyrazol-1- yl]ethyl]acetamide | 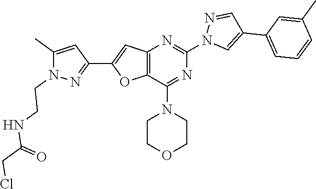 [SMILES: Cc1cccc(-c2cnn(-c3nc(N4CCOCC4)c4oc(-c5cc(C)n(CCNC(=O)CCl)n5)cc4n3)c2)c1] | 34 | LC-MS (ESI+): m/z 561/563 (MH⁺). ¹HNMR (300 MHz, DMSO-d₆) δ 9.01 (s, 1H), 8.44-8.42 (m, 1H), 8.23 (s, 1H), 7.62-7.51 (m, 2H), 7.32-7.27 (m, 1H), 7.16 (s, 1H), 7.09-7.05 (m, 1H), 6.71 (s, 1H), 4.25-4.16 (m, 2H), 4.14-4.05 (m, 4H), 4.02 (s, 2H), 3.88-3.80 (m, 4H), 3.53-3.45 (m, 2H), 2.36 (s, 3H), 2.33 (s, 3H). |
| 199 | 2-[3-(3- methoxyphenyl) pyrazol-1-yl]-4- morpholino-6- (2-pyridyl)furo[3,2- d]pyrimidine | 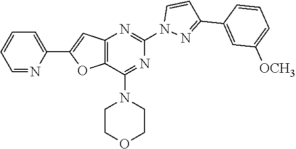 [SMILES: COc1cccc(-c2ccn(-c3nc(N4CCOCC4)c4oc(-c5ccccn5)cc4n3)n2)c1] | 33 and 37 | LC-MS (ESI+): m/z 455 (MH⁺). ¹HNMR (300 MHz, CDCl₃) δ 8.74 (d, J = 4.5 Hz, 1H), 8.58 (brs, 1H), 7.85-7.83 (m, 2H), 7.61-7.45 (m, 3H), 7.37-7.26 (m, 2H), 6.92-6.90 (m, 1H), 6.78 (s, 1H), 4.23-4.14 (m, 4H), 4.00-3.90 (m, 4H), 3.85 (s, 3H). |
| 200 | 2-[3-(5-methyl-3- pyridyl)pyrazol-1-yl]- 4-morpholino-6-(2- pyridyl)furo[3,2- d]pyrimidine | 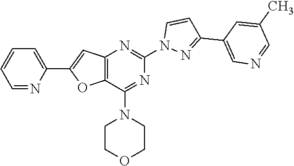 [SMILES: Cc1cncc(-c2ccn(-c3nc(N4CCOCC4)c4oc(-c5ccccn5)cc4n3)n2)c1] | 33 and 37 | LC-MS (ESI+): m/z 440 (MH⁺). ¹HNMR (300 MHz, CDCl₃) δ 8.94 (s, 1H), 8.74 (d, J = 4.8 Hz, 1H), 8.61 (d, J = 2.7 Hz, 1H), 8.42 (brs, 1H), 8.30 (s, 1H), 7.85-7.83 (m, 2H), 7.56 (s, 1H), 7.39-7.30 (m, 1H), 6.83 (d, J = 2.4 Hz, 1H), 4.25-4.17 (m, 4H), 4.00-3.90 (m, 4H), 2.42 (s, 3H). |
| 201 | 6-(1-methylpyrazol-3- yl)-4-morpholino-2-[4- (m-tolyl)pyrazol-1- yl]furo[3,2- d]pyrimidine | 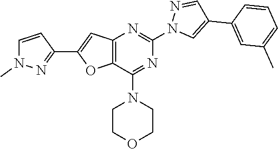 [SMILES: Cc1cccc(-c2cnn(-c3nc(N4CCOCC4)c4oc(-c5ccn(C)n5)cc4n3)c2)c1] | 43 | LC-MS (ESI+): m/z 442 (MH⁺). ¹HNMR (300 MHz, DMSO-d₆) δ 9.00 (brs, 1H), 8.23 (brs, 1H), 7.90 (d, J = 2.7 Hz, 1H), 7.62-7.51 (m, 2H), 7.29 (t, J = 7.5 Hz, 1H), 7.20 (brs, 1H), 7.08 (d, J = 7.2 Hz, 1H), 6.90 (d, J = 1.8 Hz, 1H), 4.15-4.05 (m, 4H), 4.02 (s, 3H), 3.85-3.79 (m, 4H), 2.36 (s, 3H). |
| 202 | 1-[2-[5-methyl-3-[4- morpholino-2-[4-(m- tolyl)pyrazol-1- yl]furo[3,2- d]pyrimidin-6- yl]pyrazol-1- yl]ethyl]pyrrole- 2,5-dione | 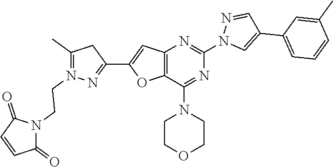 | 34 | LC-MS (ESI+): m/z 565 (MH⁺). ¹HNMR (300 MHz, DMSO-d₆) δ 9.03 (s, 1H), 8.22 (s, 1H), 7.62-7.51 (m, 2H), 7.32-7.27 (m, 1H), 7.09- 7.02 (m, 4H), 6.70 (s, 1H), 4.33-4.29 (m, 2H), 4.15-4.05 (m, 4H), 3.85- 3.75 (m, 6H), 2.36 (s, 3H), 2.33 (s, 3H). |
| 203 | 4-morpholino-2-[3-(m- tolyl)pyrazol-1-yl]-6- thiazol-2-yl-furo[3,2- d]pyrimidine | 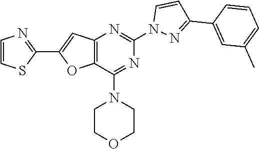 [SMILES: Cc1cccc(-c2ccn(-c3nc(N4CCOCC4)c4oc(-c5nccs5)cc4n3)n2)c1] | 41 | LC-MS (ESI+): m/z 445 (MH⁺). ¹HNMR (300 MHz, CDCl₃) δ 8.55 (d, J = 2.7 Hz, 1H), 8.01 (d, J = 3.0 Hz, 1H), 7.91 (s, 1H), 7.76 (d, J = 7.8 Hz, 1H), 7.52 (d, J = 3.0 Hz, 1H), 7.48 (s, 1H), 7.32 (t, J = 7.5 Hz, 1H), 7.16 (d, J = 7.8 Hz, 1H), 6.78 (d, J = 2.7 Hz, 1H), 4.19-4.15 (m, 4H), 3.96-3.92 (m, 4H), 2.42 (s, 3H). |
| 204 | 6-(3-methylisoxazol-5- yl)-4-morpholino-2-[3- (m-tolyl)pyrazol-1- yl]furo[3,2- d]pyrimidine | 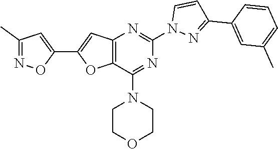 [SMILES: Cc1cccc(-c2ccn(-c3nc(N4CCOCC4)c4oc(-c5cc(C)no5)cc4n3)n2)c1] | 41 | LC-MS (ESI+): m/z 443 (MH⁺). ¹HNMR (300 MHz, CDCl₃) δ 8.54 (d, J = 2.7 Hz, 1H), 7.90 (s, 1H), 7.76 (d, J = 7.5 Hz, 1H), 7.39-7.26 (m, 2H), 7.17 (d, J = 7.8 Hz, 1H), 6.78 (d, J = 2.7 Hz, 1H), 6.58 (s, 1H), 4.17-4.13 (m, 4H), 3.95-3.91 (m, 4H), 2.42 (s, 6H). |
| 205 | 6-(1-methylpyrazol-4- yl)-4-morpholino-2-[3- (m-tolyl)pyrazol-1- yl]furo[3,2- d]pyrimidine | 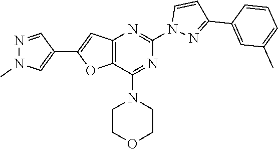 [SMILES: Cc1cccc(-c2ccn(-c3nc(N4CCOCC4)c4oc(-c5cnn(C)c5)cc4n3)n2)c1] | 41 | LC-MS (ESI+): m/z 442 (MH⁺). ¹HNMR (300 MHz, CDCl₃) δ 8.54 (d, J = 2.7 Hz, 1H), 7.90 (s, 1H), 7.87 (s, 1H), 7.81-7.76 (m, 2H), 7.31 (t, J = 7.5 Hz, 1H), 7.16 (d, J = 7.2 Hz, 1H), 6.84 (s, 1H), 6.77 (d, J = 2.4 Hz, 1H), 4.13-4.09 (m, 4H), 4.00 (s, 3H), 3.94-3.90 (m, 4H), 2.42 (s, 3H). |
| 206 | 6-(3-methylisoxazol-5- yl)-4-morpholino-2-(3- phenylpyrazol-1- yl)furo[3,2- d]pyrimidine | 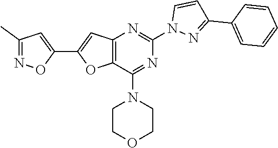 [SMILES: Cc1cc(-c2cc3nc(-n4ccc(-c5ccccc5)n4)nc(N4CCOCC4)c3o2)on1] | 41 | LC-MS (ESI+): m/z 429 (MH⁺). ¹HNMR (300 MHz, CDCl₃) δ 8.55 (d, J = 2.7 Hz, 1H), 8.01 (d, J = 6.9 Hz, 2H), 7.46-7.41 (m, 2H), 7.38- 7.26 (m, 2H), 6.80 (d, J = 2.1 Hz, 1H), 6.59 (s, 1H), 4.17-4.13 (m, 4H), 3.95-3.91 (m, 4H), 2.42 (s, 3H). |
| 207 | 4-morpholino-2-(3- phenylpyrazol-1-yl)-6- thiazol-2-yl-furo[3,2- d]pyrimidine | 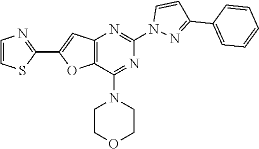 [SMILES: c1ccc(-c2ccn(-c3nc(N4CCOCC4)c4oc(-c5nccs5)cc4n3)n2)cc1] | 41 | LC-MS (ESI+): m/z 431 (MH⁺). ¹HNMR (300 MHz, CDCl₃) δ 8.56 (d, J = 2.4 Hz, 1H), 8.02-8.00 (m, 3H), 7.53-7.35 (m, 5H), 6.80 (d, J = 2.7 Hz, 1H), 4.19-4.15 (m, 4H), 3.96-3.92 (m, 4H). |
| 208 | 6-(1-methylpyrazol-4- yl)-4-morpholino-2-(3- phenylpyrazol-1- yl)furo[3,2- d]pyrimidine | 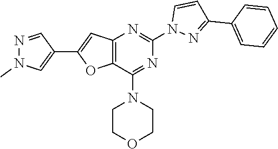 [SMILES: Cn1cc(-c2cc3nc(-n4ccc(-c5ccccc5)n4)nc(N4CCOCC4)c3o2)cn1] | 41 | LC-MS (ESI+): m/z 428 (MH⁺). ¹HNMR (300 MHz, CDCl₃) δ 8.55 (d, J = 2.7 Hz, 1H), 8.01 (d, J = 8.4 Hz, 2H), 7.86 (s, 1H), 7.80 (s, 1H), 7.45-7.32 (m, 3H), 6.80 (s, 1H), 6.77 (d, J = 2.7 Hz, 1H), 4.14-4.09 (m, 4H), 4.00 (s, 3H), 3.94-3.90 (m, 4H). |
| 209 | 4-morpholino-2-(3- phenylpyrazol-1-yl)-6- (1H-pyrazol-4- yl)furo[3,2- d]pyrimidine | 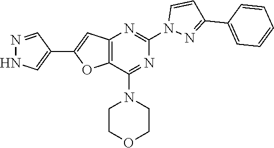 [SMILES: c1ccc(-c2ccn(-c3nc(N4CCOCC4)c4oc(-c5cn[nH]c5)cc4n3)n2)cc1] | 41 | LC-MS (ESI+): m/z 414 (MH⁺). ¹HNMR (300 MHz, CDCl₃) δ 8.56 (d, J = 2.1 Hz, 1H), 8.02-7.99 (m, 4H), 7.46-7.33 (m, 3H), 6.89 (s, 1H), 6.78 (d, J = 2.4 Hz, 1H), 4.15-4.10 (m, 4H), 3.94-3.90 (m, 4H). |
| 210 | 6-(3-methylisothiazol- 5-yl)-4-morpholino-2- [3-(m-tolyl)pyrazol-1- yl]furo[3,2- d]pyrimidine | 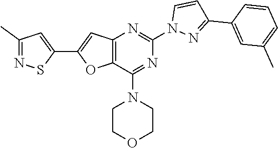 [SMILES: Cc1cccc(-c2ccn(-c3nc(N4CCOCC4)c4oc(-c5cc(C)ns5)cc4n3)n2)c1] | 41 | LC-MS (ESI+): m/z 459 (MH⁺). ¹HNMR (300 MHz, CDCl₃) δ 8.54 (d, J = 2.7 Hz, 1H), 7.90 (s, 1H), 7.76 (d, J = 7.5 Hz, 1H), 7.39 (s, 1H), 7.32 (t, J = 7.5 Hz, 1H), 7.22- 7.18 (m, 2H), 6.78 (d, J = 2.7 Hz, 1H), 4.15-4.11 (m, 4H), 3.95-3.91 (m, 4H), 2.58 (s, 3H), 2.42 (s, 3H). |
| 211 | 6-(3-methylisothiazol- 5-yl)-4-morpholino-2- (3-phenylpyrazol-1- yl)furo[3,2- d]pyrimidine | 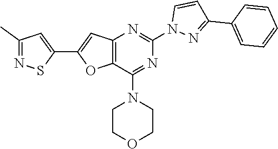 [SMILES: Cc1cc(-c2cc3nc(-n4ccc(-c5ccccc5)n4)nc(N4CCOCC4)c3o2)sn1] | 41 | LC-MS (ESI+): m/z 445 (MH⁺). ¹HNMR (300 MHz, DMSO-d₆) δ 8.72 (d, J = 2.7 Hz, 1H), 7.97 (d, J = 7.2 Hz, 2H), 7.90 (s, 1H), 7.68 (s, 1H), 7.50-7.45 (m, 2H), 7.41-7.36 (m, 1H), 7.05 (d, J = 2.7 Hz, 1H), 4.14-4.09 (m, 4H), 3.85-3.80 (m, 4H), 2.50 (s, 3H). |
| 212 | 6-(2-methyl-1H- imidazol-5-yl)-4- morpholino-2-[3-(m- tolyl)pyrazol-1- yl]furo[3,2- d]pyrimidine | 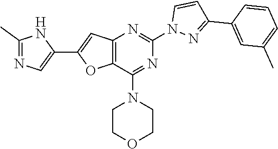 [SMILES: Cc1cccc(-c2ccn(-c3nc(N4CCOCC4)c4oc(-c5cnc(C)[nH]5)cc4n3)n2)c1] | 42 | LC-MS (ESI+): m/z 442 (MH⁺). ¹HNMR (300 MHz, CDCl₃) δ 8.53 (d, J = 2.7 Hz, 1H), 7.82 (s, 1H), 7.70 (d, J = 7.5 Hz, 1H), 7.43 (s, 1H), 7.31-7.26 (m, 1H), 7.14 (d, J = 7.8 Hz, 1H), 7.04 (s, 1H), 6.76 (d, J = 2.7 Hz, 1H), 4.15-4.11 (m, 4H), 3.95-3.91 (m, 4H), 2.51 (s. 3H), 2.41 (s, 3H). |
| 213 | 6-(2-methyl-1H- imidazol-5-yl)-4- morpholino-2-(3- phenylpyrazol-1- yl)furo[3,2- d]pyrimidine | 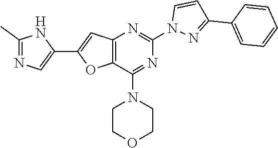 [SMILES: Cc1ncc(-c2cc3nc(-n4ccc(-c5ccccc5)n4)nc(N4CCOCC4)c3o2)[nH]1] | 42 | LC-MS (ESI+): m/z 428 (MH⁺). ¹HNMR (300 MHz, CDCl₃) δ 10.18 (brs, 1H), 8.55 (d, J = 2.7 Hz, 1H), 7.94 (d, J = 7.5 Hz, 2H), 7.39-7.26 (m, 4H), 7.00 (s, 1H), 6.76 (d, J = 2.7 Hz, 1H), 4.17-4.10 (m, 4H), 3.97- 3.88 (m, 4H), 2.53 (s, 3H). |
| 214 | 4-morpholino-6- oxazol-2-yl-2-(3- phenylpyrazol-1- yl)furo[3,2- d]pyrimidine | 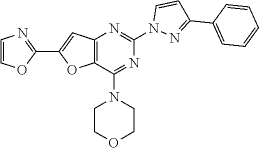 [SMILES: c1ccc(-c2ccn(-c3nc(N4CCOCC4)c4oc(-c5ncco5)cc4n3)n2)cc1] | 41 | LC-MS (ESI+): m/z 415 (MH⁺). ¹HNMR (300 MHz, CDCl₃) δ 8.55 (d, J = 2.7 Hz, 1H), 8.01 (d, J = 7.2 Hz, 2H), 7.83 (s, 1H), 7.61-7.30 (m, 5H), 6.79 (d, J = 2.7 Hz, 1H), 4.19- 4.12 (m, 4H), 3.97-3.89 (m, 4H). |
| 215 | 6-(1-methylpyrazol- 3-yl)-4-morpholino- 2-[4-[3- (trideuteriomethyl) phenyl]pyrazol-1- yl]furo[3,2- d]pyrimidine | 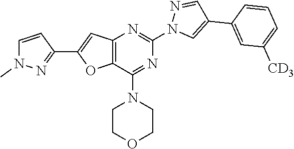 [SMILES: [2H]C([2H])([2H])c1cccc(-c2cnn(-c3nc(N4CCOCC4)c4oc(-c5ccn(C)n5)cc4n3)c2)c1] | 43 and 37 | LC-MS (ESI+): m/z 445 (MH⁺). ¹HNMR (300 MHz, CDCl₃) δ 8.72 (s, 1H), 8.07 (s, 1H), 7.51-7.41 (m, 3H), 7.30-7.26 (m, 1H), 7.11-7.08 (m, 2H), 6.66 (d, J = 2.1 Hz, 1H), 4.22-4.11 (m, 4H), 4.02 (s, 3H), 3.95-3.87 (m, 4H). |
| 216 | 6-(1-methylpyrazol-3- yl)-4-morpholino- 2-[3-[3- (trideuteriomethyl) phenyl]pyrazol-1- yl]furo[3,2- d]pyrimidine | 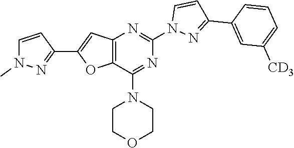 [SMILES: [2H]C([2H])([2H])c1cccc(-c2ccn(-c3nc(N4CCOCC4)c4oc(-c5ccn(C)n5)cc4n3)n2)c1] | 43 and 37 | LC-MS (ESI+): m/z 445 (MH⁺). ¹HNMR (300 MHz, CD₃OD) δ 8.87 (s, 1H), 7.81-7.71 (m, 3H), 7.30-7.26 (m, 1H), 7.19-7.08 (m, 2H), 6.86- 6.83 (m, 2H), 4.18-4.11 (m, 4H), 4.05 (s, 3H), 3.92-3.85 (m, 4H). |
| 217 | 4-morpholino-6-(2- pyridyl)-2-[3-[3- (trideuteriomethyl) phenyl]pyrazol-1- yl]furo[3,2- d]pyrimidine | 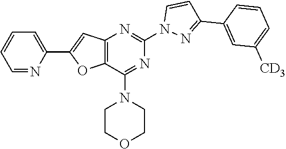 [SMILES: [2H]C([2H])([2H])c1cccc(-c2ccn(-c3nc(N4CCOCC4)c4oc(-c5ccccn5)cc4n3)n2)c1] | 33 and 37 | LC-MS (ESI+): m/z 442 (MH⁺). ¹HNMR (300 MHz, CDCl₃) δ 8.78 (d, J = 2.7 Hz, 1H), 8.56 (s, 1H), 7.83 (s, 1H), 7.79-7.76 (m, 3H), 7.56 (s, 1H), 7.37-7.27 (m, 2H), 7.18-7.16 (m, 1H), 6.78 (d, J = 2.7 Hz, 1H), 4.25-4.14 (m, 4H), 4.02-3.93 (m, 4H). |
| 218 | 4-morpholino-2-[3-(m- tolyl)pyrazol-1-yl]-6- oxazol-2-yl-furo[3,2- d]pyrimidine | 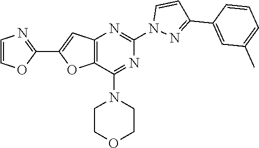 [SMILES: Cc1cccc(-c2ccn(-c3nc(N4CCOCC4)c4oc(-c5ncco5)cc4n3)n2)c1] | 41 | LC-MS (ESI+): m/z 429 (MH⁺). ¹HNMR (300 MHz, DMSO-d₆) δ 8.72 (d, J = 2.7 Hz, 1H), 8.44 (s, 1H), 7.80-7.73 (m, 3H), 7.58 (s, 1H), 7.36 (t, J = 7.5 Hz, 1H), 7.20 (d, J = 7.5 Hz, 1H), 7.02 (d, J = 2.7 Hz, 1H), 4.14-4.08 (m, 4H), 3.91-3.84 (m, 4H), 2.40 (s, 3H). |
| 219 | 4-morpholino-2-[3-(m- tolyl)pyrazol-1-yl]-6- (1H-pyrazol-4- yl)furo[3,2- d]pyrimidine | 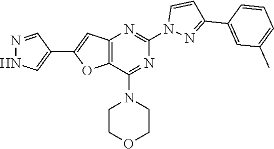 [SMILES: Cc1cccc(-c2ccn(-c3nc(N4CCOCC4)c4oc(-c5cn[nH]c5)cc4n3)n2)c1] | 41 | LC-MS (ESI+): m/z 429 (MH⁺). ¹HNMR (300 MHz, DMSO-d₆) δ 13.39 (s, 1H), 8.69 (d, J = 2.7 Hz, 1H), 8.52 (s, 1H), 8.15 (s, 1H), 7.79- 7.73 (m, 2H), 7.35 (t, J = 7.5 Hz, 1H), 7.20 (d, J = 7.8 Hz, 1H), 7.12 (s, 1H), 7.00 (d, J = 2.7 Hz, 1H), 4.10-4.03 (m, 4H), 3.83-3.78 (m, 4H), 2.40 (s, 3H). |
| 220 | 2-[3-(3- bromophenyl)pyrazol- 1-yl]-4-morpholino-N- tetrahydropyran-4-yl- furo[3,2-d]pyrimidine- 6-carboxamide | 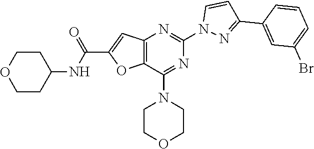 [SMILES: O=C(NC1CCOCC1)c1cc2nc(-n3ccc(-c4cccc(Br)c4)n3)nc(N3CCOCC3)c2o1] | 38 and 37 | LCMS m/z: 552.77, 554.75; ¹HNMR (300 MHz, DMSO-d6) δ 8.76 (s, 1H), 8.69 (d, J = 7.8 Hz, 1H), 8.15 (s, 1H), 7.98 (d, J = 7.5 Hz, 1H), 7.61- 7.57 (m, 2H), 7.45 |
| 221 | 2-[3-(3- methoxyphenyl) pyrazol-1-yl]-4- morpholino-N- tetrahydropyran-4-yl- furo[3,2-d]pyrimidine- 6-carboxamide | 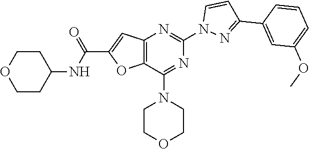 | 38 and 37 | LCMS m/z: 505.05; ¹H NMR (300 MHz, CDCl3) δ 8.70 (s, 1H), 8.07 (s, 1H), 7.47 (s, 1H), 7.35 (t, J = 7.8 Hz, 1H), 7.20 (d, J = 7.8 Hz, 1H), 7.14 (s, 1H), 6.84 (dd, J = 2.1, 8.1 Hz, 1H), 6.20 (d, J = 8.1 Hz, 1H), 4.32-4.20 (m, 1H), 4.18-4.12 (m, 4H), 4.08- 4.02 (m, 2H), 3.95-3.88 (m, 4H), 3.86 (s, 3H), 3.60-3.49 (m, 2H), 2.05-2.02 (m, 2H), 1.67-1.59 (m, 2H). |
| 222 | 4-morpholino-N- tetrahydropyran- 4-yl-2-[4-[3- (trideuteriomethyl) phenyl]pyrazol-1- yl]furo[3,2- d]pyrimidine-6- carboxamide | 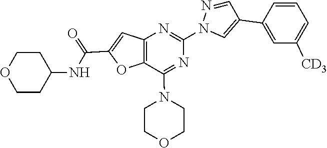 | 38 and 37 | LCMS m/z: 492.15 (M + H)⁺; ¹HNMR (300 MHz, CDCl3) δ 8.52 (d, J = 2.4 Hz, 1H), 7.87 (s, 1H), 7.74 (d, J = 7.5 Hz, 1H), 7.51 (s, 1H), 7.34-7.30 (m, 1H), 7.16 (d, J = 7.5 Hz, 1H), 6.78 (d, J = 2.4 Hz, 1H), 6.29 (d, J = 7.8 Hz, 1H), 4.28-4.18 (m, 1H), 4.16-4.11 (m, 4H), 4.04- 4.00 (m, 2H), 3.95-3.91 (m, 4H), 3.54 (t, J = 11.4 Hz, 2H), 2.04-2.00 (m, 2H), 1.68-1.63 (m, 2H). |
| 223 | 2-[4-(3- methoxyphenyl) pyrazol-1-yl]-4- morpholino-N- tetrahydropyran-4-yl- furo[3,2-d]pyrimidine- 6-carboxamide | 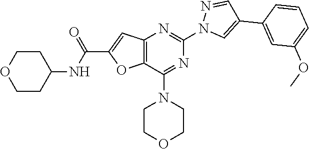 | 38 and 37 | LCMS m/z: 504.88 (M + H)⁺; ¹HNMR (300 MHz, CDCl3) δ 8.53 (d, J = 2.4 Hz, 1H), 7.57-7.51 (m, 3H), 7.33 (t, J = 7.8 Hz, 1H), 6.91 (dd, J = 8.1, 1.8 Hz, 1H), 6.78 (d, J = 2.4 Hz, 1H), 6.33 (d, J = 8.1 Hz, 1H), 4.25-4.23 (m, 1H), 4.17-4.12 (m, 4H), 4.03-4.00 (m, 2H), 3.94-3.88 (m, 7H), 3.54 (t, J = 11.4 Hz, 2H), 2.05-2.00 (m, 2H), 1.67-1.64 (m, 2H). |
| 224 | N,N-dimethyl-2-(1- methyl-3-(4- morpholino-2-(3- (m-tolyl)-1H-pyrazol- 1-yl)furo[3,2- d]pyrimidin-6-yl)-1H- pyrazol-5-yl)ethan- 1-amine | 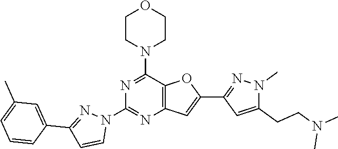 [SMILES: Cc1cccc(-c2ccn(-c3nc(N4CCOCC4)c4oc(-c5cc(CCN(C)C)n(C)n5)cc4n3)n2)c1] | 43 | LCMS m/z: 513.25 (M + H)⁺; ¹HNMR (300 MHz,CDCl3) δ 8.55 (s, J = 2.4 Hz, 1H), 7.91 (s, 1H), 7.74 (d, J = 7.8 Hz, 1H), 7.75-7.28 (m, 1H), 7.15 (d, J = 7.8 Hz, 1H), 7.10 (s, 1H), 6.76 (d, J = 2.4 Hz, 1H), 6.46 (s, 1H), 4.18-4.13 (m, 4H), 3.94-3.89 (m, 7H), 2.91-2.86 (m, 2H), 2.71- 2.66 (m, 2H), 2.41 (s, 3H), 2.39 (s, 6H). |
| 225 | 2-(4-(3-bromophenyl)- 1H-pyrazol-1-yl)-4- morpholino-N- (tetrahydro-2H-pyran- 4-yl)furo[3,2- d]pyrimidine-6- carboxamide | 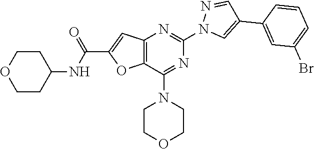 | 38 and 37 | LCMS m/z: 553.14, 555.04 (M + H)⁺; 1H NMR (300 MHz, DMSO-d6) δ 9.15(s, 1H), 8.68 (d, J = 7.8 Hz, 1H), 8.33(s, 1H), 8.06 (s, 1H), 7.83 (d, J = 7.5Hz, 1H), 7.58 (s, 1H), 7.45 (d, J = 8.1Hz, 1H), 7.36 (t, J = 7.8 Hz, 1H), 4.19-4.11 (m, 4H), 4.10-4.00 (m, 1H), 3.97-3.89 (m, 2H), 3.87-3.78(m, 4H), 3.44-3.37 (m, 2H), 1.87-1.78 (m, 2H), 1.74-1.58 (m, 2H). |

Compounds 226-257 in Table 5 are prepared using methods analogous to those described herein.

TABLE 5

| Compound # | Name | Structure |
|---|---|---|
| 226 | 4-morpholino-2-(4-phenyl-1H- pyrazol-1-yl)furo[3,2-d]pyrimidine | 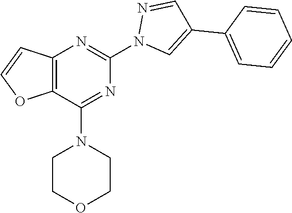 [SMILES: c1ccc(-c2cnn(-c3nc(N4CCOCC4)c4occc4n3)c2)cc1] |
| 227 | morpholino(4-morpholino-2-(3-(m- tolyl)-1H-pyrazol-1-yl)furo[3,2- d]pyrimidin-6-yl)methanone | 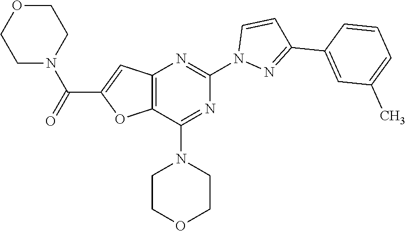 [SMILES: Cc1cccc(-c2ccn(-c3nc(N4CCOCC4)c4oc(C(=O)N5CCOCC5)cc4n3)n2)c1] |
| 228 | (4-methylpiperazin-1-yl)(4- morpholino-2-(3-(m-tolyl)-1H- pyrazol-1-yl)furo[3,2-d] pyrimidin-6-yl)methanone | 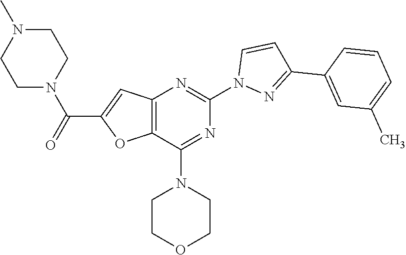 [SMILES: Cc1cccc(-c2ccn(-c3nc(N4CCOCC4)c4oc(C(=O)N5CCN(C)CC5)cc4n3)n2)c1] |
| 229 | N,N-dimethyl-4-morpholino-2-(3- (m-tolyl)-1H-pyrazol-1-yl)furo[3,2- d]pyrimidine-6-carboxamide | 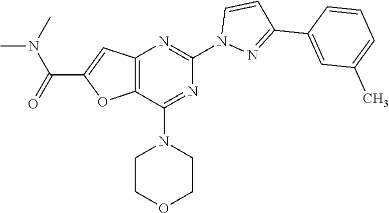 [SMILES: Cc1cccc(-c2ccn(-c3nc(N4CCOCC4)c4oc(C(=O)N(C)C)cc4n3)n2)c1] |
| 230 | N-methoxy-N-methyl-4-morpholino- 2-(3-(m-tolyl)-1H-pyrazol-1- yl)furo[3,2-d]pyrimidine-6- carboxamide | 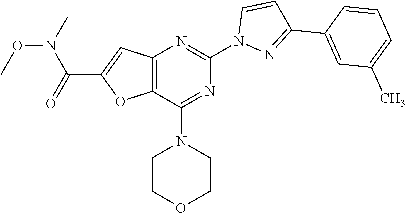 [SMILES: CON(C)C(=O)c1cc2nc(-n3ccc(-c4cccc(C)c4)n3)nc(N3CCOCC3)c2o1] |
| 231 | (4-morpholino-2-(3-(m-tolyl)-1H- pyrazol-1-yl)furo[3,2-d]pyrimidin-6- yl)(piperidin-1-yl)methanone | 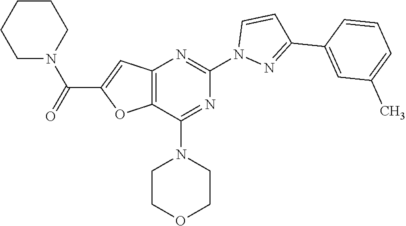 [SMILES: Cc1cccc(-c2ccn(-c3nc(N4CCOCC4)c4oc(C(=O)N5CCCCC5)cc4n3)n2)c1] |
| 232 | azetidin-1-yl(4-morpholino-2-(3-(m- tolyl)-1H-pyrazol-1-yl)furo[3,2- d]pyrimidin-6-yl)methanone | 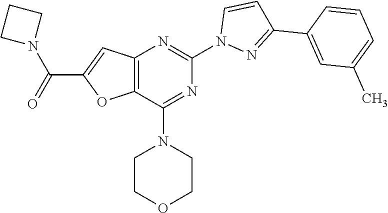 [SMILES: Cc1cccc(-c2ccn(-c3nc(N4CCOCC4)c4oc(C(=O)N5CCC5)cc4n3)n2)c1] |
| 233 | (4-morpholino-2-(3-(m-tolyl)-1H- pyrazol-1-yl)furo[3,2-d]pyrimidin-6- yl)(pyrrolidin-1-yl)methanone | 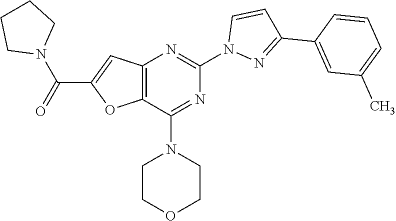 [SMILES: Cc1cccc(-c2ccn(-c3nc(N4CCOCC4)c4oc(C(=O)N5CCCC5)cc4n3)n2)c1] |
| 234 | N-ethyl-N-methyl-4-morpholino-2- (3-(m-tolyl)-1H-pyrazol-1- yl)furo[3,2-d]pyrimidine-6- carboxamide | 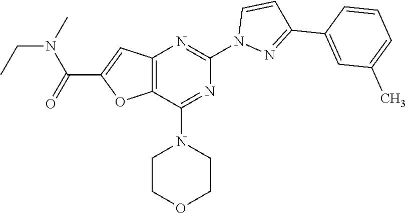 [SMILES: CCN(C)C(=O)c1cc2nc(-n3ccc(-c4cccc(C)c4)n3)nc(N3CCOCC3)c2o1] |
| 235 | N-cyclopropyl-N-methyl-4- morpholino-2-(3-(m-tolyl)-1H- pyrazol-1-yl)furo[3,2-d]pyrimidine- 6-carboxamide | 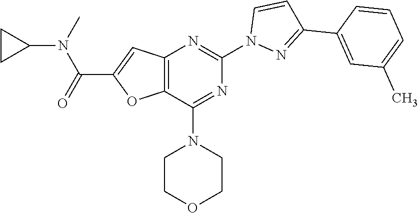 [SMILES: Cc1cccc(-c2ccn(-c3nc(N4CCOCC4)c4oc(C(=O)N(C)C5CC5)cc4n3)n2)c1] |
| 236 | N-(cyclopropylmethyl)-N-methyl-4- morpholino-2-(3-(m-tolyl)-1H- pyrazol-1-yl)furo[3,2-d]pyrimidine- 6-carboxamide | 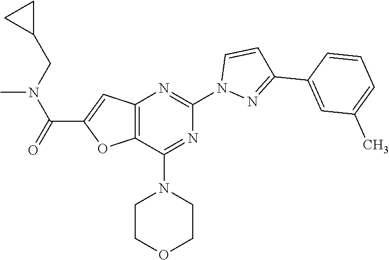 [SMILES: Cc1cccc(-c2ccn(-c3nc(N4CCOCC4)c4oc(C(=O)N(C)CC5CC5)cc4n3)n2)c1] |
| 237 | 4-morpholino-6-(pyrimidin-4-yl)-2- (4-(m-tolyl)-1H-pyrazol-1- yl)furo[3,2-d]pyrimidine | 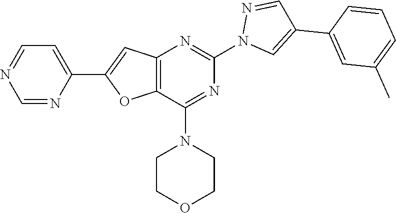 [SMILES: Cc1cccc(-c2cnn(-c3nc(N4CCOCC4)c4oc(-c5ccncn5)cc4n3)c2)c1] |
| 238 | 6-(3-methyl-1H-pyrazol-5-yl)-4- morpholino-2-(4-(m-tolyl)-1H- pyrazol-1-yl)furo[3,2-d]pyrimidine | 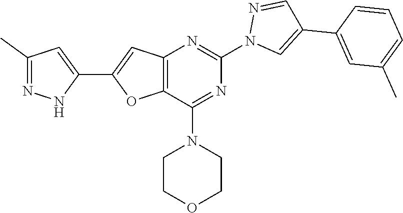 [SMILES: Cc1cccc(-c2cnn(-c3nc(N4CCOCC4)c4oc(-c5cc(C)n[nH]5)cc4n3)c2)c1] |
| 239 | N,N-dimethyl-2-(3-methyl-5-(4- morpholino-2-(4-(m-tolyl)-1H- pyrazol-1-yl)furo[3,2-d]pyrimidin-6- yl)-1H-pyrazol-1-yl)ethan-1-amine | 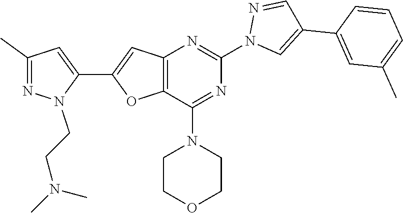 [SMILES: Cc1cccc(-c2cnn(-c3nc(N4CCOCC4)c4oc(-c5cc(C)nn5CCN(C)C)cc4n3)c2)c1] |
| 240 | N,N-dimethyl-2-(5-methyl-3-(4- morpholino-2-(4-(m-tolyl)-1H- pyrazol-1-yl)furo[3,2-d]pyrimidin-6- yl)-1H-pyrazol-1-yl)ethan-1-amine | 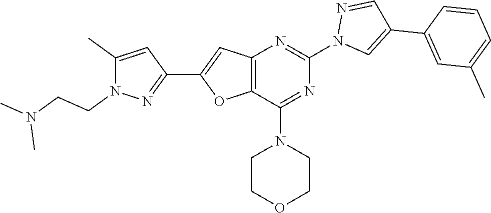 [SMILES: Cc1cccc(-c2cnn(-c3nc(N4CCOCC4)c4oc(-c5cc(C)n(CCN(C)C)n5)cc4n3)c2)c1] |
| 241 | N,N-dimethyl-1-(6-(4-morpholino- 2-(4-(m-tolyl)-1H-pyrazol-1- yl)furo[3,2-d]pyrimidin-6- yl)pyridin-3-yl)methanamine | 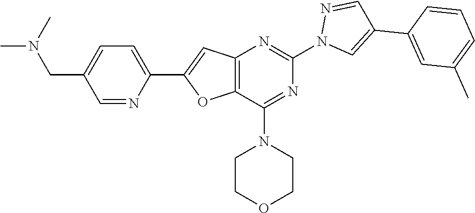 [SMILES: Cc1cccc(-c2cnn(-c3nc(N4CCOCC4)c4oc(-c5ccc(CN(C)C)cn5)cc4n3)c2)c1] |
| 242 | N-(2-(dimethylamino)ethyl)-6-(4- morpholino-2-(4-(m-tolyl)-1H- pyrazol-1-yl)furo[3,2-d]pyrimidin-6- yl)nicotinamide | 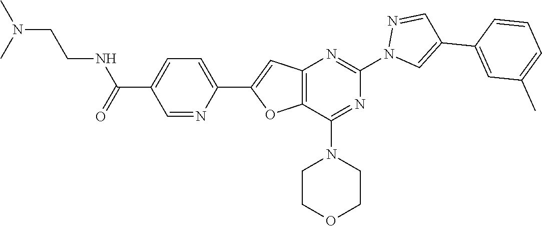 [SMILES: Cc1cccc(-c2cnn(-c3nc(N4CCOCC4)c4oc(-c5ccc(C(=O)NCCN(C)C)cn5)cc4n3)c2)c1] |
| 243 | N1,N1-dimethyl-N2-(6-(4- morpholino-2-(4-(m-tolyl)-1H- pyrazol-1-yl)furo[3,2-d]pyrimidin-6- yl)pyridin-2-yl)ethane-1,2-diamine | 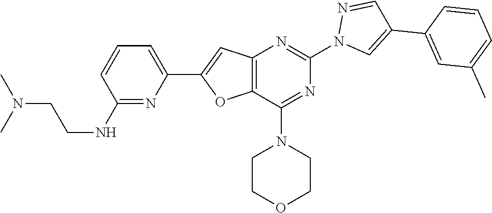 [SMILES: Cc1cccc(-c2cnn(-c3nc(N4CCOCC4)c4oc(-c5cccc(NCCN(C)C)n5)cc4n3)c2)c1] |
| 244 | N,N-dimethyl-2-(5-(4-morpholino-2- (4-(m-tolyl)-1H-pyrazol-1- yl)furo[3,2-d]pyrimidin-6-yl)-1H- pyrazol-3-yl)ethan-1-amine | 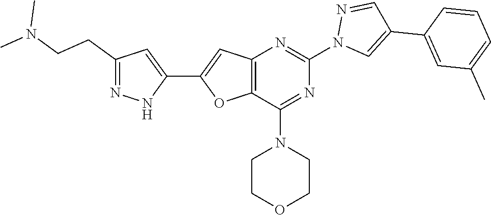 [SMILES: Cc1cccc(-c2cnn(-c3nc(N4CCOCC4)c4oc(-c5cc(CCN(C)C)n[nH]5)cc4n3)c2)c1] |
| 245 | N-ethyl-4-(3-oxomorpholino)-2-(4- (m-tolyl)-1H-pyrazol-1-yl)furo[3,2- d]pyrimidine-6-carboxamide | 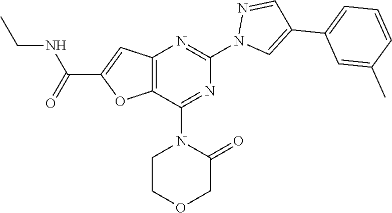 [SMILES: CCNC(=O)c1cc2nc(-n3cc(-c4cccc(C)c4)cn3)nc(N3CCOCC3=O)c2o1] |
| 246 | N-ethyl-4-(3-oxopiperazin-1-yl)-2- (4-(m-tolyl)-1H-pyrazol-1- yl)furo[3,2-d]pyrimidine-6- carboxamide | 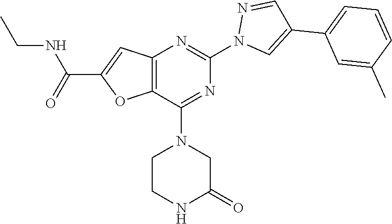 [SMILES: CCNC(=O)c1cc2nc(-n3cc(-c4cccc(C)c4)cn3)nc(N3CCNC(=O)C3)c2o1] |
| 247 | N-(1H-pyrazol-4-yl)-6-(pyridin-3- yl)-2-(4-(m-tolyl)-1H-pyrazol-1- yl)furo[3,2-d]pyrimidin-4-amine | 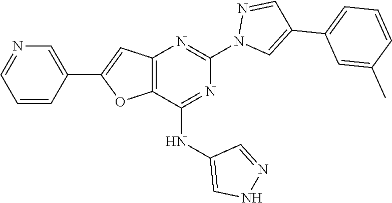 [SMILES: Cc1cccc(-c2cnn(-c3nc(Nc4cn[nH]c4)c4oc(-c5cccnc5)cc4n3)c2)c1] |
| 248 | N-(1H-pyrazol-3-yl)-6-(pyridin-3- yl)-2-(4-(m-tolyl)-1H-pyrazol-1- yl)furo[3,2-d]pyrimidin-4-amine | 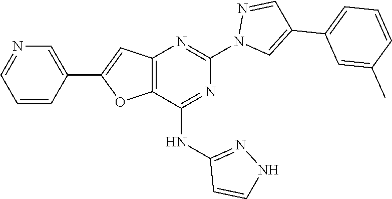 [SMILES: Cc1cccc(-c2cnn(-c3nc(Nc4cc[nH]n4)c4oc(-c5cccnc5)cc4n3)c2)c1] |
| 249 | l-(6-(pyridin-3-yl)-2-(4-(m-tolyl)- 1H-pyrazol-1-yl)furo[3,2- d]pyrimidin-4-yl)-1H-pyrazol-4- amine | 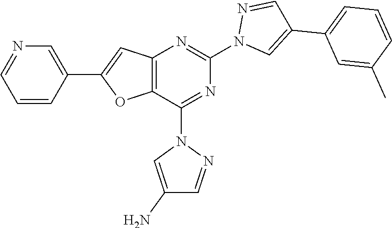 [SMILES: Cc1cccc(-c2cnn(-c3nc(-n4cc(N)cn4)c4oc(-c5cccnc5)cc4n3)c2)c1] |
| 250 | 1-(6-(pyridin-3-yl)-2-(4-(m-tolyl)- 1H-pyrazol-1-yl)furo[3,2- d]pyrimidin-4-yl)-1H-pyrazol-3- amine | 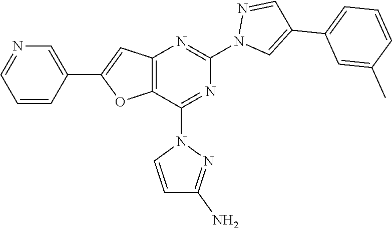 [SMILES: Cc1cccc(-c2cnn(-c3nc(-n4ccc(N)n4)c4oc(-c5cccnc5)cc4n3)c2)c1] |
| 251 | N-(1H-imidazol-4-yl)-6-(pyridin-3- yl)-2-(4-(m-tolyl)-1H-pyrazol-1- yl)furo[3,2-d]pyrimidin-4-amine | 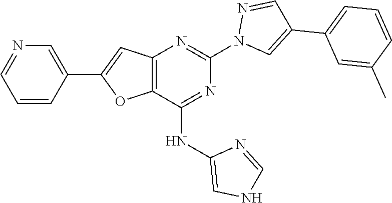 [SMILES: Cc1cccc(-c2cnn(-c3nc(Nc4c[nH]cn4)c4oc(-c5cccnc5)cc4n3)c2)c1] |
| 252 | 1-(6-(pyridin-3-yl)-2-(4-(m-tolyl)- 1H-pyrazol-1-yl)furo[3,2- d]pyrimidin-4-yl)-1H-imidazol-4- amine | 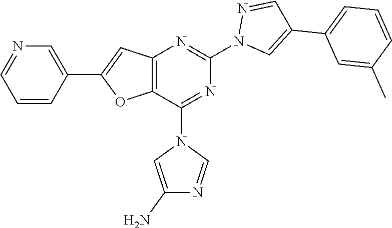 [SMILES: Cc1cccc(-c2cnn(-c3nc(-n4cnc(N)c4)c4oc(-c5cccnc5)cc4n3)c2)c1] |
| 253 | N,N-dimethyl-2-(1-methyl-3-(4- morpholino-2-(4-phenyl-1H- pyrazol-1-yl)furo[3,2-d]pyrimidin-6- yl)-1H-pyrazol-5-yl)ethan-1-amine | 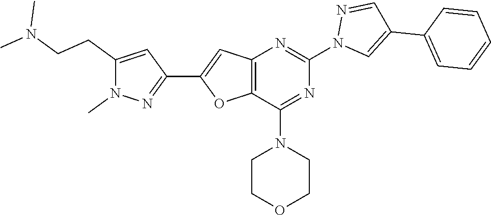 [SMILES: CN(C)CCc1cc(-c2cc3nc(-n4cc(-c5ccccc5)cn4)nc(N4CCOCC4)c3o2)nn1C] |
| 254 | N,N-dimethyl-2-(1-methyl-3-(4- morpholino-2-(3-phenyl-1H- pyrazol-1-yl)furo[3,2-d]pyrimidin-6- yl)-1H-pyrazol-5-yl)ethan-1-amine | 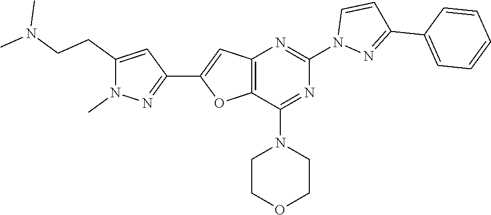 [SMILES: CN(C)CCc1cc(-c2cc3nc(-n4ccc(-c5ccccc5)n4)nc(N4CCOCC4)c3o2)nn1C] |
| 255 | 6-(1H-imidazol-2-yl)-4-morpholino- 2-(3-(m-tolyl)-1H-pyrazol-1- yl)furo[3,2-d]pyrimidine | 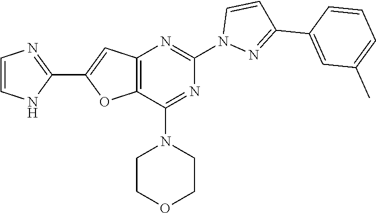 [SMILES: Cc1cccc(-c2ccn(-c3nc(N4CCOCC4)c4oc(-c5ncc[nH]5)cc4n3)n2)c1] |
| 256 | 6-(1H-imidazol-2-yl)-4-morpholino- 2-(3-phenyl-1H-pyrazol-1- yl)furo[3,2-d]pyrimidine | 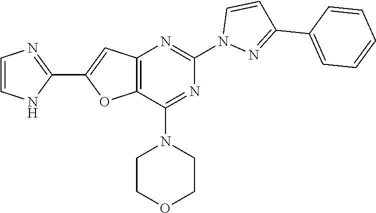 [SMILES: c1ccc(-c2ccn(-c3nc(N4CCOCC4)c4oc(-c5ncc[nH]5)cc4n3)n2)cc1] |
| 257 | N,N-dimethyl-2-(1-methyl-3-(4- morpholino-2-(4-(m-tolyl)-1H- pyrazol-1-yl)furo[3,2-d]pyrimidin-6- yl)-1H-pyrazol-5-yl)ethan-1-amine | 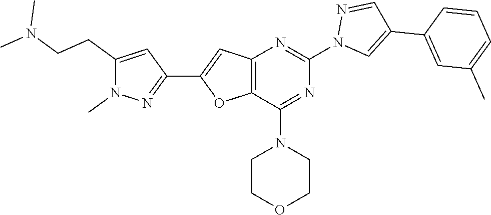 [SMILES: Cc1cccc(-c2cnn(-c3nc(N4CCOCC4)c4oc(-c5cc(CCN(C)C)n(C)n5)cc4n3)c2)c1] |

### Biological Example 1: Inhibition of PIKfyve

Full length human recombinant PIKFYVE expressed in baculovirus expression system as N-terminal GST-fusion protein (265 kDa) was obtained from Carna Biosciences (Kobe, Japan). The kinase substrate was prepared by mixing and sonicating fluorescently-labeled phosphatidylinositol 3-phosphate (PI3P) with phospho-L-serine (PS) at a 1:10 ratio in 50 mM HEPES buffer pH 7.5.

The kinase reactions were assembled in 384-well plates (Greiner) in a total volume of 20 mL as follows. Kinase protein was pre-diluted in an assay buffer comprising 25 mM HEPES, pH 7.5, 1 mM DTT, 2.5 mM MgCl₂, and 2.5 mM MnCl₂, and 0.005% Triton X-100, and dispensed into a 384-well plate (10 μL per well). Test compounds were serially pre-diluted in DMSO and added to the protein samples by acoustic dispensing (Labcyte Echo). The concentration of DMSO was equalized to 1% in all samples. All test compounds were tested at 12 concentrations. Apilimod was used as a reference compound and was tested in identical manner in each assay plate. Control samples (0%-inhibition, in the absence of inhibitor, DMSO only) and 100%-inhibition (in the absence of enzyme) were assembled in replicates of four and were used to calculate %-inhibition in the presence of compounds. The reactions were initiated by addition of 10 μL of 2×PI3P/PS substrate supplemented with ATP. The final concentration of enzyme was 2 nM, the final concentration of ATP was 10 mM, and the final concentration of PI3P/PS substrate was 1 μM (PI3P). The kinase reactions were allowed to proceed for 3 h at room temperature. Following incubation, the reactions were quenched by addition of 50 mL of termination buffer (100 mM HEPES, pH 7.5, 0.01% Triton X-100, 20 mM EDTA). Terminated plates were analyzed on a microfluidic electrophoresis instrument (Caliper LabChip® 3000, Caliper Life Sciences/Perkin Elmer). The change in the relative fluorescence intensity of the PI(3)P substrate and PI(3,5)P product peaks was measured. The activity in each test sample was determined as the product to sum ratio (PSR): P/(S+P), where P is the peak height of the product, and S is the peak height of the substrate. Percent inhibition (Pᵢₙₕ) was determined using the following equation:

Pᵢₙₕ=(PSR_{0% inh}−PSR_{compound})/(PSR_{0% inh}−PSR_{100% inh})*100

in which PSR_{compound} is the product/sum ratio in the presence of compound, PSR0_{% inh} is the product/sum ratio in the absence of compound, and the PSR_{100% inh} is the product/sum ratio in the absence of the enzyme. To determine the IC₅₀ of test compounds (50%-inhibition) the %-inh cdata (Pᵢₙₕ versus compound concentration) were fitted by a four-parameter sigmoid dose-response model using XLfit software (IDBS).

The IC₅₀ values for certain compounds of the disclosure are provided in Table 5 below.

TABLE 6

| | PIKfyve |
|---|---|
| Compound # | IC₅₀ (nM) |
| 1 | 1.4 |
| 2 | 64.8 |
| 3 | 0.8 |
| 4 | 30.9 |
| 5 | 279 |
| 6 | 44.6 |
| 7 | 451 |
| 8 | 59.5 |
| 9 | >10000 |
| 10 | 6220 |
| 11 | 54.4 |
| 12 | 116 |
| 13 | 545 |
| 14 | 847 |
| 15 | 71 |
| 16 | 1000 |
| 17 | 303 |
| 18 | 144 |
| 19 | 64 |
| 20 | 9 |
| 21 | 27 |
| 22 | 27 |
| 23 | 195 |
| 24 | 60 |
| 25 | 956 |
| 26 | 0.1 |
| 27 | 1 |
| 28 | 677 |
| 29 | 1 |
| 30 | 0.5 |
| 31 | 369 |
| 32 | 116 |
| 33 | 59 |
| 34 | 10 |
| 35 | 205 |
| 36 | 37 |
| 37 | 10000 |
| 38 | 131 |
| 39 | 2,560 |
| 40 | 2,570 |
| 41 | 34 |
| 42 | 9 |
| 43 | 8 |
| 44 | 2 |
| 45 | 48 |
| 46 | 107 |
| 47 | 52 |
| 48 | 32 |
| 49 | 21 |
| 50 | 835 |
| 51 | 88 |
| 52 | 47 |
| 53 | 60 |
| 54 | 66 |
| 55 | 51 |
| 56 | 2 |
| 57 | 37 |
| 58 | 95 |
| 59 | 4.3 |
| 60 | 70.2 |
| 61 | 2.5 |
| 62 | 73 |
| 63 | 0.7 |
| 64 | 0.9 |
| 65 | 235 |
| 66 | 612 |
| 67 | 928 |
| 68 | 185 |
| 69 | 338 |
| 70 | 142 |
| 71 | 79.8 |
| 72 | 552 |
| 73 | 141 |
| 74 | 224 |
| 75 | 1.4 |
| 76 | 1.1 |
| 77 | 175 |
| 78 | 7,850 |
| 79 | 10000 |
| 80 | 5500 |
| 81 | 10000 |
| 82 | 1390 |
| 83 | 4090 |
| 84 | 20000 |
| 85 | 69 |
| 86 | 61 |
| 87 | 20000 |
| 88 | 7400 |
| 89 | 19 |
| 90 | 7.7 |
| 91 | 3.9 |
| 92 | 91.5 |
| 93 | 47.3 |
| 94 | 86.7 |
| 95 | 11.8 |
| 96 | 15 |
| 97 | 17 |
| 98 | 78.7 |
| 99 | 78.3 |
| 100 | 1.2 |
| 101 | 2.9 |
| 102 | 12.1 |
| 103 | 11.2 |
| 104 | 41.2 |
| 105 | 32.3 |
| 106 | 10.2 |
| 107 | 1.9 |
| 108 | 23.3 |
| 109 | 11.7 |
| 110 | 2.9 |
| ill | 3.1 |
| 112 | >10,000 |
| 113 | 0.2 |
| 114 | 9.6 |
| 115 | 3.1 |
| 116 | 49.3 |
| 117 | 37.3 |
| 118 | 311 |
| 119 | 8.2 |
| 120 | 16.2 |
| 121 | 1.2 |
| 122 | 12.7 |
| 123 | 3.3 |
| 124 | 4.7 |
| 125 | 0.9 |
| 126 | 2.9 |
| 127 | 34 |
| 128 | 66.2 |
| 129 | 11.8 |
| 130 | 52.6 |
| 131 | 18 |
| 132 | 39.9 |
| 133 | >20,000 |
| 134 | >20,000 |
| 135 | 147 |
| 136 | 94 |
| 137 | 95 |
| 138 | 33 |
| 139 | 22 |
| 140 | 43 |
| 141 | 252 |
| 142 | 145 |
| 143 | 25 |
| 144 | 0.3 |
| 145 | 18.6 |
| 146 | 0.3 |
| 147 | 1,030 |
| 148 | 3,860 |
| 149 | >20,000 |
| 150 | 103 |
| 151 | 446 |
| 152 | 306 |
| 153 | 24 |
| 154 | 77.3 |
| 155 | 70.6 |
| 156 | 152 |
| 157 | 88.8 |
| 158 | 160 |
| 159 | 943 |
| 160 | 61.7 |
| 161 | 40.4 |
| 162 | 17.8 |
| 163 | 23.5 |
| 164 | 162 |
| 165 | 68.4 |
| 166 | 220 |
| 167 | 122 |
| 168 | 62.1 |
| 169 | 67.3 |
| 170 | 45.5 |
| 171 | 39.2 |
| 172 | 37.8 |
| 173 | 0.6 |
| 174 | 531 |
| 175 | 4,010 |
| 176 | 47.3 |
| 177 | 41.3 |
| 178 | 0.9 |
| 179 | 10,000 |
| 180 | 55.6 |
| 181 | 59.4 |
| 182 | 84.2 |
| 183 | 11.8 |
| 184 | 1,430 |
| 185 | 10.1 |
| 186 | 6 |
| 187 | 2,390 |
| 188 | 2,830 |
| 189 | 0.8 |
| 190 | 12.4 |
| 191 | 51.9 |
| 192 | 21.7 |
| 193 | 24.6 |
| 194 | 269 |
| 195 | 907 |
| 196 | 0.9 |
| 197 | 0.7 |
| 198 | 0.6 |
| 199 | 36.8 |
| 200 | 3,080 |
| 201 | 0.5 |
| 202 | 1.4 |
| 203 | 1.9 |
| 204 | 2.1 |
| 205 | 1.3 |
| 206 | 7 |
| 207 | 6.6 |
| 208 | 8.2 |
| 209 | 9.8 |
| 210 | 3.8 |
| 211 | 20.8 |
| 212 | 2.3 |
| 213 | 10.6 |
| 214 | 44.1 |
| 215 | 0.2 |
| 216 | 0.8 |
| 217 | 2.2 |
| 218 | 9.1 |
| 219 | 0.4 |
| 220 | 16.3 |
| 221 | 3.4 |

### Biological Example 2: Inhibition of PI3K Isoforms

The enzyme preparations shown in Table 6 were used.

TABLE 7

| | | Regulatory | Expression |
|---|---|---|---|
| Assay name | Catalytic subunit | subunit | host |
| PI3Kα | Full-length N-terminal | Full length, | SF9 cells |
| (p110α/p85α) | FLAG-tagged | human | (baculovirus) |
| | human p110α | (no tag) p85α | |
| PI3Kβ | Full-length N-terminal | Full length, | SF9 cells |
| (p110β/p85α) | GST-tagged | human | (baculovirus) |
| | human p110β | (no tag) p85α | |
| PI3Kδ | Full-length N-terminal | Full length, | SF9 cells |
| (p110δ/p85α) | GST-tagged | human | (baculovirus |
| | human p110δ | (no tag) p85α | |
| PI3Kγ (p120γ) | Full length N-terminal | None | SF9 cells |
| | His-tagged | | (baculovirus) |
| | human p120γ | | |

The kinase substrate was prepared by mixing and sonicating fluorescently-labeled phosphatidylinositol 4,5-phosphate (PIP2) with phospho-L-serine (PS) at 1:20 ratio in 50 mM HEPES buffer pH7.5.

The kinase reactions were assembled in 384-well plates (Greiner) in a total volume of 20 mL as follows. The kinase proteins were pre-diluted in an assay buffer comprising 50 mM HEPES, pH 7.5, 0.012% CHAPS, 1 mM DTT, 10 mM Na₃VaO₄, 10 mM 3-GP, 3 mM MgCl₂, and 40 mM NaCl₂, and dispensed into a 384-well plate (10 μL per well). Test compounds were serially pre-diluted in DMSO and added to the protein samples by acoustic dispensing (Labcyte Echo). The concentration of DMSO was equalized to 1% in all samples. All test compounds were tested at 12 concentrations. The control samples (0%-inhibition in the absence of inhibitor, DMSO only) and 100%-inhibition (in the absence of enzyme) were assembled in replicates of four and were used to calculate %-inhibition in the presence of test compounds. The reactions were initiated by addition of 10 μL of the PIP2/PS substrate supplemented with ATP. The final concentration of enzymes was 0.5 nM (PI3Kα), 1 nM (PI3Kβ), 10 nM (PI3Kγ), and 0.25 nM (PI3Kδ). The final concentration of ATP was 90 μM (PI3Kα), 60 μM (PI3Kβ), 100 μM (PI3Kγ), and 90 μM (PI3Kδ). The final concentration of PIP2/PS substrate was 1 μM (PIP2). The kinase reactions were allowed to proceed for 3 h at room temperature. Following incubation, the reactions were quenched by addition of 50 μL of termination buffer (100 mM HEPES, pH 7.5, 0.01% Triton X-100, 20 mM EDTA). Terminated plates were analyzed on a microfluidic electrophoresis instrument (Caliper LabChip® 3000, Caliper Life Sciences/Perkin Elmer). The change in the relative fluorescence intensity of the PI(4,5)P substrate and PI(3,4,5)P product peaks was measured. The activity in each test sample was determined as the product to sum ratio (PSR): P/(S+P), where P is the peak height of the product, and S is the peak height of the substrate. Percent inhibition (Pᵢₙₕ) was determined using the following equation:

Pᵢₙₕ=(PSR_{0% inh}−PSR_{compound})/(PSR_{0% inh}−PSR_{100% inh})*100

in which PSR_{compound} is the product/sum ratio in the presence of compound, PSR_{0% inh} is the product/sum ratio in the absence of compound, and the PSR_{100% inh} is the product/sum ratio in the absence of the enzyme. To determine the IC₅₀ of test compounds (50%-inhibition), the %-inh cdata (Pᵢₙₕ versus compound concentration) were fitted by a four-parameter sigmoid dose-response model using XLfit software (IDBS).

The IC₅₀ values for certain compounds of the disclosure are provided in Table 7 below.

TABLE 8

| | PI3Ka IC₅₀ | PI3Kb IC₅₀ | PI3Kd IC₅₀ | PI3Kg IC₅₀ |
|---|---|---|---|---|
| Compound # | (nM) | (nM) | (nM) | (nM) |
| 1 | 6780 | >10000 | 2710 | >10000 |
| 70 | 4140 | 20000 | 164 | 20000 |
| 71 | 20000 | 20000 | 313 | 20000 |
| 72 | 20000 | 20000 | 20000 | 20000 |
| 80 | 5520 | 20000 | 454 | 20000 |
| 83 | 3720 | 20000 | 20000 | 20000 |
| 84 | 4530 | 20000 | 218 | 20000 |
| 87 | 3480 | 20000 | 58.6 | 20000 |

### Biological Example 3: Cell Data

HEK/TDP Survival assay: Immortalized human embryonic kidney 293T (HEK 293T) were transfected with plasmids containing TDP-43 Q331K mutation, resulting in an increase in cell death that is biologically relevant to ALS patients. Cell death is measured as reductions in the amount of ATP, an indicator of metabolically active cells, that is quantified by a luminescence Cell-Titer-Glo® (CTG) reagent. Compounds are evaluated in this model for changes in CTG compared to a no treatment group. Increased signal indicates improved survival (rescue) and decreased signal indicates decreased survival.

Cell rescue was measured in a 96-well format with eight different concentrations of the test compound over 48 hours (hrs) with 6 replicates. The Promega Cell-Titer-Glo® Luminescent Cell Viability Assay was used to quantify ATP, an indicator of metabolically active cells (see protocol: https://www.promega.com/-/media/files/resources/protocols/technical-manuals/101/celltiterglo-2-0-assay-protocol.pdf?la=en). The luminescence signal was detected using the PerkinElmer EnVision or Molecular Devices SpectraMax.

The effect of a compound at a given dose on cell viability was determined using a three step procedure. First, Hedge's g for the Cell Titer-Glo luminescence values using six untreated wells on every plate as a control was calculated. Second, as multiple experimental trials of each compound-dose pair were performed, these results were meta-analyzed to produce a single estimate of the effect size. Finally, values from all compound-dose pairs were corrected for multiple hypothesis testing using the Empirical Bayes framework of Stephens, M. (False discovery rates: a new deal, Biostatistics, 18 [2], 2017, 275-294) yielding credible intervals for the measured effect and associated s values.

Briefly, this method computes a local false sign rate for each experiment. Analogous to the local false discovery rate of Efron, B. (Size, power and false discovery rates, Ann. Statist. 35 [4], 2007, 1351-1377) this value measures the confidence in the sign of each effect (rather than confidence in each effect being non-zero). The s values reported in the previous figures are the expected fraction of errors if estimating the sign of all effects with greater absolute local false sign rate, defined in analogy to the q value of Storey, JD (The positive false discovery rate: a Bayesian interpretation and the q-value, Ann. Statist. 31 [6] 2003, 2013-2035).

Drugs that yielded signed log s values greater than 3 were considered hits. This threshold was determined by a separate calibration experiment in which Cell Titer-Glo® was measured in blank plates consisting of untreated cells to assess the noise inherent in the assay. Data are presented as the maximal effect of rescue obtained from the dose-response curve.

iPSC MN Survival assay: Fibroblasts from ALS patients with known SOD1 A4V mutation were reprogrammed into inducible pluripotent stem cells (iPSC) and then differentiated to motor neurons. In culture, ALS patient derived motor neurons show increased death rate compared to motor neurons derived from healthy individuals in a stressed condition (nutrient deprived media, Hank's buffered salt solution—HBSS). The SOD1 survival deficit is relevant to a subset of ALS patient biology and serves as a suitable cell-based model for gauging compound induced survival rescue.

Cell rescue was measured following more than two different concentrations of each compound for six days with greater than four replicates in a 96-well format to ensure studies with power>0.8. Cells were transduced with a GFP reporter and imaged once a day to track survival. A broad-spectrum caspase inhibitor served as the positive control.

Microscopy image-based readout: Cells were transduced with a GFP reporter and imaged once a day with a blue laser to track survival. Imagers used include the Biotek Cytation 5 and Thermo Fisher EVOS Auto FL 2. All Images underwent uniform processing consisting of rolling hat background subtraction and contrast adjustment.

Cells were identified by their shape and each cell was tracked across images and time points for each well. Survival was visually assessed from the Kaplan-Meier curves. Survival of the cells was modeled and tested using a mixed effects Cox regression where each well was modeled as the random effect, and the group (control/treatment) as the fixed effect. Hazard ratios between treatment and control were estimated within the Cox regression where a value of 1.0 denotes no change, values>1.0 indicate decreased survival in response to treatment, and values<1.0 indicate increased survival in response to treatment. Data are presented as the maximal reduction in hazard ratio scores measured at various concentrations.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

TABLE 9

| | | C9 iPSC MN |
|---|---|---|
| | HEK/TDP | Significant survival |
| Compound # | EC₅₀ [nM] (n) | rescue EC₅₀ [uM] |
| 1 | 14 (1) | 1.0 |
| 3 | 16.0 (2) | 3.0 |
| 43 | 47.5 (2) | No data |
| 56 | 15.0 (2) | 1.0 |
| 89 | 31.5 (2) | No data |
| 90 | 20.5 (2) | No data |
| 91 | 24.5 (2) | 1.0 |
| 93 | 60 (2) | No data |
| 95 | 94 (2) | No data |
| 96 | 17.5 (2) | No data |
| 97 | 16.5 (2) | No data |
| 100 | 18 (2) | 0.3 |
| 101 | No data | 1.0 |
| 102 | 43 (2) | 1.0 |
| 103 | No data | 1.0 |
| 104 | 98 (2) | No data |
| 105 | 100 (2) | No data |
| 106 | 53.5 (2) | 1 |
| 107 | 59.5 (2) | 1 |
| 108 | 53 (2) | No data |
| 109 | 46.5 (2) | No data |
| 110 | 33 (2) | No data |
| 111 | 35.5 (2) | No data |
| 112 | 153 (2) | No data |
| 113 | 16 (2) | No data |
| 114 | 61 (2) | No data |
| 115 | 35.7 (3) | No data |
| 117 | 74.3 (3) | No data |
| 119 | 96.5 (2) | No data |
| 120 | 121 (1) | No data |
| 121 | 21.5 (2) | No data |
| 123 | 28.5 (2) | No data |
| 124 | 48 (2) | No data |
| 125 | 26 (2) | No data |
| 126 | 30.5 (2) | No data |
| 127 | 48 | No data |
| 129 | 9 | No data |
| 131 | 17 | No data |
| 132 | 15 | No data |
| 143 | 48 | 1.0 |
| 144 | 13 | No data |
| 145 | 27 | No data |
| 146 | 11 | No data |

What is claimed is:

1. A compound of Formula (I): 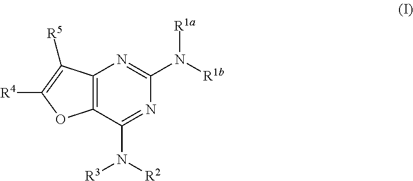
wherein:
R^{1a} and R^{1b} taken together with the nitrogen to which they are attached form: 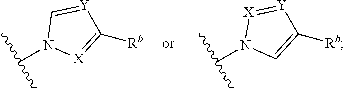
wherein X and Y are independently N or CR^{a};
wherein R^{a} is H or C₁₋₄alkyl; and
R^{b} is phenyl, monocyclic cycloalkyl, monocyclic heterocyclyl, monocyclic heterocycloalkyl, or monocyclic heteroaryl, each optionally substituted with one, two, or three R^{d} substituents;
or R^{1a} is H or C₁₋₄alkyl; and R^{1b} is a heteroaryl optionally substituted with R^{c};
wherein R^{c} is C₁₋₄alkyl, phenyl, —C₁₋₄alkyl-phenyl, monocyclic cycloalkyl, —C₁₋₄alkyl-(monocyclic cycloalkyl), monocyclic heterocyclyl, monocyclic heterocycloalkyl, monocyclic heteroaryl, or —C₁₋₄alkyl-(monocyclic heteroaryl), wherein each alkyl, phenyl, cycloalkyl, heterocyclyl, heterocycloalkyl or heteroaryl is optionally substituted with one, two, or three R^{d} substituents;
wherein each R^{d} substituent is independently C₁₋₄alkyl, C₁₋₄alkenyl, C₁₋₄alkynyl, —O—C₁₋₄alkyl, halo, cyano, nitro, azido, C₁₋₄haloalkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, ═NOR^{g}, —NR^{g}S(═O)₁₋₂R^{h}, —NR^{g}S(═O)₁₋₂NR^{g}R^{h}, ═NSO₂R^{g}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}—, —SR^{g}, —S(═O)R^{g}, —S(═O)₂R^{g}, —OS(═O)₁₋₂R^{g}, —S(═O)₁₋₂OR^{g}, or —S(═O)₁₋₂NR^{g}R^{h};
wherein R^{g} and R^{h} are each independently H or C₁₋₄alkyl;
each of R² and R³ is independently chosen from H, C₁₋₄alkyl, cycloalkyl, C₁₋₄alkylcycloalkyl, heterocyclyl, heterocycloalkyl, and heteroaryl optionally substituted with one, two, or three R^{j} substituents; or R² and R³ taken together with the nitrogen to which they are attached form a heterocyclyl, optionally substituted with one, two, three, or four R^{j} substituents, or further wherein any of the hydrogens bonded to carbon atoms are optionally replaced by deuterium;
wherein each R^{j} substituent is independently C₁₋₄alkyl, —OH, oxo, —NR^{k}R^{l}, halo, C₁₋₄haloalkyl, —O—C₁₋₄alkyl, or —O—C₁₋₄-haloalkyl;
where R^{k} and R^{l} are each independently H or C₁₋₄alkyl;
R⁴ is H, halo, —C(O)OH, C₁₋₄alkylNR^{x}R^{y}, or —C(O)NR^{x}R^{y}, or is a cycloalkyl, heterocyclyl, heterocycloalkyl, phenyl or heteroaryl, wherein each cycloalkyl, heterocyclyl, heterocycloalkyl, phenyl or heteroaryl is optionally substituted with one, two, or three R^{z} substituents;
wherein R^{x} is H or C₁₋₄alkyl and R^{y} is H, C₁₋₄alkyl, —O—C₁₋₄alkyl, —SO₂—R^{r}, C₁₋₄alkyl-SO₂—R^{r} monocyclic cycloalkyl, —C₁₋₄alkyl(monocyclic cycloalkyl), monocyclic heterocyclyl, or monocyclic heterocycloalkyl, each optionally substituted with one, two, or three R^{o} substituents;
or R^{x} and R^{y} taken together with the nitrogen to which they are attached form a monocyclic heterocyclyl, optionally substituted with C₁₋₄alkyl or —OC₁₋₄alkyl; and
each R^{z} substituent is independently C₁₋₄alkyl, halo, —NR^{p}R^{q}, —C(O)NR^{P}R^{q}, —OH, or —OC₁₋₄alkyl, wherein each alkyl is optionally substituted with —NR^{m}Rⁿ;
wherein R^{m} and Rⁿ are each independently H, C₁₋₄alkyl, C(O)C₁₋₂alkyl, C(O)C₁₋₂haloalkyl, C(O)C₁₋₂alkenyl, or R^{m} and Rⁿ taken together with the nitrogen to which they are attached form a monocyclic heterocycloalkyl, optionally substituted with one or two R^{o} substituents;
wherein each R^{o} substituent is independently C₁₋₄alkyl, —OH, —OC₁₋₄alkyl, halo, cyano, methylsulfonyl, —NR^{p}R^{q}, or —C(O)NR^{p}R^{q};
wherein R^{p} and R^{q} are each independently H, C₁₋₄alkyl, C₁₋₄alkylNH₂, C₁₋₄alkylNH(C₁₋₄alkyl), or C₁₋₄alkylN(C₁₋₄alkyl)₂;
wherein each R^{r} is independently C₁₋₄alkyl or NR^{p}R^{q}; and
R⁵ is H, C₁₋₄alkyl, halo, —OH, or —OC₁₋₄alkyl;
or a pharmaceutically acceptable salt or prodrug or prodrug thereof.

2. The compound of claim 1, wherein R^{1a} and R^{1b} are taken together with the nitrogen to which they are attached to form

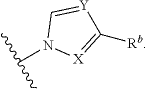

3. The compound of claim 1, wherein R^{1a} and R^{1b} are taken together with the nitrogen to which they are attached to form

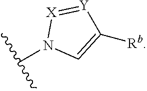

4. The compound of claim 1, wherein X is N and Y is CR^{a}.

5. The compound of claim 1, wherein X is CR^{a} and Y is N.

6. The compound of claim 1, wherein X is N and Y is N.

7. The compound of claim 1, wherein R^{a} is H, methyl, ethyl, propyl, isopropyl, butyl, isobutyl, sec-butyl, or tert-butyl.

8. The compound of claim 1, wherein R^{a} is H or methyl.

9. The compound of claim 1, wherein R^{a} is H.

10. The compound of claim 1, wherein R^{b} is optionally substituted phenyl.

11. The compound of claim 1, wherein R^{b} is tolyl.

12. The compound of claim 1, wherein R^{b} is phenyl.

13. The compound of claim 1, wherein R^{b} is optionally substituted pyridinyl or pyrimidinyl.

14. The compound of claim 1, wherein R^{b} is optionally substituted pyridinyl.

15. The compound of claim 1, wherein R^{b} is substituted with one or two R^{d} substituents.

16. The compound of claim 1, wherein R^{b} is methylpyridinyl, phenyl, m-tolyl, chlorophenyl, bromophenyl, methoxyphenyl.

17. The compound of claim 1, wherein R^{1a} is H or C₁₋₄alkyl; and R^{1b} is a 5-membered N-containing heteroaryl optionally substituted with R^{c}.

18. The compound of claim 1, wherein R^{1a} is H.

19. The compound of claim 1, wherein R^{1a} is C₁₋₄alkyl.

20. The compound of claim 1, wherein R^{1a} is methyl.

21. The compound of claim 1, wherein R^{1b} is pyrrolyl, pyrazolyl, imidazolyl, triazolyl, oxazolyl, isoxazolyl, thiazolyl, isothiazolyl, oxadiazolyl, thiadiazolyl, pyrazolopyridinyl, or indazolyl, each optionally substituted with R^{c}.

22. The compound of claim 1, wherein R^{1b} is pyrazolyl, imidazolyl, oxazolyl, oxadiazolyl or isoxazolyl, each optionally substituted with R^{c}.

23. The compound of claim 1, wherein R^{1b} is pyrazolyl, optionally substituted with R^{c}.

24. The compound of claim 1, wherein R^{1b} is

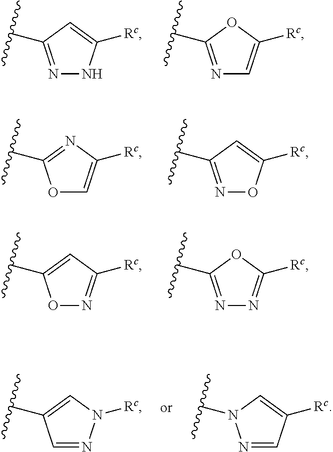

25. The compound of claim 1, wherein R^{1b} is

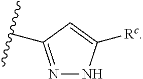

26. The compound of claim 1, wherein R^{c} is optionally substituted C₁₋₄alkyl.

27. The compound of claim 1, wherein R^{c} is methyl, ethyl, isopropyl, or trifluoromethyl.

28. The compound of claim 1, wherein R^{c} is optionally substituted phenyl.

29. The compound of claim 1, wherein R^{c} is phenyl or o-, m-, p-tolyl, fluorophenyl, methoxyphenyl, or trifluoromethoxyphenyl.

30. The compound of claim 1, wherein R^{c} is phenyl.

31. The compound of claim 1, wherein R^{c} is optionally substituted monocyclic cycloalkyl.

32. The compound of claim 1, wherein R^{c} is optionally substituted cyclopropyl, cyclobutyl, cyclopentyl, or cyclohexyl.

33. The compound of claim 1, wherein R^{c} is optionally substituted cyclopropyl.

34. The compound of claim 1, wherein R^{c} is optionally substituted monocyclic heterocycloalkyl.

35. The compound of claim 1, wherein R^{c} is optionally substituted cyclopropylmethyl, cyclobutylmethyl, cyclopentylmethyl, or cyclohexylmethyl.

36. The compound of claim 1, wherein R^{c} is optionally substituted monocyclic heterocyclyl.

37. The compound of claim 1, wherein R^{c} is optionally substituted pyrrolidinyl, tetrahydrofuranyl, piperidinyl, morpholinyl, or piperazinyl.

38. The compound of claim 1, wherein R^{c} is optionally substituted monocyclic heteroaryl.

39. The compound of claim 1, wherein R^{c} is optionally substituted pyrrolyl, imidazolyl, pyrazolyl, triazolyl, tetrazolyl, furanyl, thiophenyl, oxazolyl, isoxazolyl, thiazolyl, isothiazolyl, pyridinyl, pyrimidinyl, pyrazinyl, or pyridazinyl.

40. The compound of claim 1, wherein R^{c} is optionally substituted pyrazole, thiophenyl, imidazolyl, pyridinyl, or pyrimidinyl.

41. The compound of claim 1, wherein R^{c} is optionally substituted pyrazolyl.

42. The compound of claim 1, wherein R^{c} is optionally substituted pyridinyl.

43. The compound of claim 1, wherein R^{c} is methylpyridinyl.

44. The compound of claim 1, wherein R^{c} is optionally substituted —C₁₋₄alkyl-phenyl, —C₁₋₄alkyl-(monocyclic cycloalkyl), monocyclic heterocycloalkyl, or —C₁₋₄alkyl-(monocyclic heteroaryl).

45. The compound of claim 1, wherein R^{c} is optionally substituted with one or two R^{d} substituents and each R^{d} substituent is independently C₁₋₄alkyl, C₁₋₄alkenyl, C₁₋₄alkynyl, —O—C₁₋₄alkyl, halo, cyano, nitro, azido, C₁₋₄haloalkyl, —O—C₁₋₄-haloalkyl, —NR^{g}R^{h}, —NR^{g}C(═O)R^{h}, —NR^{g}C(═O)NR^{g}R^{h}, —NR^{g}C(═O)OR^{h}, ═NOR^{g}, —NR^{g}S(═O)₁₋₂R^{h}, —NR^{g}S(═O)₁₋₂NR^{g}R^{h}, ═NSO₂R^{g}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, or —S(═O)₁₋₂NR^{g}R^{h}.

46. The compound of claim 1, wherein each R^{d} substituent is independently C₁₋₄alkyl, —O—C₁₋₄alkyl, C₁₋₄haloalkyl, or halo.

47. The compound of claim 1, wherein each R^{d} substituent is independently methyl, ethyl, isopropyl, —CF₃, —OCH₃, —OCF₃, or fluoro.

48. The compound of claim 1, wherein R^{g} and R^{h} are each independently H or methyl.

49. The compound of claim 1, wherein each of R² and R³ are independently selected from H, pyrrolidinyl, piperidinyl, piperazinyl, and imidazolyl, wherein each pyrrolidinyl, piperidinyl, piperazinyl, and imidazolyl is optionally substituted with one R^{j} substituent.

50. The compound of claim 1, wherein R² and R³ taken together with the nitrogen to which they are attached form pyrrolidinyl, piperidinyl, piperazinyl, imidazolyl, morpholino, or thiomorpholino, each optionally substituted with one, two, three, or four R^{j} substituents.

51. The compound of claim 1, wherein R² and R³ taken together with the nitrogen to which they are attached form morpholino, imidazolyl, or piperazinyl, optionally substituted with one, two, three, or four R^{j} substituents.

52. The compound of claim 1, wherein R² and R³ taken together with the nitrogen to which they are attached form 2,2,6,6-tetrafluoro-morpholino, morpholino-3-one, morpholino-3-one, piperazinyl-2-one, piperazinyl-3-one, thiomorpholino-1,1-dioxide.

53. The compound of claim 1, wherein each R^{j} substituent is independently methyl, oxo, hydroxy, NH₂, —OCH₃, halo, —CF₃, or —OCF₃.

54. The compound of claim 1, wherein R² and R³ taken together with the nitrogen to which they are attached form morpholino in which 1 to 8 hydrogens are replaced with deuterium.

55. The compound of claim 1, wherein R^{k} and R^{l} are each independently H or methyl.

56. The compound of claim 1, wherein R⁴ is H.

57. The compound of claim 1, wherein R⁴ is chloro.

58. The compound of claim 1, wherein R⁴ is optionally substituted phenyl.

59. The compound of claim 1, wherein R⁴ is optionally substituted heteroaryl.

60. The compound of claim 1, wherein R⁴ is optionally substituted monocyclic heteroaryl.

61. The compound of claim 1, wherein R⁴ is optionally substituted pyrrolyl, imidazolyl, pyrazolyl, triazolyl, tetrazolyl, furanyl, oxazolyl, isoxazolyl, thiazolyl, isothiazolyl, pyridinyl, pyrimidinyl, pyrazinyl, or pyridazinyl.

62. The compound of claim 1, wherein R⁴ is 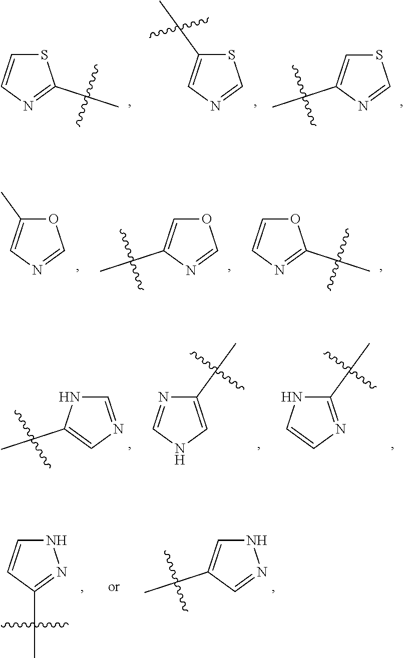
each optionally substituted with 1 or 2 R^{z} groups.

63. The compound of claim 1, wherein R⁴ is optionally substituted pyridinyl or pyrimidinyl.

64. The compound of claim 1, wherein R⁴ is optionally substituted pyridinyl.

65. The compound of claim 1, wherein R⁴ is pyridinyl.

66. The compound of claim 1, wherein R⁴ is optionally substituted pyrazolyl.

67. The compound of claim 1, wherein R⁴ is optionally substituted with one or two R^{z} substituents.

68. The compound of claim 1, wherein R⁴ is pyrazolyl optionally substituted with one or two R^{z} substituents.

69. The compound of claim 1, wherein R⁴ is phenyl or pyridyl, each optionally substituted with one or two substituents selected from C₁₋₄alkyl, —CF₃, fluoro, chloro, —OCH₃, and —OCF₃.

70. The compound of claim 1, wherein R⁴ is heterocyclyl, optionally substituted with one or two R^{z} substituents.

71. The compound of claim 1, wherein R⁴ is pyrrolidinyl, piperidinyl, piperazinyl, morpholino, or thiomorpholino, optionally substituted with one or two R^{z} substituents.

72. The compound of claim 1, wherein R⁴ is heterocycloalkyl, optionally substituted with one or two R^{z} substituents.

73. The compound of claim 1, wherein R⁴ is pyrrolidinylmethyl, piperidinylmethyl, piperazinylmethyl, morpholinomethyl, or thiomorpholinomethyl, optionally substituted with one or two R^{z} substituents.

74. The compound of claim 1, wherein R⁴ is 3-methyl-1H-pyrazol-5-yl, 3-methylisothiazol-5-yl, 2-methyl-1H-imidazol-5-yl, 1-methyl-pyrazol-4-yl, 1-methylpyrazol-3-yl, 1-((1-acetamido)-eth-2-yl)-5-methyl-pyrazol-3-yl, 1-((1-chloromethylamido)-eth-2-yl)-5-methyl-pyrazol-3-yl, 1-((1-acrylamido)-eth-2-yl)-5-methyl-pyrazol-3-yl, thiazol-2-yl, pyrazol-4-yl, pyrazol-1-yl, oxazol-2-yl, 3-(1-N,N-dimethyl-eth-2-yl)-4-methyl-pyrazol-1-yl, or pyridinyl.

75. The compound of claim 1, wherein R⁴ is C₁₋₄alkylNR^{x}R^{y}.

76. The compound of claim 1, wherein R⁴ is CH₂NR^{x}R^{y}.

77. The compound of claim 1, wherein R⁴ is —C(O)NR^{x}R^{y}.

78. The compound of claim 1, wherein R^{x} is H.

79. The compound of claim 1, wherein R^{x} is methyl or ethyl, optionally substituted with one, two, or three R^{o} substituents.

80. The compound of claim 1, wherein R^{x} is methyl.

81. The compound of claim 1, wherein R^{y} is H.

82. The compound of claim 1, wherein R^{y} is C₁₋₄alkyl, optionally substituted with one, two, or three R^{o} substituents.

83. The compound of claim 1, wherein R^{y} is methyl, ethyl, propyl, or isopropyl, each optionally substituted with one, two, or three R^{o} substituents.

84. The compound of claim 1, wherein R^{y} is H, methyl, ethyl, methyoxy, or methoxyethyl.

85. The compound of claim 1, wherein R^{y} is methyl.

86. The compound of claim 1, wherein R^{y} is —SO₂—R^{r} or C₁₋₄alkyl-SO₂—R^{r}.

87. The compound of claim 1, wherein R^{y} is —SO₂—R^{r}, C₁₋₄alkyl-SO₂—R^{r}; and R^{r} is CH₃ or NH₂, NHCH₃, or N(CH₃)₂.

88. The compound of claim 1, wherein R^{y} is —SO₂-methyl, C₂₋₄alkyl-SO₂—N(CH₃)₂.

89. The compound of claim 1, wherein R^{y} is monocyclic cycloalkyl or —C₁₋₂alkyl(monocyclic cycloalkyl), each optionally substituted with one, two, or three R^{o} substituents.

90. The compound of claim 1, wherein R^{y} is monocyclic cycloalkyl, optionally substituted with one, two, or three R^{o} substituents.

91. The compound of claim 1, wherein R^{y} is cyclopropyl, cyclobutyl, cyclopentyl, or cyclohexyl, each optionally substituted with one, two, or three R^{o} substituents.

92. The compound of claim 1, wherein R^{y} is cyclopropyl.

93. The compound of claim 1, wherein R^{y} is cyclopropyl, cyclobutyl, cyclopentyl, cyclopropylmethyl, 1-cyclopropylethyl, 2-cyclopropylethyl, cyclobutylmethyl, or cyclopentylmethyl.

94. The compound of claim 1, wherein R^{y} is monocyclic heterocyclyl, optionally substituted with one, two, or three R^{o} substituents.

95. The compound of claim 1, wherein R^{y} is optionally substituted azetidinyl, oxetanyl, pyrrolidinyl, piperidinyl, morpholinyl, piperazinyl, azepanyl, azocanyl, tetrahydrofuranyl, or tetrahydropyranyl, optionally substituted with methyl.

96. The compound of claim 1, wherein R^{y} is monocyclic heterocycloalkyl, optionally substituted with one, two, or three R^{o} substituents.

97. The compound of claim 1, wherein R^{y} is optionally substituted azetidinylmethyl, oxetanylmethyl, pyrrolidinylmethyl, piperidinylmethyl, morpholinylmethyl, or piperazinylmethyl, optionally substituted with methyl.

98. The compound of claim 1, wherein one of R^{x} and R^{y} is H and the other is —CH₃.

99. The compound of claim 1, wherein both of R^{x} and R^{y} is H.

100. The compound of claim 1, wherein both of R^{x} and R^{y} is —CH₃.

101. The compound of claim 1, wherein R^{x} and R^{y} taken together with the nitrogen to which they are attached form a monocyclic heterocyclyl, optionally substituted with C₁₋₄alkyl.

102. The compound of claim 1, wherein R^{x} and R^{y} are taken together with the nitrogen to which they are attached to form azetidinyl, pyrrolidinyl, piperidinyl, piperazinyl, morpholinyl, or thiomorpholinyl, each optionally substituted with methyl.

103. The compound of claim 1, wherein each R^{z} is independently C₁₋₄alkyl, halo, —OH, or —OC₁₋₄alkyl, wherein each alkyl is optionally substituted with —NR^{m}Rⁿ.

104. The compound of claim 1, wherein each R^{z} is independently —CH₃, —OH, halo, or —OCH₃.

105. The compound of claim 1, wherein R^{z} is C₂₋₄alkyl substituted with —NR^{m}Rⁿ or OCH₃.

106. The compound of claim 1, wherein each R^{z} substituent is independently —NR^{p}R^{q}, —C(O)NR^{p}R^{q}.

107. The compound of claim 1, wherein each R^{z} substituent is methyl, ethyl, isopropyl, —CF₃, fluoro, chloro, —OCH₃, —OCF₃, methylamino, ethylamino, propylamino, butylamino, aminomethyl, aminoethyl, aminopropyl, aminobutyl, dimethylamino, dimethylaminomethyl, dimethylaminoethyl, dimethylaminopropyl, dimethylaminobutyl, —C(O)methylamino, —C(O)ethylamino, —C(O)propylamino, —C(O)butylamino, —C(O)dimethylamino, —C(O)dimethylaminomethyl, —C(O)dimethylaminoethyl, —C(O)dimethylaminopropyl, or —C(O)dimethylaminobutyl.

108. The compound of claim 1, wherein R^{m} and Rⁿ are each independently H, C₁₋₄alkyl, C(O)CH₃, C(O)CH₂Cl, or C(O)CH₂CH₂.

109. The compound of claim 1, wherein R^{m} and Rⁿ are each H.

110. The compound of claim 1, wherein R^{m} and Rⁿ are each methyl.

111. The compound of claim 1, wherein R^{m} and Rⁿ taken together with the nitrogen to which they are attached form a monocyclic heterocyclyl, optionally substituted with one or two R^{o} substituents.

112. The compound of claim 1, wherein R^{m} and Rⁿ taken together with the nitrogen to which they are attached form pyrrolidinyl, piperidinyl, piperazinyl, morpholino, thiomorpholino, or thiomorpholino-1,1-dioxide, each optionally substituted with one or two R^{o} substituents.

113. The compound of claim 1, wherein R^{m} and Rⁿ taken together with the nitrogen to which they are attached form pyrrolidinyl, piperidinyl, piperazinyl, or morpholino, each optionally substituted with methyl.

114. The compound of claim 1, wherein each R^{o} substituent is C₁₋₄alkyl, or —NR^{p}R^{q}.

115. The compound of claim 1, wherein R^{p} and R^{q} are each independently H, methyl, C₁₋₄alkylNH₂, C₁₋₄alkylNHCH₃, or C₁₋₄alkylN(CH₃)₂.

116. The compound of claim 1, wherein R^{p} and R^{q} are each independently H or methyl.

117. The compound of claim 1, wherein R⁵ is H, methyl, ethyl, chloro, bromo, fluoro, —OH, or —OCH₃.

118. The compound of claim 1, wherein R⁵ is H.

119. The compound of claim 1, wherein the compound of Formula (I) or the pharmaceutically acceptable salt thereof is a compound of Formula (II): 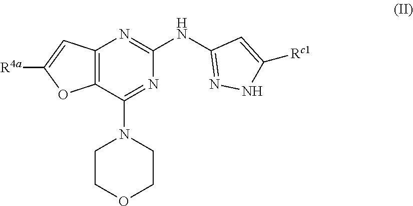
wherein
R^{c1} is phenyl or pyridyl, each optionally substituted with one or two substituents selected from C₁₋₄alkyl, —CF₃, fluoro, chloro, —OCH₃, and —OCF₃; and
R^{4a} is C₁₋₄alkylNR^{x}R^{y} or C(O)NR^{x}R^{y} wherein R^{x} and R^{y} are as defined herein; or is phenyl, pyrazolyl, or pyridyl, each optionally substituted with one or two R^{z} groups;
or a pharmaceutically acceptable salt or prodrug thereof.

120. The compound of claim 1, wherein the compound of Formula (I) or the pharmaceutically acceptable salt thereof is a compound of Formula (III): 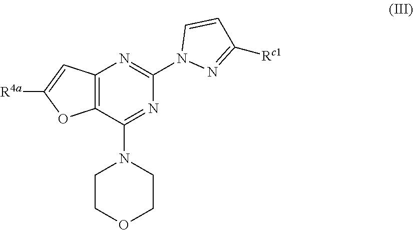
wherein
R^{c1} is phenyl or pyridyl, each optionally substituted with one or two substituents selected from C₁₋₄alkyl, —CF₃, fluoro, chloro, —OCH₃, and —OCF₃; and
R^{4a} is C₁₋₄alkylNR^{x}R^{y} or —C(O)NR^{x}R^{y} wherein R^{x} and R^{y} are as defined herein; or is phenyl, pyrazolyl, or pyridyl, each optionally substituted with one or two R^{z} groups;
or a pharmaceutically acceptable salt or prodrug thereof.

121. The compound of claim 1, wherein the compound of Formula (I) or the pharmaceutically acceptable salt thereof is a compound of Formula (IV): 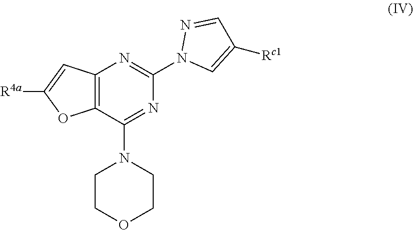
wherein
R^{c1} is phenyl or pyridyl, each optionally substituted with one or two substituents selected from C₁₋₄alkyl, —CF₃, fluoro, chloro, —OCH₃, and —OCF₃; and
R^{4a} is C₁₋₄alkylNR^{x}R^{y} or —C(O)NR^{x}R^{y} wherein R^{x} and R^{y} are as defined herein; or is phenyl, pyrazolyl, or pyridyl, each optionally substituted with one or two R^{z} groups;
or a pharmaceutically acceptable salt or prodrug thereof.

122. The compound of any one of claims 119 to 121, wherein R^{c1} is phenyl or pyridyl, each optionally substituted with methyl, —CF₃, Cl, Br, or OCH₃.

123. The compound of any one of claims 119-121, wherein R^{c1} is phenyl.

124. The compound of any one of claims 119-121, wherein R^{c1} is tolyl.

125. The compound of any one of claims 119-121, wherein R^{c1} is pyridyl optionally substituted with methyl or —CF₃.

126. The compound of any one of claims 119-121, wherein R^{4a} is pyridyl, optionally substituted with one or two R^{z} groups.

127. The compound of any one of claims 119-121, wherein R^{4a} is pyridyl.

128. The compound of any one of claims 119-121, wherein R^{4a} is pyrazolyl optionally substituted with one or two R^{z} groups.

129. The compound of any one of claims 119-121 and 129, wherein each R^{z} is independently methyl, ethyl, isopropyl, —CF₃, fluoro, chloro, —OCH₃, —OCF₃, methylamino, ethylamino, propylamino, butylamino, aminomethyl, aminoethyl, aminopropyl, aminobutyl, dimethylamino, dimethylaminomethyl, dimethylaminoethyl, dimethylaminopropyl, dimethylaminobutyl, —C(O)methylamino, —C(O)ethylamino, —C(O)propylamino, —C(O)butylamino, —C(O)dimethylamino, —C(O)dimethylaminomethyl, —C(O)dimethylaminoethyl, —C(O)dimethylaminopropyl, or —C(O)dimethylaminobutyl.

130. The compound of any one of claims 119-121, wherein R^{4a} is 3-methyl-1H-pyrazol-5-yl, 3-methylisothiazol-5-yl, 2-methyl-1H-imidazol-5-yl, 1-methyl-pyrazol-4-yl, 1-methylpyrazol-3-yl, 1-((1-acetamido)-eth-2-yl)-5-methyl-pyrazol-3-yl, 1-((1-chloromethylamido)-eth-2-yl)-5-methyl-pyrazol-3-yl, 1-((1-acrylamido)-eth-2-yl)-5-methyl-pyrazol-3-yl, thiazol-2-yl, pyrazol-4-yl, pyrazol-1-yl, oxazol-2-yl, or 3-(1-N,N-dimethyl-eth-2-yl)-4-methyl-pyrazol-1-yl.

131. The compound of any one of claims 119-121, wherein R^{4a} is —C(O)NR^{x}R^{y} wherein R^{x} is H or C₁₋₄alkyl and R^{y} is H, C₁₋₄alkyl, —O—C₁₋₄alkyl, —SO₂—R^{r}, C₁₋₄alkyl-SO₂—R^{r} monocyclic cycloalkyl, —C₁₋₄alkyl(monocyclic cycloalkyl), monocyclic heterocyclyl, or monocyclic heterocycloalkyl, each optionally substituted with one, two, or three R^{o} substituents; and Rand R^{o} are as defined herein.

132. The compound of any one of claims 119-121, wherein R^{4a} is —C(O)NR^{x}R^{y} wherein R^{x} is H or methyl; and R^{y} is H, methyl, ethyl, butyl, isopropyl, methoxy, —SO₂-methyl, C₂₋₄alkyl-SO₂-methyl, C₂₋₄alkyl-SO₂—N(CH₃)₂, cyclopropyl, cyclobutyl, cyclopentyl, cyclopropylmethyl, 1-cyclopropylethyl, 2-cyclopropylethyl, cyclobutylmethyl, cyclopentylmethyl, azetidinyl, oxetanyl, pyrrolidinyl, piperidinyl, morpholinyl, piperazinyl, azepanyl, azocanyl, tetrahydrofuranyl, tetrahydropyranyl, substituted azetidinylmethyl, oxetanylmethyl, pyrrolidinylmethyl, piperidinylmethyl, morpholinylmethyl, or piperazinylmethyl, each optionally substituted with one, two, or three methyl, methoxy, fluoro or amino groups.

133. A compound selected from Table 1, and/or pharmaceutically acceptable salts thereof.

134. A compound of any one of the preceding claims, wherein one or more hydrogen atoms attached to carbon atoms of the compound are replaced by deuterium atoms.

135. A pharmaceutical composition comprising a compound and/or a pharmaceutically acceptable salt or prodrug of any one of claims 1 to 134 and a pharmaceutically acceptable excipient.

136. A method of inhibiting PIKfyve and/or a PI3 kinase in a subject in need thereof comprising administering to the subject an effective amount of a compound of any one of claims 1 to 133, or a pharmaceutical composition of claim 135.

137. A method of treating a neurological disease associated with PIKfyve activity and/or PI3 kinase activity in a subject in need thereof comprising administering to the subject an effective amount of a compound of any one of claims 1 to 134, or a pharmaceutical composition of claim 134.

138. The method of claim 137, wherein the disease is associated with PIKfyve activity.

139. The method of claim 137, wherein the disease is amyotrophic lateral sclerosis (ALS), primary lateral sclerosis (PLS), Charcot-Marie-Tooth (CMT; including type 4J (CMT4J)), and Yunis-Varon syndrome, autophagy, polymicrogyria (including polymicrogyria with seizures), temporo-occipital polymicrogyria, Pick's disease, Parkinson's disease, Parkinson's disease with Lewy bodies, dementia with Lewy bodies, Lewy body disease, fronto-temporal dementia, diseases of neuronal nuclear inclusions of polyglutamine and intranuclear inclusion bodies, disease of Marinesco and Hirano bodies, tauopathy, Alzheimer's disease, neurodegeneration, spongiform neurodegeneration, peripheral neuropathy, leukoencephalopathy, inclusion body disease, progressive supranuclear palsy, corticobasal syndrome, chronic traumatic encephalopathy, traumatic brain injury (TBI), cerebral ischemia, Guillain-Barré Syndrome, chronic inflammatory demyelinating polyneuropathy, multiple sclerosis, a lysosomal storage disease, Fabry's disorder, Gaucher's disorder, Niemann Pick C disease, Tay-Sachs disease, and Mucolipidosis type IV, neuropathy, Huntington's disease, a psychiatric disorder, ADHD, schizophrenia, a mood disorder, major depressive disorder, depression, bipolar disorder I, or bipolar disorder II.

140. The method of claim 137, wherein the disease is ALS, FTD, Alzheimer's disease, Parkinson's disease, Huntington's disease, or CMT.

141. The method of claim 137, wherein the disease is ALS.

142. The method of claim 137, wherein the disease is a tauopathy such as Alzheimer's disease, progressive supranuclear palsy, corticobasal syndrome, frontotemporal dementia, or chronic traumatic encephalopathy.

143. The method of claim 137, wherein the disease is a lysosomal storage disease such as Fabry's disorder, Gaucher's disorder, Niemann Pick C disease, Tay-Sachs disease, or Mucolipidosis type IV.

144. The method of claim 137, wherein the disease is a psychiatric disorder such as ADHD, schizophrenia, or mood disorders such as major depressive disorder, depression, bipolar disorder I, or bipolar disorder II.

145. The method of claim 137, wherein the disease is associated with PI3K activity.

146. The method of claim 145, wherein the PI3K is PI3Kα, PI3Kβ, PI3Kδ, and/or PI3Kγ.

147. A compound of any one of claims 1 to 134 for use as a medicament.

148. The compound of claim 147, wherein the compound is for use in treating a neurological disease treatable by inhibition of PIKfyve and/or a PI3 kinase.

149. Use of a compound of any one of claims 1 to 134 in the manufacture of a medicament for treating a disease in a subject in which PIKfyve or PI3K contributes to the pathology and/or symptoms of the disease.
